SEQUENCE LISTING (185 sequences)

SEQ ID NO: 1 - protein, 914 aa, synthetic construct
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTHHHHHH

SEQ ID NO: 2 - protein, 1137 aa, synthetic construct
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGGGGSCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 914 aa, synthetic construct
ECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTHHHHHH

SEQ ID NO: 4 - protein, 121 aa, synthetic construct
QVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRNMGYSGWLDYWGQGTLVTVSS

SEQ ID NO: 5 - DNA, 363 bp, synthetic construct
  source 1..363: mol_type = other DNA
caggtgcagctggtggagtctgggggaggcgtggtccagcctgggaggtccctgagactctcctgtgcagcctctggattcaccttcagtagccatggcatgcactgggtccgccaggctccaggcaaggggctggagtgggtggcagttatatcatatgatgaaagtgataaatattatgcagactccgtgaagggccgattcaccatctccagagaccattccaagaacacgctgtatctgcaaatgaacagcctgagagctgaggacacggctgtatattactgtgtgaaagatcggaatatgggatatagtggctggcttgactactggggccagggaaccctggtcaccgtctcctca

SEQ ID NO: 6 - protein, 5 aa, synthetic construct
SHGMH

SEQ ID NO: 7 - protein, 17 aa, synthetic construct
VISYDESDKYYADSVKG

SEQ ID NO: 8 - protein, 12 aa, synthetic construct
DRNMGYSGWLDY

SEQ ID NO: 9 - protein, 121 aa, synthetic construct
QVQLVESGGGVVQPGKSLRLSCAASGFTFSSSGLHWVRQAPGKGLEWVAVISYDTSNKYYADSVKGRFTISRDNSKNTLYLQMNSLRAEDTAVYYCAKDLNRGYDWGFDYWGQGTLVTVSS

SEQ ID NO: 10 - DNA, 363 bp, synthetic construct
  source 1..363: mol_type = other DNA
caggtgcagctggtggagtctgggggaggcgtggtccagcctgggaagtccctgagactctcctgtgcagcctctggattcaccttcagtagttctggcttgcactgggtccgccaggctccaggcaagggactggagtgggtggcagttatatcatatgatacaagtaataaatactatgcagactccgtgaagggccgattcaccatctccagagacaattccaagaacacgctgtatctgcaaatgaacagcctgagagctgaggacacggctgtatattactgtgcgaaagatctaaatcgtggctatgattgggggtttgactactggggccagggaaccctggtcaccgtctcctca

SEQ ID NO: 11 - protein, 5 aa, synthetic construct
SSGLH

SEQ ID NO: 12 - protein, 17 aa, synthetic construct
VISYDTSNKYYADSVKG

SEQ ID NO: 13 - protein, 12 aa, synthetic construct
DLNRGYDWGFDY

SEQ ID NO: 14 - protein, 120 aa, synthetic construct
QVQLVESGGGVVQPGRSLRLSCAASGFTFSNYGMHWVRQAPGKGLEWVAVISYDGVDKYYADSVKGRFTISRDNSKNTLYLQMNSLRAEDTAMYYCAKDRISYNWNLDYWGQGTLVTVSS

SEQ ID NO: 15 - DNA, 360 bp, synthetic construct
  source 1..360: mol_type = other DNA
caggtgcagctggtggagtctgggggaggcgtggtccagcctgggaggtccctgagactctcctgtgcagcctctggattcaccttcagtaactatggcatgcactgggtccgccaggctccaggcaaggggctggagtgggtggcagttatatcatatgatggcgttgataagtactatgcagactccgtgaagggccgattcaccatctccagagacaattccaagaacacgctgtatctgcaaatgaacagcctgagagctgaggacacggctatgtattactgtgcgaaagatcggatttcctataactggaaccttgactactggggccagggaaccctggtcaccgtctcctca

SEQ ID NO: 16 - protein, 5 aa, synthetic construct
NYGMH

SEQ ID NO: 17 - protein, 17 aa, synthetic construct
VISYDGVDKYYADSVKG

SEQ ID NO: 18 - protein, 11 aa, synthetic construct
DRISYNWNLDY

SEQ ID NO: 19 - protein, 121 aa, synthetic construct
QVQLVESGGGVVQPGRSLRLSCVASGFTFSNYGLHWVRQAPGKGLEWVAAISYDGSDKYYADPVKGRFTISRDTTKNTLSLQMNSLRAEDTAVYYCAKERYRGYDWDFDYWGQGTLVTVSS

SEQ ID NO: 20 - DNA, 363 bp, synthetic construct
  source 1..363: mol_type = other DNA
caggtgcagttggtggagtctgggggaggcgtggtccagcctgggaggtccctgagactctcctgtgtagcctctggattcaccttcagtaactatggcctacactgggtccgccaggctccaggcaaggggctggagtgggtggcagctatatcatatgatggaagtgataaatactatgcagaccccgtgaagggccgattcaccatctccagagacacgaccaagaacacgctgtctctgcaaatgaacagtctgagagctgaggacacggctgtgtattactgtgcgaaagaaagatatcgtggctacgattgggactttgactactggggccagggaaccctggtcaccgtctcctca

SEQ ID NO: 21 - protein, 5 aa, synthetic construct
NYGLH

SEQ ID NO: 22 - protein, 17 aa, synthetic construct
AISYDGSDKYYADPVKG

SEQ ID NO: 23 - protein, 12 aa, synthetic construct
ERYRGYDWDFDY

SEQ ID NO: 24 - protein, 121 aa, synthetic construct
QVQLVESGGGVVQPGRSLRLSCAASGFTFSSYGLHWVRQAPGKGLEWVAVISFDGSNKYYADSVKGRFTISRDTSKNTLSLQMNSLRAEDTAVYYCAKERSRGYDWDFDYWGQGILVTVSS

SEQ ID NO: 25 - DNA, 363 bp, synthetic construct
  source 1..363: mol_type = other DNA
caggtgcagctggtggagtctgggggaggcgtggtccagcctgggaggtccctgagactctcctgtgcagcctctggattcaccttcagtagctatggcctacactgggtccgccaggctccaggcaaggggctggagtgggtggcagttatatcatttgatggaagtaataaatactatgcagactccgtgaagggccgattcaccatctccagagacacttccaagaacacgctgtctctgcaaatgaacagcctgagagctgaggacacggctgtgtattactgtgcgaaagaaagatctcgtggctacgattgggactttgactactggggccagggaatcctggtcaccgtctcctca

SEQ ID NO: 26 - protein, 5 aa, synthetic construct
SYGLH

SEQ ID NO: 27 - protein, 17 aa, synthetic construct
VISFDGSNKYYADSVKG

SEQ ID NO: 28 - protein, 12 aa, synthetic construct
ERSRGYDWDFDY

SEQ ID NO: 29 - protein, 121 aa, synthetic construct
QVRLVESGGGVVQPGRSLRLSCAASGFTFSNYGMHWVRQAPGKGLEWVAVISYDGSDKYYPDSVKGRFTISRDNSRNTLYLQMNSLSTEDTAVYYCAKDQYRGYDGTFDYWGQGTLVTVSS

SEQ ID NO: 30 - DNA, 363 bp, synthetic construct
  source 1..363: mol_type = other DNA
caggtgcggctggtggagtcggggggaggcgtggtccagcctgggaggtccctgagactctcctgtgcagcctctggattcaccttcagtaactatggcatgcactgggtccgccaggctccaggcaaggggctggagtgggtggcagttatatcatatgatggaagtgataagtactatccagactccgtgaagggccgcttcaccatctccagagacaattccagaaacacgctgtatctgcaaatgaacagcctgagtactgaggacacggctgtttattactgtgcgaaagatcaatatcgtggctacgatgggacttttgactactggggccagggaaccctggtcaccgtctcctca

SEQ ID NO: 31 - protein, 5 aa, synthetic construct
NYGMH

SEQ ID NO: 32 - protein, 17 aa, synthetic construct
VISYDGSDKYYPDSVKG

SEQ ID NO: 33 - protein, 12 aa, synthetic construct
DQYRGYDGTFDY

SEQ ID NO: 34 - protein, 120 aa, synthetic construct
QVQLVESGGGVVQPGRSLRLSCAASGFIFSSYNMHWVRQAPGKGLEWVAVISYDRVNKYYADSVKGRFTISRDNSKNTLYLQMNSLRVEDTAVYYCAKERERYFDWLDYWGQGTLVTVSS

SEQ ID NO: 35 - DNA, 360 bp, synthetic construct
  source 1..360: mol_type = other DNA
caggtgcagctggtggagtctgggggaggcgtggtccagcctgggaggtccctgagactctcctgtgcagcctctggattcatcttcagtagctataacatgcactgggtccgccaggctccaggcaaggggctggagtgggtggcagttatatcatatgatagagttaataaatactatgcagactccgtgaagggccgattcaccatctccagagacaattccaagaacacgctgtatctgcaaatgaacagcctgagagttgaggacacggctgtgtattactgtgcgaaagaaagggaacgatattttgactggttggactactggggccagggaaccctggtcaccgtctcctca

SEQ ID NO: 36 - protein, 5 aa, synthetic construct
SYNMH

SEQ ID NO: 37 - protein, 17 aa, synthetic construct
VISYDRVNKYYADSVKG

SEQ ID NO: 38 - protein, 11 aa, synthetic construct
ERERYFDWLDY

SEQ ID NO: 39 - protein, 121 aa, synthetic construct
QVQLVESGGGVVQPGRSLRLSCAASGFTFSSYGMHWVRQAPGKGLEWVAVISNDGSYKYYVDSVTGRFTISRDNSKNTLYLQMNSLRAEDTAVYYCAKDRNMGYSGWFDYWGQGSLVTVSS

SEQ ID NO: 40 - DNA, 363 bp, synthetic construct
  source 1..363: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaagttataaatactatgtagactccgtgacgggccgattcaccatctccagagacaattccaagaacacgctgtatctgcaaatgaacagcctgagagctgaggacacggctgtgtattactgtgcgaaagatcggaatatgggatatagtggctggtttgactactggggccagggatccctggtcaccgtctcctca

SEQ ID NO: 41 - protein, 5 aa, synthetic construct
SYGMH

SEQ ID NO: 42 - protein, 17 aa, synthetic construct
VISNDGSYKYYVDSVTG

SEQ ID NO: 43 - protein, 12 aa, synthetic construct
DRNMGYSGWFDY

SEQ ID NO: 44 - protein, 121 aa, synthetic construct
QVQLVESGGGVVQPGRSLRLSCAASGFTFSSHGMHWVRQAPGKGLEWVAVISNDGSYKYYADSVTGRFTISRDNSKNTLYLQMNSLRAEDTAVYYCAKDRNMGYSGWFDYWGQGSLVTVSS

SEQ ID NO: 45 - DNA, 363 bp, synthetic construct
  source 1..363: mol_type = other DNA
caggtgcagctggtggagtctgggggaggcgtggtccagcctgggaggtccctgagactctcctgtgcagcctctggattcaccttcagtagccatggcatgcactgggtccgccaggctccaggcaaggggctggagtgggtggcagttatatcaaatgatggaagttataaatactatgcagactccgtgacgggccgattcaccatctccagagacaattccaagaacacgctgtatctgcaaatgaacagcctgagagctgaggacacggctgtgtattactgtgcgaaagatcggaatatgggatacagtggctggtttgactactggggccagggatccctggtcaccgtctcctca

SEQ ID NO: 46 - protein, 5 aa, synthetic construct
SHGMH

SEQ ID NO: 47 - protein, 17 aa, synthetic construct
VISNDGSYKYYADSVTG

SEQ ID NO: 48 - protein, 12 aa, synthetic construct
DRNMGYSGWFDY

SEQ ID NO: 49 - protein, 122 aa, synthetic construct
QVQLVESGGGVVQPGRSLRLSCAASGFTFSNYGMHWVRQAPGKGLEWVAVISNDGSEKYYADSVKGRFTISRDSSKNTLYLQMNSLRAEDTAVYYCAKAPWGSGTTGRLDYWGQGTLVTVSS

SEQ ID NO: 50 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtaactatggcatgcactgggtccgccaggctccaggcaaggggctggagtgggtggctgttatatcaaatgatggaagtgaaaaatactatgcagactccgtgaagggccgattcaccatctccagagacagttccaagaacacgctctatctacagatgaacagcctgagagctgaggacacggctgtgtattactgtgcgaaagccccttggggaagtggaacgacggggaggcttgactactggggccagggaaccctggtcaccgtctcctca

SEQ ID NO: 51 - protein, 5 aa, synthetic construct
NYGMH

SEQ ID NO: 52 - protein, 17 aa, synthetic construct
VISNDGSEKYYADSVKG

SEQ ID NO: 53 - protein, 13 aa, synthetic construct
APWGSGTTGRLDY

SEQ ID NO: 54 - protein, 121 aa, synthetic construct
QFQLQESGPGLVKPSETLSLTCTVSGGSISTSSYYWGWIRQPPGKGLEWIGTIYYSGTTYYNPSLKSRVTISVDTSKNQFSLKLKSVTAADTSIYYCARHWPKYNSWFDPWGQGILVTVSS

SEQ ID NO: 55 - DNA, 363 bp, synthetic construct
  source 1..363: mol_type = other DNA
caatttcagctacaggagtcgggcccaggactggtgaagccttcggagaccctgtccctcacctgcactgtctctggtggctccatcagcactagtagttactactggggctggatccgccagcccccagggaaggggctggagtggattgggactatctattatagtgggaccacctactacaacccgtccctcaagagtcgagtcaccatatccgtagacacgtccaagaaccagttctccctgaagctgaagtctgtgaccgccgcagacacgtctatctattactgtgcgagacattggcccaagtacaactcctggttcgacccctggggccagggaattctggtcaccgtctcctca

SEQ ID NO: 56 - protein, 7 aa, synthetic construct
TSSYYWG

SEQ ID NO: 57 - protein, 16 aa, synthetic construct
TIYYSGTTYYNPSLKS

SEQ ID NO: 58 - protein, 11 aa, synthetic construct
HWPKYNSWFDP

SEQ ID NO: 59 - protein, 121 aa, synthetic construct
QFQLQESGPGLVKPSETLSLTCTVSGGSISTSSYYWGWIRQPPGKGLEWIGTIYFSGTTYYNPSLTSRVSMSVDTSKNQFSLKLNSVTAADTSIYYCARHWPKYNSWFDPWGQGFLVTVSS

SEQ ID NO: 60 - DNA, 363 bp, synthetic construct
  source 1..363: mol_type = other DNA
caatttcaactacaggagtcgggcccaggactggtgaagccttcggagaccctgtccctcacctgcactgtctctggtggctccatcagcactagtagttactactggggctggatccgccagcccccagggaaggggctggagtggattgggactatctattttagtgggaccacctactacaacccgtccctcacgagtcgagtcagcatgtccgtagacacgtccaagaaccagttctccctgaagctgaactctgtgaccgccgcagacacgtctatctattactgtgcgagacattggcccaagtacaactcctggttcgacccctggggccagggatttctggtcaccgtctcctca

SEQ ID NO: 61 - protein, 7 aa, synthetic construct
TSSYYWG

SEQ ID NO: 62 - protein, 16 aa, synthetic construct
TIYFSGTTYYNPSLTS

SEQ ID NO: 63 - protein, 11 aa, synthetic construct
HWPKYNSWFDP

SEQ ID NO: 64 - protein, 107 aa, synthetic construct [sequence omitted]

SEQ ID NO: 65 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagtagttacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaa

SEQ ID NO: 66 - protein, 11 aa, synthetic construct
RASQSVSSYLA

SEQ ID NO: 67 - protein, 7 aa, synthetic construct
DASNRAT

SEQ ID NO: 68 - protein, 9 aa, synthetic construct
QQRSNWPPT

SEQ ID NO: 69 - protein, 1390 aa, synthetic construct
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 70 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 107 aa, synthetic construct [sequence omitted]

SEQ ID NO: 72 - protein, 121 aa, synthetic construct
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRNMGYSGWLDYWGQGTLVTVSS

SEQ ID NO: 73 - protein, 121 aa, synthetic construct
EVQLVESGGGVVQPGKSLRLSCAASGFTFSSSGLHWVRQAPGKGLEWVAVISYDTSNKYYADSVKGRFTISRDNSKNTLYLQMNSLRAEDTAVYYCAKDLNRGYDWGFDYWGQGTLVTVSS

SEQ ID NO: 74 - protein, 120 aa, synthetic construct
EVQLVESGGGVVQPGRSLRLSCAASGFTFSNYGMHWVRQAPGKGLEWVAVISYDGVDKYYADSVKGRFTISRDNSKNTLYLQMNSLRAEDTAMYYCAKDRISYNWNLDYWGQGTLVTVSS

SEQ ID NO: 75 - protein, 121 aa, synthetic construct
EVQLVESGGGVVQPGRSLRLSCVASGFTFSNYGLHWVRQAPGKGLEWVAAISYDGSDKYYADPVKGRFTISRDTTKNTLSLQMNSLRAEDTAVYYCAKERYRGYDWDFDYWGQGTLVTVSS

SEQ ID NO: 76 - protein, 121 aa, synthetic construct
EVQLVESGGGVVQPGRSLRLSCAASGFTFSSYGLHWVRQAPGKGLEWVAVISFDGSNKYYADSVKGRFTISRDTSKNTLSLQMNSLRAEDTAVYYCAKERSRGYDWDFDYWGQGILVTVSS

SEQ ID NO: 77 - protein, 121 aa, synthetic construct
EVRLVESGGGVVQPGRSLRLSCAASGFTFSNYGMHWVRQAPGKGLEWVAVISYDGSDKYYPDSVKGRFTISRDNSRNTLYLQMNSLSTEDTAVYYCAKDQYRGYDGTFDYWGQGTLVTVSS

SEQ ID NO: 78 - protein, 120 aa, synthetic construct
EVQLVESGGGVVQPGRSLRLSCAASGFIFSSYNMHWVRQAPGKGLEWVAVISYDRVNKYYADSVKGRFTISRDNSKNTLYLQMNSLRVEDTAVYYCAKERERYFDWLDYWGQGTLVTVSS

SEQ ID NO: 79 - protein, 121 aa, synthetic construct
EVQLVESGGGVVQPGRSLRLSCAASGFTFSSYGMHWVRQAPGKGLEWVAVISNDGSYKYYVDSVTGRFTISRDNSKNTLYLQMNSLRAEDTAVYYCAKDRNMGYSGWFDYWGQGSLVTVSS

SEQ ID NO: 80 - protein, 121 aa, synthetic construct
EVQLVESGGGVVQPGRSLRLSCAASGFTFSSHGMHWVRQAPGKGLEWVAVISNDGSYKYYADSVTGRFTISRDNSKNTLYLQMNSLRAEDTAVYYCAKDRNMGYSGWFDYWGQGSLVTVSS

SEQ ID NO: 81 - protein, 122 aa, synthetic construct
EVQLVESGGGVVQPGRSLRLSCAASGFTFSNYGMHWVRQAPGKGLEWVAVISNDGSEKYYADSVKGRFTISRDSSKNTLYLQMNSLRAEDTAVYYCAKAPWGSGTTGRLDYWGQGTLVTVSS

SEQ ID NO: 82 - protein, 121 aa, synthetic construct
EFQLQESGPGLVKPSETLSLTCTVSGGSISTSSYYWGWIRQPPGKGLEWIGTIYYSGTTYYNPSLKSRVTISVDTSKNQFSLKLKSVTAADTSIYYCARHWPKYNSWFDPWGQGILVTVSS

SEQ ID NO: 83 - protein, 121 aa, synthetic construct
EFQLQESGPGLVKPSETLSLTCTVSGGSISTSSYYWGWIRQPPGKGLEWIGTIYFSGTTYYNPSLTSRVSMSVDTSKNQFSLKLNSVTAADTSIYYCARHWPKYNSWFDPWGQGFLVTVSS

SEQ ID NO: 84 - protein, 12 aa, synthetic construct
DRWEGYSGWLDY

SEQ ID NO: 85 - protein, 121 aa, synthetic construct
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRWEGYSGWLDYWGQGTLVTVSS

SEQ ID NO: 86 - protein, 12 aa, synthetic construct
DRYEGYSGWLDY

SEQ ID NO: 87 - protein, 121 aa, synthetic construct
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRYEGYSGWLDYWGQGTLVTVSS

SEQ ID NO: 88 - protein, 12 aa, synthetic construct
DRHEGYSGWLDY

SEQ ID NO: 89 - protein, 121 aa, synthetic construct
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRHEGYSGWLDYWGQGTLVTVSS

SEQ ID NO: 90 - protein, 12 aa, synthetic construct
DRFEGYSGWLDY

SEQ ID NO: 91 - protein, 121 aa, synthetic construct
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRFEGYSGWLDYWGQGTLVTVSS

SEQ ID NO: 92 - protein, 12 aa, synthetic construct
DRNEGYSGWLDY

SEQ ID NO: 93 - protein, 121 aa, synthetic construct
EVQLVESGGGVVQPGRSLRLSCAASGFTFSSHGMHWVRQAPGKGLEWVAVISYDESDKYYADSVKGRFTISRDHSKNTLYLQMNSLRAEDTAVYYCVKDRNEGYSGWLDYWGQGTLVTVSS

SEQ ID NO: 94 - protein, 12 aa, synthetic construct
  VARIANT 3: note = wherein X=W, Y, H, F or N
  VARIANT 4: note = wherein X=E
DRXXGYSGWLDY

SEQ ID NO: 95 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97-101 - skipped (5 entries)

SEQ ID NO: 102 - protein, 4 aa, synthetic construct
GGGS

SEQ ID NO: 103 - protein, 4 aa, synthetic construct
SGGG

SEQ ID NO: 104 - protein, 5 aa, synthetic construct
GGGGS

SEQ ID NO: 105 - protein, 7 aa, synthetic construct
GGGGSGS

SEQ ID NO: 106 - protein, 7 aa, synthetic construct
GGGGSGS

SEQ ID NO: 107 - protein, 8 aa, synthetic construct
GGGGSGGS

SEQ ID NO: 108 - protein, 10 aa, synthetic construct
GGGGSGGGGS

SEQ ID NO: 109 - protein, 15 aa, synthetic construct
GGGGSGGGGSGGGGS

SEQ ID NO: 110 - protein, 16 aa, synthetic construct
AKTTPKLEEGEFSEAR

SEQ ID NO: 111 - protein, 17 aa, synthetic construct
AKTTPKLEEGEFSEARV

SEQ ID NO: 112 - protein, 9 aa, synthetic construct
AKTTPKLGG

SEQ ID NO: 113 - protein, 10 aa, synthetic construct
SAKTTPKLGG

SEQ ID NO: 114 - protein, 17 aa, synthetic construct
AKTTPKLEEGEFSEARV

SEQ ID NO: 115 - protein, 6 aa, synthetic construct
SAKTTP

SEQ ID NO: 116 - protein, 10 aa, synthetic construct
SAKTTPKLGG

SEQ ID NO: 117 - protein, 6 aa, synthetic construct
RADAAP

SEQ ID NO: 118 - protein, 9 aa, synthetic construct
RADAAPTVS

SEQ ID NO: 119 - protein, 12 aa, synthetic construct
RADAAAAGGPGS

SEQ ID NO: 120 - protein, 27 aa, synthetic construct
RADAAAAGGGGSGGGGSGGGGSGGGGS

SEQ ID NO: 121 - protein, 6 aa, synthetic construct
SAKTTP

SEQ ID NO: 122 - protein, 10 aa, synthetic construct
SAKTTPKLGG

SEQ ID NO: 123 - protein, 18 aa, synthetic construct
SAKTTPKLEEGEFSEARV

SEQ ID NO: 124 - protein, 5 aa, synthetic construct
ADAAP

SEQ ID NO: 125 - protein, 12 aa, synthetic construct
ADAAPTVSIFPP

SEQ ID NO: 126 - protein, 5 aa, synthetic construct
TVAAP

SEQ ID NO: 127 - protein, 12 aa, synthetic construct
TVAAPSVFIFPP

SEQ ID NO: 128 - protein, 6 aa, synthetic construct
QPKAAP

SEQ ID NO: 129 - protein, 13 aa, synthetic construct
QPKAAPSVTLFPP

SEQ ID NO: 130 - protein, 6 aa, synthetic construct
AKTTPP

SEQ ID NO: 131 - protein, 13 aa, synthetic construct
AKTTPPSVTPLAP

SEQ ID NO: 132 - protein, 6 aa, synthetic construct
AKTTAP

SEQ ID NO: 133 - protein, 13 aa, synthetic construct
AKTTAPSVYPLAP

SEQ ID NO: 134 - protein, 6 aa, synthetic construct
ASTKGP

SEQ ID NO: 135 - protein, 13 aa, synthetic construct
ASTKGPSVFPLAP

SEQ ID NO: 136 - protein, 15 aa, synthetic construct
GENKVEYAPALMALS

SEQ ID NO: 137 - protein, 15 aa, synthetic construct
GPAKELTPLKEAKVS

SEQ ID NO: 138 - protein, 15 aa, synthetic construct
GHEAAAVMQVQYPAS

SEQ ID NO: 139 - protein, 20 aa, synthetic construct
GGGGSGGGGSGGGGSGGGGS

SEQ ID NO: 140-141 - skipped (2 entries)

SEQ ID NO: 142 - protein, 125 aa, synthetic construct [sequence omitted]

SEQ ID NO: 143 - protein, 107 aa, synthetic construct [sequence omitted]

SEQ ID NO: 144 - protein, 121 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSS

SEQ ID NO: 145 - protein, 121 aa, synthetic construct
EFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSS

SEQ ID NO: 146 - protein, 723 aa, synthetic construct
EFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 147 - protein, 723 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 148 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 451 aa, synthetic construct
EFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 451 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 151 - protein, 486 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 152 - protein, 486 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 723 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 723 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 155 - protein, 5 aa, synthetic construct
TYGMH

SEQ ID NO: 156 - protein, 17 aa, synthetic construct
VIWDDGSYKYYGDSVKG

SEQ ID NO: 157 - protein, 16 aa, synthetic construct
DGITMVRGVMKDYFDY

SEQ ID NO: 158 - protein, 11 aa, synthetic construct
RASQDISSALV

SEQ ID NO: 159 - protein, 7 aa, synthetic construct
DASSLES

SEQ ID NO: 160 - protein, 9 aa, synthetic construct
QQFNSYPLT

SEQ ID NO: 161 - protein, 252 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 162 - DNA, 363 bp, synthetic construct
  source 1..363: mol_type = other DNA
gaagtgcagctggtggagtccgggggcgaggtggtccaacccggggagagcctgagactgagctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagaaacgagggctacagcggctggctggactactggggccaaggcaccctggtgaccgtgagcagc

SEQ ID NO: 163 - DNA, 363 bp, synthetic construct
  source 1..363: mol_type = other DNA
gagtttcagctgcaagagagcggccccggcctggtgaagcctagcgagaccctgagactgacctgcaccgtgagcggcggcagcatcagcacaagcagctactactggggctggatcagacagccccccggcaagggcctggagtggatcggcaccatctactacagcggcaccacctactacaaccctagcctgaagagcagagtgaccatcagcgtggacacaagcaagaatcagttcagcctgaagctgaagagcgtgagagccgccgacacaagcatctactactgcgctagacactggcccaagtacaacagctggttcgacccctggggccaaggcatcctggtgaccgtgagcagc

SEQ ID NO: 164 - DNA, 2169 bp, synthetic construct
  source 1..2169: mol_type = other DNA
gccattcagctgacacagagccctagcagcctgagcgctagcgtgggcgacagagtgacaatcacctgcagagctagccaagacatcagcagcgccctggtgtggtatcagcagaagcccggcaaggcccccaagctgctgatctacgacgctagcagcctcgaaagcggcgtgcctagcagatttagcggcagcgagagcggcaccgacttcaccctgaccatcagcagcctgcagcccgaggacttcgccacctactactgtcagcagttcaacagctaccccctgaccttcggcgggggcacaaaagtggaaattaagggtggaggcggttcaggcggaggtggctctggcggtggcggatcgggtggcggaggctcacaagtgcaactggtcgaaagcggcgggggggtggtgcagcctgggagaagcctgagactgagctgcgccgctagcggcttcaccttcagcacctacggcatgcactgggtgagacaagcccccggcaaaggcctggaatgggtggccgtgatctgggacgacggcagctacaagtactacggcgacagcgtgaagggcagattcaccatcagcagagacaacagcaagaacaccctgtacctgcagatgaacagcctgagagccgaggacaccgccgtgtactactgcgctagagacggcatcaccatggtgagaggcgtgatgaaggactactttgactattgggggcaaggcaccctggtgaccgtgagcagcggtggaggcggttcaggcggaggtggctctggcggtggcggatcgggtggcggaggctcagagtttcagctgcaagagtccggccccggcctggtgaagcctagcgagaccctgagactgacctgcaccgtgagcggcggcagcatcagcacaagcagctactactggggctggatcagacagcctcccggcaagggcctggagtggatcggcaccatctactacagcggcaccacctactacaaccctagcctgaagagcagagtgaccatcagcgtggacacaagcaagaatcagttcagcctgaagctgaagagcgtgagagccgccgacacaagcatctattactgcgcccggcactggcccaagtacaacagctggttcgacccctggggccaaggcatcctggtgaccgtcagcagcgcctccacaaaggggcctagcgtgttccctctggcccctagcagcaaaagcacaagcggcggcaccgctgccctgggctgcctggtcaaggattacttccccgagcccgtgaccgtgagctggaatagcggcgctctcacaagcggcgtgcacaccttccccgccgtgctgcagagcagcggcctgtacagcctgagcagcgtggtgaccgtgcctagcagcagcctgggcacacagacctacatctgcaacgtgaaccacaagcctagcaacaccaaggtggacaaaaaggtggagcccaagtcctgtgacaagacccacacctgccccccctgtcccgcccccgaactcctgggcggccctagcgtgttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagaggagatgaccaagaaccaagtgagcctgtggtgcctggtgaaggggttttaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgagcctgagccccggcaag

SEQ ID NO: 165 - DNA, 1353 bp, synthetic construct
  source 1..1353: mol_type = other DNA
gaggtgcagctggtcgagagcgggggcgaagtggtccaacccggcgagagcctgagactgtcctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagaaacgagggctacagcggctggctggactactggggccaaggcaccctggtgaccgtgtcctccgctagcaccaagggccctagcgtgtttcccctggcccctagcagcaagtccacctccggcgggacagccgctctgggctgcctggtgaaggactacttccccgagcccgtgacagtgagctggaactccggcgctctgacaagcggcgtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaaatcctgcgataaaacccacacatgccccccctgccccgcccccgagctgctgggcggcccctccgtcttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagaggagatgaccaagaaccaagtgagcctgagctgcgccgtgaagggcttctaccctagcgacatcgccgtggagtgggagagcaacgggcagcccgagaacaactacaagaccaccccccccgtgctggacagcgacggcagcttcttcctggtgagcaagctgaccgtggacaagagcagatggcagcaaggcaacgtgttcagctgcagcgtgatgcacgaggccctgcacaaccactacacacagaagagcctgagcctgagccccggcaag

SEQ ID NO: 166 - DNA, 642 bp, synthetic construct
  source 1..642: mol_type = other DNA
gagattgtgctgacacagagccccgccaccctgtccctgtcccccggggagagagccaccctgagctgcagagctagccaaagcgtgagcagctacctggcctggtatcagcagaagcccggccaagcccctagactgctgatctacgacgctagcaacagagccaccggcatccccgctagattcagcggcagcggcagcggcaccgacttcaccctgaccatcagcagcctggagcccgaggacttcgccgtgtactactgtcagcagagaagcaactggccccccaccttcggccaaggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 167 - protein, 1210 aa, synthetic construct [sequence omitted]

SEQ ID NO: 168 - protein, 723 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSEFQLQESGPGLVKPSETLRLTCTVSGGSISTSSYYWGWIRQPPGKGLEWIGTIYYSGTTYYNPSLKSRVTISVDTSKNQFSLKLKSVRAADTSIYYCARHWPKYNSWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 451 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRNEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 170 - protein, 451 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRFEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 171 - protein, 451 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRFEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 172 - protein, 451 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRHEGYSGWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 173 - protein, 121 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRFEGYSGWLDYWGQGTLVTVSS

SEQ ID NO: 174 - protein, 121 aa, synthetic construct
EVQLVESGGEVVQPGESLRLSCAASGFTFSSHGMHWVRQAPGKGLEWVAVISYDESDKYYADSVKGRFTISRDHSKNTLYLEMNSLRAEDTAVYYCVKDRHEGYSGWLDYWGQGTLVTVSS

SEQ ID NO: 175 - DNA, 374 bp, synthetic construct
  source 1..374: mol_type = other DNA
caagtgcaactggtcgaaagcggcgggggggtggtgcagcctgggagaagcctgagactgagctgcgccgctagcggcttcaccttcagcacctacggcatgcactgggtgagacaagcccccggcaaaggcctggaatgggtggccgtgatctgggacgacggcagctacaagtactacggcgacagcgtgaagggcagattcaccatcagcagagacaacagcaagaacaccctgtacctgcagatgaacagcctgagagccgaggacaccgccgtgtactactgcgctagagacggcatcaccatggtgagaggcgtgatgaaggactactttgactattgggggcaaggcaccctggtgaccgtgagcag

SEQ ID NO: 176 - DNA, 321 bp, synthetic construct
  source 1..321: mol_type = other DNA
gccattcagctgacacagagccctagcagcctgagcgctagcgtgggcgacagagtgacaatcacctgcagagctagccaagacatcagcagcgccctggtgtggtatcagcagaagcccggcaaggcccccaagctgctgatctacgacgctagcagcctcgaaagcggcgtgcctagcagatttagcggcagcgagagcggcaccgacttcaccctgaccatcagcagcctgcagcccgaggacttcgccacctactactgtcagcagttcaacagctaccccctgaccttcggcgggggcacaaaagtggaaattaag

SEQ ID NO: 177 - DNA, 363 bp, synthetic construct
  source 1..363: mol_type = other DNA
gagtttcagctgcaagagtccggccccggcctggtgaagcctagcgagaccctgagactgacctgcaccgtgagcggcggcagcatcagcacaagcagctactactggggctggatcagacagcctcccggcaagggcctggagtggatcggcaccatctactacagcggcaccacctactacaaccctagcctgaagagcagagtgaccatcagcgtggacacaagcaagaatcagttcagcctgaagctgaagagcgtgagagccgccgacacaagcatctattactgcgcccggcactggcccaagtacaacagctggttcgacccctggggccaaggcatcctggtgaccgtcagcagc

SEQ ID NO: 178 - DNA, 363 bp, synthetic construct
  source 1..363: mol_type = other DNA
gaggtgcagctggtcgagagcgggggcgaagtggtccaacccggcgagagcctgagactgtcctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagaaacgagggctacagcggctggctggactactggggccaaggcaccctggtgaccgtgtcctcc

SEQ ID NO: 179 - DNA, 321 bp, synthetic construct
  source 1..321: mol_type = other DNA
gagattgtgctgacacagagccccgccaccctgtccctgtcccccggggagagagccaccctgagctgcagagctagccaaagcgtgagcagctacctggcctggtatcagcagaagcccggccaagcccctagactgctgatctacgacgctagcaacagagccaccggcatccccgctagattcagcggcagcggcagcggcaccgacttcaccctgaccatcagcagcctggagcccgaggacttcgccgtgtactactgtcagcagagaagcaactggccccccaccttcggccaaggcaccaaggtggagatcaag

SEQ ID NO: 180 - DNA, 363 bp, synthetic construct
  source 1..363: mol_type = other DNA
gaggtgcagctggtcgagagcgggggcgaagtggtccaacccggcgagagcctgagactgtcctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagattcgagggctacagcggctggctggactactggggccaaggcaccctggtgaccgtgtcctcc

SEQ ID NO: 181 - DNA, 363 bp, synthetic construct
  source 1..363: mol_type = other DNA
gaggtgcagctggtcgagagcgggggcgaagtggtccaacccggcgagagcctgagactgtcctgcgccgctagcggcttcaccttcagcagccacggcatgcactgggtgagacaagcccccggcaagggcctggagtgggtggccgtgatcagctacgacgagagcgacaagtactacgccgacagcgtgaagggcagattcaccatcagcagagaccacagcaagaacaccctgtacctggagatgaacagcctgagagccgaggacaccgccgtgtactactgcgtgaaggacagacacgagggctacagcggctggctggactactggggccaaggcaccctggtgaccgtgtcctcc

SEQ ID NO: 182 - protein, 454 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 446 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 184 - protein, 217 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 185 - protein, 214 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0061067 A1
(43) Pub. Date: Mar. 5, 2026

(54) Anti-cMet and Anti-EGFR Multispecific Antibody Drug Conjugates

(71) Applicant: BeOne Medicines I GmbH, Basel (CH)

(72) Inventors: CHARNG-SHENG TSAI, Beijing (CN); Mei-Hsuan TSAI, Shanghai (CN); Maomao HE, Shanghai (CN); Wei LUO, Shanghai (CN); Zewei WANG, Shanghai (CN); Renke LI, Shanghai (CN); Xiaodong WEI, Shanghai (CN); Ming JIANG, Beijing (CN); Liu XUE, Beijing (CN); Mengran QIAN, Shanghai (CN)

(73) Assignee: BeOne Medicines I GmbH, Basel (CH)

(21) Appl. No.: 19/277,531

(22) Filed: Jul. 23, 2025

(30) Foreign Application Priority Data
Jul. 24, 2024 (WO) PCT/CN2024/107265
May 23, 2025 (WO) PCT/CN2025/096796

(51) Int. Cl. A61K 47/68; A61P 35/00; C07K 16/28

(52) U.S. Cl. CPC A61K 47/68037; A61K 47/6879; A61K 47/6889; A61P 35/00; C07K 16/2863

(57) ABSTRACT

The present disclosure provides antibody drug conjugates comprising antibodies and antigen-binding fragments thereof and a linker-payload, a pharmaceutical composition comprising the antibody drug conjugates, and use of the antibody drug conjugate for treating diseases or disorders. The antibodies or antigen-binding fragments thereof specifically bind to a first and second epitope of human cMET and to human EGFR. The diseases or disorders are EGFR/cMET-associated diseases or disorders.

### CROSS REFERENCE TO RELATED APPLICATIONS

This application claims priority to International Application No. PCT/CN2024/107265, filed Jul. 24, 2024, and to International Application No. PCT/CN2025/096796, filed May 23, 2025, the disclosures of which are hereby incorporated by reference in their entireties.

### FIELD OF THE DISCLOSURE

Disclosed herein are antibody drug conjugates (ADCs) comprising antibodies, or antigen-binding fragments thereof, that bind to human cMET and human EGFR, and are covalently linked to a growth-inhibiting agent, as well as therapeutic uses thereof.

### SEQUENCE LISTING

This application contains a Sequence Listing, which has been submitted electronically in XML format. The XML file is entitled “B240901C-01PCT-sequence listing.xml,” was created on May 27, 2025, and is 189,025 bytes in size. The Sequence Listing is incorporated herein by reference in its entirety.

### BACKGROUND

Antibody drug conjugates (ADCs) are chimeric molecules that combine antibody specificity to recognize and bind with high affinity to antigens such as tumor-associated antigens (TAA) with the potent enzymatic activity of a drug, e.g., toxin, to induce the death of target cells.

The receptor tyrosine kinase mesenchymal-epithelial transition factor (cMET, also known as c-MET, MET, hepatocyte growth factor receptor, or HGFR) and its ligand hepatocyte growth factor (HGF) play roles in multiple cellular processes that stimulate cellular proliferation, survival, invasion, and angiogenesis. Dysregulated HGF-cMET signaling has been observed in multiple solid cancer types including gastric cancer, colorectal cancer, lung cancer, liver cancer, head and neck cancer, breast cancer, and brain cancer. Activating mutations in the kinase domain of epidermal growth factor receptor (EGFR) has also been observed in cancer patients, including a substantial percentage of non-small cell lung cancer (NSCLC) patients.

Potential therapeutics targeting cMET and/or EGFR have shortcomings, including acquired resistance and lack of efficacy. There remains an unmet need for therapeutics targeting cMET and/or EGFR.

### SUMMARY OF THE DISCLOSURE

The present disclosure is directed to antibody drug conjugates (ADCs) comprising an antibody or antigen-binding fragment (Ab) thereof capable of specific binding to human cMET and human EGFR, and a cytotoxic agent (P, PA, or D).

The disclosure contains, inter alia, antibody drug conjugates (ADCs), comprising multispecific antibodies or antigen binding fragments thereof that bind to human cMET and human EGFR. In some embodiments, the multispecific antibodies or antibody fragments thereof comprised within the conjugates specifically bind to human EGFR and two distinct epitopes of human cMET (e.g., non-overlapping epitopes). In some embodiments, the antibody drug conjugates can be used for the treatment of cancer.

The present disclosure encompasses, inter alia, the following embodiments.

Embodiment 1. An antibody drug conjugate, comprising:
a multispecific antibody or antigen-binding fragment thereof, comprising
a first antigen binding domain that specifically binds to a first epitope of human cMET;
a second antigen binding domain that specifically binds to a second epitope of human cMET; and
a third antigen binding domain that specifically binds to human EGFR;
wherein the first epitope is distinct from the second epitope, or wherein the first antigen binding domain does not compete with the second antigen binding domain for binding to cMET;
wherein the antibody drug conjugate has the Formula A:

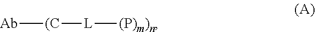

or a pharmaceutically acceptable salt, solvate, or hydrate thereof, wherein:
Ab is the multispecific antibody or antigen-binding fragment thereof;
C is a conjugator;
L is a linker;
P is a payload;
m is an integer from 1 to 8; and
n is from 1 to 15.

Embodiment 2. The antibody drug conjugate of embodiment 1, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 6, (b) a HCDR2 of SEQ ID NO: 7, (c) a HCDR3 of SEQ ID NO: 90 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 6, (b) a HCDR2 of SEQ ID NO: 7, (c) a HCDR3 of SEQ ID NO: 88 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 6, (b) a HCDR2 of SEQ ID NO: 7, (c) a HCDR3 of SEQ ID NO: 86 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68;
(15) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68;
(16) a heavy chain variable region that comprises (a) a HCDR1 of SEQ ID NO: 6, (b) a HCDR2 of SEQ ID NO: 7, (c) a HCDR3 of SEQ ID NO: 84 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 3. The antibody drug conjugate of embodiment 1 or 2, wherein the first antigen binding domain that specifically binds to the first epitope of human cMET comprises:
(1) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 144, SEQ ID NO: 85, SEQ ID NO: 87, SEQ ID NO: 89, SEQ ID NO: 91, or SEQ ID NO: 93, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(2) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 4 or SEQ ID NO: 72, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(3) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 9 or SEQ ID NO: 73, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(4) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 14 or SEQ ID NO: 74, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(5) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 19 or SEQ ID NO: 75, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(6) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 24 or SEQ ID NO: 76, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(7) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 29 or SEQ ID NO: 77, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(8) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 34 or SEQ ID NO: 78, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(9) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 39 or SEQ ID NO: 79, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(10) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 44 or SEQ ID NO: 80, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64; or
(11) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 49 or SEQ ID NO: 81, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64.

Embodiment 4. The antibody drug conjugate of embodiment 2 or 3, wherein the first antigen binding domain that specifically binds to the first epitope of human cMET comprises a heavy chain variable region (VH) comprising the amino acid sequence of SEQ ID NO: 144, SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, or SEQ ID NO: 81, modified in that one, two, three, four, five, six, seven, eight, nine, or ten amino acids have been inserted, deleted or substituted; and/or wherein the first antigen binding domain that specifically binds to the first epitope of human cMET comprises a light chain variable region (VL) comprising the amino acid sequence of SEQ ID NO: 64 modified in that one, two, three, four, five, six, seven, eight, nine, or ten amino acids have been inserted, deleted or substituted.

Embodiment 5. The antibody drug conjugate of any one of embodiments 1-4, wherein the first antigen binding domain that specifically binds to the first epitope of human cMET comprises:
(1) a heavy chain variable region (VH) comprising the amino acid sequence of SEQ ID NO: 144, SEQ ID NO: 85, SEQ ID NO: 87, SEQ ID NO: 89, SEQ ID NO: 91, or SEQ ID NO: 93, and a light chain variable region (VL) comprising the amino acid sequence of SEQ ID NO: 64;
(2) a heavy chain variable region (VH) comprising the amino acid sequence of SEQ ID NO: 4 or SEQ ID NO: 72, and a light chain variable region (VL) comprising the amino acid sequence of SEQ ID NO: 64;
(3) a heavy chain variable region (VH) comprising the amino acid sequence of SEQ ID NO: 9 or SEQ ID NO: 73, and a light chain variable region (VL) comprising the amino acid sequence of SEQ ID NO: 64;
(4) a heavy chain variable region (VH) comprising the amino acid sequence of SEQ ID NO: 14, or SEQ ID NO: 74, and a light chain variable region (VL) comprising the amino acid sequence of SEQ ID NO: 64;
(5) a heavy chain variable region (VH) comprising the amino acid sequence of SEQ ID NO: 19, or SEQ ID NO: 75, and a light chain variable region (VL) comprising the amino acid sequence of SEQ ID NO: 64;
(6) a heavy chain variable region (VH) comprising the amino acid sequence of SEQ ID NO: 24, or SEQ ID NO: 76, and a light chain variable region (VL) of SEQ ID NO: 64;
(7) a heavy chain variable region (VH) comprising the amino acid sequence of SEQ ID NO: 29, or SEQ ID NO: 77, and a light chain variable region (VL) comprising the amino acid sequence of SEQ ID NO: 64;
(8) a heavy chain variable region (VH) comprising the amino acid sequence of SEQ ID NO: 34, or SEQ ID NO: 78, and a light chain variable region (VL) comprising the amino acid sequence of SEQ ID NO: 64;
(9) a heavy chain variable region (VH) comprising the amino acid sequence of SEQ ID NO: 39, or SEQ ID NO: 79, and a light chain variable region (VL) comprising the amino acid sequence of SEQ ID NO: 64;
(10) a heavy chain variable region (VH) comprising the amino acid sequence of SEQ ID NO: 44 or SEQ ID NO: 80, and a light chain variable region (VL) comprising the amino acid sequence of SEQ ID NO: 64; or
(11) a heavy chain variable region (VH) comprising the amino acid sequence of SEQ ID NO: 49 or SEQ ID NO: 81, and a light chain variable region (VL) comprising the amino acid sequence of SEQ ID NO: 64.

Embodiment 6. The antibody drug conjugate of any one of embodiments 1-5, wherein the second antigen binding domain that specifically binds to the second epitope of human cMET comprises:
(1) a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; or
(2) a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68.

Embodiment 7. The antibody drug conjugate of any one of embodiments 1-6, wherein the second antigen binding domain that specifically binds to the second epitope of human cMET comprises:
(1) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 54, or SEQ ID NO: 82, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64;
(2) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 59, or SEQ ID NO: 83, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64; or
(3) a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 145, and a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 64.

Embodiment 8. The antibody drug conjugate of embodiment 6 or embodiment 7, wherein in the second antigen binding domain that specifically binds to the second epitope of human cMET, comprises a heavy chain variable region (VH) comprising the amino acid sequence of SEQ ID NO: 54, SEQ ID NO: 82, SEQ ID NO: 59, SEQ ID NO: 83, or SEQ ID NO: 145 modified in that one, two, three, four, five, six, seven, eight, nine, or ten amino acids have been inserted, deleted or substituted; and/or wherein the second antigen binding domain that specifically binds to the second epitope of human cMET comprises a light chain variable region (VL) comprising the amino acid sequence of SEQ ID NO: 64 modified in that one, two, three, four, five, six, seven, eight, nine, or ten amino acids have been inserted, deleted or substituted.

Embodiment 9. The antibody drug conjugate of any one of embodiments 1-8, wherein the second antigen binding domain that specifically binds to the second epitope of human cMET comprises:
(1) a heavy chain variable region (VH) comprising the amino acid sequence of SEQ ID NO: 54, or SEQ ID NO: 82, and a light chain variable region (VL) comprising the amino acid sequence of SEQ ID NO: 64;
(2) a heavy chain variable region (VH) comprising the amino acid sequence of SEQ ID NO: 59, or SEQ ID NO: 83, and a light chain variable region (VL) comprising the amino acid sequence of SEQ ID NO: 64; or
(3) a heavy chain variable region (VH) comprising the amino acid sequence of SEQ ID NO: 145, and a light chain variable region (VL) comprising the amino acid sequence of SEQ ID NO: 64.

Embodiment 10. The antibody drug conjugate of any one of embodiments 1-9, wherein the third antigen binding domain that specifically binds to human EGFR comprises:
a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 11. The antibody drug conjugate of any one of embodiments 1-10, wherein the third antigen binding domain that specifically binds to human EGFR comprises a heavy chain variable region (VH) comprising an amino acid sequence at least 90%, at least 91%, at least 92%, at least 93%, at least 94%, at least 95%, at least 96%, at least 97%, at least 98% or at least 99% identical to SEQ ID NO: 142, and/or a light chain variable region (VL) comprising an amino acid sequence at least 90%, at least 91%, at least 92%, at least 93%, at least 94%, at least 95%, at least 96%, at least 97%, at least 98% or at least 99% identical to SEQ ID NO: 143.

Embodiment 12. The antibody drug conjugate of embodiment 6 or embodiment 7, wherein the third antigen binding domain that specifically binds to human EGFR comprises a heavy chain variable region (VH) comprising the amino acid sequence of SEQ ID NO: 142 modified in that one, two, three, four, five, six, seven, eight, nine, or ten amino acids have been inserted, deleted or substituted; and/or wherein the third antigen binding domain that specifically binds to human EGFR comprises a light chain variable region (VL) comprising the amino acid sequence of SEQ ID NO: 143 modified in that one, two, three, four, five, six, seven, eight, nine, or ten amino acids have been inserted, deleted or substituted.

Embodiment 13. The antibody drug conjugate of any one of embodiments 1-12, wherein the third antigen binding domain that specifically binds to human EGFR comprises a heavy chain variable region (VH) comprising the amino acid sequence of SEQ ID NO: 142, and/or a light chain variable region (VL) comprising the amino acid sequence of SEQ ID NO: 143.

Embodiment 14. The antibody drug conjugate of any one of embodiments 1-13, wherein
the first antigen binding domain that specifically binds to the first epitope of human cMET comprises:
a heavy chain variable region (VH) that comprises (a) a HCDR1 of SEQ ID NO: 6, (b) a HCDR2 of SEQ ID NO: 7, (c) a HCDR3 of SEQ ID NO: 94, 84, 86, 88, 90, or 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises:
a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises:
a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 15. The antibody drug conjugate of any one of embodiments 1-13, wherein
the first antigen binding domain that specifically binds to the first epitope of human cMET comprises:
a heavy chain variable region (VH) comprising the amino acid sequence of SEQ ID NO: 144, SEQ ID NO: 85, SEQ ID NO: 87, SEQ ID NO: 89, SEQ ID NO: 91, or SEQ ID NO: 93, and a light chain variable region (VL) comprising the amino acid sequence of SEQ ID NO: 64; the second antigen binding domain that specifically binds to the second epitope of human cMET comprises:
a heavy chain variable region (VH) comprising the amino acid sequence of SEQ ID NO: 145, SEQ ID NO: 54, or SEQ ID NO: 82, and a light chain variable region (VL) comprising the amino acid sequence of SEQ ID NO: 64; and
the third antigen binding domain that specifically binds to human EGFR comprises:
a heavy chain variable region (VH) comprising the amino acid sequence of SEQ ID NO: 142, and a light chain variable region (VL) comprising the amino acid sequence of SEQ ID NO: 143.

Embodiment 16. The antibody drug conjugate of any one of embodiments 1-13, wherein
the first antigen binding domain that specifically binds to the first epitope of human cMET comprises:
(1) a heavy chain variable region (VH) that comprises (a) a HCDR1 of SEQ ID NO: 6, (b) a HCDR2 of SEQ ID NO: 7, (c) a HCDR3 of SEQ ID NO: 94, 84, 86, 88, 90, or 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises:
a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises:
a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 17. The antibody drug conjugate of any one of embodiments 1-16, wherein
the first antigen binding domain that specifically binds to the first epitope of human cMET comprises:
a heavy chain variable region (VH) comprising the amino acid sequence of SEQ ID NO: 144, SEQ ID NO: 85, SEQ ID NO: 87, SEQ ID NO: 89, SEQ ID NO: 91, or SEQ ID NO: 93, and a light chain variable region (VL) comprising the amino acid sequence of SEQ ID NO: 64;
the second antigen binding domain that specifically binds to the second epitope of human cMET comprises:
a heavy chain variable region (VH) comprising the amino acid sequence of SEQ ID NO: 59, or SEQ ID NO: 83, and a light chain variable region (VL) comprising the amino acid sequence of SEQ ID NO: 64; and
the third antigen binding domain that specifically binds to human EGFR comprises:
a heavy chain variable region (VH) comprising the amino acid sequence of SEQ ID NO: 142, and a light chain variable region (VL) comprising the amino acid sequence of SEQ ID NO: 143.

Embodiment 18. The antibody drug conjugate of any one of embodiments 1-17, wherein
the first antigen binding domain that specifically binds to the first epitope of human cMET comprises: a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises: a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises: a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

Embodiment 19. The antibody drug conjugate of any one of embodiments 1-18, wherein
the first antigen binding domain that specifically binds to the first epitope of human cMET comprises: a heavy chain variable region (VH) comprising the amino acid sequence of SEQ ID NO: 144, and a light chain variable region (VL) comprising the amino acid sequence of SEQ ID NO: 64;
the second antigen binding domain that specifically binds to the second epitope of human cMET comprises: a heavy chain variable region (VH) comprising the amino acid sequence of SEQ ID NO: 145, and a light chain variable region (VL) comprising the amino acid sequence of SEQ ID NO: 64; and
the third antigen binding domain that specifically binds to human EGFR comprises: a heavy chain variable region (VH) comprising the amino acid sequence of SEQ ID NO: 142, and a light chain variable region (VL) comprising the amino acid sequence of SEQ ID NO: 143.

Embodiment 20. The antibody drug conjugate of any one of embodiments 1-19, wherein the first antigen binding domain that specifically binds to the first epitope of human cMET is a monoclonal antibody, a human engineered antibody, a single chain antibody (scFv), a Fab fragment, a Fab′ fragment, or a F(ab′)2 fragment;
the second antigen binding domain that specifically binds to the second epitope of human cMET is a monoclonal antibody, a human engineered antibody, a single chain antibody (scFv), a Fab fragment, a Fab′ fragment, or a F(ab′)2 fragment; and
the third antigen binding domain that specifically binds to human EGFR is a monoclonal antibody, a human engineered antibody, a single chain antibody (scFv), a Fab fragment, a Fab′ fragment, or a F(ab′)2 fragment.

Embodiment 21. The antibody drug conjugate of any one of embodiments 1-20, wherein the first antigen binding domain that specifically binds to the first epitope of human cMET and the second antigen binding domain that specifically binds to the second epitope of human cMET collectively comprise a bispecific IgG antibody.

Embodiment 22. The antibody drug conjugate of any one of embodiments 1-21, wherein the third antigen binding domain that specifically binds to human EGFR comprises a single chain antibody (scFv), a Fab fragment, a Fab′ fragment, or a F(ab′)2 fragment.

Embodiment 23. The antibody drug conjugate of any one of embodiments 1-22, wherein the first antigen binding domain that specifically binds to the first epitope of human cMET and the second antigen binding domain that specifically binds to the second epitope of human cMET comprise a bispecific IgG antibody, and wherein the third antigen binding domain that specifically binds to human EGFR comprises a single chain Fv (scFv), optionally wherein
(a) the scFv is attached to the C-terminal of a light chain of one or both of the first and second antigen binding domains;
(b) the scFv is attached to the N-terminal of a light chain of one or both of the first and second antigen binding domains;
(c) the scFv is attached to the N-terminal of a heavy chain of both of the first and second antigen binding domains;
(d) the scFv is attached to the N-terminal of a heavy chain of one of the first and second antigen binding domains;
(e) the scFv is attached to the C-terminal of a heavy chain constant region of one of the first and second antigen binding domains; or
(f) the scFv is attached to the C-terminal of a heavy chain constant region of both of the first and second antigen binding domains.

Embodiment 24. The antibody drug conjugate of any one of embodiments 1-23, wherein the third antigen binding domain that specifically binds to human EGFR comprises a scFv comprising a VH comprising the amino acid sequence of SEQ ID NO: 142 and a VL comprising the amino acid sequence of SEQ ID NO: 143;
optionally wherein the VH and VL are connected via a first amino acid linker;
optionally wherein the first amino acid linker comprises the amino acid sequence of any one of SEQ ID NOs: 97-139.

Embodiment 25. The antibody drug conjugate of embodiment 24, wherein the scFv comprises a first amino acid linker comprising the sequence of SEQ ID NO: 139.

Embodiment 26. The antibody drug conjugate of any one of embodiments 1-25, wherein the third antigen binding domain that specifically binds to human EGFR comprises a scFv comprising the amino acid sequence of SEQ ID NO: 161.

Embodiment 27. The antibody drug conjugate of any one of embodiments 1-26, wherein the first antigen binding domain that specifically binds to the first epitope of human cMET and the second antigen binding domain that specifically binds to the second epitope of human cMET collectively comprise a bispecific IgG antibody that comprises a human IgG1, IgG2, IgG3, or IgG4 heavy chain constant region, and/or a human kappa or lambda light chain constant region, wherein the heavy chain constant region optionally lacks a C-terminal lysine or a C-terminal glycine-lysine.

Embodiment 28. The antibody drug conjugate of embodiment 27, wherein the multispecific antibody or antigen-binding fragment thereof comprises a human IgG1 heavy chain constant region and a human kappa light chain constant region.

Embodiment 29. The antibody drug conjugate of any one of embodiments 1-28, wherein the multispecific antibody or antigen-binding fragment thereof has antibody dependent cellular cytotoxicity (ADCC), antibody-dependent cellular phagocytosis (ADCP) or complement dependent cytotoxicity (CDC).

Embodiment 30. The antibody drug conjugate of any one of embodiments 1-29, wherein the multispecific antibody or antigen-binding fragment thereof has reduced glycosylation or no glycosylation or is hypofucosylated.

Embodiment 31. The antibody drug conjugate of any one of embodiments 1-30, wherein the multispecific antibody or antigen-binding fragment thereof is afucosylated.

Embodiment 32. The antibody drug conjugate of any one of embodiments 1-31, wherein the multispecific antibody or antigen-binding fragment thereof comprises increased bisecting GlcNac structures.

Embodiment 33. The antibody drug conjugate of any one of embodiments 1-32, wherein the first antigen binding domain that specifically binds to the first epitope of human cMET and/or the second antigen binding domain that specifically binds to the second epitope of human cMET comprises a human IgG1 heavy chain constant region comprising M252Y, S254T and T256E substitutions (EU numbering), optionally wherein the multispecific antibody or antigen binding fragment thereof has increased half life compared to a multispecific antibody or antigen binding fragment thereof comprising wild-type human IgG1 heavy chain constant regions.

Embodiment 34. The antibody drug conjugate of any one of embodiments 1-33, wherein the first antigen binding domain that specifically binds to the first epitope of human cMET comprises a human IgG1 heavy chain constant region comprising a knob substitution, and the second antigen binding domain that specifically binds to the second epitope of human cMET comprises a human IgG1 heavy chain constant region comprising a hole substitution, optionally wherein the knob substitution is a T366W substitution and/or the hole substitution is at least one of a T366S, L368A, or Y407V substitution (EU numbering); or
wherein the first antigen binding domain that specifically binds to the first epitope of human cMET comprises a human IgG1 heavy chain constant region comprising a hole substitution, and the second antigen binding domain that specifically binds to the second epitope of human cMET comprises a human IgG1 heavy chain constant region comprising a knob substitution, optionally wherein the hole substitution is at least one of a T366S, L368A, or Y407V substitution, and/or the knob substitution is a T366W substitution (EU numbering).

Embodiment 35. The antibody drug conjugate of any one of embodiments 1-33, wherein the first antigen binding domain that specifically binds to the first epitope of human cMET comprises a first heavy chain constant region comprising SEQ ID NO: 95, and the second antigen binding domain that specifically binds to the second epitope of human cMET comprises a second heavy chain constant region comprising SEQ ID NO: 96; or
wherein the first antigen binding domain that specifically binds to the first epitope of human cMET comprises a first heavy chain constant region comprising SEQ ID NO: 96, and the second antigen binding domain that specifically binds to the second epitope of human cMET comprises a second heavy chain constant region comprising SEQ ID NO: 95,
optionally wherein the first and/or the second heavy chain constant region lacks the C-terminal lysine or the C-terminal glycine-lysine.

Embodiment 36. The antibody drug conjugate of any one of embodiments 1-35, wherein the first antigen binding domain that specifically binds to the first epitope of human cMET comprises a first light chain constant region, and the second antigen binding domain that specifically binds to the second epitope of human cMET comprises a second light chain constant region;
optionally wherein the first light chain constant region and the second light chain constant region are different, or wherein the first light chain constant region and the second light chain constant region are the same.

Embodiment 37. The antibody drug conjugate of any one of embodiments 1-36, comprising a first polypeptide, a second polypeptide, a third polypeptide, and a fourth polypeptide and optionally at least one amino acid linker, wherein
(1) the first polypeptide comprises from N-terminal to C-terminal: a VL of the third antigen binding domain that specifically binds to human EGFR, optionally a first amino acid linker, a VH of the third antigen binding domain that specifically binds to human EGFR, optionally a second amino acid linker, a VH of the second antigen binding domain that specifically binds to the second epitope of human cMET, and the second heavy chain constant region;
(2) the second polypeptide comprises from N-terminal to C-terminal: a VH of the first antigen binding domain that specifically binds to the first epitope of human cMET, and a first heavy chain constant region, or
a VL of the third antigen binding domain that specifically binds to human EGFR, optionally a first amino acid linker, a VH of the third antigen binding domain that specifically binds to human EGFR, optionally a second amino acid linker, a VH of the first antigen binding domain that specifically binds to the first epitope of human cMET, and a first heavy chain constant region;
(3) the third polypeptide comprises in the direction of N terminal to C terminal: a VL of the second antigen binding domain that specifically binds to the second epitope of human cMET, and a second light chain constant region; and
(4) the fourth polypeptide comprises in the direction of N terminal to C terminal a VL of the first antigen binding domain that specifically binds to the first epitope of human cMET, and a first light chain constant region;
optionally wherein the first and second heavy chain constant regions comprise human IgG1 constant regions with or without a C-terminal lysine or C-terminal glycine-lysine; optionally wherein the third antigen binding domain that specifically binds to human EGFR is an scFv;
optionally wherein the second heavy chain constant region comprises the amino acid sequence of SEQ ID NO: 95, and the first heavy chain constant region comprises the amino acid sequence of SEQ ID NO: 96; or the second heavy chain constant region comprises the amino acid sequence of SEQ ID NO: 96, and the first heavy chain constant region comprises the amino acid sequence of SEQ ID NO: 95;
optionally wherein the first amino acid linker comprises the amino acid sequence of SEQ ID NO: 139;
optionally wherein the second amino acid linker comprises the amino acid sequence of SEQ ID NO: 139;
optionally wherein the VL of the first antigen binding domain and the VL of the second antigen binding domain are the same;
optionally wherein the first light chain constant region and the second light chain constant region are the same; and
optionally wherein the third polypeptide and the fourth polypeptide are the same.

Embodiment 38. The antibody drug conjugate of any one of embodiments 1-36, comprising a first polypeptide, a second polypeptide, a third polypeptide, and a fourth polypeptide and optionally at least one amino acid linker, wherein
(1) the first polypeptide comprises in the direction of N terminal to C terminal: a VL of the third antigen binding domain that specifically binds to human EGFR, optionally a first amino acid linker, a VH of the third antigen binding domain that specifically binds to human EGFR, optionally a second amino acid linker, a VH of the first antigen binding domain that specifically binds to the first epitope of human cMET, and a first heavy chain constant region;
(2) the second polypeptide comprises in the direction of N terminal to C terminal: a VH of the second antigen binding domain that specifically binds to the second epitope of human cMET, and a second heavy chain constant region, or
the second polypeptide comprises in the direction of N terminal to C terminal: a VL of the third antigen binding domain that specifically binds to human EGFR, optionally a first amino acid linker, a VH of the third antigen binding domain that specifically binds to human EGFR, optionally a second amino acid linker, a VH of the second antigen binding domain that specifically binds to the second epitope of human cMET, and a second heavy chain constant region;
(3) the third polypeptide comprises in the direction of N terminal to C terminal: a VL of the first antigen binding domain that specifically binds to the first epitope of human cMET, and a first light chain constant region; and
(4) the fourth polypeptide comprises in the direction of N terminal to C terminal a VL of the second antigen binding domain that specifically binds to the second epitope of human cMET, and a second light chain constant region;
optionally wherein the first and second heavy chain constant regions comprise human IgG1 constant regions with or without a C-terminal lysine or C-terminal glycine-lysine;
optionally wherein the third antigen binding domain that specifically binds to human EGFR is an scFv;
optionally wherein the second heavy chain constant region comprises the amino acid sequence of SEQ ID NO: 95, and the first heavy chain constant region comprises the amino acid sequence of SEQ ID NO: 96; or the second heavy chain constant region comprises the amino acid sequence of SEQ ID NO: 96, and the first heavy chain constant region comprises the amino acid sequence of SEQ ID NO: 95;
optionally wherein the first amino acid linker comprises the amino acid sequence of SEQ ID NO: 139;
optionally wherein the second amino acid linker comprises the amino acid sequence of SEQ ID NO: 139;
optionally wherein the VL of the first antigen binding domain and the VL of the second antigen binding domain are the same;
optionally wherein the first light chain constant region and the second light chain constant region are the same; and
optionally wherein the third polypeptide and the fourth polypeptide are the same.

Embodiment 39. The antibody drug conjugate of any one of embodiments 1-36, comprising a first polypeptide, a second polypeptide, a third polypeptide, and a fourth polypeptide and optionally at least one amino acid linker, wherein
(1) the first polypeptide comprises in the direction of N terminal to C terminal: a VL of the third antigen binding domain that specifically binds to human EGFR, optionally a first amino acid linker, a VH of the third antigen binding domain that specifically binds to human EGFR, optionally a second amino acid linker, a VH of the second antigen binding domain that specifically binds to the second epitope of human cMET, and a second heavy chain constant region;
(2) the second polypeptide comprises in the direction of N terminal to C terminal: a VH of the first antigen binding domain that specifically binds to the first epitope of human cMET and the first heavy chain constant region;
(3) the third polypeptide comprises in the direction of N terminal to C terminal: a VL of the second antigen binding domain that specifically binds to the second epitope of human cMET and a second light chain constant region; and
(4) the fourth polypeptide comprising in the direction of N terminal to C terminal: a VL of the first antigen binding domain that specifically binds to the first epitope of human cMET and a first light chain constant region;
optionally wherein the first and second heavy chain constant regions comprise human IgG1 constant regions with or without a C-terminal lysine or C-terminal glycine-lysine; optionally wherein the third antigen binding domain that specifically binds to human EGFR is an scFv;
optionally wherein the second heavy chain constant region comprises the amino acid sequence of SEQ ID NO: 95, and the first heavy chain constant region comprises the amino acid sequence of SEQ ID NO: 96; or wherein the second heavy chain constant region comprises the amino acid sequence of SEQ ID NO: 96, and the first heavy chain constant region comprises the amino acid sequence of SEQ ID NO: 95;
optionally wherein the first amino acid linker comprises the amino acid sequence of SEQ ID NO: 139;
optionally wherein the second amino acid linker comprises the amino acid sequence of SEQ ID NO: 139;
optionally wherein a VL of the first antigen binding domain and a VL of the second antigen binding domain are the same;
optionally wherein the first light chain constant region and the second light chain constant region are the same; and
optionally wherein the third polypeptide and the fourth polypeptide are the same.

Embodiment 40. The antibody drug conjugate of any one of embodiments 1-36, comprising a first polypeptide, a second polypeptide, and a third polypeptide, wherein
(1) the first polypeptide comprises the amino acid sequence of SEQ ID NO: 153, the second polypeptide comprises the amino acid sequence of SEQ ID NO: 150, and the third polypeptide comprises the amino acid sequence of SEQ ID NO: 148;
(2) the first polypeptide comprises the amino acid sequence of SEQ ID NO: 146, the second polypeptide comprises the amino acid sequence of SEQ ID NO: 147, and the third polypeptide comprises the amino acid sequence of SEQ ID NO: 148;
(3) the first polypeptide comprises the amino acid sequence of SEQ ID NO: 149, the second polypeptide comprises the amino acid sequence of SEQ ID NO: 150, and the third polypeptide comprises the amino acid sequence of SEQ ID NO: 151;
(4) the first polypeptide comprises the amino acid sequence of SEQ ID NO: 149, the second polypeptide comprises the amino acid sequence of SEQ ID NO: 150, and the third polypeptide comprises the amino acid sequence of SEQ ID NO: 152;
(5) the first polypeptide comprises the amino acid sequence of SEQ ID NO: 153, the second polypeptide comprises the amino acid sequence of SEQ ID NO: 154, and the third polypeptide comprises the amino acid sequence of SEQ ID NO: 148;
(6) the first polypeptide comprises the amino acid sequence of SEQ ID NO: 146, the second polypeptide comprises the amino acid sequence of SEQ ID NO: 150, and the third polypeptide comprises the amino acid sequence of SEQ ID NO: 148;
(7) the first polypeptide comprises the amino acid sequence of SEQ ID NO: 168, the second polypeptide comprises the amino acid sequence of SEQ ID NO: 169, and the third polypeptide comprises the amino acid sequence of SEQ ID NO: 148;
(8) the first polypeptide comprises the amino acid sequence of SEQ ID NO: 153, the second polypeptide comprises the amino acid sequence of SEQ ID NO: 170, and the third polypeptide comprises the amino acid sequence of SEQ ID NO: 148;
(9) the first polypeptide comprises the amino acid sequence of SEQ ID NO: 168, the second polypeptide comprises the amino acid sequence of SEQ ID NO: 171, and the third polypeptide comprises the amino acid sequence of SEQ ID NO: 148; or
(10) the first polypeptide comprises the amino acid sequence of SEQ ID NO: 153, the second polypeptide comprises the amino acid sequence of SEQ ID NO: 172, and the third polypeptide comprises the amino acid sequence of SEQ ID NO: 148.

Embodiment 41. The antibody drug conjugate of any one of embodiments 1-36, wherein the multispecific antibody or antigen-binding fragment thereof comprises
(1) a first polypeptide comprising the amino acid sequence of SEQ ID NO: 153, a second polypeptide comprising the amino acid sequence of SEQ ID NO: 150, and a third polypeptide of SEQ ID NO: 148;
(2) a first polypeptide comprising the amino acid sequence of SEQ ID NO: 146, a second polypeptide comprising the amino acid sequence of SEQ ID NO: 147, and the third polypeptide of SEQ ID NO: 148;
(3) a first polypeptide comprising the amino acid sequence of SEQ ID NO: 149, a second polypeptide comprising the amino acid sequence of SEQ ID NO: 150, and the third polypeptide of SEQ ID NO: 151;
(4) a first polypeptide comprising the amino acid sequence of SEQ ID NO: 149, a second polypeptide comprising the amino acid sequence of SEQ ID NO: 150, and the third polypeptide of SEQ ID NO: 152;
(5) a first polypeptide comprising the amino acid sequence of SEQ ID NO: 153, a second polypeptide comprising the amino acid sequence of SEQ ID NO: 154, and the third polypeptide of SEQ ID NO: 148;
(6) a first polypeptide comprising the amino acid sequence of SEQ ID NO: 146, a second polypeptide comprising the amino acid sequence of SEQ ID NO: 150, and the third polypeptide of SEQ ID NO: 148;
(7) a first polypeptide comprising the amino acid sequence of SEQ ID NO: 168, a second polypeptide comprising the amino acid sequence of SEQ ID NO: 169, and the third polypeptide has the amino acid sequence of SEQ ID NO: 148;
(8) a first polypeptide comprising the amino acid sequence of SEQ ID NO: 153, a second polypeptide comprising the amino acid sequence of SEQ ID NO: 170, and the third polypeptide has the amino acid sequence of SEQ ID NO: 148;
(9) a first polypeptide comprising the amino acid sequence of SEQ ID NO: 168, a second polypeptide comprising the amino acid sequence of SEQ ID NO: 171, and the third polypeptide has the amino acid sequence of SEQ ID NO: 148; or
(10) a first polypeptide comprising the amino acid sequence of SEQ ID NO: 153, a second polypeptide comprising the amino acid sequence of SEQ ID NO: 172, and the third polypeptide has the amino acid sequence of SEQ ID NO: 148.

Embodiment 42. The antibody drug conjugate of any one of embodiments 1-36, comprising a first polypeptide, a second polypeptide, and a third polypeptide, wherein:
(1) the first polypeptide comprises the amino acid sequence of SEQ ID NO: 153, the second polypeptide comprises the amino acid sequence of SEQ ID NO: 150, and the third polypeptide comprises the amino acid sequence of SEQ ID NO: 148;
(7) the first polypeptide comprises the amino acid sequence of SEQ ID NO: 168, the second polypeptide comprises the amino acid sequence of SEQ ID NO: 169, and the third polypeptide comprises the amino acid sequence of SEQ ID NO: 148;
(8) the first polypeptide comprises the amino acid sequence of SEQ ID NO: 153, the second polypeptide comprises the amino acid sequence of SEQ ID NO: 170, and the third polypeptide comprises the amino acid sequence of SEQ ID NO: 148;
(9) the first polypeptide comprises the amino acid sequence of SEQ ID NO: 168, the second polypeptide comprises the amino acid sequence of SEQ ID NO: 171, and the third polypeptide comprises the amino acid sequence of SEQ ID NO: 148; or
(10) the first polypeptide comprises the amino acid sequence of SEQ ID NO: 153, the second polypeptide comprises the amino acid sequence of SEQ ID NO: 172, and the third polypeptide comprises the amino acid sequence of SEQ ID NO: 148.

Embodiment 43. The antibody drug conjugate of any one of embodiments 1-36, wherein the multispecific antibody or antigen-binding fragment thereof comprises
(1) a first polypeptide comprising the amino acid sequence of SEQ ID NO: 153, a second polypeptide comprising the amino acid sequence of SEQ ID NO: 150, and a third polypeptide comprising the amino acid sequence of SEQ ID NO: 148;
(7) a first polypeptide comprising the amino acid sequence of SEQ ID NO: 168, a second polypeptide comprising the amino acid sequence of SEQ ID NO: 169, and a third polypeptide comprising the amino acid sequence of SEQ ID NO: 148;
(8) a first polypeptide comprising the amino acid sequence of SEQ ID NO: 153, a second polypeptide comprising the amino acid sequence e of SEQ ID NO: 170, and a third polypeptide comprising the amino acid sequence of SEQ ID NO: 148;
(9) a first polypeptide comprising the amino acid sequence of SEQ ID NO: 168, a second polypeptide comprising the amino acid sequence of SEQ ID NO: 171, and a third polypeptide comprising the amino acid sequence of SEQ ID NO: 148; or
(10) a first polypeptide comprising the amino acid sequence of SEQ ID NO: 153, a second polypeptide comprising the amino acid sequence of SEQ ID NO: 172, and a third polypeptide comprising the amino acid sequence of SEQ ID NO: 148.

Embodiment 44. The antibody drug conjugate of any one of embodiments 1-43, wherein m is 1.

Embodiment 45. The antibody drug conjugate of any one of embodiments 1-44, wherein the antibody drug conjugate has Formula (A-I):

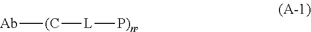

or a pharmaceutically acceptable salt, solvate, or hydrate thereof.

Embodiment 46. The antibody drug conjugate of any one of embodiments 1-45, wherein the antibody drug conjugate has Formula (I):

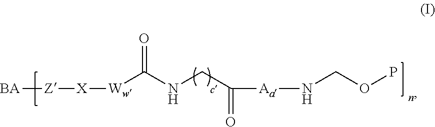

or a pharmaceutically acceptable salt, tautomer, solvate, or stereoisomer thereof, wherein:
BA is Ab;
Z′ is

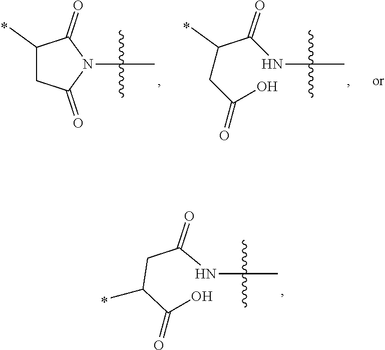

wherein * indicates the bond where Z′ attaches to BA;
X is a substituted or unsubstituted C₁₋₅ alkyl;
W is oxygen;
w′ is 0 or 1;
c′ is 1 or 2;
a′ is 1, 2, or 3;
each A is, independently, an amino acid residue optionally substituted with a hydrophilic group;
P is a payload residue; and
n is from 1 to 15.

Embodiment 47. The antibody drug conjugate of embodiment 46, wherein X is —(CH₂)₃—, —(CH₂)₅—, —CH(CH₂NH₂)(CH₂)₂—, —CH(CH₂NH₂)(CH₂)₄—, —CH₂CH(NH₂)CH₂—, —CH(CH₂NH₂)CH₂—, or —CH(CH₂NH₂)(CH₂)₂—.

Embodiment 48. The antibody drug conjugate of embodiment 46, wherein X is —(CH₂)₅— or —CH(CH₂NH₂)(CH₂)₂—.

Embodiment 49. The antibody drug conjugate of any one of embodiments 46-48, wherein each A is, independently, an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

Embodiment 50. The antibody drug conjugate of embodiment 49, wherein each A is, independently, a glycine, phenylalanine, serine, or valine residue.

Embodiment 51. The antibody drug conjugate of any one of embodiments 46-48, wherein A is substituted with a hydrophilic group (HG), and HG is selected from a saccharide, phosphate ester, sulfate ester, a phosphodiester, and a phosphonate.

Embodiment 52. The antibody drug conjugate of embodiment 51, wherein the hydrophilic group (HG) is:

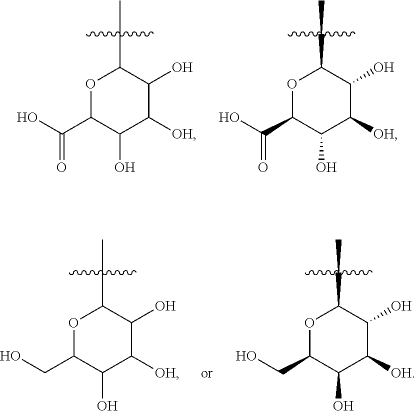 [SMILES: *C1OC(C(=O)O)C(O)C(O)C1O ; *[C@@H]1O[C@H](C(=O)O)[C@@H](O)[C@H](O)[C@H]1O ; *C1OC(CO)C(O)C(O)C1O ; *[C@@H]1O[C@H](CO)[C@H](O)[C@H](O)[C@H]1O]

Embodiment 53. The antibody drug conjugate of any one of embodiments 46-48, wherein A is

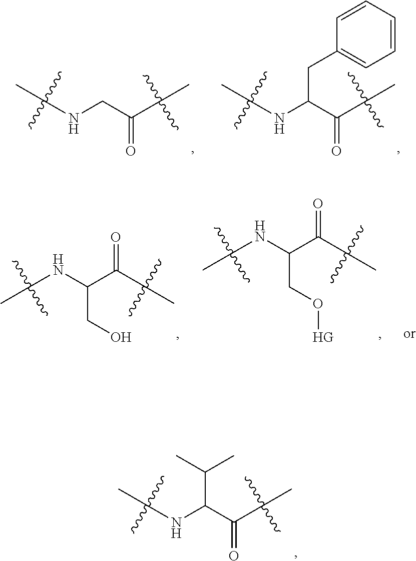

wherein HG is a hydrophilic group.

Embodiment 54. The antibody drug conjugate of embodiment 53, wherein A and HG together are

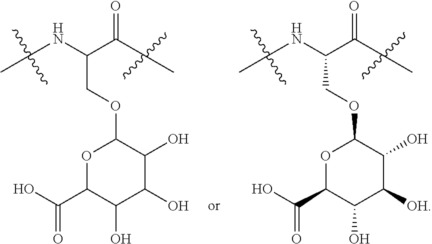 [SMILES: *NC(COC1OC(C(=O)O)C(O)C(O)C1O)C(*)=O ; "or" ; *N[C@@H](CO[C@@H]1O[C@H](C(=O)O)[C@@H](O)[C@H](O)[C@H]1O)C(*)=O]

Embodiment 55. The antibody drug conjugate of embodiment 53, wherein A is

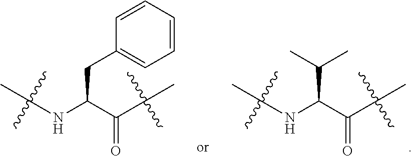

Embodiment 56. The antibody drug conjugate of any one of embodiments 46-48, wherein A is

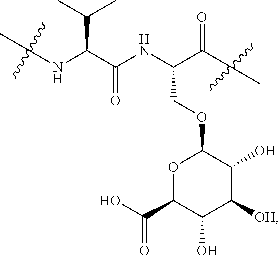 [SMILES: *N[C@H](C(=O)N[C@@H](CO[C@@H]1O[C@H](C(=O)O)[C@@H](O)[C@H](O)[C@H]1O)C(*)=O)C(C)C]

and wherein a′ is 1.

Embodiment 57. The antibody drug conjugate of any one of the embodiments 46-48, wherein A is

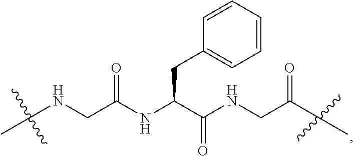 [SMILES: *NCC(=O)N[C@@H](Cc1ccccc1)C(=O)NCC(*)=O]

Embodiment 58. The antibody drug conjugate of any one of embodiments 1-57, wherein P is a cytotoxic agent.

Embodiment 59. The antibody drug conjugate of any one of embodiments 1-58, wherein P is an exatecan analogue.

Embodiment 60. The antibody drug conjugate of any one of embodiments 1-59, wherein P is:

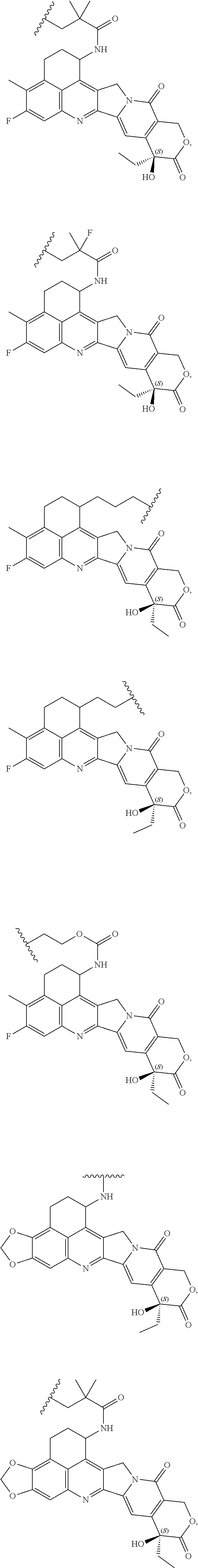

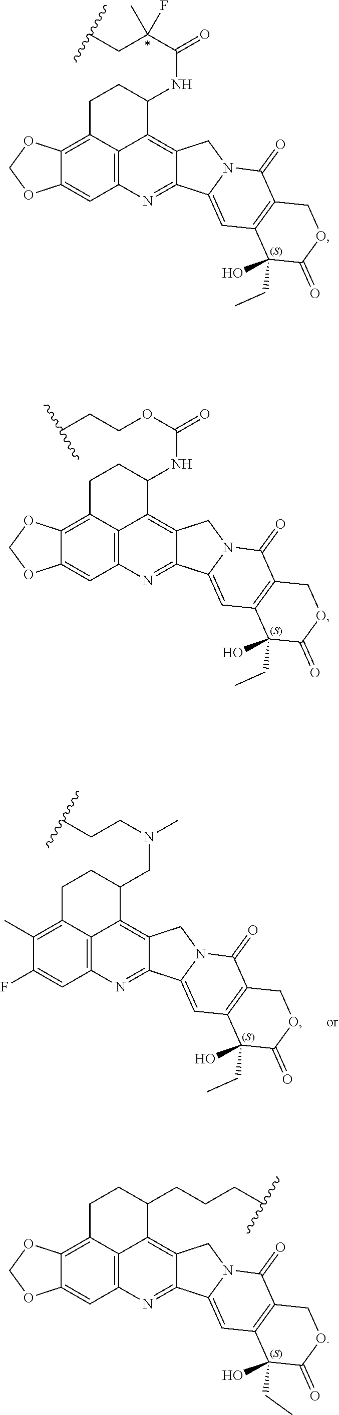

Embodiment 61. The antibody drug conjugate of embodiment 60, wherein P is

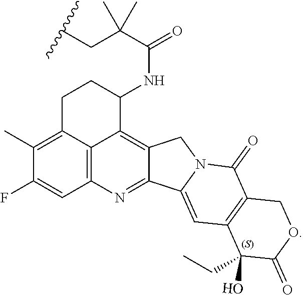

Embodiment 62. The antibody drug conjugate of any one of embodiments 1-44, wherein the antibody drug conjugate is represented by one of the following formulas:

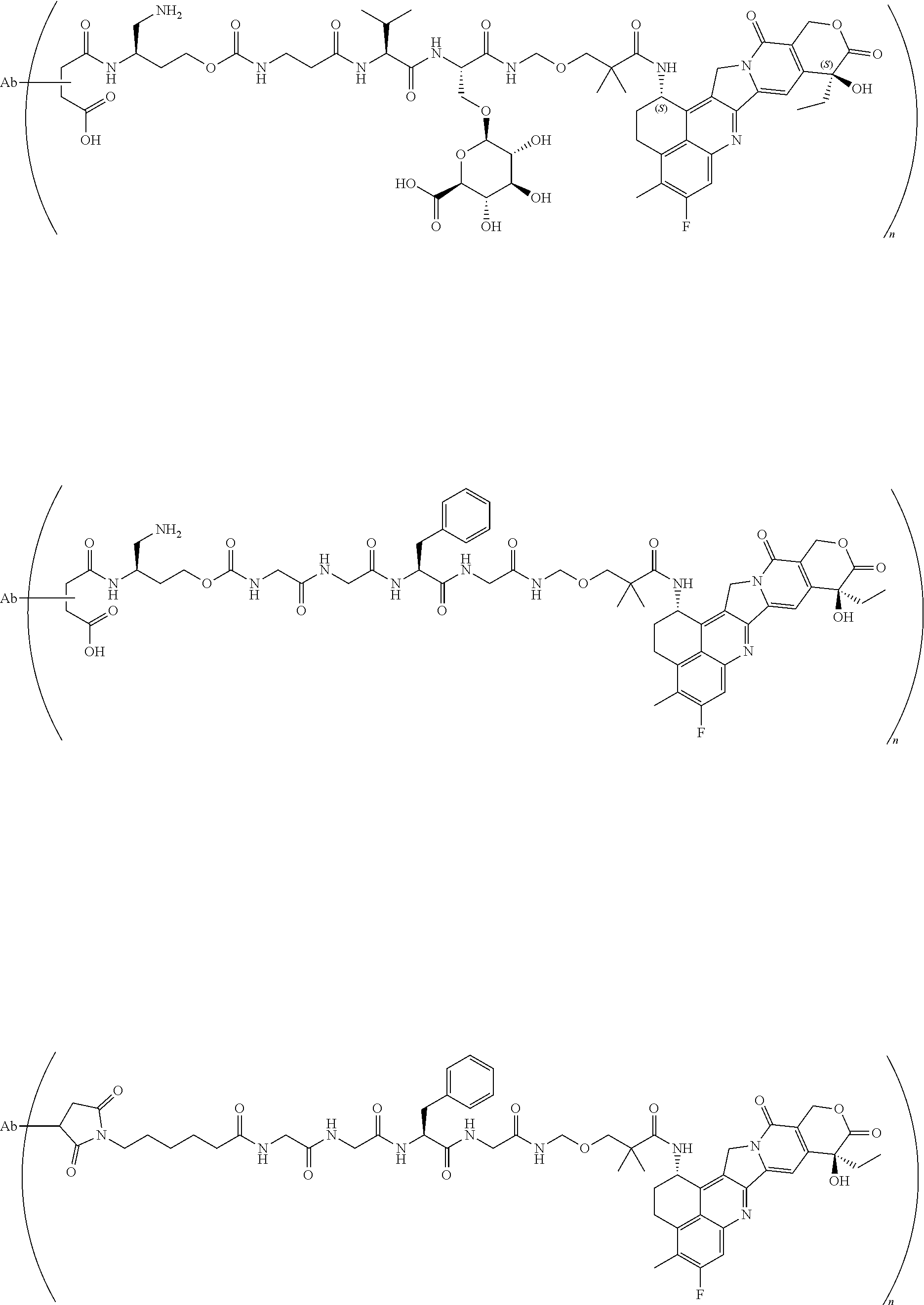

or a pharmaceutically acceptable salt, solvate, and/or stereoisomer thereof.

Embodiment 63. The antibody drug conjugate of any one of embodiments 1-62, wherein n is from 3 to 15, from 3 to 12, from 4 to 11, from 5 to 10, from 7 to 9, from 5 to 7, about 8, or about 6.

Embodiment 64. The antibody drug conjugate of embodiment 63, wherein n is about 8.

Embodiment 65. The antibody drug conjugate of embodiment 63, wherein n is about 6.

Embodiment 66. The antibody drug conjugate of any one of embodiments 1-44, wherein the antibody drug conjugate is represented by one of the following formulas:

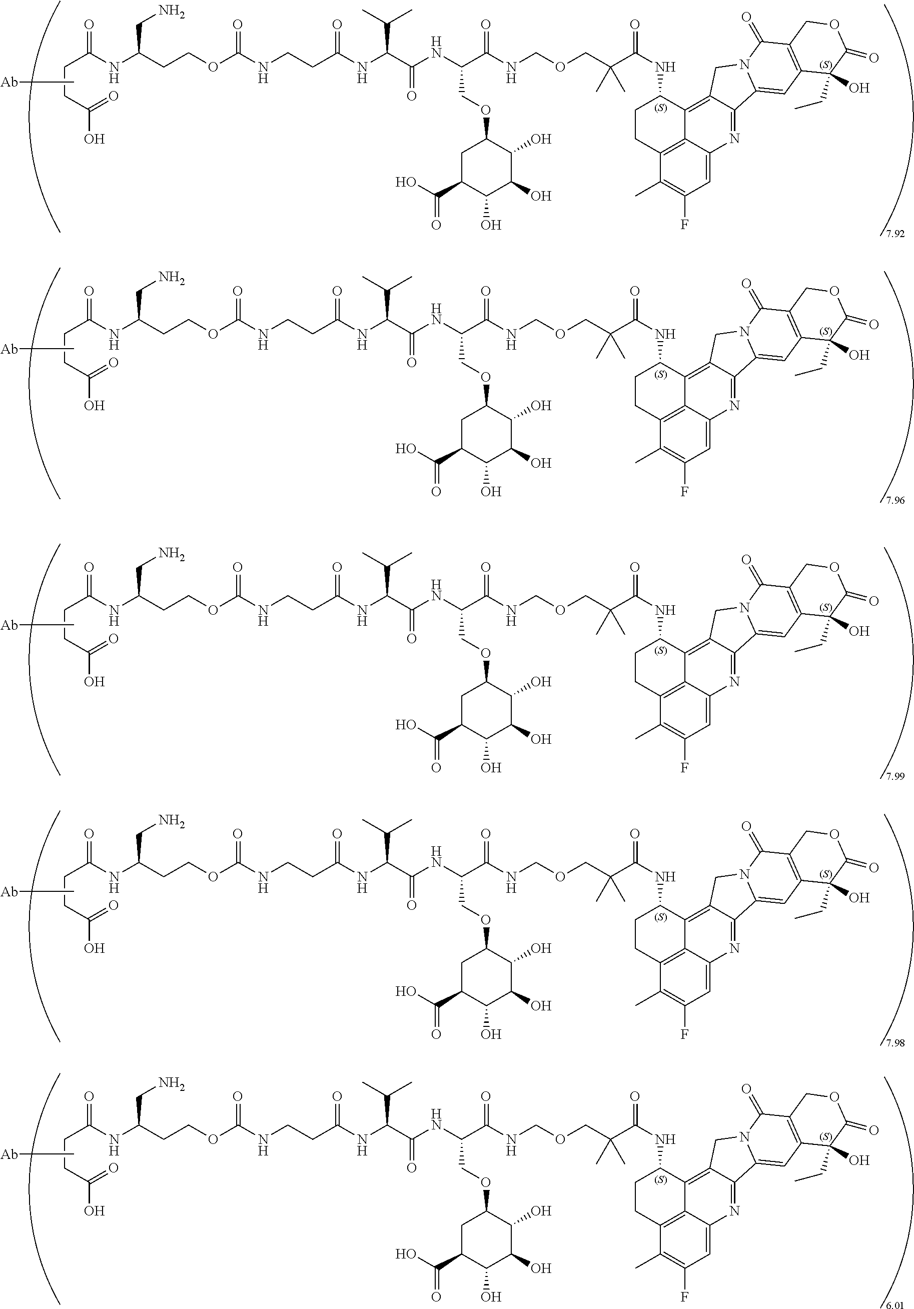

or a pharmaceutically acceptable salt, solvate, and/or stereoisomer thereof.

Embodiment 67. The antibody drug conjugate of any one of embodiments 1-44, wherein the antibody drug conjugate comprises one of the following formulas:

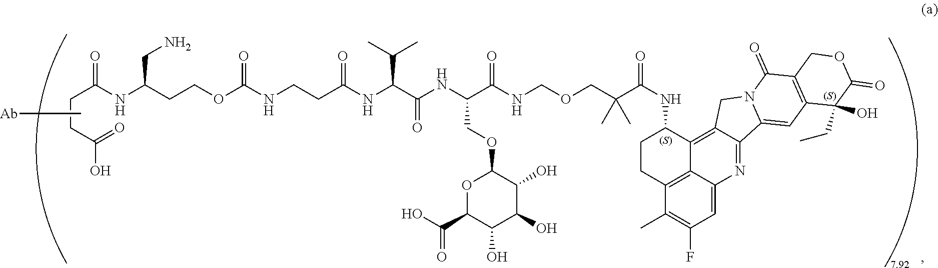

or a pharmaceutically acceptable salt, solvate, and/or stereoisomer thereof, wherein the Ab comprises a first polypeptide comprising the amino acid sequence of SEQ ID NO: 153, a second polypeptide comprising the amino acid sequence of SEQ ID NO: 170, and a third polypeptide comprising the amino acid sequence of SEQ ID NO: 148;

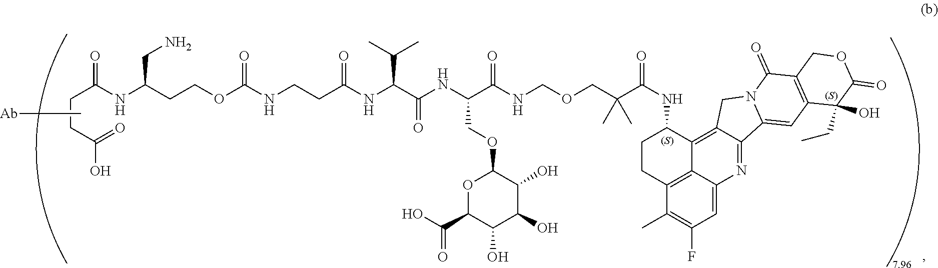

or a pharmaceutically acceptable salt, solvate, and/or stereoisomer thereof, wherein the Ab comprises a first polypeptide comprising the amino acid sequence of SEQ ID NO: 153, a second polypeptide comprising the amino acid sequence of SEQ ID NO: 172, and a third polypeptide comprising the amino acid sequence of SEQ ID NO: 148;

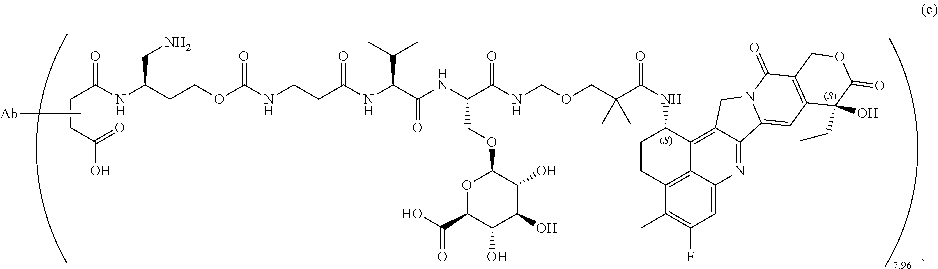

or a pharmaceutically acceptable salt, solvate, and/or stereoisomer thereof, wherein the Ab comprises a first polypeptide comprising the amino acid sequence of SEQ ID NO: 153, a second polypeptide comprising the amino acid sequence of SEQ ID NO: 150, and a third polypeptide comprising the amino acid sequence of SEQ ID NO: 148;

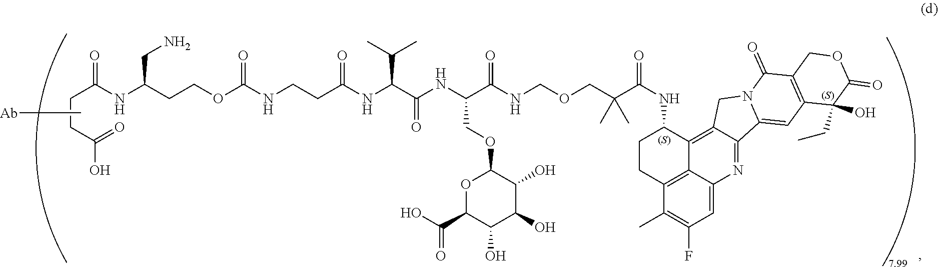

or a pharmaceutically acceptable salt, solvate, and/or stereoisomer thereof, wherein the Ab comprises a first polypeptide comprising the amino acid sequence of SEQ ID NO: 168, a second polypeptide comprising the amino acid sequence of SEQ ID NO: 169, and a third polypeptide comprising the amino acid sequence of SEQ ID NO: 148;

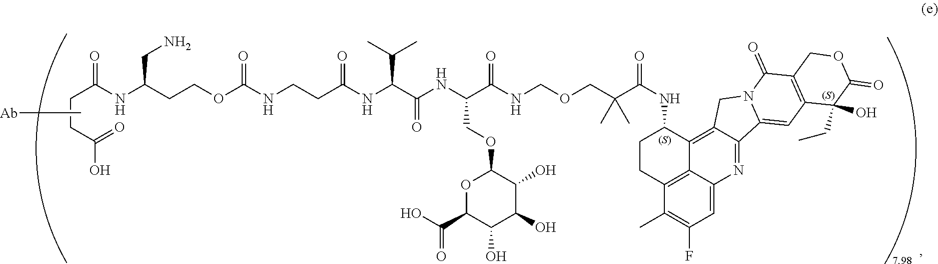

or a pharmaceutically acceptable salt, solvate, and/or stereoisomer thereof, wherein the Ab comprises a first polypeptide comprising the amino acid sequence of SEQ ID NO: 168, a second polypeptide comprising the amino acid sequence of SEQ ID NO: 171, and a third polypeptide comprising the amino acid sequence of SEQ ID NO: 148; or

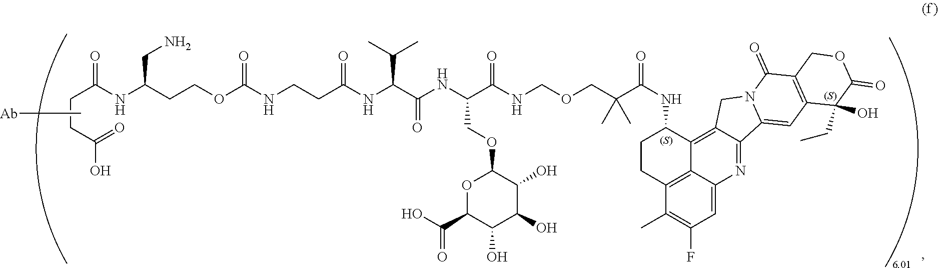

or a pharmaceutically acceptable salt, solvate, and/or stereoisomer thereof, wherein the Ab comprises a first polypeptide comprising the amino acid sequence of SEQ ID NO: 168, a second polypeptide comprising the amino acid sequence of SEQ ID NO: 171, and a third polypeptide comprising the amino acid sequence of SEQ ID NO: 148.

Embodiment 68. A pharmaceutical composition comprising the antibody drug conjugate of any one of embodiments 1-67 and a pharmaceutically acceptable carrier.

Embodiment 69. A method of treating a cancer comprising administering to a patient in need a therapeutically effective amount of the antibody drug conjugate of any one of embodiments 1-67, or the pharmaceutical composition of embodiment 68.

Embodiment 70. Use of the antibody drug conjugate of any one of embodiments 1-67, or the pharmaceutical composition of embodiment 68 in the manufacture of a medicament for treating cancer in a patient.

Embodiment 71. The antibody drug conjugate of any one of embodiments 1-67, or the pharmaceutical composition of embodiment 68 for use in treating cancer in a patient.

Embodiment 72. The method, use, or antibody drug conjugate of any one of embodiments 69-71, wherein the cancer harbors a cMET genetic alteration and/or the growth of the cancer cell is driven by cMET signaling.

Embodiment 73. The method, use, or antibody drug conjugate of embodiment 72, wherein the cMET signaling is ligand independent.

Embodiment 74. The method, use, or antibody drug conjugate of embodiment 72, wherein the cMET signaling is ligand dependent.

Embodiment 75. The method, use, or antibody drug conjugate of embodiment 72, wherein the cMET genetic alteration is cMET overexpression, genomic amplification, and/or mutation, which results in constitutively active cMET signaling.

Embodiment 76. The method, use, or antibody drug conjugate of any one of embodiments 69-75, wherein the cancer harbors an EGFR activating mutation and/or the growth of the cancer cell is driven by EGFR signaling; optionally, the EGFR activating mutation is deletion or point mutation.

Embodiment 77. The method, use, or antibody drug conjugate of embodiment 76, wherein the EGFR signaling is ligand independent.

Embodiment 78. The method, use, or antibody drug conjugate of embodiment 76, wherein the EGFR signaling is ligand dependent.

Embodiment 79. The method, use, or antibody drug conjugate of any one of embodiments 69-78, wherein the cancer is gastric cancer, colorectal cancer, or lung cancer.

Embodiment 80. The method, use, or antibody drug conjugate of embodiment 79, wherein the cancer is non-small cell lung cancer (NSCLC) or small cell lung cancer (SCLC).

Embodiment 81. A process for producing the antibody drug conjugate of any one of embodiments 1-67, comprising:
(i) culturing a host cell that has been transformed by an isolated nucleic acid comprising one or more sequences encoding the multispecific antibody or antigen-binding fragment thereof;
(ii) expressing the multispecific antibody or antigen-binding fragment thereof;
(iii) recovering the expressed multispecific antibody or antigen-binding fragment thereof; and
(iv) conjugating the payload (P) to the multispecific antibody or fragment thereof using the linker (L) and conjugator (C), such that the antibody drug conjugate is formed.

This Summary is neither intended as, nor should it be construed as, being representative of the full extent and scope of the present disclosure. Moreover, references made herein to “the present disclosure,” or aspects thereof, should be understood to mean certain embodiments of the present disclosure and should not be construed as limiting all embodiments to a particular description. The present disclosure is set forth in various levels of detail in this Summary as well as in the Detailed Description and accompanying drawings, and no limitation as to the scope of the present disclosure is intended by either the inclusion or non-inclusion of elements, components, etc. in this Summary. Features from any of the disclosed embodiments may be used in combination with one another, without limitation. In addition, other features and advantages of the present disclosure will become apparent to those of ordinary skill in the art through consideration of the following Detailed Description and the accompanying drawings.

### BRIEF DESCRIPTION OF THE DRAWINGS

FIGS. 1A-1L show the SPR binding kinetics curve of representative monoclonal anti-cMET antibodies. FIG. 1A: 063Ab10910; FIG. 1B: 061Ab15310; FIG. 1C: 063Ab16010; FIG. 1D: 063Ab02110; FIG. 1E: 063Ab15210; FIG. 1F: 062Ab16310; FIG. 1G: 063Ab05510; FIG. 1H: 063Ab07710; FIG. 1I: 063Ab14710; FIG. 1J: 061Ab05110; FIG. 1K: 063Ab03210; FIG. 1L: 063Ab16720.

FIG. 2 shows the comparison of ligand-induced signaling blocking activity of representative monoclonal anti-cMET antibodies.

FIG. 3 shows the epitope binning results of representative monoclonal anti-cMET antibodies.

FIG. 4 shows the inhibition of ligand-independent signaling by bispecific anti-cMET antibodies in Hs746T cells.

FIG. 5 shows the anti-proliferation activity of bispecific anti-cMET antibodies against Hs746T cells.

FIG. 6 shows the ligand-induced signaling blocking of bispecific anti-cMET antibodies in H596 cells.

FIG. 7 shows different EGFR×cMET×cMET trispecific antibody formats, with different EGFR scFv valency and orientation to cMET arms.

FIGS. 8A-8D show ADCC activity of EGFR×cMET×cMET trispecific antibodies on H1975 cells without (FIG. 8A) and with serum (FIG. 8B) and Hs746T cells without (FIG. 8C) and with serum (FIG. 8D).

FIGS. 9A-9D show ADCP activity of EGFR×cMET×cMET trispecific antibodies on H1975 cells without (FIG. 9A) and with serum (FIG. 9B) and Hs746T without (FIG. 9C) and with serum (FIG. 9D).

FIGS. 10A-10D show anti-proliferation activity of EGFR×cMET×cMET trispecific antibodies on EGFR signaling-dependent cancer cell line H1975 (FIG. 10A) and H2073 (FIG. 10B) and cMET signaling-dependent cancer cell line Hs746T (FIG. 10C) and EBC-1 (FIG. 10D).

FIG. 11 shows anti-proliferation activity of EGFR×cMET×cMET trispecific antibodies against HEKn cells.

FIG. 12 shows EGFR×cMET×cMET trispecific antibody TE-642 showed lower TGI and CR rate in HCC827 xenograft model (EGFR mutation driven model), compared with JNJ-372.

FIG. 13 shows EGFR×cMET×cMET trispecific antibody TE-647 showed comparable tumor growth inhibition to JNJ-372 in HCC827 xenograft model (EGFR mutation driven model).

FIG. 14 shows EGFR×cMET×cMET trispecific antibodies TE-642 and TE-646 showed tumor growth inhibition in H1975 xenograft model (EGFR mutation driven model).

FIG. 15 shows EGFR×cMET×cMET trispecific antibody TE-647 showed comparable tumor growth inhibition to JNJ-372 in H1975 xenograft model (EGFR mutation driven model).

FIG. 16 shows EGFR×cMET×cMET trispecific antibody TE-647 showed better tumor growth inhibition than JNJ-372 in Hs746T xenograft model (cMET amplification driven model), and the effect is dose dependent.

FIG. 17 shows EGFR×cMET×cMET antibody TE-647 showed better in vivo activity than JNJ-372 in EBC-1 (cMET amplification driven model) xenograft model, and the effect is dose dependent.

FIG. 18 is a schematic of an anti-EGFR×cMET×cMET trispecific antibody according to one embodiment.

FIGS. 19A and 19B show ADC internalization into HCC827 (FIG. 19A) or H1975 (FIG. 19B) cells.

FIGS. 20A-23B show direct killing by ADCs of HCC827 (FIGS. 20A-20C), H441 (FIGS. 21A-21C), HT-29 (FIGS. 22A-22C), and H520 (FIGS. 23A-23B) cells.

FIGS. 24A-24B shows ADC bystander killing effects in co-cultured HCC827 and H520-nanoLuc cells.

FIGS. 25-33 show in vitro ADC efficacy in human colon (FIGS. 25, 26, and 30) and lung (FIGS. 27-29 and 31-33) cancer xenograft models.

### DEFINITIONS

Unless specifically defined below or elsewhere in this document, all technical and scientific terms used herein have the meaning commonly understood by one of ordinary skill in the art.

As used herein, including in the appended claims, the singular forms of words such as “a,” “an,” and “the” include their corresponding plural forms unless the context clearly indicates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “or” is used to mean, and is used interchangeably with, the term “and/or” unless the context clearly indicates otherwise.

As used herein, including in the claims that follow, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

The term “cMET” refers to receptor tyrosine kinase mesenchymal-epithelial transition factor. The amino acid sequence of human cMET (SEQ ID NO: 40) can also be found in GenBank: AAI30421.1.

The term “EGFR” refers to epidermal growth factor receptor. The amino acid sequence of human EGFR (SEQ ID NO: 41) can be found at https://www.uniprot.org/uniprotkb/P00533/entry#sequences.

The term “cMET biparatopic antibodies or antigen-binding fragments thereof” means in the multispecific antibodies or antigen-binding fragments thereof, the first antigen binding domain thereof targeting human cMET and the second antigen binding domain thereof targeting human cMET recognize non-overlapping epitopes on human cMET, or the first antigen binding domain thereof targeting human cMET does not compete with the second antigen binding domain thereof targeting human cMET.

The terms “administration,” and “administering,” as used herein, when applied to an animal, human, subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The terms “improve,” “increase,” “inhibit,” and “reduce” indicate values that are relative to a baseline or other reference measurement. In some embodiments, an appropriate reference measurement may comprise a measurement in a certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in a comparable system known or expected to respond in a comparable way, in presence of the relevant agent or treatment.

The terms “cancer” or “tumor” used herein has the broadest meaning as understood in the art and refers to the physiological condition in mammals that is typically characterized by unregulated cell growth. In the context of the present disclosure, the cancer or tumor is not limited to a certain type or location.

As used herein, the term “amino acid” refers to an organic compound that contains amino (—NH₂) and carboxyl (—COOH) functional groups, along with a side chain (R group), which is specific to each amino acid. Amino acids, as used herein, may be natural or unnatural, synthetic, proteinogenic or non-proteinogenic, D or L optical isomers, analogs, and/or peptidomimetics. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide,” in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“Aho” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL or Vx) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA, and IgY), or subclass (e.g., IgG1, IgG2, IgG3, IgG4, IgAQ1, and IgA2).

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies can be obtained by methods known to those skilled in the art. See, for example, Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: a LABORATORY MANUAL, Cold Spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies and antibodies formed from antibody fragments; and bicyclic peptides (Hurov, K. et al., 2021. Journal for ImmunoTherapy of Cancer, 9(11)).

As used herein, an antibody or antigen-binding antibody fragment, “specifically binds” or “selectively binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a blood, serum, plasma, or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof specifically bind to a particular antigen at least ten times greater when compared to the background level of binding and does not specifically bind in a significant amount to other antigens present in the sample.

The term “affinity” as used herein refers to the strength of interaction between antibody and antigen. Within the antigen, the variable regions of the antibody interact through non-covalent forces with the antigen at numerous sites. In general, the more interactions, the stronger the affinity.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification, or for other reasons.

The term “equilibrium dissociation constant” or “K_{D}” or “M” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M, or 10⁻¹³ M.

In the context of the present disclosure, when reference is made to an amino acid sequence, the term “conservative substitution” means substitution of the original amino acid by a new amino acid that does not substantially alter the chemical, physical, and/or functional properties of the antibody or fragment, e.g., its binding affinity to cMET and/or EGFR. Common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces, or V_{H}/V_{L} interfaces of antibodies (see, e.g., US 2011/0287009, US2007/0178552, WO 96/027011, WO 98/050431, and Zhu et al., 1997, Protein Science 6:781-788). In some embodiments, knob-into-holes ensure the correct pairing of two different heavy chains together during the manufacture of antibodies. For example, antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also ensure correct pairing.

Examples of algorithms that are suitable for determining percent sequence identity and sequence similarity are the BLAST algorithms, which are described in Altschul et al., Nuc. Acids Res. 25:3389-3402, 1977; and Altschul et al., J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915), alignments (B) of 50, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12, and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include antibodies or antigen-binding fragments thereof as disclosed herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal, or epidermal administration (e.g., by injection or infusion).

The term “therapeutically effective amount” or “effective amount” as herein used refers to the amount of an agent that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “combination therapy” refers to the administration of two or more therapeutic agents to treat a therapeutic condition or disorder. Such administration encompasses co-administration of these therapeutic agents in a substantially simultaneous manner. Such administration also encompasses co-administration in multiple or in separate containers or formulations (e.g., capsules, powders, and liquids) for each active ingredient. Powders and/or liquids can be reconstituted or diluted to a desired dose prior to administration. In addition, “combination therapy” encompasses use of each type of therapeutic agent in a sequential manner, either at approximately the same time or at different times. In either case, the treatment regimen will provide beneficial effects of the drug combination in treating the conditions or disorders described herein.

The term “toxin” or “payload” or “cytotoxic agent” is used herein to reference a molecule that inhibits or reduces the expression of molecules in cells, inhibits or reduces the function of cells, induces apoptosis of cells, and/or causes death of cells. The term includes radioactive isotopes, chemotherapeutic agents, and toxins such as small molecule toxins or enzymatically active toxins of bacterial, fungal, plant, or animal origin, including fragments and/or variants thereof. Examples of cytotoxic agents include, but are not limited to, auristatins (e.g., auristatin E, auristatin F, MMAE, and MMAF), auromycins, maytansinoids, pyrrolobenzodiazepine (PBD), ricin, ricin A-chain, combrestatin, duocarmycins, dolastatins, doxorubicin, daunorubicin, taxols, cisplatin, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, and calicheamicin, as well as radioisotopes such as At211, I131, I125, Y90, Re186, Re188, Sm153, Bi212 or 213, P32, and Lu177.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl, and n-hexyl; saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

An “arylene” group is a bivalent aryl group as defined herein.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heteroarylene” group is a bivalent heteroaryl group as defined herein.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amino” group is a radical of the formula NH₂.

A “hydroxyl amine” group is a radical of the formula N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R^{#} is as defined above.

An “aralkoxyamine” group is a radical of the formula N(R^{#})O-aryl or NHOaryl, wherein R^{#} is as defined above.

An “alkylamine” group is a radical of the formula NHalkyl or N(alkyl)₂, wherein each alkyl is independently as defined above.

An “aminocarbonyl” group is a radical of the formula —C(═O)N(R^{#})₂, —C(═O)NH(R^{#}), or C(═O)NH₂, wherein each R^{#} is as defined above.

An “acylamino” group is a radical of the formula NHC(═O)(R^{#}) or N(alkyl)C(═O)(R^{#}), wherein each alkyl and R^{#} are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O⁻.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂#, wherein each alkyl and R^{#} are independently as defined above.

An “imine” group is a radical of the formula —N═C(R^{#})₂ or —C(R^{#})═N(R^{#}), wherein each R^{#} is independently as defined above.

An “imide” group is a radical of the formula —C(═O)N(R^{#})C(═O)(R^{#}) or N((C═O)(R^{#}))₂, wherein each R^{#} is independently as defined above.

A “urethane” group is a radical of the formula —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R^{#}), wherein R^{#} is as defined above.

A “sulfinyl” group is a radical of the formula —S(═O)(R^{#}), wherein R^{#} is as defined above.

A “sulfone” group is a radical of the formula —S(═O)₂(R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, or —S(═O)₂NH(R^{#}), or —S(═O)₂NH₂, wherein each R^{#} is independently as defined above.

A “phosphonate” group is a radical of the formula —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula —P(R^{#})₂, wherein each R^{#} is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

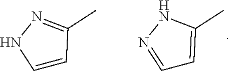

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²¹I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “inorganic acid residue” refers to an ortho- or pyrophosphoric acid or sulphuric acid residue.

As used herein, “organic acid residue” refers to the residue of alkanecarboxylic acid, an amino acid, or an oligopeptide. In embodiments, the alkanecarboxylic acid is methanoic acid, ethanoic acid, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, or icosanoic acid. In embodiments, the alkanecarboxylic acid is methanoic acid, ethanoic acid, propanoic acid, or butanoic acid.

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

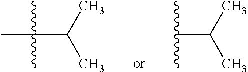

has the following structure:

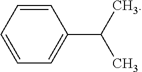

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains.

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

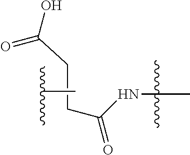

encompasses

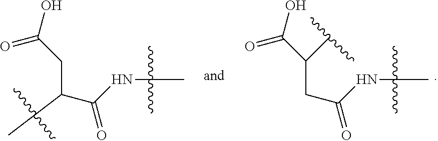 [SMILES: *NC(=O)C(*)CC(=O)O ; *NC(=O)CC(*)C(=O)O]

As used herein, the term “cell-killing activity” refers to activity that decreases or reduces the cell viability of a tested cell line.

### DETAILED DESCRIPTION

The present disclosure provides antibody drug conjugates (ADC) of multispecific antibodies or antibody fragments thereof that specifically bind to human EGFR and two distinct epitopes of human cMET (e.g., non-overlapping) or multispecific antibodies or antigen-binding fragments thereof that specifically bind to human EGFR and human cMET, wherein the first antigen binding domain thereof targeting human cMET does not compete with the second antigen binding domain thereof targeting human cMET. Also, the present disclosure provides multispecific antibodies or antibody fragments thereof that have desirable binding affinity, desirable ADCC activity, desirable ADCP activity, desirable blocking activity on EGFR signaling and cMET signaling, desirable anti-proliferation activity and killing effect on EGFR signaling driven cancer cells and cMET signaling driven cancer cells. In some embodiments, the ADCs of the multispecific antibodies and the fragments thereof could be used for reducing the likelihood of or treating cancer and associated disorders. The present disclosure also includes methods of preparing the antibody drug conjugates herein.

### I. Anti-cMET Antigen Binding Domains

Antibody drug conjugates of the present disclosure include, for example, a first antigen binding domain that specifically binds to a first epitope of human cMET. Such first antigen binding domains include, but are not limited to, antigen binding domains as described below.

### 1.1 First Group of Anti-cMET Antigen Binding Domains

TABLE 1
Anti-cMET Monoclonal Antibody List 1

| | SEQ ID NO. | | | | | | | | | |
|---|---|---|---|---|---|---|---|---|---|---|
| | VH | VH | HCDR1 | HCDR2 | HCDR3 | VL | VL | LCDR1 | LCDR2 | LCDR3 |
| Antibody | (AA) | (DNA) | (Kabat) | (Kabat) | (Kabat) | (AA) | (DNA) | (Kabat) | (Kabat) | (Kabat) |
| 063Ab10910 | 4 | 5 | 6 | 7 | 8 | 64 | 65 | 66 | 67 | 68 |
| 063Ab10910- | 72 | / | 6 | 7 | 8 | 64 | 65 | 66 | 67 | 68 |
| V1 | | | | | | | | | | |
| 063Ab10910 | / | / | 6 | 7 | 94 | 64 | 65 | 66 | 67 | 68 |
| variants | | | | | (formula) | | | | | |
| 063Ab10910- | 85 | / | 6 | 7 | 84 | 64 | 65 | 66 | 67 | 68 |
| P28 | | | | | | | | | | |
| 063Ab10910- | 87 | / | 6 | 7 | 86 | 64 | 65 | 66 | 67 | 68 |
| P27 | | | | | | | | | | |
| 063Ab10910- | 89 | / | 6 | 7 | 88 | 64 | 65 | 66 | 67 | 68 |
| P37 | | | | | | | | | | |
| 063Ab10910- | 91 | / | 6 | 7 | 90 | 64 | 65 | 66 | 67 | 68 |
| P26 | | | | | | | | | | |
| 063Ab10910- | 93 | / | 6 | 7 | 92 | 64 | 65 | 66 | 67 | 68 |
| P19 | | | | | | | | | | |
| BGA-109 | 144 | 162 | 6 | 7 | 92 | 64 | 65 | 66 | 67 | 68 |
| 061Ab15310 | 9 | 10 | 11 | 12 | 13 | 64 | 65 | 66 | 67 | 68 |
| 061Ab15310- | 73 | / | 11 | 12 | 13 | 64 | 65 | 66 | 67 | 68 |
| V1 | | | | | | | | | | |
| 063Ab16010 | 14 | 15 | 16 | 17 | 18 | 64 | 65 | 66 | 67 | 68 |
| 063Ab16010- | 74 | / | 16 | 17 | 18 | 64 | 65 | 66 | 67 | 68 |
| V1 | | | | | | | | | | |
| 063Ab02110 | 19 | 20 | 21 | 22 | 23 | 64 | 65 | 66 | 67 | 68 |
| 063Ab02110- | 75 | / | 21 | 22 | 23 | 64 | 65 | 66 | 67 | 68 |
| V1 | | | | | | | | | | |
| 063Ab15210 | 24 | 25 | 26 | 27 | 28 | 64 | 65 | 66 | 67 | 68 |
| 063Ab15210- | 76 | / | 26 | 27 | 28 | 64 | 65 | 66 | 67 | 68 |
| V1 | | | | | | | | | | |
| 062Ab16310 | 29 | 30 | 31 | 32 | 33 | 64 | 65 | 66 | 67 | 68 |
| 062Ab16310- | 77 | / | 31 | 32 | 33 | 64 | 65 | 66 | 67 | 68 |
| V1 | | | | | | | | | | |
| 063Ab05510 | 34 | 35 | 36 | 37 | 38 | 64 | 65 | 66 | 67 | 68 |
| 063Ab05510- | 78 | / | 36 | 37 | 38 | 64 | 65 | 66 | 67 | 68 |
| V1 | | | | | | | | | | |
| 063Ab07710 | 39 | 40 | 41 | 42 | 43 | 64 | 65 | 66 | 67 | 68 |
| 063Ab07710- | 79 | / | 41 | 42 | 43 | 64 | 65 | 66 | 67 | 68 |
| V1 | | | | | | | | | | |
| 063Ab14710 | 44 | 45 | 46 | 47 | 48 | 64 | 65 | 66 | 67 | 68 |
| 063Ab14710- | 80 | / | 46 | 47 | 48 | 64 | 65 | 66 | 67 | 68 |
| V1 | | | | | | | | | | |
| 061Ab05110 | 49 | 50 | 51 | 52 | 53 | 64 | 65 | 66 | 67 | 68 |
| 061Ab05110- | 81 | / | 51 | 52 | 53 | 64 | 65 | 66 | 67 | 68 |
| V1 | | | | | | | | | | |

In some embodiments, the first antigen binding domain is derived from an anti-cMET antibody or antigen-binding fragment thereof comprising heavy chain complementarity determining regions (HCDRs), light chain CDRs (LCDRs), variable heavy chain (VH), and/or variable light chain (VL) sequences comprising the SEQ ID NOS described in Table 1 above. In some embodiments, the first antigen binding domain comprises a VH domain comprising an amino acid sequence of SEQ ID NO: 4, SEQ ID NO: 72, SEQ ID NO: 85, SEQ ID NO: 87, SEQ ID NO: 89, SEQ ID NO: 91, SEQ ID NO: 93, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81 or SEQ ID NO: 144 (Table 1). In some embodiments, the first antigen binding domain comprises a HCDR comprising an amino acid sequence of any one of the HCDRs listed in Table 1. In some embodiments, the first antigen binding domain comprises one, two, or three, HCDRs comprising an amino acid sequence of any of the antibodies listed in Table 1.

In some embodiments, the first antigen binding domain comprises a VL domain comprising an amino acid sequence of SEQ ID NO: 64 (Table 1). In some embodiments, the first antigen binding domain comprises a LCDR comprising an amino acid sequence of any one of the LCDRs listed in Table 1. In some embodiments, the first antigen binding domain comprises one, two, or three, LCDRs comprising an amino acid sequence of any of the LCDRs listed in Table 1.

In some embodiments, the first antigen binding domain comprises one or more complementarity determining regions (CDRs) comprising an amino acid sequence selected from a group consisting of SEQ ID NO: 6, SEQ ID NO: 7, SEQ ID NO: 8, SEQ ID NO: 94, SEQ ID NO: 84; SEQ ID NO: 86, SEQ ID NO: 88, SEQ ID NO: 90, SEQ ID NO: 92, SEQ ID NO: 11, SEQ ID NO: 12, SEQ ID NO: 13, SEQ ID NO: 16, SEQ ID NO: 17, SEQ ID NO: 18, SEQ ID NO: 21, SEQ ID NO: 22, SEQ ID NO: 23, SEQ ID NO: 26, SEQ ID NO: 27, SEQ ID NO: 28, SEQ ID NO: 31, SEQ ID NO: 32, SEQ ID NO: 33, SEQ ID NO: 36, SEQ ID NO: 37, SEQ ID NO: 38, SEQ ID NO: 41, SEQ ID NO: 42, SEQ ID NO: 43, SEQ ID NO: 46, SEQ ID NO: 47, SEQ ID NO: 48, SEQ ID NO: 51, SEQ ID NO: 52, SEQ ID NO: 53, SEQ ID NO: 66, SEQ ID NO: 67 and SEQ ID NO: 68.

In some embodiments, the first antigen binding domain comprises: (a) a heavy chain variable region comprising one or more complementarity determining regions (HCDRs) comprising an amino acid sequence selected from the group consisting of SEQ ID NO: 6, SEQ ID NO: 7, SEQ ID NO: 8, SEQ ID NO: 94, SEQ ID NO: 84; SEQ ID NO: 86, SEQ ID NO: 88, SEQ ID NO: 90, SEQ ID NO: 92, SEQ ID NO: 11, SEQ ID NO: 12, SEQ ID NO: 13, SEQ ID NO: 16, SEQ ID NO: 17, SEQ ID NO: 18, SEQ ID NO: 21, SEQ ID NO: 22, SEQ ID NO: 23, SEQ ID NO: 26, SEQ ID NO: 27, SEQ ID NO: 28, SEQ ID NO: 31, SEQ ID NO: 32, SEQ ID NO: 33, SEQ ID NO: 36, SEQ ID NO: 37, SEQ ID NO: 38, SEQ ID NO: 41, SEQ ID NO: 42, SEQ ID NO: 43, SEQ ID NO: 46, SEQ ID NO: 47, SEQ ID NO: 48, SEQ ID NO: 51, SEQ ID NO: 52, and SEQ ID NO: 53, and/or (b) a light chain variable region comprising one or more complementarity determining regions (LCDRs) comprising an amino acid sequence selected from the group consisting of SEQ ID NO: 66, SEQ ID NO: 67 and SEQ ID NO: 68.

In some embodiments, the first antigen binding domain comprises: (a) a heavy chain variable region comprising three complementarity determining regions (HCDRs) which are HCDR1 comprising an amino acid sequence of SEQ ID NO: 6, SEQ ID NO: 11, SEQ ID NO: 16, SEQ ID NO: 21, SEQ ID NO: 26, SEQ ID NO: 31, SEQ ID NO: 36, SEQ ID NO: 41, SEQ ID NO: 46, or SEQ ID NO: 51; HCDR2 comprising an amino acid sequence of SEQ ID NO: 7, SEQ ID NO: 12, SEQ ID NO: 17, SEQ ID NO: 22, SEQ ID NO: 27, SEQ ID NO: 32, SEQ ID NO: 37, SEQ ID NO: 42, SEQ ID NO: 47, or SEQ ID NO: 52; and HCDR3 comprising an amino acid sequence of SEQ ID NO: 8, SEQ ID NO: 94, SEQ ID NO: 84; SEQ ID NO: 86, SEQ ID NO: 88, SEQ ID NO: 90, SEQ ID NO: 92, SEQ ID NO: 13, SEQ ID NO: 18, SEQ ID NO: 23, SEQ ID NO: 28, SEQ ID NO: 33, SEQ ID NO: 38, SEQ ID NO: 43, SEQ ID NO: 48, or SEQ ID NO: 53; and/or (b) a light chain variable region comprising three complementarity determining regions (LCDRs) which are LCDR1 comprising an amino acid sequence of SEQ ID NO: 66; LCDR2 comprising an amino acid sequence of SEQ ID NO: 67; and LCDR3 comprising an amino acid sequence of SEQ ID NO: 68.

In some embodiments, the first antigen binding domain comprises: (a) a heavy chain variable region comprising three complementarity determining regions (HCDRs) which are: HCDR1 comprising an amino acid sequence of SEQ ID NO: 6, HCDR2 comprising an amino acid sequence of SEQ ID NO: 7, and HCDR3 comprising an amino acid sequence of SEQ ID NO: 8; HCDR1 comprising an amino acid sequence of SEQ ID NO: 6, HCDR2 comprising an amino acid sequence of SEQ ID NO: 7, and HCDR3 comprising an amino acid sequence of SEQ ID NO: 94; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 84; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 86; HCDR1 comprising an amino acid sequence of SEQ ID NO: 6, HCDR2 comprising an amino acid sequence of SEQ ID NO: 7, and HCDR3 comprising an amino acid sequence of SEQ ID NO: 88; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 90; HCDR1 comprising an amino acid sequence of SEQ ID NO: SEQ ID NO: 6, HCDR2 comprising an amino acid sequence of SEQ ID NO: 7, and HCDR3 comprising an amino acid sequence of SEQ ID NO: 92; HCDR1 comprising an amino acid sequence of SEQ ID NO: SEQ ID NO: 11, HCDR2 comprising an amino acid sequence of SEQ ID NO: 12, and HCDR3 comprising an amino acid sequence of SEQ ID NO: 13; HCDR1 comprising an amino acid sequence of SEQ ID NO: SEQ ID NO: 16, HCDR2 comprising an amino acid sequence of SEQ ID NO: 17, and HCDR3 comprising an amino acid sequence of SEQ ID NO: 18; HCDR1 comprising an amino acid sequence of SEQ ID NO: SEQ ID NO: 21, HCDR2 comprising an amino acid sequence of SEQ ID NO: 22, and HCDR3 comprising an amino acid sequence of SEQ ID NO: 23; HCDR1 comprising an amino acid sequence of SEQ ID NO: SEQ ID NO: 26, HCDR2 comprising an amino acid sequence of SEQ ID NO: 27, and HCDR3 comprising an amino acid sequence of SEQ ID NO: 28; HCDR1 comprising an amino acid sequence of SEQ ID NO: SEQ ID NO: 31, HCDR2 comprising an amino acid sequence of SEQ ID NO: 32, and HCDR3 comprising an amino acid sequence of SEQ ID NO: 33; HCDR1 comprising an amino acid sequence of SEQ ID NO: SEQ ID NO: 36, HCDR2 comprising an amino acid sequence of SEQ ID NO: 37, and HCDR3 comprising an amino acid sequence of SEQ ID NO: 38; HCDR1 comprising an amino acid sequence of SEQ ID NO: SEQ ID NO: 41, HCDR2 comprising an amino acid sequence of SEQ ID NO: 42, and HCDR3 comprising an amino acid sequence of SEQ ID NO: 43; HCDR1 comprising an amino acid sequence of SEQ ID NO: SEQ ID NO: 46, HCDR2 comprising an amino acid sequence of SEQ ID NO: 47, and HCDR3 comprising an amino acid sequence of SEQ ID NO: 48; or HCDR1 comprising an amino acid sequence of SEQ ID NO: SEQ ID NO: 51, HCDR2 comprising an amino acid sequence of SEQ ID NO: 52, and HCDR3 comprising an amino acid sequence of SEQ ID NO: 53, and/or (b) a light chain variable region comprising three complementarity determining regions (LCDRs) which are LCDR1 comprising an amino acid sequence of SEQ ID NO: 66, LCDR2 comprising an amino acid sequence of SEQ ID NO: 67, and LCDR3 comprising an amino acid sequence of SEQ ID NO: 68.

In some embodiments, the first antigen binding domain comprises: (1) a HCDR1 (Heavy Chain Complementarity Determining Region 1), a HCDR2 and a HCDR3 from the heavy chain variable region (VH) set forth in SEQ ID NO: 4 or SEQ ID NO: 72; (2) a HCDR1, a HCDR2 and a HCDR3 from the heavy chain variable region (VH) set forth in SEQ ID NO: 85; (3) a HCDR1, a HCDR2 and a HCDR3 from the heavy chain variable region (VH) set forth in SEQ ID NO: 87; (4) a HCDR1, a HCDR2 and a HCDR3 from the heavy chain variable region (VH) set forth in SEQ ID NO: 89; (5) a HCDR1, a HCDR2 and a HCDR3 from the heavy chain variable region (VH) set forth in SEQ ID NO: 91; (6) a HCDR1, a HCDR2 and a HCDR3 from the heavy chain variable region (VH) set forth in SEQ ID NO: 93; (7) a HCDR1, a HCDR2 and a HCDR3 from the heavy chain variable region (VH) set forth in SEQ ID NO: 9 or SEQ ID NO: 73; (8) a HCDR1, a HCDR2 and a HCDR3 from the heavy chain variable region (VH) set forth in SEQ ID NO: 14 or SEQ ID NO: 74; (9) a HCDR1, a HCDR2 and a HCDR3 from the heavy chain variable region (VH) set forth in SEQ ID NO: 19 or SEQ ID NO: 75; (10) a HCDR1, a HCDR2 and a HCDR3 from the heavy chain variable region (VH) set forth in SEQ ID NO: 24 or SEQ ID NO: 76; (11) a HCDR1, a HCDR2 and a HCDR3 from the heavy chain variable region (VH) set forth in SEQ ID NO: 29 or SEQ ID NO: 77; (12) a HCDR1, a HCDR2 and a HCDR3 from the heavy chain variable region (VH) set forth in SEQ ID NO: 34 or SEQ ID NO: 78; (13) a HCDR1, a HCDR2 and a HCDR3 from the heavy chain variable region (VH) set forth in SEQ ID NO: 39 or SEQ ID NO: 79; (14) a HCDR1, a HCDR2 and a HCDR3 from the heavy chain variable region (VH) set forth in SEQ ID NO: 44 or SEQ ID NO: 80; (15) a HCDR1, a HCDR2 and a HCDR3 from the heavy chain variable region (VH) set forth in SEQ ID NO: 49 or SEQ ID NO: 81; or (16) a HCDR1, a HCDR2 and a HCDR3 from the heavy chain variable region (VH) set forth in SEQ ID NO: 144; and/or (1) a LCDR1 (Light Chain Complementarity Determining Region 1), a LCDR2 and a LCDR3 from the light chain variable region (VL) set forth in SEQ ID NO: 64.

In some embodiments, the first antigen binding domain comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(3) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(4) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(5) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(6) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68.
(7) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(11) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(12) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(13) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(14) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(15) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68; or
(16) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68, according to the Kabat definition.

In some embodiments, the first antigen binding domain comprises: (a) a heavy chain variable region comprising an amino acid sequence of SEQ ID NO: 4, SEQ ID NO: 72, SEQ ID NO: 85, SEQ ID NO: 87, SEQ ID NO: 89, SEQ ID NO: 91, SEQ ID NO: 93, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81 or SEQ ID NO: 144; or an amino acid sequence at least 90%, 91%, 92%, 93%, 94%, 95%, 96%, 97%, 98% or 99% identical to any one of SEQ ID NO: 4, SEQ ID NO: 72, SEQ ID NO: 85, SEQ ID NO: 87, SEQ ID NO: 89, SEQ ID NO: 91, SEQ ID NO: 93, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81 or SEQ ID NO: 144 and/or (b) a light chain variable region comprising an amino acid sequence of SEQ ID NO: 64, or an amino acid sequence at least 90%, 91%, 92%, 93%, 94%, 95%, 96%, 97%, 98% or 99% identical to SEQ ID NO: 64.

In some embodiments, the first antigen binding domain comprises:
(1) a heavy chain variable region (VH) comprising an amino acid sequence at least 90, 91, 92, 93, 94, 95, 96, 97, 98 or 99% identical to SEQ ID NO: 85, SEQ ID NO: 87, SEQ ID NO: 89, SEQ ID NO: 91, or SEQ ID NO: 93, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 4 or SEQ ID NO: 72, and a light chain variable region (VL) comprising an amino acid sequence at least 90, 91, 92, 93, 94, 95, 96, 97, 98 or 99% identical to SEQ ID NO: 64;
(3) a heavy chain variable region (VH) comprising an amino acid sequence at least 90, 91, 92, 93, 94, 95, 96, 97, 98 or 99% identical to SEQ ID NO: 9 or SEQ ID NO: 73, and a light chain variable region (VL) comprising an amino acid sequence at least 90, 91, 92, 93, 94, 95, 96, 97, 98 or 99% identical to SEQ ID NO: 64;
(4) a heavy chain variable region (VH) comprising an amino acid sequence at least 90, 91, 92, 93, 94, 95, 96, 97, 98 or 99% identical to SEQ ID NO: 14, or SEQ ID NO: 74, and a light chain variable region (VL) comprising an amino acid sequence at least 90, 91, 92, 93, 94, 95, 96, 97, 98 or 99% identical to SEQ ID NO: 64;
(5) a heavy chain variable region (VH) comprising an amino acid sequence at least 90, 91, 92, 93, 94, 95, 96, 97, 98 or 99% identical to SEQ ID NO: 19, or SEQ ID NO: 75, and a light chain variable region (VL) comprising an amino acid sequence at least 90, 91, 92, 93, 94, 95, 96, 97, 98 or 99% identical to SEQ ID NO: 64;
(6) a heavy chain variable region (VH) comprising an amino acid sequence at least 90, 91, 92, 93, 94, 95, 96, 97, 98 or 99% identical to SEQ ID NO: 24, or SEQ ID NO: 76, and a light chain variable region (VL) comprising an amino acid sequence at least 90, 91, 92, 93, 94, 95, 96, 97, 98 or 99% identical to SEQ ID NO: 64;
(7) a heavy chain variable region (VH) comprising an amino acid sequence at least 90, 91, 92, 93, 94, 95, 96, 97, 98 or 99% identical to SEQ ID NO: 29, or SEQ ID NO: 77, and a light chain variable region (VL) comprising an amino acid sequence at least 90, 91, 92, 93, 94, 95, 96, 97, 98 or 99% identical to SEQ ID NO: 64;
(8) a heavy chain variable region (VH) comprising an amino acid sequence at least 90, 91, 92, 93, 94, 95, 96, 97, 98 or 99% identical to SEQ ID NO: 34, or SEQ ID NO: 78, and a light chain variable region (VL) comprising an amino acid sequence at least 90, 91, 92, 93, 94, 95, 96, 97, 98 or 99% identical to SEQ ID NO: 64;
(9) a heavy chain variable region (VH) comprising an amino acid sequence at least 90, 91, 92, 93, 94, 95, 96, 97, 98 or 99% identical to SEQ ID NO: 39, or SEQ ID NO: 79, and a light chain variable region (VL) comprising an amino acid sequence at least 90, 91, 92, 93, 94, 95, 96, 97, 98 or 99% identical to SEQ ID NO: 64;
(10) a heavy chain variable region (VH) comprising an amino acid sequence at least 90, 91, 92, 93, 94, 95, 96, 97, 98 or 99% identical to SEQ ID NO: 44, or SEQ ID NO: 80, and a light chain variable region (VL) comprising an amino acid sequence at least 90, 91, 92, 93, 94, 95, 96, 97, 98 or 99% identical to SEQ ID NO: 64;
(11) a heavy chain variable region (VH) comprising an amino acid sequence at least 90, 91, 92, 93, 94, 95, 96, 97, 98 or 99% identical to SEQ ID NO: 49, or SEQ ID NO: 81, and a light chain variable region (VL) comprising an amino acid sequence at least 90, 91, 92, 93, 94, 95, 96, 97, 98 or 99% identical to SEQ ID NO: 64; or
(12) a heavy chain variable region (VH) comprising an amino acid sequence at least 90, 91, 92, 93, 94, 95, 96, 97, 98 or 99% identical to SEQ ID NO: 144, and a light chain variable region (VL) comprising an amino acid sequence at least 90, 91, 92, 93, 94, 95, 96, 97, 98 or 99% identical to SEQ ID NO: 64.

In some embodiments, the first antigen binding domain comprises a heavy chain variable region (VH) comprising the amino acid sequence of: SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, SEQ ID NO: 144, or SEQ ID NO: 64 wherein one, two, three, four, five, six, seven, eight, nine, or ten amino acid residues have been inserted, deleted or substituted (optionally conservative amino acid substitutions), while retaining therapeutic activity/binding specificity/affinity, optionally wherein the corresponding sequences of CDRs do not change.

In some embodiments, the first antigen binding domain comprises a heavy chain variable region (VH) comprising the amino acid sequence of: SEQ ID NO: 85 and SEQ ID NO: 64, SEQ ID NO: 87 and SEQ ID NO: 64, SEQ ID NO: 89 and SEQ ID NO: 64, SEQ ID NO: 91 and SEQ ID NO: 64, SEQ ID NO: 93 and SEQ ID NO: 64, SEQ ID NO: 4 and SEQ ID NO: 64, SEQ ID NO: 72 and SEQ ID NO: 64, SEQ ID NO: 9 and SEQ ID NO: 64, SEQ ID NO: 73 and SEQ ID NO: 64, SEQ ID NO: 14 and SEQ ID NO: 64, SEQ ID NO: 74 and SEQ ID NO: 64, SEQ ID NO: 19 and SEQ ID NO: 64, SEQ ID NO: 75 and SEQ ID NO: 64, SEQ ID NO: 24 and SEQ ID NO: 64, SEQ ID NO: 76 and SEQ ID NO: 64, SEQ ID NO: 29 and SEQ ID NO: 64, SEQ ID NO: 77 and SEQ ID NO: 64, SEQ ID NO: 34 and SEQ ID NO: 64, SEQ ID NO: 78 and SEQ ID NO: 64, SEQ ID NO: 39 and SEQ ID NO: 64, SEQ ID NO: 79 and SEQ ID NO: 64, SEQ ID NO: 44 and SEQ ID NO: 64, SEQ ID NO: 80 and SEQ ID NO: 64, SEQ ID NO: 49 and SEQ ID NO: 64, SEQ ID NO: 81 and SEQ ID NO: 64, or SEQ ID NO: 144 and SEQ ID NO: 64 wherein one, two, three, four, five, six, seven, eight, nine, or ten amino acid residues have been inserted, deleted or substituted (optionally conservative amino acid substitutions), while retaining therapeutic activity/binding specificity/affinity, optionally wherein the corresponding sequences of CDRs do not change.

In some embodiments, the first antigen binding domain comprises:
(1) a heavy chain variable region (VH) comprising an amino acid sequence of SEQ ID NO: 85, SEQ ID NO: 87, SEQ ID NO: 89, SEQ ID NO: 91, or SEQ ID NO: 93, and a light chain variable region (VL) comprising an amino acid sequence of SEQ ID NO: 64;
(2) a heavy chain variable region (VH) comprising an amino acid sequence of SEQ ID NO: 4 or SEQ ID NO: 72, and a light chain variable region (VL) comprising an amino acid sequence of SEQ ID NO: 64;
(3) a heavy chain variable region (VH) comprising an amino acid sequence of SEQ ID NO: 9 or SEQ ID NO: 73, and a light chain variable region (VL) comprising an amino acid sequence of SEQ ID NO: 64;
(4) a heavy chain variable region (VH) comprising an amino acid sequence of SEQ ID NO: 14, or SEQ ID NO: 74, and a light chain variable region (VL) comprising an amino acid sequence of SEQ ID NO: 64;
(5) a heavy chain variable region (VH) comprising an amino acid sequence of SEQ ID NO: 19, or SEQ ID NO: 75, and a light chain variable region (VL) comprising an amino acid sequence of SEQ ID NO: 64;
(6) a heavy chain variable region (VH) comprising an amino acid sequence of SEQ ID NO: 24, or SEQ ID NO: 76, and a light chain variable region (VL) of SEQ ID NO: 64;
(7) a heavy chain variable region (VH) comprising an amino acid sequence of SEQ ID NO: 29, or SEQ ID NO: 77, and a light chain variable region (VL) comprising an amino acid sequence of SEQ ID NO: 64;
(8) a heavy chain variable region (VH) comprising an amino acid sequence of SEQ ID NO: 34, or SEQ ID NO: 78, and a light chain variable region (VL) comprising an amino acid sequence of SEQ ID NO: 64;
(9) a heavy chain variable region (VH) comprising an amino acid sequence of SEQ ID NO: 39, or SEQ ID NO: 79, and a light chain variable region (VL) comprising an amino acid sequence of SEQ ID NO: 64;
(10) a heavy chain variable region (VH) comprising an amino acid sequence of SEQ ID NO: 44, or SEQ ID NO: 80, and a light chain variable region (VL) comprising an amino acid sequence of SEQ ID NO: 64; or
(11) a heavy chain variable region (VH) comprising an amino acid sequence of SEQ ID NO: 49, or SEQ ID NO: 81, and a light chain variable region (VL) comprising an amino acid sequence of SEQ ID NO: 64; or
(12) a heavy chain variable region (VH) comprising an amino acid sequence of SEQ ID NO: 144, and a light chain variable region (VL) comprising an amino acid sequence of SEQ ID NO: 64.

Other first antigen binding domains of the conjugates of the present disclosure include amino acids that have been changed, yet have at least 60%, at least 70%, at least 80%, at least 90%, at least 91%, at least 92%, at least 93%, at least 94%, at least 95% at least 96%, at least 97%, at least 98%, or at least 99% percent identity in the CDR regions compared with the CDR regions disclosed in Table 1. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 1, while maintaining binding specificity and affinity.

Other first antigen binding domains of the present disclosure include those where the amino acids or nucleic acids encoding the amino acids have been changed in the variable regions (e.g., the frameworks regions of the variable regions); yet have at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% percent identity to the sequences of variable regions described in Table 1, while retaining binding specificity/affinity, optionally the corresponding sequences of CDRs do not change. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., the frameworks regions of the variable regions) when compared with the variable regions depicted in the sequence described in Table 1, while retaining binding specificity/affinity, optionally the corresponding sequences of CDRs do not change. The changes could be insertion, deletion or substitution, optionally conservative amino acid substitutions.

In another embodiment, the present disclosure provides for conjugates made from antibodies or antigen-binding fragments thereof that specifically bind to human cMET with a binding affinity (K_{D}) of from 1×10⁻⁶ M to 1×10⁻¹¹ M. In another embodiment, the anti-cMET antibodies or antigen-binding fragments thereof bind to human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M or about 1×10⁻¹¹ M.

The present disclosure also provides nucleic acid sequences that encode VH or VL of the antibodies used in the conjugates that specifically bind to human cMET. Such nucleic acid sequences can be optimized for expression in mammalian cells.

The present disclosure also provides for first antigen binding domains that bind to the same epitope as do the anti-cMET antibodies described in Table 1. Additional first antigen binding domains can therefore be identified based on their ability to cross-compete (e.g., to competitively inhibit the binding of, in a statistically significant manner) with the antibodies described in Table 1 in binding assays. The ability of a test antibody to inhibit the first antigen binding domains of the present disclosure to human cMET demonstrates that the test antibody can compete with that antibody or antigen-binding fragments thereof for binding to human cMET. Such an antibody can, without being bound to any one theory, bind to the same or a related (e.g., a structurally similar or spatially proximal) epitope on human cMET as the antibody or antigen-binding fragments thereof with which it competes. In a certain aspect, a first antigen binding domain that binds to the same epitope on human cMET as the first antigen binding domains of the present disclosure is a human or humanized monoclonal antigen binding domain. Such antigen binding domains can be prepared and isolated as described herein.

In some embodiments, the first antigen binding domain is a monoclonal antibody, a human engineered antibody, a single chain antibody (scFv), a Fab fragment, a Fab′ fragment, or a F(ab′)2 fragment.

In one embodiment, the first antigen binding domain is in a scFv format comprising VH-VL in the direction of N-terminal to C-terminal, or VL-VH in the direction of N-terminal to C-terminal. In some embodiments, the VH and VL are connected via an amino acid linker described herein. In some embodiments, the VH comprises the amino acid sequence of any one of SEQ ID NO: 85, SEQ ID NO: 87, SEQ ID NO: 89, SEQ ID NO: 91, SEQ ID NO: 93, SEQ ID NO: 4, SEQ ID NO: 72, SEQ ID NO: 9, SEQ ID NO: 73, SEQ ID NO: 14, SEQ ID NO: 74, SEQ ID NO: 19, SEQ ID NO: 75, SEQ ID NO: 24, SEQ ID NO: 76, SEQ ID NO: 29, SEQ ID NO: 77, SEQ ID NO: 34, SEQ ID NO: 78, SEQ ID NO: 39, SEQ ID NO: 79, SEQ ID NO: 44, SEQ ID NO: 80, SEQ ID NO: 49, SEQ ID NO: 81, or SEQ ID NO: 144. In some embodiments, the VL comprises the amino acid sequence of any one of SEQ ID NO: 64. In some embodiments, the VH is any one of VHs described in Table 1. In some embodiment, the VL is any one of VLs described in Table 1. In some embodiments, the amino acid linker has an amino acid sequence comprising any one of SEQ ID NO: 97-139. In some embodiments, the amino acid linker is any sequence of SEQ ID NO: 97-139.

In some embodiments, the first antigen binding domain comprises a heavy chain sequence comprising a human IgG constant region. In some cases, the heavy chain constant region sequence comprises a human IgG1, IgG2, IgG3, or IgG4 constant region sequence. In some such cases, the heavy chain constant region sequence lacks the C-terminal lysine or the C-terminal glycine-lysine. In other cases, the C-terminal lysine or C-terminal glycine-lysine is present. In some cases, the heavy chain constant region comprises a knob substitution such as T366W, or a hole substitution such as T366S, L368A, and Y407V (all EU numbering). In some cases, the heavy chain constant region comprises a “YTE” substitution, comprising M252Y, S254T, and T256E. Further heavy chain constant region modifications that may be employed are discussed further below.

In some embodiments, the first antigen binding domain comprises a heavy chain constant region comprising the amino acid sequence of SEQ ID NO: 95 or SEQ ID NO: 96. In some embodiments, the first antigen binding domain comprises a heavy chain (HC) comprising the amino acid sequence of SEQ ID NO: 150, 169, 170, 171, or 172. In some embodiments, the first antigen binding domain comprises a light chain (LC) comprising the amino acid sequence of SEQ ID NO: 148.

### 1.2 Second Group of Anti-cMET Antigen Binding Domains

TABLE 2
Anti-cMET Monoclonal Antibody List 2

| | SEQ ID NO. | | | | | | | | | |
|---|---|---|---|---|---|---|---|---|---|---|
| | VH | VH | HCDR1 | HCDR2 | HCDR3 | VL | VL | LCDR1 | LCDR2 | LCDR3 |
| Antibody | (AA) | (DNA) | (Kabat) | (Kabat) | (Kabat) | (AA) | (DNA) | (Kabat) | (Kabat) | (Kabat) |
| 063Ab03210 | 54 | 55 | 56 | 57 | 58 | 64 | 65 | 66 | 67 | 68 |
| 063Ab03210- | 82 | / | 56 | 57 | 58 | 64 | 65 | 66 | 67 | 68 |
| V1 | | | | | | | | | | |
| BGA-032 | 145 | 163 | 56 | 57 | 58 | 64 | 65 | 66 | 67 | 68 |
| 063Ab16720 | 59 | 60 | 61 | 62 | 63 | 64 | 65 | 66 | 67 | 68 |
| 063Ab16720- | 83 | / | 61 | 62 | 63 | 64 | 65 | 66 | 67 | 68 |
| V1 | | | | | | | | | | |

The present disclosure provides for conjugates comprising a second antigen-binding domain that specifically binds to a second epitope of human cMET, wherein the first and second epitopes of human cMET are distinct, or wherein the first antigen binding domain does not compete with the second antigen binding domain. Exemplary sequences of second antigen binding domains are provided in Table 2. In some cases, the second antigen binding domain comprises a VH domain comprising an amino acid sequence of SEQ ID NO: 54, SEQ ID NO: 82, SEQ ID NO: 145, SEQ ID NO: 59, or SEQ ID NO: 83 (Table 2). In some embodiments, the second antigen binding domain comprises a HCDR comprising an amino acid sequence of any one of the HCDRs listed in Table 2. In one aspect, the second antigen binding domain comprises one, two, or three HCDRs comprising an amino acid sequence of any of the HCDRs listed in Table 2.

The present disclosure provides for second antigen binding domains that comprise a VL domain having an amino acid sequence of SEQ ID NO: 64 (Table 2). The present disclosure also provides second antigen binding domains that comprise a LCDR comprising an amino acid sequence of any one of the LCDRs listed in Table 2. In one aspect, the second antigen binding domain comprises one, two, or three LCDRs comprising an amino acid sequence of any of the LCDRs listed in Table 2.

In one embodiment, the second antigen binding domain comprises one or more complementarity determining regions (CDRs) comprising an amino acid sequence selected from a group consisting of SEQ ID NO: 56, SEQ ID NO: 57, SEQ ID NO: 58, SEQ ID NO: 61, SEQ ID NO: 62; SEQ ID NO: 63, SEQ ID NO: 66, SEQ ID NO: 67 and SEQ ID NO: 68.

In another embodiment, the second antigen binding domain comprises: (a) a heavy chain variable region comprising one or more complementarity determining regions (HCDRs) comprising an amino acid sequence selected from the group consisting of SEQ ID NO: 56, SEQ ID NO: 57, SEQ ID NO: 58, SEQ ID NO: 61, SEQ ID NO: 62; SEQ ID NO: 63, and/or (b) a light chain variable region comprising one or more complementarity determining regions (LCDRs) comprising an amino acid sequence selected from the group consisting of SEQ ID NO: 66, SEQ ID NO: 67 and SEQ ID NO: 68.

In another embodiment, the second antigen binding domain comprises: (a) a heavy chain variable region comprising three complementarity determining regions (HCDRs) which are HCDR1 comprising an amino acid sequence of SEQ ID NO: 56, or SEQ ID NO: 61; HCDR2 comprising an amino acid sequence of SEQ ID NO: 57, or SEQ ID NO: 62; and HCDR3 comprising an amino acid sequence of SEQ ID NO: 58, or SEQ ID NO: 63; and/or (b) a light chain variable region comprising three complementarity determining regions (LCDRs) which are LCDR1 comprising an amino acid sequence of SEQ ID NO: 66; LCDR2 comprising an amino acid sequence of SEQ ID NO: 67; and LCDR3 comprising an amino acid sequence of SEQ ID NO: 68.

In another embodiment, the second antigen binding domain comprises: (a) a heavy chain variable region comprising three complementarity determining regions (HCDRs) which are: HCDR1 comprising an amino acid sequence of SEQ ID NO: 56, HCDR2 comprising an amino acid sequence of SEQ ID NO: 57, and HCDR3 comprising an amino acid sequence of SEQ ID NO: 58; or HCDR1 comprising an amino acid sequence of SEQ ID NO: SEQ ID NO: 61, HCDR2 comprising an amino acid sequence of SEQ ID NO: 62, and HCDR3 comprising an amino acid sequence of SEQ ID NO: 63, and/or (b) a light chain variable region comprising three complementarity determining regions (LCDRs) which are LCDR1 comprising an amino acid sequence of SEQ ID NO: 66, LCDR2 comprising an amino acid sequence of SEQ ID NO: 67, and LCDR3 comprising an amino acid sequence of SEQ ID NO: 68.

In one embodiment, the second antigen binding domain comprises: (1) a HCDR1 (Heavy Chain Complementarity Determining Region 1), a HCDR2 and a HCDR3 from the heavy chain variable region (VH) set forth in SEQ ID NO: 54, SEQ ID NO: 82, or SEQ ID NO: 145; (2) a HCDR1, a HCDR2 and a HCDR3 from the heavy chain variable region (VH) set forth in SEQ ID NO: 59 or SEQ ID NO: 83; and/or (1) a LCDR1 (Light Chain Complementarity Determining Region 1), a LCDR2 and a LCDR3 from the light chain variable region (VL) set forth in SEQ ID NO: 64.

In one embodiment, the second antigen binding domain comprises:
(1) a heavy chain variable region (VH) that comprises (a) a HCDR1 (Heavy Chain Complementarity Determining Region 1) of SEQ ID NO: 56, (b) a HCDR2 of SEQ ID NO: 57, (c) a HCDR3 of SEQ ID NO: 58 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region (VH) that comprises (a) a HCDR1 (Heavy Chain Complementarity Determining Region 1) of SEQ ID NO: 61, (b) a HCDR2 of SEQ ID NO: 62, (c) a HCDR3 of SEQ ID NO: 63 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; according to the Kabat definition.

In one embodiment, the second antigen binding domain comprises: (a) a heavy chain variable region comprising an amino acid sequence of SEQ ID NO: 54, SEQ ID NO: 82, SEQ ID NO: 145, SEQ ID NO: 59, or SEQ ID NO: 83; or an amino acid sequence at least 90%, 91%, 92%, 93%, 94%, 95%, 96%, 97%, 98% or 99% identical to any one of SEQ ID NO: 54, SEQ ID NO: 82, SEQ ID NO: 145, SEQ ID NO: 59, or SEQ ID NO: 83 and/or (b) a light chain variable region comprising an amino acid sequence of SEQ ID NO: 64, or an amino acid sequence at least 90%, 91%, 92%, 93%, 94%, 95%, 96%, 97%, 98% or 99% identical to SEQ ID NO: 64.

In one embodiment, the second antigen binding domain comprises:
(1) a heavy chain variable region (VH) comprising an amino acid sequence at least 90, 91, 92, 93, 94, 95, 96, 97, 98 or 99% identical to SEQ ID NO: 54, or SEQ ID NO: 82, and a light chain variable region (VL) comprising an amino acid sequence at least 90, 91, 92, 93, 94, 95, 96, 97, 98 or 99% identical to SEQ ID NO: 64;
(2) a heavy chain variable region (VH) comprising an amino acid sequence at least 90, 91, 92, 93, 94, 95, 96, 97, 98 or 99% identical to SEQ ID NO: 59 or SEQ ID NO: 83, and a light chain variable region (VL) comprising an amino acid sequence at least 90, 91, 92, 93, 94, 95, 96, 97, 98 or 99% identical to SEQ ID NO: 64; or
(3) a heavy chain variable region (VH) comprising an amino acid sequence at least 90, 91, 92, 93, 94, 95, 96, 97, 98 or 99% identical to SEQ ID NO: 145, and a light chain variable region (VL) comprising an amino acid sequence at least 90, 91, 92, 93, 94, 95, 96, 97, 98 or 99% identical to SEQ ID NO: 64.

In one embodiment, the second antigen binding domain comprises the amino acid sequence of SEQ ID NO: 54, SEQ ID NO: 82, SEQ ID NO: 145, SEQ ID NO: 59, or SEQ ID NO: 83, or SEQ ID NO: 64 wherein one, two, three, four, five, six, seven, eight, nine, or ten amino acid residues have been inserted, deleted or substituted (optionally conservative amino acid substitutions), while retaining therapeutic activity/binding specificity/affinity, optionally wherein the corresponding sequences of CDRs do not change.

In one embodiment, the second antigen binding domain comprises the amino acid sequence of, wherein SEQ ID NO: 54 and SEQ ID NO: 64, SEQ ID NO: 82 and SEQ ID NO: 64, SEQ ID NO: 145 and SEQ ID NO: 64, SEQ ID NO: 59 and SEQ ID NO: 64, or SEQ ID NO: 83 and SEQ ID NO: 64 wherein one, two, three, four, five, six, seven, eight, nine, or ten amino acids have been inserted, deleted or substituted (optionally conservative amino acid substitutions), while retaining therapeutic activity/binding specificity/affinity, optionally wherein the corresponding sequences of CDRs do not change.

In one embodiment, the second antigen binding domain comprises:
(1) a heavy chain variable region (VH) comprising an amino acid sequence of SEQ ID NO: 54, or SEQ ID NO: 82, and a light chain variable region (VL) comprising an amino acid sequence of SEQ ID NO: 64;
(2) a heavy chain variable region (VH) comprising an amino acid sequence of SEQ ID NO: 59 or SEQ ID NO: 83, and a light chain variable region (VL) comprising an amino acid sequence of SEQ ID NO: 64; or
(3) a heavy chain variable region (VH) comprising an amino acid sequence of SEQ ID NO: 145, and a light chain variable region (VL) comprising an amino acid sequence of SEQ ID NO: 64.

Other second antigen binding domains of the present disclosure include amino acids that have been changed, yet have at least 60%, at least 70%, at least 80%, at least 90%, at least 91%, at least 92%, at least 93%, at least 94%, at least 95% at least 96%, at least 97%, at least 98%, or at least 99% percent identity in the CDR regions compared with the CDR regions disclosed in Table 2. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 2, while maintaining binding specificity and affinity.

Other second binding domains of the present disclosure include those where the amino acids or nucleic acids encoding the amino acids have been changed in the variable regions (e.g., the frameworks regions of the variable regions); yet have at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the sequences of variable regions described in Table 2, while retaining binding specificity/affinity, optionally the corresponding sequences of CDRs do not change. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., the frameworks regions of the variable regions) when compared with the variable regions depicted in the sequence described in Table 2, while retaining binding specificity/affinity, optionally the corresponding sequences of CDRs do not change. The changes could be insertion, deletion or substitution, optionally conservative amino acid substitutions.

In another embodiment, the present disclosure provides for second binding domains derived from antibodies or antigen-binding fragments thereof that specifically bind to human cMET with a binding affinity (K_{D}) of from 1×10⁻⁶ M to 1×10⁻¹¹ M. In another embodiment, the anti-cMET antibodies or antigen-binding fragments thereof bind to human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M or about 1×10⁻¹¹ M.

The present disclosure also provides nucleic acid sequences that encode VH or VL of the second antigen binding domains that specifically bind to human cMET. Such nucleic acid sequences can be optimized for expression in mammalian cells.

The present disclosure also provides for other second antigen binding domains that bind to the same epitope as do the anti-cMET antibodies described in Table 2. Additional second antigen binding domains can therefore be identified based on their ability to cross-compete (e.g., to competitively inhibit the binding of, in a statistically significant manner) with the antibodies described in Table 2 in binding assays. The ability of a test antibody or binding domain to inhibit the binding domains of the present disclosure to human cMET demonstrates that the test antibody or binding domain can compete with that domain for binding to human cMET. Such an antibody can, without being bound to any one theory, bind to the same or a related (e.g., a structurally similar or spatially proximal) epitope on human cMET as the antibody or antigen binding domain with which it competes. In a certain aspect, an antibody or antigen binding domain that binds to the same epitope on human cMET as the second binding domains of the present disclosure is a human or humanized monoclonal antibody.

In some embodiments, the second antigen binding domain is a monoclonal antibody, a human engineered antibody, a single chain antibody (scFv), a Fab fragment, a Fab′ fragment, or a F(ab′)2 fragment.

In one embodiment, the second antigen binding domain is in a scFv format comprising VH-VL in the direction of N-terminal to C-terminal, or VL-VH in the direction of N-terminal to C-terminal. In some embodiments, the VH and VL are connected via an amino acid linker described herein.

In some embodiments, the VH of the second antigen binding domain comprises the amino acid sequence of any one of SEQ ID NO: 54, SEQ ID NO: 82, SEQ ID NO: 145, SEQ ID NO: 59, or SEQ ID NO: 83. In some embodiments, the VL comprises the amino acid sequence of any one of SEQ ID NO: 64. In some embodiments, the VH is any one of VHs described in Table 2. In some embodiment, the VL is any one of VLs described in Table 2. In some embodiments, the amino acid linker has an amino acid sequence comprising any one of SEQ ID NO: 97-139. In some embodiments, the amino acid linker is any sequence of SEQ ID NO: 97-139.

In some embodiments, the second antigen binding domain comprises a heavy chain sequence comprising a human IgG constant region. In some cases, the heavy chain constant region sequence comprises a human IgG1, IgG2, IgG3, or IgG4 constant region sequence. In some such cases, the heavy chain constant region sequence lacks the C-terminal lysine or the C-terminal glycine-lysine. In other cases, the C-terminal lysine or C-terminal glycine-lysine is present. In some cases, the heavy chain constant region comprises a knob substitution such as T366W, or a hole substitution such as T366S, L368A, and Y407V (all EU numbering). In some cases, the heavy chain constant region comprises a “YTE” substitution, comprising M252Y, S254T, and T256E. Further heavy chain constant region modifications that may be employed are discussed further below.

In some embodiments, the second antigen binding domain comprises a heavy chain constant region comprising the amino acid sequence of SEQ ID NO: 95 or SEQ ID NO: 96. In some embodiments, the second antigen binding domain comprises a heavy chain (HC) comprising the amino acid sequence of SEQ ID NO: 153 or 168. In some embodiments, the second antigen binding domain comprises a light chain (LC) comprising the amino acid sequence of SEQ ID NO: 148.

### II. Anti-EGFR Antigen Binding Domains

The present disclosure provides for third antigen binding domains that specifically bind to human EGFR. In one embodiment, the third antigen binding domains are derived from anti-EGFR antibodies or antigen-binding fragments thereof specifically bind to human EGFR with a binding affinity (K_{D}) of from 1×10⁻⁶ M to 1×10⁻¹⁰ M. In another embodiment, the anti-EGFR antibodies or antigen-binding fragments thereof bind to human EGFR with a binding affinity (K_{D}) of about 1×10⁻⁶ M, about 1×10⁻⁷ M, about 1×10⁻⁸ M, about 1×10⁻⁹ M or about 1×10⁻¹⁰ M.

In one embodiment, the third antigen binding domains comprise: a heavy chain variable region (VH) that comprises (a) a HCDR1 of SEQ ID NO: 155, (b) a HCDR2 of SEQ ID NO: 156, (c) a HCDR3 of SEQ ID NO: 157; and a light chain variable region (VL) that comprises (d) a LCDR1 of SEQ ID NO: 158, (e) a LCDR2 of SEQ ID NO: 159, (f) a LCDR3 of SEQ ID NO: 160, according to the Kabat numbering.

In another embodiment, the third antigen binding domains comprise: a HCDR1, a HCDR2 and a HCDR3 from the heavy chain variable region (VH) set forth in SEQ ID NO: 142; and a LCDR1, a LCDR2 and a LCDR3 from the light chain variable region (VL) set forth in SEQ ID NO: 143.

In another embodiment, the third antigen binding domains further comprise no more than one, two, three, four or five amino acid deletions, insertions or substitutions in the CDR, preferably the amino acid substitutions are conservative amino acid substitutions, while maintaining binding specificity and affinity.

In another embodiment, the third antigen binding domains comprise a heavy chain variable region (VH) comprising an amino acid sequence at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 142, and a light chain variable region (VL) comprising an amino acid sequence at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 143. In another embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids within SEQ ID NO: 142 or SEQ ID NO: 143 have been inserted, deleted or substituted (optionally conservative amino acid substitutions). In another embodiment, such variations are in the framework region of the variable region. In another embodiment, anti-EGFR antibodies or antigen-binding fragments thereof having such variations maintains binding specificity and affinity.

In another embodiment, the third antigen binding domains comprise a heavy chain variable region (VH) that comprises SEQ ID NO: 142, and a light chain variable region (VL) that comprises SEQ ID NO: 143.

In one embodiment, the third antigen binding domain is in a scFv format comprising VH-VL in the direction of N-terminal to C-terminal, or VL-VH in the direction of N-terminal to C-terminal. In some embodiments, the VH and VL are connected via an amino acid linker described herein. In some embodiments, the VH comprises the amino acid sequence of SEQ ID NO: 142. In some embodiments, the VL comprises the amino acid sequence of SEQ ID NO: 143. In some embodiments, the amino acid linker has an amino acid sequence comprising any one of SEQ ID NO: 97-139. In some embodiments, the amino acid linker is any sequence of SEQ ID NO: 97-139. In one embodiment, the amino acid linker is SEQ ID NO: 139.

In one embodiment, the third antigen binding domain comprises a scFv having the amino acid sequence of SEQ ID NO: 161. In another embodiment, the antibody or antigen-binding fragment thereof comprises a scFv of SEQ ID NO: 161.

### III. Multispecific Antibodies

### 3.1 Anti-cMET Bispecific Antibodies

The present disclosure provides for bispecific antibodies or antigen-binding fragments that specifically bind to human cMET, comprising a first antigen binding domain that specifically binds a first epitope of human cMET, and a second antigen binding domain that specifically binds to a second epitope of human cMET, wherein the first antigen binding domain is distinct from the second antigen binding domain. In other words, in some embodiments, a first antigen binding domain that specifically binds a first epitope of human cMET, and a second antigen binding domain that specifically binds to a second epitope of human cMET collectively comprise a bispecific antibody.

In one embodiment, the first epitope is distinct from the second epitope (e.g., non-overlapping), or the first antigen binding domain does not compete with the second antigen binding domain.

In one aspect, the first antigen binding domain that specifically binds the first epitope of human cMET and the second antigen binding domain that specifically binds the second epitope of human cMET are selected from any anti-cMET antibody described in Section I and are distinct from each other.

In one aspect, the first antigen binding domain that specifically binds the first epitope of human cMET and the second antigen binding domain that specifically binds the second epitope of human cMET are selected from any anti-cMET antibody described in Section I and do not compete with each other.

In another aspect, the first antigen binding domain that specifically binds the first epitope of human cMET could be any antibody or antigen-binding fragment selected from those described in Section 1.1 “first group of anti-cMET antibodies” including Table 1. The second antigen binding domain that specifically binds the second epitope of human cMET could be any antibody or antigen-binding fragment selected from those described in Section 1.2 “second group of anti-cMET antibodies” including Table 2.

In some embodiments, the bispecific antibody comprises antigen binding fragments, wherein the antigen-binding fragment can be a Fab, F(ab′)₂, Fv, a single chain Fv (scFv), or a single domain antibody.

In some embodiments, the bispecific antibody comprises an IgG heavy chain constant region, i.e., comprising an Fc domain, and/or a human kappa or lambda light chain constant region. In some embodiments, the bispecific antibody comprises a human IgG1, IgG2, IgG3, or IgG4 constant region. In some such cases, the heavy chain constant region sequence lacks the C-terminal lysine or the C-terminal glycine-lysine. In other cases, the C-terminal lysine or C-terminal glycine-lysine is present. In some cases, the heavy chain constant region comprises “knob into hole” substitutions such as T366W on one heavy chain constant region and T366S, L368A, and Y407V substitutions on the other heavy chain constant region (all EU numbering). In some cases, the heavy chain constant region comprises a “YTE” substitution on one or both heavy chains, comprising M252Y, S254T, and T256E. Further heavy chain constant region modifications that may be employed are discussed further below.

In one embodiment, the bispecific antibody of the present disclosure binds to human cMET with a binding affinity (K_{D}) of from 1×10⁻⁶ M to 1×10⁻¹⁰ M, or even 1×10⁻¹¹ M. In another embodiment, the bispecific antibody of the present disclosure binds to human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

In some embodiments, the bispecific antibodies of the present disclosure specifically binding to two distinct epitopes (e.g., non-overlapping or non-competing) of human cMET exhibit better blocking activity on ligand independent signaling and/or ligand induced signaling, compared with those of the antibodies comprising only one antigen binding domain that specifically binds to human cMET.

In one embodiment, the present disclosure provides for a bispecific antibody or antigen-binding fragment thereof, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 94 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises (a) a HCDR1 of SEQ ID NO: 6, (b) a HCDR2 of SEQ ID NO: 7, (c) a HCDR3 of SEQ ID NO: 8 and a light chain variable region that comprises: (d) a LCDR1 of SEQ ID NO: 66, (e) a LCDR2 of SEQ ID NO: 67, and (f) a LCDR3 of SEQ ID NO: 68;
(3) a heavy chain variable region that comprises (a) a HCDR1 of SEQ ID NO: 11, (b) a HCDR2 of SEQ ID NO: 12, (c) a HCDR3 of SEQ ID NO: 13 and a light chain variable region that comprises: (d) a LCDR1 of SEQ ID NO: 66, (e) a LCDR2 of SEQ ID NO: 67, and (f) a LCDR3 of SEQ ID NO: 68;
(4) a heavy chain variable region that comprises (a) a HCDR1 of SEQ ID NO: 16, (b) a HCDR2 of SEQ ID NO: 17, (c) a HCDR3 of SEQ ID NO: 18 and a light chain variable region that comprises: (d) a LCDR1 of SEQ ID NO: 66, (e) a LCDR2 of SEQ ID NO: 67, and (f) a LCDR3 of SEQ ID NO: 68;
(5) a heavy chain variable region that comprises (a) a HCDR1 of SEQ ID NO: 21, (b) a HCDR2 of SEQ ID NO: 22, (c) a HCDR3 of SEQ ID NO: 23 and a light chain variable region that comprises: (d) a LCDR1 of SEQ ID NO: 66, (e) a LCDR2 of SEQ ID NO: 67, and (f) a LCDR3 of SEQ ID NO: 68;
(6) a heavy chain variable region that comprises (a) a HCDR1 of SEQ ID NO: 26, (b) a HCDR2 of SEQ ID NO: 27, (c) a HCDR3 of SEQ ID NO: 28 and a light chain variable region that comprises: (d) a LCDR1 of SEQ ID NO: 66, (e) a LCDR2 of SEQ ID NO: 67, and (f) a LCDR3 of SEQ ID NO: 68;
(7) a heavy chain variable region that comprises (a) a HCDR1 of SEQ ID NO: 31, (b) a HCDR2 of SEQ ID NO: 32, (c) a HCDR3 of SEQ ID NO: 33 and a light chain variable region that comprises: (d) a LCDR1 of SEQ ID NO: 66, (e) a LCDR2 of SEQ ID NO: 67, and (f) a LCDR3 of SEQ ID NO: 68;
(8) a heavy chain variable region that comprises (a) a HCDR1 of SEQ ID NO: 36, (b) a HCDR2 of SEQ ID NO: 37, (c) a HCDR3 of SEQ ID NO: 38 and a light chain variable region that comprises: (d) a LCDR1 of SEQ ID NO: 66, (e) a LCDR2 of SEQ ID NO: 67, and (f) a LCDR3 of SEQ ID NO: 68;
(9) a heavy chain variable region that comprises (a) a HCDR1 of SEQ ID NO: 41, (b) a HCDR2 of SEQ ID NO: 42, (c) a HCDR3 of SEQ ID NO: 43 and a light chain variable region that comprises: (d) a LCDR1 of SEQ ID NO: 66, (e) a LCDR2 of SEQ ID NO: 67, and (f) a LCDR3 of SEQ ID NO: 68;
(10) a heavy chain variable region that comprises (a) a HCDR1 of SEQ ID NO: 46, (b) a HCDR2 of SEQ ID NO: 47, (c) a HCDR3 of SEQ ID NO: 48 and a light chain variable region that comprises: (d) a LCDR1 of SEQ ID NO: 66, (e) a LCDR2 of SEQ ID NO: 67, and (f) a LCDR3 of SEQ ID NO: 68; or
(11) a heavy chain variable region that comprises (a) a HCDR1 of SEQ ID NO: 51, (b) a HCDR2 of SEQ ID NO: 52, (c) a HCDR3 of SEQ ID NO: 53 and a light chain variable region that comprises: (d) a LCDR1 of SEQ ID NO: 66, (e) a LCDR2 of SEQ ID NO: 67, and (f) a LCDR3 of SEQ ID NO: 68; and
wherein the second antigen binding domain that specifically binds to the second epitope of human cMET comprises: a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68.

In one embodiment, the present disclosure provides for a bispecific antibody or antigen-binding fragment thereof, wherein the first antigen binding domain that specifically binds to the first epitope of human cMET comprises:
(1) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 84 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 86 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(3) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 88 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(4) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 90 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; or
(5) a heavy chain variable region (VH) that comprises (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68; and
wherein the second antigen binding domain that specifically binds to the second epitope of human cMET comprises: a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68.

In one embodiment, the present disclosure provides for a bispecific antibody or antigen-binding fragment thereof, wherein the first antigen binding domain that specifically binds to the first epitope of human cMET comprises:
(1) a heavy chain variable region (VH) comprising an amino acid sequence of SEQ ID NO: 4 or SEQ ID NO: 72, and a light chain variable region (VL) comprising an amino acid sequence of SEQ ID NO: 64;
(2) a heavy chain variable region (VH) comprising an amino acid sequence of SEQ ID NO: 9 or SEQ ID NO: 73, and a light chain variable region (VL) comprising an amino acid sequence of SEQ ID NO: 64;
(3) a heavy chain variable region (VH) comprising an amino acid sequence of SEQ ID NO: 14, or SEQ ID NO: 74, and a light chain variable region (VL) comprising an amino acid sequence of SEQ ID NO: 64;
(4) a heavy chain variable region (VH) comprising an amino acid sequence of SEQ ID NO: 19, or SEQ ID NO: 75, and a light chain variable region (VL) comprising an amino acid sequence of SEQ ID NO: 64;
(5) a heavy chain variable region (VH) comprising an amino acid sequence of SEQ ID NO: 24, or SEQ ID NO: 76, and a light chain variable region (VL) of SEQ ID NO: 64;
(6) a heavy chain variable region (VH) comprising an amino acid sequence of SEQ ID NO: 29, or SEQ ID NO: 77, and a light chain variable region (VL) comprising an amino acid sequence of SEQ ID NO: 64;
(7) a heavy chain variable region (VH) comprising an amino acid sequence of SEQ ID NO: 34, or SEQ ID NO: 78, and a light chain variable region (VL) comprising an amino acid sequence of SEQ ID NO: 64;
(8) a heavy chain variable region (VH) comprising an amino acid sequence of SEQ ID NO: 39, or SEQ ID NO: 79, and a light chain variable region (VL) comprising an amino acid sequence of SEQ ID NO: 64;
(9) a heavy chain variable region (VH) comprising an amino acid sequence of SEQ ID NO: 44, or SEQ ID NO: 80, and a light chain variable region (VL) comprising an amino acid sequence of SEQ ID NO: 64; or
(10) a heavy chain variable region (VH) comprising an amino acid sequence of SEQ ID NO: 49 or SEQ ID NO: 81, and a light chain variable region (VL) comprising an amino acid sequence of SEQ ID NO: 64; and
wherein the second antigen binding domain that specifically binds to the second epitope of human cMET comprises:
a heavy chain variable region (VH) comprising an amino acid sequence of SEQ ID NO: 54, SEQ ID NO: 82 or SEQ ID NO: 145, and a light chain variable region (VL) comprising an amino acid sequence of SEQ ID NO: 64.

In one embodiment, the present disclosure provides for a bispecific antibody or antigen-binding fragment thereof, wherein the first antigen binding domain that specifically binds to the first epitope of human cMET comprises
a heavy chain variable region (VH) comprising an amino acid sequence of SEQ ID NO: 85, SEQ ID NO: 87, SEQ ID NO: 89, SEQ ID NO: 91, SEQ ID NO: 93, or SEQ ID NO: 144, and a light chain variable region (VL) comprising an amino acid sequence of SEQ ID NO: 64; and
wherein the second antigen binding domain that specifically binds to the second epitope of human cMET comprises:
a heavy chain variable region (VH) comprising an amino acid sequence of SEQ ID NO: 54, SEQ ID NO: 82 or SEQ ID NO: 145, and a light chain variable region (VL) comprising an amino acid sequence of SEQ ID NO: 64.

In some embodiments, in the above-mentioned bispecific antibodies or the antigen-binding fragments thereof, the second antigen binding domain that specifically binds to the second epitope of human cMET could be changed.

In one embodiment, the second antigen binding domain that specifically binds to the second epitope of human cMET comprises: a heavy chain variable region that comprises (a) a HCDR1 of SEQ ID NO: 61, (b) a HCDR2 of SEQ ID NO: 62, (c) a HCDR3 of SEQ ID NO: 63 and a light chain variable region that comprises: (d) a LCDR1 of SEQ ID NO: 66, (e) a LCDR2 of SEQ ID NO: 67, and (f) a LCDR3 of SEQ ID NO: 68.

In one embodiment, the second antigen binding domain that specifically binds to the second epitope of human cMET comprises
a heavy chain variable region (VH) comprising an amino acid sequence of SEQ ID NO: 59, or SEQ ID NO: 83, and a light chain variable region (VL) comprising an amino acid sequence of SEQ ID NO: 64.

In another embodiment, the bispecific antibody further comprises an amino acid linker described herein. In some embodiments, the amino acid linker has an amino acid sequence comprising any one of SEQ ID NO: 97-139. In some embodiments, the amino acid linker is any sequence of SEQ ID NO: 97-139.

In some embodiments, the antibody drug conjugate comprises a bispecific antibody or a first and second antigen binding domain binding to a first and a second epitope of human cMET, and further comprises a third antigen binding domain that specifically binds to a human tumor associated antigen (TAA) such as human EGFR, as discussed below.

### 3.2 Anti-cMET×Antigen Multispecific Antibodies

In one embodiment, the anti-cMET antibodies disclosed herein (including Section I “anti-cMET Antibodies” and Section 3.1 “anti-cMET Bispecific Antibodies) can be used to construct multispecific antibodies with other modalities such as a human tumor associated antigen (TAA), immune checkpoints or immune stimulators.

In one embodiment, the anti-cMET antibodies as disclosed herein can be incorporated into an anti-cMET×TAA multispecific antibody, wherein anti-TAA is an antibody or fragment thereof directed to any human tumor associated antigen (TAA) other than cMET. An antibody molecule is a multispecific antibody molecule, for example, it comprises at least two antigen binding domains, wherein at least one antigen binding domain sequence specifically binds cMET as a first epitope and a second antigen binding domain sequence specifically binds a TAA as a second epitope.

In another embodiment, two antigen binding domain sequences specifically binds two distinct epitopes (e.g., non-overlapping or non-competing) of human cMET as a first epitope and a second epitope, respectively, and the third antigen binding domain sequence specifically binds a TAA as a third epitope. In one embodiment, the multispecific antibody comprises a third, fourth or fifth antigen binding domain. In one embodiment, the multispecific antibody is a bispecific antibody, a trispecific antibody, or tetraspecific antibody.

In one embodiment, the multispecific antibody is a bispecific antibody. As used herein, a bispecific antibody specifically binds only two antigens. The bispecific antibody comprises a first antigen binding domain which specifically binds human cMET and a second antigen binding domain that specifically binds a TAA. This includes a bispecific antibody comprising a second heavy chain variable domain and a second light chain variable domain which specifically binds a TAA and a first heavy chain variable domain and a first light chain variable domain which specifically binds human cMET. In some embodiments, the bispecific antibody comprises antigen binding fragments, wherein the antigen-binding fragment can be a Fab, F(ab′)₂, Fv, a single chain Fv (scFv), or a single domain antibody.

In one embodiment, the multispecific antibody is a trispecific antibody. As used herein, a trispecific antibody specifically binds at least three antigens or epitopes. The trispecific antibody comprises a first antigen binding domain which specifically binds a first epitope of human cMET, a second antigen binding domain which specifically binds a second epitope of human cMET, and a third antigen binding domain that specifically binds a TAA. This includes a trispecific antibody comprising a third heavy chain variable domain and a third light chain variable domain which specifically binds a TAA, a first heavy chain variable domain and a first light chain variable domain which specifically binds the first epitope of human cMET, and a second heavy chain variable domain and a second light chain variable domain which specifically binds the second epitope of human cMET. In some embodiments, the trispecific antibody comprises antigen binding fragments, wherein the antigen-binding fragment can be a Fab, F(ab′)₂, Fv, a single chain Fv (scFv), or a single domain antibody.

In one embodiment, the multispecific antibody of the present disclosure binds to a human TAA and/or human cMET with a binding affinity (K_{D}) of from 1×10⁻⁶ M to 1×10⁻¹⁰ M, or even 1×10⁻¹¹ M. In another embodiment, the multispecific antibody of the present disclosure binds to a human TAA and at least one epitope on human cMET with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

Previous experimentation (Coloma and Morrison Nature Biotech. 15: 159-163 (1997), incorporated by reference in its entirety) described a tetravalent bispecific antibody which was engineered by fusing DNA encoding a single chain anti-dansyl antibody Fv (scFv) after the C terminus (CH3-scFv) or after the hinge (hinge-scFv) of an lgG3 anti-dansyl antibody. The present disclosure provides multivalent antibodies (e.g., tetravalent antibodies) with at least two antigen binding domains, which can be readily produced by recombinant expression of nucleic acid encoding the polypeptide chains of the antibody. The multivalent antibody herein comprises three to eight, but preferably three or four antigen binding domains, which specifically bind at least two antigens.

In one embodiment, the multispecific antibody is a bispecific antibody, trispecific antibody or tetraspecific antibody. In another embodiment, the multispecific antibody further comprises an amino acid linker described herein. In some embodiments, the amino acid linker has an amino acid sequence comprising any one of SEQ ID NO: 97-139. In some embodiments, the amino acid linker is any sequence of SEQ ID NO: 97-139.

### 3.3 EGFR×cMET Multispecific Antibodies

In one aspect, the present disclosure provides an antibody drug conjugate comprising a multispecific antibody or antigen-binding fragment thereof, comprising a first antigen binding domain that specifically binds a first epitope of human cMET; a second antigen binding domain that specifically binds to a second epitope of human cMET; and a third antigen binding domain that specifically binds to human EGFR.

In one embodiment, the first epitope is distinct from the second epitope, or the first antigen binding domain does not compete with the second antigen binding domain.

In one aspect, EGFR×cMET multispecific antibodies further comprises a third antigen binding domain that specifically binds to human EGFR on the top of anti-cMET bispecific antibodies described in Section 3.1

In one aspect, in the anti-cMET×Antigen multispecific antibodies described in Section 3.2, the further antigen (TAA) is human EGFR.

In one embodiment, the first antigen binding domain that specifically binds a first epitope of human cMET is selected from any anti-cMET antibody described in Section 1.1.

In one embodiment, the second antigen binding domain that specifically binds a second epitope of human cMET is selected from any anti-cMET antibody described in Section 1.2.

In one embodiment, the third antigen binding domain that specifically binds to human EGFR is selected from any anti-EGFR antibody described in Section II.

In one embodiment, in the EGFR×cMET multispecific antibody or antigen-binding fragment thereof, the first antigen binding domain that specifically binds to the first epitope of human cMET comprises: a heavy chain variable region that comprises (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68; the second antigen binding domain that specifically binds to the second epitope of human cMET comprises: a heavy chain variable region that comprises (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and the third antigen binding domain that specifically binds to human EGFR comprises: a heavy chain variable region that comprises (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

In one embodiment, in the EGFR×cMET multispecific antibody or antigen-binding fragment thereof, the first antigen binding domain that specifically binds to the first epitope of human cMET comprises: a heavy chain variable region (VH) comprising an amino acid sequence of SEQ ID NO: 144, and a light chain variable region (VL) comprising an amino acid sequence of SEQ ID NO: 64; the second antigen binding domain that specifically binds to the second epitope of human cMET comprises: a heavy chain variable region (VH) comprising an amino acid sequence of SEQ ID NO: 145, and a light chain variable region (VL) comprising an amino acid sequence of SEQ ID NO: 64; and the third antigen binding domain that specifically binds to human EGFR comprises: a heavy chain variable region (VH) comprising an amino acid sequence of SEQ ID NO: 142, and a light chain variable region (VL) comprising an amino acid sequence of SEQ ID NO: 143.

In one embodiment, the first antigen binding domain that specifically binds to the first epitope of human cMET is a monoclonal antibody, a human engineered antibody, a single chain antibody (scFv), a Fab fragment, a Fab′ fragment, or a F(ab′)2 fragment; the second antigen binding domain that specifically binds to the second epitope of human cMET is a monoclonal antibody, a human engineered antibody, a single chain antibody (scFv), a Fab fragment, a Fab′ fragment, or a F(ab′)2 fragment; and the third antigen binding domain that specifically binds to human EGFR is a monoclonal antibody, a human engineered antibody, a single chain antibody (scFv), a Fab fragment, a Fab′ fragment, or a F(ab′)2 fragment.

In one embodiment, the EGFR×cMET multispecific antibody or antigen-binding fragment thereof comprises a heavy chain constant region of the subclass of IgG1, IgG2, IgG3, or IgG4, and/or a light chain constant region of the type of kappa or lambda.

In one embodiment, the EGFR×cMET multispecific antibody or antigen-binding fragment thereof comprises a heavy chain constant region of the subclass of IgG1, and a light chain constant region of the type of kappa. For example, in some embodiments, the first and second antigen binding domains that specifically bind to epitopes of human cMET form a bispecific antibody, such as an IgG antibody (e.g., a human IgG1, IgG2, IgG3, or IgG4).

In one embodiment, the EGFR×cMET multispecific antibody or antigen-binding fragment thereof has antibody dependent cellular cytotoxicity (ADCC), antibody-dependent cellular phagocytosis (ADCP) or complement dependent cytotoxicity (CDC).

In one embodiment, the EGFR×cMET multispecific antibody or antigen-binding fragment thereof has reduced glycosylation or no glycosylation or is hypofucosylated. In another embodiment, the multispecific antibody or antigen-binding fragment thereof is afucosylated.

In one embodiment, the EGFR×cMET multispecific antibody or antigen-binding fragment thereof comprises increased bisecting GlcNac structures.

In one embodiment, the EGFR×cMET multispecific antibody or antigen-binding fragment thereof comprises a Fc domain, and wherein the Fc domain is an IgG1 Fc. In some embodiments, the IgG1Fc comprises mutations in the heavy chain constant region that may allow for extended half-life after administration in vivo. In some embodiments, the Fc domain comprises one or two heavy chain constant regions with YTE substitutions (M252Y, S254T and T256E).

In one embodiment, the EGFR×cMET multispecific antibody or antigen-binding fragment thereof adopts knob into hole (KIH) to form heterodimer. For example, in some embodiments, the above IgG1Fc comprises one heavy chain with a knob modification such as T366W and a second heavy chain with a hole modification such as T366S, L368A, and Y407V.

In one embodiment, the first antigen binding domain that specifically binds to the first epitope of human cMET comprises a first heavy chain constant region comprising SEQ ID NO: 95, and the second antigen binding domain that specifically binds to the second epitope of human cMET comprises a second heavy chain constant region comprising SEQ ID NO: 96.

In another embodiment, the first antigen binding domain that specifically binds to the first epitope of human cMET comprises a first heavy chain constant region comprising SEQ ID NO: 96, and the second antigen binding domain that specifically binds to the second epitope of human cMET comprises a second heavy chain constant region comprising SEQ ID NO: 95.

In another embodiment, the first antigen binding domain that specifically binds to the first epitope of human cMET and the second antigen binding domain that specifically binds to the second epitope of human cMET share a common light chain, i.e., comprising the same amino acid sequence.

In one embodiment, the first light chain constant region and the second light chain constant region are different. In another embodiment, the first light chain constant region and the second light chain constant region are the same.

In one embodiment, the light chain constant region has an amino acid sequence of SEQ ID NO: 71. In one embodiment, the light chain constant region is SEQ ID NO: 71.

In one embodiment, the first light chain variable region and the second light chain variable region are different. In another embodiment, the first light chain variable region and the second light chain variable region are the same.

In one embodiment, the EGFR×cMET multispecific antibody or antigen-binding fragment thereof further comprises amino acid linker herein.

In some embodiments, the third antigen binding domain that specifically binds to human EGFR is an antigen binding fragment, such as an scFv.

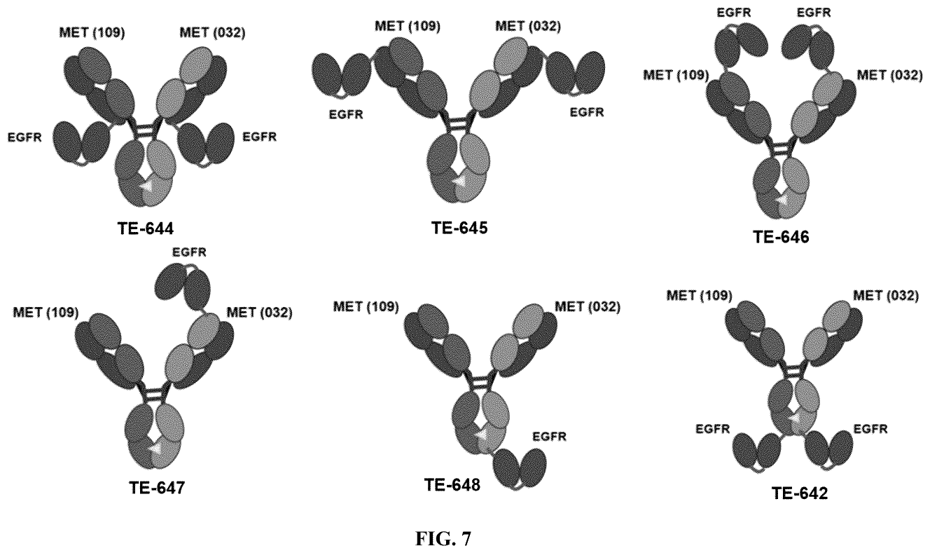

In one embodiment, the EGFR×cMET multispecific antibody or antigen-binding fragment thereof could be in different formats with different EGFR scFv valency and orientation to cMET bispecific antibody arms. The format could be any format shown in FIG. 7, represented by TE-644, TE-645, TE-646, TE-647, TE-648 or TE-642. For example, in one embodiment, the first antigen binding domain that specifically binds to the first epitope of human cMET and the second antigen binding domain that specifically binds to the second epitope of human cMET comprise a bispecific IgG antibody, and the third antigen binding domain that specifically binds to human EGFR comprises an scFv with an orientation as follows, as shown in FIG. 7: (a) the scFv is attached to the C-terminal of a light chain of one or both of the first and second antigen binding domains; (b) the scFv is attached to the N-terminal of a light chain of one or both of the first and second antigen binding domains; (c) the scFv is attached to the N-terminal of a heavy chain of both of the first and second antigen binding domains; (d) the scFv is attached to the N-terminal of a heavy chain of one of the first and second antigen binding domains; (e) the scFv is attached to the C-terminal of a heavy chain constant region of one of the first and second antigen binding domains; or (f) the scFv is attached to the C-terminal of a heavy chain constant region of both of the first and second antigen binding domains.

In one embodiment, the EGFR×cMET multispecific antibody or antigen-binding fragment thereof comprises a first polypeptide, a second polypeptide, a third polypeptide, and a fourth polypeptide, and optionally at least one amino acid linker, wherein (1) a VL of the third antigen binding domain that specifically binds to human EGFR, optionally a first amino acid linker, a VH of the third antigen binding domain that specifically binds to human EGFR, optionally a second amino acid linker, a VH of the second antigen binding domain that specifically binds to the second epitope of human cMET, and the second heavy chain constant region are arranged in the first polypeptide in the direction of N terminal to C terminal; (2) a VH of the first antigen binding domain that specifically binds to the first epitope of human cMET, and the first heavy chain constant region are arranged in the second polypeptide in the direction of N terminal to C terminal; or a VL of the third antigen binding domain that specifically binds to human EGFR, optionally a first amino acid linker, a VH of the third antigen binding domain that specifically binds to human EGFR, optionally a second amino acid linker, a VH of the first antigen binding domain that specifically binds to the first epitope of human cMET, the first heavy chain constant region, are arranged in the second polypeptide in the direction of N terminal to C terminal; (3) a VL of the second antigen binding domain that specifically binds to the second epitope of human cMET, a second light chain constant region, are arranged in the third polypeptide in the direction of N terminal to C terminal; and (4) a VL of the first antigen binding domain that specifically binds to the first epitope of human cMET, a first light chain constant region, are arranged in the fourth polypeptide in the direction of N terminal to C terminal.

In one embodiment, the EGFR×cMET multispecific antibody or antigen-binding fragment thereof comprises a first polypeptide, a second polypeptide, a third polypeptide, and a fourth polypeptide, and optionally at least one amino acid linker, wherein (1) a VL of the third antigen binding domain that specifically binds to human EGFR, optionally a first amino acid linker, a VH of the third antigen binding domain that specifically binds to human EGFR, optionally a second amino acid linker, a VH of the second antigen binding domain that specifically binds to the second epitope of human cMET, and a second heavy chain constant region are arranged in the first polypeptide in the direction of N terminal to C terminal; (2) a VH of the first antigen binding domain that specifically binds to the first epitope of human cMET, and a first heavy chain constant region are arranged in the second polypeptide in the direction of N terminal to C terminal; (3) a VL of the second antigen binding domain that specifically binds to the second epitope of human cMET, a second light chain constant region, are arranged in the third polypeptide in the direction of N terminal to C terminal; and (4) a VL of the first antigen binding domain that specifically binds to the first epitope of human cMET, a first light chain constant region, are arranged in the fourth polypeptide in the direction of N terminal to C terminal.

In one embodiment, the second heavy chain constant region is SEQ ID NO: 95, and the first heavy chain constant region is SEQ ID NO: 96; or the second heavy chain constant region is SEQ ID NO: 96, and the first heavy chain constant region is SEQ ID NO: 95.

In one embodiment, the first amino acid linker is SEQ ID NO: 139. In another embodiment, the second amino acid linker is SEQ ID NO: 139.

In one embodiment, a VL of the first antigen binding domain and a VL of the second antigen binding domain are the same.

In another embodiment, the first light chain constant region and the second light chain constant region are the same. In another embodiment, the first light chain constant region and the second light chain constant region are SEQ ID NO: 71.

In one embodiment, the third polypeptide and the fourth polypeptide are the same.

In another embodiment, the position of the first antigen binding domain that specifically binds to the first epitope of human cMET (such as BGA-109) and the second antigen binding domain that specifically binds to the second epitope of human cMET (such as BGA-032) could be exchanged.

In one embodiment, the multispecific antibody or antigen-binding fragment thereof comprises a first polypeptide, a second polypeptide, and a third polypeptide, wherein
(1) the first polypeptide has an amino acid sequence of SEQ ID NO: 153, the second polypeptide has an amino acid sequence of SEQ ID NO: 150, and the third polypeptide has an amino acid sequence of SEQ ID NO: 148;
(2) the first polypeptide has an amino acid sequence of SEQ ID NO: 146, the second polypeptide has an amino acid sequence of SEQ ID NO: 147, and the third polypeptide has an amino acid sequence of SEQ ID NO: 148;
(3) the first polypeptide has an amino acid sequence of SEQ ID NO: 149, the second polypeptide has an amino acid sequence of SEQ ID NO: 150, and the third polypeptide has an amino acid sequence of SEQ ID NO: 151;
(4) the first polypeptide has an amino acid sequence of SEQ ID NO: 149, the second polypeptide has an amino acid sequence of SEQ ID NO: 150, and the third polypeptide has an amino acid sequence of SEQ ID NO: 152;
(5) the first polypeptide has an amino acid sequence of SEQ ID NO: 153, the second polypeptide has an amino acid sequence of SEQ ID NO: 154, and the third polypeptide has an amino acid sequence of SEQ ID NO: 148;
(6) the first polypeptide has an amino acid sequence of SEQ ID NO: 146, the second polypeptide has an amino acid sequence of SEQ ID NO: 150, and the third polypeptide has an amino acid sequence of SEQ ID NO: 148;
(7) the first polypeptide has an amino acid sequence of SEQ ID NO: 168, the second polypeptide has an amino acid sequence of SEQ ID NO: 169, and the third polypeptide has an amino acid sequence of SEQ ID NO: 148;
(8) the first polypeptide has an amino acid sequence of SEQ ID NO: 153, the second polypeptide has an amino acid sequence of SEQ ID NO: 170, and the third polypeptide has an amino acid sequence of SEQ ID NO: 148;
(9) the first polypeptide has an amino acid sequence of SEQ ID NO: 168, the second polypeptide has an amino acid sequence of SEQ ID NO: 171, and the third polypeptide has an amino acid sequence of SEQ ID NO: 148; or
(10) the first polypeptide has an amino acid sequence of SEQ ID NO: 153, the second polypeptide has an amino acid sequence of SEQ ID NO: 172, and the third polypeptide has an amino acid sequence of SEQ ID NO: 148.

In another embodiment, the multispecific antibody or antigen-binding fragment thereof of comprises
(1) a first polypeptide of SEQ ID NO: 153, the second polypeptide of SEQ ID NO: 150, and the third polypeptide of SEQ ID NO: 148;
(2) the first polypeptide of SEQ ID NO: 146, the second polypeptide of SEQ ID NO: 147, and the third polypeptide of SEQ ID NO: 148;
(3) the first polypeptide of SEQ ID NO: 149, the second polypeptide of SEQ ID NO: 150, and the third polypeptide of SEQ ID NO: 151;
(4) the first polypeptide of SEQ ID NO: 149, the second polypeptide of SEQ ID NO: 150, and the third polypeptide of SEQ ID NO: 152;
(5) the first polypeptide of SEQ ID NO: 153, the second polypeptide of SEQ ID NO: 154, and the third polypeptide of SEQ ID NO: 148;
(6) the first polypeptide of SEQ ID NO: 146, the second polypeptide of SEQ ID NO: 150, and the third polypeptide of SEQ ID NO: 148;
(7) the first polypeptide has an amino acid sequence of SEQ ID NO: 168, the second polypeptide has an amino acid sequence of SEQ ID NO: 169, and the third polypeptide has an amino acid sequence of SEQ ID NO: 148;
(8) the first polypeptide has an amino acid sequence of SEQ ID NO: 153, the second polypeptide has an amino acid sequence of SEQ ID NO: 170, and the third polypeptide has an amino acid sequence of SEQ ID NO: 148;
(9) the first polypeptide has an amino acid sequence of SEQ ID NO: 168, the second polypeptide has an amino acid sequence of SEQ ID NO: 171, and the third polypeptide has an amino acid sequence of SEQ ID NO: 148; or
(10) the first polypeptide has an amino acid sequence of SEQ ID NO: 153, the second polypeptide has an amino acid sequence of SEQ ID NO: 172, and the third polypeptide has an amino acid sequence of SEQ ID NO: 148.

### IV. Additional Antibody Modifications

### Constant Region and Fc Region

In some embodiments, an antibody drug conjugate herein may comprise an antibody component comprising a heavy chain constant region and/or a light chain constant region. In some cases, the antibody is a multispecific antibody in IgG format, such as comprising human IgG heavy and light chains. In some cases, the antibody comprises a bispecific IgG antibody comprising human IgG heavy chain constant regions, optionally that interact to form an Fc domain, for example.

The heavy chain constant region could be a wild type sequence of heavy chain constant regions from the human subclass of IgG1, IgG2, IgG3, or IgG4. The light chain constant region could be a wild type sequence of light chain from the human kappa or lambda type. In one embodiment, the heavy chain constant region comprises a wild type sequence of constant region from human IgG1. In some cases, the human IgG heavy chain constant region sequence comprises a C-terminal lysine or C-terminal glycine lysine, while in other cases it does not. The light chain constant region in come cases may comprise a wild type sequence of light chain from human kappa chain. In one embodiment, the heavy chain constant region comprises the amino acid sequences of SEQ ID NO: 70, with or without a C-terminal lysine or C-terminal glycine-lysine, and the light chain constant region comprises the amino acid sequence of SEQ ID NO: 71. In one embodiment, the Fc region could be wild type Fc region of the subclass of human IgG1, IgG2, IgG3, or IgG4.

In one embodiment, the antibody or antigen-binding fragment thereof comprises a Fc domain of IgG1 or IgG4 with reduced effector function. In another embodiment, the heavy chain constant region comprises mutations E233P, L234A, L235A, G236del, and P329A.

In one embodiment, the antibody or antigen-binding fragment thereof comprised within the antibody drug conjugate comprises an Fc domain with extended half-life after administration in vivo compared to a wild-type Fc domain. In another embodiment, the antibody or antigen-binding fragment thereof comprises an Fc domain of human IgG1, comprising the “YTE” mutation (M252Y/S254T/T256E, EU numbering, as described in U.S. Pat. No. 7,658,921, incorporated by reference in its entirety), which is located at CH2 of the IgG Fc region. In some cases the YTE mutation may be found on both heavy chain constant region sequences of the Fc domain, or in other cases on one of the heavy chain constant region sequences.

In another embodiment, antibodies of the present disclosure have strong Fc-mediated effector functions, and the antibodies mediate antibody-dependent cellular cytotoxicity (ADCC) against target cells.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al, each incorporated by reference in their entirety.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al, incorporated by reference in its entirety.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009), incorporated by reference in its entirety.

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001), incorporated by reference in its entirety.

In still another aspect, the glycosylation of the antibody is modified. For example, an aglycosylated antibody can be made (i.e., the antibody lacks or has reduced glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al, incorporated by reference in its entirety.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., incorporated by reference in its entirety, describes a cell line with a functionally disrupted FUT8 gene, which encodes a fucosyl transferase, such that antibodies expressed in such a cell line exhibit hypofucosylation. Publication WO 03/035835, incorporated by reference in its entirety, by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740), incorporated by reference in its entirety. WO99/54342 by Umana et al., incorporated by reference in its entirety, describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999, incorporated by reference in its entirety).

WO2003085107A1 by Naoko Ohnuki et al., describes an engineered CHO cell line in which the activity of α1, 6-fucosyltransferase is lower or disappeared, so that the produced antibodies or antigen-binding fragment thereof is afucosylated.

In another aspect, if a reduction of ADCC is desired, human antibody subclass IgG4 was shown in many previous reports to have only modest ADCC and almost no CDC effector function (Moore G L, et al., 2010 MAbs, 2:181-189, incorporated by reference in its entirety). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19, each incorporated by reference in their entirety). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157, incorporated by reference in its entirety). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19, each incorporated by reference in their entirety). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50, each incorporated by reference in their entirety). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19, each incorporated by reference in their entirety). To generate multispecific antibodies with low ADCC and CDC but with good stability, it is possible to modify the hinge and Fc region of human IgG4 and introduce a number of alterations. These modified IgG4 Fc molecules can be found in SEQ ID NOs: 83-88 of U.S. Pat. No. 8,735,553 to Li et al, each incorporated by reference in their entirety.

In another embodiment, the antibody of the present disclosure comprises Fc domain of human IgG4 with S228P and/or R409K substitutions (according to EU numbering system).

“Knob-into-hole” mutations could also be incorporated into the Fc: Fc binding interfaces. In some embodiments, knob-into-holes insure the correct pairing of two different heavy chains together during the manufacture of multispecific antibodies. In one embodiment, the first heavy chain constant region or the first Fc of the multispecific antibodies herein comprises SEQ ID NO: 95, and the second heavy chain constant region or the second Fc of the multispecific antibodies herein comprises SEQ ID NO: 96. In another embodiment, the first heavy chain constant region or the first Fc of the multispecific antibodies herein comprises SEQ ID NO: 96, and the second heavy chain constant region or the second Fc of the multispecific antibodies herein comprises SEQ ID NO: 95.

In another embodiment, the first heavy chain constant region or the first Fc of the multispecific antibodies herein comprises a knob variant of human IgG1 constant region comprising T366W, and second heavy chain constant region or the second Fc of the multispecific antibodies herein comprises a hole variant of human IgG1 constant region comprising T366S, L368A, and Y407V (EU Numbering).

In another embodiment, the first heavy chain constant region or the first Fc of the multispecific antibodies herein comprises variant of human IgG1 constant region comprising T366S, L368A, and Y407V, and second heavy chain constant region or the second Fc of the multispecific antibodies herein comprises variant of human IgG1 constant region comprising T366W (EU Numbering).

In some cases, more than one type of mutation may be found in an Fc domain, such as a combination of YTE mutations and also knob-into-hole mutations such as T366S, L368A, and Y407V or T366W.

### Amino Acid Linkers

It is also understood that the domains and/or regions of the polypeptide chains of the conjugate can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1. Such linker region may comprise a random assortment of amino acids, or a restricted set of amino acids. Such linker regions can be flexible or rigid (see e.g., US2009/0155275, incorporated by reference in its entirety).

Multispecific antibodies have been constructed by genetically fusing two single chain Fv (scFv) or Fab fragments with or without the use of flexible linkers (Mallender et al., J. Biol. Chem. 1994 269:199-206; Mack et al., Proc. Natl. Acad. Sci. USA. 1995 92:7021-5; Zapata et al., Protein Eng. 1995 8.1057-62), via a dimerization device such as leucine Zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et al., Biochemistry 1992.31:1579-84; Pack et al., Bio/Technology 1993 11:1271-7). Each reference mentioned in this paragraph incorporated by reference in their entirety.

The antibodies or proteins as disclosed herein comprise an amino acid linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine are comprised within the linker region. In another embodiment, the linker can be GS (SEQ ID NO: 97), GGS (SEQ ID NO: 98), GSG (SEQ ID NO: 99), SGG (SEQ ID NO: 100), GGG (SEQ ID NO: 101), GGGS (SEQ ID NO: 102), SGGG (SEQ ID NO: 103), GGGGS (SEQ ID NO: 104), GGGGSGS (SEQ ID NO: 105), GGGGSGS (SEQ ID NO: 106), GGGGSGGS (SEQ ID NO: 107), GGGGSGGGGS (SEQ ID NO: 108), GGGGSGGGGSGGGGS (SEQ ID NO: 109), AKTTPKLEEGEFSEAR (SEQ ID NO: 110), AKTTPKLEEGEFSEARV (SEQ ID NO: 111), AKTTPKLGG (SEQ ID NO: 112), SAKTTPKLGG (SEQ ID NO: 113), AKTTPKLEEGEFSEARV (SEQ ID NO: 114), SAKTTP (SEQ ID NO: 115), SAKTTPKLGG (SEQ ID NO: 116), RADAAP (SEQ ID NO: 117), RADAAPTVS (SEQ ID NO: 118), RADAAAAGGPGS (SEQ ID NO: 119), RADAAAA(G₄S)₄ (SEQ ID NO: 120), SAKTTP (SEQ ID NO: 121), SAKTTPKLGG (SEQ ID NO: 122), SAKTTPKLEEGEFSEARV (SEQ ID NO: 123), ADAAP (SEQ ID NO: 124), ADAAPTVSIFPP (SEQ ID NO: 125), TVAAP (SEQ ID NO: 126), TVAAPSVFIFPP (SEQ ID NO: 127), QPKAAP (SEQ ID NO: 128), QPKAAPSVTLFPP (SEQ ID NO: 129), AKTTPP (SEQ ID NO: 130), AKTTPPSVTPLAP (SEQ ID NO: 131), AKTTAP (SEQ ID NO: 132), AKTTAPSVYPLAP (SEQ ID NO: 133), ASTKGP (SEQ ID NO: 134), ASTKGPSVFPLAP (SEQ ID NO: 135), GENKVEYAPALMALS (SEQ ID NO: 136), GPAKELTPLKEAKVS (SEQ ID NO: 137), and GHEAAAVMQVQYPAS (SEQ ID NO: 138), GGGGSGGGGSGGGGSGGGGS (SEQ ID NO: 139) or any combination thereof (see WO2007/024715).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551, each incorporated by reference in their entirety. The dimerization specific amino acids can also be within the Fc domain. Also, the dimerization specific amino acids within the Fc domain can be in combination with dimerization specific amino acids within the CH1 or CL domains. In one embodiment, the disclosure provides a multispecific antibody comprising at least one dimerization specific amino acid pair.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies or proteins described herein, e.g., polynucleotides encoding heavy chain variable regions or light chain variable regions comprising the complementarity determining regions as described herein. In some aspects, the polynucleotide encoding the heavy chain variable regions has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide selected from SEQ ID NO: 5, SEQ ID NO: 162, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, SEQ ID NO: 55, SEQ ID NO: 163, or SEQ ID NO: 60. In some aspects, the polynucleotide encoding the light chain variable regions has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide selected from SEQ ID NO: 65.

The present disclosure also provides polynucleotides encoding the scFv described herein.

The polynucleotides of the present disclosure can encode the variable region sequence of the multispecific antibody described herein. They can also encode both a variable region and a constant region of the multispecific antibody. In another embodiment, the polynucleotides of the present disclosure can encode the amino acid sequences of fusion proteins described herein.

In some embodiments, the polynucleotides described herein could be codon-optimized for expression in host cells, e.g., eukaryotic cells, more specifically mammalian cells (e.g., CHO cells).

In some embodiments, the present disclosure provides polynucleotides encoding polypeptides of the multispecific antibodies herein, e.g., EGFR×cMET multispecific antibodies described herein.

In one embodiment, the polynucleotide encoding the first polypeptide of EGFR×cMET multispecific antibody has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide having SEQ ID NO: 164, the polynucleotide encoding the second polypeptide of EGFR×cMET multispecific antibody has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide having SEQ ID NO: 165, and the polynucleotide encoding the third polypeptide of EGFR×cMET multispecific antibody has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide having SEQ ID NO: 166.

In one embodiment, the polynucleotide encoding the first polypeptide of EGFR×cMET multispecific antibody has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide of SEQ ID NO: 164, the polynucleotide encoding the second polypeptide of EGFR×cMET multispecific antibody has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide of SEQ ID NO: 165, and the polynucleotide encoding the third polypeptide of EGFR×cMET multispecific antibody has at least 85%, at least 89%, at least 90%, at least 91%, at least 92%, at least 93%, at least 94%, at least 95%, at least 96%, at least 97%, at least 98%, at least 99%, or 100% nucleic acid sequence identity with a polynucleotide of SEQ ID NO: 166.

In one embodiment, the first polynucleotide encoding the first polypeptide of EGFR×cMET multispecific antibody comprises a DNA sequence having SEQ ID NO: 164, the second polynucleotide encoding the second polypeptide of EGFR×cMET multispecific antibody comprises a DNA sequence having SEQ ID NO: 165, and the third polynucleotide encoding the third polypeptide of EGFR×cMET multispecific antibody comprises a DNA sequence having SEQ ID NO: 166.

In one embodiment, the first polynucleotide encoding the first polypeptide of EGFR×cMET multispecific antibody comprises a DNA sequence of SEQ ID NO: 164, the second polynucleotide encoding the second polypeptide of EGFR×cMET multispecific antibody comprises a DNA sequence of SEQ ID NO: 165, and the third polynucleotide encoding the third polypeptide of EGFR×cMET multispecific antibody comprises a DNA sequence of SEQ ID NO: 166.

In one embodiment, a multispecific antibody herein is constructed from one or more polynucleotides of SEQ ID NOs: 175-179, which encode an anti-EGFR VH, an anti-EGFR VL, the heavy chain of a second antigen binding domain that specifically binds to a second epitope of human cMET, the heavy chain of a first antigen binding domain that specifically binds to a first epitope of human cMET, and the light chain of the first and second antigen binding domains, respectively. In another embodiment, a multispecific antibody herein is constructed from one or more polynucleotides of SEQ ID NOs: 175, 176, 177, 180, and 179, which encode an anti-EGFR VH, an anti-EGFR VL, the heavy chain of a second antigen binding domain that specifically binds to a second epitope of human cMET, the heavy chain of a first antigen binding domain that specifically binds to a first epitope of human cMET, and the light chain of the first and second antigen binding domains, respectively. In yet another embodiment, a multispecific antibody herein is constructed from one or more polynucleotides of SEQ ID NOs: 175, 176, 177, 181, and 179, which encode an anti-EGFR VH, an anti-EGFR VL, the heavy chain of a second antigen binding domain that specifically binds to a second epitope of human cMET, the heavy chain of a first antigen binding domain that specifically binds to a first epitope of human cMET, and the light chain of the first and second antigen binding domains, respectively.

In some embodiments, the polynucleotides described herein could be codon-optimized for expression in host cells, e.g., eukaryotic cells, more specifically mammalian cells (e.g., CHO cells).

In one embodiment, the fucosyltransferase inhibitor 2F-Peracetyl-Fucose (Sigma-Aldrich) for inhibition of fucosylation is added in the production system for effector function enhancement.

In one embodiment, an engineered CHO cell line is used, in which the activity of α1, 6-fucosyltransferase is lower or disappeared, so that the produced antibodies or antigen-binding fragment thereof is afucosylated. Refer to WO2003085107A1 for more details.

Also provided in the present disclosure are expression vectors and host cells for producing the antibodies herein. The choice of expression vector depends on the intended host cells in which the vector is to be expressed. Typically, the expression vectors contain a promoter and other regulatory sequences (e.g., enhancers) that are operably linked to the polynucleotides encoding antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987), each incorporated by reference in their entirety. For example, the SV40 enhancer or CMV enhancer can be used to increase expression in mammalian host cells.

The host cells for harboring and expressing the antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies. Insect cells in combination with baculovirus vectors can also be used. In other aspects, mammalian host cells are used to express and produce the antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987, each incorporated by reference in their entirety. Expression vectors for mammalian host cells can include expression control sequences, such as an origin of replication, a promoter, and an enhancer (see, e.g., Queen et al., Immunol. Rev. 89:49-68, 1986, incorporated by reference in its entirety), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### V. Antibody Drug Conjugates, Part I

The antibodies disclosed herein may be combined with a payload (P, PA, or D herein), such as a cytotoxic agent, to form an antibody drug conjugate (ADC). The cytotoxic agent may be any molecule that inhibits or reduces the expression of molecules in cells, inhibits or reduces the function of cells, induces apoptosis of cells, and/or causes death of cells. Examples of cytotoxic agents include those described herein. In embodiments, the cytotoxic agent is a topoisomerase inhibitor.

In embodiments, an antibody drug conjugate has the Formula A:

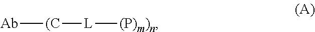

or a pharmaceutically acceptable salt, solvate, or hydrate thereof, wherein:
Ab is an antibody or antigen-binding fragment thereof disclosed herein;
C is a conjugator;
L is a linker;
P is a payload;
m is an integer from 1 to 8; and
n is from 1 to 15.

In particular embodiments, m is 1.

In embodiments, an ADC has the Formula A-1:

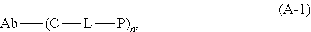

or a pharmaceutically acceptable salt, solvate, or hydrate thereof, wherein:
Ab is an antibody or antigen-binding fragment thereof disclosed herein;
C is a conjugator;
L is a linker;
P is a payload; and
n is from 1 to 15.

In embodiments, n is from 3 to 15, e.g., from 3 to 12, from 4 to 11, or from 5 to 10. In some embodiments n is from 7 to 9. In some embodiments, n is about 8. In some embodiments n is from 5 to 7. In some embodiments, n is about 6.

In embodiments, an ADC has Formula (I):

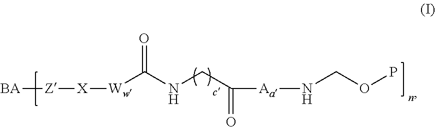

or a pharmaceutically acceptable salt, tautomer, solvate, or stereoisomer thereof,
   wherein:
BA is Ab, as that variable is described with respect to an antibody drug conjugate of the present disclosure (e.g., an antibody drug conjugate of Formula A or A-1);
Z′ is

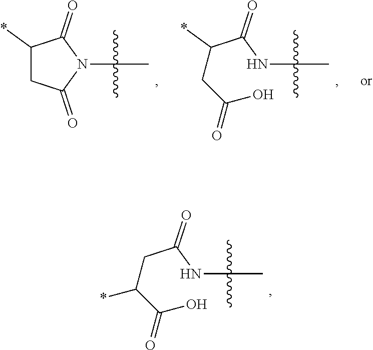 [SMILES: *C1CC(=O)N(*)C1=O ; *NC(=O)C(*)CC(=O)O ; *NC(=O)CC(*)C(=O)O]

and * indicates the bond where Z′ attaches to BA (e.g., Ab);
X is a substituted or unsubstituted C₁₋₅ alkyl;
W is oxygen;
w′ is 0 or 1;
c′ is 1 or 2;
a′ is 1, 2, or 3;
each A is, independently, an amino acid residue optionally substituted with a hydrophilic group;
P is a payload residue; and
n is from 1 to 15.

Alternative values for variables BA (Ab) are as disclosed herein, for example, with respect to Anti-cMET Antigen Binding Domains, Anti-EGFR Antigen Binding Domains, Anti-cMET Bispecific Antibodies, Anti-cMET×Antigen Multispecific Antibodies, and EGFR×cMET Multispecific Antibodies.

In some embodiments, X is an C₃ alkyl. In other embodiments, X is a C₅ alkyl. In some embodiments, X is —(CH₂)₃—, —(CH₂)₅—, —CH(CH₂NH₂)(CH₂)₂—, —CH(CH₂NH₂)(CH₂)₄—, —CH₂CH(NH₂)CH₂—, —CH(CH₂NH₂)CH₂—, or —CH(CH₂NH₂)(CH₂)₂—. In some embodiments, X is —(CH₂)₅— or —CH(CH₂NH₂)(CH₂)₂—.

In some embodiments, w′ is 0. In other embodiments, w′ is 1.

In some embodiments, c′ is 1. In other embodiments, c′ is 2.

In some embodiments, a′ is 1. In other embodiments, a′ is 2. In other embodiments, a′ is 3.

In embodiments, each A is, independently, a residue of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine. In some embodiments, each A is, independently, a residue of glycine, phenylalanine, serine, or valine.

Each A may be substituted with a hydrophilic group (HG). In some embodiments, the hydrophilic group is a saccharide, phosphate ester, sulfate ester, a phosphodiester, or a phosphonate. In further embodiments, the hydrophilic group is a saccharide, and the saccharide is β-D-galactose, N-acetyl-β-D-galactosamine, N-acetyl-a-D-galactosamine, N-acetyl-β-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or an O-acetyl modification thereof.

In some embodiments the hydrophilic group (HG) is

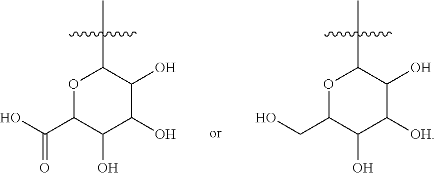 [SMILES: *C1OC(C(=O)O)C(O)C(O)C1O ; "or" ; *C1OC(CO)C(O)C(O)C1O]

In some embodiments, the hydrophilic group (HG) is

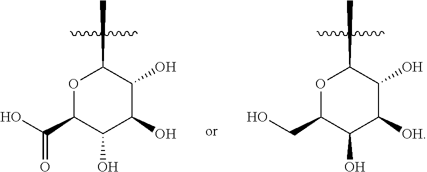

In one embodiment the hydrophilic group (HG) is

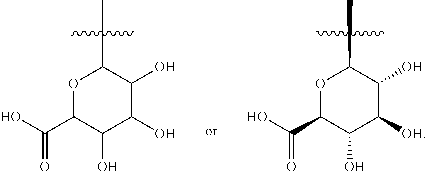

Alternative values for HG are as described herein with respect to HG¹ as described in Section VI, Antibody Drug Conjugates and Linker-Payloads, Part II.

In some embodiments, A is

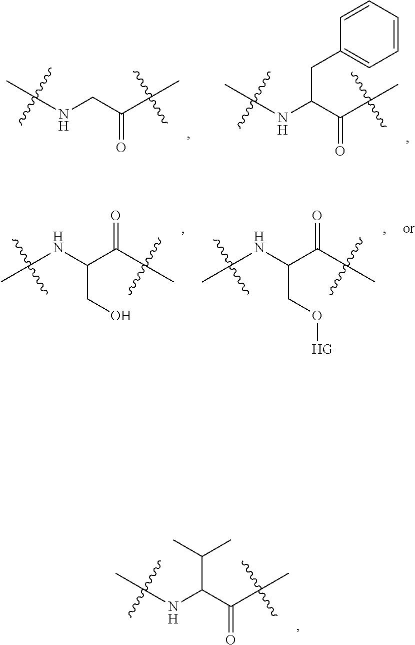

wherein HG is the hydrophilic group. In some embodiments, A is

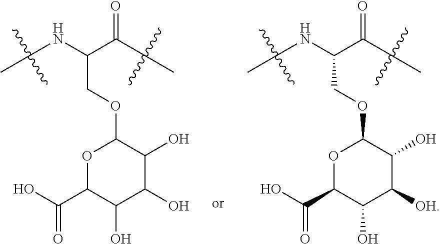 [SMILES: *NC(COC1OC(C(=O)O)C(O)C(O)C1O)C(*)=O ; "or" ; *N[C@@H](CO[C@@H]1O[C@H](C(=O)O)[C@@H](O)[C@H](O)[C@H]1O)C(*)=O]

In some embodiments, A is:

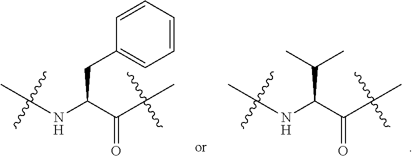

In some embodiments, Aₐ′ is

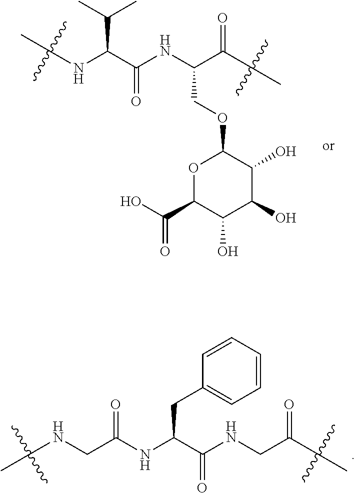 [SMILES: *N[C@H](C(=O)N[C@@H](CO[C@@H]1O[C@H](C(=O)O)[C@@H](O)[C@H](O)[C@H]1O)C(*)=O)C(C)C ; *NCC(=O)N[C@@H](Cc1ccccc1)C(=O)NCC(*)=O]

Payload residue, P, may be a residue of an exatecan analogue.

In some embodiments, P is:

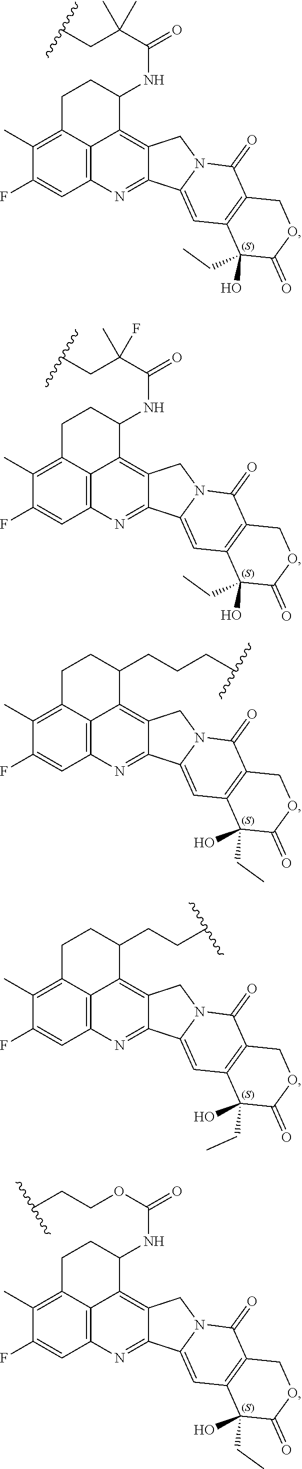

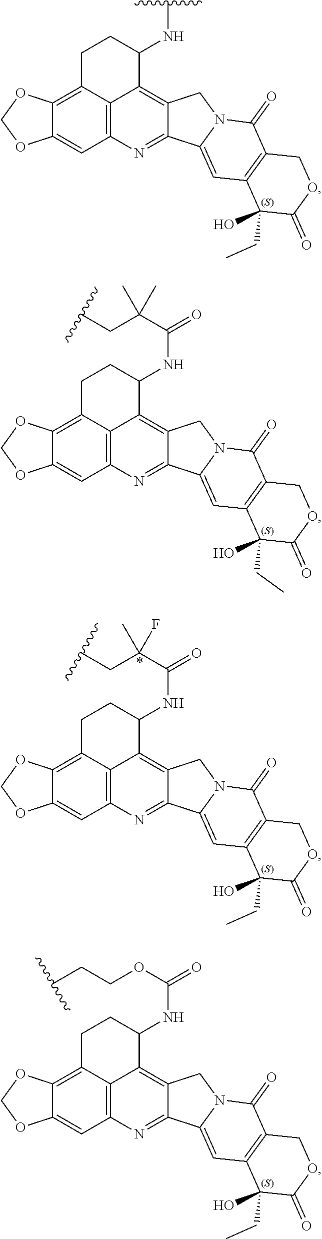

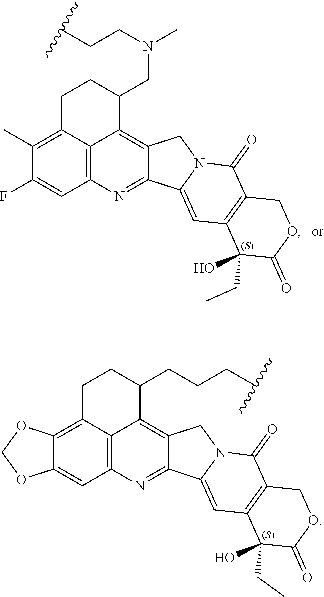

In one embodiment, P is

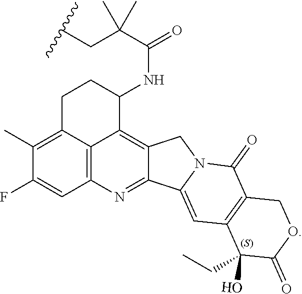

In some embodiments, n is from 3 to 15, e.g., from 3 to 12, from 4 to 11, or from 5 to 10. In some embodiments n is from 7 to 9. In some embodiments, n is about 8. In some embodiments n is from 5 to 7. In some embodiments, n is about 6.

In some embodiments, an ADC is represented by one of the following formulas, or a pharmaceutically acceptable salt, solvate, and/or stereoisomer thereof:

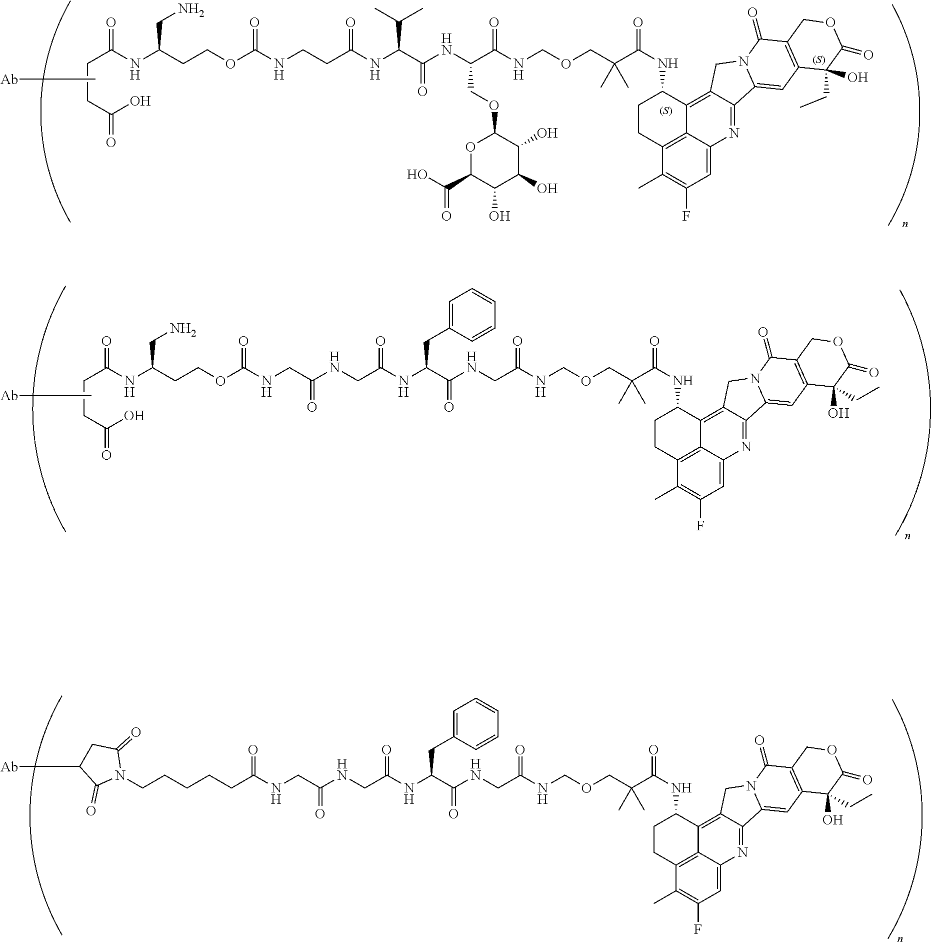

wherein Ab is any antibody or antigen-binding fragment thereof disclosed herein, for example, with respect to Anti-cMET Antigen Binding Domains, Anti-EGFR Antigen Binding Domains, Anti-cMET Bispecific Antibodies, Anti-cMET×Antigen Multispecific Antibodies, and EGFR×cMET Multispecific Antibodies, and n is from 1 to 15.

In some embodiments, an ADC is represented by one of the following formulas, or a pharmaceutically acceptable salt, solvate, and/or stereoisomer thereof:

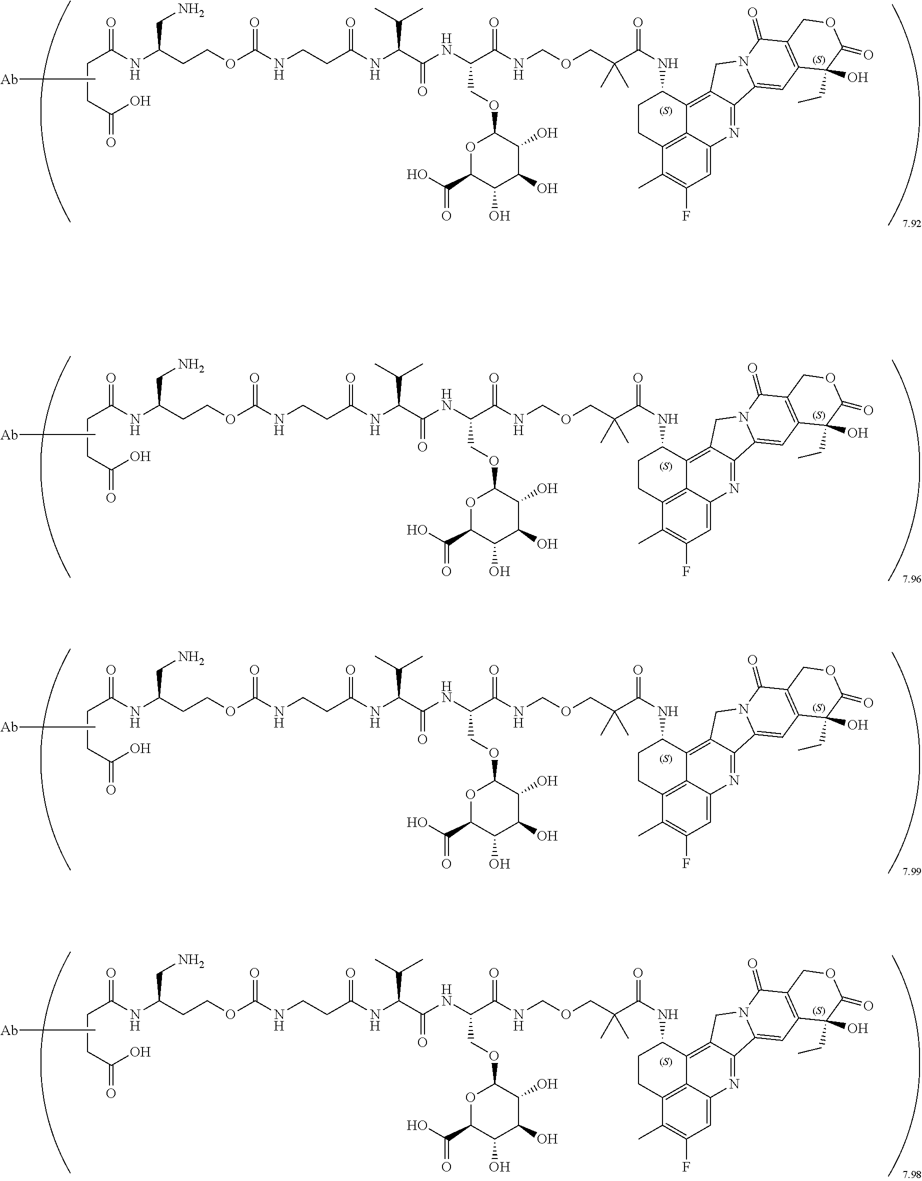

wherein Ab is any antibody or antigen-binding fragment thereof disclosed herein, for example, with respect to Anti-cMET Antigen Binding Domains, Anti-EGFR Antigen Binding Domains, Anti-cMET Bispecific Antibodies, Anti-cMET×Antigen Multispecific Antibodies, and EGFR×cMET Multispecific Antibodies.

In some embodiments, an ADC is represented by one of the following formulas, or a pharmaceutically acceptable salt, solvate, and/or stereoisomer thereof:

| ADC | Antibody | |
|---|---|---|
| No. | (Ab) | ADC structure |
| ADC-1 | BG-T187 FE BGX2 | 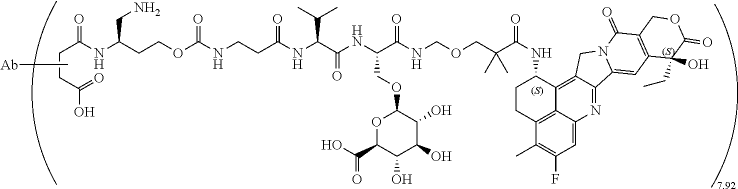 |
| ADC-2 | BG-T187 HE BGX3 | 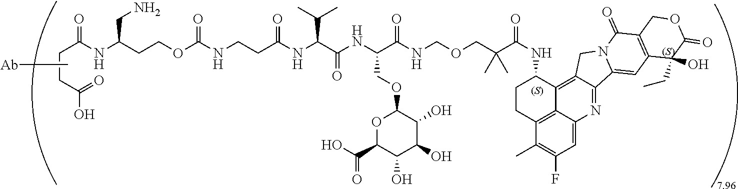 |
| ADC-3 | BG-T187 wt BGX1 | 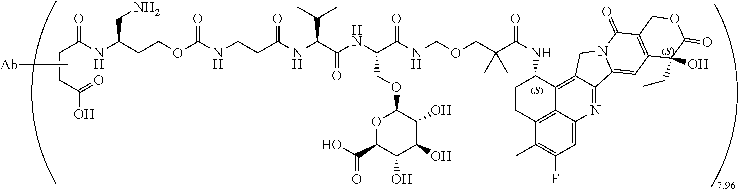 |
| ADC-4 | BG-T187 NE + YTE BGX5 | 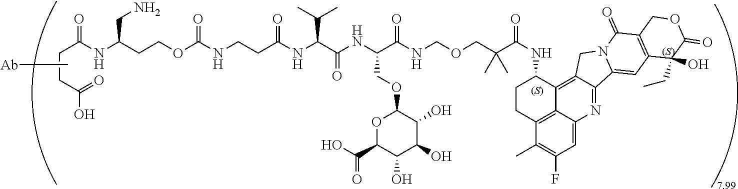 |
| ADC-5 | BG-T187 FE + TYE BGX4 | 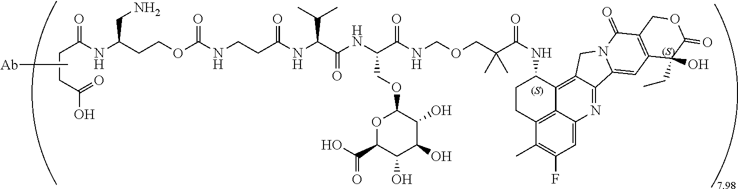 |
| ADC-6 | BG-T187 FE + YTE BGX4 | 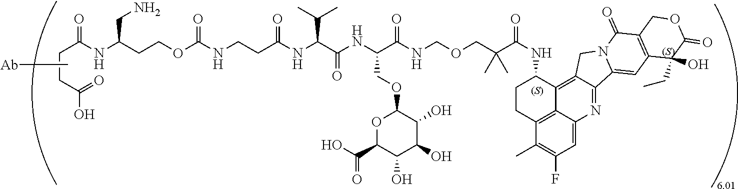 |

### VI. Linker-Payloads, Part I

Also provided herein are covalent linkers, e.g., for use in an ADC described herein.

In some embodiments the linker is a compound of Formula (II):

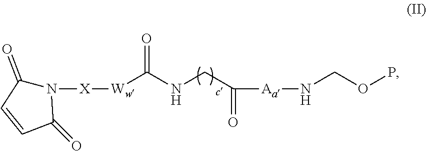

or a pharmaceutically acceptable salt, tautomer, solvate, or stereoisomer thereof, wherein:
X is a substituted or unsubstituted C₁₋₅ alkyl;
W is oxygen;
subscript w′ is 0 or 1;
c′ is 1 or 2;
a′ is 1, 2, or 3;
each A is, independently, an amino acid residue optionally substituted with a hydrophilic group; and
P is a payload residue.

Alternative values for the variables (e.g., X, W, w′, c′, A, a′, P) are as described herein. For example, alternative values for variables X, W, w′, c′, A, a′, and P are as described herein with respect to Section IV, Antibody Drug Conjugates.

### VII. Antibody Drug Conjugates and Linker-Payloads, Part II

PCT Publication No. WO 2023/125530, the entire contents of which are incorporated herein by reference, discloses antibody drug conjugates, the linker payload portions of which are suitable for use in the context of the present disclosure, and linker payloads which are suitable for use in the context of the present disclosure. In some embodiments, a linker payload is a linker payload disclosed in WO 2023/125530.

In embodiments, an antibody drug conjugate has Formula (I*):

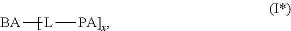

or a pharmaceutically acceptable salt, tautomer, solvate, stereoisomer, enantiomer, isotopologue, or prodrug thereof,
wherein BA is Ab, as that variable is described with respect to an antibody drug conjugate of the present disclosure (e.g., an antibody drug conjugate of Formula A or A-1); L is a covalent linker; PA is a payload residue (e.g., a cytotoxic agent (D), as that variable is described with respect to an antibody drug conjugate of the present disclosure (e.g., an antibody drug conjugate of Formula A or A-1); and subscript x is from 1 to 30 (e.g., n as that variable is described with respect to an antibody drug conjugate of the present disclosure (e.g., an antibody drug conjugate of Formula A or A-1). In some instances, subscript x is from 1 to 4. In some instances, subscript x is about 1. In some instances, subscript x is about 2. In some instances, subscript x is about 3. In some instances, subscript x is about 4.

In further embodiments, the antibody drug conjugate has Formula (Ia*):

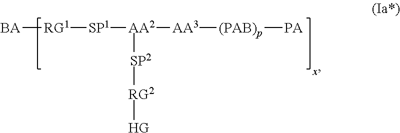

or a pharmaceutically acceptable salt tautomer, solvate, stereoisomer, enantiomer, isotopologue, or prodrug thereof,
wherein RG¹ is a reactive group residue; RG² is an optional reactive group residue; SP¹ and SP² are independently, in each instance, an optional spacer group residue; HG¹ is a hydrophilic residue; PAB is an optional self-immolative unit; subscript p is 0 or 1; and subscript x is from 1 to 30. Values for the remaining variables (e.g., AA², AA³) and alternative values for the variables (e.g., x, p, PAB, HG¹, RG¹, RG², SP¹, SP², BA) are as described elsewhere herein.

In some embodiments, subscript x is from 1 to 15. In some embodiments, subscript x is from 2 to 10. In some embodiments, subscript x is from 3 to 9. In one embodiment, subscript x is about 3. In one embodiment, subscript x is about 4. In one embodiment, subscript x is about 5. In one embodiment, subscript x is about 6. In one embodiment, subscript x is about 7. In one embodiment, subscript x is about 8. In one embodiment, subscript x is about 9.

In some embodiments of a compound of Formula (Ia*), AA² comprises formula (W*):

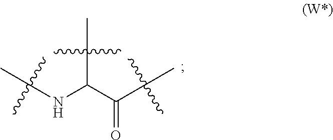 [SMILES: *NC(*)C(*)=O]

AA³ is a dipeptide residue of -valine-alanine-, -valine-citrulline-, or

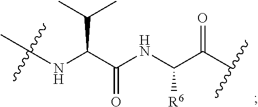

wherein R⁶ is —CH₃, or —(CH₂)₃—NHC(═O)NH₂.

In some embodiments,

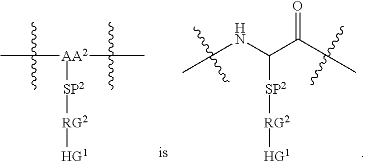

In some embodiments, AA³ is

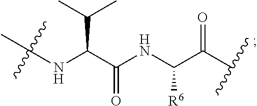

wherein R⁶ is —CH₃, or —(CH₂)₃—NHC(═O)NH₂. In further embodiments, R⁶ is —CH₃.

In some embodiments, PAB represents —NH—CH2-O—, formula (Y1*):

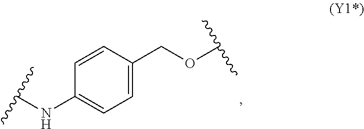 [SMILES: *Nc1ccc(CO*)cc1]

or formula (Y2*):

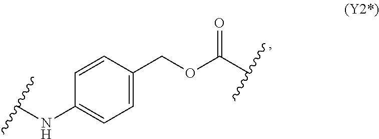 [SMILES: *Nc1ccc(COC(*)=O)cc1]

wherein the

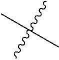

indicates the bond through which the PAB is bonded to the adjacent groups in the formula.

In some embodiments, PAB is —NH—CH₂—O—.

In some embodiments, RG¹ is

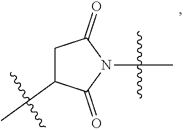 [SMILES: *C1CC(=O)N(*)C1=O]

-(succinimide-3-yl-N)—,

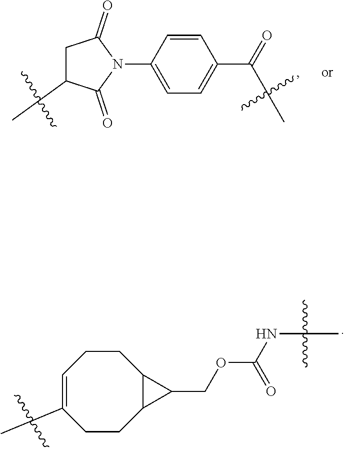 [SMILES: *C(=O)c1ccc(N2C(=O)CC(*)C2=O)cc1 ; *NC(=O)OCC1C2CC/C=C(/*)CCC21]

In some embodiments, RG¹ is

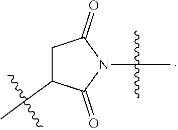 [SMILES: *C1CC(=O)N(*)C1=O]

In some embodiments, RG¹ is

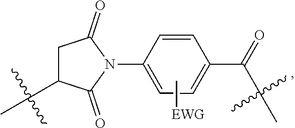

wherein EWG is an electrowithdrawing group, e.g., —CN, —NO₂, halogen, —CF₃, —C(═O)OR¹, or —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In some embodiments, RG¹ is

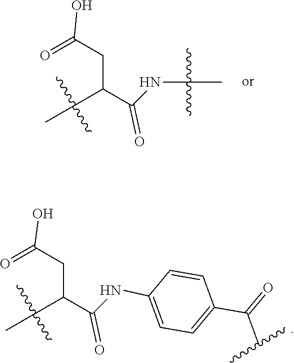 [SMILES: *NC(=O)C(*)CC(=O)O ; *C(=O)c1ccc(NC(=O)C(*)CC(=O)O)cc1]

In some embodiments, RG¹ is

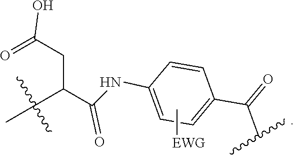

wherein EWG is an electrowithdrawing group, e.g., —CN, —NO₂, halogen, —CF₃, —C(═O)OR¹, or —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In some embodiments, RG¹ is an opened heterocyclic ring, such as the product resulting from conjugation of the maleimide ring of

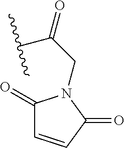 [SMILES: *C(=O)CN1C(=O)C=CC1=O]

with an antibody. It will be appreciated in this regard that conjugation of an antibody to a maleimide ring can occur at either one of the two carbons in the carbon-carbon double bond of the maleimide. Similarly, in the context of an opened ring, conjugation can occur at either one of the two carbon atoms in the double bond. In some embodiments, RG¹ is

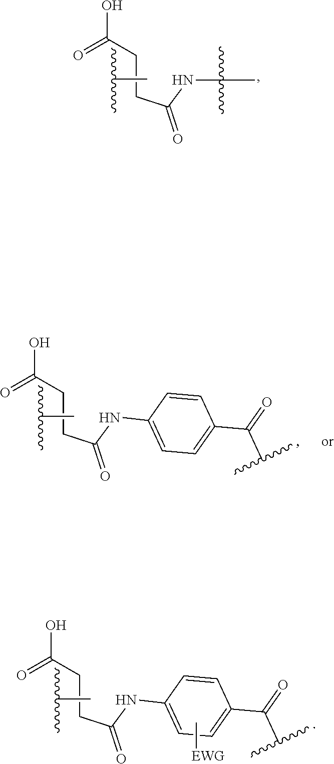

In some embodiments, RG¹ is

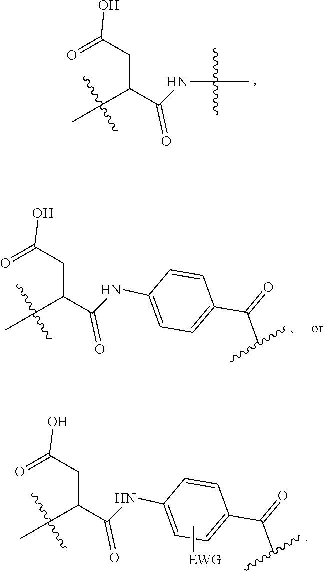

In some embodiments, RG¹ is

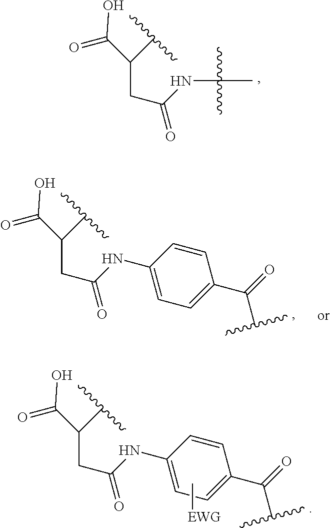

In some embodiments, RG¹ is

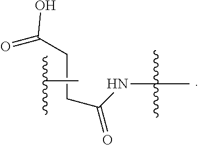

In some embodiments, RG¹ is

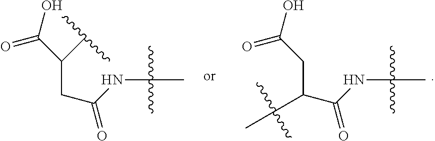 [SMILES: *NC(=O)CC(*)C(=O)O ; *NC(=O)C(*)CC(=O)O]

In some embodiments, RG² is a bond, —C(═O)—NH—, or —NHC(═O)—. In some embodiments, RG² is —C(═O)—NH—.

In some embodiments, SP¹ is —(CH₂)ₙ₁—C(═O)—, —(CH₂CH₂O)ₙ₂—CH₂CH₂—C(═O)—, —CH[—(CH₂)ₙ₃—COOH]—C(═O)—, —CH₂—C(═O)—NH—(CH₂)ₙ₄—C(═O)—, —CH₂—C(═O)—NH—(CH₂)ₙ₃—C(═O)—NH—(CH₂)ₙ₄—C(═O)—, or —C(═O)—(CH₂)ₙ₅—C(═O)—, wherein each of n1, n2, n3, n4, and n5 independently represents an integer of 1 to 8. In some embodiments, SP¹ is *—CH₂C(O)N(H)CH₂CH₂C(O)—, wherein asterisk marks the bond that connects to RG¹. In some embodiments, SP¹ is *—(CH₂)₅C(O)—, wherein asterisk marks the bond that connects to RG¹. In some embodiments, SP¹ is *—C(H)(CH₂NH₂)—(CH₂)₂₀C(O)N(H)(CH₂)₂C(O)—, wherein asterisk marks the bond that connects to RG¹.

In some embodiments, SP² is —(CH₂)ₙ₆—; and n6 represents an integer of 1 to 8. In some embodiments, n6 is 2.

In some embodiments, HG¹ is

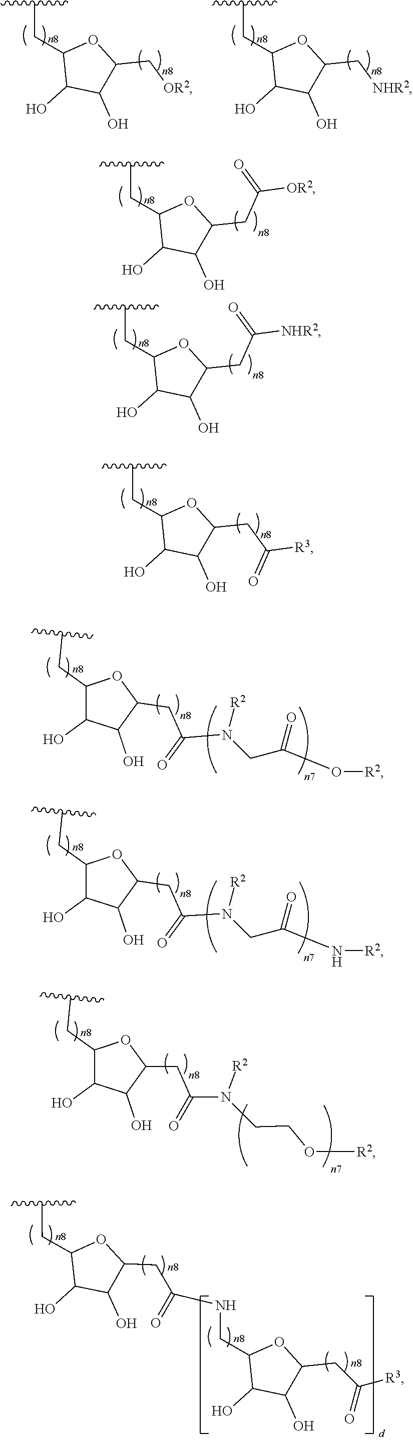

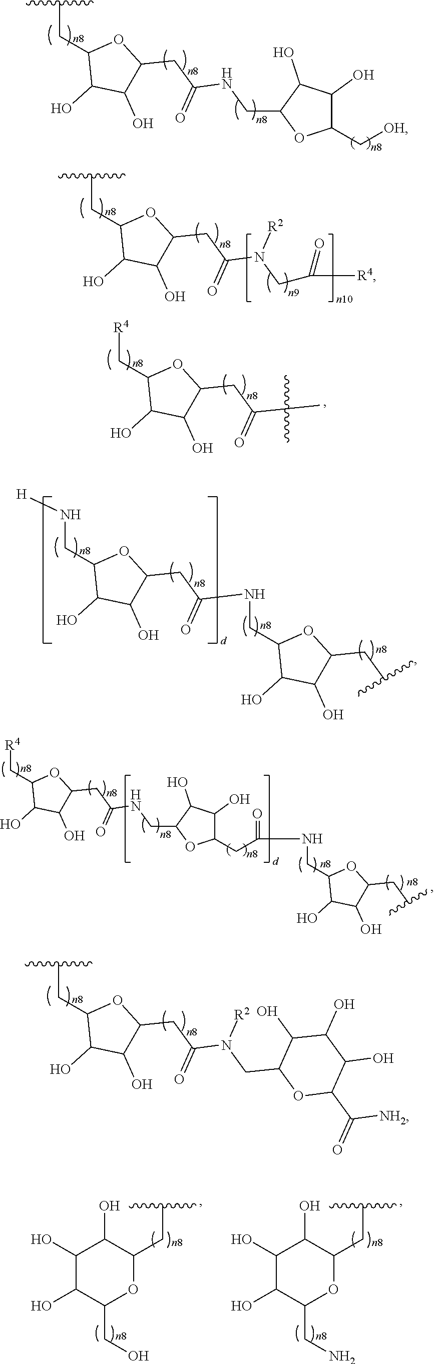

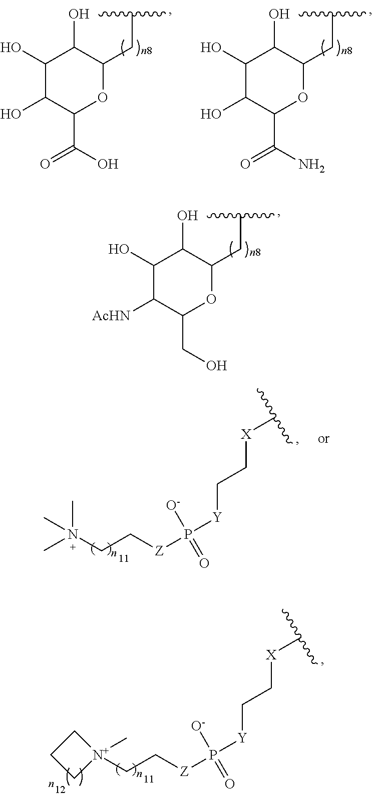

wherein each n7 is independently 1-15; each n8 is independently 0 or 1; each n9 is independently 1 or 2; each n10 is independently an integer of 4 to 16, such as 4, 8, or 12; each n1l is independently an integer of 0 to 5; n12 is an integer of 0 to 3; d is 0-3; R² is H or Me; R³ is —OH, —NH₂, —NHCH₂—CH₂-(PEG)ₓ-OH, or —NHCH₂—CH₂-(PEG)ₓ-OMe; R⁴ is OH or NH₂; and each of X, Y, and Z is independently —CH₂—, —NH—, —S— or —O—.

In some embodiments, HG¹ is

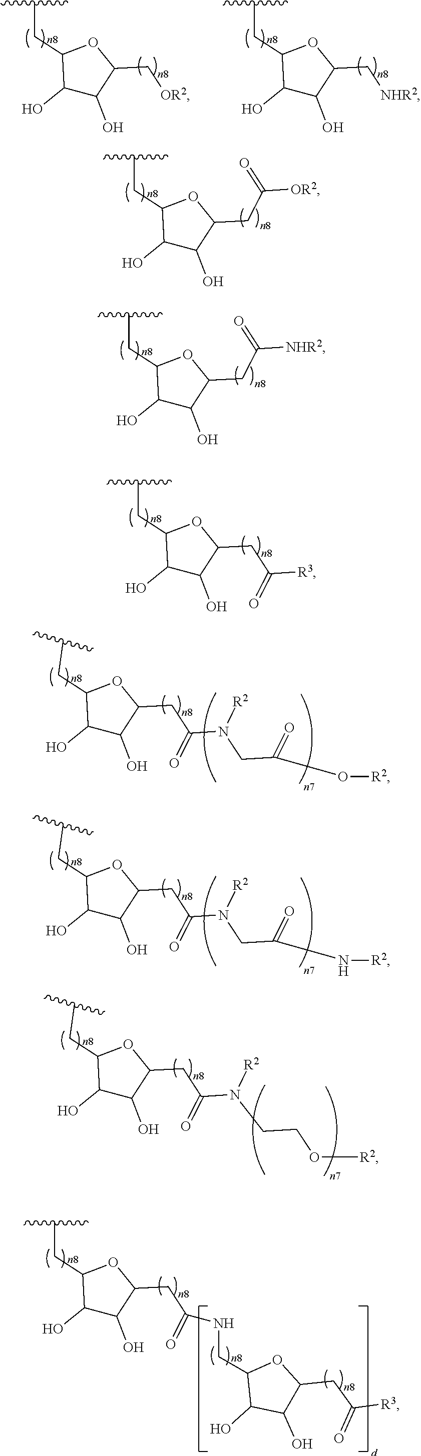

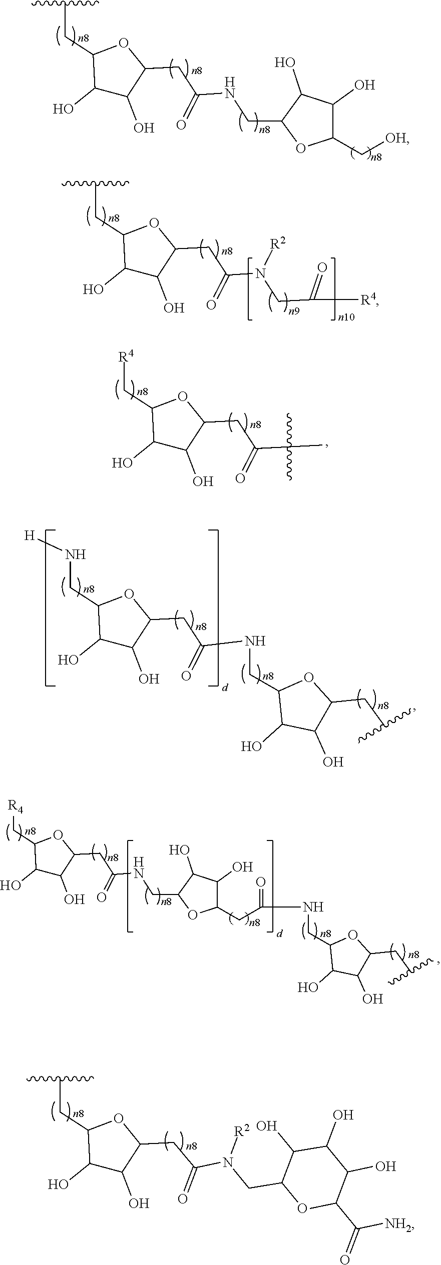

wherein each n7 is independently 1-15; each n8 is independently 0 or 1; each n9 is independently 1 or 2; each n10 is independently an integer of 4 to 16, such as 4, 8, or 12; d is 0-3; R² is H or Me; R³ is —OH, —NH₂, —NHCH₂—CH₂-(PEG)ₓ-OH, or —NHCH₂—CH₂-(PEG)ₓ-OMe; R⁴ is OH or NH₂.

In some embodiments, HG¹ is

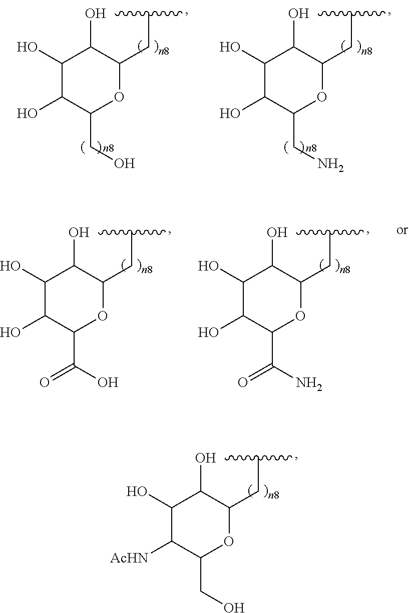

wherein each n8 is independently 0 or 1; and R¹ is H or Me.

In some embodiments, HG¹ is

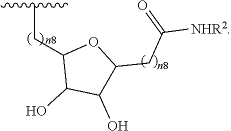

In some embodiments, HG¹ is

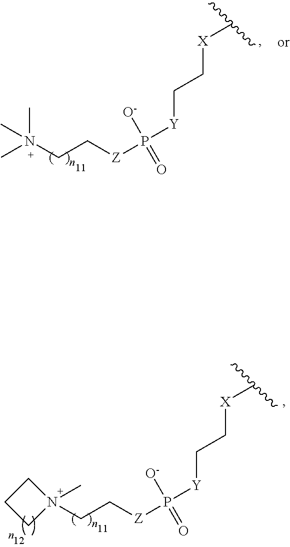

wherein each n11 is independently an integer of 0 to 5; n12 is an integer of 0 to 3; and each of X, Y, and Z is independently —CH₂—, —NH—, —S— or —O—.

In some embodiments, HG¹ is —NHSO₂NH₂, —SO₃H, —SO₂NH₂, —PO₃H₂, and RG² is a bond.

In some embodiments,

AA² is an amino acid residue of glycine or

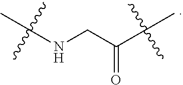 [SMILES: *NCC(*)=O]

In some embodiments of compounds of Formula (Ia*) or a pharmaceutically acceptable salt tautomer, solvate, stereoisomer, enantiomer, isotopologue, or prodrug thereof, AA² comprises formula (W*):

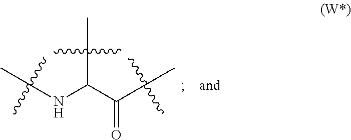 [SMILES: *NC(*)C(*)=O]

AA³ is a tetrapeptide residue of -glycine-glycine-phenylalanine-glycine- or

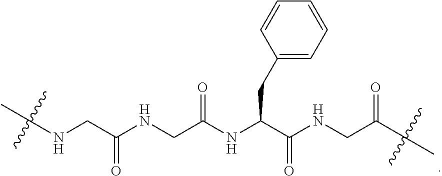 [SMILES: *NCC(=O)NCC(=O)N[C@@H](Cc1ccccc1)C(=O)NCC(*)=O]

In further embodiments, the antibody drug conjugate has Formula (Ib*):

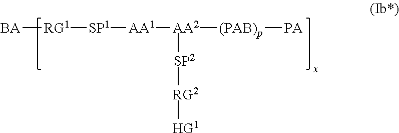

or a pharmaceutically acceptable salt tautomer, solvate, stereoisomer, enantiomer, isotopologue, or prodrug thereof,
wherein AA² comprises formula (W*):

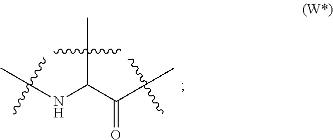 [SMILES: *NC(*)C(*)=O]

and
AA¹ is a dipeptide residue of -valine-alanine-, -valine-citrulline-, or

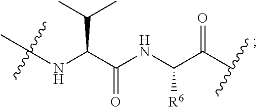

wherein R⁶ is —CH₃, or —(CH₂)₃—NHC(═O)NH₂. Values for the remaining variables (e.g., x, p, BA, HG¹, RG¹, RG², SP¹, SP², PAB, PA) and alternative values for the variables (e.g., AA¹, AA²) are as described elsewhere herein, for example, with respect to compounds of Formula (Ia*).

In some embodiments, AA² comprises formula (W*):

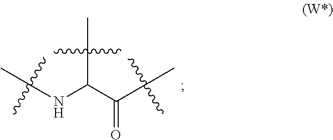 [SMILES: *NC(*)C(*)=O]

and
AA¹ is a tetrapeptide residue of -glycine-glycine-phenylalanine-glycine- or

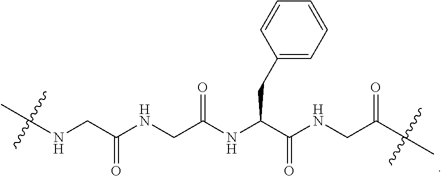 [SMILES: *NCC(=O)NCC(=O)N[C@@H](Cc1ccccc1)C(=O)NCC(*)=O]

In further embodiments, the antibody drug conjugate has Formula (Ic*):

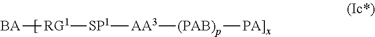

or a pharmaceutically acceptable salt tautomer, solvate, stereoisomer, enantiomer, isotopologue, or prodrug thereof,
wherein AA³ is a dipeptide residue of -valine-alanine-, -valine-citrulline-, or

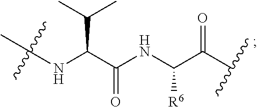

wherein R⁶ is —CH₃, or —(CH₂)₃—NHC(═O)NH₂. Values for the remaining variables (e.g., BA, RG¹, SP¹, PAB, p, PA, x) and alternative values for the variables (AA³, R⁶) are as described elsewhere herein, for example, with respect to compounds of formula (Ia*).

In some embodiments (e.g., of a compound of Formula (Ic*)), AA³ is a tetrapeptide residue of -glycine-glycine-phenylalanine-glycine- or

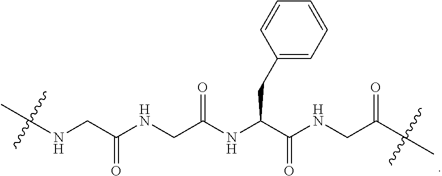 [SMILES: *NCC(=O)NCC(=O)N[C@@H](Cc1ccccc1)C(=O)NCC(*)=O]

In some embodiments, each PA independently represents formula (D1*):

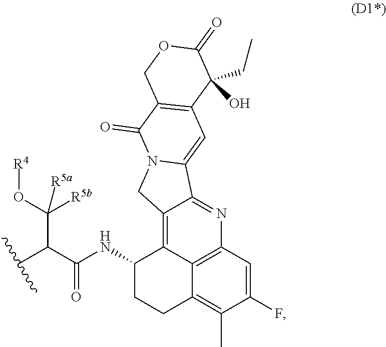

wherein each of R⁴, R^{5a}, and R^{5b} is independently hydrogen, sugar residue, substituted or unsubstituted inorganic or organic acid residue, substituted or unsubstituted C₁₋₈ alkyl, substituted or unsubstituted cycloalkyl, substituted or unsubstituted non-aromatic heterocyclyl, substituted or unsubstituted cycloalkylalkyl, or substituted or unsubstituted heterocyclylalkyl; R^{5a} and R^{5b} together with the atoms to which they are attached, form a substituted or unsubstituted cycloalkyl, substituted or unsubstituted non-aromatic heterocyclyl.

In some embodiments, R⁴ is hydrogen,

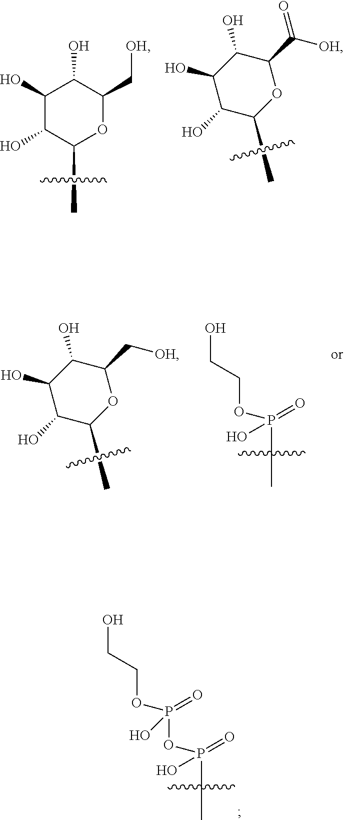

and
wherein each of R^{5a} and R^{5b} is independently H, CH₃, or CF₃; or R^{5a} and R^{5b} together with the atoms to which they are attached, form a substituted or unsubstituted cycloalkyl, substituted or unsubstituted non-aromatic hetercyclyl.

In some embodiments, R⁴ is hydrogen,

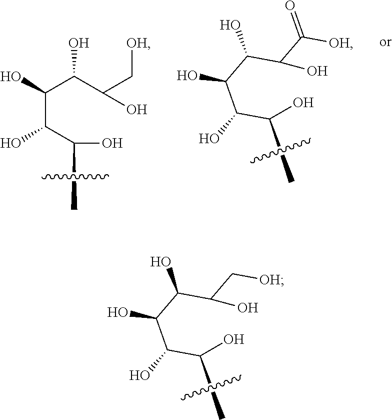

and
wherein each of R^{5a} and R^{5b} is independently H, CH₃, or CF₃; or
R^{5a} and R^{5b} together with the atoms to which they are attached, form a substituted or unsubstituted cycloalkyl, substituted or unsubstituted non-aromatic heterocyclyl.

In some embodiments, each PA independently represents

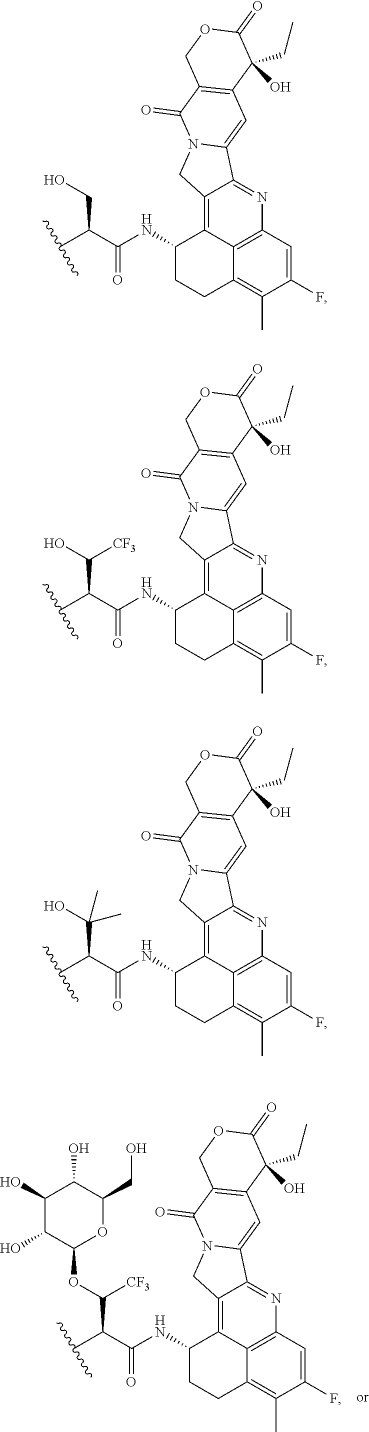

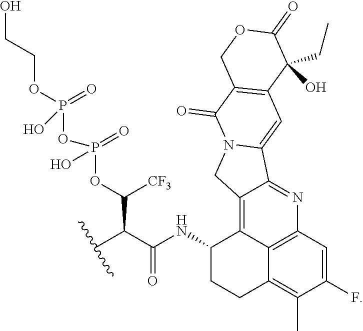 [SMILES: *[C@H](C(=O)N[C@H]1CCc2c(C)c(F)cc3nc4c(c1c23)Cn1c-4cc2c(c1=O)COC(=O)[C@]2(O)CC)C(OP(=O)(O)OP(=O)(O)OCCO)C(F)(F)F]

In some embodiments, each PA independently represents formula (D2*):

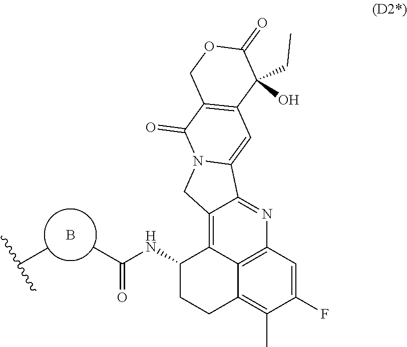

wherein ring B is a substituted or unsubstituted cycloalkyl, substituted or unsubstituted aryl, substituted or unsubstituted heterocyclyl, or substituted or unsubstituted heteroaryl.

In some embodiments, each PA independently represents

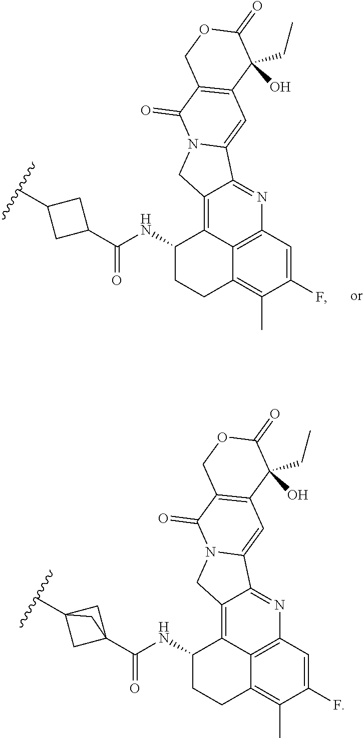 [SMILES: *C1CC(C(=O)N[C@H]2CCc3c(C)c(F)cc4nc5c(c2c34)Cn2c-5cc3c(c2=O)COC(=O)[C@]3(O)CC)C1 ; "or" ; *C12CC(C(=O)N[C@H]3CCc4c(C)c(F)cc5nc6c(c3c45)Cn3c-6cc4c(c3=O)COC(=O)[C@]4(O)CC)(C1)C2]

In some embodiments, each PA independently represents formula (D3*):

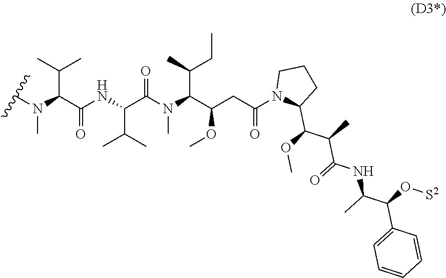

wherein S² is an enzyme hydrolyzable hydrophilic group.

In some embodiments, the S² group is hydrogen or represents one of the following formulas:

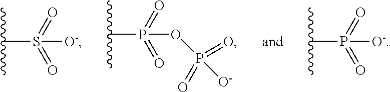

In some embodiments, each PA independently represents formula (E1*):

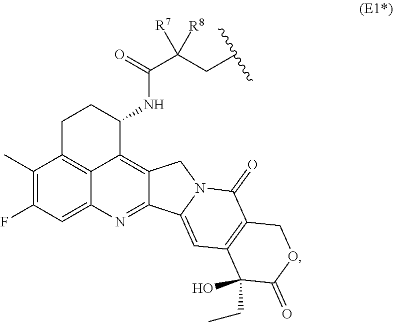

wherein each of R⁷ and R⁸ is, independently, hydrogen, halogen, or alkyl.

In some embodiments, R⁷ and R⁸ are hydrogen.

In some embodiments, R⁷ and R⁸ are methyl.

In some embodiments, R⁷ is methyl and R⁸ is F.

In some embodiments, the carbon to which R⁷ and R⁸ connect is in the S configuration.

In some embodiments, the carbon to which R⁷ and R⁸ connect is in the R configuration.

In some embodiments, each PA independently represents the following formula:

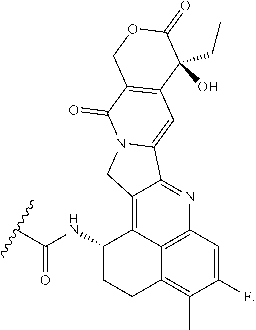 [SMILES: *C(=O)N[C@H]1CCc2c(C)c(F)cc3nc4c(c1c23)Cn1c-4cc2c(c1=O)COC(=O)[C@]2(O)CC]

In some embodiments, each PA is independently Dxd, or independently represents the following formula:

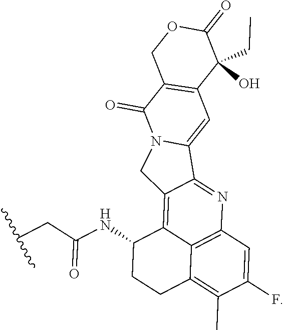 [SMILES: *CC(=O)N[C@H]1CCc2c(C)c(F)cc3nc4c(c1c23)Cn1c-4cc2c(c1=O)COC(=O)[C@]2(O)CC]

In some embodiments, the antibody drug conjugate is selected from one of the following compounds, or a pharmaceutically acceptable salt tautomer, solvate, stereoisomer, enantiomer, isotopologue, or prodrug thereof:

| Example | |
|---|---|
| No. | Antibody Drug Conjugate Structure |
| ADC1.2 | 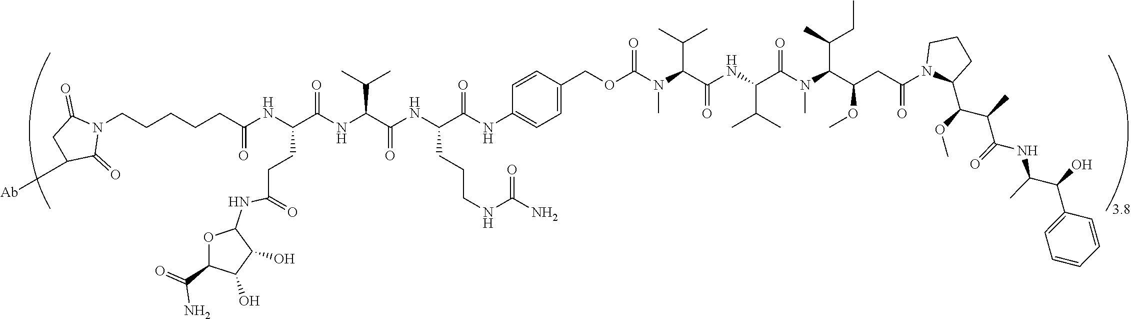 |
| ADC1.4 | 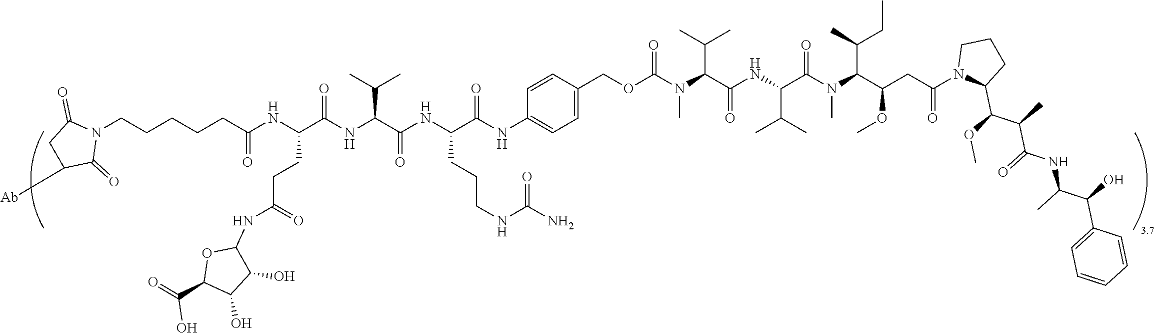 |
| ADC1.11 | 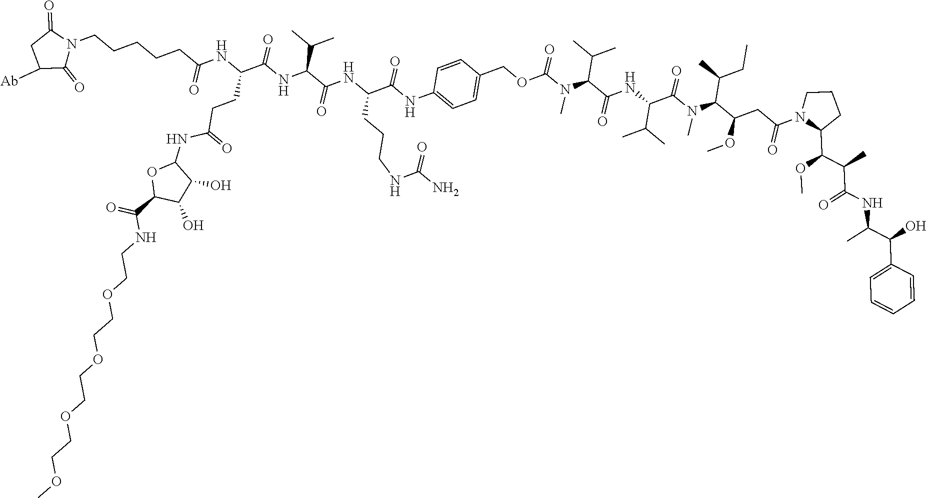 |
| ADC2.2 | 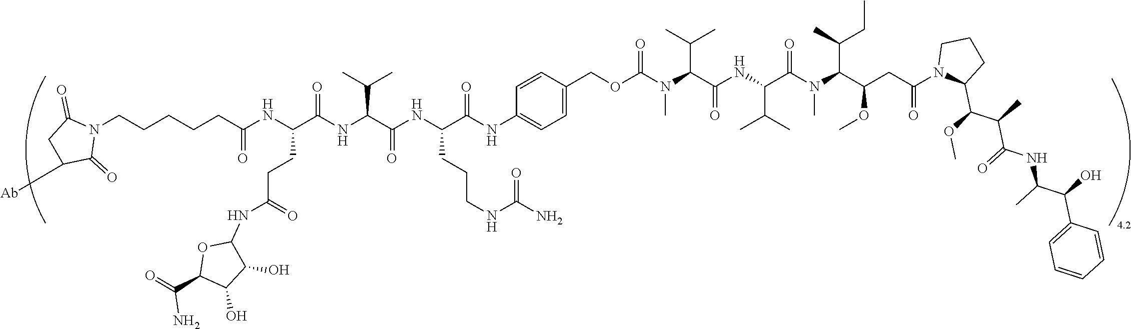 |
| ADC2.4 | 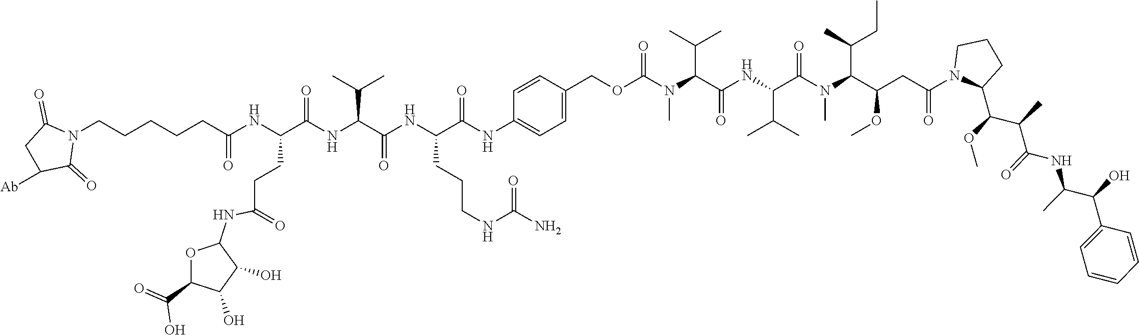 |
| ADC2.6.2 | 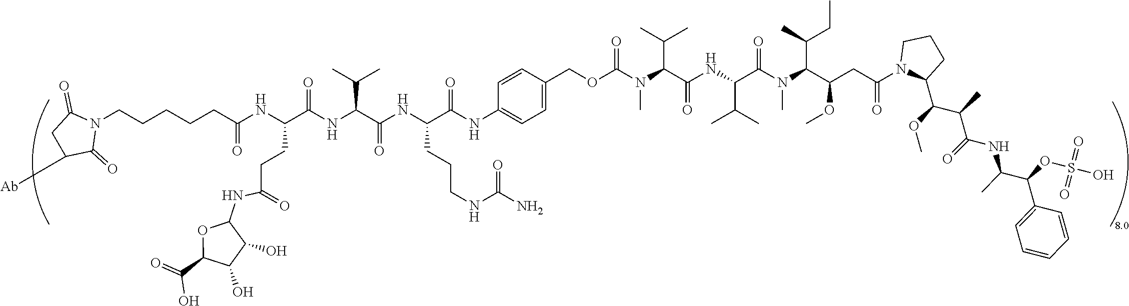 |
| ADC2.7.2 | 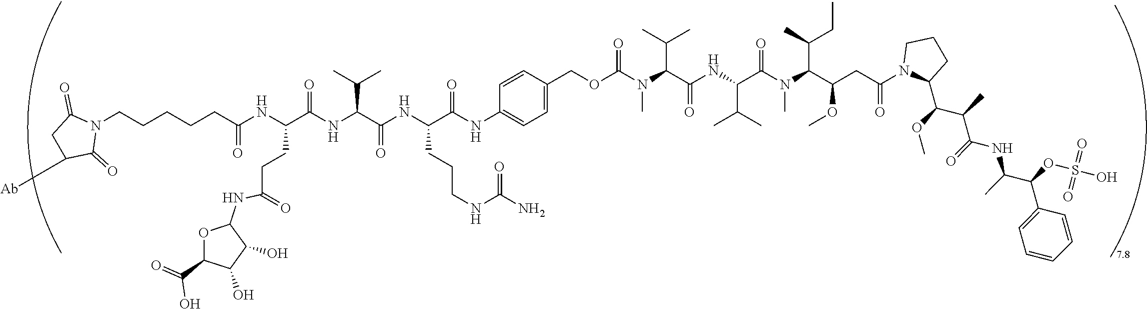 |
| ADC2.9.1 | 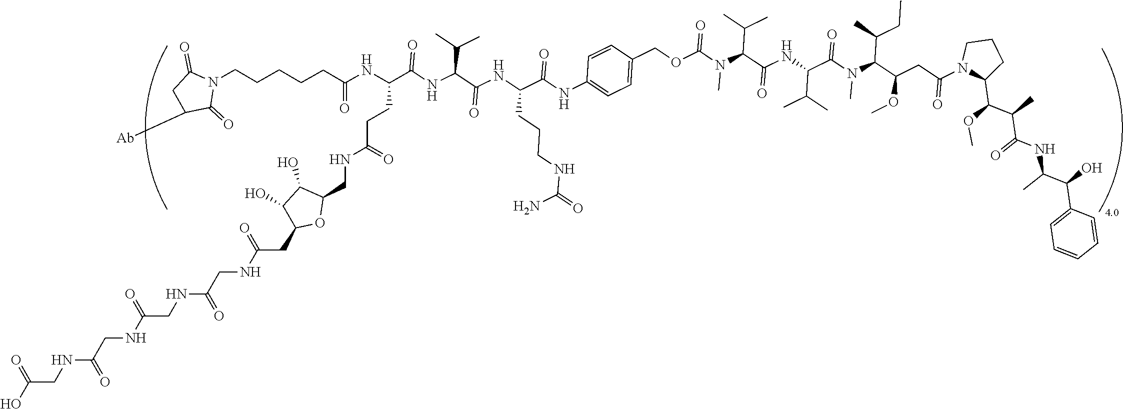 |
| ADC2.9.2 | 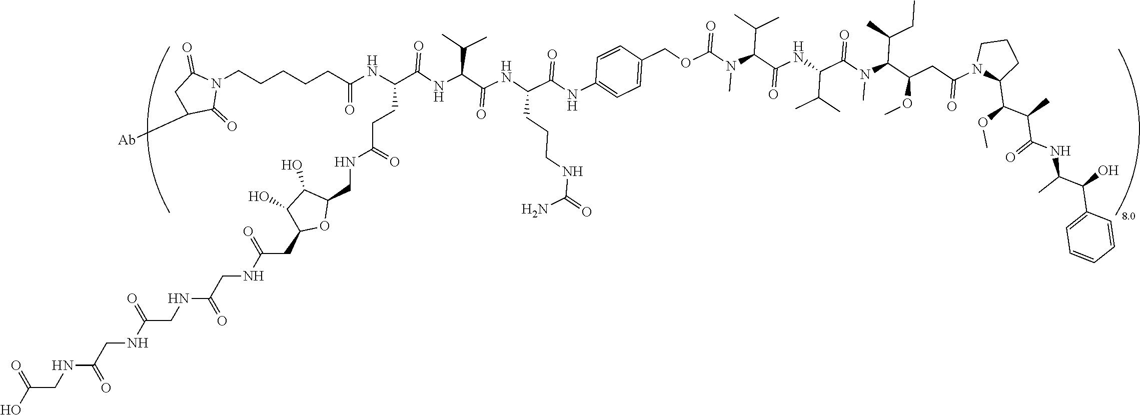 |
| ADC2.10 | 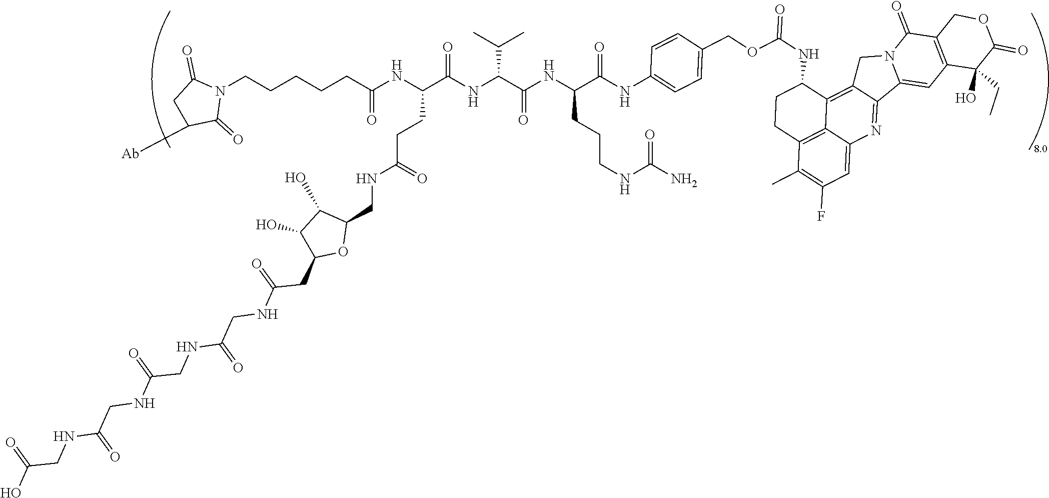 |
| ADC2.11 | 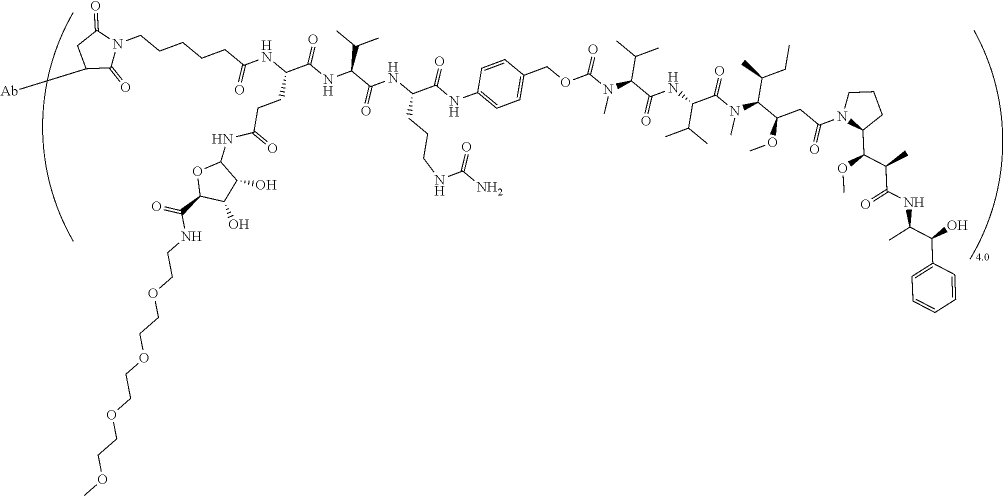 |
| ADC2.22 | 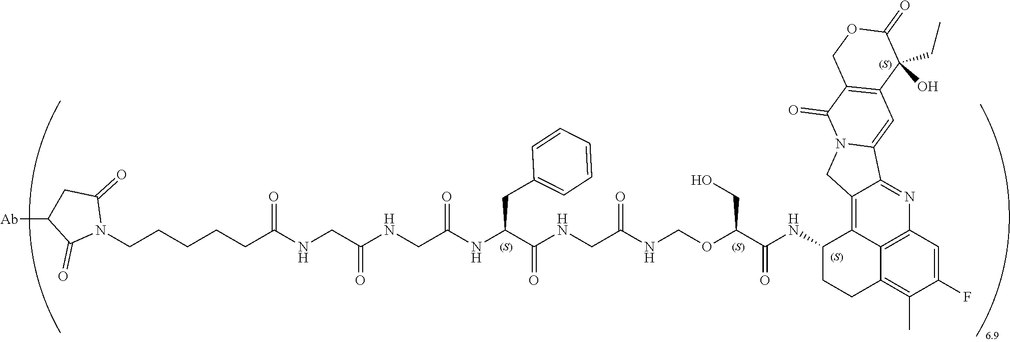 |
| ADC2.23 | 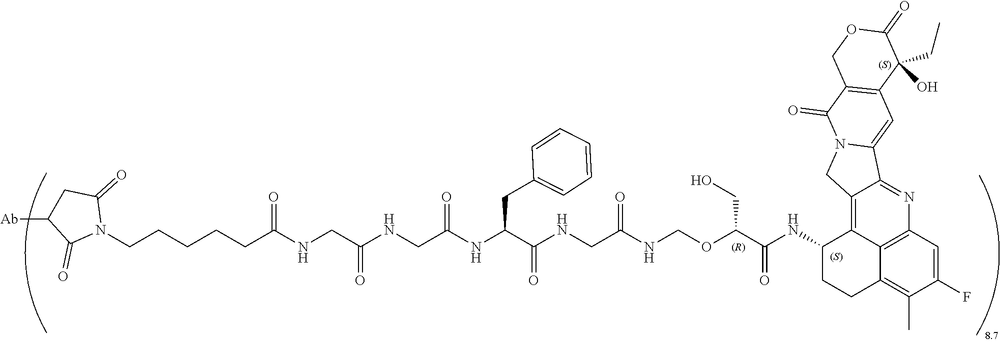 |
| ADC2.24 | 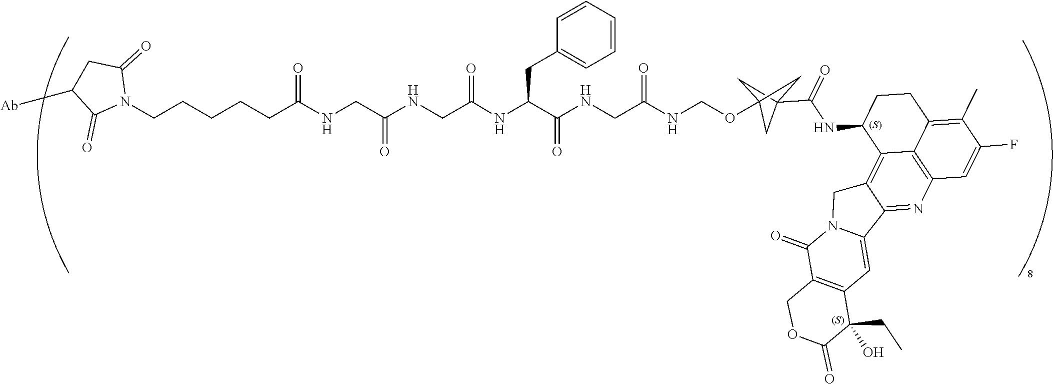 |
| ADC2.25 | 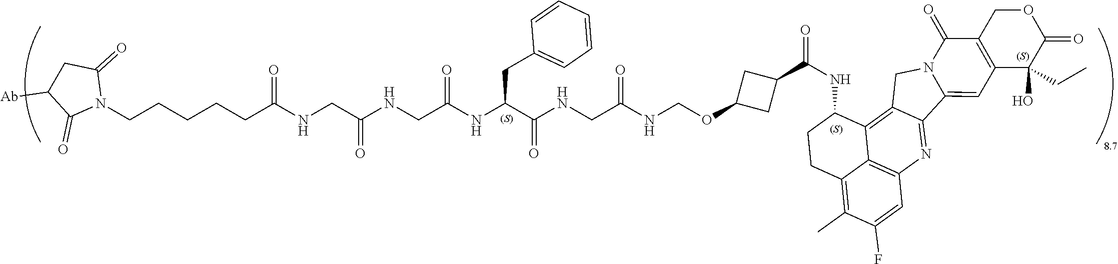 |
| ADC2.26 | 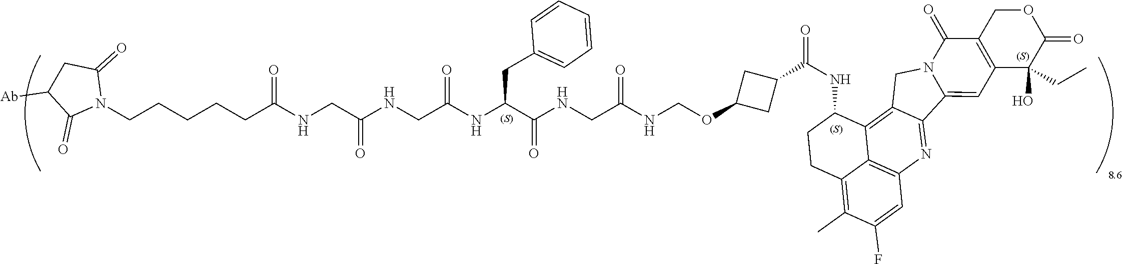 |
| ADC2.27 | 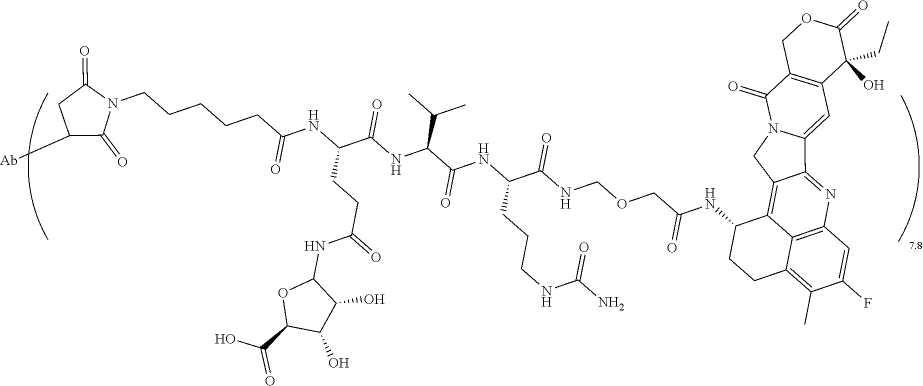 |
| ADC2.29 | 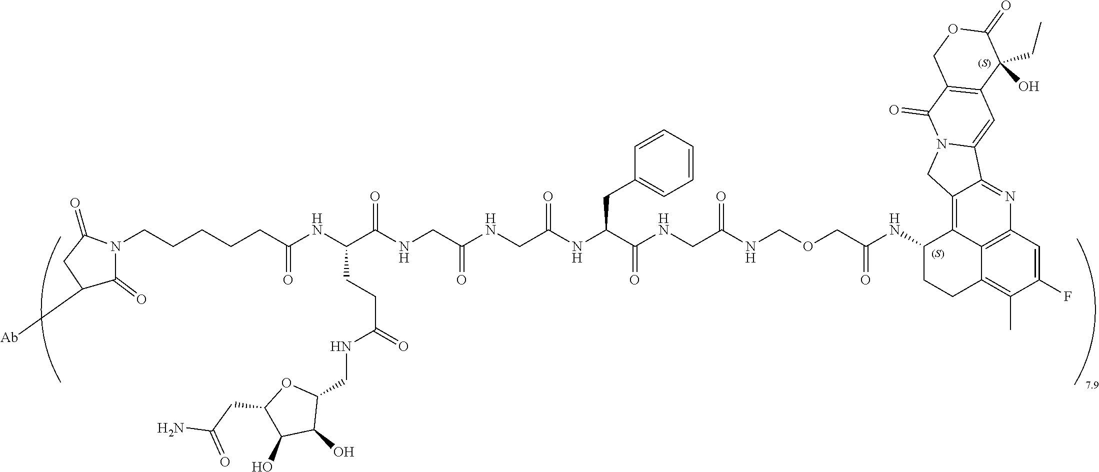 |
| ADC2.30 | 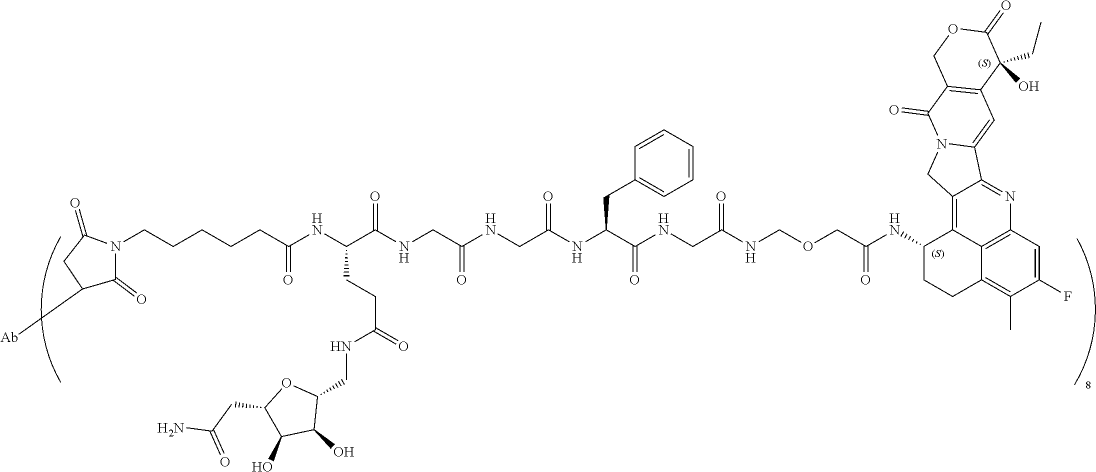 |
| ADC2.31 | 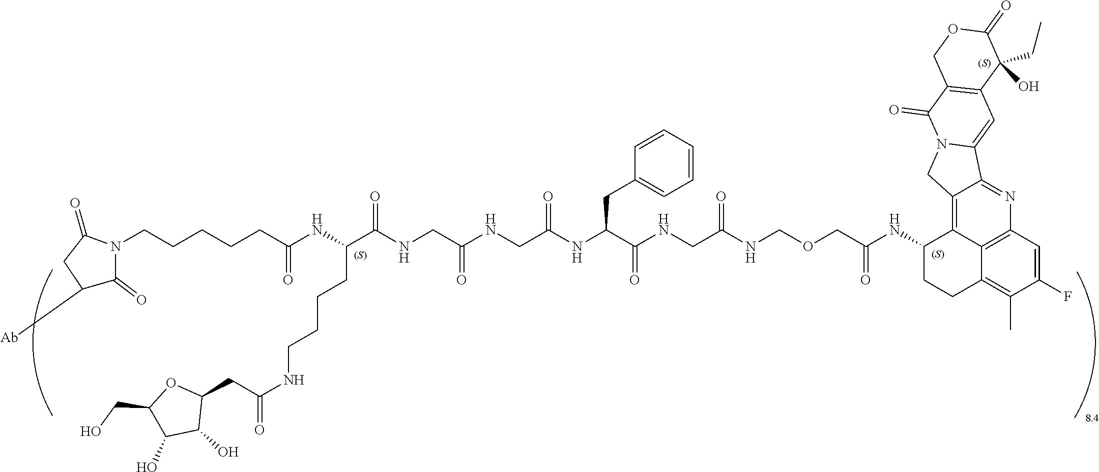 |
| ADC2.32 | 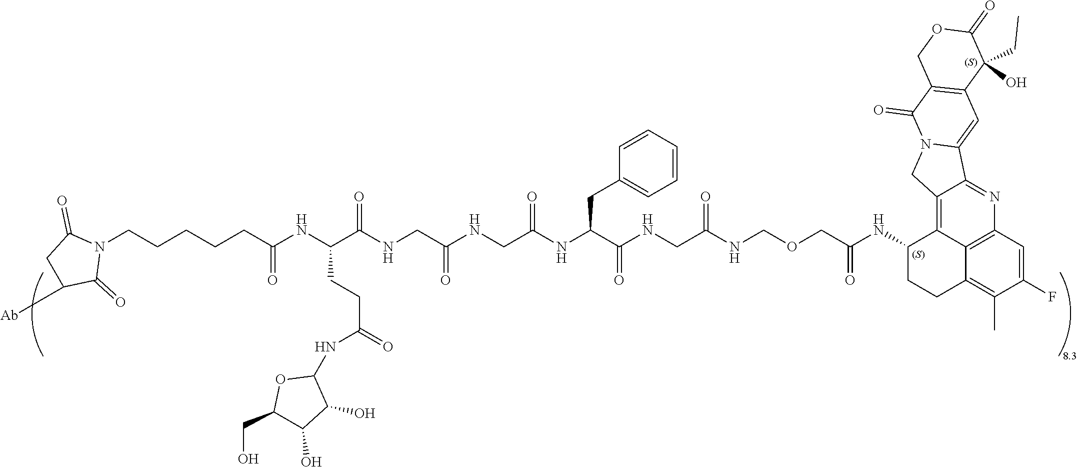 |
| ADC2.33 | 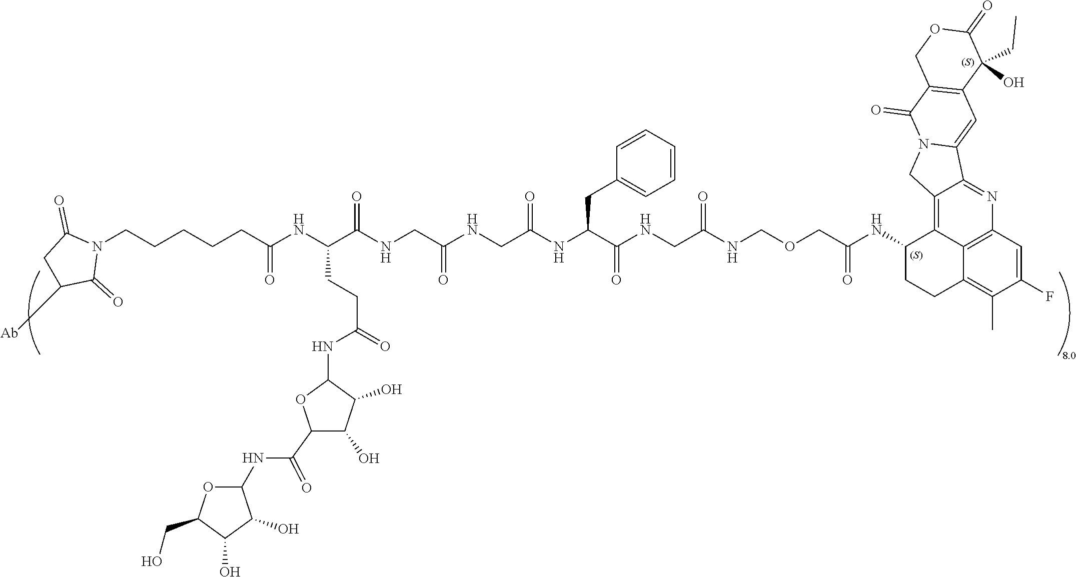 |
| ADC2.34 | 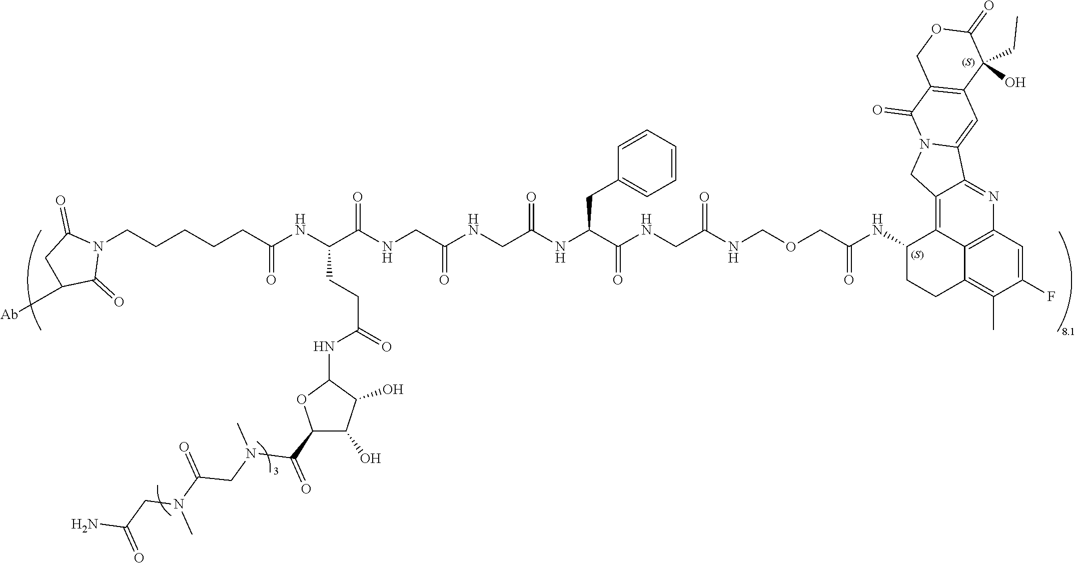 |
| ADC2.35 | 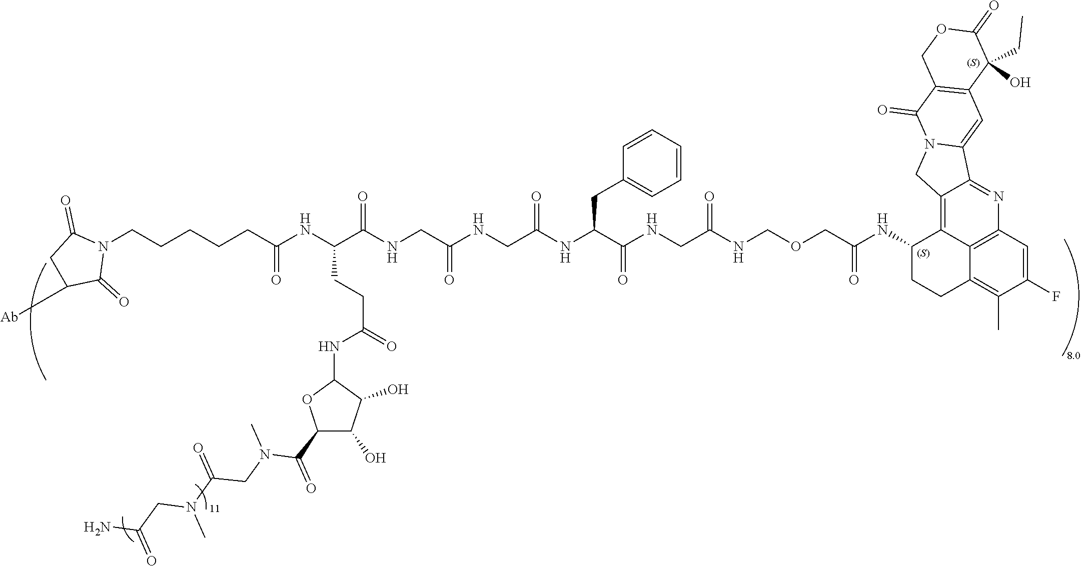 |
| ADC2.36.1 | 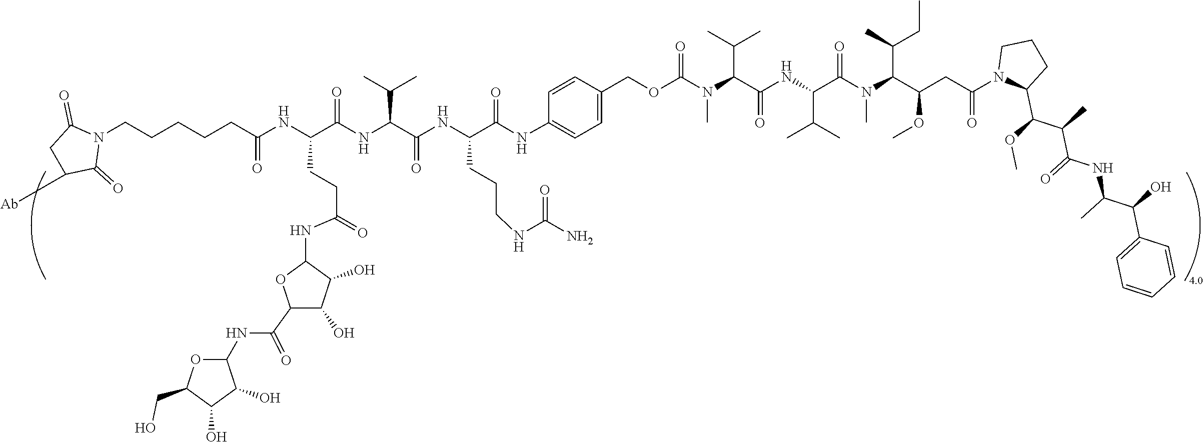 |
| ADC2.36.2 | 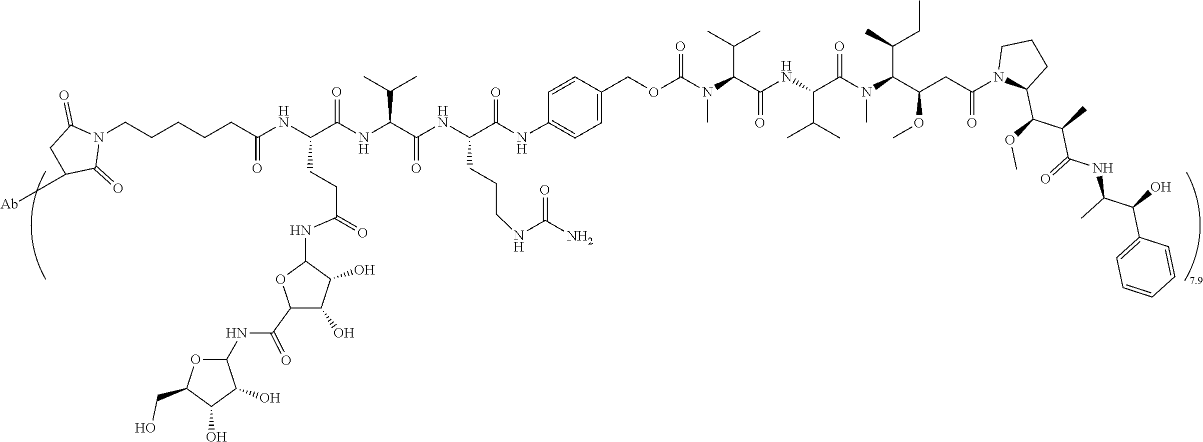 |
| ADC2.37 | 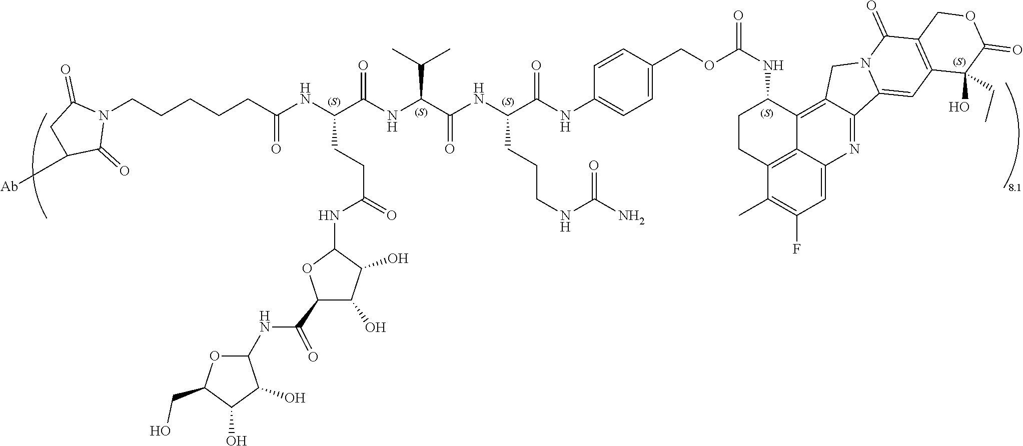 |
| ADC2.38 | 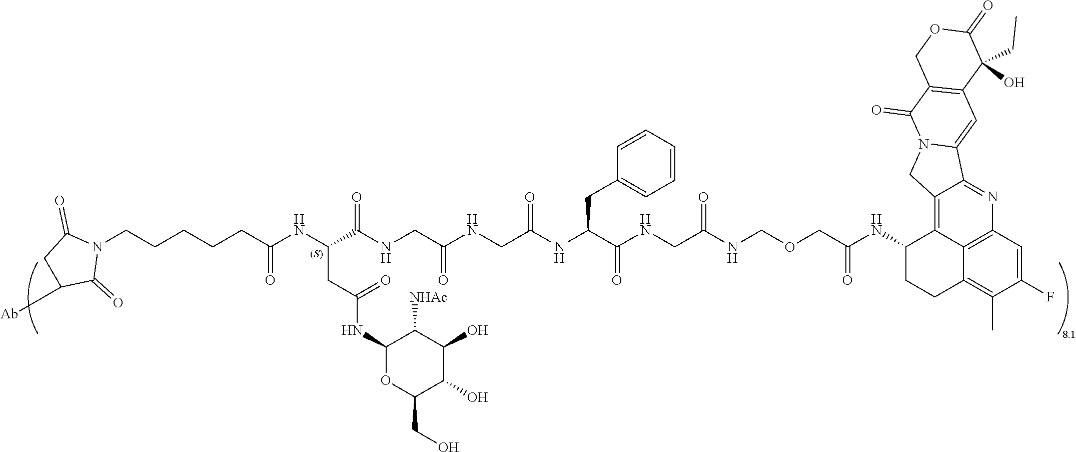 |
| ADC2.39 | 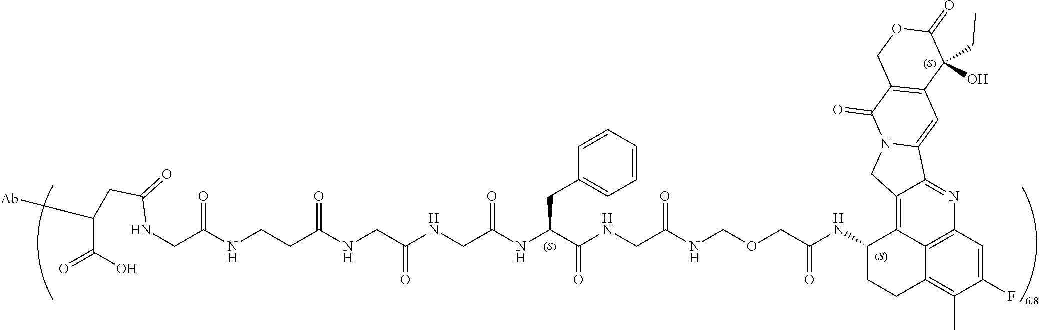 |
| ADC2.40 | 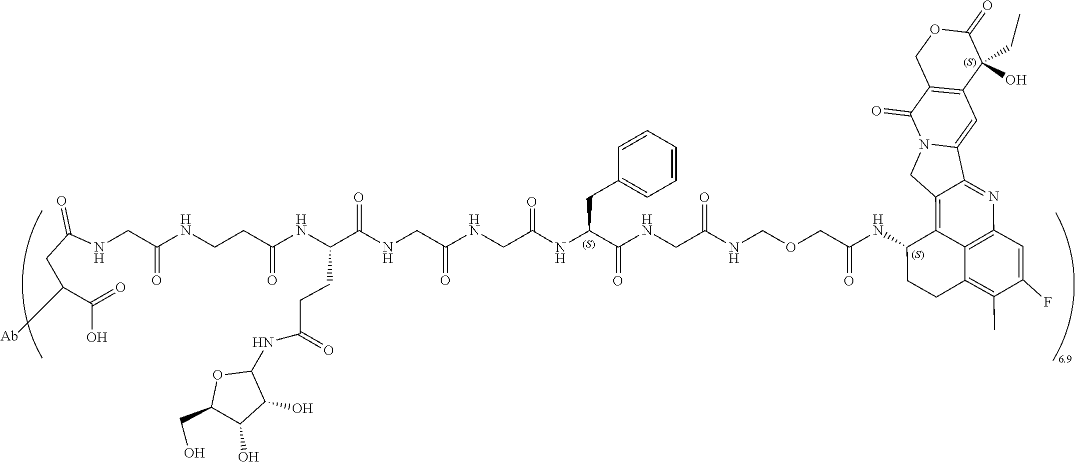 |
| ADC2.41 | 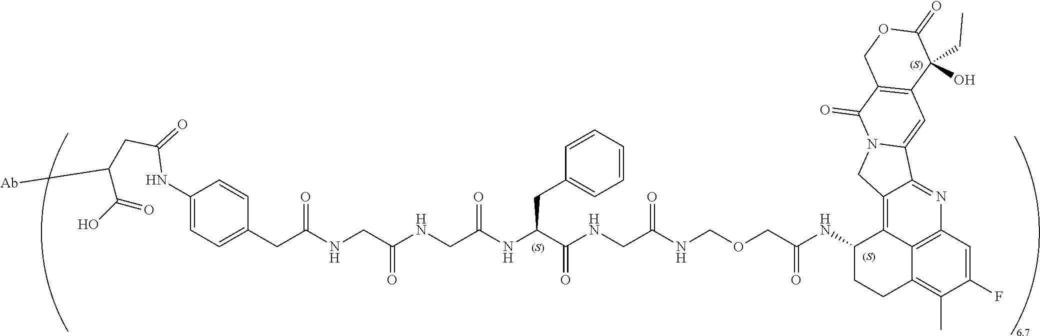 |
| ADC2.42 | 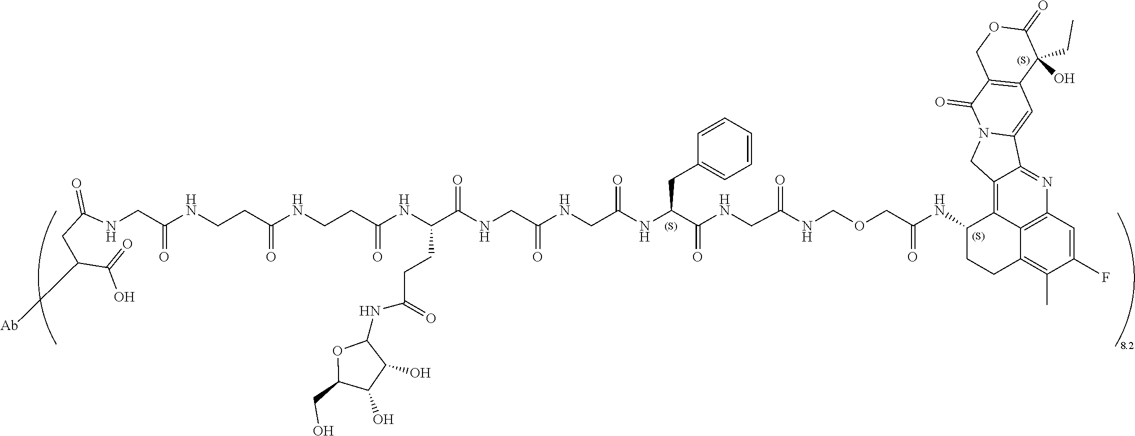 |
| ADC2.42.RO | 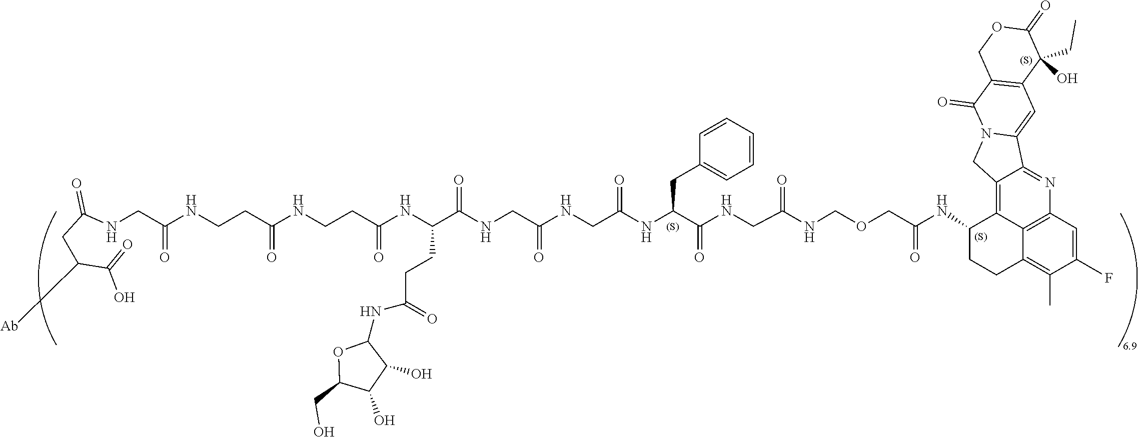 |
| ADC2.43 | 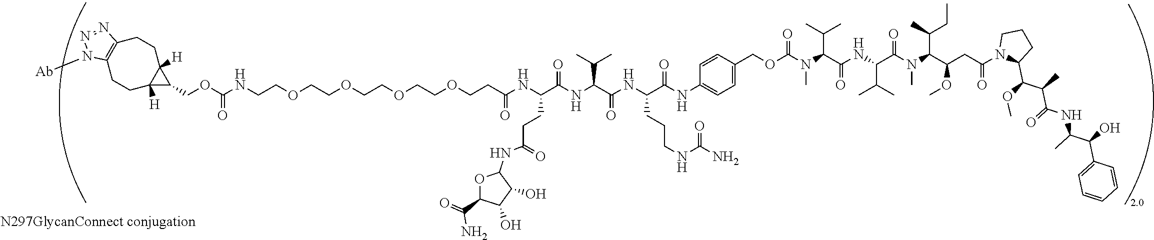 |
| ADC2.44 | 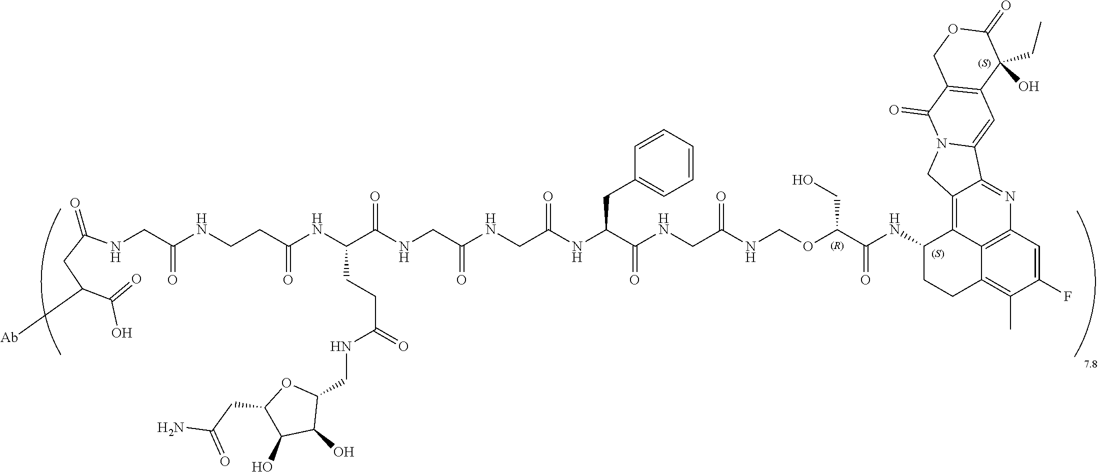 |
| ADC2.45 | 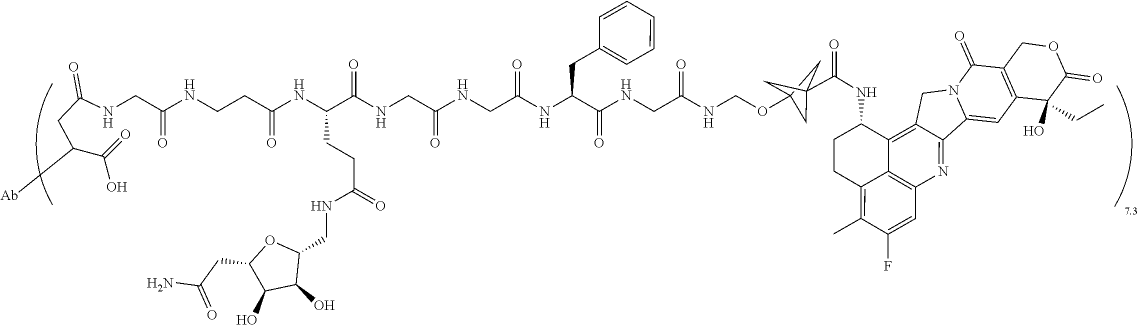 |
| ADC2.46 | 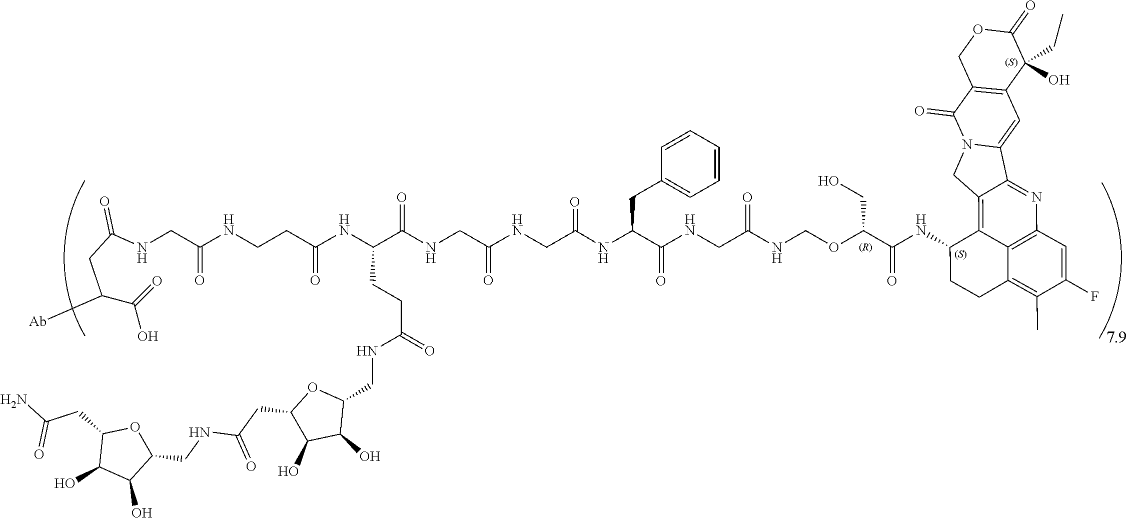 |
| ADC2.47 | 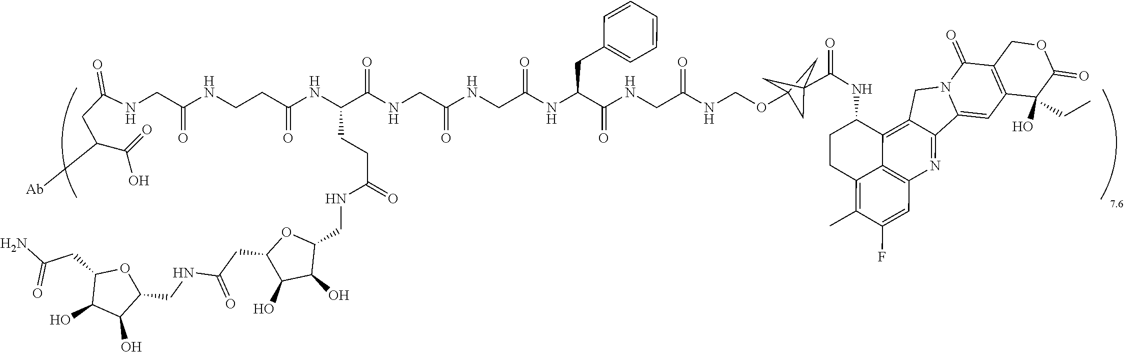 |
| ADC2.48 | 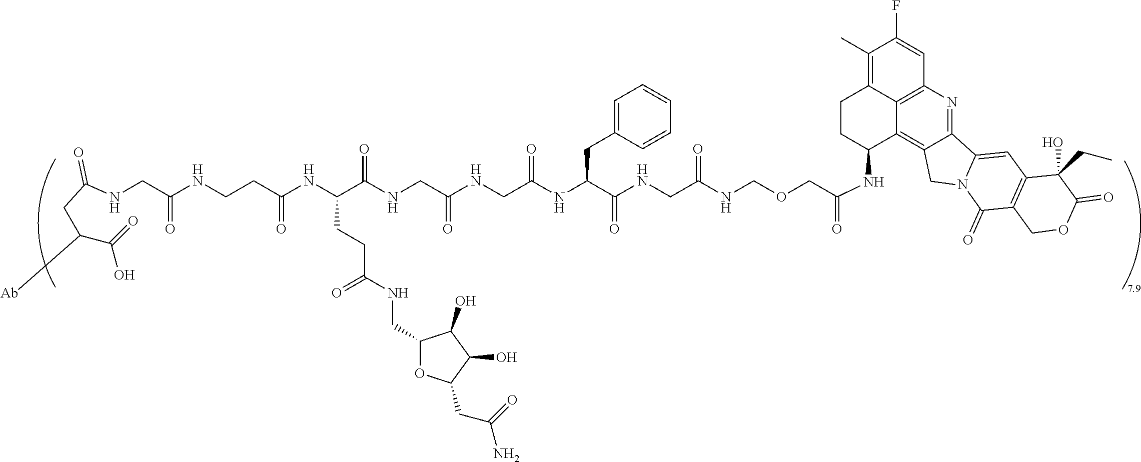 |
| ADC2.49 | 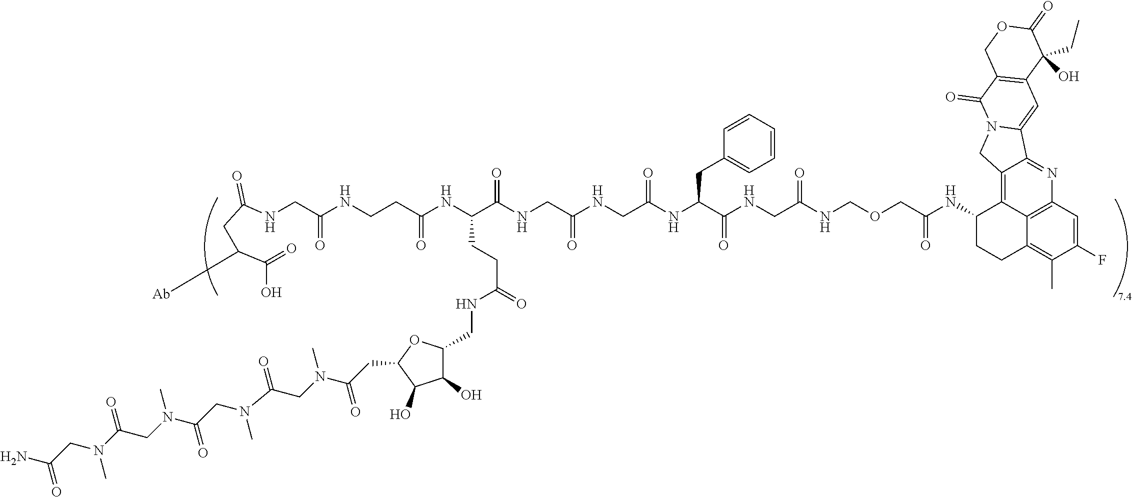 |
| ADC2.50 | 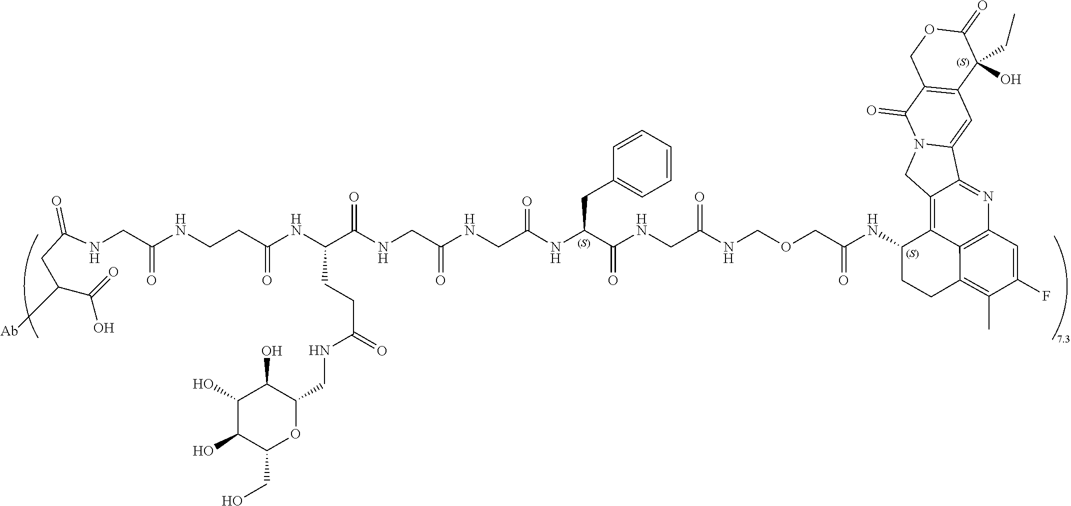 |
| ADC2.51 | 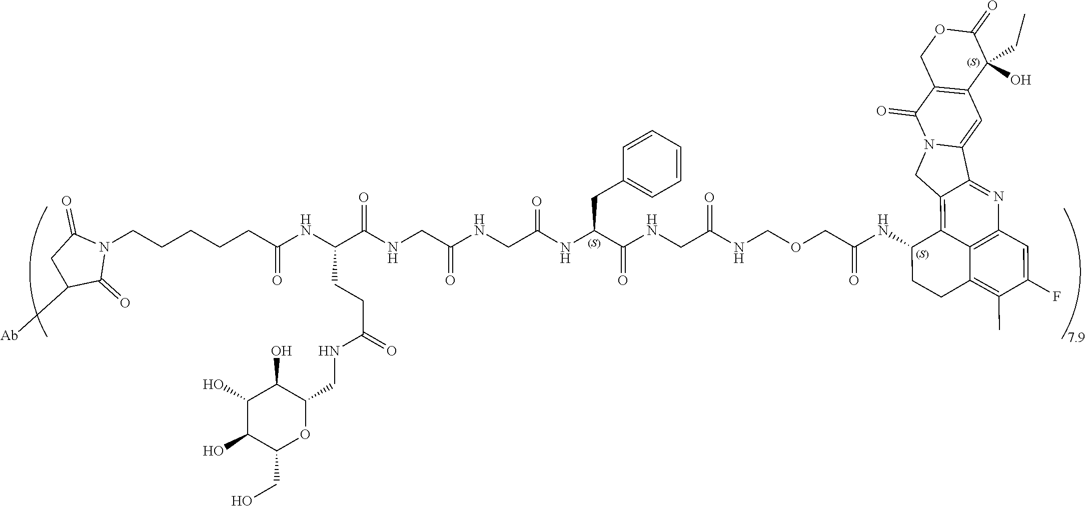 |
| ADC2.53 | 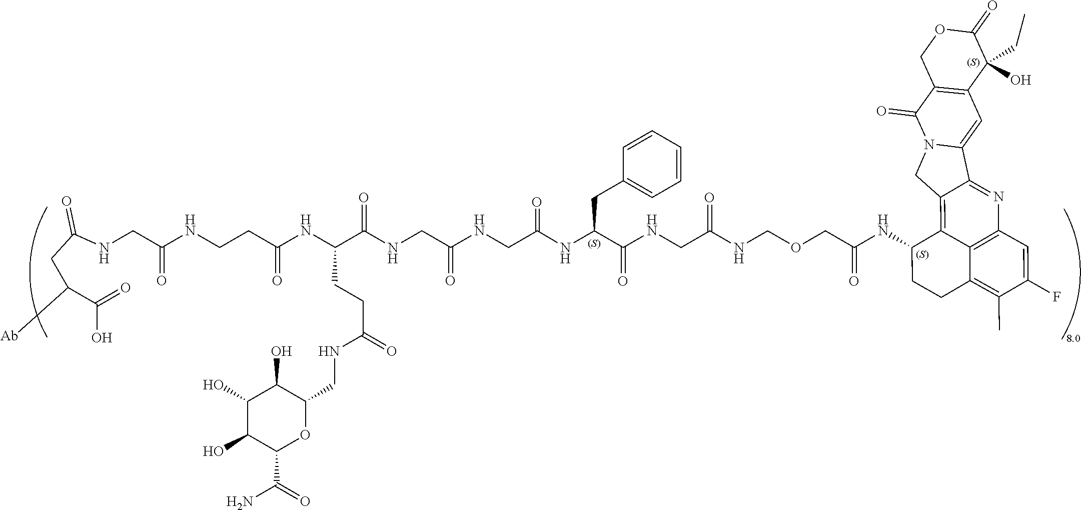 |
| ADC2.54 | 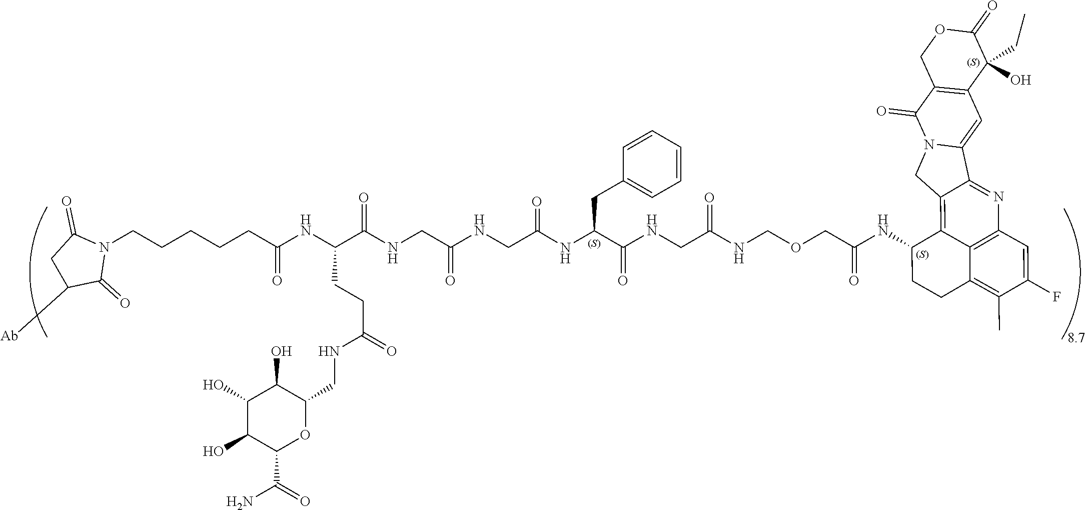 |
| ADC2.55 | 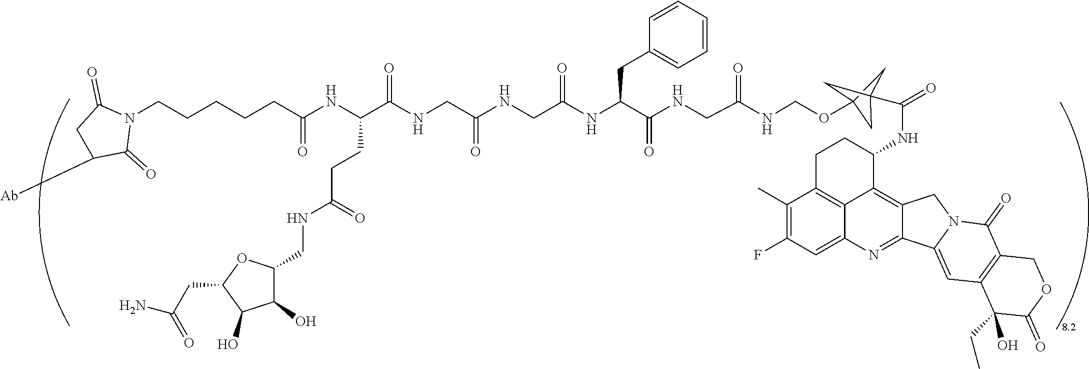 |
| ADC2.56 | 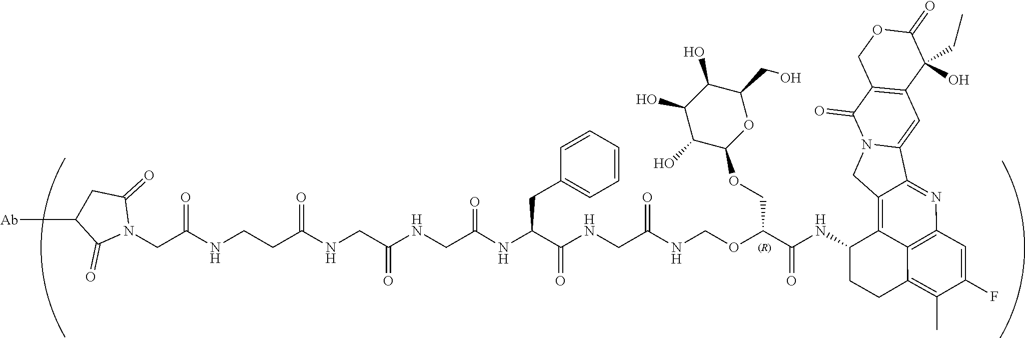 |
| ADC2.57 | 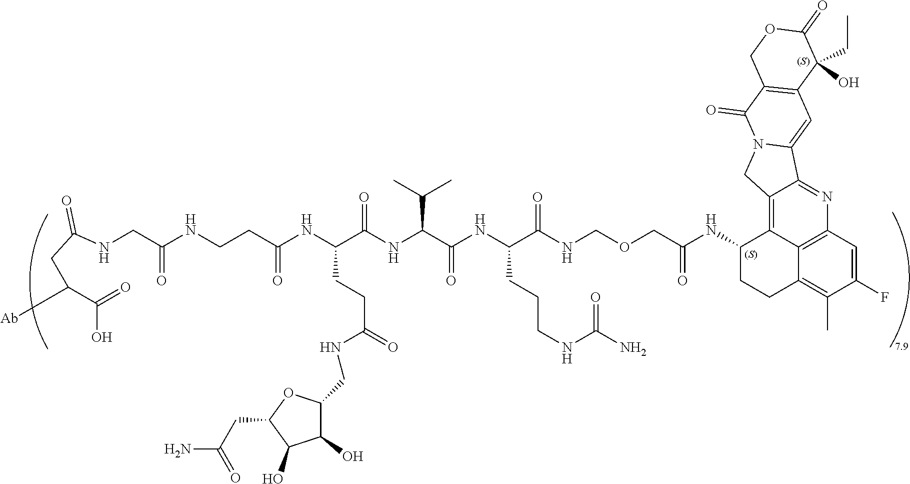 |
| ADC2.58 | 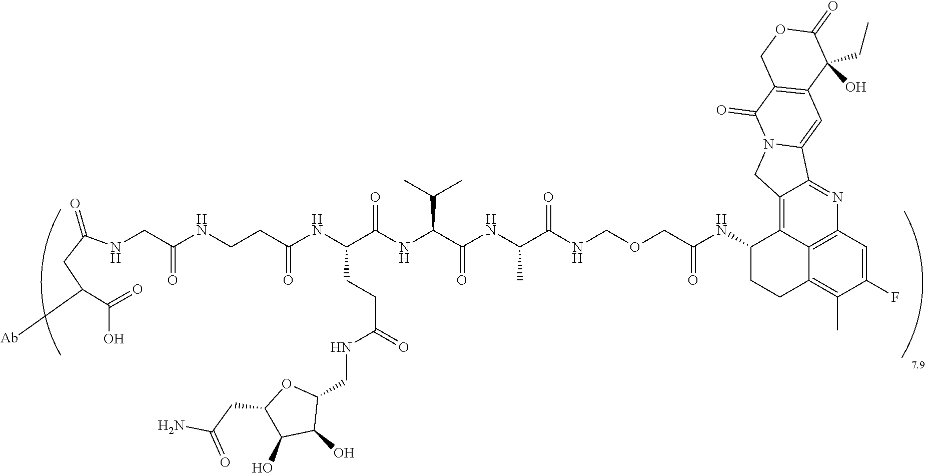 |
| ADC2.59 | 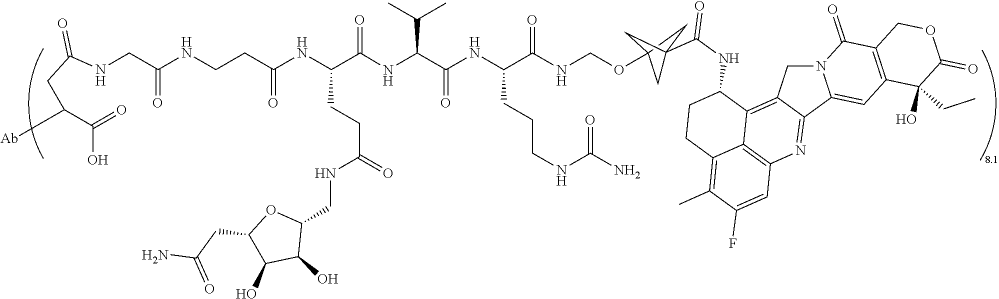 |
| ADC2.60 | 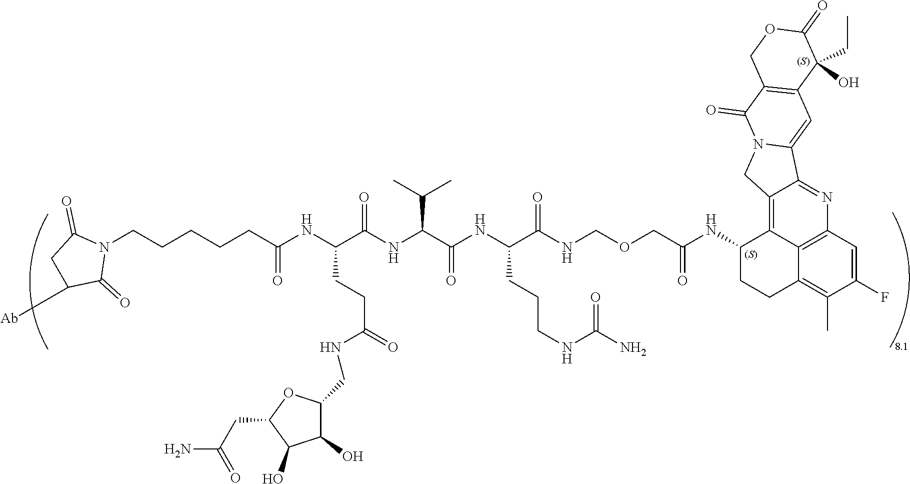 |
| ADC2.61 | 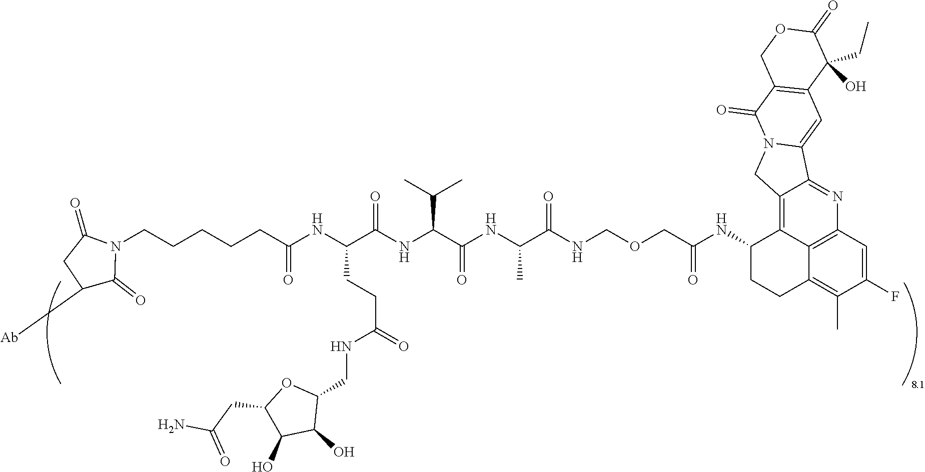 |
| ADC2.62.1 | 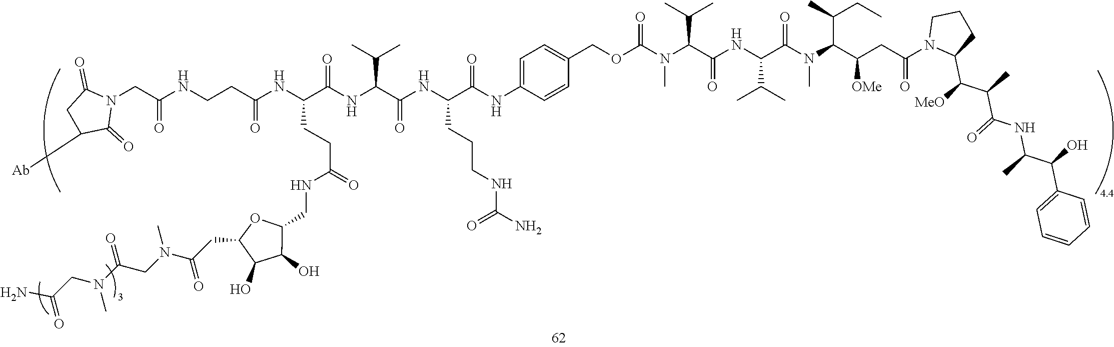 |
| ADC2.62.2 | 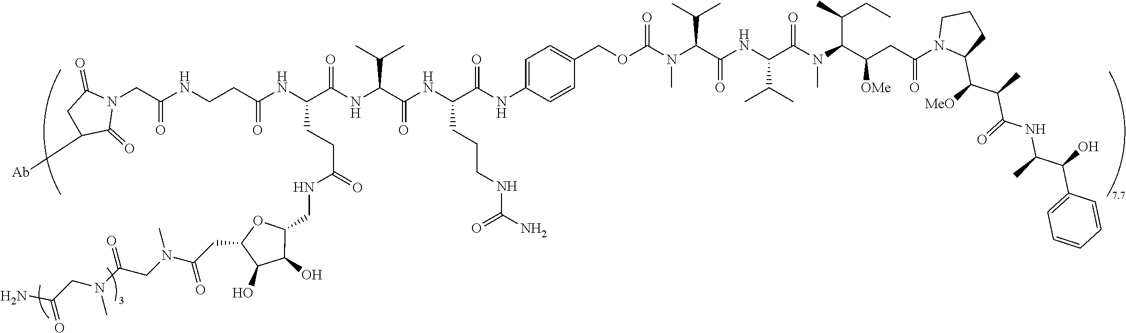 |
| ADC2.63.1 | 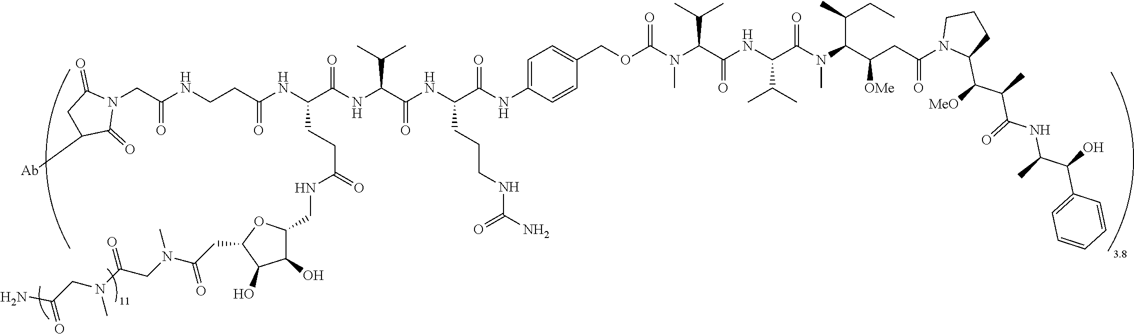 |
| ADC2.63.2 | 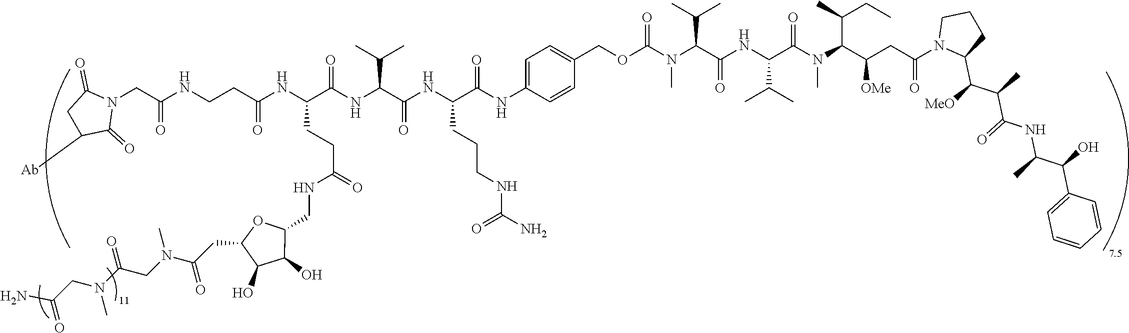 |
| ADC2.64.1 | 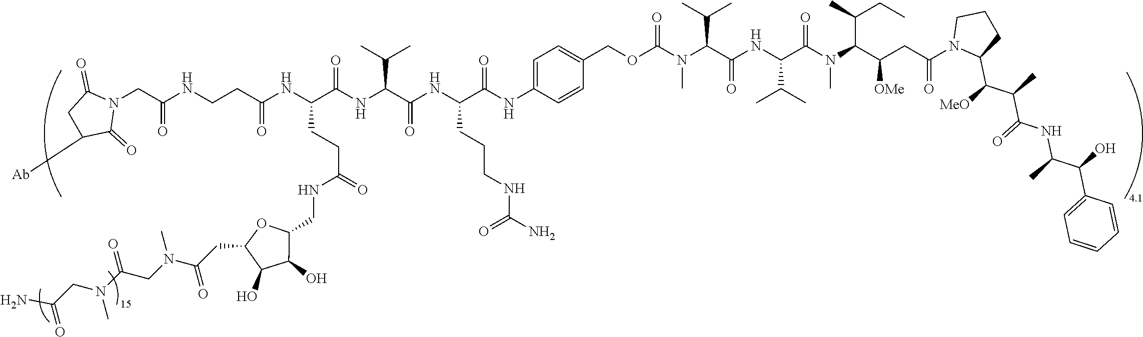 |
| ADC2.64.2 | 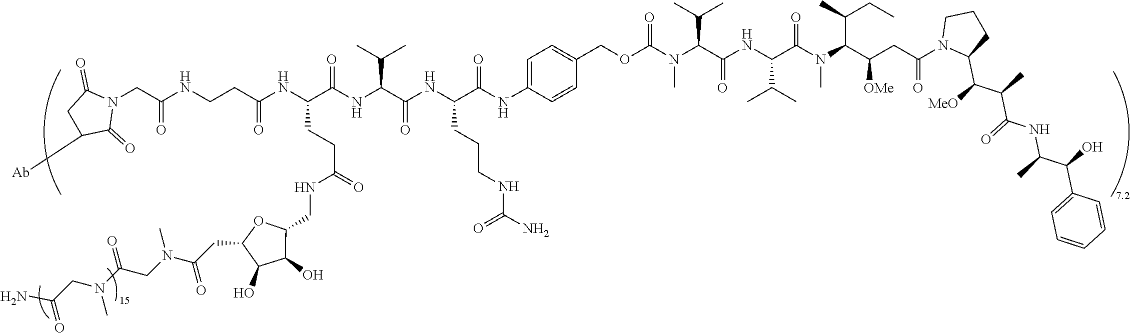 |
| ADC2.65.1 | 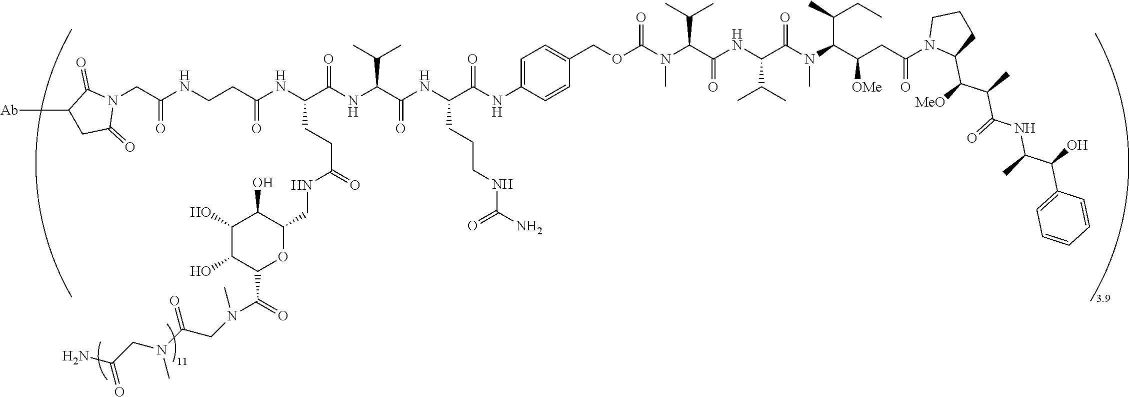 |
| ADC2.65.2 | 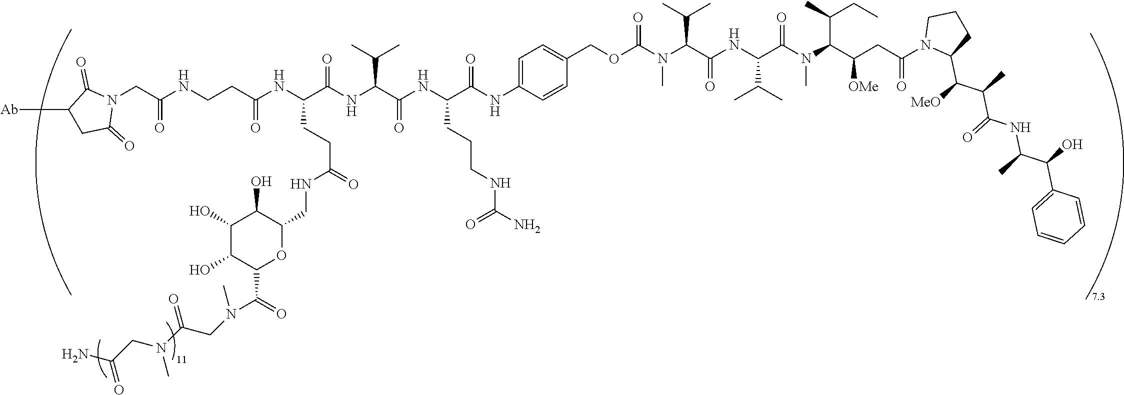 |
| ADC3.1 | 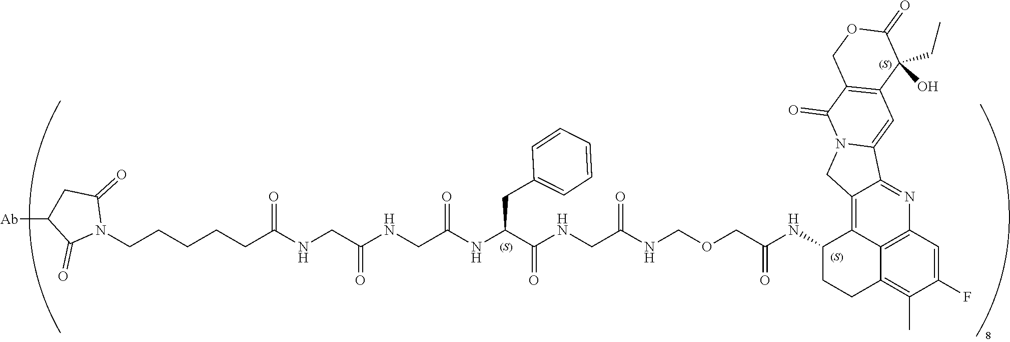 |
| ADC3.2 | 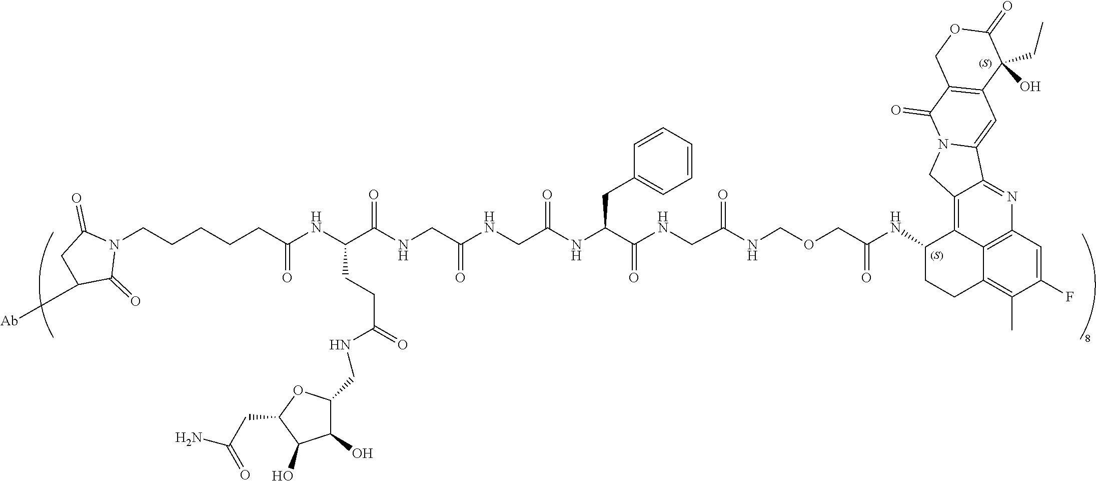 |
| ADC3.3 | 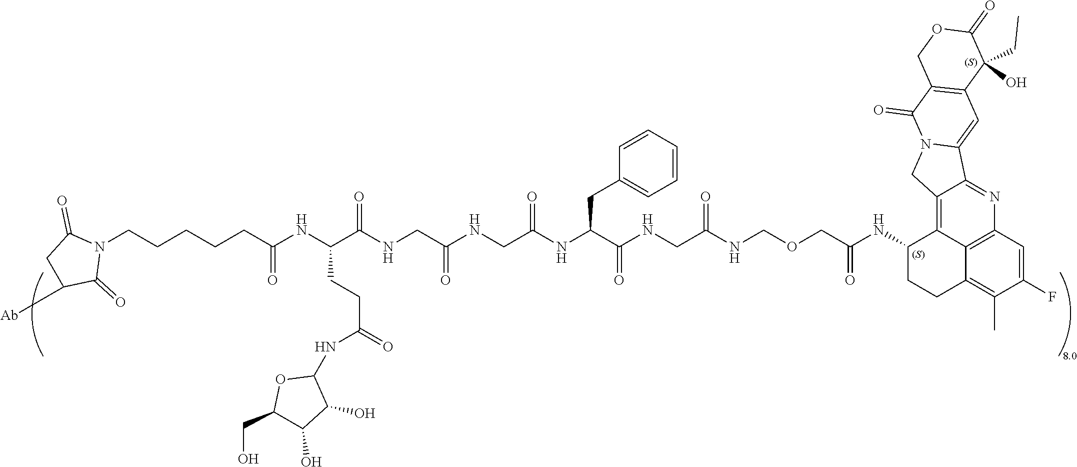 |
| ADC3.4 | 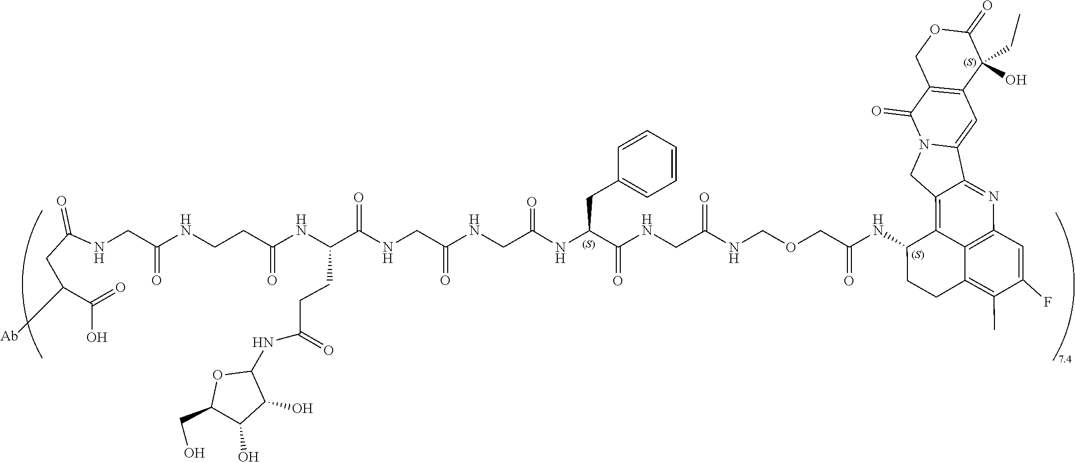 |
| ADC3.5 | 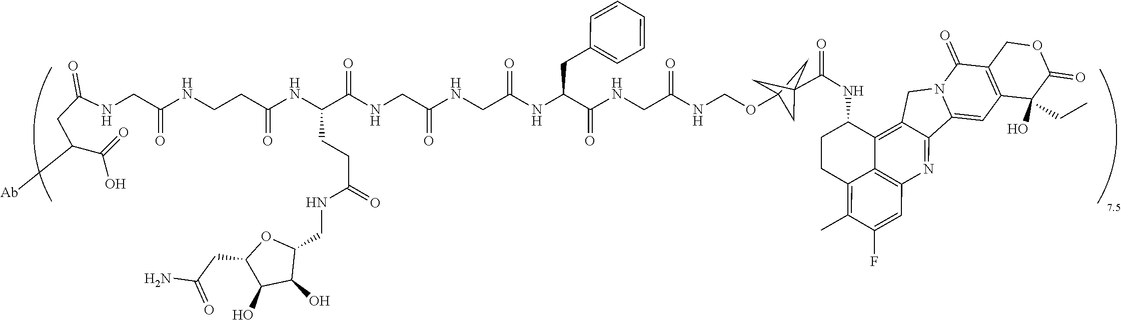 |
| ADC3.6 | 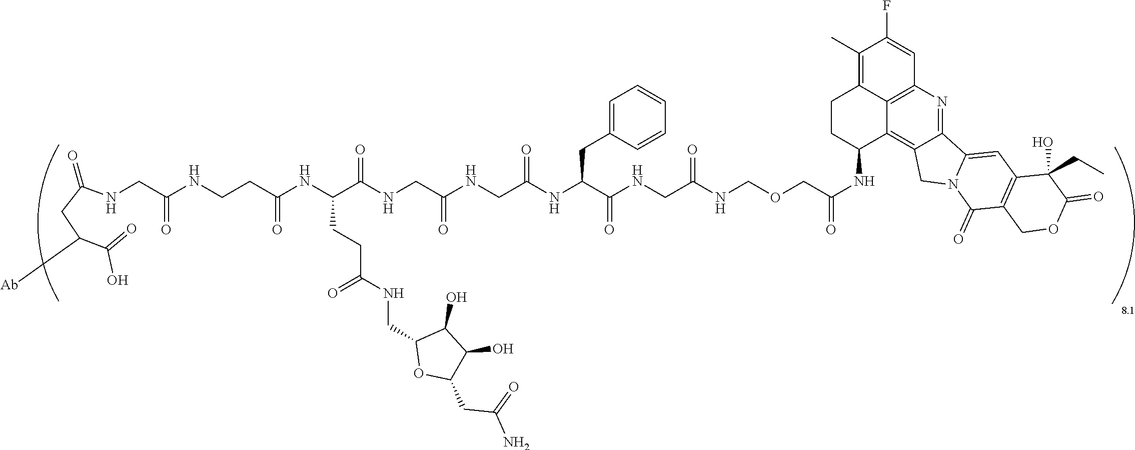 |
| ADC3.7 | 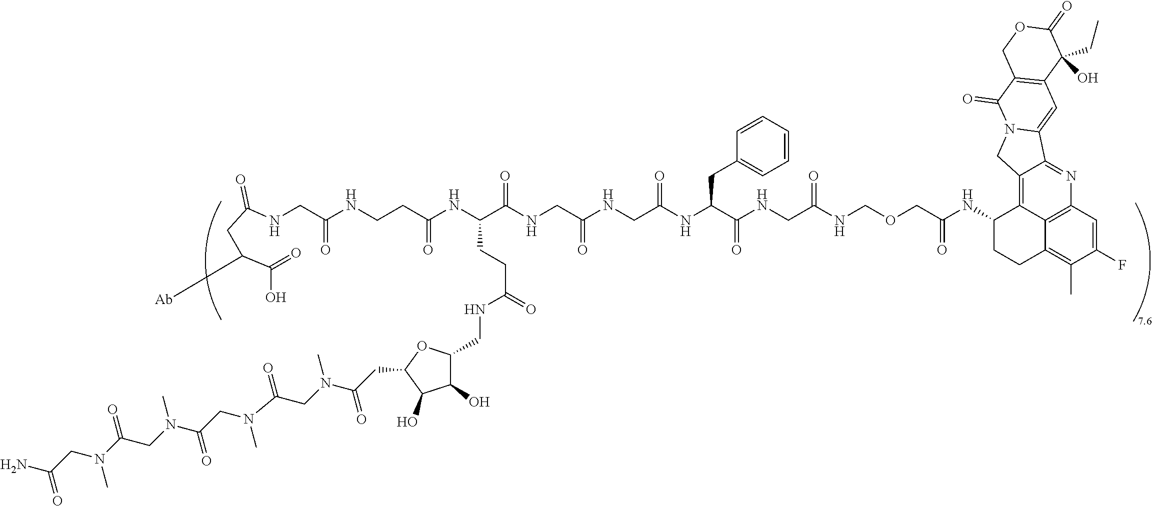 |
| ADC3.8 | 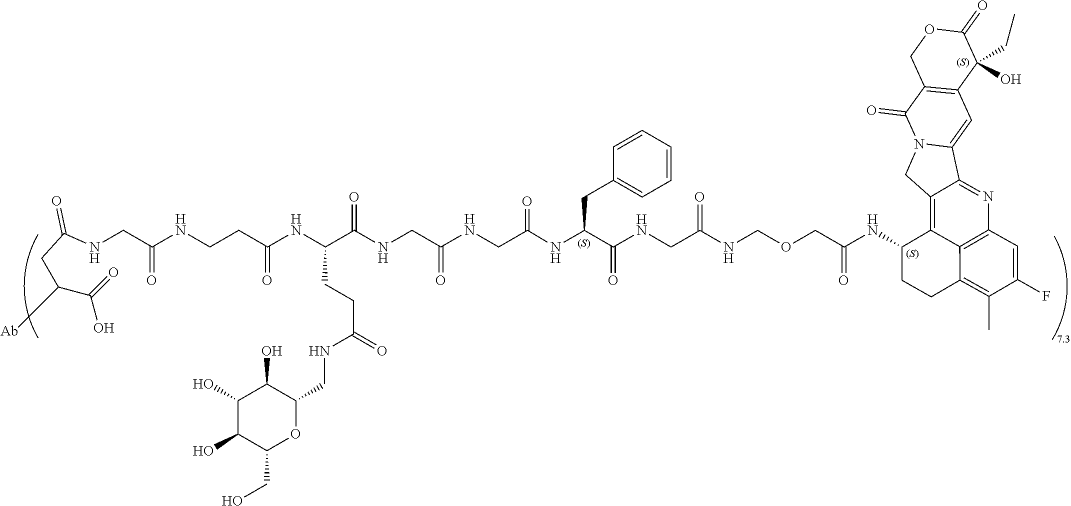 |
| ADC3.9 | 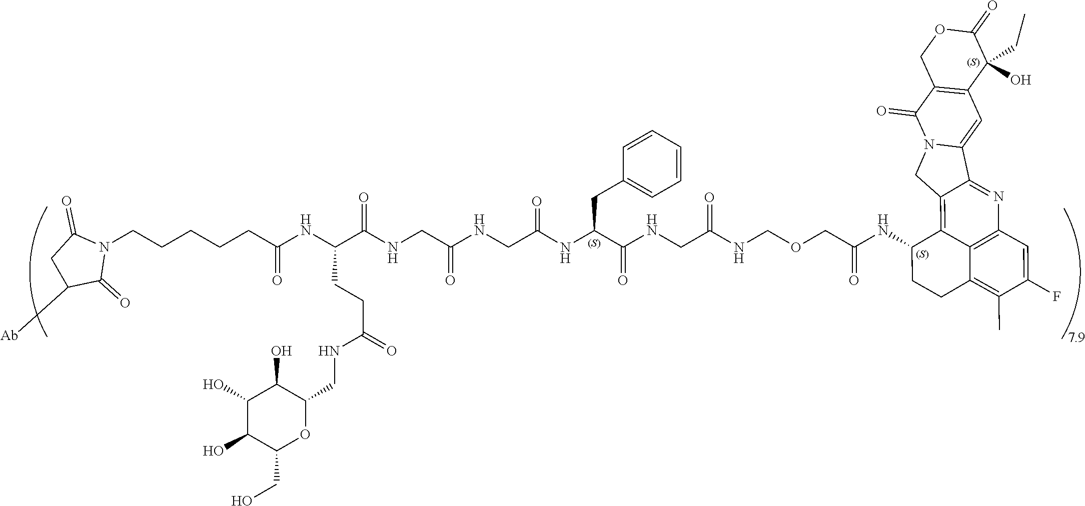 |
| ADC3.10 | 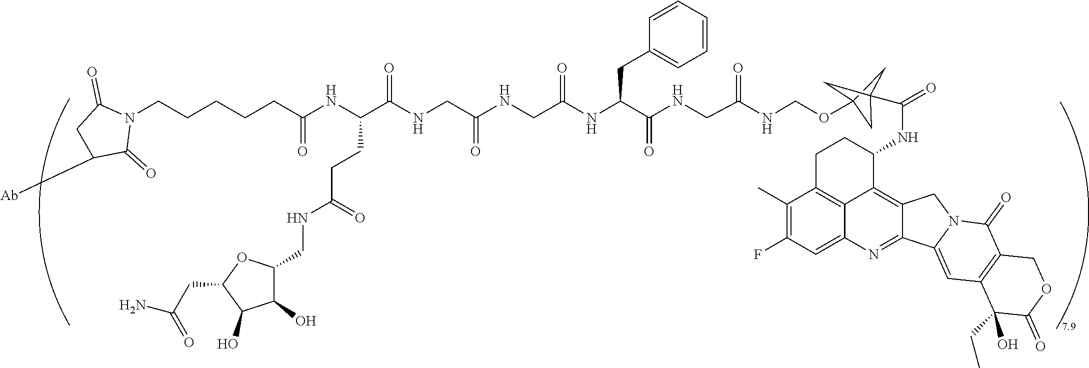 |
| ADC3.11 | 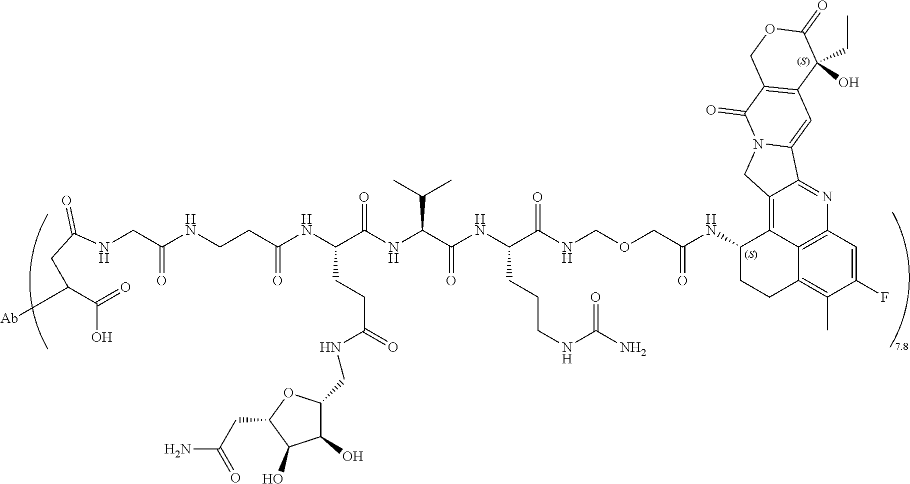 |
| ADC3.12 | 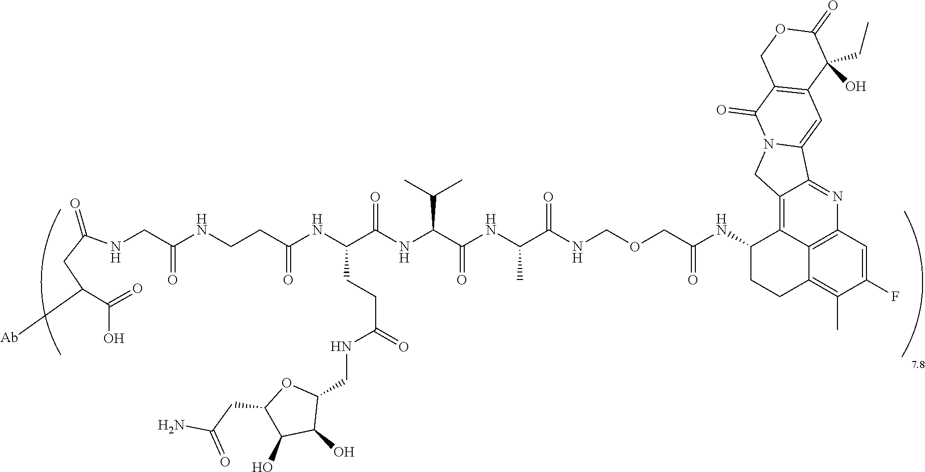 |
| ADC3.13 | 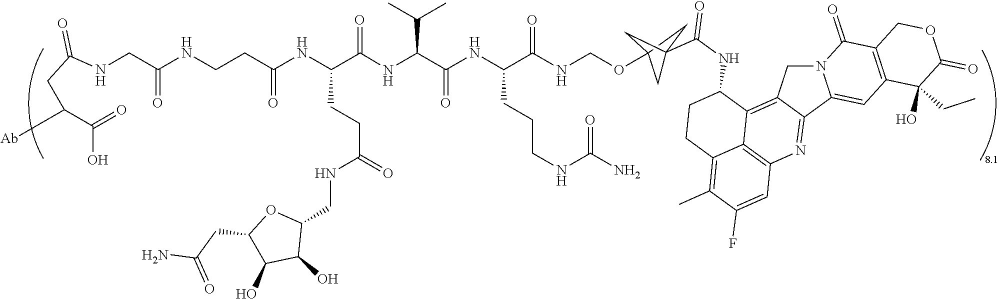 |
| ADC3.14 | 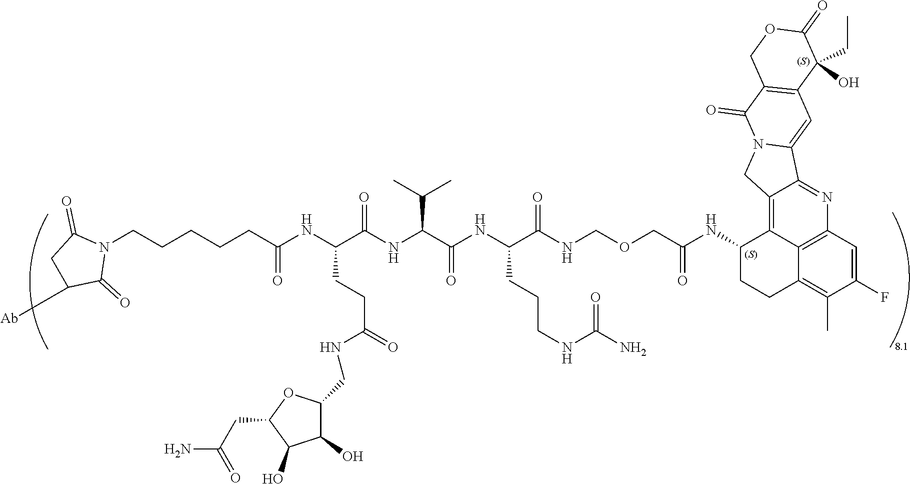 |
| ADC3.15 | 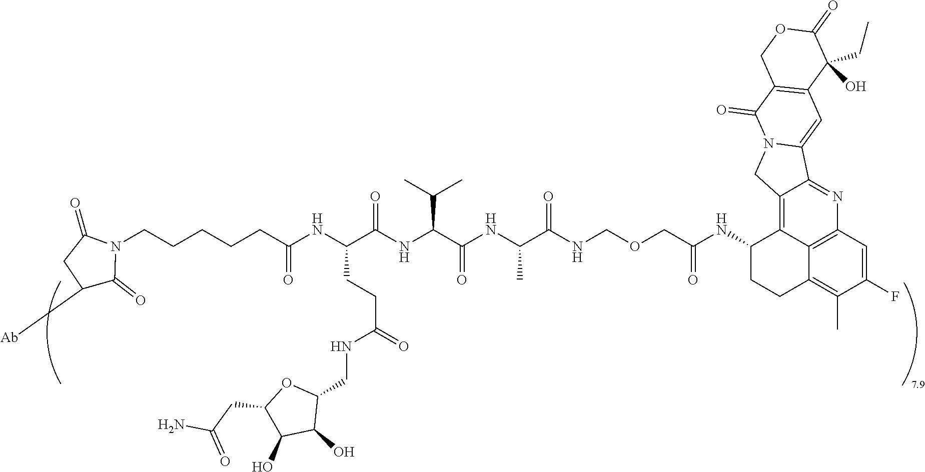 |
| ADC3.16 | 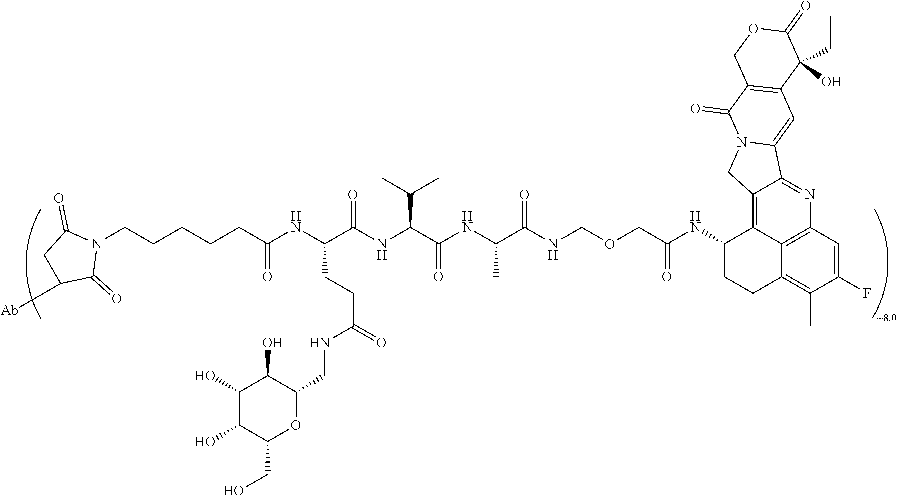 |
| ADC3.17 | 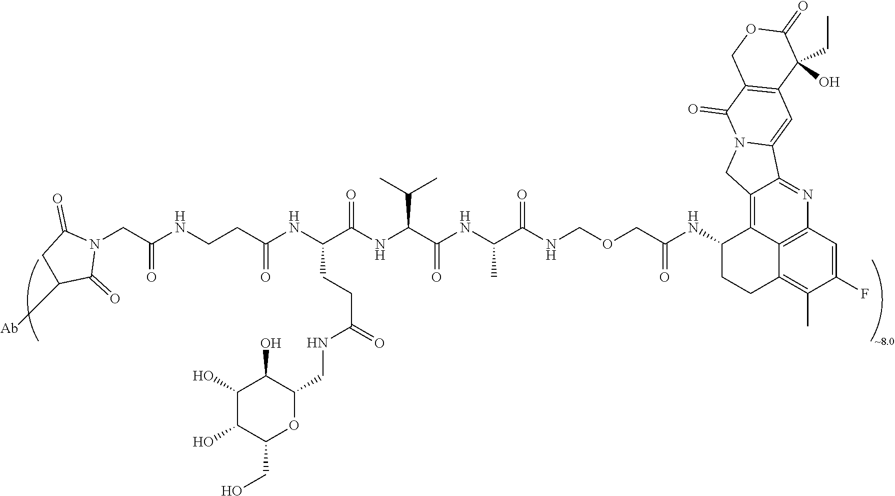 |
| ADC3.18 | 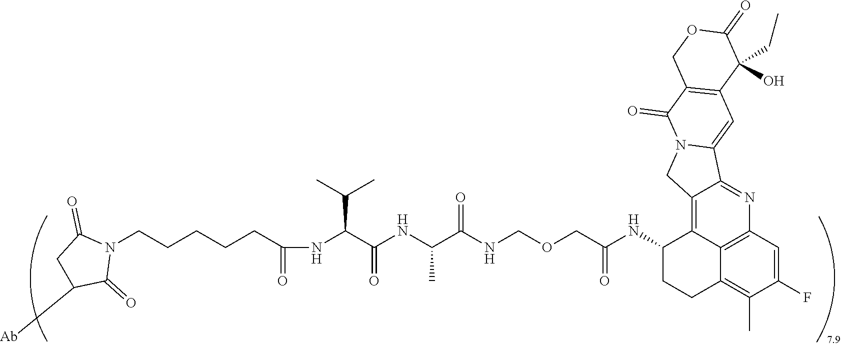 |
| ADC3.19 | 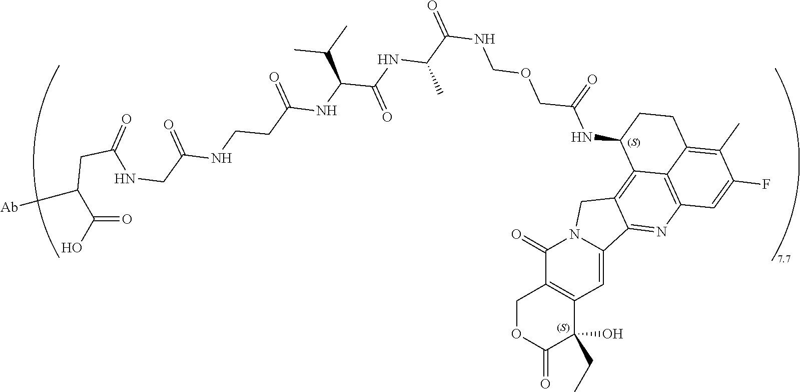 |
| ADC3.20 | 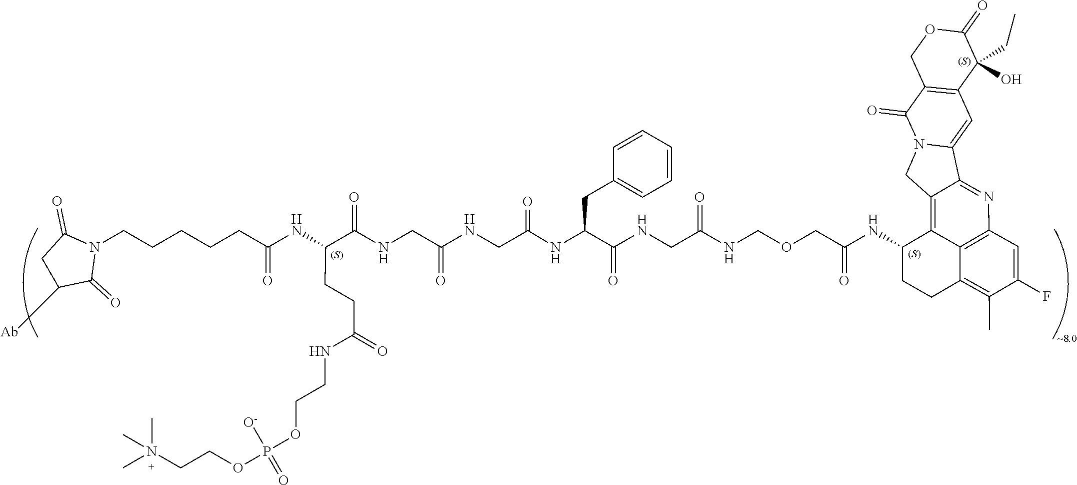 |
| ADCC2 | 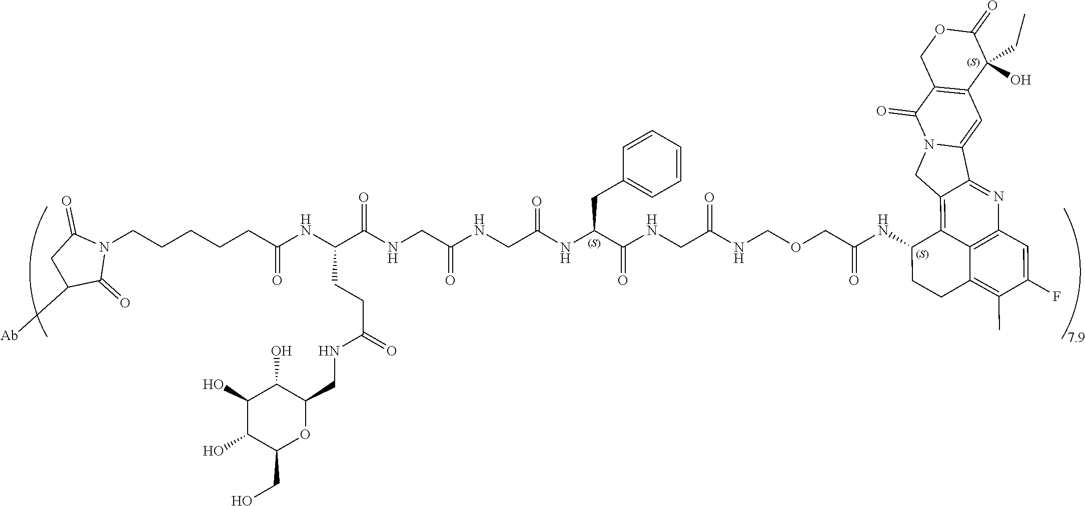 |
| ADCC3 | 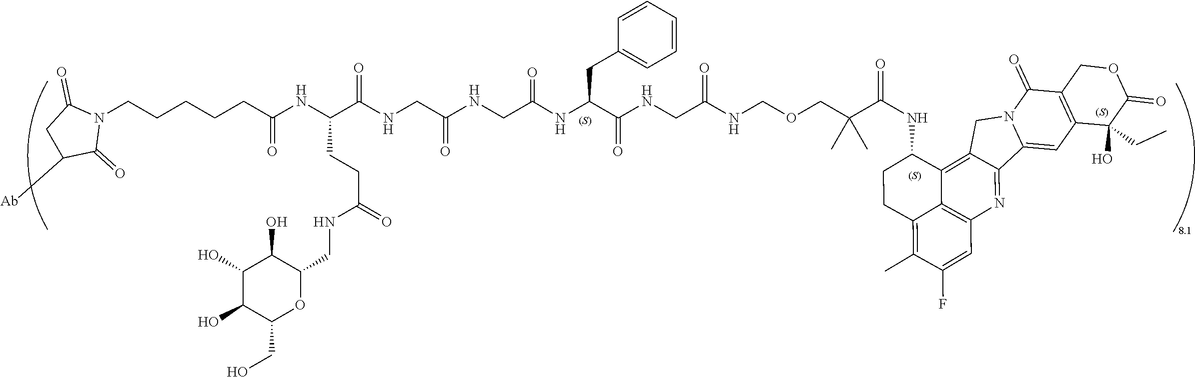 |
| ADCC4 | 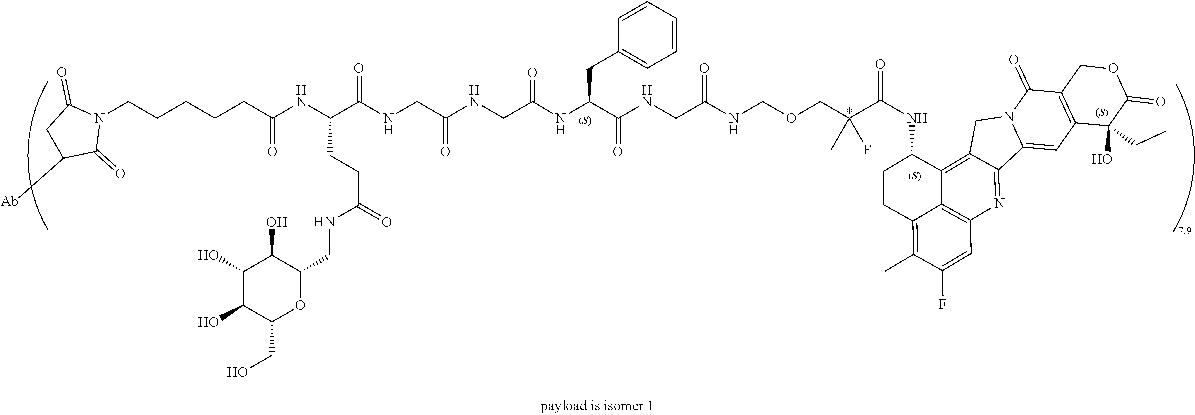 |
| ADCC5 | 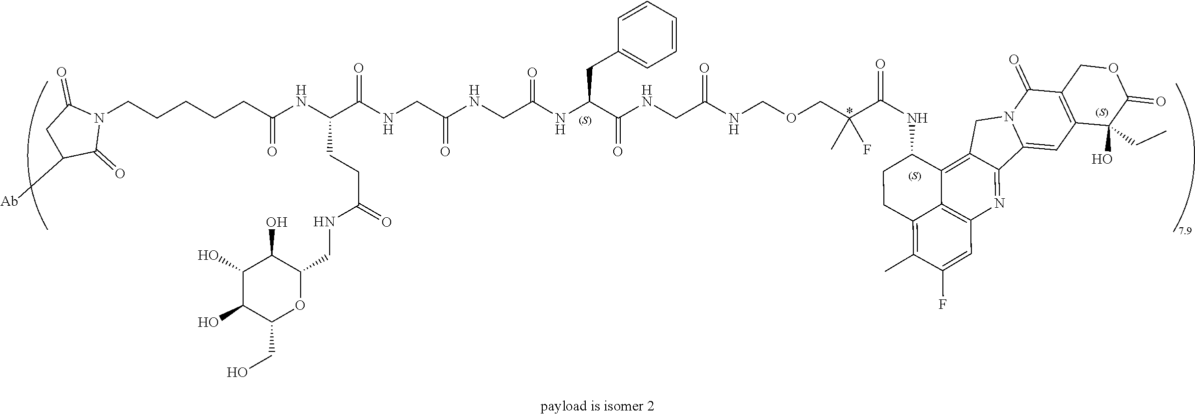 |

PCT Publication No. WO2024/067811A1, the entire contents of which are incorporated herein by reference, discloses antibody drug conjugates, the linker payload portions of which are suitable for use in the context of the present disclosure, and linker payloads which are suitable for use in the context of the present disclosure. In some embodiments, a linker payload is a linker payload disclosed in WO2024/067811A1.

In some embodiments (e.g., of a compound of Formula (I)), P is a residue of Formula (X*):

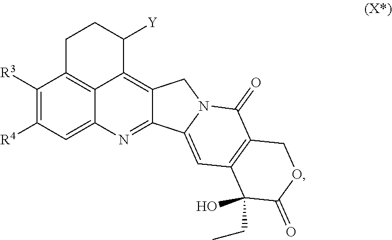

wherein
Y is -A″-B—C″-D′-H;
A″ is a bond, CR¹R², or N—R¹;
B is a bond, —C(═O)—; or —C(═O)O—;
C″ is a bond, or a divalent group, wherein the divalent group is unsubstituted or substituted C₁₋₈alkyl, unsubstituted or substituted cycloalkyl, unsubstituted or substituted heterocyclyl, unsubstituted or substituted aryl, or unsubstituted or substituted heteroaryl;
D′ is a bond, NH, or O;
each of R¹, and R² is, independently, hydrogen, halogen, substituted or unsubstituted alkyl, or substituted or unsubstituted alkoxyl; or R¹, and R² together with the atom to which they are attached to, form unsubstituted or substituted cycloalkyl, unsubstituted or substituted heterocyclyl, unsubstituted or substituted aryl, or unsubstituted or substituted heteroaryl; and
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl. In some embodiments, when R³ is methyl, and R⁴ is F, Y is not —NH—C(═O)—C-D-H.

In embodiments, in the residue of the payload depicted above, Y is -A″-B—C″-D′-, the result of removal of —H from -A″-B—C″-D′-H. It will be appreciated that while a payload residue can result from removal of hydrogen atom from a payload depicted herein, it also can result from removal of a hydroxy group, such as a hydroxy group formed when D′ is O in the payload depicted above (or a corresponding hydroxy group in any of the other payload structures depicted herein).

In some embodiments (e.g., of a compound of Formula (I)), P is a residue of Formula (U*):

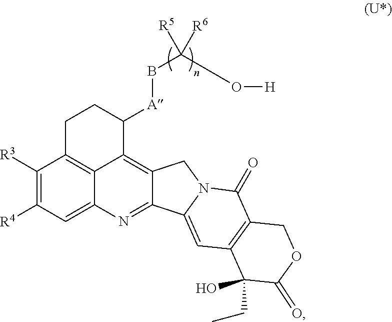

wherein
A″ is CR¹R², NH, or N—R¹;
B is a bond, —C(═O)—; or —C(═O)O—;
each of R¹, and R² is, independently, H, or C₁₋₄alkyl;
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl;
each of R⁵, and R⁶ is, independently, hydrogen, halogen, substituted or unsubstituted alkyl, or substituted or unsubstituted alkoxyl; and
n is 1, 2, 3, 4, or 5.

In some embodiments, A″ is —CH₂—, and B is a bond.

In some embodiments, R⁵ and R⁶ are hydrogen, and n is 1, 2, or 3.

In some embodiments, R³ is methyl, and R⁴ is F.

In some embodiments, P is a residue of:

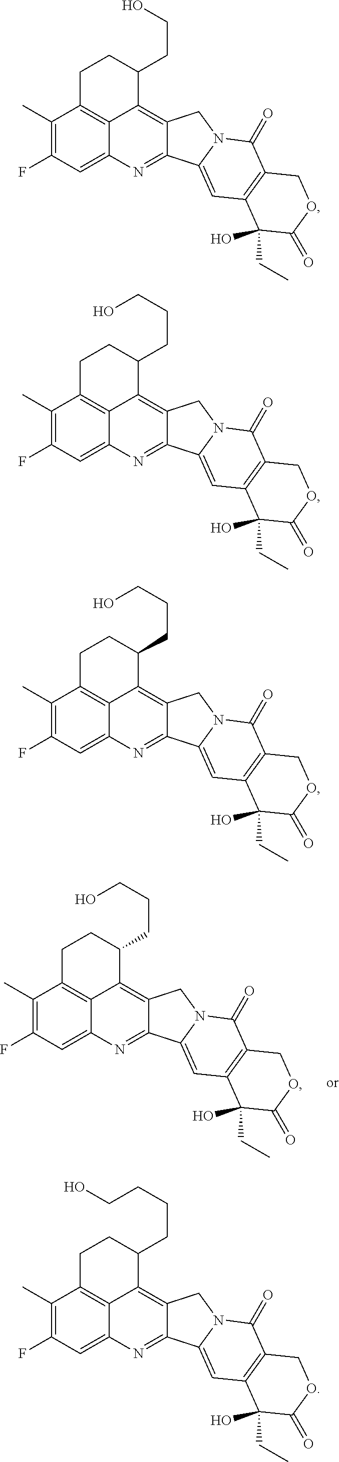 [SMILES: CC[C@@]1(O)C(=O)OCc2c1cc1n(c2=O)Cc2c-1nc1cc(F)c(C)c3c1c2C(CCO)CC3 ; CC[C@@]1(O)C(=O)OCc2c1cc1n(c2=O)Cc2c-1nc1cc(F)c(C)c3c1c2C(CCCO)CC3 ; CC[C@@]1(O)C(=O)OCc2c1cc1n(c2=O)Cc2c-1nc1cc(F)c(C)c3c1c2[C@H](CCCO)CC3 ; CC[C@@]1(O)C(=O)OCc2c1cc1n(c2=O)Cc2c-1nc1cc(F)c(C)c3c1c2[C@@H](CCCO)CC3 ; "or" ; CC[C@@]1(O)C(=O)OCc2c1cc1n(c2=O)Cc2c-1nc1cc(F)c(C)c3c1c2C(CCCCO)CC3]

In some embodiments, R³, and R⁴ together with the atoms to which they are attached, form an unsubstituted or substituted dioxole ring.

In some embodiments, P is a residue of:

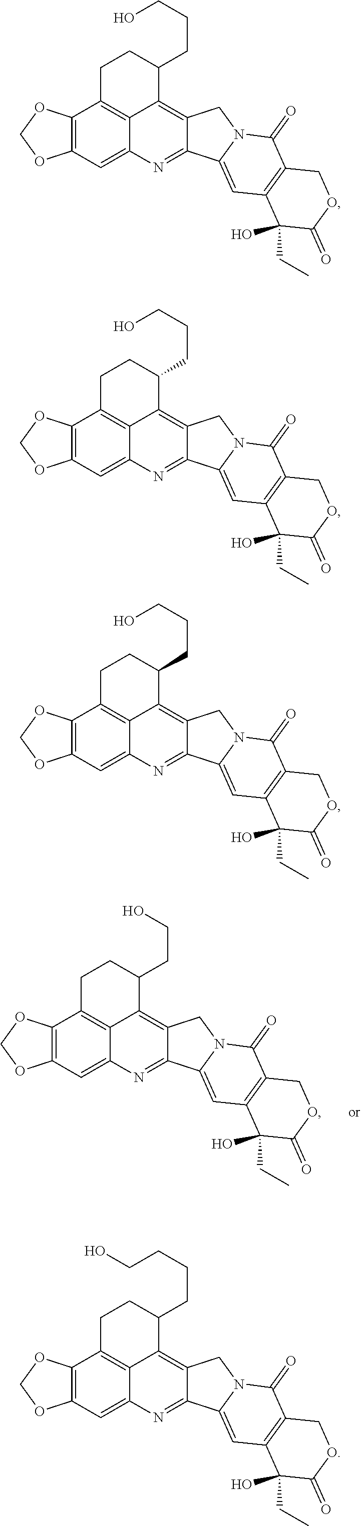 [SMILES: CC[C@@]1(O)C(=O)OCc2c1cc1n(c2=O)Cc2c-1nc1cc3c(c4c1c2C(CCCO)CC4)OCO3 ; CC[C@@]1(O)C(=O)OCc2c1cc1n(c2=O)Cc2c-1nc1cc3c(c4c1c2[C@@H](CCCO)CC4)OCO3 ; CC[C@@]1(O)C(=O)OCc2c1cc1n(c2=O)Cc2c-1nc1cc3c(c4c1c2[C@H](CCCO)CC4)OCO3 ; CC[C@@]1(O)C(=O)OCc2c1cc1n(c2=O)Cc2c-1nc1cc3c(c4c1c2C(CCO)CC4)OCO3 ; "or" ; CC[C@@]1(O)C(=O)OCc2c1cc1n(c2=O)Cc2c-1nc1cc3c(c4c1c2C(CCCCO)CC4)OCO3]

In some embodiments, A″ is —N(CH₃)—, and B is a bond.

In some embodiments, R⁵ and R⁶ are hydrogen, and n is 2.

In some embodiments, P is a residue of:

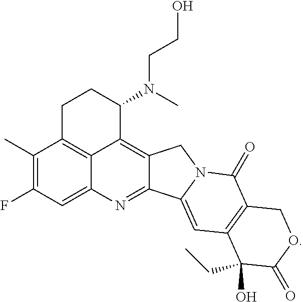 [SMILES: CC[C@@]1(O)C(=O)OCc2c1cc1n(c2=O)Cc2c-1nc1cc(F)c(C)c3c1c2[C@@H](N(C)CCO)CC3]

In some embodiments, A″ is —NH—, and B is —C(═O)O—. In further embodiments, R⁵ and R⁶ are hydrogen, and n is 2.

In some embodiments, P is a residue of:

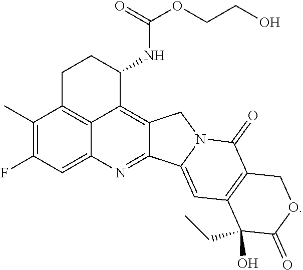 [SMILES: CC[C@@]1(O)C(=O)OCc2c1cc1n(c2=O)Cc2c-1nc1cc(F)c(C)c3c1c2[C@@H](NC(=O)OCCO)CC3]

In some embodiments, A″ is —NH—, and B is —C(═O)—. In further embodiments, R and R⁶ are hydrogen, and n is 2.

In some embodiments, P is a residue of

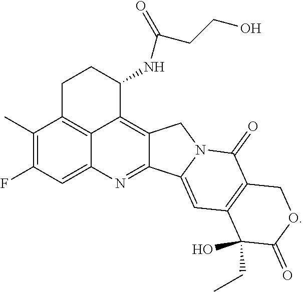 [SMILES: CC[C@@]1(O)C(=O)OCc2c1cc1n(c2=O)Cc2c-1nc1cc(F)c(C)c3c1c2[C@@H](NC(=O)CCO)CC3]

In some embodiments, R³ is Cl, R⁴ is F, and B is —C(═O)—.

In some embodiments, P is a residue of

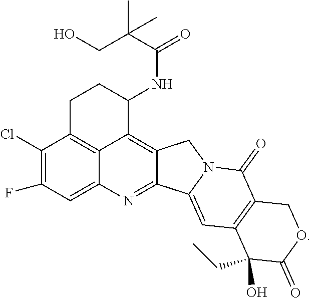 [SMILES: CC[C@@]1(O)C(=O)OCc2c1cc1n(c2=O)Cc2c-1nc1cc(F)c(Cl)c3c1c2C(NC(=O)C(C)(C)CO)CC3]

In some embodiments, R³ is methyl, R⁴ is Cl, and B is —C(═O)—.

In some embodiments, P is a residue of

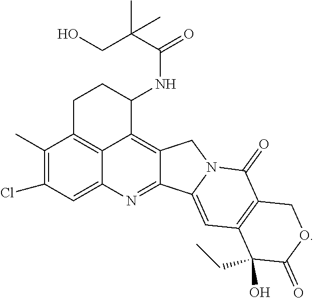 [SMILES: CC[C@@]1(O)C(=O)OCc2c1cc1n(c2=O)Cc2c-1nc1cc(Cl)c(C)c3c1c2C(NC(=O)C(C)(C)CO)CC3]

In some embodiments, R³, and R⁴ together with the atoms to which they are attached to, form unsubstituted or substituted heterocyclyl.

In some embodiments, R³, and R⁴ together with the atoms to which they are attached to, form an unsubstituted or substituted dioxole ring, and B is —C(═O)—.

In some embodiments, P is a residue of:

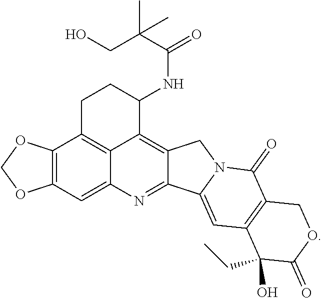 [SMILES: CC[C@@]1(O)C(=O)OCc2c1cc1n(c2=O)Cc2c-1nc1cc3c(c4c1c2C(NC(=O)C(C)(C)CO)CC4)OCO3]

In some embodiments, P is a residue of:

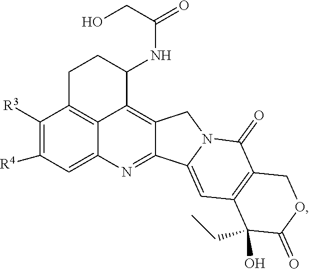

wherein values and alternative values for the variables (e.g., R³, R⁴) are as described elsewhere herein, for example, with respect to compounds of Formula (X*) and (U*).

In some embodiments, R³ is methyl; and R⁴ is Cl.

In some embodiments, P is a residue of:

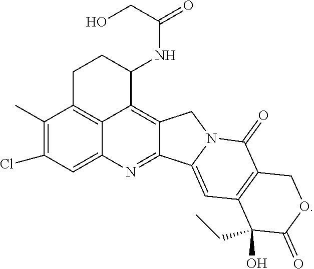 [SMILES: CC[C@@]1(O)C(=O)OCc2c1cc1n(c2=O)Cc2c-1nc1cc(Cl)c(C)c3c1c2C(NC(=O)CO)CC3]

In some embodiments, R³ is Cl; and R⁴ is F.

In some embodiments, P is a residue of:

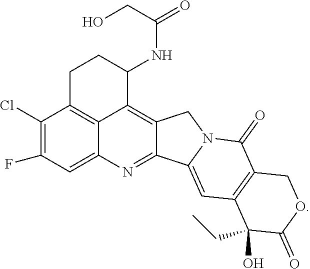 [SMILES: CC[C@@]1(O)C(=O)OCc2c1cc1n(c2=O)Cc2c-1nc1cc(F)c(Cl)c3c1c2C(NC(=O)CO)CC3]

In some embodiments, R³ is F; and R⁴ is F.

In some embodiments, P is a residue of:

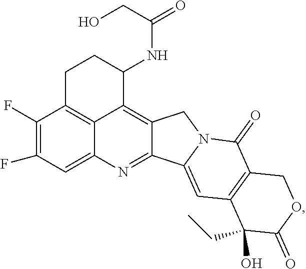 [SMILES: CC[C@@]1(O)C(=O)OCc2c1cc1n(c2=O)Cc2c-1nc1cc(F)c(F)c3c1c2C(NC(=O)CO)CC3]

In some embodiments, R³ is H; and R⁴ is F.

In some embodiments, P is a residue of

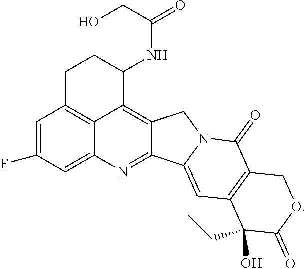 [SMILES: CC[C@@]1(O)C(=O)OCc2c1cc1n(c2=O)Cc2c-1nc1cc(F)cc3c1c2C(NC(=O)CO)CC3]

In some embodiments, R³ is H; and R⁴ is OH.

In some embodiments, P is a residue of:

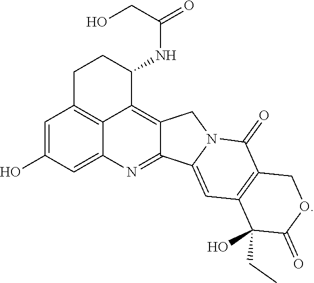 [SMILES: CC[C@@]1(O)C(=O)OCc2c1cc1n(c2=O)Cc2c-1nc1cc(O)cc3c1c2[C@@H](NC(=O)CO)CC3]

In some embodiments, R³ is methyl; and R⁴ is methyl.

In some embodiments, P is a residue of:

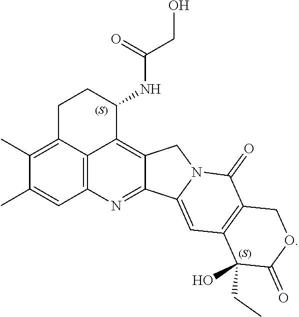

In some embodiments, R³ is methoxyl; and R⁴ is F.

In some embodiments, P is a residue of:

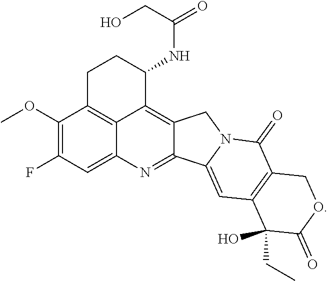 [SMILES: CC[C@@]1(O)C(=O)OCc2c1cc1n(c2=O)Cc2c-1nc1cc(F)c(OC)c3c1c2[C@@H](NC(=O)CO)CC3]

In some embodiments, R³ is H; and R⁴ is methoxyl.

In some embodiments, P is a residue of:

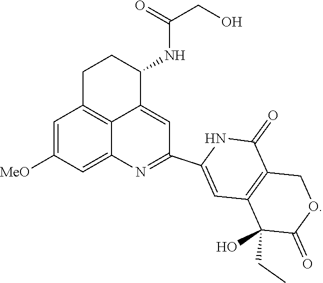 [SMILES: CC[C@@]1(O)C(=O)OCc2c1cc(-c1cc3c4c(cc(OC)cc4n1)CC[C@@H]3NC(=O)CO)[nH]c2=O]

In some embodiments, R³ is H; and R⁴ is Cl.

In some embodiments, P is a residue of:

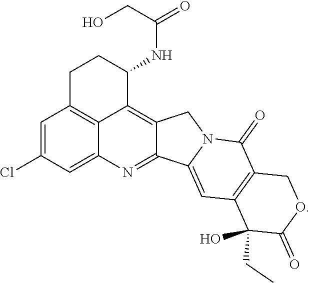 [SMILES: CC[C@@]1(O)C(=O)OCc2c1cc1n(c2=O)Cc2c-1nc1cc(Cl)cc3c1c2[C@@H](NC(=O)CO)CC3]

In some embodiments, R³ and R⁴ together with the atoms to which they are attached to, form unsubstituted or substituted heterocyclyl.

In some embodiments, R³, and R⁴ together with the atoms to which they are attached to, form an unsubstituted or substituted dioxole ring.

In some embodiments, P is a residue of:

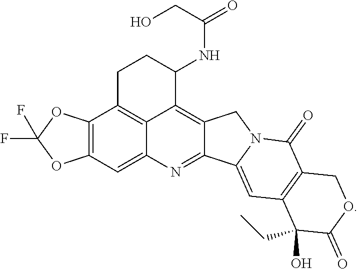 [SMILES: CC[C@@]1(O)C(=O)OCc2c1cc1n(c2=O)Cc2c-1nc1cc3c(c4c1c2C(NC(=O)CO)CC4)OC(F)(F)O3]

In some embodiments, P is a residue of:

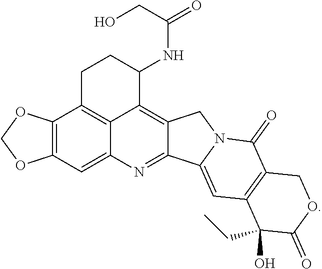 [SMILES: CC[C@@]1(O)C(=O)OCc2c1cc1n(c2=O)Cc2c-1nc1cc3c(c4c1c2C(NC(=O)CO)CC4)OCO3]

In some embodiments, P is a residue of:

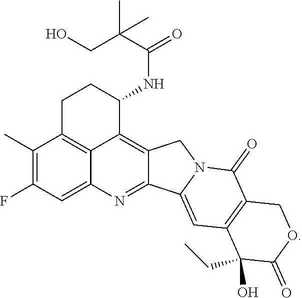 [SMILES: CC[C@@]1(O)C(=O)OCc2c1cc1n(c2=O)Cc2c-1nc1cc(F)c(C)c3c1c2[C@@H](NC(=O)C(C)(C)CO)CC3]

In some embodiments, P is a residue of:

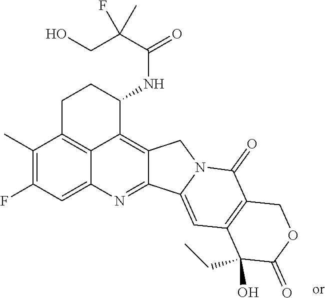 [SMILES: CC[C@@]1(O)C(=O)OCc2c1cc1n(c2=O)Cc2c-1nc1cc(F)c(C)c3c1c2[C@@H](NC(=O)C(C)(F)CO)CC3]

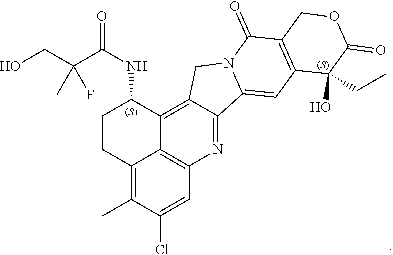

In some embodiments (e.g., of a compound of Formula (I)), P is a residue of:

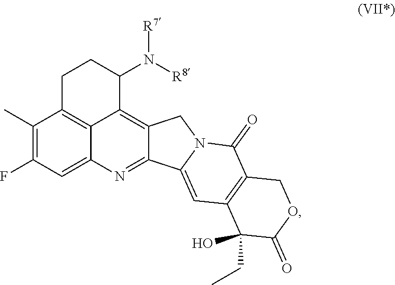

wherein
each of R⁷, and R^{8′} is, independently, hydrogen, or substituted or unsubstituted alkyl; or R^{7′}, and R^{8′} together with the nitrogen atom to which they are attached to, form unsubstituted or substituted heterocyclyl, or unsubstituted or substituted heteroaryl.

In some embodiments, P is a residue of:

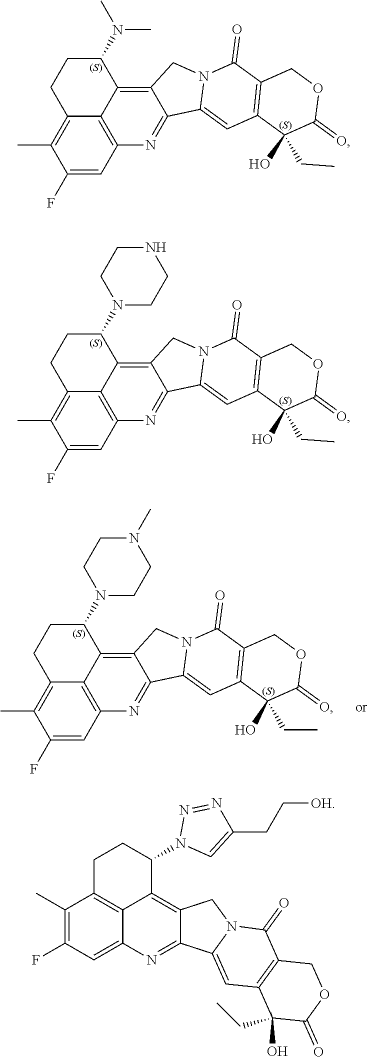

In embodiments, P has formula E1*:

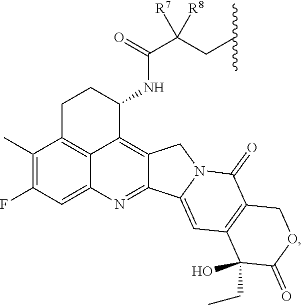

wherein R⁷ and R⁸ are as described elsewhere herein, e.g., for formula E1*.

In embodiments, P has the following formula:

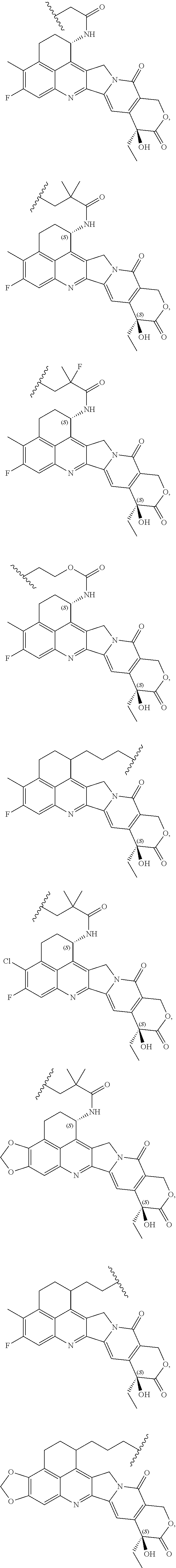

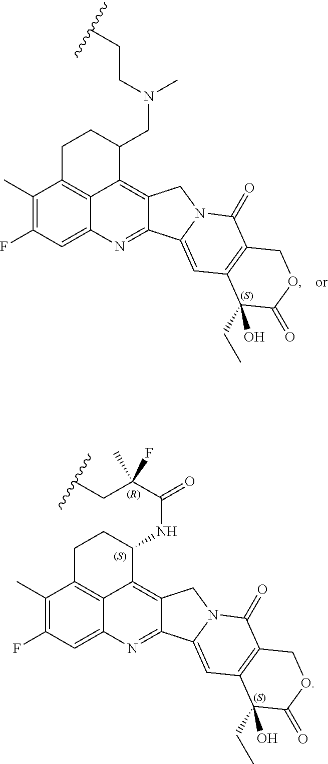

In certain embodiments, P is

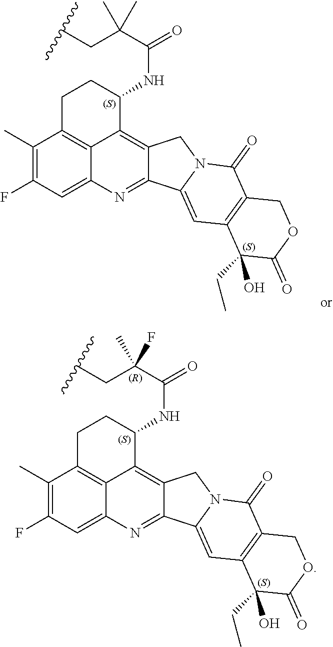

In certain embodiments, P is

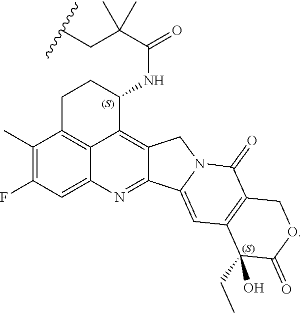

The cytotoxic agent may be directly joined to an antibody disclosed herein, or indirectly joined to an antibody disclosed herein, via a linker (L). In embodiments, the linker is cleavable, such as by an enzyme, to release the cytotoxic agent. In embodiments, such as when the cytotoxic agent is hydrophobic, the linker is hydrophilic. In embodiments, the linker has the following formula, in which * marks the bond where L may be joined to a conjugator (C):

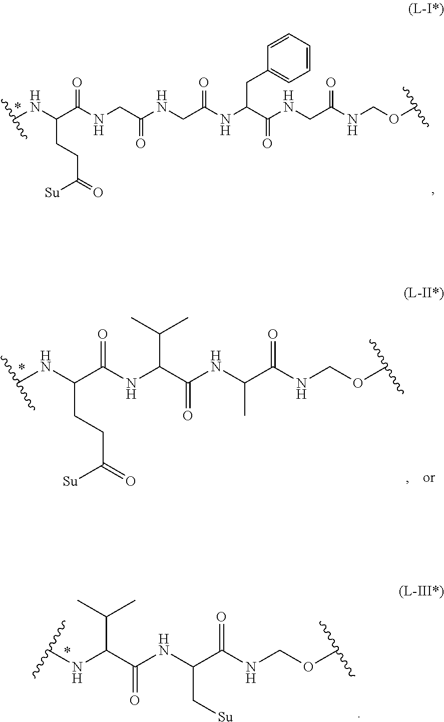

In the foregoing formulae L-I*, L-II*, and L-III*, Su may be a sugar-like moiety. The moiety may be derived from a natural or non-natural sugar. The moiety may be hydrophilic. In embodiments, such as when the cytotoxic agent is hydrophobic, inclusion of a hydrophilic Su moiety may reduce the likelihood of antibody drug conjugate aggregation and thereby reduce clearance rate in vivo.

In embodiments, Su is a hydrophilic residue.

In embodiments, Su is

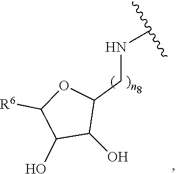

wherein n8 is 0 or 1; R⁶ is —OR², —N(H)R², —C(O)OR², —C(O)N(H)R², —CH₂—OR², —CH₂—N(H)R², —CH₂—C(O)OR², or —CH₂—C(O)N(H)R²; and R² is hydrogen or methyl. In certain embodiments, n8 is 1. In certain embodiments, n8 is 0. In certain embodiments, R² is hydrogen. In certain embodiments, R² is methyl. In certain embodiments, R⁶ is —OR², —N(H)R², —C(O)OR² or —C(O)N(H)R². In certain embodiments R⁶ is —CH₂—OR², —CH₂—N(H)R², —CH₂—C(O)OR², or —CH₂—C(O)N(H)R². In certain embodiments, R⁶ is —CH₂—C(O)N(H)R², e.g., —CH₂—C(O)NH₂.

In embodiments, Su is

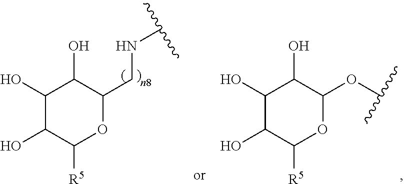

wherein n8 is 0 or 1; R⁵ is —OH, —NH₂, —C(O)OH, —C(O)NH₂, —CH₂—OH, or —CH₂—NH₂. In certain embodiments, Su is

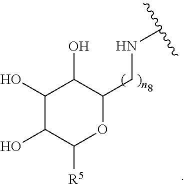

In certain embodiments, Su is

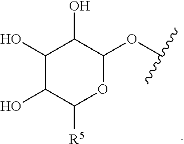

In certain embodiments, n8 is 0. In certain embodiments, n8 is 1. In certain embodiments, R⁵ is —CH₂OH. In certain embodiments, R⁵ is —C(O)OH.

In embodiments, Su is

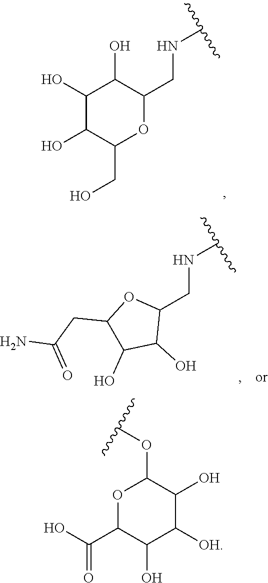 [SMILES: *NCC1OC(CO)C(O)C(O)C1O ; *NCC1OC(CC(N)=O)C(O)C1O ; *OC1OC(C(=O)O)C(O)C(O)C1O]

In certain embodiments, Su is

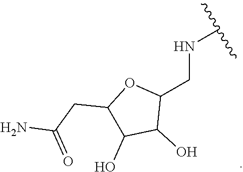 [SMILES: *NCC1OC(CC(N)=O)C(O)C1O]

In embodiments, Su is

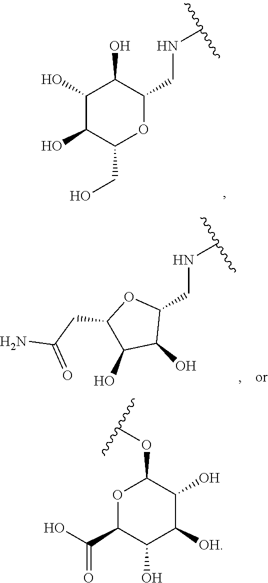 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

In certain embodiments, Su is

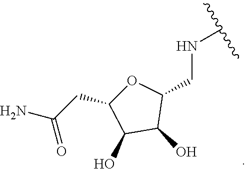 [SMILES: *NC[C@H]1O[C@@H](CC(N)=O)[C@H](O)[C@@H]1O]

In embodiments, Su is:

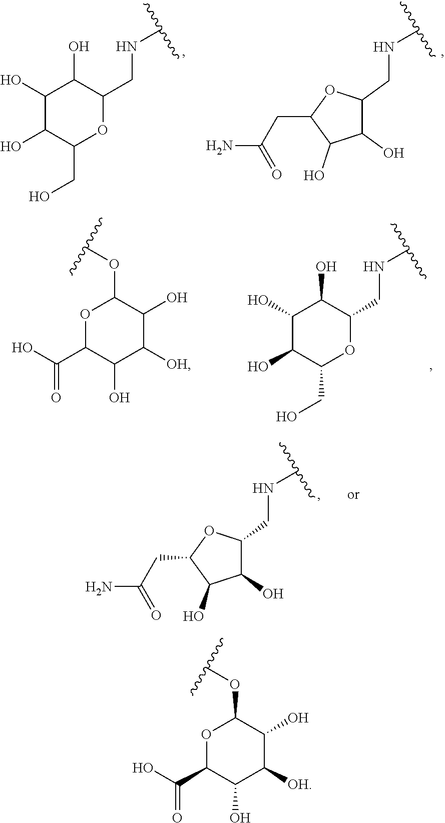 [SMILES: *NCC1OC(CO)C(O)C(O)C1O ; *NCC1OC(CC(N)=O)C(O)C1O ; *OC1OC(C(=O)O)C(O)C(O)C1O ; *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

In embodiments, L is

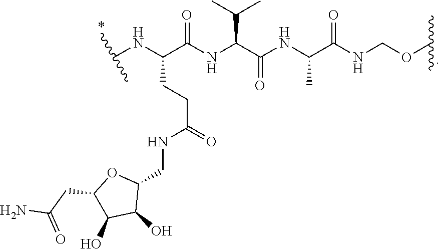 [SMILES: *N[C@@H](CCC(=O)NC[C@H]1O[C@@H](CC(N)=O)[C@H](O)[C@@H]1O)C(=O)N[C@H](C(=O)N[C@@H](C)C(=O)NCO*)C(C)C]

The antibody drug conjugates disclosed herein may include a conjugator (C). The conjugator may be indirectly joined to the cytotoxic agent via the linker. The conjugator may help avoid or reduce deconjugation of the cytotoxic agent in vivo, which may help maintain a stable drug-to-antibody ratio (DAR). The conjugator, e.g., prior to conjugation with an antibody, may have the following formula:

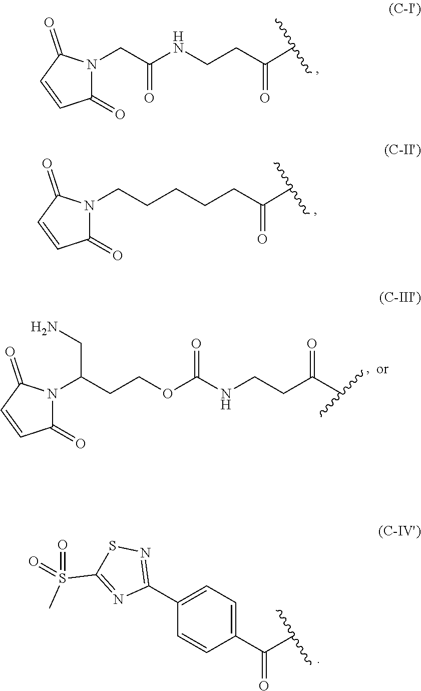 [SMILES: *C(=O)CCNC(=O)CN1C(=O)C=CC1=O ; *C(=O)CCCCCN1C(=O)C=CC1=O ; *C(=O)CCNC(=O)OCCC(CN)N1C(=O)C=CC1=O ; *C(=O)c1ccc(-c2nsc(S(C)(=O)=O)n2)cc1]

When the conjugator is joined directly to an antibody disclosed herein, the conjugator may have the following formula, in which * marks the bond where the conjugator connects to the antibody:

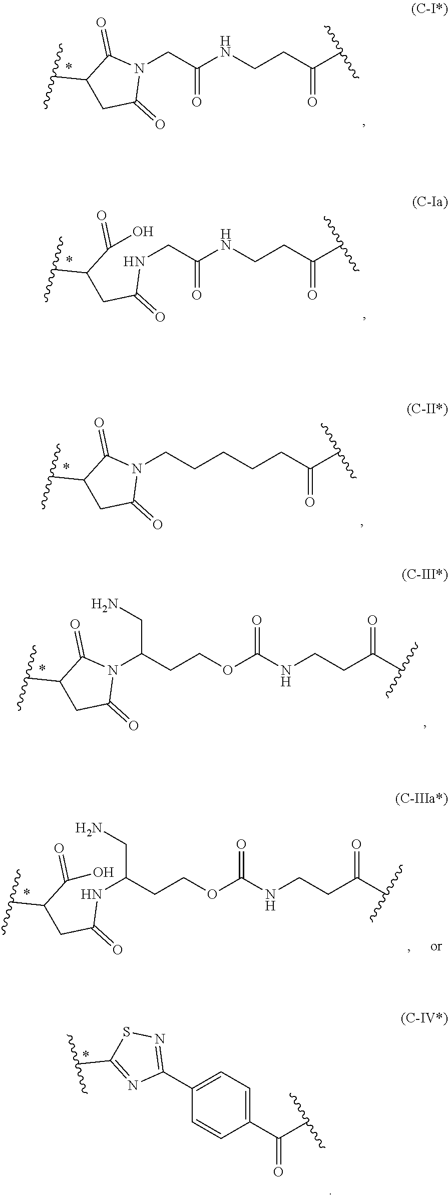 [SMILES: *C(=O)CCNC(=O)CN1C(=O)CC(*)C1=O ; *C(=O)CCNC(=O)CNC(=O)CC(*)C(=O)O ; *C(=O)CCCCCN1C(=O)CC(*)C1=O ; *C(=O)CCNC(=O)OCCC(CN)N1C(=O)CC(*)C1=O ; *C(=O)CCNC(=O)OCCC(CN)NC(=O)CC(*)C(=O)O ; *C(=O)c1ccc(-c2nsc(*)n2)cc1]

The conjugator, linker, sugar moiety, and cytotoxic agent, may be included in the presently disclosed antibody drug conjugates in any combination. Non-limiting examples of conjugator (pre-conjugation)-linker-cytotoxic agent combinations include the following:

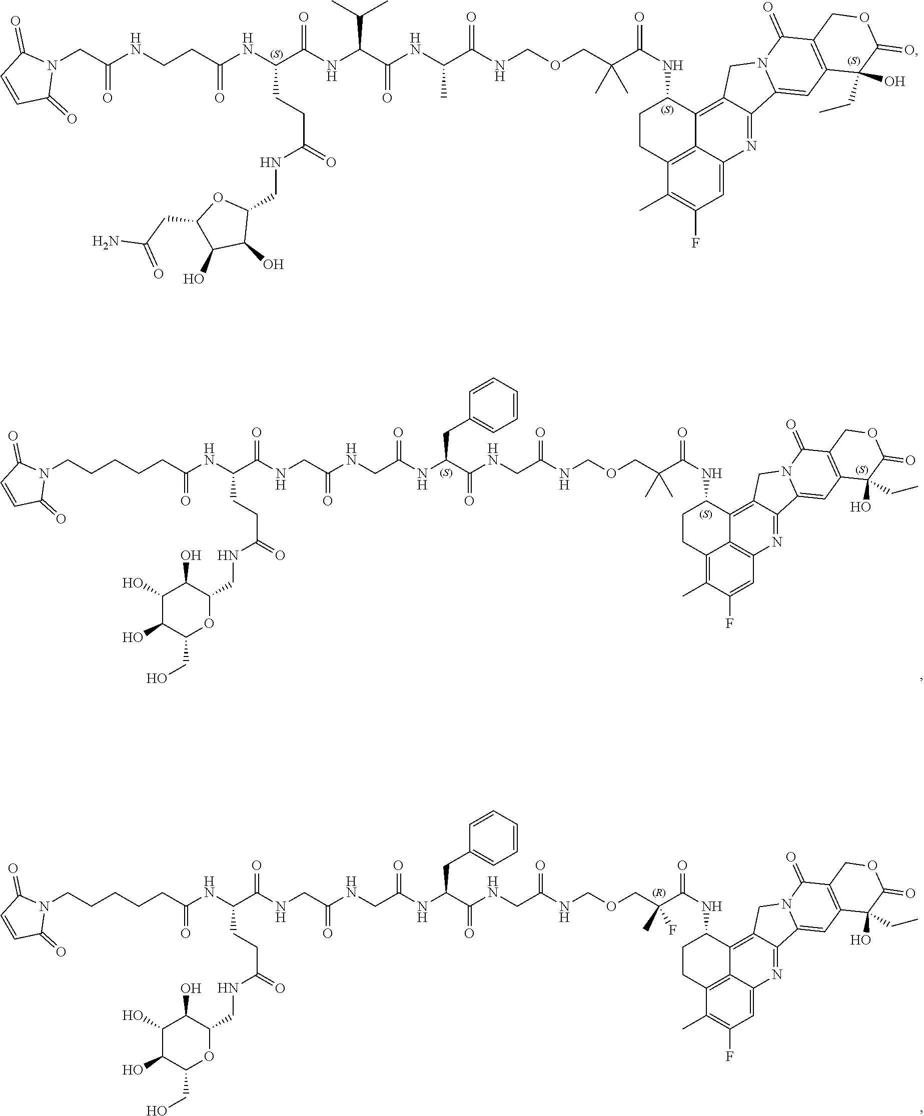

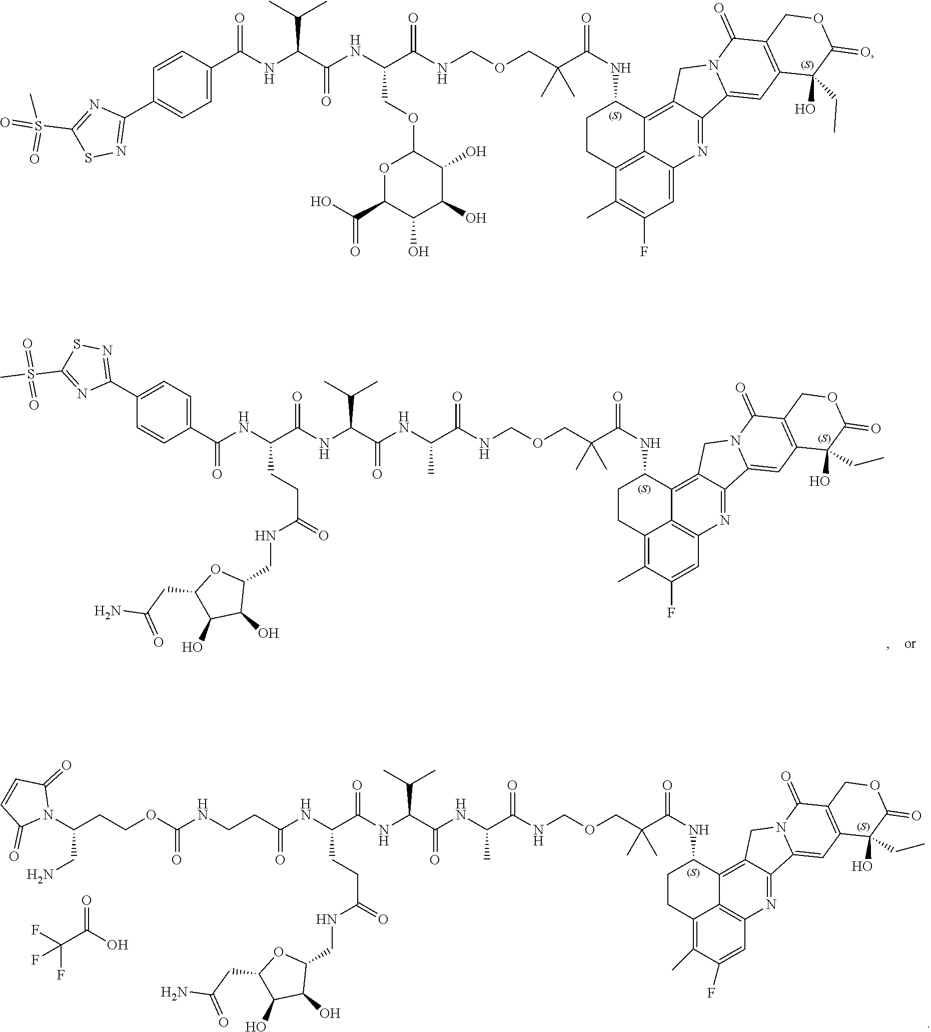

Each antibody drug conjugate may include more than one compound of conjugator-linker-cytotoxic agent (C-L-D), such as one, two, three, four, five, six, seven, eight, nine, or ten C-L-D. In embodiments, each antibody drug conjugate includes from 1 to 10, e.g., from 3 to 10, from 4 to 10, from 5 to 10, from 6 to 10, from 7 to 9, or about 8.

In certain embodiments, —C-L-(D)ₘ is:

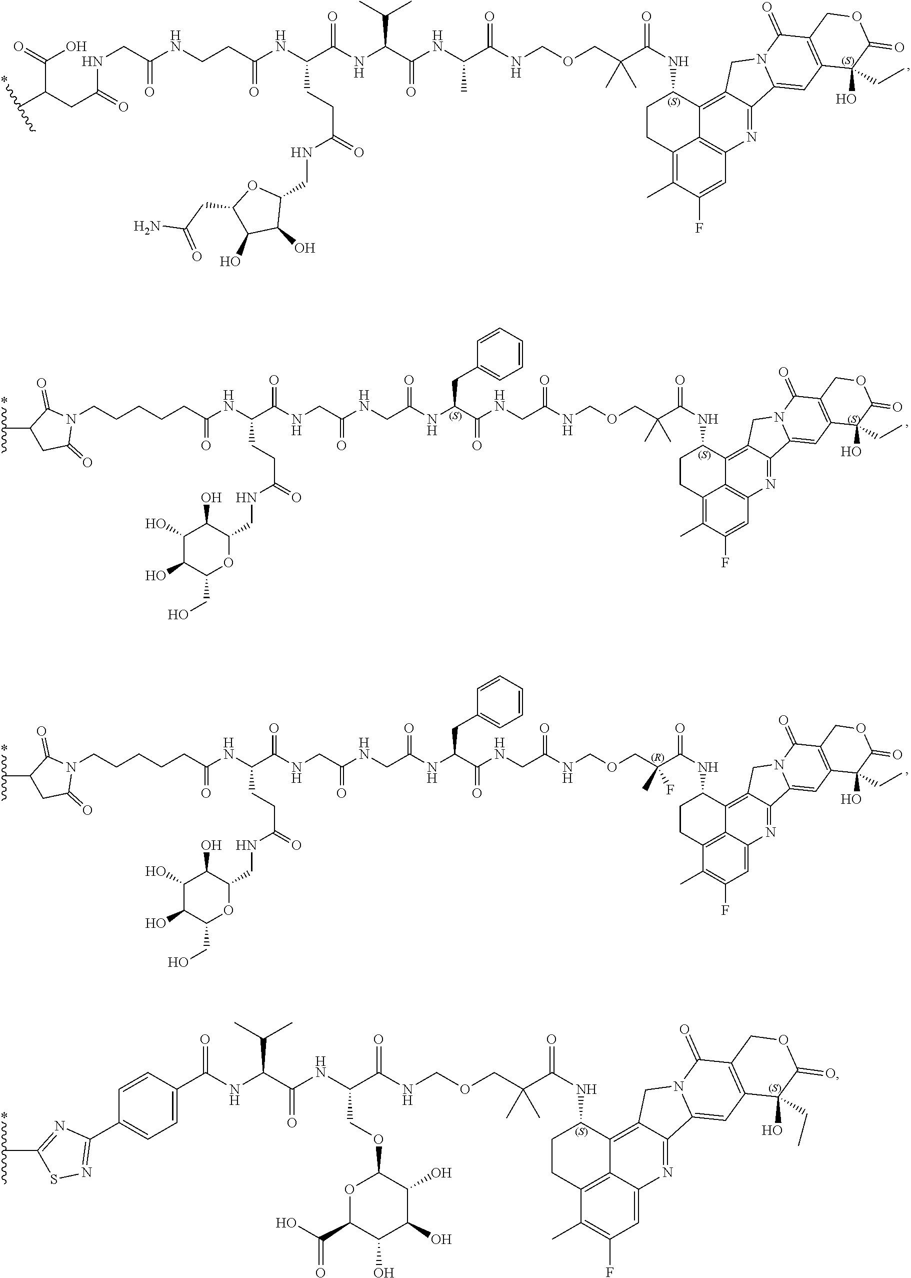

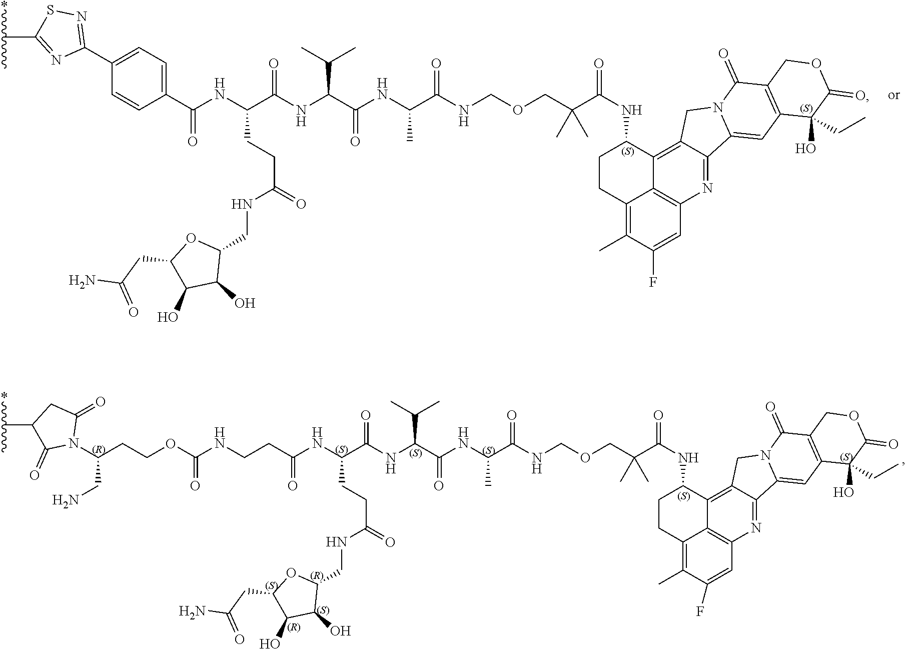

where * marks the bond where C connects to Ab. In certain embodiments, —C-L-(D)ₘ is:

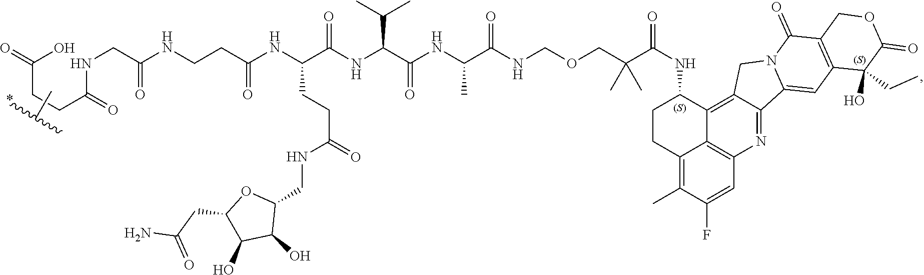

where * marks the bond where C connects to Ab. In certain embodiments, —C-L-(D)ₘ is:

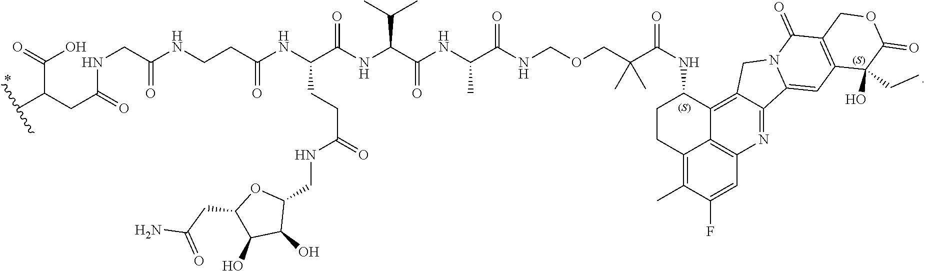

In certain embodiments, —C-L-(D)ₘ is:

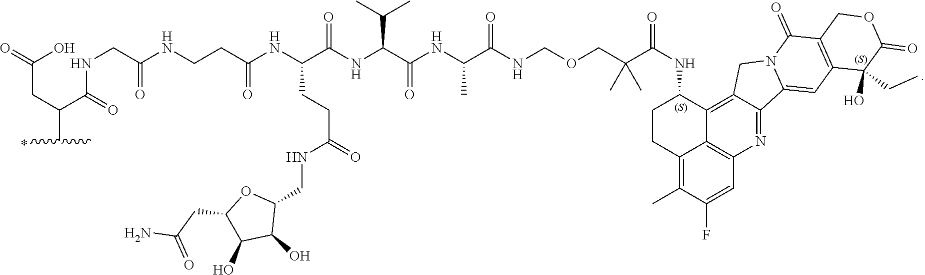

In embodiments, the antibody drug conjugate is

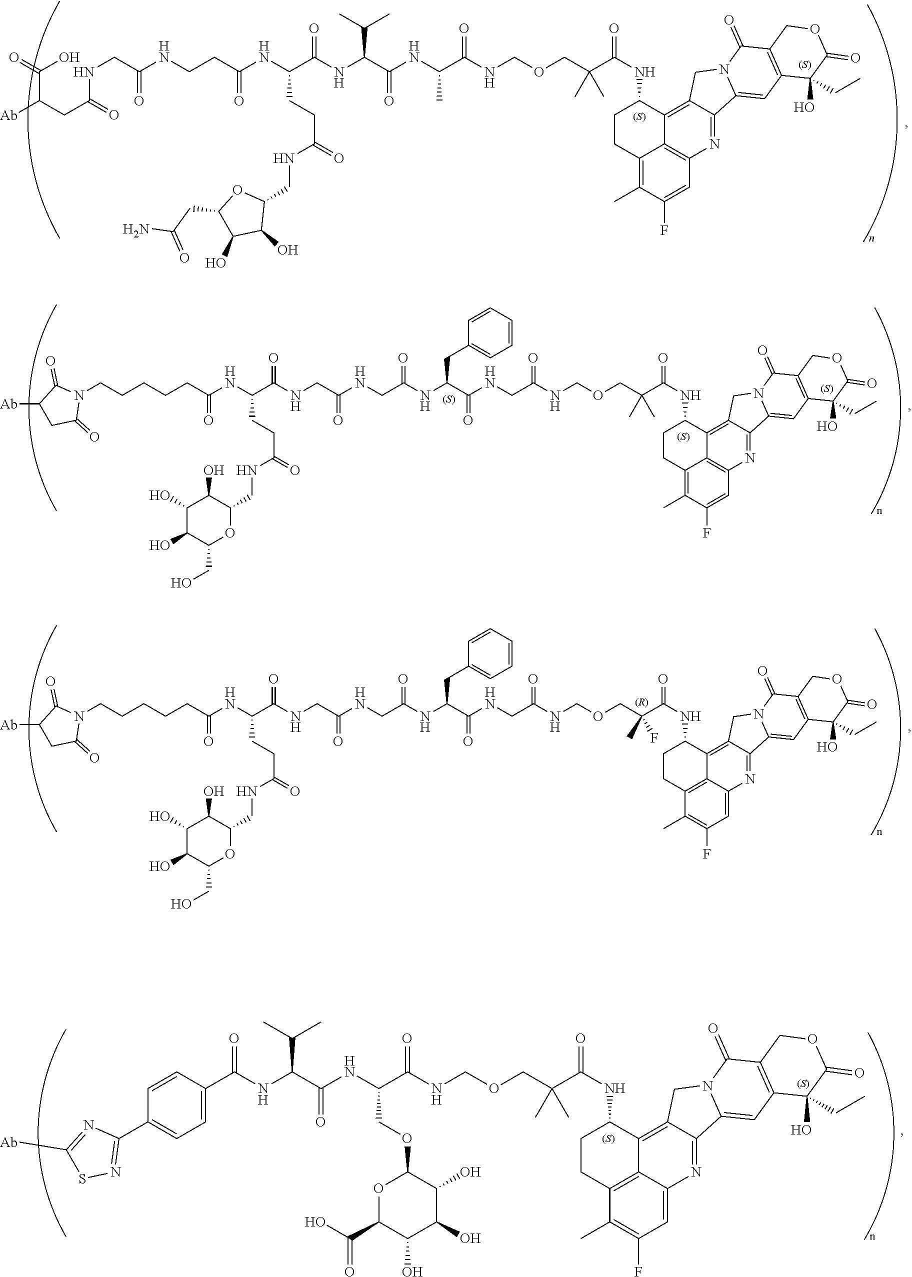

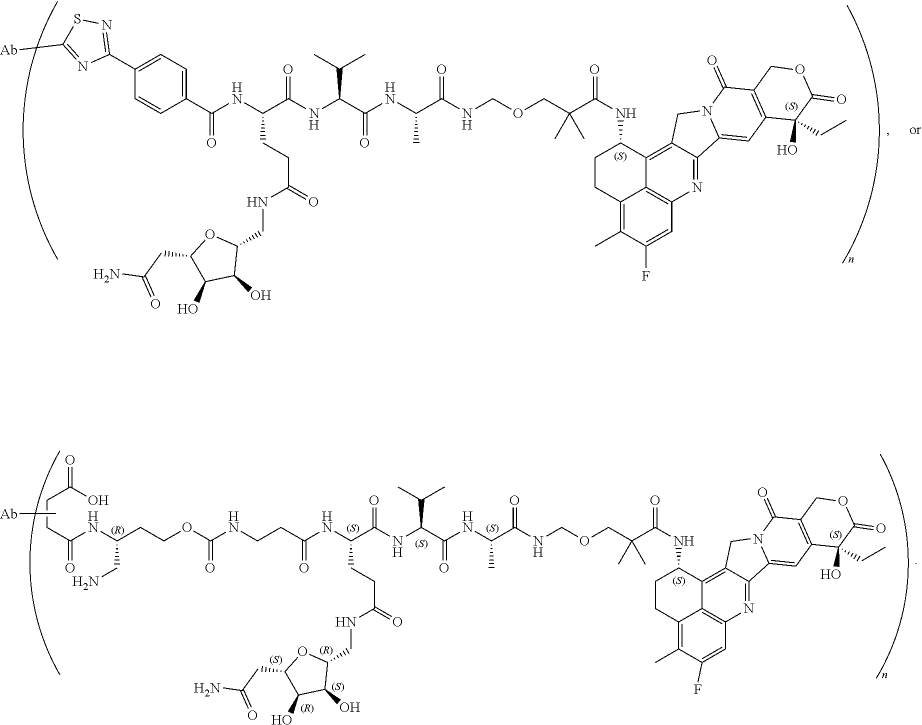

or a tautomer, pharmaceutically acceptable salt, solvate or hydrate thereof, wherein Ab and n are as described herein.

In certain embodiments, the antibody drug conjugate is of the following formula:

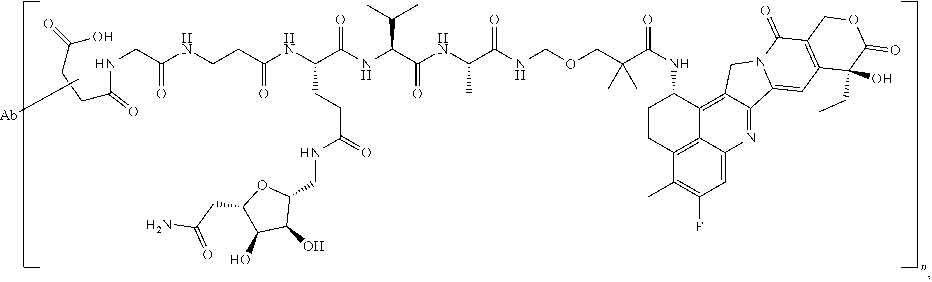

or a tautomer, pharmaceutically acceptable salt, solvate or hydrate thereof, wherein Ab and n are as described herein. In certain embodiments, the antibody drug conjugate is of the following formula:

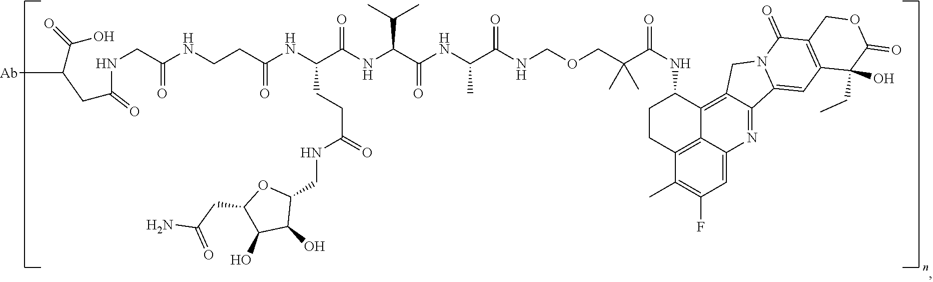

or a tautomer, pharmaceutically acceptable salt, solvate or hydrate thereof, wherein Ab and n are as described herein. In certain embodiments, the antibody drug conjugate is of the following formula:

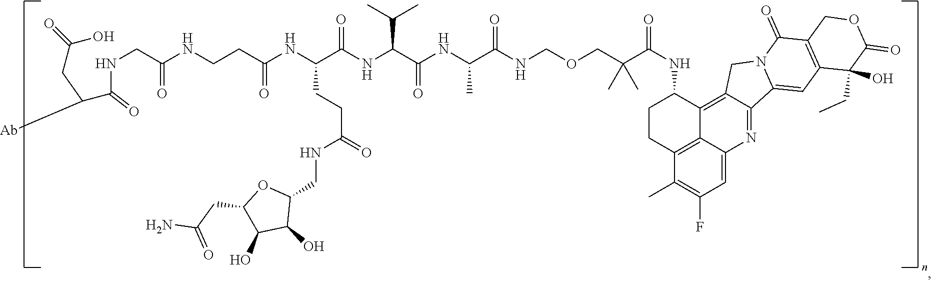

or a tautomer, pharmaceutically acceptable salt, solvate or hydrate thereof, wherein Ab and n are as described herein.

In some embodiments, an antibody drug conjugate has formula (V*):

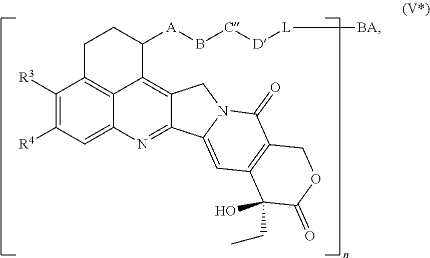

or a pharmaceutically acceptable salt tautomer, solvate, stereoisomer, enantiomer, isotopologue, or prodrug thereof, wherein values and alternative values for the variables (e.g., A, B, C″, D′, L, R³, R⁴, and n) are as described elsewhere herein, for example, with respect to compounds of Formula (X*) and (U*).

In some embodiments, an antibody drug conjugate has a structure of Formula (VIIIa*), (VIIIb*), or (VIIIc*):

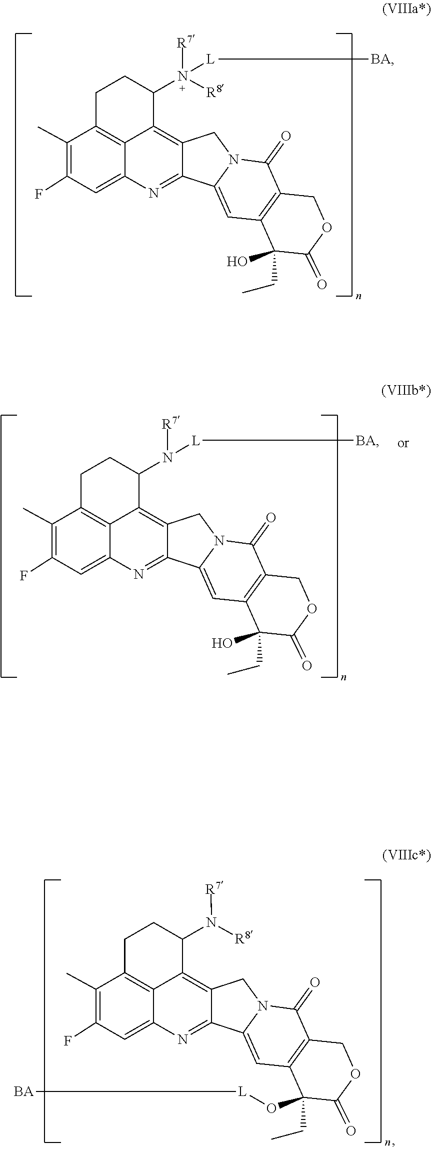

or a pharmaceutically acceptable salt tautomer, solvate, stereoisomer, enantiomer, isotopologue, or prodrug thereof, wherein values and alternative values for the variables (e.g., L, R⁷, R⁸, and n) are as described elsewhere herein, for example, in respect to compounds of Formula (X*) or (U*).

In some embodiments, an antibody drug conjugate has a structure of any one of the following formulas:

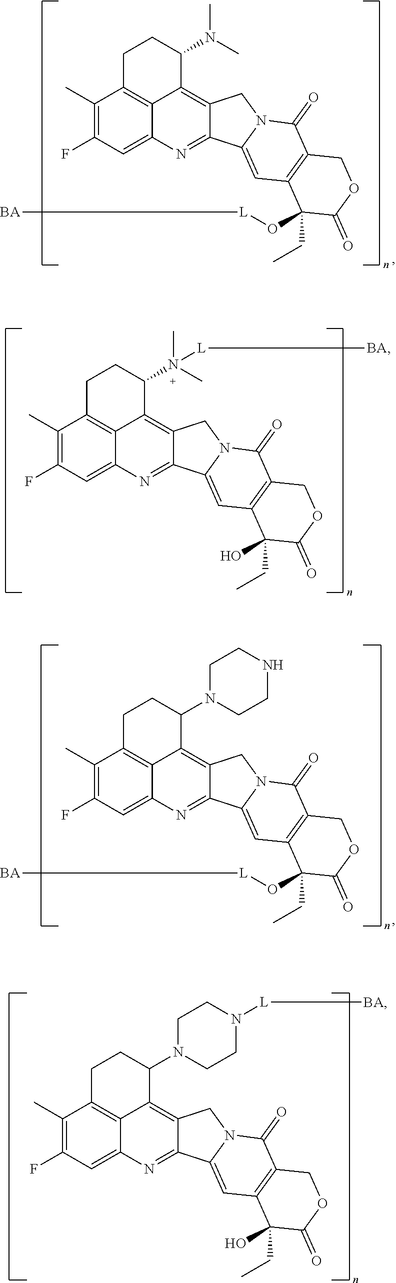

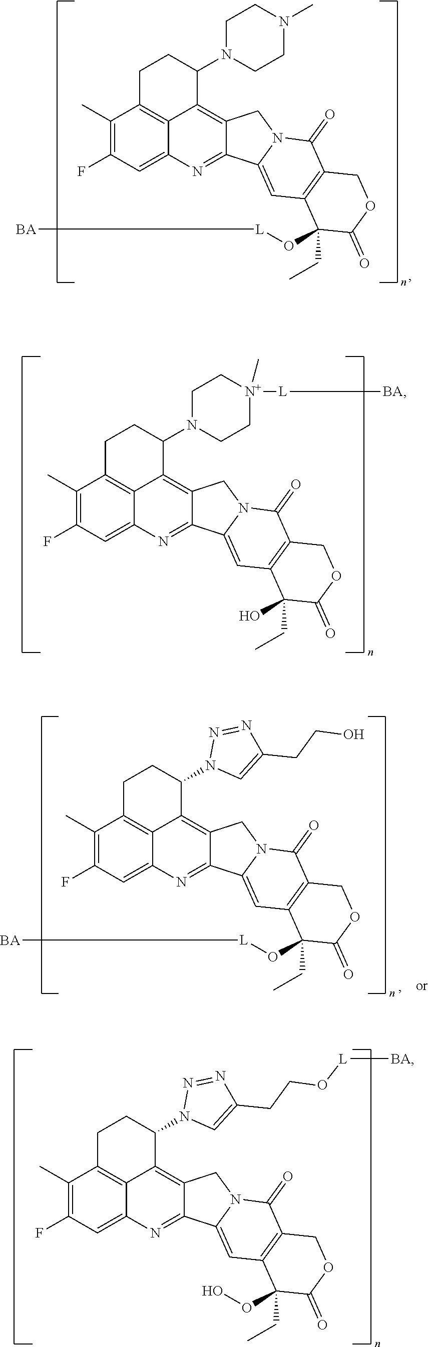

or a pharmaceutically acceptable salt tautomer, solvate, stereoisomer, enantiomer, isotopologue, or prodrug thereof, wherein values and alternative values for the variables (e.g., L and n) are as described elsewhere herein.

In some embodiments, L is

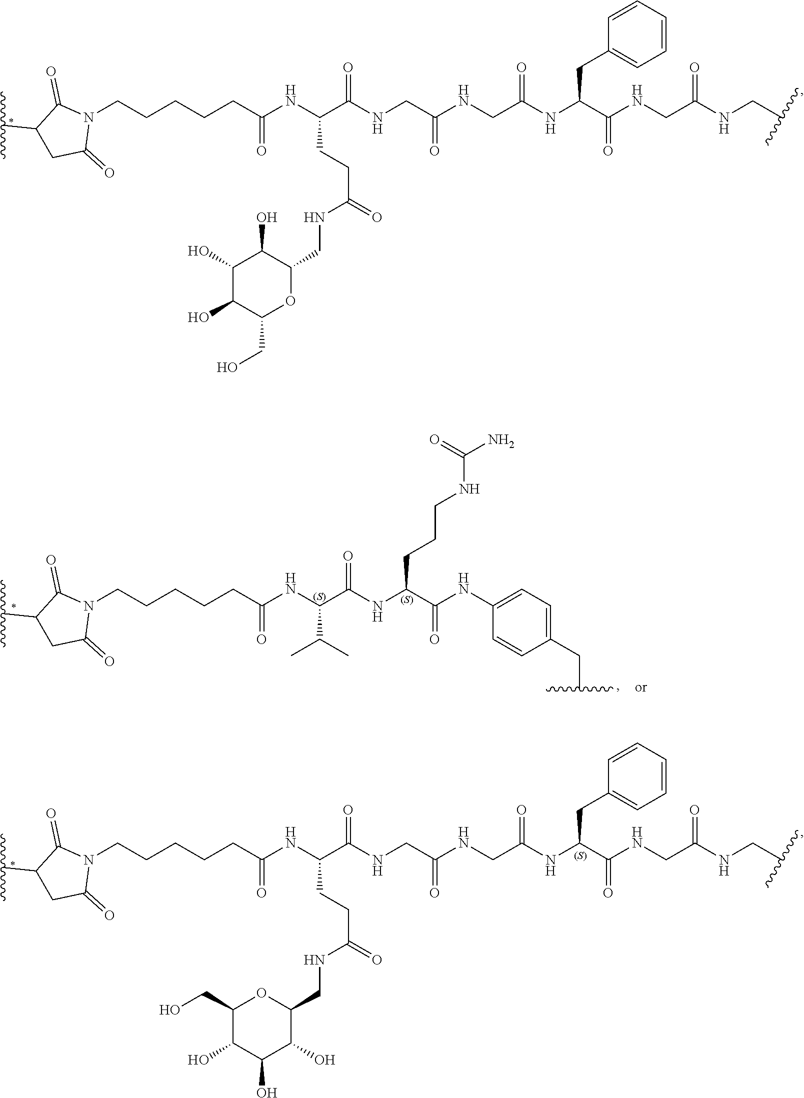

wherein the bond marked with asterisk is connected to BA.

In some embodiments, Lis

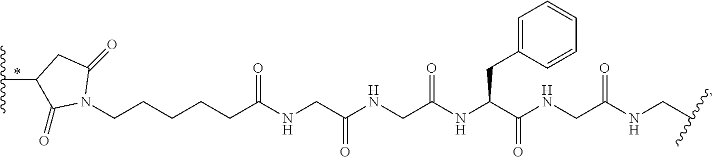 [SMILES: *CNC(=O)CNC(=O)[C@H](Cc1ccccc1)NC(=O)CNC(=O)CNC(=O)CCCCCN1C(=O)CC(*)C1=O]

wherein the bond marked with asterisk is connected to BA.

In some embodiments, L is:

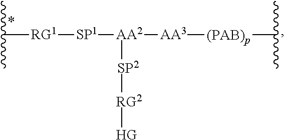

wherein values and alternative values for the variables (e.g., RG¹, SP¹, AA², AA³, PAB, p, SP² RG², and HG¹) are as described herein, for example, with respect to compounds of Formulas (Ia*), (Ib*), and (Ic*).

In some embodiments, L is:

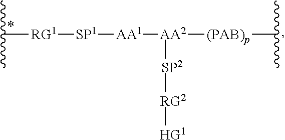

wherein values and alternative values for the variables (e.g., RG¹, SP¹, AA¹, AA², PAB, p, SP² RG², and HG¹) are as described elsewhere herein, for example, with respect to compounds of Formulas (Ia*), (Ib*), and (Ic*).

In some embodiments, L is:

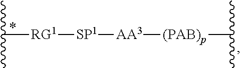

wherein values and alternative values for the variables (e.g., RG¹, SP¹, AA³, PAB, and p) are as described elsewhere herein, for example, with respect to compounds of Formulas (Ia*), (Ib*), and (Ic*).

In some embodiments, -AA²(SP²—RG²-HG¹)-AA³-(PAB)ₚ- is

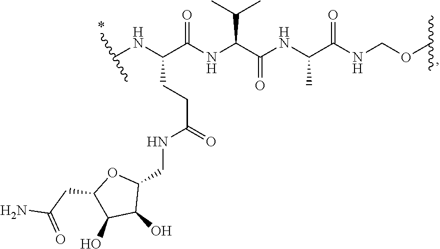 [SMILES: *N[C@@H](CCC(=O)NC[C@H]1O[C@@H](CC(N)=O)[C@H](O)[C@@H]1O)C(=O)N[C@H](C(=O)N[C@@H](C)C(=O)NCO*)C(C)C]

wherein * marks the bond that connects to SP¹.

In some embodiments, an antibody drug conjugate is selected from the following, or a pharmaceutically acceptable salt tautomer, solvate, stereoisomer, enantiomer, isotopologue, or prodrug thereof, wherein Ab is any of the antibodies or antigen-binding fragments thereof as disclosed herein:

| Ex- | |
|---|---|
| ample | |
| No. | Antibody Drug Conjugate Structure |
| ADC.P1 | 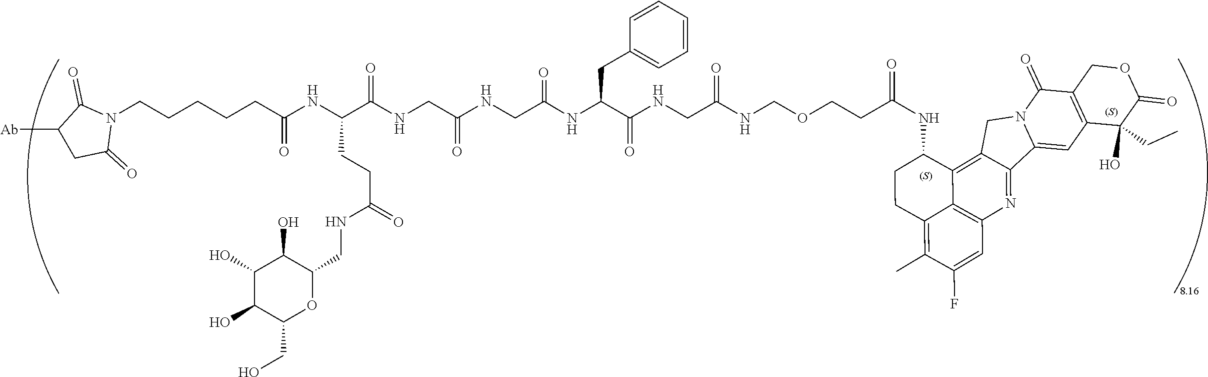 |
| ADC.P2 | 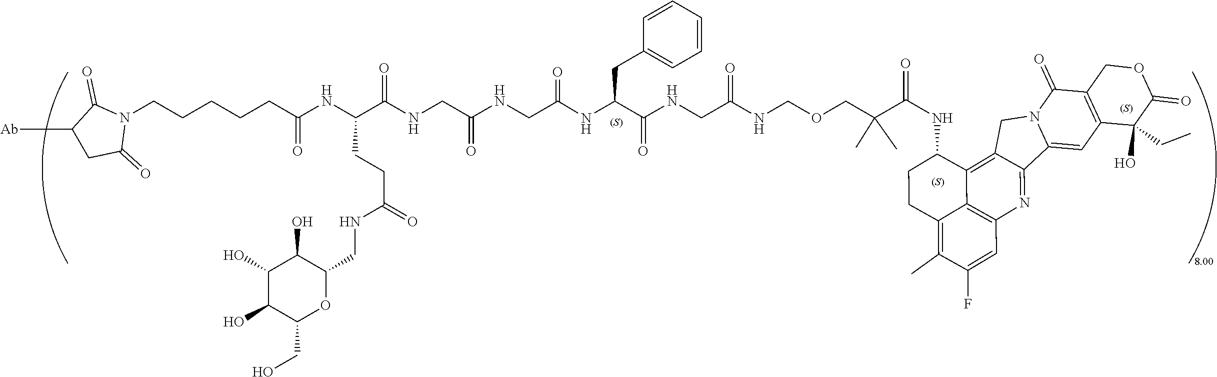 |
| ADC.P3 | 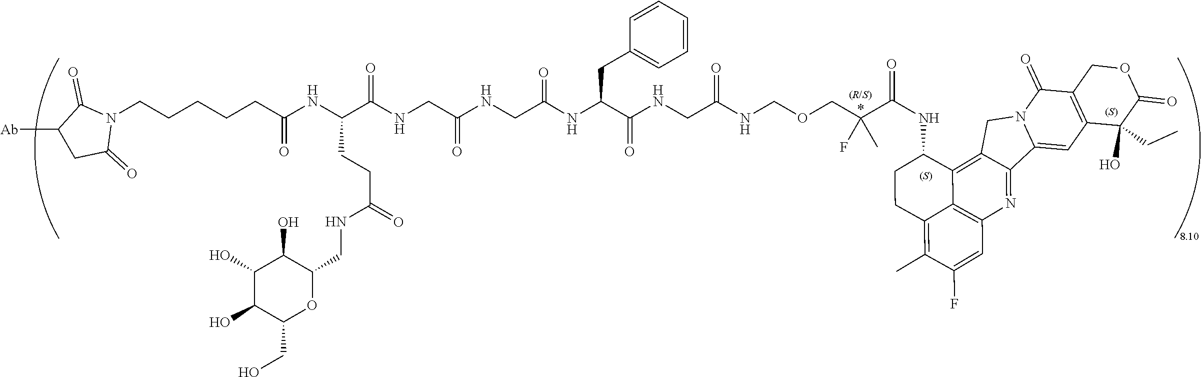 |
| ADC.1 | 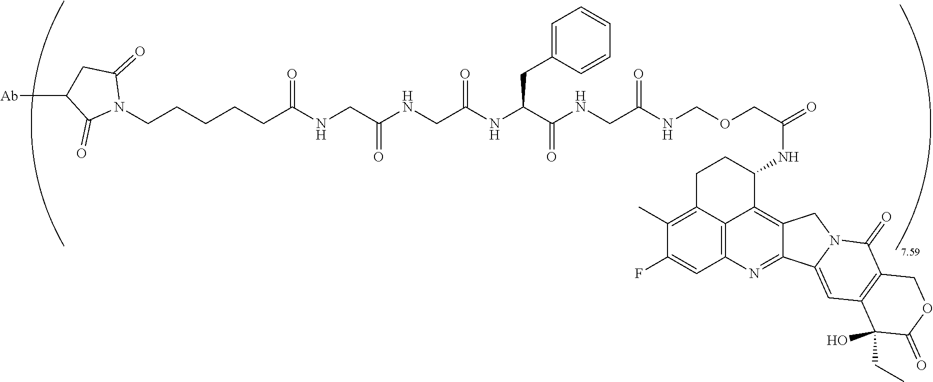 |
| ADC.2 | 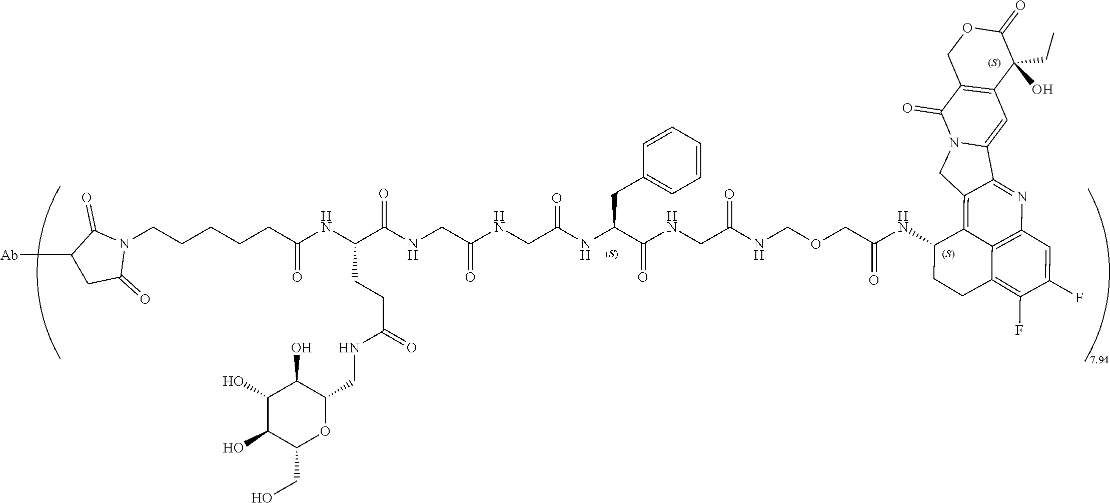 |
| ADC.3 | 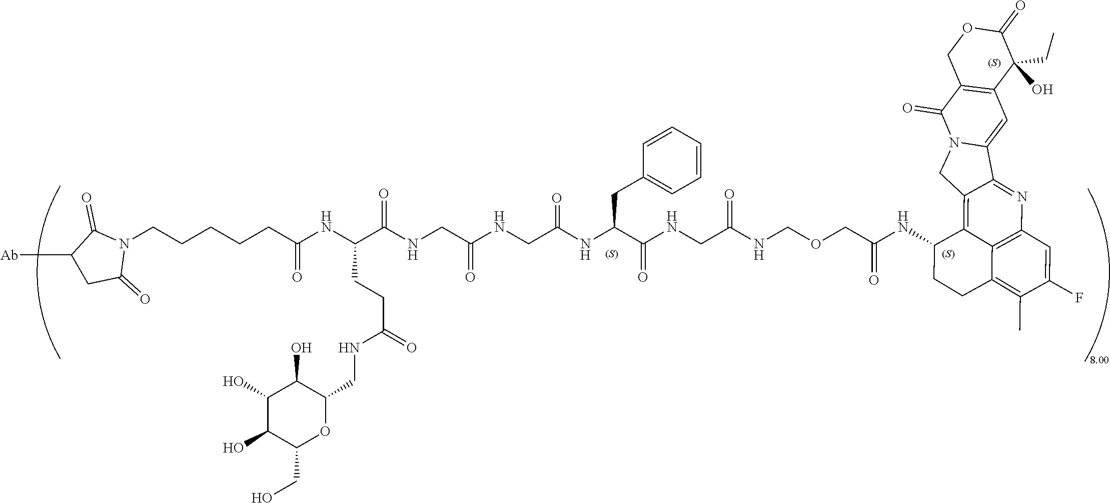 |
| ADC.4 | 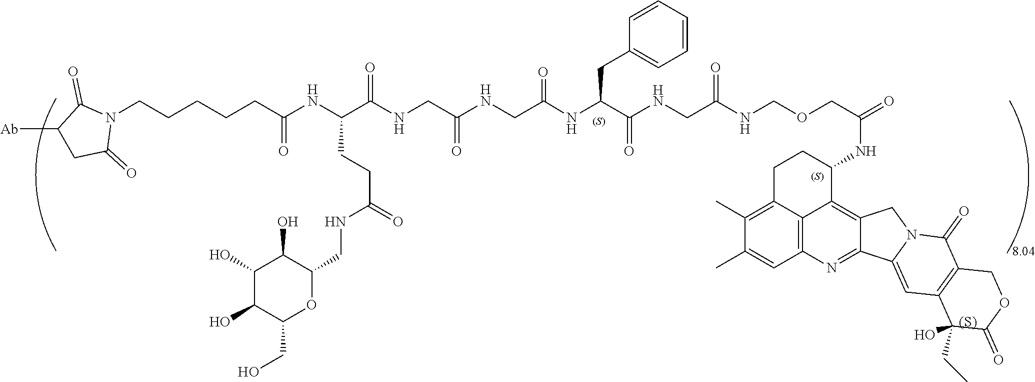 |
| ADC.5 | 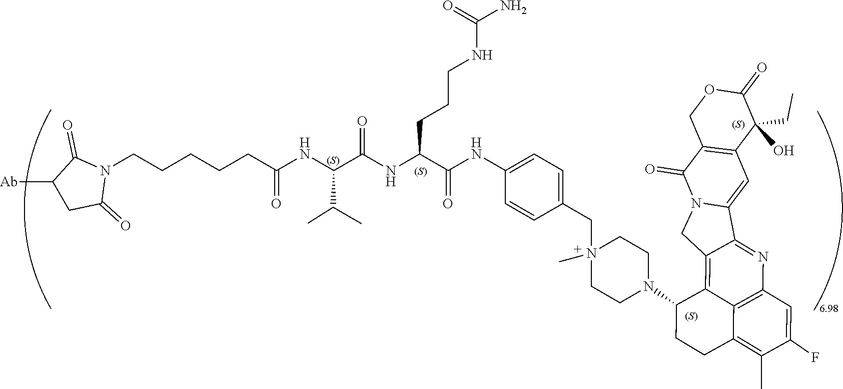 |
| ADC.6 | 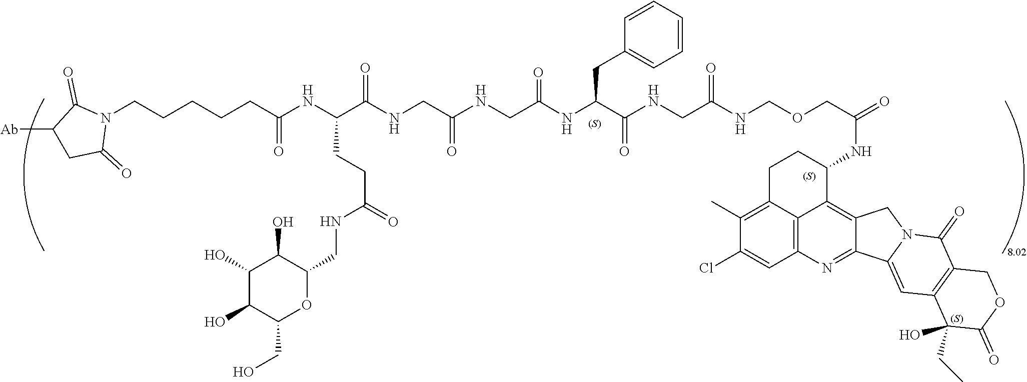 |
| ADC.7 | 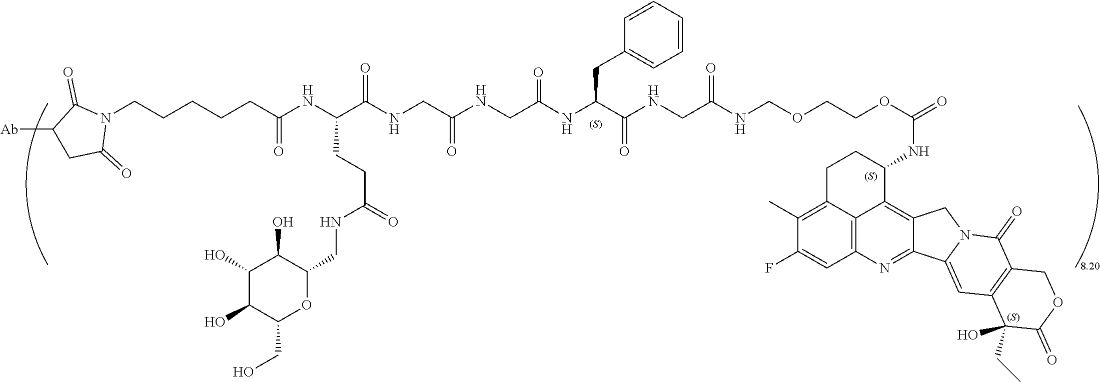 |
| ADC.8 | 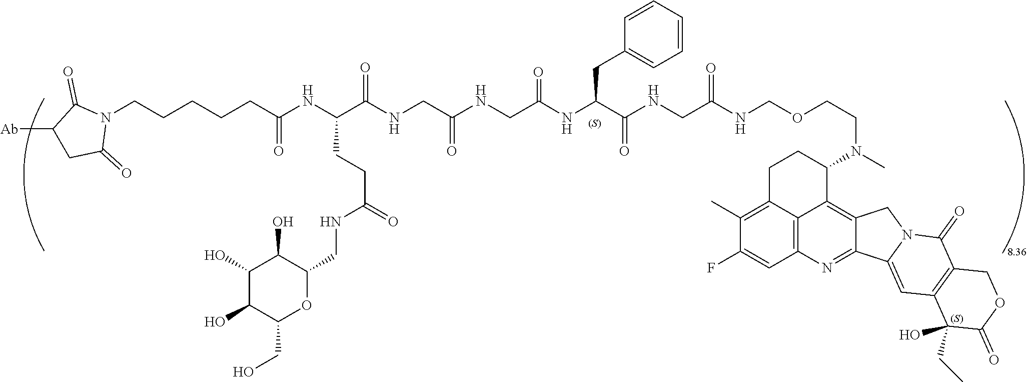 |
| ADC.9 | 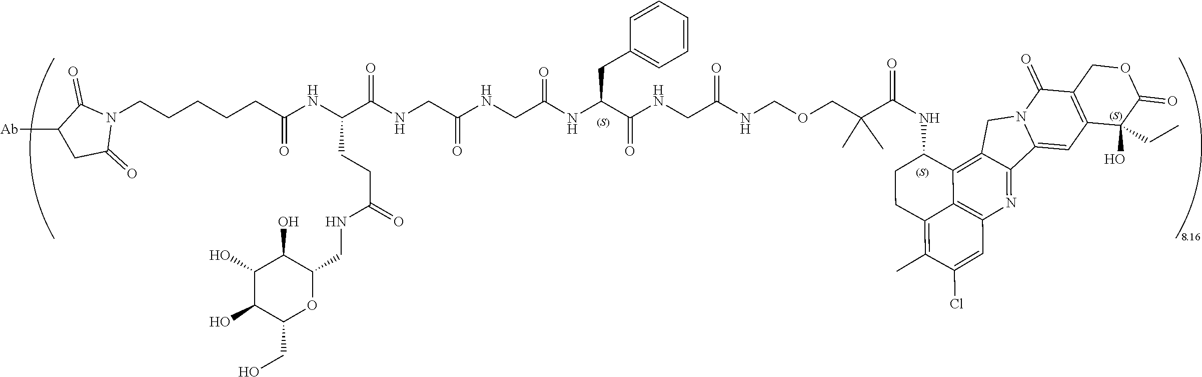 |
| ADC.10 | 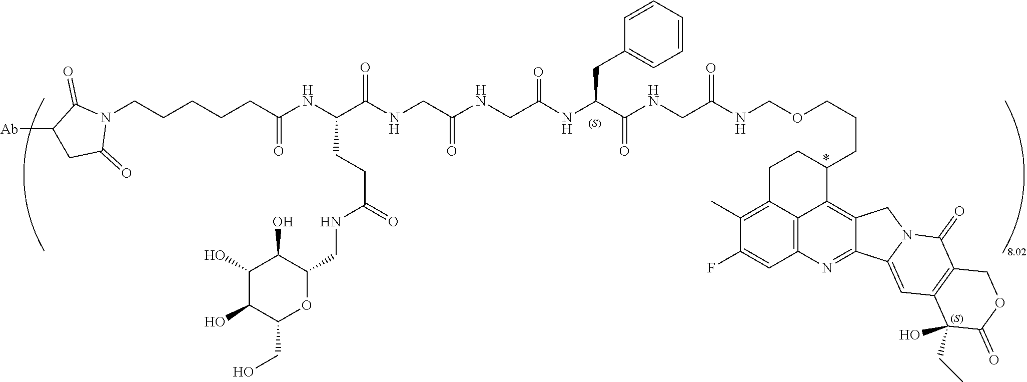 |
| ADC.11 | 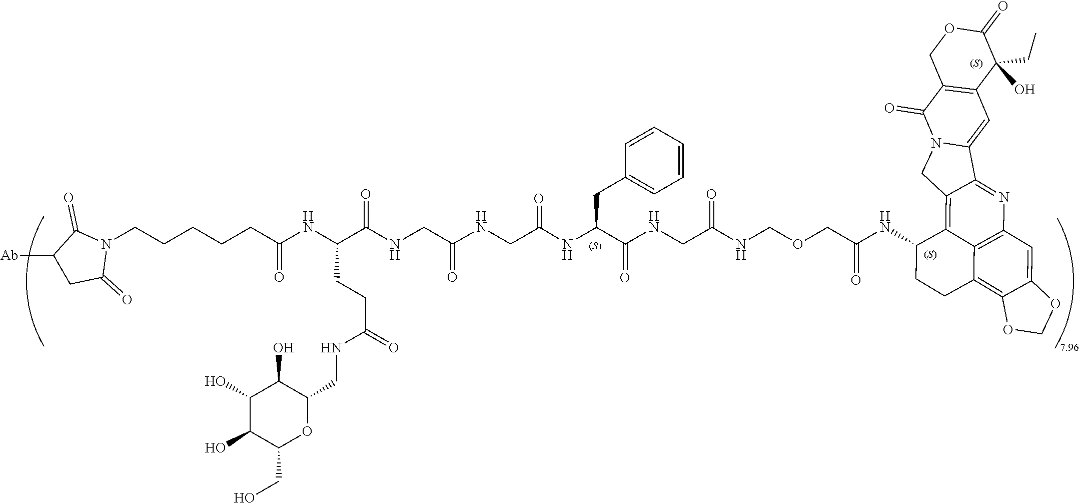 |
| ADC.12 | 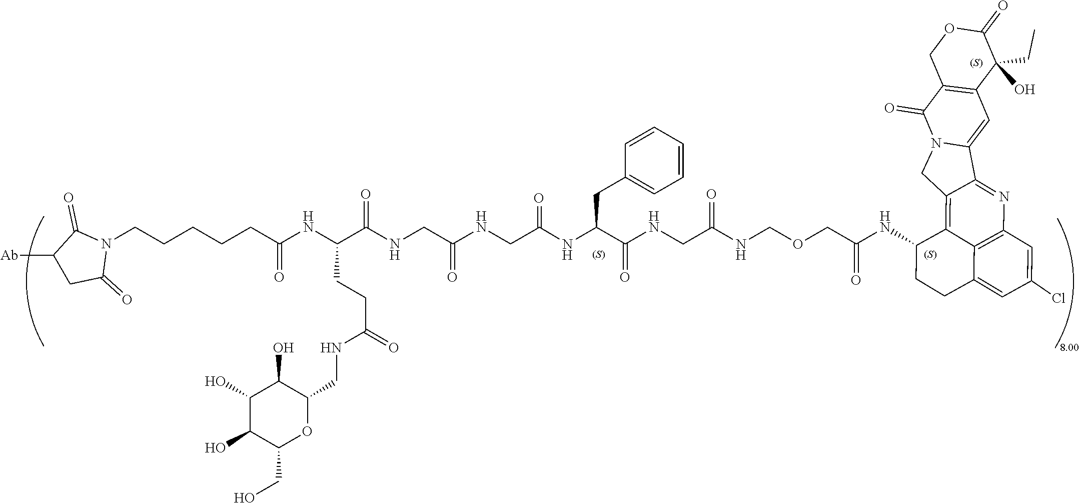 |
| ADC.13 | 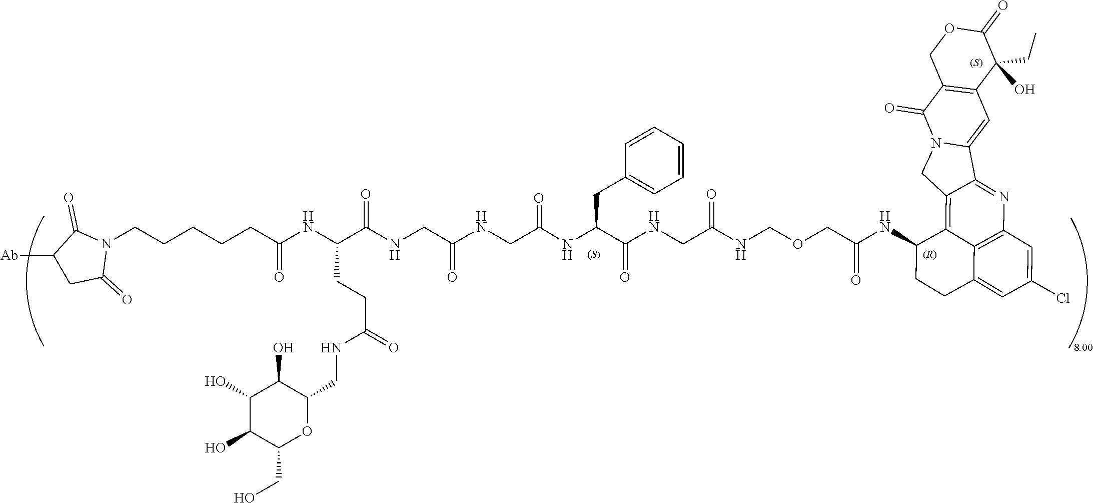 |
| ADC.14 | 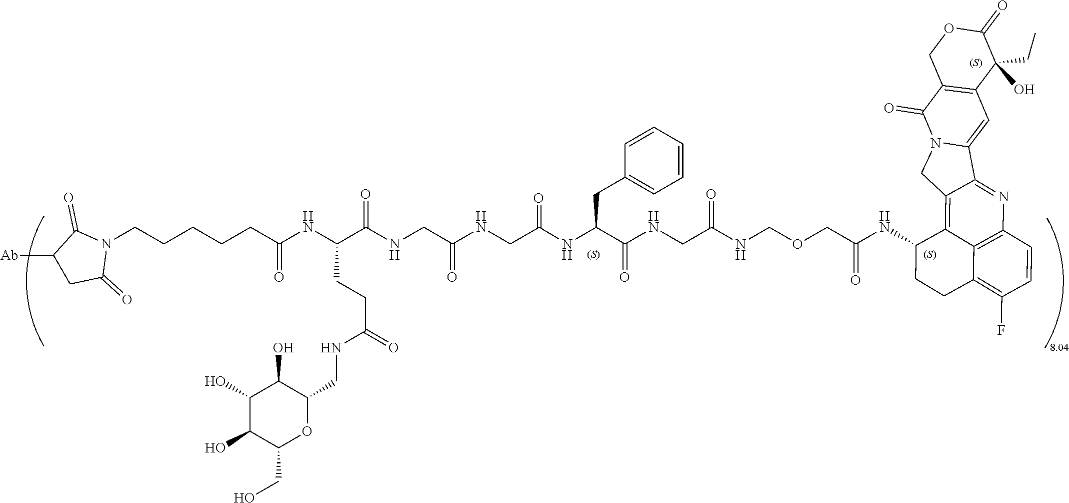 |
| ADC.15 | 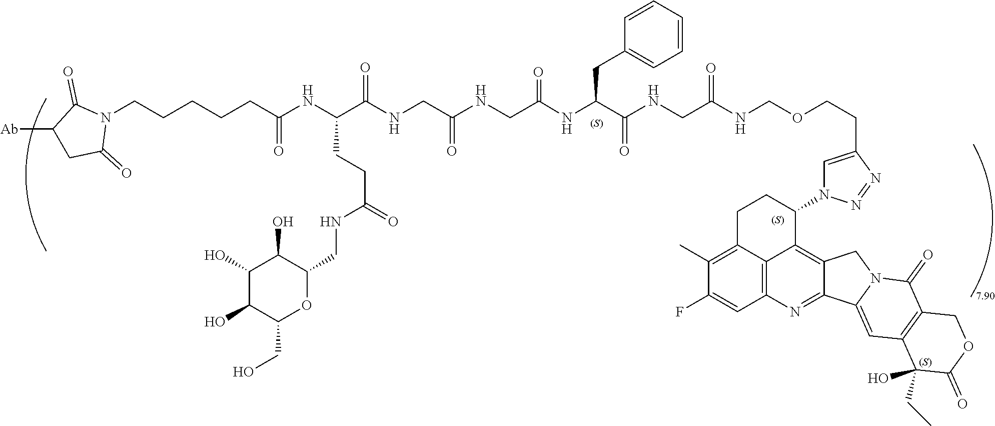 |
| ADC.16 | 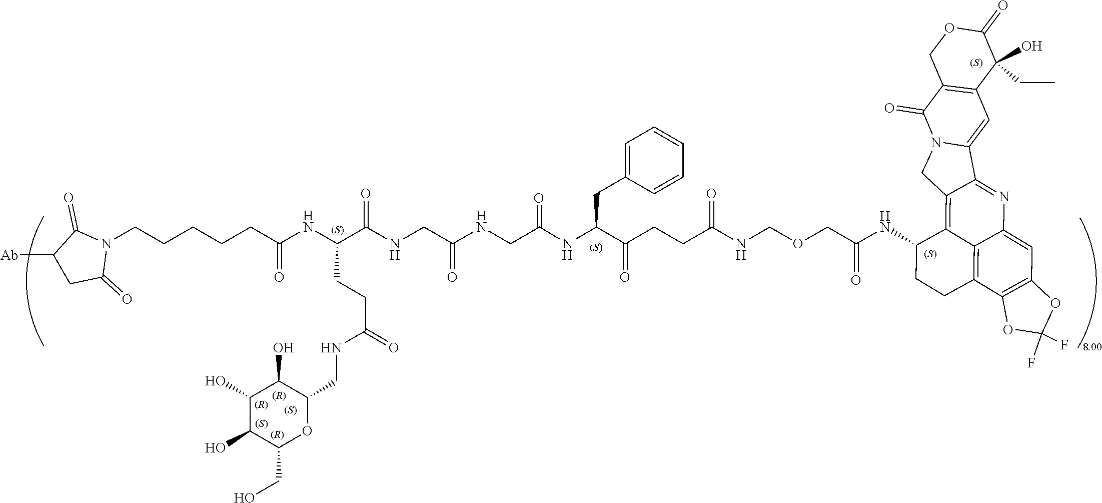 |
| ADC.17 | 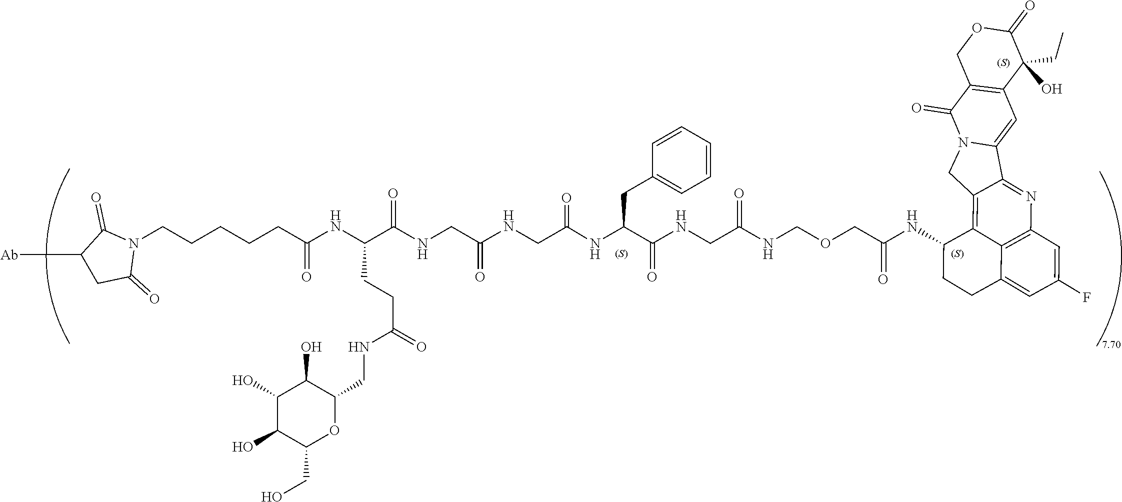 |
| ADC.18 | 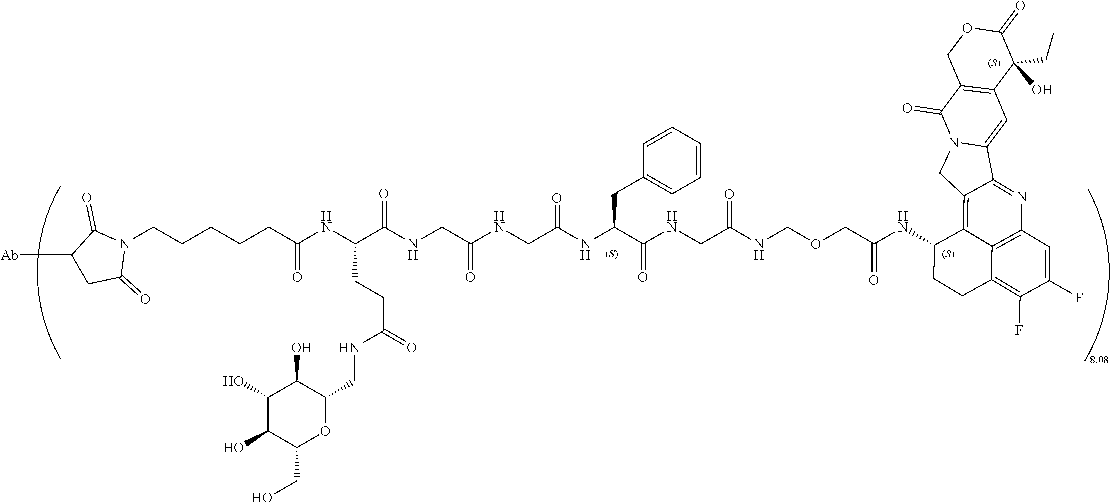 |
| ADC.19 | 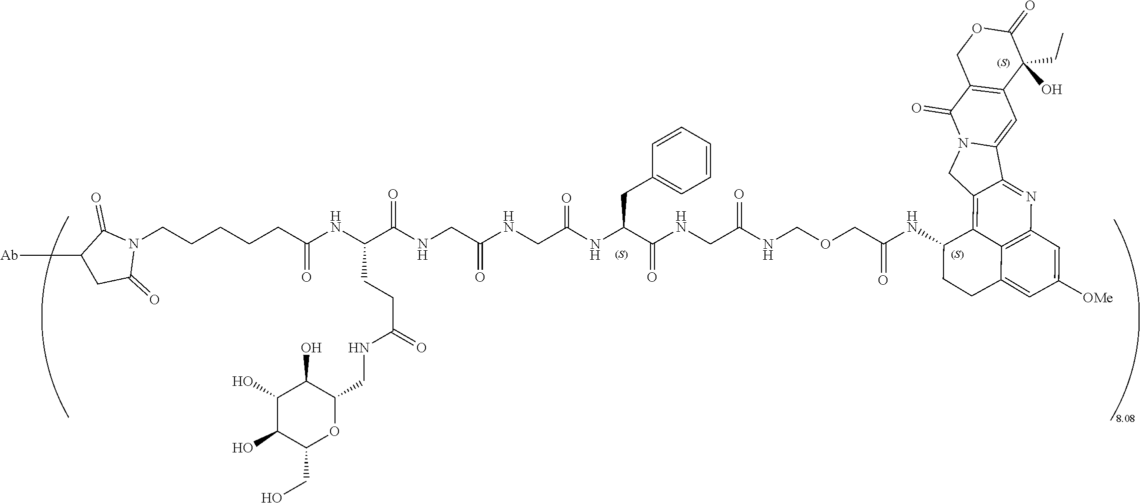 |
| ADC.20 | 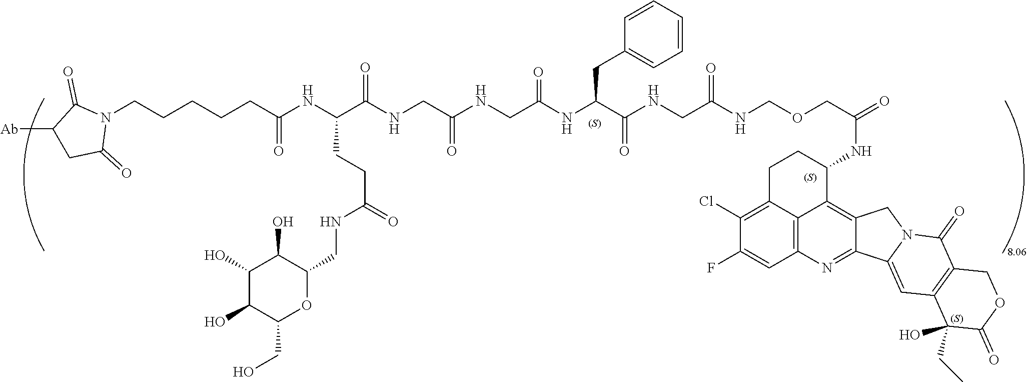 |
| ADC.21 | 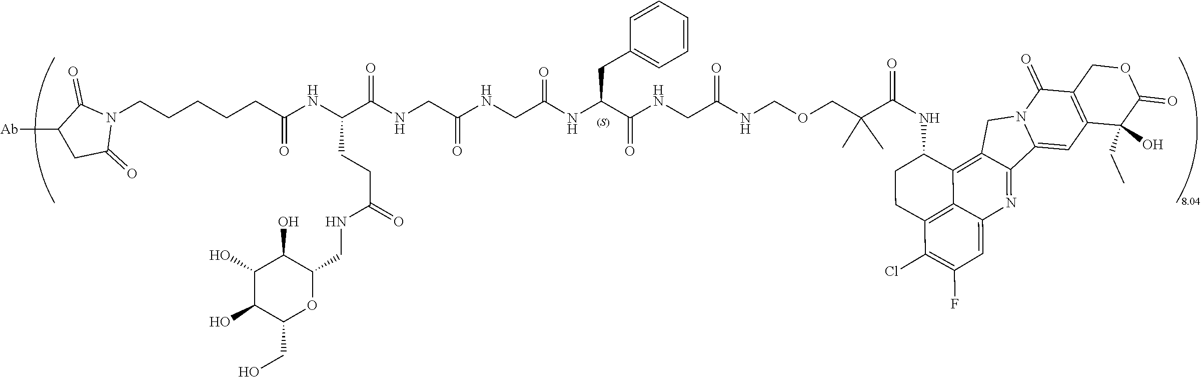 |
| ADC.22 | 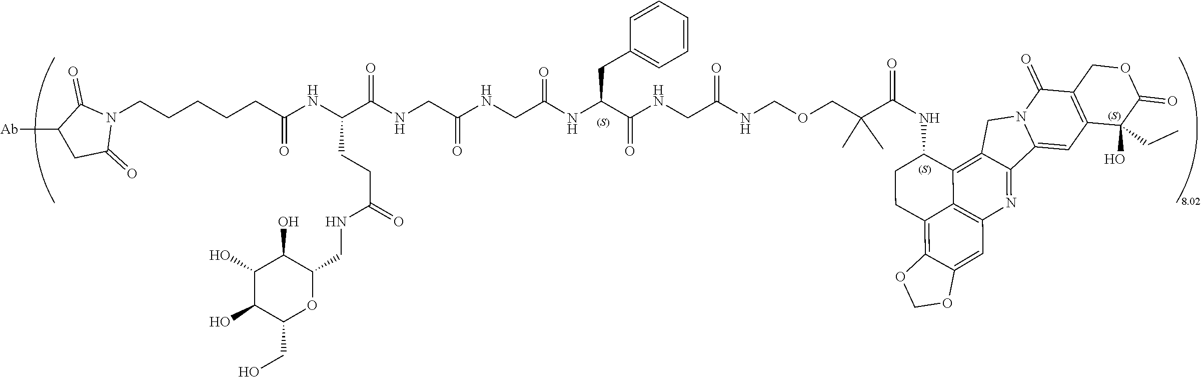 |
| ADC.23 | 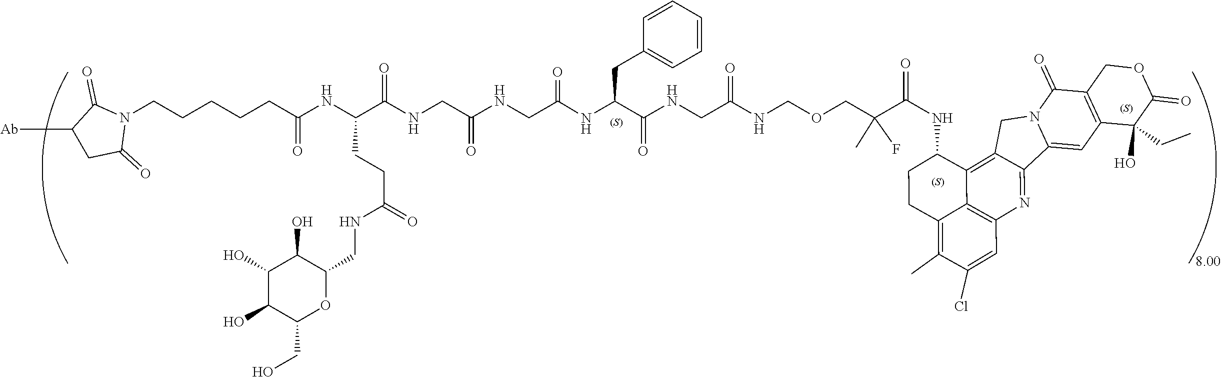 |
| ADC.24 | 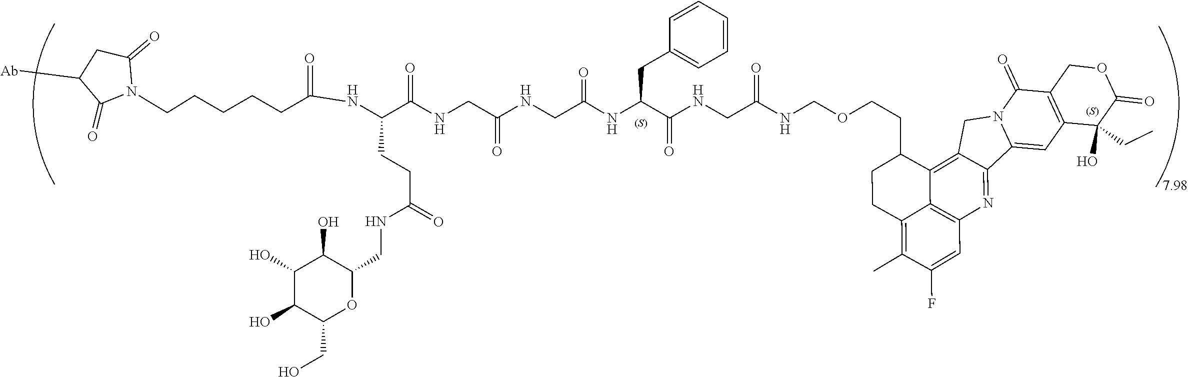 |
| ADC.25 | 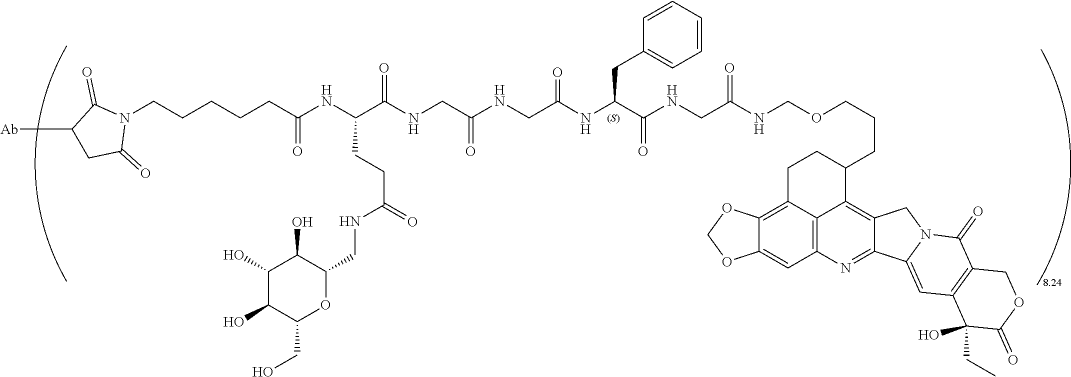 |
| ADC.26 | 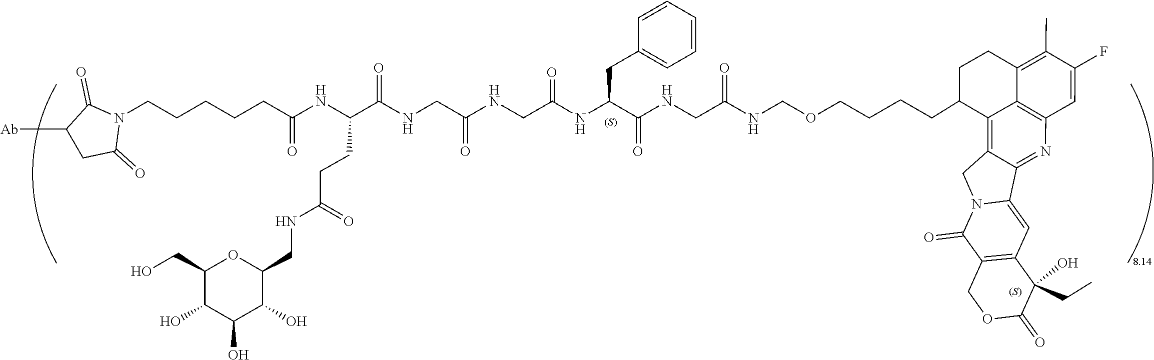 |

All possible combinations of linkers and payloads are contemplated herein. In this regard, it will be appreciated that L, used in the context of, e.g, Formula I*, V*, VIIIa*, VIIIb*, VIIIc*, encompasses C-L of a compound of Formula A or A-1. In addition, it will be appreciated that C, used in the context of Formula A or A-1, corresponds to RG¹-SP¹.

Each antibody drug conjugate may include one or more than one molecule of cytotoxic agent, such as one, two, three, four, five, six, seven, or eight molecules. The number of cytotoxic agent molecules conjugated to a single antibody or antibody fragment may be described as a drug to antibody ratio (DAR). In Formula A, Ab-(C-L-(D)ₘ)ₙ, DAR is the product of m and n.

### VIII. Methods of Making and Using Antibody Drug Conjugates

The antibody drug conjugates disclosed herein may be produced by any method known in the art. In one example, a host cell that has been transformed by an isolated nucleic acid comprising a sequence encoding an antibody or antigen-binding fragment thereof as disclosed herein is cultured under suitable culturing conditions. The antibody or antigen-binding fragment thereof is thereby expressed and may be recovered from the cell culture.

The cytotoxic agent is conjugated to the antibody or antigen-binding fragment thereof using a linker disclosed herein to produce an antibody drug conjugate. In embodiments, a conjugator is also joined to the linker, such as between the antibody and linker.

Specific methods of producing antibody drug conjugates of the present disclosure are described in the Examples.

The antibody drug conjugates of the present disclosure are useful in a variety of applications including, but not limited to, methods for the treatment of a cMET- and/or EGFR/cMET-associated disorder or disease. In one aspect, the cMET- and/or EGFR/cMET-associated disorder or disease is cancer.

In embodiments, the cancer is characterized by a cMET genetic alteration and/or constitutively active cMET signaling. The cMET genetic alteration may include cMET overexpression, genomic amplification, mutation, and/or alternative splicing. Without being limited to any mechanism or mode of action, cMET signaling may drive cancer growth. In one embodiment, the cancer is characterized by a cMET genetic alteration and the growth of the cancer is driven by cMET signaling.

In one embodiment, the cMET signaling is ligand dependent. In another embodiment, the cMET signaling is ligand independent.

In embodiments, the cancer is characterized by an EGFR activating mutation and/or the growth of the cancer cell is driven by EGFR signaling. The EGFR activating mutation may be a deletion or point mutation, including EGFR exon 19 deletion (E19del) and/or EGFR L858R/T790M.

In one embodiment, the EGFR signaling is ligand dependent. In another embodiment, the EGFR signaling is ligand independent.

In another embodiment, the conjugates including EGFR×cMET multispecific antibodies disclosed herein induce the internalization of EGFR and/or cMET receptors, thereby reducing the number of receptors on the cell surface. Thus, treatment with EGFR×cMET multispecific conjugates is not limited to diseases or disorders having a specific EGFR mutation or cMET amplification.

In certain aspects, the method comprises administering to a subject (e.g., patient) in need thereof an effective amount of an anti-cMET and/or anti-EGFR×cMET antibody drug conjugate. The subject can include, without limitation, a subject with any of the foregoing cancers. In embodiments, the cancer is a gastric cancer, a colorectal cancer, or a lung cancer. The lung cancer may be non-small cell lung cancer (NSCLC) or small cell lung cancer (SCLC). The NSCLC may be squamous non-small cell lung cancer. In another embodiment, the gastric cancer may be alpha-fetoprotein positive (AFP+) gastric cancer.

The antibody drug conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments, or antibody drug conjugates, of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### VIII. Pharmaceutical Compositions and Formulations

Also provided are compositions, including pharmaceutical formulations, comprising an drug conjugate comprising an antibody or antigen-binding fragment thereof as disclosed herein, or polynucleotides comprising sequences encoding an antibody or antigen-binding fragment as disclosed herein, and a toxic drug conjugate. These compositions can further comprise suitable carriers, such as pharmaceutically acceptable excipients including buffers, which are well known in the art.

Pharmaceutical formulations of antibody drug conjugates as described herein are prepared by mixing such antibody or antigen-binding fragment and antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody or antibody drug conjugate, which matrices are in the form of shaped articles, e.g. films, or microcapsules.

The formulations to be used for in vivo administration are generally sterile. Sterility can be readily accomplished, e.g., by filtration through sterile filtration membranes.

### EXAMPLES

TABLE 3
List of Abbreviations

| | |
|---|---|
| DCM | Dichloromethane |
| DIPEA/ | N,N-diisopropylethylamine |
| DIEA | |
| DMF | N,N-dimethylformamide |
| EtOAc | Ethyl acetate |
| FA | Formic acid |
| HATU | O-(7-Azabenzotriazol-1-yl)-N,N,N′,N′-tetramethyluronium |
| | hexafluorophosphate |
| HOAc | Acetic acid |
| LCMS | Liquid chromatography mass spectrometry |
| MeCN/ACN | Acetonitrile |
| MeOH | Methanol |
| MTBE | Methyl tert-butyl ether |
| Pd/C | Palladium on carbon |
| Prep-HPLC | Preparative high performance liquid chromatography |
| r.t. | Room temperature |
| TFA | Trifluoroacetic acid |
| THF | Tetrahydrofuran |
| TLC | Thin-layer chromatography |
| UPLC | Ultra-performance liquid chromatography |

### Example 1. Generation of Anti-cMET Antibodies

### Mouse Immunization

To generate anti-cMET antibodies, genetically engineered mice (RenLite®, Biocytogen) were immunized by human cMET extracellular domain (amino acids 1-932 of SEQ ID NO: 69 which is full length human cMET) fused to his tag or mouse Ec (internal generated, SEQ ID NOS: 1 and 2) mixed with adjuvants (Complete Ereund's Adjuvant (E5881, Sigma) for prime immune and Ereund's Adjuvant, Incomplete (E5506, Sigma) for the following immune). Animals were injected bi- or tri-weekly intraperitoneally and subcutaneously. The mice used comprise DNA encoding different human immunoglobulin heavy chain and one shared human light chain variable region.

Serum titers against cMET protein were determined by ELISA to monitor the humoral immune response. Animals with sufficient cMET specific antibody titers were administered a final boost of cMET protein for the antibody screen.

TABLE 4
Antigen Sequence List

| Antigen | SEQ ID NO | | SEQUENCE |
|---|---|---|---|
| Human | SEQ ID NO: 1 | AA | ECKEALAKSEMNVNMKYQLPNFTAETPIQNVILHEH |
| cMET-ECD- | | sequence | HIFLGATNYIYVLNEEDLQKVAEYKTGPVLEHPDCFP |
| his | | | CQDCSSKANLSGGVWKDNINMALVVDTYYDDQLIS |
| | | | CGSVNRGTCQRHVFPHNHTADIQSEVHCIFSPQIEEPS |
| | | | QCPDCVVSALGAKVLSSVKDRFINFFVGNTINSSYFP |
| | | | DHPLHSISVRRLKETKDGFMFLTDQSYIDVLPEFRDS |
| | | | YPIKYVHAFESNNFIYFLTVQRETLDAQTFHTRIIRFC |
| | | | SINSGLHSYMEMPLECILTEKRKKRSTKKEVFNILQA |
| | | | AYVSKPGAQLARQIGASLNDDILFGVFAQSKPDSAEP |
| | | | MDRSAMCAFPIKYVNDFFNKIVNKNNVRCLQHFYG |
| | | | PNHEHCFNRTLLRNSSGCEARRDEYRTEFTTALQRVD |
| | | | LFMGQFSEVLLTSISTFIKGDLTIANLGTSEGRFMQVV |
| | | | VSRSGPSTPHVNFLLDSHPVSPEVIVEHTLNQNGYTL |
| | | | VITGKKITKIPLNGLGCRHFQSCSQCLSAPPFVQCGW |
| | | | CHDKCVRSEECLSGTWTQQICLPAIYKVFPNSAPLEG |
| | | | GTRLTICGWDFGFRRNNKFDLKKTRVLLGNESCTLTL |
| | | | SESTMNTLKCTVGPAMNKHFNMSIIISNGHGTTQYST |
| | | | FSYVDPVITSISPKYGPMAGGTLLTLTGNYLNSGNSR |
| | | | HISIGGKTCTLKSVSNSILECYTPAQTISTEFAVKLKID |
| | | | LANRETSIFSYREDPIVYEIHPTKSFISGGSTITGVGKN |
| | | | LNSVSVPRMVINVHEAGRNFTVACQHRSNSEIICCTT |
| | | | PSLQQLNLQLPLKTKAFFMLDGILSKYFDLIYVHNPV |
| | | | FKPFEKPVMISMGNENVLEIKGNDIDPEAVKGEVLKV |
| | | | GNKSCENIHLHSEAVLCTVPNDLLKLNSELNIEWKQ |
| | | | AISSTVLGKVIVQPDQNFTHHHHHH |
| Human | SEQ ID NO: 2 | AA | ECKEALAKSEMNVNMKYQLPNFTAETPIQNVILHEH |
| cMET-ECD- | | sequence | HIFLGATNYIYVLNEEDLQKVAEYKTGPVLEHPDCFP |
| mFc | | | CQDCSSKANLSGGVWKDNINMALVVDTYYDDQLIS |
| | | | CGSVNRGTCQRHVFPHNHTADIQSEVHCIFSPQIEEPS |
| | | | QCPDCVVSALGAKVLSSVKDRFINFFVGNTINSSYFP |
| | | | DHPLHSISVRRLKETKDGFMFLTDQSYIDVLPEFRDS |
| | | | YPIKYVHAFESNNFIYFLTVQRETLDAQTFHTRIIRFC |
| | | | SINSGLHSYMEMPLECILTEKRKKRSTKKEVFNILQA |
| | | | AYVSKPGAQLARQIGASLNDDILFGVFAQSKPDSAEP |
| | | | MDRSAMCAFPIKYVNDFFNKIVNKNNVRCLQHFYG |
| | | | PNHEHCFNRTLLRNSSGCEARRDEYRTEFTTALQRVD |
| | | | LFMGQFSEVLLTSISTFIKGDLTIANLGTSEGRFMQVV |
| | | | VSRSGPSTPHVNFLLDSHPVSPEVIVEHTLNQNGYTL |
| | | | VITGKKITKIPLNGLGCRHFQSCSQCLSAPPFVQCGW |
| | | | CHDKCVRSEECLSGTWTQQICLPAIYKVFPNSAPLEG |
| | | | GTRLTICGWDFGFRRNNKFDLKKTRVLLGNESCTLTL |
| | | | SESTMNTLKCTVGPAMNKHFNMSIIISNGHGTTQYST |
| | | | FSYVDPVITSISPKYGPMAGGTLLTLTGNYLNSGNSR |
| | | | HISIGGKTCTLKSVSNSILECYTPAQTISTEFAVKLKID |
| | | | LANRETSIFSYREDPIVYEIHPTKSFISGGSTITGVGKN |
| | | | LNSVSVPRMVINVHEAGRNFTVACQHRSNSEIICCTT |
| | | | PSLQQLNLQLPLKTKAFFMLDGILSKYFDLIYVHNPV |
| | | | FKPFEKPVMISMGNENVLEIKGNDIDPEAVKGEVLKV |
| | | | GNKSCENIHLHSEAVLCTVPNDLLKLNSELNIEWKQ |
| | | | AISSTVLGKVIVQPDQNFTGGGGSCPPCKCPAPNLLG |
| | | | GPSVFIFPPKIKDVLMISLSPIVTCVVVDVSEDDPDVQ |
| | | | ISWFVNNVEVHTAQTQTHREDYNSTLRVVSALPIQH |
| | | | QDWMSGKEFKCKVNNKDLPAPIERTISKPKGSVRAP |
| | | | QVYVLPPPEEEMTKKQVTLTCMVTDFMPEDIYVEW |
| | | | TNNGKTELNYKNTEPVLDSDGSYFMYSKLRVEKKN |
| | | | WVERNSYSCSVVHEGLHNHHTTKSFSRTPGK |
| cyno cMET- | SEQ ID NO: 3 | AA | ECKEALAKSEMNVNMKYQLPNFTAETAIQNVILHEH |
| ECD-his | | sequence | HIFLGATNYIYVLNEEDLQKVAEYKTGPVLEHPDCFP |
| | | | CQDCSSKANLSGGVWKDNINMALVVDTYYDDQLIS |
| | | | CGSVNRGTCQRHVFPHNHTADIQSEVHCIFSPQIEEP |
| | | | NQCPDCVVSALGAKVLSSVKDRFINFFVGNTINSSYF |
| | | | PHHPLHSISVRRLKETKDGFMFLTDQSYIDVLPEFRD |
| | | | SYPIKYIHAFESNNFIYFLTVQRETLNAQTFHTRIIRFC |
| | | | SLNSGLHSYMEMPLECILTEKRKKRSTKKEVFNILQA |
| | | | AYVSKPGAQLARQIGASLNDDILFGVFAQSKPDSAEP |
| | | | MDRSAMCAFPIKYVNDFFNKIVNKNNVRCLQHFYG |
| | | | PNHEHCFNRTLLRNSSGCEARRDEYRAEFTTALQRV |
| | | | DLFMGQFSEVLLTSISTFVKGDLTIANLGTSEGRFMQ |
| | | | VVVSRSGPSTPHVNFLLDSHPVSPEVIVEHPLNQNGY |
| | | | TLVVTGKKITKIPLNGLGCRHFQSCSQCLSAPPFVQC |
| | | | GWCHDKCVRSEECPSGTWTQQICLPAIYKVFPTSAPL |
| | | | EGGTRLTICGWDFGFRRNNKFDLKKTRVLLGNESCT |
| | | | LTLSESTMNTLKCTVGPAMNKHFNMSIIISNGHGTTQ |
| | | | YSTFSYVDPIITSISPKYGPMAGGTLLTLTGNYLNSGN |
| | | | SRHISIGGKTCTLKSVSNSILECYTPAQTISTEFAVKLK |
| | | | IDLANRETSIFSYREDPIVYEIHPTKSFISGGSTITGVG |
| | | | KNLHSVSVPRMVINVHEAGRNFTVACQHRSNSEIICC |
| | | | TTPSLQQLNLQLPLKTKAFFMLDGILSKYFDLIYVHN |
| | | | PVFKPFEKPVMISMGNENVLEIKGNDIDPEAVKGEVL |
| | | | KVGNKSCENIHLHSEAVLCTVPNDLLKLNSELNIEW |
| | | | KQAISSTVLGKVIVQPDQNFTHHHHHH |
| Human | SEQ ID NO: 69 | AA | MKAPAVLAPGILVLLFTLVQRSNGECKEALAKSEMN |
| cMET full | | sequence | VNMKYQLPNFTAETPIQNVILHEHHIFLGATNYIYVL |
| length | | | NEEDLQKVAEYKTGPVLEHPDCFPCQDCSSKANLSG |
| | | | GVWKDNINMALVVDTYYDDQLISCGSVNRGTCQRH |
| | | | VFPHNHTADIQSEVHCIFSPQIEEPSQCPDCVVSALGA |
| | | | KVLSSVKDRFINFFVGNTINSSYFPDHPLHSISVRRLK |
| | | | ETKDGFMFLTDQSYIDVLPEFRDSYPIKYVHAFESNN |
| | | | FIYFLTVQRETLDAQTFHTRIIRFCSINSGLHSYMEMP |
| | | | LECILTEKRKKRSTKKEVFNILQAAYVSKPGAQLARQ |
| | | | IGASLNDDILFGVFAQSKPDSAEPMDRSAMCAFPIKY |
| | | | VNDFFNKIVNKNNVRCLQHFYGPNHEHCFNRTLLRN |
| | | | SSGCEARRDEYRTEFTTALQRVDLFMGQFSEVLLTSIS |
| | | | TFIKGDLTIANLGTSEGRFMQVVVSRSGPSTPHVNFL |
| | | | LDSHPVSPEVIVEHTLNQNGYTLVITGKKITKIPLNGL |
| | | | GCRHFQSCSQCLSAPPFVQCGWCHDKCVRSEECLSG |
| | | | TWTQQICLPAIYKVFPNSAPLEGGTRLTICGWDFGFR |
| | | | RNNKFDLKKTRVLLGNESCTLTLSESTMNTLKCTVG |
| | | | PAMNKHFNMSIIISNGHGTTQYSTFSYVDPVITSISPK |
| | | | YGPMAGGTLLTLTGNYLNSGNSRHISIGGKTCTLKSV |
| | | | SNSILECYTPAQTISTEFAVKLKIDLANRETSIFSYRED |
| | | | PIVYEIHPTKSFISGGSTITGVGKNLNSVSVPRMVINV |
| | | | HEAGRNFTVACQHRSNSEIICCTTPSLQQLNLQLPLK |
| | | | TKAFFMLDGILSKYFDLIYVHNPVFKPFEKPVMISMG |
| | | | NENVLEIKGNDIDPEAVKGEVLKVGNKSCENIHLHSE |
| | | | AVLCTVPNDLLKLNSELNIEWKQAISSTVLGKVIVQP |
| | | | DQNFTGLIAGVVSISTALLLLLGFFLWLKKRKQIKDL |
| | | | GSELVRYDARVHTPHLDRLVSARSVSPTTEMVSNES |
| | | | VDYRATFPEDQFPNSSQNGSCRQVQYPLTDMSPILTS |
| | | | GDSDISSPLLQNTVHIDLSALNPELVQAVQHVVIGPSS |
| | | | LIVHFNEVIGRGHFGCVYHGTLLDNDGKKIHCAVKS |
| | | | LNRITDIGEVSQFLTEGIIMKDFSHPNVLSLLGICLRSE |
| | | | GSPLVVLPYMKHGDLRNFIRNETHNPTVKDLIGFGL |
| | | | QVAKGMKYLASKKFVHRDLAARNCMLDEKFTVKV |
| | | | ADFGLARDMYDKEYYSVHNKTGAKLPVKWMALES |
| | | | LQTQKFTTKSDVWSFGVLLWELMTRGAPPYPDVNT |
| | | | FDITVYLLQGRRLLQPEYCPDPLYEVMLKCWHPKAE |
| | | | MRPSFSELVSRISAIFSTFIGEHYVHVNATYVNVKCVA |
| | | | PYPSLLSSEDNADDEVDTRPASFWETS |

### Plasma Cell Screening by the Beacon Optofluidic System

At 3-5 days post final boost, spleens were collected and mashed into single cell suspensions. Plasma cells were isolated with a mouse CD138 positive selection kit (STEMCELL™) according to the manufacturer's instructions. Enriched plasma cells with a density of 6.25×10⁶/ml were imported into the channel and penned into the NanoPen chambers of OptoSelect 14K Chip™ (Berkeley Lights) according to the manufacturer's instructions. To screen human cMET specific plasma cells, human cMET protein (MET-H82E1, Acrobiosystems) conjugated beads (520-00053, Berkeley Lights) and Alexa Fluor 488 goat anti-mouse IgG secondary antibody (Jackson ImmunoResearch) with a concentration of 5 μg/ml were imported into the channel. Following import, the freeze valve was turned on and the exposure time of FITC channel for Alexa Fluor 488 fluorophore was set to 1000 ms. Bloom-like positive signals were captured by time lapse imaging with settings of 3-minute period and 10 cycles. After completion of human cMET specific plasma cell screening, cyno cMET specific plasma cells were screened with cyno cMET-ECD-his (in house generated, SEQ ID NO: 3) conjugated beads. An assay to remove non-specific binding signals using irrelevant His-tagged protein-conjugated beads was performed as a counter screen. Plasma cells showing both human and cyno cMET specific positive signals were individually exported into 96-well plates filled with lysis buffer.

### Example 2. Antibody VH and VL Gene Cloning, Sequencing, and Expression

First-strand cDNA was synthesized, and total cDNA was amplified with Opto Plasma B Discovery cDNA Synthesis Kit™ (Berkeley Lights) according to the manufacturer's instructions. Antibody VH and VL genes were amplified with Opto Plasma B Discovery Sanger Prep Kit™ (Berkeley Lights) according to the manufacturer's instructions. The amplified VH and VL genes were cloned into mammalian expression vector containing human IgG1 constant region (SEQ ID NO: 70) and human kappa chain constant region (SEQ ID NO: 71) genes and sequenced, respectively. The amino acid sequences of the 3 HCDRs, 3 LCDRs, VH and VL and the DNA sequences of the VH and VL for representative antibodies are listed in Table 5 and Table 6 as SEQ ID Nos: 4 to 68. Antibodies were expressed by Expi293™ cells and purified by affinity chromatography.

TABLE 5
Anti-cMET Monoclonal Antibody List

| | SEQ ID NO. | | | | | | | | | |
|---|---|---|---|---|---|---|---|---|---|---|
| | VH | VH | HCDR1 | HCDR2 | HCDR3 | VL | VL | LCDR1 | LCDR2 | LCDR3 |
| Antibody | (AA) | (DNA) | (Kabat) | (Kabat) | (Kabat) | (AA) | (DNA) | (Kabat) | (Kabat) | (Kabat) |
| 063Ab10910 | 4 | 5 | 6 | 7 | 8 | 64 | 65 | 66 | 67 | 68 |
| 061Ab15310 | 9 | 10 | 11 | 12 | 13 | 64 | 65 | 66 | 67 | 68 |
| 063Ab16010 | 14 | 15 | 16 | 17 | 18 | 64 | 65 | 66 | 67 | 68 |
| 063Ab02110 | 19 | 20 | 21 | 22 | 23 | 64 | 65 | 66 | 67 | 68 |
| 063Ab15210 | 24 | 25 | 26 | 27 | 28 | 64 | 65 | 66 | 67 | 68 |
| 062Ab16310 | 29 | 30 | 31 | 32 | 33 | 64 | 65 | 66 | 67 | 68 |
| 063Ab05510 | 34 | 35 | 36 | 37 | 38 | 64 | 65 | 66 | 67 | 68 |
| 063Ab07710 | 39 | 40 | 41 | 42 | 43 | 64 | 65 | 66 | 67 | 68 |
| 063Ab14710 | 44 | 45 | 46 | 47 | 48 | 64 | 65 | 66 | 67 | 68 |
| 061Ab05110 | 49 | 50 | 51 | 52 | 53 | 64 | 65 | 66 | 67 | 68 |
| 063Ab03210 | 54 | 55 | 56 | 57 | 58 | 64 | 65 | 66 | 67 | 68 |
| 063Ab16720 | 59 | 60 | 61 | 62 | 63 | 64 | 65 | 66 | 67 | 68 |

TABLE 6
Anti-cMET Monoclonal Antibody Sequence List

| | SEQ ID | | |
|---|---|---|---|
| Antibody | NO | Description | SEQUENCE |
| 063Ab10910 | SEQ ID | VH (AA) | QVQLVESGGGVVQPGRSLRLSCAASGFTFSSHGMHWVRQAP |
| | NO: 4 | | GKGLEWVAVISYDESDKYYADSVKGRFTISRDHSKNTLYLQM |
| | | | NSLRAEDTAVYYCVKDRNMGYSGWLDYWGQGTLVTVSS |
| | SEQ ID | VH (DNA) | CAGGTGCAGCTGGTGGAGTCTGGGGGAGGCGTGGTCCAGC |
| | NO: 5 | | CTGGGAGGTCCCTGAGACTCTCCTGTGCAGCCTCTGGATTC |
| | | | ACCTTCAGTAGCCATGGCATGCACTGGGTCCGCCAGGCTCC |
| | | | AGGCAAGGGGCTGGAGTGGGTGGCAGTTATATCATATGAT |
| | | | GAAAGTGATAAATATTATGCAGACTCCGTGAAGGGCCGAT |
| | | | TCACCATCTCCAGAGACCATTCCAAGAACACGCTGTATCTG |
| | | | CAAATGAACAGCCTGAGAGCTGAGGACACGGCTGTATATT |
| | | | ACTGTGTGAAAGATCGGAATATGGGATATAGTGGCTGGCTT |
| | | | GACTACTGGGGCCAGGGAACCCTGGTCACCGTCTCCTCA |
| | SEQ ID | HCDR1 | SHGMH |
| | NO: 6 | (Kabat) | |
| | SEQ ID | HCDR2 | VISYDESDKYYADSVKG |
| | NO: 7 | (Kabat) | |
| | SEQ ID | HCDR3 | DRNMGYSGWLDY |
| | NO: 8 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 061Ab15310 | SEQ ID | VH (AA) | QVQLVESGGGVVQPGKSLRLSCAASGFTFSSSGLHWVRQAPG |
| | NO: 9 | | KGLEWVAVISYDTSNKYYADSVKGRFTISRDNSKNTLYLQMN |
| | | | SLRAEDTAVYYCAKDLNRGYDWGFDYWGQGTLVTVSS |
| | SEQ ID | VH (DNA) | CAGGTGCAGCTGGTGGAGTCTGGGGGAGGCGTGGTCCAGC |
| | NO: 10 | | CTGGGAAGTCCCTGAGACTCTCCTGTGCAGCCTCTGGATTC |
| | | | ACCTTCAGTAGTTCTGGCTTGCACTGGGTCCGCCAGGCTCC |
| | | | AGGCAAGGGACTGGAGTGGGTGGCAGTTATATCATATGAT |
| | | | ACAAGTAATAAATACTATGCAGACTCCGTGAAGGGCCGAT |
| | | | TCACCATCTCCAGAGACAATTCCAAGAACACGCTGTATCTG |
| | | | CAAATGAACAGCCTGAGAGCTGAGGACACGGCTGTATATT |
| | | | ACTGTGCGAAAGATCTAAATCGTGGCTATGATTGGGGGTTT |
| | | | GACTACTGGGGCCAGGGAACCCTGGTCACCGTCTCCTCA |
| | SEQ ID | HCDR1 | SSGLH |
| | NO: 11 | (Kabat) | |
| | SEQ ID | HCDR2 | VISYDTSNKYYADSVKG |
| | NO: 12 | (Kabat) | |
| | SEQ ID | HCDR3 | DLNRGYDWGFDY |
| | NO: 13 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab16010 | SEQ ID | VH (AA) | QVQLVESGGGVVQPGRSLRLSCAASGFTFSNYGMHWVRQAP |
| | NO: 14 | | GKGLEWVAVISYDGVDKYYADSVKGRFTISRDNSKNTLYLQ |
| | | | MNSLRAEDTAMYYCAKDRISYNWNLDYWGQGTLVTVSS |
| | SEQ ID | VH (DNA) | CAGGTGCAGCTGGTGGAGTCTGGGGGAGGCGTGGTCCAGC |
| | NO: 15 | | CTGGGAGGTCCCTGAGACTCTCCTGTGCAGCCTCTGGATTC |
| | | | ACCTTCAGTAACTATGGCATGCACTGGGTCCGCCAGGCTCC |
| | | | AGGCAAGGGGCTGGAGTGGGTGGCAGTTATATCATATGAT |
| | | | GGCGTTGATAAGTACTATGCAGACTCCGTGAAGGGCCGATT |
| | | | CACCATCTCCAGAGACAATTCCAAGAACACGCTGTATCTGC |
| | | | AAATGAACAGCCTGAGAGCTGAGGACACGGCTATGTATTA |
| | | | CTGTGCGAAAGATCGGATTTCCTATAACTGGAACCTTGACT |
| | | | ACTGGGGCCAGGGAACCCTGGTCACCGTCTCCTCA |
| | SEQ ID | HCDR1 | NYGMH |
| | NO: 16 | (Kabat) | |
| | SEQ ID | HCDR2 | VISYDGVDKYYADSVKG |
| | NO: 17 | (Kabat) | |
| | SEQ ID | HCDR3 | DRISYNWNLDY |
| | NO: 18 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab02110 | SEQ ID | VH (AA) | QVQLVESGGGVVQPGRSLRLSCVASGFTFSNYGLHWVRQAP |
| | NO: 19 | | GKGLEWVAAISYDGSDKYYADPVKGRFTISRDTTKNTLSLQM |
| | | | NSLRAEDTAVYYCAKERYRGYDWDFDYWGQGTLVTVSS |
| | SEQ ID | VH (DNA) | CAGGTGCAGTTGGTGGAGTCTGGGGGAGGCGTGGTCCAGC |
| | NO: 20 | | CTGGGAGGTCCCTGAGACTCTCCTGTGTAGCCTCTGGATTC |
| | | | ACCTTCAGTAACTATGGCCTACACTGGGTCCGCCAGGCTCC |
| | | | AGGCAAGGGGCTGGAGTGGGTGGCAGCTATATCATATGAT |
| | | | GGAAGTGATAAATACTATGCAGACCCCGTGAAGGGCCGAT |
| | | | TCACCATCTCCAGAGACACGACCAAGAACACGCTGTCTCTG |
| | | | CAAATGAACAGTCTGAGAGCTGAGGACACGGCTGTGTATT |
| | | | ACTGTGCGAAAGAAAGATATCGTGGCTACGATTGGGACTTT |
| | | | GACTACTGGGGCCAGGGAACCCTGGTCACCGTCTCCTCA |
| | SEQ ID | HCDR1 | NYGLH |
| | NO: 21 | (Kabat) | |
| | SEQ ID | HCDR2 | AISYDGSDKYYADPVKG |
| | NO: 22 | (Kabat) | |
| | SEQ ID | HCDR3 | ERYRGYDWDFDY |
| | NO: 23 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab15210 | SEQ ID | VH (AA) | QVQLVESGGGVVQPGRSLRLSCAASGFTFSSYGLHWVRQAPG |
| | NO: 24 | | KGLEWVAVISFDGSNKYYADSVKGRFTISRDTSKNTLSLQMN |
| | | | SLRAEDTAVYYCAKERSRGYDWDFDYWGQGILVTVSS |
| | SEQ ID | VH (DNA) | CAGGTGCAGCTGGTGGAGTCTGGGGGAGGCGTGGTCCAGC |
| | NO: 25 | | CTGGGAGGTCCCTGAGACTCTCCTGTGCAGCCTCTGGATTC |
| | | | ACCTTCAGTAGCTATGGCCTACACTGGGTCCGCCAGGCTCC |
| | | | AGGCAAGGGGCTGGAGTGGGTGGCAGTTATATCATTTGAT |
| | | | GGAAGTAATAAATACTATGCAGACTCCGTGAAGGGCCGAT |
| | | | TCACCATCTCCAGAGACACTTCCAAGAACACGCTGTCTCTG |
| | | | CAAATGAACAGCCTGAGAGCTGAGGACACGGCTGTGTATT |
| | | | ACTGTGCGAAAGAAAGATCTCGTGGCTACGATTGGGACTTT |
| | | | GACTACTGGGGCCAGGGAATCCTGGTCACCGTCTCCTCA |
| | SEQ ID | HCDR1 | SYGLH |
| | NO: 26 | (Kabat) | |
| | SEQ ID | HCDR2 | VISFDGSNKYYADSVKG |
| | NO: 27 | (Kabat) | |
| | SEQ ID | HCDR3 | ERSRGYDWDFDY |
| | NO: 28 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 062Ab16310 | SEQ ID | VH (AA) | QVRLVESGGGVVQPGRSLRLSCAASGFTFSNYGMHWVRQAP |
| | NO: 29 | | GKGLEWVAVISYDGSDKYYPDSVKGRFTISRDNSRNTLYLQM |
| | | | NSLSTEDTAVYYCAKDQYRGYDGTFDYWGQGTLVTVSS |
| | SEQ ID | VH (DNA) | CAGGTGCGGCTGGTGGAGTCGGGGGGAGGCGTGGTCCAGC |
| | NO: 30 | | CTGGGAGGTCCCTGAGACTCTCCTGTGCAGCCTCTGGATTC |
| | | | ACCTTCAGTAACTATGGCATGCACTGGGTCCGCCAGGCTCC |
| | | | AGGCAAGGGGCTGGAGTGGGTGGCAGTTATATCATATGAT |
| | | | GGAAGTGATAAGTACTATCCAGACTCCGTGAAGGGCCGCTT |
| | | | CACCATCTCCAGAGACAATTCCAGAAACACGCTGTATCTGC |
| | | | AAATGAACAGCCTGAGTACTGAGGACACGGCTGTTTATTAC |
| | | | TGTGCGAAAGATCAATATCGTGGCTACGATGGGACTTTTGA |
| | | | CTACTGGGGCCAGGGAACCCTGGTCACCGTCTCCTCA |
| | SEQ ID | HCDR1 | NYGMH |
| | NO: 31 | (Kabat) | |
| | SEQ ID | HCDR2 | VISYDGSDKYYPDSVKG |
| | NO: 32 | (Kabat) | |
| | SEQ ID | HCDR3 | DQYRGYDGTFDY |
| | NO: 33 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab05510 | SEQ ID | VH (AA) | QVQLVESGGGVVQPGRSLRLSCAASGFIFSSYNMHWVRQAPG |
| | NO: 34 | | KGLEWVAVISYDRVNKYYADSVKGRFTISRDNSKNTLYLQM |
| | | | NSLRVEDTAVYYCAKERERYFDWLDYWGQGTLVTVSS |
| | SEQ ID | VH (DNA) | CAGGTGCAGCTGGTGGAGTCTGGGGGAGGCGTGGTCCAGC |
| | NO: 35 | | CTGGGAGGTCCCTGAGACTCTCCTGTGCAGCCTCTGGATTC |
| | | | ATCTTCAGTAGCTATAACATGCACTGGGTCCGCCAGGCTCC |
| | | | AGGCAAGGGGCTGGAGTGGGTGGCAGTTATATCATATGAT |
| | | | AGAGTTAATAAATACTATGCAGACTCCGTGAAGGGCCGATT |
| | | | CACCATCTCCAGAGACAATTCCAAGAACACGCTGTATCTGC |
| | | | AAATGAACAGCCTGAGAGTTGAGGACACGGCTGTGTATTA |
| | | | CTGTGCGAAAGAAAGGGAACGATATTTTGACTGGTTGGACT |
| | | | ACTGGGGCCAGGGAACCCTGGTCACCGTCTCCTCA |
| | SEQ ID | HCDR1 | SYNMH |
| | NO: 36 | (Kabat) | |
| | SEQ ID | HCDR2 | VISYDRVNKYYADSVKG |
| | NO: 37 | (Kabat) | |
| | SEQ ID | HCDR3 | ERERYFDWLDY |
| | NO: 38 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab07710 | SEQ ID | VH (AA) | QVQLVESGGGVVQPGRSLRLSCAASGFTFSSYGMHWVRQAP |
| | NO: 39 | | GKGLEWVAVISNDGSYKYYVDSVTGRFTISRDNSKNTLYLQM |
| | | | NSLRAEDTAVYYCAKDRNMGYSGWFDYWGQGSLVTVSS |
| | SEQ ID | VH (DNA) | CAGGTGCAGCTGGTGGAGTCTGGGGGAGGCGTGGTCCAGC |
| | NO: 40 | | CTGGGAGGTCCCTGAGACTCTCCTGTGCAGCCTCTGGATTC |
| | | | ACCTTCAGTAGCTATGGCATGCACTGGGTCCGCCAGGCTCC |
| | | | AGGCAAGGGGCTGGAGTGGGTGGCAGTTATATCAAATGAT |
| | | | GGAAGTTATAAATACTATGTAGACTCCGTGACGGGCCGATT |
| | | | CACCATCTCCAGAGACAATTCCAAGAACACGCTGTATCTGC |
| | | | AAATGAACAGCCTGAGAGCTGAGGACACGGCTGTGTATTA |
| | | | CTGTGCGAAAGATCGGAATATGGGATATAGTGGCTGGTTTG |
| | | | ACTACTGGGGCCAGGGATCCCTGGTCACCGTCTCCTCA |
| | SEQ ID | HCDR1 | SYGMH |
| | NO: 41 | (Kabat) | |
| | SEQ ID | HCDR2 | VISNDGSYKYYVDSVTG |
| | NO: 42 | (Kabat) | |
| | SEQ ID | HCDR3 | DRNMGYSGWFDY |
| | NO: 43 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab14710 | SEQ ID | VH (AA) | QVQLVESGGGVVQPGRSLRLSCAASGFTFSSHGMHWVRQAP |
| | NO: 44 | | GKGLEWVAVISNDGSYKYYADSVTGRFTISRDNSKNTLYLQM |
| | | | NSLRAEDTAVYYCAKDRNMGYSGWFDYWGQGSLVTVSS |
| | SEQ ID | VH (DNA) | CAGGTGCAGCTGGTGGAGTCTGGGGGAGGCGTGGTCCAGC |
| | NO: 45 | | CTGGGAGGTCCCTGAGACTCTCCTGTGCAGCCTCTGGATTC |
| | | | ACCTTCAGTAGCCATGGCATGCACTGGGTCCGCCAGGCTCC |
| | | | AGGCAAGGGGCTGGAGTGGGTGGCAGTTATATCAAATGAT |
| | | | GGAAGTTATAAATACTATGCAGACTCCGTGACGGGCCGATT |
| | | | CACCATCTCCAGAGACAATTCCAAGAACACGCTGTATCTGC |
| | | | AAATGAACAGCCTGAGAGCTGAGGACACGGCTGTGTATTA |
| | | | CTGTGCGAAAGATCGGAATATGGGATACAGTGGCTGGTTTG |
| | | | ACTACTGGGGCCAGGGATCCCTGGTCACCGTCTCCTCA |
| | SEQ ID | HCDR1 | SHGMH |
| | NO: 46 | (Kabat) | |
| | SEQ ID | HCDR2 | VISNDGSYKYYADSVTG |
| | NO: 47 | (Kabat) | |
| | SEQ ID | HCDR3 | DRNMGYSGWFDY |
| | NO: 48 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 061Ab05110 | SEQ ID | VH (AA) | QVQLVESGGGVVQPGRSLRLSCAASGFTFSNYGMHWVRQAP |
| | NO: 49 | | GKGLEWVAVISNDGSEKYYADSVKGRFTISRDSSKNTLYLQM |
| | | | NSLRAEDTAVYYCAKAPWGSGTTGRLDYWGQGTLVTVSS |
| | SEQ ID | VH (DNA) | CAGGTGCAGCTGGTGGAGTCTGGGGGAGGCGTGGTCCAGC |
| | NO: 50 | | CTGGGAGGTCCCTGAGACTCTCCTGTGCAGCCTCTGGATTC |
| | | | ACCTTCAGTAACTATGGCATGCACTGGGTCCGCCAGGCTCC |
| | | | AGGCAAGGGGCTGGAGTGGGTGGCTGTTATATCAAATGAT |
| | | | GGAAGTGAAAAATACTATGCAGACTCCGTGAAGGGCCGAT |
| | | | TCACCATCTCCAGAGACAGTTCCAAGAACACGCTCTATCTA |
| | | | CAGATGAACAGCCTGAGAGCTGAGGACACGGCTGTGTATT |
| | | | ACTGTGCGAAAGCCCCTTGGGGAAGTGGAACGACGGGGAG |
| | | | GCTTGACTACTGGGGCCAGGGAACCCTGGTCACCGTCTCCT |
| | | | CA |
| | SEQ ID | HCDR1 | NYGMH |
| | NO: 51 | (Kabat) | |
| | SEQ ID | HCDR2 | VISNDGSEKYYADSVKG |
| | NO: 52 | (Kabat) | |
| | SEQ ID | HCDR3 | APWGSGTTGRLDY |
| | NO: 53 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab03210 | SEQ ID | VH (AA) | QFQLQESGPGLVKPSETLSLTCTVSGGSISTSSYYWGWIRQPPG |
| | NO: 54 | | KGLEWIGTIYYSGTTYYNPSLKSRVTISVDTSKNQFSLKLKSVT |
| | | | AADTSIYYCARHWPKYNSWFDPWGQGILVTVSS |
| | SEQ ID | VH (DNA) | CAATTTCAGCTACAGGAGTCGGGCCCAGGACTGGTGAAGC |
| | NO: 55 | | CTTCGGAGACCCTGTCCCTCACCTGCACTGTCTCTGGTGGCT |
| | | | CCATCAGCACTAGTAGTTACTACTGGGGCTGGATCCGCCAG |
| | | | CCCCCAGGGAAGGGGCTGGAGTGGATTGGGACTATCTATT |
| | | | ATAGTGGGACCACCTACTACAACCCGTCCCTCAAGAGTCGA |
| | | | GTCACCATATCCGTAGACACGTCCAAGAACCAGTTCTCCCT |
| | | | GAAGCTGAAGTCTGTGACCGCCGCAGACACGTCTATCTATT |
| | | | ACTGTGCGAGACATTGGCCCAAGTACAACTCCTGGTTCGAC |
| | | | CCCTGGGGCCAGGGAATTCTGGTCACCGTCTCCTCA |
| | SEQ ID | HCDR1 | TSSYYWG |
| | NO: 56 | (Kabat) | |
| | SEQ ID | HCDR2 | TIYYSGTTYYNPSLKS |
| | NO: 57 | (Kabat) | |
| | SEQ ID | HCDR3 | HWPKYNSWFDP |
| | NO: 58 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| 063Ab16720 | SEQ ID | VH (AA) | QFQLQESGPGLVKPSETLSLTCTVSGGSISTSSYYWGWIRQPPG |
| | NO: 59 | | KGLEWIGTIYFSGTTYYNPSLTSRVSMSVDTSKNQFSLKLNSV |
| | | | TAADTSIYYCARHWPKYNSWFDPWGQGFLVTVSS |
| | SEQ ID | VH (DNA) | CAATTTCAACTACAGGAGTCGGGCCCAGGACTGGTGAAGC |
| | NO: 60 | | CTTCGGAGACCCTGTCCCTCACCTGCACTGTCTCTGGTGGCT |
| | | | CCATCAGCACTAGTAGTTACTACTGGGGCTGGATCCGCCAG |
| | | | CCCCCAGGGAAGGGGCTGGAGTGGATTGGGACTATCTATTT |
| | | | TAGTGGGACCACCTACTACAACCCGTCCCTCACGAGTCGAG |
| | | | TCAGCATGTCCGTAGACACGTCCAAGAACCAGTTCTCCCTG |
| | | | AAGCTGAACTCTGTGACCGCCGCAGACACGTCTATCTATTA |
| | | | CTGTGCGAGACATTGGCCCAAGTACAACTCCTGGTTCGACC |
| | | | CCTGGGGCCAGGGATTTCTGGTCACCGTCTCCTCA |
| | SEQ ID | HCDR1 | TSSYYWG |
| | NO: 61 | (Kabat) | |
| | SEQ ID | HCDR2 | TIYFSGTTYYNPSLTS |
| | NO: 62 | (Kabat) | |
| | SEQ ID | HCDR3 | HWPKYNSWFDP |
| | NO: 63 | (Kabat) | |
| | SEQ ID | VL (AA) | EIVLTQSPATLSLSPGERATLSCRASQSVSSYLAWYQQKPGQA |
| | NO: 64 | | PRLLIYDASNRATGIPARFSGSGSGTDFTLTISSLEPEDFAVYYC |
| | | | QQRSNWPPTFGQGTKVEIK |
| | SEQ ID | VL (DNA) | GAAATTGTGTTGACACAGTCTCCAGCCACCCTGTCTTTGTCT |
| | NO: 65 | | CCAGGGGAAAGAGCCACCCTCTCCTGCAGGGCCAGTCAGA |
| | | | GTGTTAGTAGTTACTTAGCCTGGTACCAACAGAAACCTGGC |
| | | | CAGGCTCCCAGGCTCCTCATCTATGATGCATCCAACAGGGC |
| | | | CACTGGCATCCCAGCCAGGTTCAGTGGCAGTGGGTCTGGGA |
| | | | CAGACTTCACTCTCACCATCAGCAGCCTAGAGCCTGAAGAT |
| | | | TTTGCAGTTTATTACTGTCAGCAGCGTAGCAACTGGCCTCC |
| | | | GACGTTCGGCCAAGGGACCAAGGTGGAAATCAAA |
| | SEQ ID | LCDR1 | RASQSVSSYLA |
| | NO: 66 | (Kabat) | |
| | SEQ ID | LCDR2 | DASNRAT |
| | NO: 67 | (Kabat) | |
| | SEQ ID | LCDR3 | QQRSNWPPT |
| | NO: 68 | (Kabat) | |
| human | SEQ ID | AA | ASTKGPSVFPLAPSSKSTSGGTAALGCLVKDYFPEPVTVSWNS |
| IgG1 | NO: 70 | | GALTSGVHTFPAVLQSSGLYSLSSVVTVPSSSLGTQTYICNVNH |
| constant | | | KPSNTKVDKKVEPKSCDKTHTCPPCPAPELLGGPSVFLFPPKP |
| region | | | KDTLMISRTPEVTCVVVDVSHEDPEVKFNWYVDGVEVHNAK |
| | | | TKPREEQYNSTYRVVSVLTVLHQDWLNGKEYKCKVSNKALP |
| | | | APIEKTISKAKGQPREPQVYTLPPSREEMTKNQVSLTCLVKGFY |
| | | | PSDIAVEWESNGQPENNYKTTPPVLDSDGSFFLYSKLTVDKSR |
| | | | WQQGNVFSCSVMHEALHNHYTQKSLSLSPGK |
| human | SEQ ID | AA | RTVAAPSVFIFPPSDEQLKSGTASVVCLLNNFYPREAKVQWKV |
| kappa | NO: 71 | | DNALQSGNSQESVTEQDSKDSTYSLSSTLTLSKADYEKHKVY |
| chain | | | ACEVTHQGLSSPVTKSFNRGEC |
| constant | | | |
| region | | | |
| Variant of | SEQ ID | AA | ASTKGPSVFPLAPSSKSTSGGTAALGCLVKDYFPEPVTVSWNS |
| human | NO: 95 | | GALTSGVHTFPAVLQSSGLYSLSSVVTVPSSSLGTQTYICNVNH |
| IgG1 | | | KPSNTKVDKKVEPKSCDKTHTCPPCPAPELLGGPSVFLFPPKP |
| constant | | | KDTLMISRTPEVTCVVVDVSHEDPEVKFNWYVDGVEVHNAK |
| region | | | TKPREEQYNSTYRVVSVLTVLHQDWLNGKEYKCKVSNKALP |
| with Knob | | | APIEKTISKAKGQPREPQVYTLPPSREEMTKNQVSLWCLVKGF |
| mutation | | | YPSDIAVEWESNGQPENNYKTTPPVLDSDGSFFLYSKLTVDKS |
| T366W | | | RWQQGNVFSCSVMHEALHNHYTQKSLSLSPGK |
| Variant of | SEQ ID | AA | ASTKGPSVFPLAPSSKSTSGGTAALGCLVKDYFPEPVTVSWNS |
| human | NO: 96 | | GALTSGVHTFPAVLQSSGLYSLSSVVTVPSSSLGTQTYICNVNH |
| IgG1 | | | KPSNTKVDKKVEPKSCDKTHTCPPCPAPELLGGPSVFLFPPKP |
| constant | | | KDTLMISRTPEVTCVVVDVSHEDPEVKFNWYVDGVEVHNAK |
| region | | | TKPREEQYNSTYRVVSVLTVLHQDWLNGKEYKCKVSNKALP |
| with hole | | | APIEKTISKAKGQPREPQVYTLPPSREEMTKNQVSLSCAVKGF |
| mutations | | | YPSDIAVEWESNGQPENNYKTTPPVLDSDGSFFLVSKLTVDKS |
| T366S, | | | RWQQGNVFSCSVMHEALHNHYTQKSLSLSPGK |
| L368A, | | | |
| and | | | |
| Y407V | | | |

### Example 3. Determination of the Binding Affinity and Specificity of Anti-cMET Antibodies

The binding affinity and specificity of purified anti-cMET antibodies were determined by ELISA and FACS. Briefly, 2 mg/ml of human or cyno monomer cMET fusion protein human cMET-ECD-his or cyno cMET-ECD-his (in house generated, SEQ ID Nos: 1 and 3) or human dimer cMET protein formed by human cMET-ECD-mFc (in house generated, SEQ ID NO: 2) was coated in the 96 well ELISA plates, and 50 mL of serially diluted antibodies were co-incubated for 30-60 min, washed, and incubated with goat anti-human IgG secondary antibody conjugated to HRP (Abcam, ab98624). After incubation and washing, the plates were developed with HRP substrate and absorbance was measured. Candidates positive for ELISA binding were serially diluted and incubated with Hs746T cells, a human gastric carcinoma line which has a putative oncogenic mutation with amplification of cMET resulting in high cMET expression, for 30 minutes at 4° C. After washing twice with FACS buffer, diluted Alexa Fluor 647 goat anti-human IgG secondary antibody was added and incubated with Hs746T cells for 30 minutes at 4° C. in the dark. After washing twice with FACS buffer, cells were resuspended with FACS buffer and read using a Becton Dickinson LSR Fortessa™ cell analyzer. Non-specific bindings of these antibodies were evaluated by FACS binding against Jurkat cells (cMET negative cells). Titration curves were generated using a sigmoidal dose-response of nonlinear fit from GraphPad™ Software by Dotmatics.

As shown in Table 7 and Table 8, all selected candidates specifically bind to cMET monomer and dimer protein in high affinity, and they all specifically bind to human cMET expressing cancer cells and cross bind to cyno cMET.

TABLE 7
Binding affinity of anti-cMET antibodies
against human and cyno cMET protein

| | EC50 against | EC50 against | EC50 against |
|---|---|---|---|
| | human cMET | cyno cMET | humanc MET |
| Antibodies | monomer (nM) | momoner (nM) | dimer (nM) |
| 063Ab10910 | 0.01741 | 0.009868 | 0.0543 |
| 061Ab15310 | 0.07772 | 0.1846 | 0.3797 |
| 063Ab16010 | 0.1113 | 0.04456 | 0.6866 |
| 063Ab02110 | 0.02074 | 0.009973 | 0.0705 |
| 063Ab15210 | 0.06627 | 0.05644 | 0.8757 |
| 062Ab16310 | 0.1269 | 0.09184 | 0.04956 |
| 063Ab05510 | 0.5898 | 0.5662 | 2.364 |
| 063Ab07710 | 0.2169 | 0.2971 | 1.346 |
| 063Ab14710 | 0.1453 | 0.1078 | 0.5235 |
| 061Ab05110 | 0.573 | 4.264 | 0.2883 |
| 063Ab03210 | 0.03022 | 0.01385 | 0.05504 |
| 063Ab16720 | 0.02154 | 0.01365 | 0.0362 |

TABLE 8
Binding affinity of anti-cMET antibodies against Hs746T cells and Jurkat cells

| | EC50 against | Emax against | EC50 against | Emax against |
|---|---|---|---|---|
| | Hs746T cell line | Hs746T cell line | Jurkat cell line | Jurkat cell line |
| Antibodies | (nM) | (MFI) | (EC50) | (MFI) |
| 063Ab10910 | 0.5171 | 6.30E+06 | n.d. | 1.06E+04 |
| 061Ab15310 | 0.6303 | 5.91E+06 | n.d. | 3.48E+03 |
| 063Ab16010 | 0.638 | 6.31E+06 | n.d. | 1.02E+04 |
| 063Ab02110 | 0.3061 | 5.83E+06 | n.d. | 1.25E+04 |
| 063Ab15210 | 0.5425 | 5.77E+06 | n.d. | 8.51E+03 |
| 062Ab16310 | 0.2851 | 4.39E+06 | n.d. | 3.60E+03 |
| 063Ab05510 | 0.2497 | 5.07E+06 | n.d. | 4.78E+03 |
| 063Ab07710 | 1.215 | 4.89E+06 | n.d. | 8.36E+03 |
| 063Ab14710 | 1.252 | 5.93E+06 | n.d. | 1.24E+04 |
| 061Ab05110 | 0.9413 | 5.39E+06 | n.d. | 3.57E+03 |
| 063Ab03210 | 0.4364 | 3.22E+06 | n.d. | 6.20E+03 |
| 063Ab16720 | 0.4065 | 4.16E+06 | n.d. | 5.90E+03 |
| hIgG | n.d. | 2.75E+03 | n.d. | 3.78E+03 |

n.d.: non detected

### Example 4. Binding Kinetics of Anti-cMET Antibodies

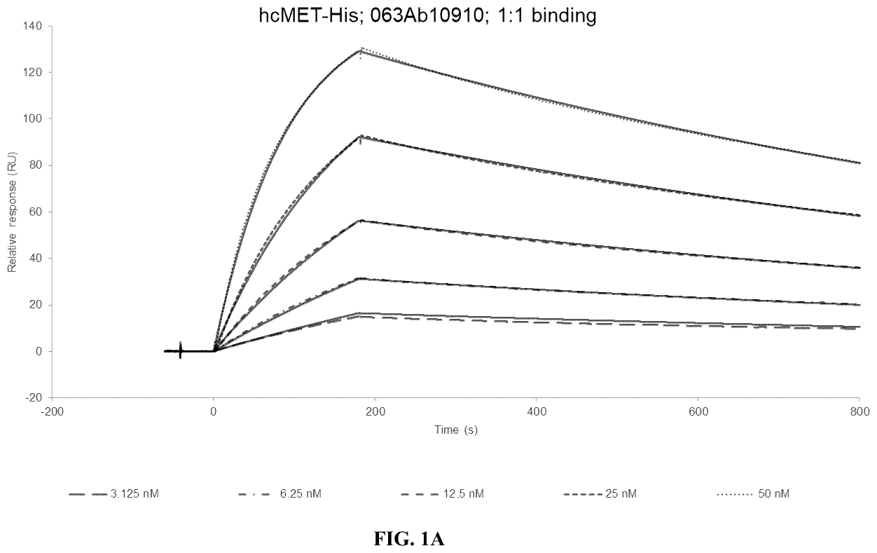

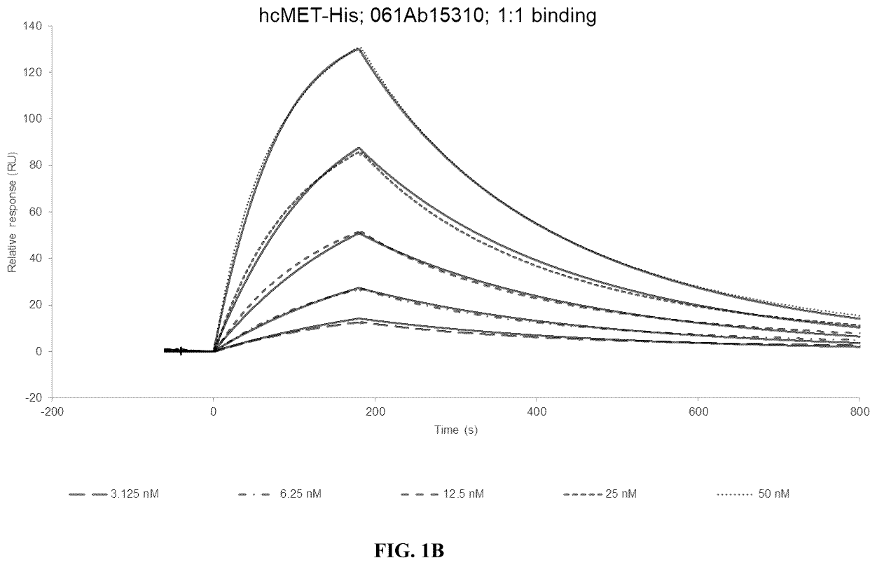

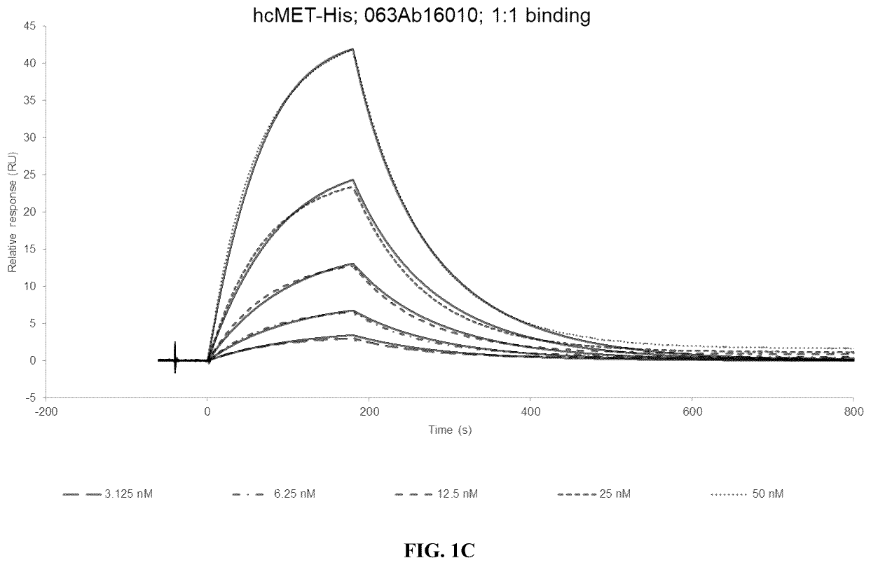

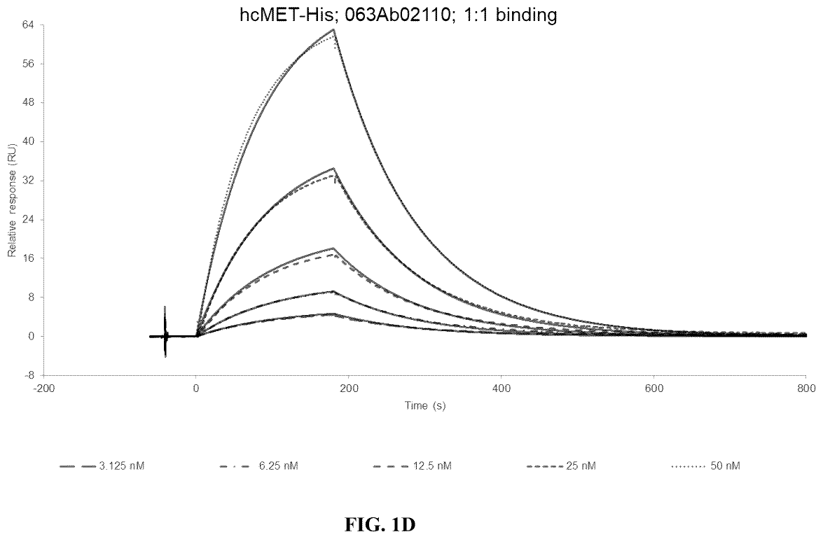

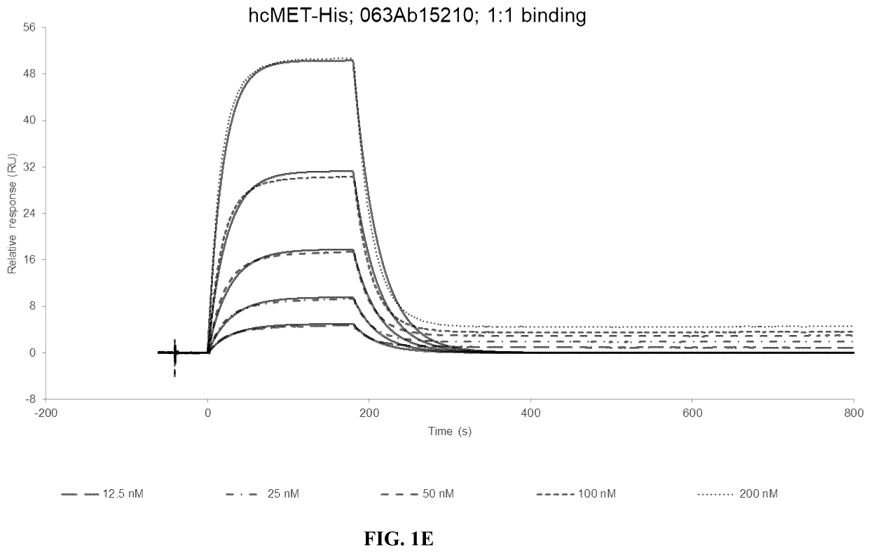

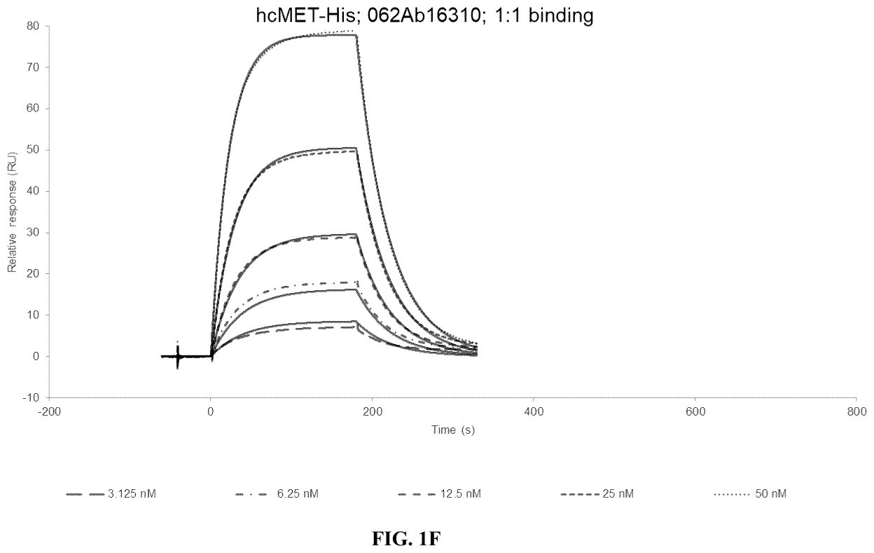

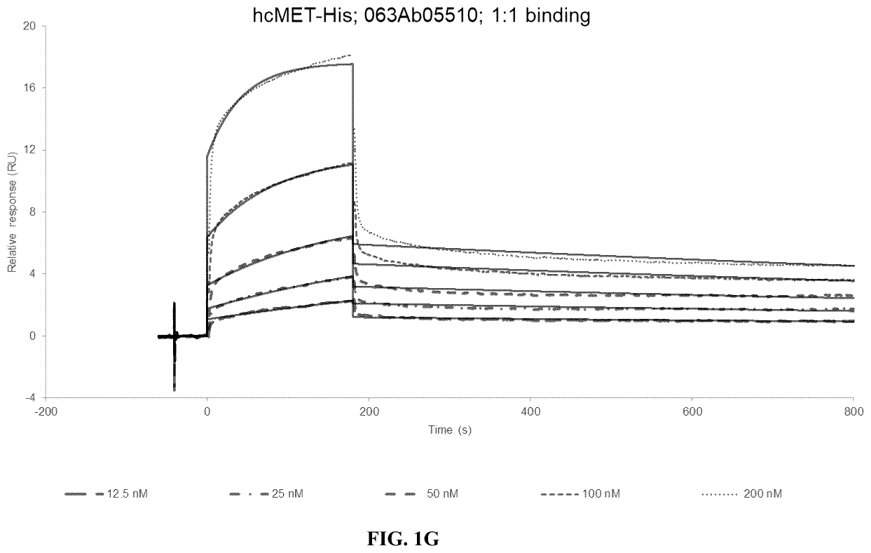

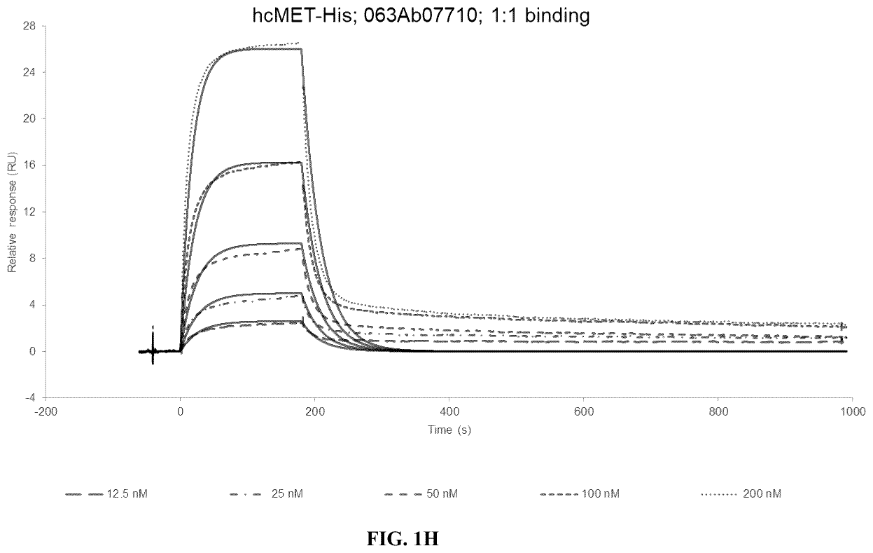

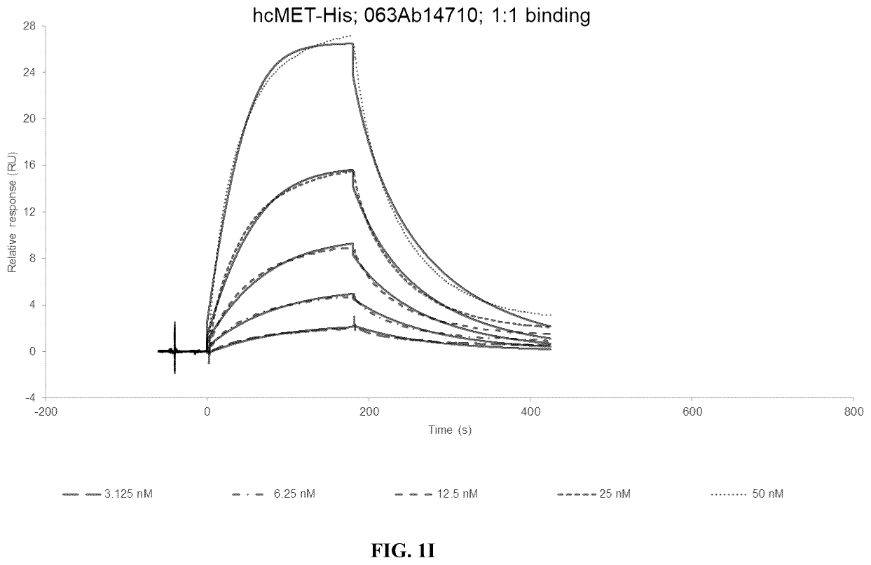

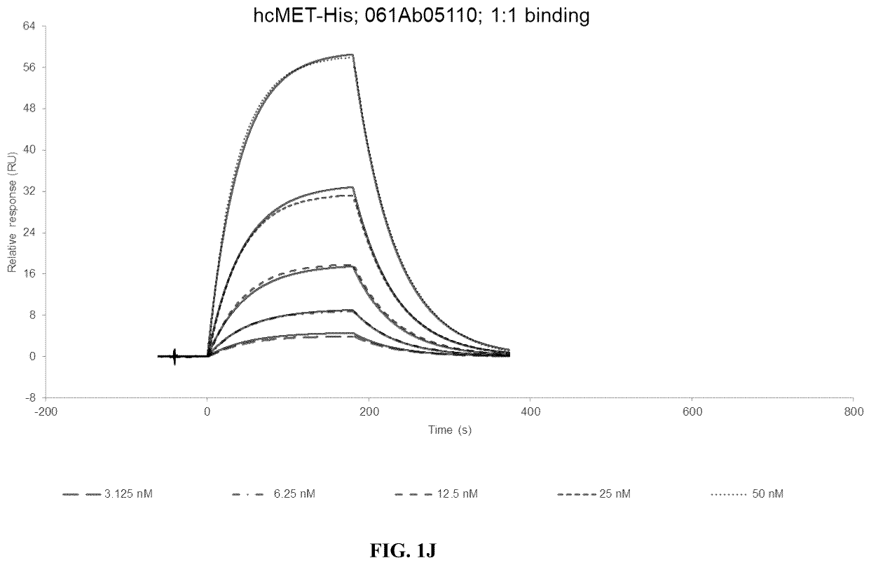

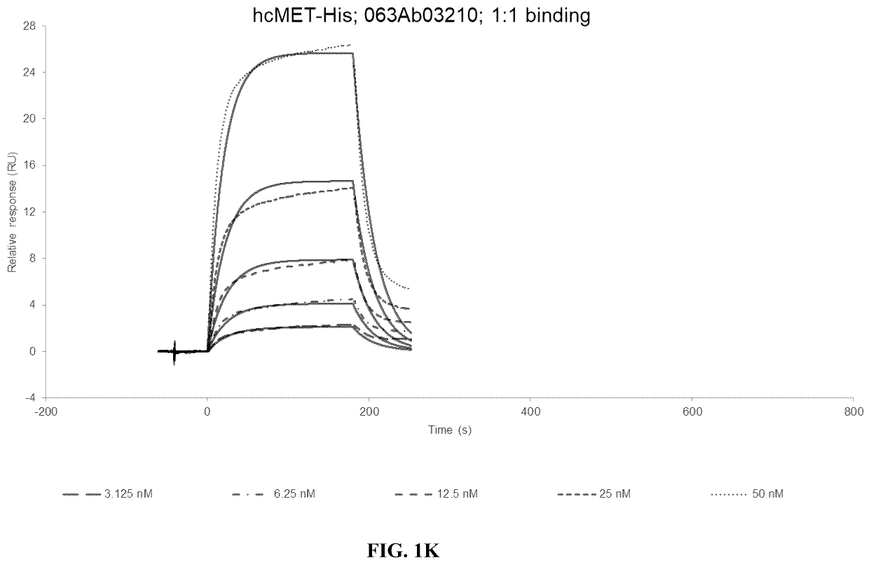

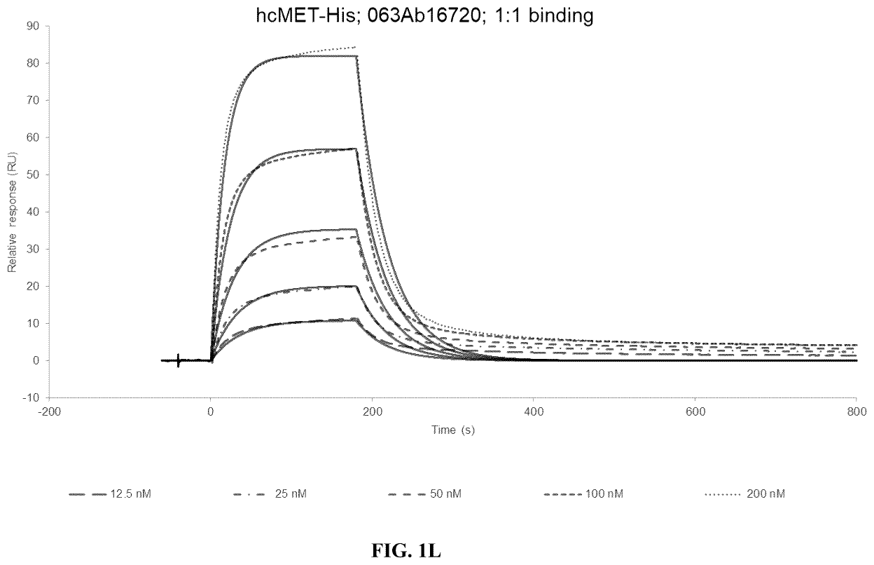

The binding affinity and kinetics of purified anti-cMET antibodies were determined by Surface plasmon resonance (Biacore 8K, GE Life Sciences) at room temperature. Briefly, mouse anti-human IgG Fc antibody was immobilized via amine coupling onto a activated CM5 biosensor chip (Cat. No. BR100530, GE Life Sciences). Purified monoclonal antibody candidates were flowed over the chip surface and captured by anti-human IgG antibody. Then a serial dilution of soluble monomeric human cMET fusion proteins with his-tag (in house generated, SEQ ID NO: 1) were injected over the antibodies captured surface and changes in surface plasmon resonance signals were analyzed to calculate the association rates (kₒₙ) and dissociation rates (k_{off}) by using the one-to-one Langmuir binding model (BIA Evaluation Software™, GE Life Sciences). The equilibrium dissociation constant (K_{D}) was calculated as the ratio k_{off}/kₒₙ. The binding affinity and kinetics profiles of selected monoclonal anti-cMET antibodies are listed below in Table 9, and the detail binding curves of selected candidates are presented in FIGS. 1A-1L.

TABLE 9
Binding kinetics of anti-cMET antibodies
against monomeric cMET protein

| | 1:1 binding ka | | |
|---|---|---|---|
| Antibodies | (1/Ms) | kd (1/s) | KD (M) |
| 063Ab10910 | 2.28E+05 | 7.78E−04 | 3.40E−09 |
| 061Ab15310 | 3.06E+05 | 5.35E−03 | 1.75E−08 |
| 063Ab16010 | 6.30E+05 | 5.06E−02 | 8.04E−08 |
| 063Ab02110 | 6.14E+04 | 9.50E−03 | 1.55E−07 |
| 063Ab15210 | 1.03E+05 | 3.22E−02 | 3.11E−07 |
| 062Ab16310 | 4.55E+05 | 2.66E−02 | 5.85E−08 |
| 063Ab05510 | 1.38E+05 | 2.35E−02 | 1.70E−07 |
| 063Ab07710 | 1.00E+05 | 3.63E−02 | 3.62E−07 |
| 063Ab14710 | 1.56E+06 | 5.02E−02 | 3.23E−08 |
| 061Ab05110 | 1.17E+05 | 1.96E−02 | 1.68E−07 |
| 063Ab03210 | 2.03E+05 | 3.40E−02 | 1.68E−07 |
| 063Ab16720 | 1.65E+05 | 2.59E−02 | 1.56E−07 |

### Example 5. Blocking Activity on Ligand-Induced Signaling of Monoclonal Anti-cMET Antibodies

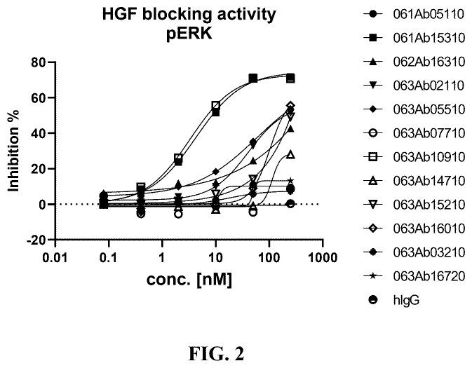

To assess the activity of anti-cMET antibodies to interfere with the HGF-cMET interaction and inhibit the downstream signaling, the inhibition of HGF-induced ERK phosphorylation at T202/Y204 upon antibody treatment was used as a cellular readout. Briefly, H596 cells (epithelial-like cell line isolated from human lung cancer) were seeded to one 96-well assay plate and incubated at 37° C., 5% C02. After 24 hours, cells were starved with medium without FBS for 4 hours. Then, a series of dilutions of anti-cMET antibodies were added to each well and incubated at 37° C. for 1 hour. 100 pM human HGF was added to each well and incubated at 37° C. for 15 minutes. Then, the medium was discarded and 1×lysis buffer was added to the assay plate and incubated at room temperature for 30 minutes. Cellular phosphor-ERK was measured using ERK phospho-T202/Y204 Kit (PerkinElmer). IC50 values were determined by fitting the dose-response data to the four-parameter logistic model with GraphPad Prism. Imax was calculated using the equation: % inhibition=100*[1−(X−MIN)/(MAX−MIN)]. X is the FRET signal at a given compound concentration. MAX is the signal in the presence of H596 cells and 100 pM hHGF. MIN is the signal in the presence of starvation medium as the background signal. IC50 and Imax of monoclonal anti-cMET antibodies are shown in Table 10 and FIG. 2.

TABLE 10
Comparison of blocking activity on ligand-induced
signaling of monoclonal anti-cMET antibodies

| Antibody | IC50 (nM) | Imax (%) |
|---|---|---|
| 061Ab05110 | 11.75 | 10.84 |
| 061Ab15310 | 4.26 | 71.60 |
| 062Ab16310 | 65.62 | 42.74 |
| 063Ab02110 | 40.75 | 51.40 |
| 063Ab05510 | 58.02 | 53.66 |
| 063Ab07710 | >250 | 8.71 |
| 063Ab10910 | 3.30 | 70.95 |
| 063Ab14710 | 113.00 | 28.06 |
| 063Ab15210 | 65.73 | 49.19 |
| 063Ab16010 | 102.90 | 55.64 |
| 063Ab03210 | >250 | 7.41 |
| 063Ab16720 | 9.57 | 13.20 |
| hIgG | N/A | 0.33 |

### Example 6. Agonist Activity of Monoclonal Anti-cMET Antibodies

To assess the transient agonistic activity of anti-cMET antibodies, the activation of cMET downstream signaling: upregulation of ERK phospho-T202/Y204 was measured upon antibody treatment. Briefly, H596 cells were seeded to the 96-well assay plate and incubated at 37° C., 5% C02. After 24 hours, cells were starved with medium without FBS for 4 hours. Then, 50 nM of anti-cMET antibodies were added to each well and incubated at 37° C. for 2 hours. Then, the medium was discarded and 1×lysis buffer was added to the assay plate and incubated at room temperature for 30 minutes. Cellular phosphor-ERK was measured using ERK phospho-T202/Y204 Kit (PerkinElmer). Stimulation fold was determined by comparing the p-ERK signal in the presence of 50 nM antibody with that in the presence of the vehicle (buffer PBS). The agonist activity of monoclonal anti-cMET antibodies are shown in Table 11.

TABLE 11
Agonist activity of monoclonal anti-cMET antibodies

| Antibody | Stimulation fold at 50 nM |
|---|---|
| 061Ab05110 | 3.43 |
| 061Ab15310 | 4.06 |
| 062Ab16310 | 5.47 |
| 063Ab02110 | 2.88 |
| 063Ab05510 | 3.50 |
| 063Ab07710 | 4.57 |
| 063Ab10910 | 1.21 |
| 063Ab14710 | 3.62 |
| 063Ab15210 | 3.66 |
| 063Ab16010 | 4.09 |
| 063Ab03210 | 5.72 |
| 063Ab16720 | 5.18 |
| hIgG | 0.96 |

### Example 7. Anti-cMET Antibodies Bind to Different Epitopes

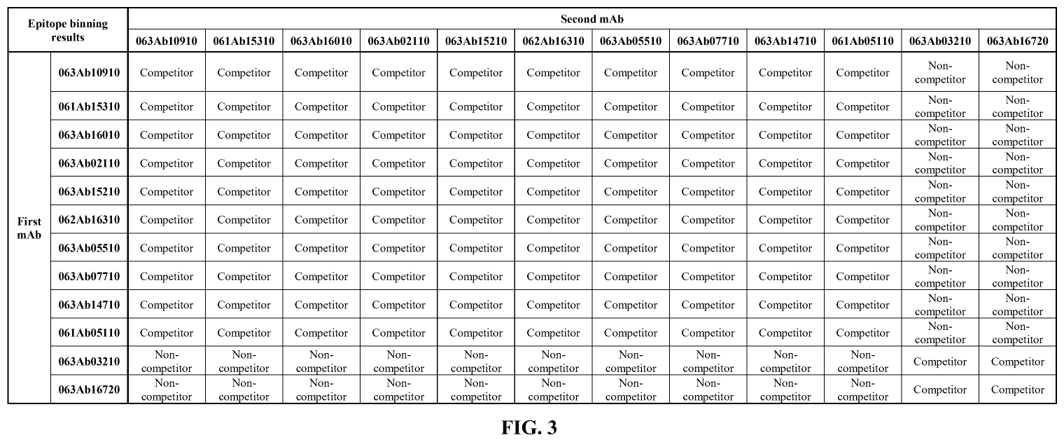

To assess whether anti-cMET antibodies compete with each other, an epitope binning assay was performed using Surface plasmon resonance (Biacore 8K, GE Life Sciences) at room temperature. Briefly, human cMET-his (in house generated, SEQ ID NO: 1) is captured to the sensor surface of anti-his antibody immobilized CM5 chip (Cat. No. 29234602, GE Life Sciences). The first antibody was injected at 300 nM to saturate antigen binding, followed by the injection of the second antibodies. Data was analyzed utilizing Biacore Insight Epitope Binning Extension. Epitope binning results of representative monoclonal antibodies are listed in FIG. 3. Based on FIG. 3, the Anti-cMET antibodies are divided into two non-competitive groups: (1) 063Ab10910, 061Ab15310, 063Ab16010, 063Ab02110, 063Ab15210, 062Ab16310, 063Ab05510, 063Ab07710, 063Ab14710 or 061Ab05110; and (2) 063Ab03210, or 063Ab16720.

### Example 8. Construction of Bispecific Antibodies have 2 Different Antigen Binding Domains Against Distinct Epitopes of cMET

In order to generate bi-paratopic antibodies against cMET, we constructed bispecific antibodies comprising two different arms (Arm1 and Arm2), wherein Arm1 and Arm2 are derived from different anti-cMET monospecific antibodies which bind to separate epitopes and are non-competitive.

The individual anti-cMET monospecific antibodies were derived from candidates described in Examples 1 to 7 herein. All anti-cMET antibodies described here share the common light chain (SEQ ID NO: 64). Bispecific antibodies were expressed by Expi293™ cells and purified by MabSelect™ SuRe affinity chromatography. Selected representatives are listed in Table 12 to illustrate the pairing algorithm and bispecific antibody sequence ID.

TABLE 12
Anti-cMET bispecific antibodies
pairing algorithm and sequence ID

| | |
|---|---|
| BsAb_seq ID | Arm2 |

| | | |
|---|---|---|
| and pairing | 063Ab03210 | 063Ab16720 |

| | | | |
|---|---|---|---|
| Arm1 | 063Ab10910 | 06BS01 | 06BS02 |
| | 061Ab15310 | 06BS03 | 06BS04 |
| | 063Ab16010 | 06BS05 | 06BS06 |
| | 063Ab02110 | 06BS07 | 06BS08 |
| | 063Ab15210 | 06BS09 | 06BS10 |
| | 062Ab16310 | 06BS11 | 06BS12 |
| | 063Ab05510 | 06BS13 | 06BS14 |
| | 063Ab07710 | 06BS15 | 06BS16 |
| | 063Ab14710 | 06BS17 | 06BS18 |
| | 061Ab05110 | 06BS19 | 06BS20 |

### Example 9. Inhibition of Ligand-Independent Signaling by Bispecific Anti-cMET Antibodies

For cancer cell lines with cMET amplification, like Hs746T (human gastric cancer cell), the cMET signaling is constitutively active to support cell proliferation without the need for ligand engagement. To assess the activity of anti-cMET bispecific antibodies to inhibit the ligand-independent cMET signaling, the inhibition of ERK phospho-T202/Y204 was measured in Hs746T cells upon anti-cMET bispecific antibodies treatment. Briefly, cell Hs746T was seeded in a 96-well assay plate and incubated at 37° C., 5% C02. After 24 hours, cells were treated with 10 nM of anti-cMET bispecific antibody and incubated at 37° C. for 18 hours. Then, the medium was discarded and 1×lysis buffer was added to the assay plate and incubated at room temperature for 30 minutes. Cellular phosphor-ERK was measured using ERK phospho-T202/Y204 Kit (PerkinElmer). The percentage of inhibition was calculated using the equation: % inhibition=100*[1−(X−MIN)/(MAX−MIN)]. X is the FRET signal at a given compound concentration. MAX is the signal in the presence of Hs746T and PBS only. MIN is the signal in the presence of medium only as the background signal. As shown in Table 13, all paired bispecific antibodies exhibited inhibitory activity on ligand-independent cMET signaling at 10 nM.

TABLE 13
Inhibition of ligand-independent signaling
by bispecific anti-cMET antibodies

Antibodies

| | | Bispecific | pERK inhibition % |
|---|---|---|---|
| Arm 1 | Arm2 | antibody | (at 10 nM) |
| 063Ab10910 | 063Ab03210 | 06BS01 | 75.54 |
| 063Ab10910 | 063Ab16720 | 06BS02 | 67.97 |
| 063Ab14710 | 063Ab03210 | 06BS17 | 68.94 |
| 063Ab14710 | 063Ab16720 | 06BS18 | 56.1 |
| 063Ab07710 | 063Ab03210 | 06BS15 | 73.88 |
| 063Ab07710 | 063Ab16720 | 06BS16 | 60.14 |
| 063Ab02110 | 063Ab03210 | 06BS07 | 75.01 |
| 063Ab02110 | 063Ab16720 | 06BS08 | 69.03 |
| 063Ab15210 | 063Ab03210 | 06BS09 | 73.86 |
| 063Ab15210 | 063Ab16720 | 06BS10 | 71.17 |
| 062Ab16310 | 063Ab03210 | 06BS11 | 74.51 |
| 062Ab16310 | 063Ab16720 | 06BS12 | 69.13 |
| 061Ab15310 | 063Ab03210 | 06BS03 | 72.8 |
| 061Ab15310 | 063Ab16720 | 06BS04 | 67.78 |
| 063Ab16010 | 063Ab03210 | 06BS05 | 72.36 |
| 063Ab16010 | 063Ab16720 | 06BS06 | 57.59 |
| 063Ab05510 | 063Ab03210 | 06BS13 | 70.16 |
| 063Ab05510 | 063Ab16720 | 06BS14 | 73.29 |
| 061Ab05110 | 063Ab03210 | 06BS19 | 67.18 |
| 061Ab05110 | 063Ab16720 | 06BS20 | 66.85 |
| hIgG | | | 2.35 |

### Example 10. Inhibitory Activity of Ligand-Independent Signaling and Anti-Proliferation Activity of Bispecific Anti-cMET Antibodies

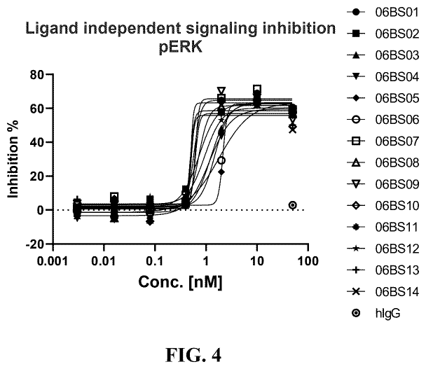

The ligand-independent signaling inhibitory activity of anti-cMET bispecific antibodies was measured by the method described in Example 9. Briefly, Hs746T cells were seeded to 96-well assay plate and treated with a series of dilutions of anti-cMET antibodies and incubated at 37° C. for 18 hours. Then, cellular phosphor-ERK was measured using ERK phospho-T202/Y204 Kit (PerkinElmer). IC50 values were determined by fitting the dose-response data to the four-parameter logistic model with GraphPad Prism. As shown in Table 14 and FIG. 4, all bispecific antibodies tested below 06BS01-06BS14 exhibited potent ligand-independent signaling inhibitory activity.

TABLE 14
Inhibition of ligand-independent signaling
by bispecific anti-cMET antibodies

| Antibodies | IC50 (nM) | Imax % |
|---|---|---|
| 06BS01 | 0.58 | 68.85 |
| 06BS02 | 0.73 | 67.57 |
| 06BS03 | 0.5 | 66.19 |
| 06BS04 | 0.49 | 63.94 |
| 06BS05 | 1.49 | 67.22 |
| 06BS06 | 0.94 | 63.53 |
| 06BS07 | 1.24 | 66.16 |
| 06BS08 | 1.15 | 63.07 |
| 06BS09 | 2.15 | 68.98 |
| 06BS10 | 1.82 | 64.89 |
| 06BS11 | 0.62 | 71.43 |
| 06BS12 | 0.72 | 66.7 |
| 06BS13 | 0.62 | 63.46 |
| 06BS14 | 0.51 | 61.93 |
| hIgG | N/A | 2.93 |

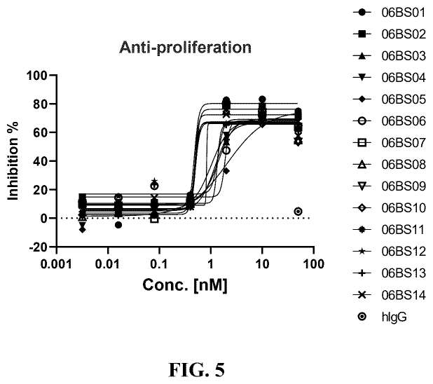

The anti-proliferation activity of anti-cMET bispecific antibodies was measured on cMET signaling dependent cell Hs746T. Cell-Titer-Glo assay (Promega) was used as a cell-based assay to evaluate the cell viability after anti-cMET antibodies treatment. Briefly, Hs746T cells were seeded to 96-well assay plate, cells were treated with a series of dilutions of anti-cMET bispecific antibodies and incubated at 37° C., 5% CO₂ for 6 days. After the antibodies treatment, 50 μl of Cell-Titer-Glo reagent was added in each well. Mixture was mixed on an orbital shaker for 10 min to allow cell lysis. Luminescent signal was measured using PheraStar (BMG Labtech) with luminescent protocol. IC50 values were determined by fitting the dose-response data to the four-parameter logistic model with GraphPad Prism. As shown in Table 15 and FIG. 5, all bispecific antibodies tested below 06BS01-06BS014 exhibited potent anti-proliferation activity on Hs746T, a cell line relies on constitutively activated cMET signaling.

TABLE 15
Anti-proliferation activity of bispecific
anti-cMET antibodies on Hs746T

| Antibodies | IC50(nM) | Imax % |
|---|---|---|
| 06BS01 | 0.48 | 83.24 |
| 06BS02 | 0.51 | 79.09 |
| 06BS03 | 0.51 | 77.17 |
| 06BS04 | 0.51 | 80.78 |
| 06BS05 | 1.2 | 70.8 |
| 06BS06 | 1.37 | 73.5 |
| 06BS07 | 1.39 | 75.39 |
| 06BS08 | 1.02 | 74.93 |
| 06BS09 | 2.08 | 71.29 |
| 06BS10 | 1.91 | 71.62 |
| 06BS11 | 0.49 | 79.26 |
| 06BS12 | 0.48 | 74.91 |
| 06BS13 | 1.34 | 70.28 |
| 06BS14 | 0.89 | 68.31 |
| hIgG | N/A | 4.75 |

### Example 11. Blocking Activity on Ligand-Induced Signaling by Bispecific Anti-cMET Antibodies

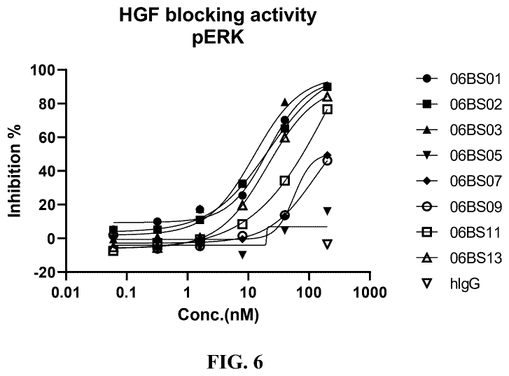

To assess the activity of anti-cMET bispecific antibodies to interfere with the HGF-cMET interaction and inhibit downstream signaling, the inhibition of HGF-induced ERK phospho-T202/Y204 upon antibody treatment was used as a readout. Briefly, H596 cells (epithelial-like cell line isolated from the lung cancer) were seeded to the 96-well assay plate and incubated at 37° C., 5% CO₂. After 24 hours, cells were starved with medium without FBS for 4 hours. Then, a series of dilutions of anti-cMET antibodies were added to each well and incubated at 37° C. for 1 hour. 100 pM human HGF was added to each well and incubated at 37° C. for 15 minutes. Then, the medium was discarded and 1×lysis buffer was added to the assay plate and incubated at room temperature for 30 minutes. Cellular phosphor-ERK was measured using ERK phospho-T202/Y204 Kit (PerkinElmer). IC50 values were determined by fitting the dose-response data to the four-parameter logistic model with GraphPad Prism. As shown in Table 16 and FIG. 6, bispecific anti-cMET antibodies 06BS01, 06BS02, 06BS03, 06BS11, 06BS13 exhibited strong activities of blocking HGF-induced cMET signaling.

TABLE 16
Blocking activity on ligand-induced signaling
of bispecific anti-cMET antibodies

| Antibodies | IC50 (nM) | Imax % |
|---|---|---|
| 06BS01 | 21.08 | 90.25 |
| 06BS02 | 21.7 | 89.99 |
| 06BS03 | 12.49 | 90.72 |
| 06BS05 | 40.23 | 16.04 |
| 06BS07 | 56.75 | 49.32 |
| 06BS09 | 50.75 | 50.75 |
| 06BS11 | 39.9 | 76.62 |
| 06BS13 | 20.67 | 84.18 |
| hIgG | N/A | −3.55 |

### Example 12. Removal of Post-Translational Modification Sites of Anti-cMET Antibodies

Point mutation was performed to remove some potential post-translational modification (PTM) sites of cMET antibodies.

For cMET antibodies 063Ab03210, 061Ab15310, and 063Ab10910, N terminal Glutamine (Q) of VH was mutated to Glutamic acid (E) to avoid the heterogeneity caused by cyclization of N terminal Q, resulting cMET antibodies 063Ab03210-V1 (see SEQ ID NOS: 82 and 64), 061Ab15310-V1 (SEQ ID NOS: 73 and 64), and 063Ab10910-V1 (see SEQ ID Nos: 72 and 64). As shown in Table 17, cMET antibodies 063Ab03210-V1 showed similar KD as parental antibody 063Ab03210, cMET antibodies 061Ab15310-V1 showed similar KD as parental antibody 061Ab15310, and cMET antibodies 063Ab10910-V1 showed similar KD as parental antibody 063Ab10910. The engineered VH sequences of cMET antibodies are shown in Table 18.

TABLE 17
SPR binding affinity of cMET antibodies 063Ab03210,
061Ab15310, 063Ab10910 and the variants thereof.

| Antibody variants | ka (1/Ms) | kd (1/s) | KD (M) |
|---|---|---|---|
| 063Ab03210 | 2.72E+5 | 2.01E−2 | 7.37E−8 |
| 063Ab03210-V1 | 2.69E+5 | 1.90E−2 | 7.05E−8 |
| 061Ab15310 | 9.73E+4 | 3.81E−3 | 3.92E−8 |
| 061Ab15310-V1 | 9.98E+4 | 3.61E−3 | 3.61E−8 |
| 063Ab10910 | 1.27E+5 | 7.14E−4 | 5.61E−9 |
| 063Ab10910-V1 | 1.26E+5 | 6.94E−4 | 5.49E−9 |

TABLE 18
Engineered VH sequences of cMET antibodies

| | SEQ ID | | |
|---|---|---|---|
| Antibody | NO | Description | SEQUENCE |
| 063Ab10910- | SEQ ID | VH (AA) | EVQLVESGGGVVQPGRSLRLSCAASGFTFSSHGMHWVRQA |
| V1 | NO: 72 | | PGKGLEWVAVISYDESDKYYADSVKGRFTISRDHSKNTLYL |
| | | | QMNSLRAEDTAVYYCVKDRNMGYSGWLDYWGQGTLVTV |
| | | | SS |
| 061Ab15310- | SEQ ID | VH (AA) | EVQLVESGGGVVQPGKSLRLSCAASGFTFSSSGLHWVRQA |
| V1 | NO: 73 | | PGKGLEWVAVISYDTSNKYYADSVKGRFTISRDNSKNTLYL |
| | | | QMNSLRAEDTAVYYCAKDLNRGYDWGFDYWGQGTLVTV |
| | | | SS |
| 063Ab16010- | SEQ ID | VH (AA) | EVQLVESGGGVVQPGRSLRLSCAASGFTFSNYGMHWVRQ |
| V1 | NO: 74 | | APGKGLEWVAVISYDGVDKYYADSVKGRFTISRDNSKNTL |
| | | | YLQMNSLRAEDTAMYYCAKDRISYNWNLDYWGQGTLVT |
| | | | VSS |
| 063Ab02110- | SEQ ID | VH (AA) | EVQLVESGGGVVQPGRSLRLSCVASGFTFSNYGLHWVRQA |
| V1 | NO: 75 | | PGKGLEWVAAISYDGSDKYYADPVKGRFTISRDTTKNTLSL |
| | | | QMNSLRAEDTAVYYCAKERYRGYDWDFDYWGQGTLVTV |
| | | | SS |
| 063Ab15210- | SEQ ID | VH (AA) | EVQLVESGGGVVQPGRSLRLSCAASGFTFSSYGLHWVRQA |
| V1 | NO: 76 | | PGKGLEWVAVISFDGSNKYYADSVKGRFTISRDTSKNTLSL |
| | | | QMNSLRAEDTAVYYCAKERSRGYDWDFDYWGQGILVTVS |
| | | | S |
| 062Ab16310- | SEQ ID | VH (AA) | EVRLVESGGGVVQPGRSLRLSCAASGFTFSNYGMHWVRQA |
| V1 | NO: 77 | | PGKGLEWVAVISYDGSDKYYPDSVKGRFTISRDNSRNTLYL |
| | | | QMNSLSTEDTAVYYCAKDQYRGYDGTFDYWGQGTLVTVS |
| | | | S |
| 063Ab05510- | SEQ ID | VH (AA) | EVQLVESGGGVVQPGRSLRLSCAASGFIFSSYNMHWVRQA |
| V1 | NO: 78 | | PGKGLEWVAVISYDRVNKYYADSVKGRFTISRDNSKNTLY |
| | | | LQMNSLRVEDTAVYYCAKERERYFDWLDYWGQGTLVTVS |
| | | | S |
| 063Ab07710- | SEQ ID | VH (AA) | EVQLVESGGGVVQPGRSLRLSCAASGFTFSSYGMHWVRQA |
| V1 | NO: 79 | | PGKGLEWVAVISNDGSYKYYVDSVTGRFTISRDNSKNTLYL |
| | | | QMNSLRAEDTAVYYCAKDRNMGYSGWFDYWGQGSLVTV |
| | | | SS |
| 063Ab14710- | SEQ ID | VH (AA) | EVQLVESGGGVVQPGRSLRLSCAASGFTFSSHGMHWVRQA |
| V1 | NO: 80 | | PGKGLEWVAVISNDGSYKYYADSVTGRFTISRDNSKNTLYL |
| | | | QMNSLRAEDTAVYYCAKDRNMGYSGWFDYWGQGSLVTV |
| | | | SS |
| 061Ab05110- | SEQ ID | VH (AA) | EVQLVESGGGVVQPGRSLRLSCAASGFTFSNYGMHWVRQ |
| V1 | NO: 81 | | APGKGLEWVAVISNDGSEKYYADSVKGRFTISRDSSKNTLY |
| | | | LQMNSLRAEDTAVYYCAKAPWGSGTTGRLDYWGQGTLVT |
| | | | VSS |
| 063Ab03210- | SEQ ID | VH (AA) | EFQLQESGPGLVKPSETLSLTCTVSGGSISTSSYYWGWIRQP |
| V1 | NO: 82 | | PGKGLEWIGTIYYSGTTYYNPSLKSRVTISVDTSKNQFSLKL |
| | | | KSVTAADTSIYYCARHWPKYNSWFDPWGQGILVTVSS |
| 063Ab16720- | SEQ ID | VH (AA) | EFQLQESGPGLVKPSETLSLTCTVSGGSISTSSYYWGWIRQP |
| V1 | NO: 83 | | PGKGLEWIGTIYFSGTTYYNPSLTSRVSMSVDTSKNQFSLKL |
| | | | NSVTAADTSIYYCARHWPKYNSWFDPWGQGFLVTVSS |

CDR Engineering of cMET Antibody 063Ab10910

For 063Ab10910, the potential deamidation and oxidation sites of NM motif in HCDR3 were mutated to various amino acid residues. Surface plasmon resonance (Biacore 8K, GE Life Sciences) was used to characterize the relative binding affinity to cMET ECD protein (Cat #MET-H5227, ACRO Biosystems) after PTM sites removal. The HCDR and VH sequences for each variant are summarized in Table 19. The LCDR and light chain sequences (SEQ ID NO: 64) remain the same common light chain as that of parental antibody. The variants 063Ab10910-P28, 063Ab10910-P27, 063Ab10910-P37, 063Ab10910-P26, and 063Ab10910-P19 showed comparable binding affinity as that of parental cMET antibody 063Ab10910, as shown in Table 20.

TABLE 19
HCDR and VH sequences of 063Ab10910 and the variants thereof.

| Antibody | SEQ ID NO | Description | SEQUENCE |
|---|---|---|---|
| 063Ab10910 | SEQ ID NO: 6 | HCDR1 (Kabat) | SHGMH |
| | SEQ ID NO: 7 | HCDR2 (Kabat) | VISYDESDKYYADSVKG |
| | SEQ ID NO: 8 | HCDR3 (Kabat) | DRNMGYSGWLDY |
| | SEQ ID NO: 4 | VH (AA) | QVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRN |
| | | | MGYSGWLDYWGQGTLVTVSS |
| 063Ab10910- | SEQ ID NO: 6 | HCDR1 (Kabat) | SHGMH |
| P28 | SEQ ID NO: 7 | HCDR2 (Kabat) | VISYDESDKYYADSVKG |
| | SEQ ID NO: 84 | HCDR3 (Kabat) | DRWEGYSGWLDY |
| | SEQ ID NO: 85 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDR |
| | | | WEGYSGWLDYWGQGTLVTVSS |
| 063Ab10910- | SEQ ID NO: 6 | HCDR1 (Kabat) | SHGMH |
| P27 | SEQ ID NO: 7 | HCDR2 (Kabat) | VISYDESDKYYADSVKG |
| | SEQ ID NO: 86 | HCDR3 (Kabat) | DRYEGYSGWLDY |
| | SEQ ID NO: 87 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRY |
| | | | EGYSGWLDYWGQGTLVTVSS |
| 063Ab10910- | SEQ ID NO: 6 | HCDR1 (Kabat) | SHGMH |
| P37 | SEQ ID NO: 7 | HCDR2 (Kabat) | VISYDESDKYYADSVKG |
| | SEQ ID NO: 88 | HCDR3 (Kabat) | DRHEGYSGWLDY |
| | SEQ ID NO: 89 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRH |
| | | | EGYSGWLDYWGQGTLVTVSS |
| 063Ab10910- | SEQ ID NO: 6 | HCDR1 (Kabat) | SHGMH |
| P26 | SEQ ID NO: 7 | HCDR2 (Kabat) | VISYDESDKYYADSVKG |
| | SEQ ID NO: 90 | HCDR3 (Kabat) | DRFEGYSGWLDY |
| | SEQ ID NO: 91 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRF |
| | | | EGYSGWLDYWGQGTLVTVSS |
| 063Ab10910- | SEQ ID NO: 6 | HCDR1 (Kabat) | SHGMH |
| P19 | SEQ ID NO: 7 | HCDR2 (Kabat) | VISYDESDKYYADSVKG |
| | SEQ ID NO: 92 | HCDR3 (Kabat) | DRNEGYSGWLDY |
| | SEQ ID NO: 93 | VH (AA) | EVQLVESGGGVVQPGRSLRLSCAASGFTFSSHGM |
| | | | HWVRQAPGKGLEWVAVISYDESDKYYADSVKGR |
| | | | FTISRDHSKNTLYLQMNSLRAEDTAVYYCVKDRN |
| | | | EGYSGWLDYWGQGTLVTVSS |
| 063Ab10910 | SEQ ID NO: 6 | HCDR1 (Kabat) | SHGMH |
| variants | SEQ ID NO: 7 | HCDR2 (Kabat) | VISYDESDKYYADSVKG |
| | SEQ ID NO: 94 | HCDR3 (Kabat) | DRX1X2GYSGWLDY, wherein X1 = W, Y, H, F |
| | | | or N; and X2 = E. |

TABLE 20
SPR binding affinity of cMET antibodies
063Ab10910 and the variants thereof.

| Antibody variants | ka (1/Ms) | kd (1/s) | KD (M) |
|---|---|---|---|
| 063Ab10910 | 7.10E+04 | 6.07E−04 | 8.55E−09 |
| 063Ab10910-P28 | 7.00E+04 | 3.96E−04 | 5.66E−09 |
| 063Ab10910-P27 | 7.41E+04 | 4.96E−04 | 6.69E−09 |
| 063Ab10910-P37 | 7.05E+04 | 5.52E−04 | 7.83E−09 |
| 063Ab10910-P26 | 6.90E+04 | 5.48E−04 | 7.94E−09 |
| 063Ab10910-P19 | 5.14E+04 | 1.06E−03 | 2.06E−08 |

### Example 13. Construction of Anti-EGFR×cMET×cMET Trispecific Antibody

Anti-EGFR×cMET×cMET trispecific antibodies, wherein two cMET arms binds to different anti-cMET epitopes, were constructed with VH and VL sequences from anti-cMET antibodies 063Ab10910-P19 and 063Ab03210-V1, and anti-EGFR antibody (SEQ ID No: 142 for VH and SEQ ID No: 143 for VL). The Fe region employed “knob into hole” technology to facilitate Fc heterodimerization to form bispecific cMET antibodies comprised of 063Ab10910-P19 and 063Ab03210-V1 arms. Anti-EGFR antibody was re-formatted to scFv and then fused to Fc C terminal of biparatopic anti-cMET antibody with GS linker sequence (GGGGS)₄ (SEQ ID NO: 139). The framework of two anti-cMET arms (063Ab10910-P19 and 063Ab03210-V1) were further engineered with multiple positive charge or negative charge amino acid to bring in pI difference between Fc heterodimer and homodimer thus to facilitate purification of target trispecific molecule in ion exchange chromatography, resulting in BGA-109 (VH: SEQ ID NO: 144) and BGA-032 (VH: SEQ ID NO: 145). The sequences of constructed trispecific antibody TE-642 were summarized in Table 21.

TABLE 21
Sequences for construction of anti-EGFR x cMET x cMET trispecific antibody
TE-642

| | SEQ ID NO | Description | SEQUENCE |
|---|---|---|---|
| EGFR arm | SEQ ID NO: 142 | VH sequence | QVQLVESGGGVVQPGRSLRLSCAASGFTFSTY |
| | | | GMHWVRQAPGKGLEWVAVIWDDGSYKYYG |
| | | | DSVKGRFTISRDNSKNTLYLQMNSLRAEDTAV |
| | | | YYCARDGITMVRGVMKDYFDYWGQGTLVTV |
| | | | SS |
| | SEQ ID NO: 155 | HCDR1 (Kabat) | TYGMH |
| | SEQ ID NO: 156 | HCDR2 (Kabat) | VIWDDGSYKYYGDSVKG |
| | SEQ ID NO: 157 | HCDR3 (Kabat) | DGITMVRGVMKDYFDY |
| | SEQ ID NO: 143 | VL sequence | AIQLTQSPSSLSASVGDRVTITCRASQDISSALV |
| | | | WYQQKPGKAPKLLIYDASSLESGVPSRFSGSES |
| | | | GTDFTLTISSLQPEDFATYYCQQFNSYPLTFGG |
| | | | GTKVEIK |
| | SEQ ID NO: 158 | LCDR1 (Kabat) | RASQDISSALV |
| | SEQ ID NO: 159 | LCDR2 (Kabat) | DASSLES |
| | SEQ ID NO: 160 | LCDR3 (Kabat) | QQFNSYPLT |
| EGFR arm | SEQ ID NO: 161 | ScFv (VL-VH) | AIQLTQSPSSLSASVGDRVTITCRASQDISSALV |
| | | | WYQQKPGKAPKLLIYDASSLESGVPSRFSGSES |
| | | | GTDFTLTISSLQPEDFATYYCQQFNSYPLTFGG |
| | | | GTKVEIKGGGGSGGGGSGGGGSGGGGSQVQL |
| | | | VESGGGVVQPGRSLRLSCAASGFTFSTYGMH |
| | | | WVRQAPGKGLEWVAVIWDDGSYKYYGDSVK |
| | | | GRFTISRDNSKNTLYLQMNSLRAEDTAVYYCA |
| | | | RDGITMVRGVMKDYFDYWGQGTLVTVSS |
| cMET arm 1 | SEQ ID NO: 144 | VH (AA) | EVQLVESGGEVVQPGESLRLSCAASGFTFSSHG |
| BGA-109 | | (063Ab10910-P19 + | MHWVRQAPGKGLEWVAVISYDESDKYYADS |
| | | charge engineering | VKGRFTISRDHSKNTLYLEMNSLRAEDTAVYY |
| | | in FR) | CVKDRNEGYSGWLDYWGQGTLVTVSS |
| | SEQ ID NO: 162 | VH (DNA) | GAAGTGCAGCTGGTGGAGTCCGGGGGCGAG |
| | | | GTGGTCCAACCCGGGGAGAGCCTGAGACTG |
| | | | AGCTGCGCCGCTAGCGGCTTCACCTTCAGCA |
| | | | GCCACGGCATGCACTGGGTGAGACAAGCCC |
| | | | CCGGCAAGGGCCTGGAGTGGGTGGCCGTGA |
| | | | TCAGCTACGACGAGAGCGACAAGTACTACG |
| | | | CCGACAGCGTGAAGGGCAGATTCACCATCA |
| | | | GCAGAGACCACAGCAAGAACACCCTGTACC |
| | | | TGGAGATGAACAGCCTGAGAGCCGAGGACA |
| | | | CCGCCGTGTACTACTGCGTGAAGGACAGAAA |
| | | | CGAGGGCTACAGCGGCTGGCTGGACTACTGG |
| | | | GGCCAAGGCACCCTGGTGACCGTGAGCAGC |
| | SEQ ID NO: 6 | HCDR1 (Kabat) | SHGMH |
| | SEQ ID NO: 7 | HCDR2 (Kabat) | VISYDESDKYYADSVKG |
| | SEQ ID NO: 92 | HCDR3 (Kabat) | DRNEGYSGWLDY |
| | SEQ ID NO: 64 | VL | EIVLTQSPATLSLSPGERATLSCRASQSVSSYLA |
| | | | WYQQKPGQAPRLLIYDASNRATGIPARFSGSG |
| | | | SGTDFTLTISSLEPEDFAVYYCQQRSNWPPTFG |
| | | | QGTKVEIK |
| | SEQ ID NO: 66 | LCDR1 (Kabat) | RASQSVSSYLA |
| | SEQ ID NO: 67 | LCDR2 (Kabat) | DASNRAT |
| | SEQ ID NO: 68 | LCDR3 (Kabat) | QQRSNWPPT |
| cMET arm 2 | SEQ ID NO: 145 | VH (AA) | EFQLQESGPGLVKPSETLRLTCTVSGGSISTSSY |
| BGA-032 | | (063Ab03210-V1 + | YWGWIRQPPGKGLEWIGTIYYSGTTYYNPSLK |
| | | charge engineering) | SRVTISVDTSKNQFSLKLKSVRAADTSIYYCAR |
| | | | HWPKYNSWFDPWGQGILVTVSS |
| | SEQ ID NO: 163 | VH (DNA) | GAGTTTCAGCTGCAAGAGAGCGGCCCCGGCC |
| | | | TGGTGAAGCCTAGCGAGACCCTGAGACTGAC |
| | | | CTGCACCGTGAGCGGCGGCAGCATCAGCAC |
| | | | AAGCAGCTACTACTGGGGCTGGATCAGACA |
| | | | GCCCCCCGGCAAGGGCCTGGAGTGGATCGG |
| | | | CACCATCTACTACAGCGGCACCACCTACTAC |
| | | | AACCCTAGCCTGAAGAGCAGAGTGACCATC |
| | | | AGCGTGGACACAAGCAAGAATCAGTTCAGC |
| | | | CTGAAGCTGAAGAGCGTGAGAGCCGCCGAC |
| | | | ACAAGCATCTACTACTGCGCTAGACACTGGC |
| | | | CCAAGTACAACAGCTGGTTCGACCCCTGGGG |
| | | | CCAAGGCATCCTGGTGACCGTGAGCAGC |
| | SEQ ID NO: 56 | HCDR1 (Kabat) | TSSYYWG |
| | SEQ ID NO: 57 | HCDR2 (Kabat) | TIYYSGTTYYNPSLKS |
| | SEQ ID NO: 58 | HCDR3 (Kabat) | HWPKYNSWFDP |
| | SEQ ID NO: 64 | VL | EIVLTQSPATLSLSPGERATLSCRASQSVSSYLA |
| | | | WYQQKPGQAPRLLIYDASNRATGIPARFSGSG |
| | | | SGTDFTLTISSLEPEDFAVYYCQQRSNWPPTFG |
| | | | QGTKVEIK |
| | SEQ ID NO: 66 | LCDR1 (Kabat) | RASQSVSSYLA |
| | SEQ ID NO: 67 | LCDR2 (Kabat) | DASNRAT |
| | SEQ ID NO: 68 | LCDR3 (Kabat) | QQRSNWPPT |
| TE-642 | SEQ ID NO: 146 | Polypeptide 1 (Knob | EFQLQESGPGLVKPSETLRLTCTVSGGSISTSSY |
| | | chain) with BGA- | YWGWIRQPPGKGLEWIGTIYYSGTTYYNPSLK |
| | | 032 and EGFR arm | SRVTISVDTSKNQFSLKLKSVRAADTSIYYCAR |
| | | | HWPKYNSWFDPWGQGILVTVSSASTKGPSVFP |
| | | | LAPSSKSTSGGTAALGCLVKDYFPEPVTVSWN |
| | | | SGALTSGVHTFPAVLQSSGLYSLSSVVTVPSSS |
| | | | LGTQTYICNVNHKPSNTKVDKKVEPKSCDKTH |
| | | | TCPPCPAPELLGGPSVFLFPPKPKDTLMISRTPE |
| | | | VTCVVVDVSHEDPEVKFNWYVDGVEVHNAK |
| | | | TKPREEQYNSTYRVVSVLTVLHQDWLNGKEY |
| | | | KCKVSNKALPAPIEKTISKAKGQPREPQVYTLP |
| | | | PSREEMTKNQVSLWCLVKGFYPSDIAVEWESN |
| | | | GQPENNYKTTPPVLDSDGSFFLYSKLTVDKSR |
| | | | WQQGNVFSCSVMHEALHNHYTQKSLSLSPGK |
| | | | GGGGSGGGGSGGGGSGGGGSAIQLTQSPSSLS |
| | | | ASVGDRVTITCRASQDISSALVWYQQKPGKAP |
| | | | KLLIYDASSLESGVPSRFSGSESGTDFTLTISSLQ |
| | | | PEDFATYYCQQFNSYPLTFGGGTKVEIKGGGG |
| | | | SGGGGSGGGGSGGGGSQVQLVESGGGVVQPG |
| | | | RSLRLSCAASGFTFSTYGMHWVRQAPGKGLE |
| | | | WVAVIWDDGSYKYYGDSVKGRFTISRDNSKN |
| | | | TLYLQMNSLRAEDTAVYYCARDGITMVRGVM |
| | | | KDYFDYWGQGTLVTVSS |
| | SEQ ID NO: 147 | Polypeptide 2 (Hole | EVQLVESGGEVVQPGESLRLSCAASGFTFSSHG |
| | | chain) with BGA- | MHWVRQAPGKGLEWVAVISYDESDKYYADS |
| | | 109 and EGFR arm | VKGRFTISRDHSKNTLYLEMNSLRAEDTAVYY |
| | | | CVKDRNEGYSGWLDYWGQGTLVTVSSASTKG |
| | | | PSVFPLAPSSKSTSGGTAALGCLVKDYFPEPVT |
| | | | VSWNSGALTSGVHTFPAVLQSSGLYSLSSVVT |
| | | | VPSSSLGTQTYICNVNHKPSNTKVDKKVEPKS |
| | | | CDKTHTCPPCPAPELLGGPSVFLFPPKPKDTLM |
| | | | ISRTPEVTCVVVDVSHEDPEVKFNWYVDGVEV |
| | | | HNAKTKPREEQYNSTYRVVSVLTVLHQDWLN |
| | | | GKEYKCKVSNKALPAPIEKTISKAKGQPREPQV |
| | | | YTLPPSREEMTKNQVSLSCAVKGFYPSDIAVE |
| | | | WESNGQPENNYKTTPPVLDSDGSFFLVSKLTV |
| | | | DKSRWQQGNVFSCSVMHEALHNHYTQKSLSL |
| | | | SPGKGGGGSGGGGSGGGGSGGGGSAIQLTQSP |
| | | | SSLSASVGDRVTITCRASQDISSALVWYQQKPG |
| | | | KAPKLLIYDASSLESGVPSRFSGSESGTDFTLTI |
| | | | SSLQPEDFATYYCQQFNSYPLTFGGGTKVEIKG |
| | | | GGGSGGGGSGGGGSGGGGSQVQLVESGGGVV |
| | | | QPGRSLRLSCAASGFTESTYGMHWVRQAPGK |
| | | | GLEWVAVIWDDGSYKYYGDSVKGRFTISRDN |
| | | | SKNTLYLQMNSLRAEDTAVYYCARDGITMVR |
| | | | GVMKDYFDYWGQGTLVTVSS |
| | SEQ ID NO: 148 | Polypeptide 3 | EIVLTQSPATLSLSPGERATLSCRASQSVSSYLA |
| | | (Common LC) | WYQQKPGQAPRLLIYDASNRATGIPARFSGSG |
| | | | SGTDFTLTISSLEPEDFAVYYCQQRSNWPPTFG |
| | | | QGTKVEIKRTVAAPSVFIFPPSDEQLKSGTASV |
| | | | VCLLNNFYPREAKVQWKVDNALQSGNSQESV |
| | | | TEQDSKDSTYSLSSTLTLSKADYEKHKVYACE |
| | | | VTHQGLSSPVTKSFNRGEC |

The antibody was constructed with in-house IgG1/CK eukaryotic expression vector, produced with ExpiCHO expression system (Thermofisher Scientific) with 30 mg/L (0.1 mM) fucosyltransferase inhibitor 2F-Peracetyl-Fucose (Sigma-Aldrich) for inhibition of fucosylation for effector function enhancement, and purified with MabSelect SuRe protein A chromatography resin (Cytiva) followed by HP—SP cation exchange chromatography (Cytiva). Obtained antibody with desired purity was then used for further characterization.

### Example 14. Construction of Anti-EGFR×cMET×cMET Trispecific Antibodies with Multiple Formats

Besides TE-642 with two anti-EGFR scFv fused to Fc C terminal, multiple trispecific antibody formats were constructed with different EGFR arm valency and different orientations of EGFR arm for further optimization of functionality of these trispecific antibodies. Specifically, single valent or bivalent EGFR scFv was fused to Fc C terminal, light chain C terminal, light chain N terminal, and heavy chain N terminal. These different combination of valency and orientations gave rise to trispecific antibodies TE-644, TE-645, TE-646, TE-647, and TE-648. The format of these five antibodies, as well as TE-642, were illustrated as FIG. 7. The sequences of these antibodies were summarized in Table 22.

TABLE 22
Sequences for construction of anti-EGFR x cMET x cMET trispecific
antibodies with different formats

| | SEQ ID | | |
|---|---|---|---|
| Antibody | NO | Description | SEQUENCE |
| TE-644 | SEQ ID | Polypeptide 1 | EFQLQESGPGLVKPSETLRLTCTVSGGSISTSSYYWGWIRQP |
| | NO: 149 | (Knob chain) | PGKGLEWIGTIYYSGTTYYNPSLKSRVTISVDTSKNQFSLKL |
| | | with BGA-032 | KSVRAADTSIYYCARHWPKYNSWFDPWGQGILVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGK |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | with BGA-109 | LEMNSLRAEDTAVYYCVKDRNEGYSGWLDYWGQGTLVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 3 | EIVLTQSPATLSLSPGERATLSCRASQSVSSYLAWYQQKPG |
| | NO: 151 | with common | QAPRLLIYDASNRATGIPARFSGSGSGTDFTLTISSLEPEDFA |
| | | LC and EGFR | VYYCQQRSNWPPTFGQGTKVEIKRTVAAPSVFIFPPSDEQL |
| | | arm | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGECGGGGSGGGGSGGGGSGGGGSAIQLTQSPSS |
| | | | LSASVGDRVTITCRASQDISSALVWYQQKPGKAPKLLIYDA |
| | | | SSLESGVPSRFSGSESGTDFTLTISSLQPEDFATYYCQQFNSY |
| | | | PLTFGGGTKVEIKGGGGSGGGGSGGGGSGGGGSQVQLVES |
| | | | GGGVVQPGRSLRLSCAASGFTFSTYGMHWVRQAPGKGLE |
| | | | WVAVIWDDGSYKYYGDSVKGRFTISRDNSKNTLYLQMNS |
| | | | LRAEDTAVYYCARDGITMVRGVMKDYFDYWGQGTLVTV |
| | | | SS |
| TE-645 | SEQ ID | Polypeptide 1 | EFQLQESGPGLVKPSETLRLTCTVSGGSISTSSYYWGWIRQP |
| | NO: 149 | (Knob chain) | PGKGLEWIGTIYYSGTTYYNPSLKSRVTISVDTSKNQFSLKL |
| | | with BGA-032 | KSVRAADTSIYYCARHWPKYNSWFDPWGQGILVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGK |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | with BGA-109 | LEMNSLRAEDTAVYYCVKDRNEGYSGWLDYWGQGTLVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 3 | AIQLTQSPSSLSASVGDRVTITCRASQDISSALVWYQQKPG |
| | NO: 152 | EGFR arm and | KAPKLLIYDASSLESGVPSRFSGSESGTDFTLTISSLQPEDFA |
| | | common LC | TYYCQQFNSYPLTFGGGTKVEIKGGGGSGGGGSGGGGSGG |
| | | | GGSQVQLVESGGGVVQPGRSLRLSCAASGFTFSTYGMHW |
| | | | VRQAPGKGLEWVAVIWDDGSYKYYGDSVKGRFTISRDNS |
| | | | KNTLYLQMNSLRAEDTAVYYCARDGITMVRGVMKDYFD |
| | | | YWGQGTLVTVSSGGGGSGGGGSGGGGSGGGGSEIVLTQSP |
| | | | ATLSLSPGERATLSCRASQSVSSYLAWYQQKPGQAPRLLIY |
| | | | DASNRATGIPARFSGSGSGTDFTLTISSLEPEDFAVYYCQQR |
| | | | SNWPPTFGQGTKVEIKRTVAAPSVFIFPPSDEQLKSGTASVV |
| | | | CLLNNFYPREAKVQWKVDNALQSGNSQESVTEQDSKDST |
| | | | YSLSSTLTLSKADYEKHKVYACEVTHQGLSSPVTKSFNRGE |
| | | | C |
| TE-646 | SEQ ID | Polypeptide 1 | AIQLTQSPSSLSASVGDRVTITCRASQDISSALVWYQQKPG |
| | NO: 153 | (Knob chain) | KAPKLLIYDASSLESGVPSRFSGSESGTDFTLTISSLQPEDFA |
| | | with EGFR | TYYCQQFNSYPLTFGGGTKVEIKGGGGSGGGGSGGGGSGG |
| | | arm and BGA- | GGSQVQLVESGGGVVQPGRSLRLSCAASGFTFSTYGMHW |
| | | 032 | VRQAPGKGLEWVAVIWDDGSYKYYGDSVKGRFTISRDNS |
| | | | KNTLYLQMNSLRAEDTAVYYCARDGITMVRGVMKDYFD |
| | | | YWGQGTLVTVSSGGGGSGGGGSGGGGSGGGGSEFQLQES |
| | | | GPGLVKPSETLRLTCTVSGGSISTSSYYWGWIRQPPGKGLE |
| | | | WIGTIYYSGTTYYNPSLKSRVTISVDTSKNQFSLKLKSVRA |
| | | | ADTSIYYCARHWPKYNSWFDPWGQGILVTVSSASTKGPSV |
| | | | FPLAPSSKSTSGGTAALGCLVKDYFPEPVTVSWNSGALTSG |
| | | | VHTFPAVLQSSGLYSLSSVVTVPSSSLGTQTYICNVNHKPS |
| | | | NTKVDKKVEPKSCDKTHTCPPCPAPELLGGPSVFLFPPKPK |
| | | | DTLMISRTPEVTCVVVDVSHEDPEVKFNWYVDGVEVHNA |
| | | | KTKPREEQYNSTYRVVSVLTVLHQDWLNGKEYKCKVSNK |
| | | | ALPAPIEKTISKAKGQPREPQVYTLPPSREEMTKNQVSLWC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| | SEQ ID | Polypeptide 2 | AIQLTQSPSSLSASVGDRVTITCRASQDISSALVWYQQKPG |
| | NO: 154 | (Hole chain) | KAPKLLIYDASSLESGVPSRFSGSESGTDFTLTISSLQPEDFA |
| | | with EGFR | TYYCQQFNSYPLTFGGGTKVEIKGGGGSGGGGSGGGGSGG |
| | | arm and BGA- | GGSQVQLVESGGGVVQPGRSLRLSCAASGFTFSTYGMHW |
| | | 109 | VRQAPGKGLEWVAVIWDDGSYKYYGDSVKGRFTISRDNS |
| | | | KNTLYLQMNSLRAEDTAVYYCARDGITMVRGVMKDYFD |
| | | | YWGQGTLVTVSSGGGGSGGGGSGGGGSGGGGSEVQLVES |
| | | | GGEVVQPGESLRLSCAASGFTFSSHGMHWVRQAPGKGLE |
| | | | WVAVISYDESDKYYADSVKGRFTISRDHSKNTLYLEMNSL |
| | | | RAEDTAVYYCVKDRNEGYSGWLDYWGQGTLVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKFNWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LSCAVKGFYPSDIAVEWESNGQPENNYKTTPPVLDSDGSFF |
| | | | LVSKLTVDKSRWQQGNVFSCSVMHEALHNHYTQKSLSLSP |
| | | | GK |
| | SEQ ID | Polypeptide 3 | EIVLTQSPATLSLSPGERATLSCRASQSVSSYLAWYQQKPG |
| | NO: 148 | (Common LC) | QAPRLLIYDASNRATGIPARFSGSGSGTDFTLTISSLEPEDFA |
| | | | VYYCQQRSNWPPTFGQGTKVEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |
| TE-647 | SEQ ID | Polypeptide 1 | AIQLTQSPSSLSASVGDRVTITCRASQDISSALVWYQQKPG |
| | NO: 153 | (Knob chain) | KAPKLLIYDASSLESGVPSRESGSESGTDFTLTISSLQPEDFA |
| | SEQ ID | with EGFR | TYYCQQFNSYPLTFGGGTKVEIKGGGGSGGGGSGGGGSGG |
| | NO: 164 | arm and BGA- | GGSQVQLVESGGGVVQPGRSLRLSCAASGFTFSTYGMHW |
| | | 032 | VRQAPGKGLEWVAVIWDDGSYKYYGDSVKGRFTISRDNS |
| | | AA | KNTLYLQMNSLRAEDTAVYYCARDGITMVRGVMKDYFD |
| | | | YWGQGTLVTVSSGGGGSGGGGSGGGGSGGGGSEFQLQES |
| | | | GPGLVKPSETLRLTCTVSGGSISTSSYYWGWIRQPPGKGLE |
| | | | WIGTIYYSGTTYYNPSLKSRVTISVDTSKNQFSLKLKSVRA |
| | | | ADTSIYYCARHWPKYNSWFDPWGQGILVTVSSASTKGPSV |
| | | | FPLAPSSKSTSGGTAALGCLVKDYFPEPVTVSWNSGALTSG |
| | | | VHTFPAVLQSSGLYSLSSVVTVPSSSLGTQTYICNVNHKPS |
| | | | NTKVDKKVEPKSCDKTHTCPPCPAPELLGGPSVFLFPPKPK |
| | | | DTLMISRTPEVTCVVVDVSHEDPEVKFNWYVDGVEVHNA |
| | | | KTKPREEQYNSTYRVVSVLTVLHQDWLNGKEYKCKVSNK |
| | | | ALPAPIEKTISKAKGQPREPQVYTLPPSREEMTKNQVSLWC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| | | Polypeptide 1 | GCCATTCAGCTGACACAGAGCCCTAGCAGCCTGAGCGCT |
| | | (Knob chain) | AGCGTGGGCGACAGAGTGACAATCACCTGCAGAGCTAG |
| | | with EGFR | CCAAGACATCAGCAGCGCCCTGGTGTGGTATCAGCAGA |
| | | arm and BGA- | AGCCCGGCAAGGCCCCCAAGCTGCTGATCTACGACGCTA |
| | | 032 | GCAGCCTCGAAAGCGGCGTGCCTAGCAGATTTAGCGGC |
| | | DNA | AGCGAGAGCGGCACCGACTTCACCCTGACCATCAGCAG |
| | | | CCTGCAGCCCGAGGACTTCGCCACCTACTACTGTCAGCA |
| | | | GTTCAACAGCTACCCCCTGACCTTCGGCGGGGGCACAAA |
| | | | AGTGGAAATTAAGGGTGGAGGCGGTTCAGGCGGAGGTG |
| | | | GCTCTGGCGGTGGCGGATCGGGTGGCGGAGGCTCACAA |
| | | | GTGCAACTGGTCGAAAGCGGCGGGGGGGTGGTGCAGCC |
| | | | TGGGAGAAGCCTGAGACTGAGCTGCGCCGCTAGCGGCT |
| | | | TCACCTTCAGCACCTACGGCATGCACTGGGTGAGACAAG |
| | | | CCCCCGGCAAAGGCCTGGAATGGGTGGCCGTGATCTGG |
| | | | GACGACGGCAGCTACAAGTACTACGGCGACAGCGTGAA |
| | | | GGGCAGATTCACCATCAGCAGAGACAACAGCAAGAACA |
| | | | CCCTGTACCTGCAGATGAACAGCCTGAGAGCCGAGGAC |
| | | | ACCGCCGTGTACTACTGCGCTAGAGACGGCATCACCATG |
| | | | GTGAGAGGCGTGATGAAGGACTACTTTGACTATTGGGG |
| | | | GCAAGGCACCCTGGTGACCGTGAGCAGCGGTGGAGGCG |
| | | | GTTCAGGCGGAGGTGGCTCTGGCGGTGGCGGATCGGGT |
| | | | GGCGGAGGCTCAGAGTTTCAGCTGCAAGAGTCCGGCCC |
| | | | CGGCCTGGTGAAGCCTAGCGAGACCCTGAGACTGACCT |
| | | | GCACCGTGAGCGGCGGCAGCATCAGCACAAGCAGCTAC |
| | | | TACTGGGGCTGGATCAGACAGCCTCCCGGCAAGGGCCT |
| | | | GGAGTGGATCGGCACCATCTACTACAGCGGCACCACCTA |
| | | | CTACAACCCTAGCCTGAAGAGCAGAGTGACCATCAGCG |
| | | | TGGACACAAGCAAGAATCAGTTCAGCCTGAAGCTGAAG |
| | | | AGCGTGAGAGCCGCCGACACAAGCATCTATTACTGCGCC |
| | | | CGGCACTGGCCCAAGTACAACAGCTGGTTCGACCCCTGG |
| | | | GGCCAAGGCATCCTGGTGACCGTCAGCAGCGCCTCCACA |
| | | | AAGGGGCCTAGCGTGTTCCCTCTGGCCCCTAGCAGCAAA |
| | | | AGCACAAGCGGCGGCACCGCTGCCCTGGGCTGCCTGGTC |
| | | | AAGGATTACTTCCCCGAGCCCGTGACCGTGAGCTGGAAT |
| | | | AGCGGCGCTCTCACAAGCGGCGTGCACACCTTCCCCGCC |
| | | | GTGCTGCAGAGCAGCGGCCTGTACAGCCTGAGCAGCGT |
| | | | GGTGACCGTGCCTAGCAGCAGCCTGGGCACACAGACCT |
| | | | ACATCTGCAACGTGAACCACAAGCCTAGCAACACCAAG |
| | | | GTGGACAAAAAGGTGGAGCCCAAGTCCTGTGACAAGAC |
| | | | CCACACCTGCCCCCCCTGTCCCGCCCCCGAACTCCTGGG |
| | | | CGGCCCTAGCGTGTTCCTGTTCCCCCCCAAGCCCAAGGA |
| | | | CACCCTGATGATCAGCAGAACCCCCGAGGTGACCTGCGT |
| | | | GGTCGTGGACGTGAGCCACGAGGACCCCGAGGTGAAGT |
| | | | TCAACTGGTACGTGGACGGCGTGGAGGTGCACAACGCC |
| | | | AAGACCAAGCCTAGAGAGGAGCAGTACAACAGCACCTA |
| | | | CAGAGTGGTGAGCGTGCTGACCGTGCTGCACCAAGACT |
| | | | GGCTGAACGGCAAGGAGTACAAGTGCAAGGTGAGCAAC |
| | | | AAGGCCCTGCCCGCCCCCATCGAGAAGACCATCAGCAA |
| | | | GGCCAAGGGGCAGCCTAGAGAGCCCCAAGTGTACACCC |
| | | | TGCCCCCTAGCAGAGAGGAGATGACCAAGAACCAAGTG |
| | | | AGCCTGTGGTGCCTGGTGAAGGGGTTTTACCCTAGCGAC |
| | | | ATCGCCGTGGAGTGGGAGAGCAACGGGCAGCCCGAGAA |
| | | | CAACTACAAGACCACCCCCCCCGTGCTGGACAGCGACG |
| | | | GCAGCTTCTTCCTGTACAGCAAGCTGACCGTGGACAAGA |
| | | | GCAGATGGCAGCAAGGCAACGTGTTCAGCTGCAGCGTG |
| | | | ATGCACGAGGCCCTGCACAACCACTACACACAGAAGAG |
| | | | CCTGAGCCTGAGCCCCGGCAAG |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | SEQ ID | with BGA-109 | LEMNSLRAEDTAVYYCVKDRNEGYSGWLDYWGQGTLVT |
| | NO: 165 | AA | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | | Polypeptide 2 | GAGGTGCAGCTGGTCGAGAGCGGGGGCGAAGTGGTCCA |
| | | (Hole chain) | ACCCGGCGAGAGCCTGAGACTGTCCTGCGCCGCTAGCG |
| | | with BGA-109 | GCTTCACCTTCAGCAGCCACGGCATGCACTGGGTGAGAC |
| | | DNA | AAGCCCCCGGCAAGGGCCTGGAGTGGGTGGCCGTGATC |
| | | | AGCTACGACGAGAGCGACAAGTACTACGCCGACAGCGT |
| | | | GAAGGGCAGATTCACCATCAGCAGAGACCACAGCAAGA |
| | | | ACACCCTGTACCTGGAGATGAACAGCCTGAGAGCCGAG |
| | | | GACACCGCCGTGTACTACTGCGTGAAGGACAGAAACGA |
| | | | GGGCTACAGCGGCTGGCTGGACTACTGGGGCCAAGGCA |
| | | | CCCTGGTGACCGTGTCCTCCGCTAGCACCAAGGGCCCTA |
| | | | GCGTGTTTCCCCTGGCCCCTAGCAGCAAGTCCACCTCCG |
| | | | GCGGGACAGCCGCTCTGGGCTGCCTGGTGAAGGACTACT |
| | | | TCCCCGAGCCCGTGACAGTGAGCTGGAACTCCGGCGCTC |
| | | | TGACAAGCGGCGTGCATACCTTCCCCGCCGTGCTGCAGA |
| | | | GCAGCGGCCTGTACAGCCTGAGCAGCGTGGTGACCGTG |
| | | | CCTAGCAGCAGCCTGGGCACACAGACCTACATCTGCAAC |
| | | | GTGAACCACAAGCCTAGCAACACCAAGGTGGACAAGAA |
| | | | GGTGGAGCCCAAATCCTGCGATAAAACCCACACATGCC |
| | | | CCCCCTGCCCCGCCCCCGAGCTGCTGGGCGGCCCCTCCG |
| | | | TCTTCCTGTTCCCCCCCAAGCCCAAGGACACCCTGATGA |
| | | | TCAGCAGAACCCCCGAGGTGACCTGCGTGGTCGTGGAC |
| | | | GTGAGCCACGAGGACCCCGAGGTGAAGTTCAACTGGTA |
| | | | CGTGGACGGCGTGGAGGTGCACAACGCCAAGACCAAGC |
| | | | CTAGAGAGGAGCAGTACAACAGCACCTACAGAGTGGTG |
| | | | AGCGTGCTGACCGTGCTGCACCAAGACTGGCTGAACGG |
| | | | CAAGGAGTACAAGTGCAAGGTGAGCAACAAGGCCCTGC |
| | | | CCGCCCCCATCGAGAAGACCATCAGCAAGGCCAAGGGG |
| | | | CAGCCTAGAGAGCCCCAAGTGTACACCCTGCCCCCTAGC |
| | | | AGAGAGGAGATGACCAAGAACCAAGTGAGCCTGAGCTG |
| | | | CGCCGTGAAGGGCTTCTACCCTAGCGACATCGCCGTGGA |
| | | | GTGGGAGAGCAACGGGCAGCCCGAGAACAACTACAAGA |
| | | | CCACCCCCCCCGTGCTGGACAGCGACGGCAGCTTCTTCC |
| | | | TGGTGAGCAAGCTGACCGTGGACAAGAGCAGATGGCAG |
| | | | CAAGGCAACGTGTTCAGCTGCAGCGTGATGCACGAGGC |
| | | | CCTGCACAACCACTACACACAGAAGAGCCTGAGCCTGA |
| | | | GCCCCGGCAAG |
| | SEQ ID | Polypeptide 3 | EIVLTQSPATLSLSPGERATLSCRASQSVSSYLAWYQQKPG |
| | NO: 148 | (Common LC) | QAPRLLIYDASNRATGIPARFSGSGSGTDFTLTISSLEPEDFA |
| | | AA | VYYCQQRSNWPPTFGQGTKVEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |
| | SEQ ID | Polypeptide 3 | GAGATTGTGCTGACACAGAGCCCCGCCACCCTGTCCCTG |
| | NO: 166 | (Common LC) | TCCCCCGGGGAGAGAGCCACCCTGAGCTGCAGAGCTAG |
| | | DNA | CCAAAGCGTGAGCAGCTACCTGGCCTGGTATCAGCAGA |
| | | | AGCCCGGCCAAGCCCCTAGACTGCTGATCTACGACGCTA |
| | | | GCAACAGAGCCACCGGCATCCCCGCTAGATTCAGCGGC |
| | | | AGCGGCAGCGGCACCGACTTCACCCTGACCATCAGCAG |
| | | | CCTGGAGCCCGAGGACTTCGCCGTGTACTACTGTCAGCA |
| | | | GAGAAGCAACTGGCCCCCCACCTTCGGCCAAGGCACCA |
| | | | AGGTGGAGATCAAGCGTACGGTGGCTGCACCATCTGTCT |
| | | | TCATCTTCCCGCCATCTGATGAGCAGTTGAAATCTGGAA |
| | | | CTGCCTCTGTTGTGTGCCTGCTGAATAACTTCTATCCCAG |
| | | | AGAGGCCAAAGTACAGTGGAAGGTGGATAACGCCCTCC |
| | | | AATCGGGTAACTCCCAGGAGAGTGTCACAGAGCAGGAC |
| | | | AGCAAGGACAGCACCTACAGCCTCAGCAGCACCCTGAC |
| | | | GCTGAGCAAAGCAGACTACGAGAAACACAAAGTCTACG |
| | | | CCTGCGAAGTCACCCATCAGGGCCTGAGCTCGCCCGTCA |
| | | | CAAAGAGCTTCAACAGGGGAGAGTGT |
| TE-648 | SEQ ID | Polypeptide 1 | EFQLQESGPGLVKPSETLRLTCTVSGGSISTSSYYWGWIRQP |
| | NO: 146 | (Knob chain) | PGKGLEWIGTIYYSGTTYYNPSLKSRVTISVDTSKNQFSLKL |
| | | | KSVRAADTSIYYCARHWPKYNSWFDPWGQGILVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKVEPKSCDKTHTCPPCPAPELLGGPSVFLFP |
| | | | PKPKDTLMISRTPEVTCVVVDVSHEDPEVKENWYVDGVEV |
| | | | HNAKTKPREEQYNSTYRVVSVLTVLHQDWLNGKEYKCKV |
| | | | SNKALPAPIEKTISKAKGQPREPQVYTLPPSREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGKGGGGSGGGGSGGGGSGGGGSAIQLTQSPSSLSASVGD |
| | | | RVTITCRASQDISSALVWYQQKPGKAPKLLIYDASSLESGV |
| | | | PSRFSGSESGTDFTLTISSLQPEDFATYYCQQFNSYPLTFGG |
| | | | GTKVEIKGGGGSGGGGSGGGGSGGGGSQVQLVESGGGVV |
| | | | QPGRSLRLSCAASGFTFSTYGMHWVRQAPGKGLEWVAVI |
| | | | WDDGSYKYYGDSVKGRFTISRDNSKNTLYLQMNSLRAED |
| | | | TAVYYCARDGITMVRGVMKDYFDYWGQGTLVTVSS |
| | SEQ ID | Polypeptide 2 | EVQLVESGGEVVQPGESLRLSCAASGFTFSSHGMHWVRQA |
| | NO: 150 | (Hole chain) | PGKGLEWVAVISYDESDKYYADSVKGRFTISRDHSKNTLY |
| | | | LEMNSLRAEDTAVYYCVKDRNEGYSGWLDYWGQGTLVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPAPIEKTISKAKGQPREPQVYTLPPSREEM |
| | | | TKNQVSLSCAVKGFYPSDIAVEWESNGQPENNYKTTPPVL |
| | | | DSDGSFFLVSKLTVDKSRWQQGNVFSCSVMHEALHNHYT |
| | | | QKSLSLSPGK |
| | SEQ ID | Polypeptide 3 | EIVLTQSPATLSLSPGERATLSCRASQSVSSYLAWYQQKPG |
| | NO: 148 | (Common LC) | QAPRLLIYDASNRATGIPARFSGSGSGTDFTLTISSLEPEDFA |
| | | | VYYCQQRSNWPPTFGQGTKVEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |

These five antibodies were constructed with in house IgG1/CK eukaryotic expression vector, produced with ExpiCHO expression system (Thermofisher Scientific) with 30 mg/l (0.1 mM) fucosyltransferase inhibitor 2F-Peracetyl-Fucose (Sigma-Aldrich) for inhibition of fucosylation for effector function enhancement, and purified with MabSelect SuRe protein A chromatography resin (Cytiva) followed by HP—SP cation exchange chromatography (Cytiva). Obtained antibodies with desired purity was then used for further characterization.

### Example 15. Binding Affinity of TE-647 to Human EGFR and cMET Proteins

Surface plasmon resonance (Biacore 8K, GE Life Sciences) was used to characterize the binding affinity of trispecific antibody TE-647, as well as control antibodies JNJ-372 (Amivantamab, sequences downloaded from https://www.imgt.org/mAb-DB/, internally generated with “controlled Fab-arm exchange” procedure¹⁰ and with 0.1 mM fucosyltransferase inhibitor 2F-Peracetyl-Fucose to inhibit fucosylation for effector function enhancement) and LY3164530 (Sequences extracted from patent WO2015100104A1), to human cMET ECD protein (Cat #10692-H08H, Sino Biological) and human EGFR protein (Cat #10001-H08H, Sino Biological). Briefly, anti-human IgG Fc antibody (Cat #29234600, GE Life Sciences) was immobilized via amine coupling onto an activated CM5 biosensor chip (Cat #29149603, GE Life Sciences). Trispecific antibody TE-647, or control antibodies JNJ-372 and LY3164530 were flowed over the chip surface and captured by anti-human IgG antibody. Then a serial dilution of human cMET and human EGFR proteins were injected over the antibodies captured surface and changes in surface plasmon resonance signals were analyzed to calculate the association rates (kₐ) and dissociation rates (k_{d}) by using the one-to-one Langmuir binding model (Biacore™ Insight Evaluation Software, GE Life Sciences). The equilibrium dissociation constant (KD) was calculated as the ratio kₐ/k_{d}. The binding affinity is summarized as in Table 23.

TABLE 23
SPR binding affinity of trispecific antibody TE-647 and
control antibodies to human cMET and human EGFR proteins.

| Test Ab | Test Antigen | ka (1/Ms) | kd (1/s) | KD (M) |
|---|---|---|---|---|
| TE-647 | Human cMet | 1.67E+05 | 2.49E−04 | 1.49E−09 |
| JNJ-372 | Human cMet | 1.85E+05 | 1.56E−04 | 8.45E−10 |
| LY3164530 | Human cMet | 7.84E+04 | 2.32E−04 | 2.96E−09 |
| TE-647 | Human EGFR | 1.50E+05 | 1.06E−03 | 7.08E−09 |
| JNJ-372 | Human EGFR | 9.49E+04 | 8.72E−04 | 9.19E−09 |
| LY3164530 | Human EGFR | 7.97E+05 | 8.65E−04 | 1.09E−09 |

### Example 16. ADCC Activity of EGFR×cMET×cMET Trispecific Antibodies

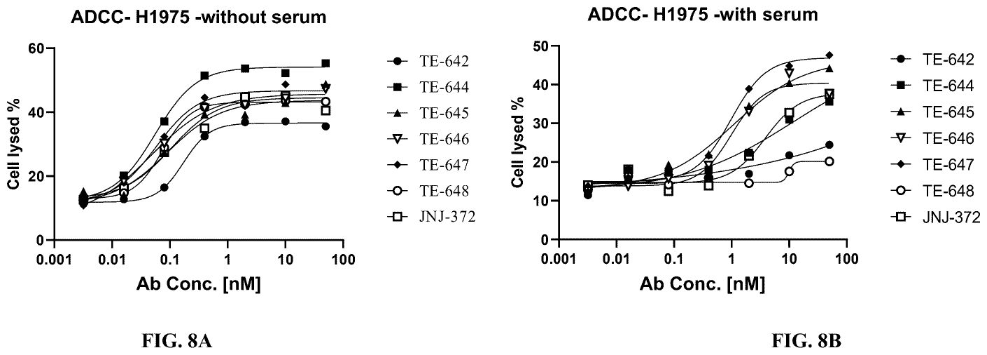

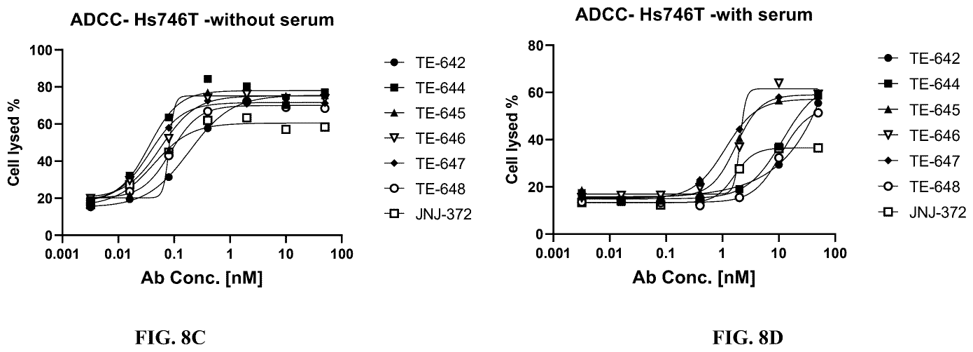

ADCC is one of the mechanisms contributing the anti-tumor activity of the constructed trispecific antibodies. The ADCC activity was evaluated against EGFR signaling-dependent cell line H1975 (cell line from non-small cell lung cancer, which is EGFR signaling dependent and ligand independent) and c-MET signaling-dependent cell line Hs746T (human gastric cancer cell, which is cMET signaling dependent and ligand independent) by co-culturing with human primary PBMC. H1975 and Hs746T cells were engineered to express nano-luciferase which was trapped in cytoplasm and will be released when cells are lysed. Briefly, human PBMC (1×10⁵) were co-cultured with H1975/Nluc (5×10³ per well) or Hs746T/Nluc (5×10³ per well) in the presence of test antibodies with series dilution (0.0032-50 nM) for 16 hours in 96-well V-bottom plates. Then, cell supernatants were collected and the luciferase signals were measured by Nano-Glo Luciferase Assay System (Promega, #N1120). The activities were measured in the presence or absence of whole human serum in cell culture medium. IC₅₀ values were determined by fitting the dose-response data to the four-parameter logistic model with GraphPad Prism. As shown in Table 24 and FIG. 8, the TE-644, TE-645, TE-646 and TE-647 exhibited better ADCC activity than JNJ-372 on H1975 (FIGS. 8A and 8B); and all below trispecific antibodies showed better activity than JNJ-372 on Hs746T (FIGS. 8C and 8D), either with serum or without serum.

TABLE 24
ADCC activity of trispecific antibodies

| | H1975 | | | | Hs746T | | | |
|---|---|---|---|---|---|---|---|---|
| | without serum | | with serum | | without serum | | with serum | |
| Antibody | IC₅₀ (nM) | Iₘₐₓ (%) | IC₅₀ (nM) | Iₘₐₓ (%) | IC₅₀ (nM) | Iₘₐₓ (%) | IC₅₀ (nM) | Iₘₐₓ (%) |
| TE-642 | 0.17 | 37.14 | 6.09 | 24.43 | 0.19 | 76.04 | 12.54 | 55.49 |
| TE-644 | 0.06 | 55.31 | 2.84 | 35.62 | 0.04 | 77.23 | 11.62 | 58.69 |
| TE-645 | 0.1 | 48.79 | 1.1 | 44.23 | 0.08 | 75.55 | 1.74 | 59.95 |
| TE-646 | 0.05 | 47.31 | 1 | 42.91 | 0.06 | 73.7 | 2.05 | 63.98 |
| TE-647 | 0.06 | 48.69 | 0.97 | 47.59 | 0.04 | 71.21 | 1.13 | 57.99 |
| TE-648 | 0.07 | 43.57 | 9.88 | 20.14 | 0.09 | 68.45 | 10.78 | 51.4 |
| JNJ-372 | 0.09 | 45.12 | 3.84 | 37.51 | 0.04 | 63.41 | 1.65 | 36.49 |

### Example 17. ADCP Activity of EGFR×cMET×cMET Trispecific Antibodies

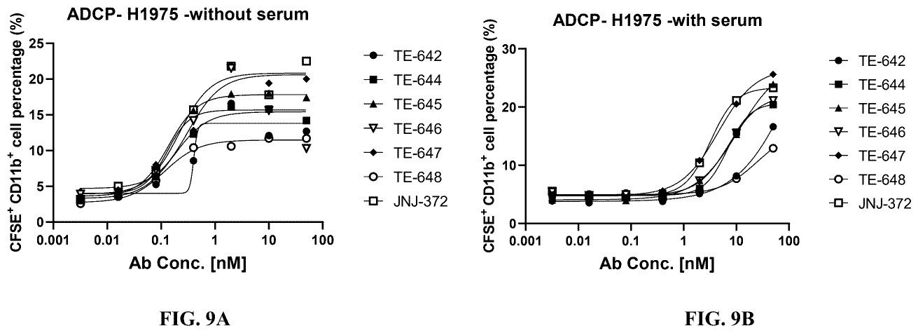

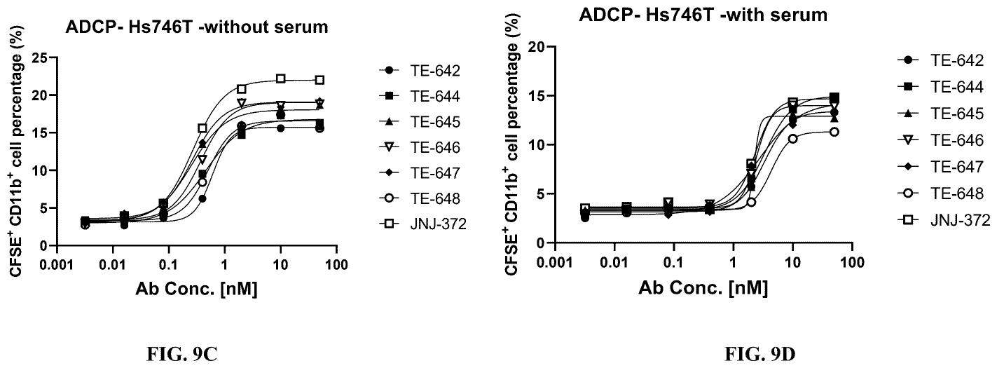

ADCP is another Fc-mediated function contributing to the anti-tumor activity of the constructed trispecific antibodies. The ADCP activity on H1975 and Hs756T was measured by using human macrophages. Briefly, human macrophage was derived and differentiated from human PBMC. The human macrophages (1×10⁵ per well) were co-cultured with CFSE pre-labeled H1975 (5×10⁴) or Hs746T (5×10⁴) in the presence of antibodies (0.0032-50 nM) for 2 hours in 96-well U-bottom plates, followed by flow cytometry to measure the percentage of CFSE-labeled and CD11b positive macrophages, which reflect the ADCP activity. The activities were measured in the presence or absence of whole human serum in cell culture medium. EC₅₀ values were determined by fitting the dose-response data to the four-parameter logistic model with GraphPad Prism. As shown in Table 25 and FIG. 9, all trispecific antibodies exhibited ADCP activity against tumor cells. TE-646 and TE-647 exhibited comparable ADCP activity as JNJ-372 on both H1975 (FIGS. 9A and 9B) and Hs746T (FIGS. 9C and 9D).

TABLE 25
ADCP activity of trispecific antibodies

| | H1975 | | | | Hs746t | | | |
|---|---|---|---|---|---|---|---|---|
| | without serum | | with serum | | without serum | | with serum | |
| Antibody | EC₅₀ (nM) | Eₘₐₓ (%) | EC₅₀ (nM) | Eₘₐₓ (%) | EC₅₀ (nM) | Eₘₐₓ (%) | EC₅₀ (nM) | Eₘₐₓ (%) |
| TE-642 | 0.4 | 16.6 | 12.81 | 16.6 | 0.6 | 15.8 | 3.35 | 13.3 |
| TE-644 | 0.17 | 16.1 | 6.78 | 20.4 | 0.45 | 17.3 | 3.35 | 14.9 |
| TE-645 | 0.14 | 18 | 7.7 | 23.9 | 0.25 | 18.7 | 2.26 | 13.1 |
| TE-646 | 0.13 | 21.5 | 7.1 | 21.1 | 0.37 | 18.8 | 2.44 | 14 |
| TE-647 | 0.32 | 21.9 | 4.55 | 25.6 | 0.26 | 19.2 | 2.75 | 14.3 |
| TE-648 | 0.12 | 11.7 | 12.47 | 12.9 | 0.5 | 17.4 | 4.32 | 11.3 |
| JNJ-372 | 0.21 | 22.5 | 3.21 | 23.3 | 0.27 | 22 | 2.34 | 14.8 |

### Example 18. Anti-Proliferation Activity of EGFR×cMET×cMET Trispecific Antibodies on EGFR or cMET Signaling-Dependent Cancer Cell Lines

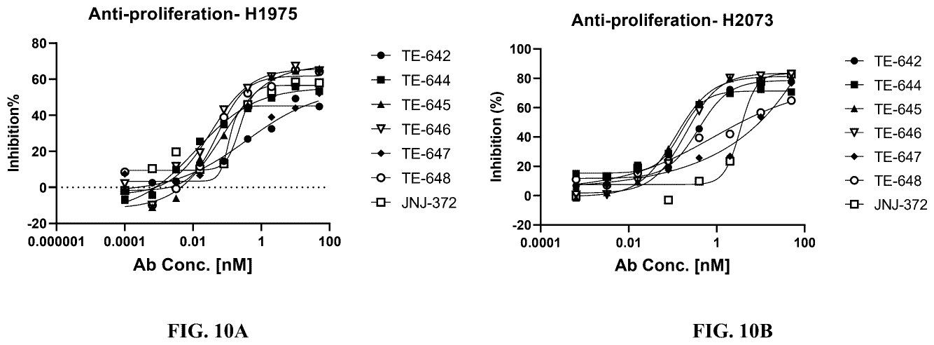

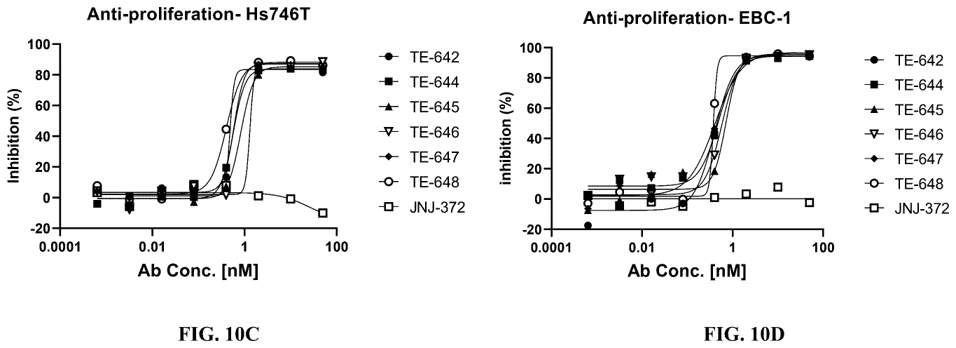

Mechanistically, EGFR×cMET×cMET trispecific antibodies inhibit either EGFR or c-MET signaling by down-regulating these kinase receptors on the cell surface, by which they inhibit proliferation on both c-MET and EGFR signaling-dependent cell lines. Cell-Titer-Glo assay (Promega) was used to evaluate the proliferation inhibition activities of these trispecific antibodies on both types of cell lines. Non-small cell lung cancer cell lines that depend on EGFR signaling (ligand independent): H1975 and H2073, and cell lines that depend on c-MET signaling (ligand independent): Hs746t and EBC-1 (human squamous lung cancer cell line) were selected as models for the evaluation. Briefly, cells were seeded to 96-well assay plates, treated with a series of dilutions of antibodies, and incubated at 37° C., 5% C02 for 6 days. After the treatment, 50 μl of Cell-Titer-Glo reagent was added in each well. The plates were shaken on an orbital shaker for 10 min to allow cell lysis. Luminescent signals were measured using BMG PheraStar with the luminescent protocol. IC₅₀ values were determined by fitting the dose-response data to the four-parameter logistic model with GraphPad Prism. As shown in Table 26 and FIGS. 10A-10B, all trispecific antibodies exhibited comparable or better anti-proliferation activity on EGFR signaling-dependent cell lines (H1975 and H2073) compared to JNJ-372. Strikingly, as shown in Table 26 and FIGS. 10C-10D, all trispecific antibodies exhibited strong anti-proliferation activity on c-MET signaling-driven cell lines (Hs746t and EBC-1), while JNJ-372 did not showed such activity.

TABLE 26
Anti-proliferation activity of trispecific antibodies on EGFR or c-MET
signaling-dependent cancer cell lines

| | EGFR signaling-dependent cells | | | | c-MET signaling-dependent cell | | | |
|---|---|---|---|---|---|---|---|---|
| | H1975 | | H2073 | | Hs746t | | EBC-1 | |
| Antibody | IC₅₀ (nM) | Iₘₐₓ (%) | IC₅₀ (nM) | Iₘₐₓ (%) | IC₅₀ (nM) | Iₘₐₓ (%) | IC₅₀ (nM) | Iₘₐₓ (%) |
| TE-642 | 0.18 | 49.26 | 0.38 | 77.76 | 0.47 | 84.86 | 0.4 | 95.24 |
| TE-644 | 0.02 | 55.93 | 0.16 | 72.47 | 0.57 | 83.93 | 0.49 | 94.47 |
| TE-645 | 0.07 | 65.84 | 0.12 | 80.36 | 0.83 | 85.56 | 0.72 | 94.62 |
| TE-646 | 0.03 | 67.33 | 0.2 | 82.81 | 1.31 | 88.6 | 0.58 | 95.39 |
| TE-647 | 0.11 | 52.24 | 4.89 | 76.73 | 0.59 | 87.98 | 0.41 | 95.28 |
| TE-648 | 0.05 | 65.12 | 0.42 | 64.89 | 0.4 | 89.17 | 0.38 | 95.95 |
| JNJ-372 | 0.23 | 58.44 | 3.46 | 82.99 | N/A | 0 | N/A | 0 |

### Example 19. Anti-Proliferation Activity of EGFR×cMET×cMET Trispecific Antibodies on Human Epidermal Keratinocytes, Neonatal (HEKn)

The skin-related adverse event is a typical on-target effect caused by hitting WT EGFR in skin cells, specifically, epidermal keratinocytes. To assess the skin toxicity risk of EGFR×cMET×cMET trispecific antibodies on normal keratinocytes, an anti-proliferation assay was performed on HEKn cells. The clinically approved drug JNJ-372 didn't exhibit serious skin-related risks in patients. The clinic stage LY3164530 led to a substantial percentage of skin-related adverse events in the phase I clinical trial, including maculopapular rash/dermatitis acneiform (83%, Grade 3/4 17%)¹¹. Both antibodies were internally generated and used as controls in the assay. Experimentally, HEKn cells were seeded to a 96-well assay plate, cells were treated with a series of dilutions of antibodies and incubated at 37° C., 5% CO₂ for 6 days. After treatment, 50 μl of Cell-Titer-Glo reagent (Promega) was added to each well. The plate was shaken on an orbital shaker for 10 min to allow cell lysis. The luminescent signal was measured using BMG PheraStar with the luminescent protocol. IC₅₀ values were determined by fitting the dose-response data to the four-parameter logistic model with GraphPad Prism. As shown in Table 27 and FIG. 11, TE-642, TE-644, TE-647 and TE-648 exhibited weaker anti-proliferation activity on HEKn cells than LY3164530. TE-647 exhibited a comparable potency (IC₅₀) and lower efficacy (Iₘₐₓ) than JNJ-372, which infers less toxicity on normal human keratinocytes than JNJ-372.

TABLE 27
Anti-proliferation activity of trispecific antibodies on HEKn

| | IC₅₀ | |
|---|---|---|
| Antibody | (nM) | Iₘₐₓ(%) |
| TE-642 | 1.84 | 28.36 |
| TE-644 | 0.71 | 26.74 |
| TE-645 | 0.25 | 41.94 |
| TE-646 | 0.43 | 42.26 |
| TE-647 | 4.58 | 22.22 |
| TE-648 | 1 | 12.84 |
| JNJ-372 | 5.7 | 33.53 |
| LY3164530 | 0.24 | 46.16 |

### Example 20. EGFR×cMET×cMET Trispecific Antibodies TE-642, TE-646 and TE-647 are Active In Vivo Against Human Lung Tumor Xenografts

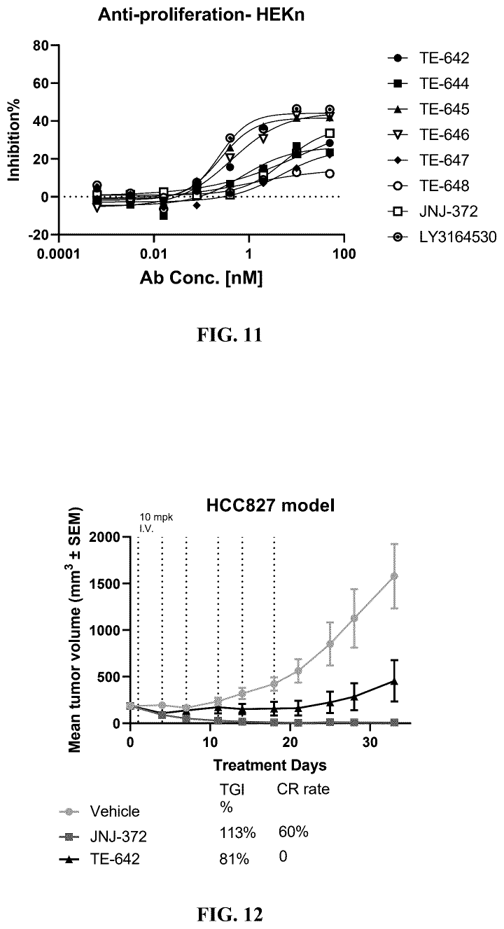

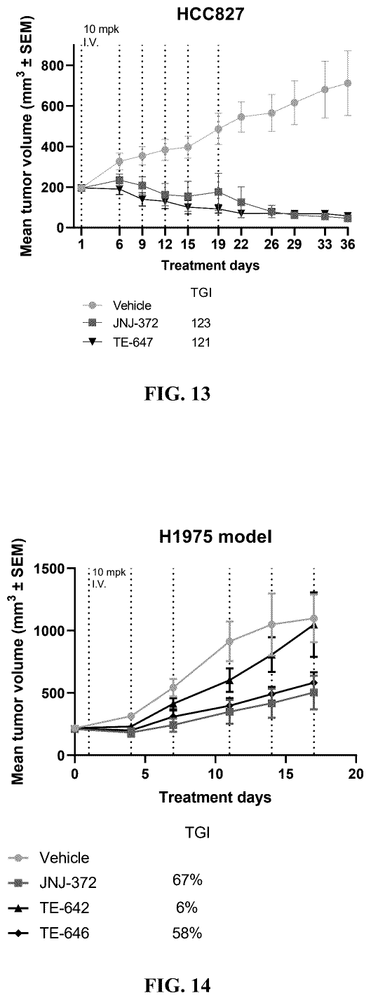

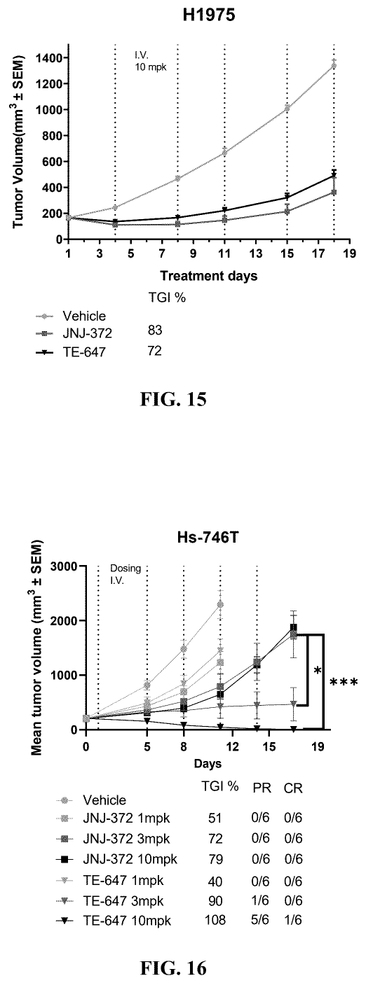

The in vivo efficacy of TE-642, TE-646, TE-647 and JNJ-372 were evaluated in mice transplanted with HCC827 (lung adenocarcinoma cell line with EGFR exon 19 deletion (E19del)) (FIG. 12 and FIG. 13) and H1975 (with EGFR L858R/T790M and without cMET amplification) (FIG. 14 and FIG. 15) human lung cancer cells.

Female BALB/c nude mice were subcutaneously implanted with 5×10⁶ HCC827 or H1975 cells (EGFR mutation driven model with EGFR L858R/T790M and without cMET amplification) per 200 μL PBS with 50% Matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 200-250 mm³ in size, mice with HCC827 or H1975 tumors were randomly allocated into 3 groups with 6 or 8 animals in each group, and were intravenously treated with vehicle, JNJ-372 or trispecific antibody (TE-647, TE-646, or TE-642) at 10 mg/kg biweekly (BIW) for 3 weeks. Partial regression (PR) was defined as tumor volume smaller than 50% of the starting tumor volume measured on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

In xenografts without c-MET amplification (EGFR mutation driven model, HCC827 model and H1975 model), TE-642 showed lower TGI (81% vs 113%) and lower CR rate (0 vs 60%) in HCC827 model (as shown in FIG. 12) and lower TGI (6% vs 67%) in H1975 model (as shown in FIG. 14), compared with JNJ-372.

TE-646 is comparable to JNJ-372 in tumor growth inhibition in xenografts without c-MET amplification (EGFR mutation driven model, H1975 model), as shown in FIG. 14.

TE-647 is comparable to JNJ-372 in tumor growth inhibition in xenografts without c-MET amplification (EGFR mutation driven model), i.e., in HCC827 model (as shown in FIG. 13) and in H1975 model (as shown in FIG. 15).

### Example 21. Trispecific Antibody TE-647 Inhibits Tumor Growth in Human Lung and Gastric Tumors in Animal Models

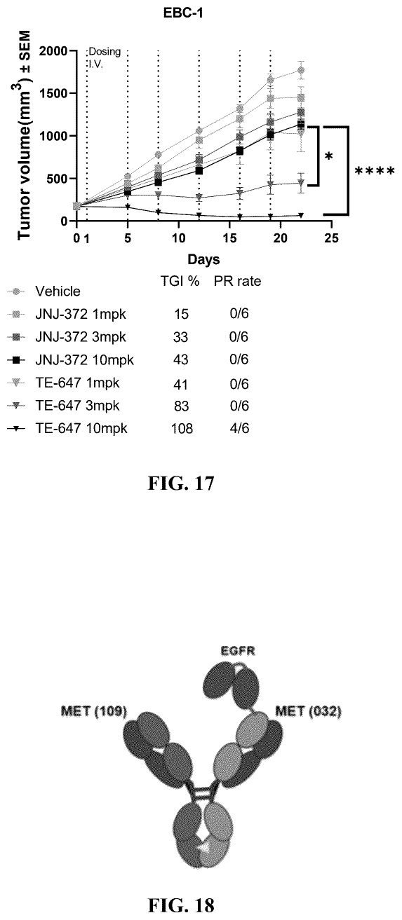

The in vivo efficacy of TE-647 and JNJ-372 were evaluated in mice transplanted with Hs746T (human gastric cancer cell, FIG. 16) and EBC-1 (human squamous lung cancer cell, FIG. 17).

Female BALB/c nude mice were subcutaneously implanted with 5×10⁶ Hs756T or EBC-1 (cMET amplification driven model without EGFR mutation) per 200 μL PBS with 50% Matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 200-250 mm³ in size, mice with Hs746T or EBC-1 tumors were randomly allocated into 7 groups with 6 animals in each group, and were intravenously treated with vehicle, JNJ-372 or TE-647 at 1, 3, 10 mg/kg BIW for 3 weeks. Partial regression (PR) was defined as tumor volume smaller than 50% of the starting tumor volume measured on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

Trispecific antibody TE-647 has shown robust and reproducible antitumor effects in human gastric and lung tumor xenografts (cMET amplification driven model). In xenografts with c-MET amplification (Hs746T and EBC-1), TE-647 showed better tumor growth inhibition than JNJ-372 and the effect is dose-dependent (FIG. 16 and FIG. 17), indicating the superior anti-tumor activity of TE-647 in models with c-MET amplification.

### Example 22. Physiochemical Properties of TE-647

### Hydrophobicity Assessment

To determine the hydrophobicity of TE-647 using HIC, 50 μg of sample at 1 mg/ml was diluted with a mobile phase A solution (1.5 M ammonium sulfate, 50 mM sodium phosphate, pH 7.0) to achieve a final ammonium sulfate concentration of about 1M before analysis. A MABPac HIC-10 column was used with a liner gradient of mobile phase A and mobile phase B solution (50 mM sodium phosphate, pH 7.0) over 29 min at a flow rate of 0.5 mg/min. Peak retention times were monitored at A280 absorbance. The results were summarized in Table 28. TE-647 showed no hydrophobicity risk.

### Thermostability Assessment (Tm and Tagg)

Fluorescence coupled with static light scattering was used to evaluate the thermal unfolding transition midpoint Tm (° C.) and the onset temperature of aggregation (Tagg). A UNit® (Unchained labs, Pleasanton, CA) system was used to measure the fluorescence and static light scattering simultaneously. During the measurement, 9 μL protein sample at 1 mg/mL was loaded to the cuvette; the samples were held at 20° C. for 120 s and then ramped to 95° C. at the rate of 0.3° C./min. Both fluorescence and static light scattering (at 266 nm) were collected after excitation at 266 nm. After measurement, the data were loaded onto Unit analysis software. Tm was gotten from the fluorescence and the Tagg was gotten from the static light scattering. The results were summarized in Table 28. Tm and Tagg of TE-647 were 67.3° C. and 60.0° C. respectively.

### Self-Association Assessment

The AC-SINS assay measures protein self-interaction by capturing the mAb on the surface of a gold colloid that displays surface resonance oscillations in frequency with visible light. As the immobilized antibodies self-interact, the colloids aggregate, changing the oscillation frequency to absorb at a longer wavelength. Gold nanoparticles were incubated with an 80/20 (v/v) capture antibody/non-capture antibody mixture. The coated gold nanoparticles were then spun down and resuspended into PBS. The samples were diluted to 0.05 mg/ml into the conjugation buffers and 45 μl of each dilution was loaded onto a 384-well plate. Five μl of previously prepared gold nanoparticles were then added to each well of the plate including mAbs and buffer controls. The plate was then covered with an aluminum lid, incubated at room temperature for 2 hours, and quickly spun down at 3000 rpm prior to reading the absorbance spectra of each well from 450 to 650 nm using a plate reader. Each sample spectra were recorded and analyzed for red-shifting of the maximum of absorption peak compared to buffers and mAb controls, the red-shifting and its strength is indicative of the self-interaction propensity of the tested mAb sample. The results were summarized in Table 28. The wavelength shift was 4.8 nm, which showed no self-association risk.

TABLE 28
Biophysical properties of TE-647

| | Retention Time | | | AC-SINS |
|---|---|---|---|---|
| Sample Name | (min) | Tm (° C.) | Tagg (° C.) | (Δλ) |
| TE-647 | 13.7 | 67.3 | 60.0 | 4.8 |

### Example 23: Generation of Anti-EGFR×cMET×cMET Trispecific Antibodies

A common light chain-based anti-cMET biparatopic antibody was screened, engineered, and assembled by KIH mutations (T366W for the knob in the heavy chain of arm 032 and T366S/L368A/Y407V for the hole in the heavy chain of arm 109) in the Fc region as described in PCT Patent Application Nos. PCT/CN2023/073074, PCT/CN2023/103335, and PCT/CN2024/072952, the contents of all of which are hereby incorporated in their entireties. The full-length human cMET and EGFR antibody sequences upon which the antigens for mouse immunization were based, as described in the foregoing patent applications, are provided in Table 29. A single valent EGFR scFv was fused to the N-terminus of one of the anti-cMET Fab (arm 032) in the heavy chains to form an anti-EGFR×cMET×cMET trispecific antibody as illustrated as FIG. 18

(T187 NE+wt IgG1, SEQ ID NOs: 148, 150, 153, 155-160, 56-58, 6, 7, 92, 66-68, 142-145, 64, and 175-179). To further improve the developability and pharmacokinetic properties, the trispecific antibody was further engineered in the variable and/or constant regions. Specifically, to remove the deamination site, N97F or N97H (Kabat numbering) was introduced in the VH of the anti-cMET Fab (arm 109) for some variants (i.e., T187 FE+wt IgG1, SEQ ID NOs: 153, 170, 148, 155-160, 56-58, 6, 7, 90, 66-68, 142, 143, 145, 173, 64, 175-177, 179, and 180; T187 HE+wt IgG1, SEQ ID NOs: 153, 172, 148, 155-160, 56-58, 6, 7, 88, 66-68, 142, 143, 145, 174, 64175-177, 179, and 181; and T187 FE+YTE, SEQ ID NOs: 168, 171, 148, 155-160, 56-58, 6, 7, 90, 66-68, 142, 143, 145, 173, 64, 175-177, 179, and 180). Meanwhile, M252Y/S254T/T256E (EU numbering, YTE mutation) in the Fc region were introduced to decrease the non-specific clearance of the antibodies (T187 NE+YTE, SEQ ID NOs: 168, 169, 148, 155-160, 56-58, 6, 7, 92, 66-68, 142-145, 64, and 175-179; and T187 FE+YTE, SEQ ID NOs: 168, 171, 148, 155-160, 56-58, 6, 7, 90, 66-68, 142, 143, 145, 173, 64, 175-177, 179, and 180). The sequences of these antibodies are summarized in Tables 30 and 31.

TABLE 29
cMET and EGFR Sequences

| | SEQ ID | | |
|---|---|---|---|
| Antigen | NO: | Description | SEQUENCE |
| Human | 69 | Full-length | MKAPAVLAPGILVLLFTLVQRSNGECKEALAKSEMNVNMKY |
| cMET | | AA | QLPNFTAETPIQNVILHEHHIFLGATNYIYVLNEEDLQKVAEY |
| | | sequence | KTGPVLEHPDCFPCQDCSSKANLSGGVWKDNINMALVVDTY |
| | | | YDDQLISCGSVNRGTCQRHVFPHNHTADIQSEVHCIFSPQIEE |
| | | | PSQCPDCVVSALGAKVLSSVKDRFINFFVGNTINSSYFPDHPL |
| | | | HSISVRRLKETKDGFMFLTDQSYIDVLPEFRDSYPIKYVHAFE |
| | | | SNNFIYFLTVQRETLDAQTFHTRIIRFCSINSGLHSYMEMPLEC |
| | | | ILTEKRKKRSTKKEVFNILQAAYVSKPGAQLARQIGASLNDDI |
| | | | LFGVFAQSKPDSAEPMDRSAMCAFPIKYVNDFFNKIVNKNN |
| | | | VRCLQHFYGPNHEHCFNRTLLRNSSGCEARRDEYRTEFTTAL |
| | | | QRVDLFMGQFSEVLLTSISTFIKGDLTIANLGTSEGRFMQVVV |
| | | | SRSGPSTPHVNFLLDSHPVSPEVIVEHTLNQNGYTLVITGKKIT |
| | | | KIPLNGLGCRHFQSCSQCLSAPPFVQCGWCHDKCVRSEECLS |
| | | | GTWTQQICLPAIYKVFPNSAPLEGGTRLTICGWDFGFRRNNK |
| | | | FDLKKTRVLLGNESCTLTLSESTMNTLKCTVGPAMNKHFNM |
| | | | SIIISNGHGTTQYSTFSYVDPVITSISPKYGPMAGGTLLTLTGN |
| | | | YLNSGNSRHISIGGKTCTLKSVSNSILECYTPAQTISTEFAVKL |
| | | | KIDLANRETSIFSYREDPIVYEIHPTKSFISGGSTITGVGKNLNS |
| | | | VSVPRMVINVHEAGRNFTVACQHRSNSEIICCTTPSLQQLNLQ |
| | | | LPLKTKAFFMLDGILSKYFDLIYVHNPVFKPFEKPVMISMGN |
| | | | ENVLEIKGNDIDPEAVKGEVLKVGNKSCENIHLHSEAVLCTVP |
| | | | NDLLKLNSELNIEWKQAISSTVLGKVIVQPDQNFTGLIAGVVS |
| | | | ISTALLLLLGFFLWLKKRKQIKDLGSELVRYDARVHTPHLDRL |
| | | | VSARSVSPTTEMVSNESVDYRATFPEDQFPNSSQNGSCRQVQ |
| | | | YPLTDMSPILTSGDSDISSPLLQNTVHIDLSALNPELVQAVQHV |
| | | | VIGPSSLIVHFNEVIGRGHFGCVYHGTLLDNDGKKIHCAVKSL |
| | | | NRITDIGEVSQFLTEGIIMKDFSHPNVLSLLGICLRSEGSPLVVL |
| | | | PYMKHGDLRNFIRNETHNPTVKDLIGFGLQVAKGMKYLASK |
| | | | KFVHRDLAARNCMLDEKFTVKVADFGLARDMYDKEYYSVH |
| | | | NKTGAKLPVKWMALESLQTQKFTTKSDVWSFGVLLWELMT |
| | | | RGAPPYPDVNTFDITVYLLQGRRLLQPEYCPDPLYEVMLKC |
| | | | WHPKAEMRPSFSELVSRISAIFSTFIGEHYVHVNATYVNVKCV |
| | | | APYPSLLSSEDNADDEVDTRPASFWETS |
| Human | 167 | Full-length | MRPSGTAGAALLALLAALCPASRALEEKKVCQGTSNKLTQL |
| EGFR | | AA | GTFEDHFLSLQRMFNNCEVVLGNLEITYVQRNYDLSFLKTIQ |
| | | sequence | EVAGYVLIALNTVERIPLENLQIIRGNMYYENSYALAVLSNYD |
| | | | ANKTGLKELPMRNLQEILHGAVRFSNNPALCNVESIQWRDIV |
| | | | SSDFLSNMSMDFQNHLGSCQKCDPSCPNGSCWGAGEENCQK |
| | | | LTKIICAQQCSGRCRGKSPSDCCHNQCAAGCTGPRESDCLVC |
| | | | RKFRDEATCKDTCPPLMLYNPTTYQMDVNPEGKYSFGATCV |
| | | | KKCPRNYVVTDHGSCVRACGADSYEMEEDGVRKCKKCEGP |
| | | | CRKVCNGIGIGEFKDSLSINATNIKHFKNCTSISGDLHILPVAF |
| | | | RGDSFTHTPPLDPQELDILKTVKEITGFLLIQAWPENRTDLHA |
| | | | FENLEIIRGRTKQHGQFSLAVVSLNITSLGLRSLKEISDGDVIIS |
| | | | GNKNLCYANTINWKKLFGTSGQKTKIISNRGENSCKATGQVC |
| | | | HALCSPEGCWGPEPRDCVSCRNVSRGRECVDKCNLLEGEPR |
| | | | EFVENSECIQCHPECLPQAMNITCTGRGPDNCIQCAHYIDGPH |
| | | | CVKTCPAGVMGENNTLVWKYADAGHVCHLCHPNCTYGCTG |
| | | | PGLEGCPTNGPKIPSIATGMVGALLLLLVVALGIGLFMRRRHI |
| | | | VRKRTLRRLLQERELVEPLTPSGEAPNQALLRILKETEFKKIK |
| | | | VLGSGAFGTVYKGLWIPEGEKVKIPVAIKELREATSPKANKEI |
| | | | LDEAYVMASVDNPHVCRLLGICLTSTVQLITQLMPFGCLLDY |
| | | | VREHKDNIGSQYLLNWCVQIAKGMNYLEDRRLVHRDLAAR |
| | | | NVLVKTPQHVKITDFGLAKLLGAEEKEYHAEGGKVPIKWMA |
| | | | LESILHRIYTHQSDVWSYGVTVWELMTFGSKPYDGIPASEISSI |
| | | | LEKGERLPQPPICTIDVYMIMVKCWMIDADSRPKFRELIIEFS |
| | | | KMARDPQRYLVIQGDERMHLPSPTDSNFYRALMDEEDMDD |
| | | | VVDADEYLIPQQGFFSSPSTSRTPLLSSLSATSNNSTVACIDRN |
| | | | GLQSCPIKEDSFLQRYSSDPTGALTEDSIDDTFLPVPEYINQSV |
| | | | PKRPAGSVQNPVYHNQPLNPAPSRDPHYQDPHSTAVGNPEYL |
| | | | NTVQPTCVNSTFDSPAHWAQKGSHQISLDNPDYQQDFFPKEA |
| | | | KPNGIFKGSTAENAEYLRVAPQSSEFIGA |

TABLE 30
Antibody Amino Acid Sequences

| | SEQ ID | | |
|---|---|---|---|
| Antibody | NO: | Description | SEQUENCE |
| T187 (NE | 153 | HC-knob | PKLLIYDASSLESGVPSRFSGSESGTDFTLTISSLQPEDFATYY |
| + wt | | (zalu scFv + | CQQFNSYPLTFGGGTKVEIKGGGGSGGGGSGGGGSGGGGSQ |
| IgG1) | | arm 032- | VQLVESGGGVVQPGRSLRLSCAASGFTFSTYGMHWVRQAPG |
| BGX1 | | knob) | KGLEWVAVIWDDGSYKYYGDSVKGRFTISRDNSKNTLYLQ |
| | | | MNSLRAEDTAVYYCARDGITMVRGVMKDYFDYWGQGTLV |
| | | | TVSSGGGGSGGGGSGGGGSGGGGSEFQLQESGPGLVKPSETL |
| | | | RLTCTVSGGSISTSSYYWGWIRQPPGKGLEWIGTIYYSGTTY |
| | | | YNPSLKSRVTISVDTSKNQFSLKLKSVRAADTSIYYCARHWP |
| | | | KYNSWFDPWGQGILVTVSSASTKGPSVFPLAPSSKSTSGGTA |
| | | | ALGCLVKDYFPEPVTVSWNSGALTSGVHTFPAVLQSSGLYSL |
| | | | SSVVTVPSSSLGTQTYICNVNHKPSNTKVDKKVEPKSCDKTH |
| | | | TCPPCPAPELLGGPSVFLFPPKPKDTLMISRTPEVTCVVVDVS |
| | | | HEDPEVKFNWYVDGVEVHNAKTKPREEQYNSTYRVVSVLT |
| | | | VLHQDWLNGKEYKCKVSNKALPAPIEKTISKAKGQPREPQV |
| | | | YTLPPSREEMTKNQVSLWCLVKGFYPSDIAVEWESNGQPEN |
| | | | NYKTTPPVLDSDGSFFLYSKLTVDKSRWQQGNVFSCSVMHE |
| | | | ALHNHYTQKSLSLSPGK |
| | 150 | HC-hole | EVQLVESGGEVVQPGESLRLSCAASGFTFSSHGMHWVRQAP |
| | | (arm 109- | GKGLEWVAVISYDESDKYYADSVKGRFTISRDHSKNTLYLE |
| | | hole, NE) | MNSLRAEDTAVYYCVKDRNEGYSGWLDYWGQGTLVTVSS |
| | | | ASTKGPSVFPLAPSSKSTSGGTAALGCLVKDYFPEPVTVSWN |
| | | | SGALTSGVHTFPAVLQSSGLYSLSSVVTVPSSSLGTQTYICNV |
| | | | NHKPSNTKVDKKVEPKSCDKTHTCPPCPAPELLGGPSVFLFP |
| | | | PKPKDTLMISRTPEVTCVVVDVSHEDPEVKFNWYVDGVEVH |
| | | | NAKTKPREEQYNSTYRVVSVLTVLHQDWLNGKEYKCKVSN |
| | | | KALPAPIEKTISKAKGQPREPQVYTLPPSREEMTKNQVSLSCA |
| | | | VKGFYPSDIAVEWESNGQPENNYKTTPPVLDSDGSFFLVSKL |
| | | | TVDKSRWQQGNVFSCSVMHEALHNHYTQKSLSLSPGK |
| | 148 | LC | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | 158 | ScFv- | RASQDISSALV |
| | | LCDR1 | |
| | | (Kabat) | |
| | 159 | ScFv- | DASSLES |
| | | LCDR2 | |
| | | (Kabat) | |
| | 160 | scFv- | QQFNSYPLT |
| | | LCDR3 | |
| | | (Kabat) | |
| | 155 | ScFv- | TYGMH |
| | | HCDR1 | |
| | | (Kabat) | |
| | 156 | ScFv- | VIWDDGSYKYYGDSVKG |
| | | HCDR2 | |
| | | (Kabat) | |
| | 157 | ScFv- | DGITMVRGVMKDYFDY |
| | | HCDR3 | |
| | | (Kabat) | |
| | 56 | arm 032- | TSSYYWG |
| | | HCDR1 | |
| | | (Kabat) | |
| | 57 | arm 032- | TIYYSGTTYYNPSLKS |
| | | HCDR2 | |
| | | (Kabat) | |
| | 58 | arm 032- | HWPKYNSWFDP |
| | | HCDR3 | |
| | | (Kabat) | |
| | 6 | arm 109- | SHGMH |
| | | HCDR1 | |
| | | (Kabat) | |
| | 7 | arm 109- | VISYDESDKYYADSVKG |
| | | HCDR2 | |
| | | (Kabat) | |
| | 92 | arm 109-NE- | DRNEGYSGWLDY |
| | | HCDR3 | |
| | | (Kabat) | |
| | 66 | LCDR1 | RASQSVSSYLA |
| | | (Kabat) | |
| | 67 | LCDR2 | DASNRAT |
| | | (Kabat) | |
| | 68 | LCDR3 | QQRSNWPPT |
| | | (Kabat) | |
| | 142 | Zalu VH | QVQLVESGGGVVQPGRSLRLSCAASGFTFSTYGMHWVRQAP |
| | | | GKGLEWVAVIWDDGSYKYYGDSVKGRFTISRDNSKNTLYL |
| | | | QMNSLRAEDTAVYYCARDGITMVRGVMKDYFDYWGQGTL |
| | | | VTVSS |
| | 143 | Zalu VL | AIQLTQSPSSLSASVGDRVTITCRASQDISSALVWYQQKPGKA |
| | | | PKLLIYDASSLESGVPSRFSGSESGTDFTLTISSLQPEDFATYY |
| | | | CQQFNSYPLTFGGGTKVEIK |
| | 145 | arm 032 VH | EFQLQESGPGLVKPSETLRLTCTVSGGSISTSSYYWGWIRQPP |
| | | | GKGLEWIGTIYYSGTTYYNPSLKSRVTISVDTSKNQFSLKLKS |
| | | | VRAADTSIYYCARHWPKYNSWFDPWGQGILVTVSS |
| | 144 | arm 109- | EVQLVESGGEVVQPGESLRLSCAASGFTFSSHGMHWVRQAP |
| | | NE VH | GKGLEWVAVISYDESDKYYADSVKGRFTISRDHSKNTLYLE |
| | | | MNSLRAEDTAVYYCVKDRNEGYSGWLDYWGQGTLVTVSS |
| | 64 | VL | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIK |
| T187 (NE | 168 | HC-knob- | AIQLTQSPSSLSASVGDRVTITCRASQDISSALVWYQQKPGKA |
| + IgG1- | | YTE (zalu | PKLLIYDASSLESGVPSRFSGSESGTDFTLTISSLQPEDFATYY |
| YTE) | | scFv + arm | CQQFNSYPLTFGGGTKVEIKGGGGSGGGGSGGGGSGGGGSQ |
| BGX5 | | 032-knob- | VQLVESGGGVVQPGRSLRLSCAASGFTFSTYGMHWVRQAPG |
| | | YTE) | KGLEWVAVIWDDGSYKYYGDSVKGRFTISRDNSKNTLYLQ |
| | | | MNSLRAEDTAVYYCARDGITMVRGVMKDYFDYWGQGTLV |
| | | | TVSSGGGGSGGGGSGGGGSGGGGSEFQLQESGPGLVKPSETL |
| | | | RLTCTVSGGSISTSSYYWGWIRQPPGKGLEWIGTIYYSGTTY |
| | | | YNPSLKSRVTISVDTSKNQFSLKLKSVRAADTSIYYCARHWP |
| | | | KYNSWFDPWGQGILVTVSSASTKGPSVFPLAPSSKSTSGGTA |
| | | | ALGCLVKDYFPEPVTVSWNSGALTSGVHTFPAVLQSSGLYSL |
| | | | SSVVTVPSSSLGTQTYICNVNHKPSNTKVDKKVEPKSCDKTH |
| | | | TCPPCPAPELLGGPSVFLFPPKPKDTLYITREPEVTCVVVDVS |
| | | | HEDPEVKFNWYVDGVEVHNAKTKPREEQYNSTYRVVSVLT |
| | | | VLHQDWLNGKEYKCKVSNKALPAPIEKTISKAKGQPREPQV |
| | | | YTLPPSREEMTKNQVSLWCLVKGFYPSDIAVEWESNGQPEN |
| | | | NYKTTPPVLDSDGSFFLYSKLTVDKSRWQQGNVFSCSVMHE |
| | | | ALHNHYTQKSLSLSPGK |
| | 169 | HC-hole- | EVQLVESGGEVVQPGESLRLSCAASGFTFSSHGMHWVRQAP |
| | | YTE (arm | GKGLEWVAVISYDESDKYYADSVKGRFTISRDHSKNTLYLE |
| | | 109-hole- | MNSLRAEDTAVYYCVKDRNEGYSGWLDYWGQGTLVTVSS |
| | | YTE, NE) | ASTKGPSVFPLAPSSKSTSGGTAALGCLVKDYFPEPVTVSWN |
| | | | SGALTSGVHTFPAVLQSSGLYSLSSVVTVPSSSLGTQTYICNV |
| | | | NHKPSNTKVDKKVEPKSCDKTHTCPPCPAPELLGGPSVFLFP |
| | | | PKPKDTLYITREPEVTCVVVDVSHEDPEVKFNWYVDGVEVH |
| | | | NAKTKPREEQYNSTYRVVSVLTVLHQDWLNGKEYKCKVSN |
| | | | KALPAPIEKTISKAKGQPREPQVYTLPPSREEMTKNQVSLSCA |
| | | | VKGFYPSDIAVEWESNGQPENNYKTTPPVLDSDGSFFLVSKL |
| | | | TVDKSRWQQGNVFSCSVMHEALHNHYTQKSLSLSPGK |
| | 148 | LC | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | 158 | ScFv- | RASQDISSALV |
| | | LCDR1 | |
| | | (Kabat) | |
| | 159 | ScFv- | DASSLES |
| | | LCDR2 | |
| | | (Kabat) | |
| | 160 | scFv- | QQFNSYPLT |
| | | LCDR3 | |
| | | (Kabat) | |
| | 155 | ScFv- | TYGMH |
| | | HCDR1 | |
| | | (Kabat) | |
| | 156 | ScFv- | VIWDDGSYKYYGDSVKG |
| | | HCDR2 | |
| | | (Kabat) | |
| | 157 | ScFv- | DGITMVRGVMKDYFDY |
| | | HCDR3 | |
| | | (Kabat) | |
| | 56 | arm 032- | TSSYYWG |
| | | HCDR1 | |
| | | (Kabat) | |
| | 57 | arm 032- | TIYYSGTTYYNPSLKS |
| | | HCDR2 | |
| | | (Kabat) | |
| | 58 | arm 032- | HWPKYNSWFDP |
| | | HCDR3 | |
| | | (Kabat) | |
| | 6 | arm 109- | SHGMH |
| | | HCDR1 | |
| | | (Kabat) | |
| | 7 | arm 109- | VISYDESDKYYADSVKG |
| | | HCDR2 | |
| | | (Kabat) | |
| | 92 | arm 109-NE- | DRNEGYSGWLDY |
| | | HCDR3 | |
| | | (Kabat) | |
| | 66 | LCDR1 | RASQSVSSYLA |
| | | (Kabat) | |
| | 67 | LCDR2 | DASNRAT |
| | | (Kabat) | |
| | 68 | LCDR3 | QQRSNWPPT |
| | | (Kabat) | |
| | 142 | Zalu VH | QVQLVESGGGVVQPGRSLRLSCAASGFTFSTYGMHWVRQAP |
| | | | GKGLEWVAVIWDDGSYKYYGDSVKGRFTISRDNSKNTLYL |
| | | | QMNSLRAEDTAVYYCARDGITMVRGVMKDYFDYWGQGTL |
| | | | VTVSS |
| | 143 | Zalu VL | AIQLTQSPSSLSASVGDRVTITCRASQDISSALVWYQQKPGKA |
| | | | PKLLIYDASSLESGVPSRFSGSESGTDFTLTISSLQPEDFATYY |
| | | | CQQFNSYPLTFGGGTKVEIK |
| | 145 | arm 032 VH | EFQLQESGPGLVKPSETLRLTCTVSGGSISTSSYYWGWIRQPP |
| | | | GKGLEWIGTIYYSGTTYYNPSLKSRVTISVDTSKNQFSLKLKS |
| | | | VRAADTSIYYCARHWPKYNSWFDPWGQGILVTVSS |
| | 144 | arm 109- | EVQLVESGGEVVQPGESLRLSCAASGFTFSSHGMHWVRQAP |
| | | NE VH | GKGLEWVAVISYDESDKYYADSVKGRFTISRDHSKNTLYLE |
| | | | MNSLRAEDTAVYYCVKDRNEGYSGWLDYWGQGTLVTVSS |
| | 64 | VL | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIK |
| T187 (FE | 153 | HC-knob | AIQLTQSPSSLSASVGDRVTITCRASQDISSALVWYQQKPGKA |
| + wt | | (Zalu scFv + | PKLLIYDASSLESGVPSRFSGSESGTDFTLTISSLQPEDFATYY |
| IgG1) | | arm 032- | CQQFNSYPLTFGGGTKVEIKGGGGSGGGGSGGGGSGGGGSQ |
| BGX2 | | knob) | VQLVESGGGVVQPGRSLRLSCAASGFTFSTYGMHWVRQAPG |
| | | | KGLEWVAVIWDDGSYKYYGDSVKGRFTISRDNSKNTLYLQ |
| | | | MNSLRAEDTAVYYCARDGITMVRGVMKDYFDYWGQGTLV |
| | | | TVSSGGGGSGGGGSGGGGSGGGGSEFQLQESGPGLVKPSETL |
| | | | RLTCTVSGGSISTSSYYWGWIRQPPGKGLEWIGTIYYSGTTY |
| | | | YNPSLKSRVTISVDTSKNQFSLKLKSVRAADTSIYYCARHWP |
| | | | KYNSWFDPWGQGILVTVSSASTKGPSVFPLAPSSKSTSGGTA |
| | | | ALGCLVKDYFPEPVTVSWNSGALTSGVHTFPAVLQSSGLYSL |
| | | | SSVVTVPSSSLGTQTYICNVNHKPSNTKVDKKVEPKSCDKTH |
| | | | TCPPCPAPELLGGPSVFLFPPKPKDTLMISRTPEVTCVVVDVS |
| | | | HEDPEVKFNWYVDGVEVHNAKTKPREEQYNSTYRVVSVLT |
| | | | VLHQDWLNGKEYKCKVSNKALPAPIEKTISKAKGQPREPQV |
| | | | YTLPPSREEMTKNQVSLWCLVKGFYPSDIAVEWESNGQPEN |
| | | | NYKTTPPVLDSDGSFFLYSKLTVDKSRWQQGNVFSCSVMHE |
| | | | ALHNHYTQKSLSLSPGK |
| | 170 | HC-hole | EVQLVESGGEVVQPGESLRLSCAASGFTFSSHGMHWVRQAP |
| | | (arm 109- | GKGLEWVAVISYDESDKYYADSVKGRFTISRDHSKNTLYLE |
| | | hole, FE) | MNSLRAEDTAVYYCVKDRFEGYSGWLDYWGQGTLVTVSSA |
| | | | STKGPSVFPLAPSSKSTSGGTAALGCLVKDYFPEPVTVSWNS |
| | | | GALTSGVHTFPAVLQSSGLYSLSSVVTVPSSSLGTQTYICNVN |
| | | | HKPSNTKVDKKVEPKSCDKTHTCPPCPAPELLGGPSVFLFPP |
| | | | KPKDTLMISRTPEVTCVVVDVSHEDPEVKFNWYVDGVEVHN |
| | | | AKTKPREEQYNSTYRVVSVLTVLHQDWLNGKEYKCKVSNK |
| | | | ALPAPIEKTISKAKGQPREPQVYTLPPSREEMTKNQVSLSCAV |
| | | | KGFYPSDIAVEWESNGQPENNYKTTPPVLDSDGSFFLVSKLT |
| | | | VDKSRWQQGNVFSCSVMHEALHNHYTQKSLSLSPGK |
| | 148 | LC | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | 158 | ScFv- | RASQDISSALV |
| | | LCDR1 | |
| | | (Kabat) | |
| | 159 | ScFv- | DASSLES |
| | | LCDR2 | |
| | | (Kabat) | |
| | 160 | scFv- | QQFNSYPLT |
| | | LCDR3 | |
| | | (Kabat) | |
| | 155 | ScFv- | TYGMH |
| | | HCDR1 | |
| | | (Kabat) | |
| | 156 | ScFv- | VIWDDGSYKYYGDSVKG |
| | | HCDR2 | |
| | | (Kabat) | |
| | 157 | ScFv- | DGITMVRGVMKDYFDY |
| | | HCDR3 | |
| | | (Kabat) | |
| | 56 | arm 032- | TSSYYWG |
| | | HCDR1 | |
| | | (Kabat) | |
| | 57 | arm 032- | TIYYSGTTYYNPSLKS |
| | | HCDR2 | |
| | | (Kabat) | |
| | 58 | arm 032- | HWPKYNSWFDP |
| | | HCDR3 | |
| | | (Kabat) | |
| | 6 | arm 109- | SHGMH |
| | | HCDR1 | |
| | | (Kabat) | |
| | 7 | arm 109- | VISYDESDKYYADSVKG |
| | | HCDR2 | |
| | | (Kabat)) | |
| | 90 | arm 109- | DRFEGYSGWLDY |
| | | FE-HCDR3 | |
| | | (Kabat) | |
| | 66 | LCDR1 | RASQSVSSYLA |
| | | (Kabat) | |
| | 67 | LCDR2 | DASNRAT |
| | | (Kabat) | |
| | 68 | LCDR3 | QQRSNWPPT |
| | | (Kabat) | |
| | 142 | Zalu VH | QVQLVESGGGVVQPGRSLRLSCAASGFTFSTYGMHWVRQAP |
| | | | GKGLEWVAVIWDDGSYKYYGDSVKGRFTISRDNSKNTLYL |
| | | | QMNSLRAEDTAVYYCARDGITMVRGVMKDYFDYWGQGTL |
| | | | VTVSS |
| | 143 | Zalu VL | AIQLTQSPSSLSASVGDRVTITCRASQDISSALVWYQQKPGKA |
| | | | PKLLIYDASSLESGVPSRFSGSESGTDFTLTISSLQPEDFATYY |
| | | | CQQFNSYPLTFGGGTKVEIK |
| | 145 | arm 032 VH | EFQLQESGPGLVKPSETLRLTCTVSGGSISTSSYYWGWIRQPP |
| | | | GKGLEWIGTIYYSGTTYYNPSLKSRVTISVDTSKNQFSLKLKS |
| | | | VRAADTSIYYCARHWPKYNSWFDPWGQGILVTVSS |
| | 173 | arm 109- | EVQLVESGGEVVQPGESLRLSCAASGFTFSSHGMHWVRQAP |
| | | FE VH | GKGLEWVAVISYDESDKYYADSVKGRFTISRDHSKNTLYLE |
| | | | MNSLRAEDTAVYYCVKDRFEGYSGWLDYWGQGTLVTVSS |
| | 64 | VL | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIK |
| T187 (FE | 168 | HC-knob- | AIQLTQSPSSLSASVGDRVTITCRASQDISSALVWYQQKPGKA |
| + IgG1- | | YTE (zalu | PKLLIYDASSLESGVPSRFSGSESGTDFTLTISSLQPEDFATYY |
| YTE) | | scFv + arm | CQQFNSYPLTFGGGTKVEIKGGGGSGGGGSGGGGSGGGGSQ |
| BGX4 | | 032-knob- | VQLVESGGGVVQPGRSLRLSCAASGFTFSTYGMHWVRQAPG |
| | | YTE) | KGLEWVAVIWDDGSYKYYGDSVKGRFTISRDNSKNTLYLQ |
| | | | MNSLRAEDTAVYYCARDGITMVRGVMKDYFDYWGQGTLV |
| | | | TVSSGGGGSGGGGSGGGGSGGGGSEFQLQESGPGLVKPSETL |
| | | | RLTCTVSGGSISTSSYYWGWIRQPPGKGLEWIGTIYYSGTTY |
| | | | YNPSLKSRVTISVDTSKNQFSLKLKSVRAADTSIYYCARHWP |
| | | | KYNSWFDPWGQGILVTVSSASTKGPSVFPLAPSSKSTSGGTA |
| | | | ALGCLVKDYFPEPVTVSWNSGALTSGVHTFPAVLQSSGLYSL |
| | | | SSVVTVPSSSLGTQTYICNVNHKPSNTKVDKKVEPKSCDKTH |
| | | | TCPPCPAPELLGGPSVFLFPPKPKDTLYITREPEVTCVVVDVS |
| | | | HEDPEVKFNWYVDGVEVHNAKTKPREEQYNSTYRVVSVLT |
| | | | VLHQDWLNGKEYKCKVSNKALPAPIEKTISKAKGQPREPQV |
| | | | YTLPPSREEMTKNQVSLWCLVKGFYPSDIAVEWESNGQPEN |
| | | | NYKTTPPVLDSDGSFFLYSKLTVDKSRWQQGNVFSCSVMHE |
| | | | ALHNHYTQKSLSLSPGK |
| | 171 | HC-hole- | EVQLVESGGEVVQPGESLRLSCAASGFTFSSHGMHWVRQAP |
| | | YTE (arm | GKGLEWVAVISYDESDKYYADSVKGRFTISRDHSKNTLYLE |
| | | 109-hole- | MNSLRAEDTAVYYCVKDRFEGYSGWLDYWGQGTLVTVSSA |
| | | YTE, FE) | STKGPSVFPLAPSSKSTSGGTAALGCLVKDYFPEPVTVSWNS |
| | | | GALTSGVHTFPAVLQSSGLYSLSSVVTVPSSSLGTQTYICNVN |
| | | | HKPSNTKVDKKVEPKSCDKTHTCPPCPAPELLGGPSVFLFPP |
| | | | KPKDTLYITREPEVTCVVVDVSHEDPEVKFNWYVDGVEVHN |
| | | | AKTKPREEQYNSTYRVVSVLTVLHQDWLNGKEYKCKVSNK |
| | | | ALPAPIEKTISKAKGQPREPQVYTLPPSREEMTKNQVSLSCAV |
| | | | KGFYPSDIAVEWESNGQPENNYKTTPPVLDSDGSFFLVSKLT |
| | | | VDKSRWQQGNVFSCSVMHEALHNHYTQKSLSLSPGK |
| | 148 | LC | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | 158 | ScFv- | RASQDISSALV |
| | | LCDR1 | |
| | | (Kabat) | |
| | 159 | ScFv- | DASSLES |
| | | LCDR2 | |
| | | (Kabat) | |
| | 160 | scFv- | QQFNSYPLT |
| | | LCDR3 | |
| | | (Kabat) | |
| | 155 | ScFv- | TYGMH |
| | | HCDR1 | |
| | | (Kabat) | |
| | 156 | ScFv- | VIWDDGSYKYYGDSVKG |
| | | HCDR2 | |
| | | (Kabat) | |
| | 157 | ScFv- | DGITMVRGVMKDYFDY |
| | | HCDR3 | |
| | | (Kabat) | |
| | 56 | arm 032- | TSSYYWG |
| | | HCDR1 | |
| | | (Kabat) | |
| | 57 | arm 032- | TIYYSGTTYYNPSLKS |
| | | HCDR2 | |
| | | (Kabat) | |
| | 58 | arm 032- | HWPKYNSWFDP |
| | | HCDR3 | |
| | | (Kabat) | |
| | 6 | arm 109- | SHGMH |
| | | HCDR1 | |
| | | (Kabat) | |
| | 7 | arm 109- | VISYDESDKYYADSVKG |
| | | HCDR2 | |
| | | (Kabat)) | |
| | 90 | arm 109- | DRFEGYSGWLDY |
| | | FE-HCDR3 | |
| | | (Kabat) | |
| | 66 | LCDR1 | RASQSVSSYLA |
| | | (Kabat) | |
| | 67 | LCDR2 | DASNRAT |
| | | (Kabat) | |
| | 68 | LCDR3 | QQRSNWPPT |
| | | (Kabat) | |
| | 142 | Zalu VH | QVQLVESGGGVVQPGRSLRLSCAASGFTFSTYGMHWVRQAP |
| | | | GKGLEWVAVIWDDGSYKYYGDSVKGRFTISRDNSKNTLYL |
| | | | QMNSLRAEDTAVYYCARDGITMVRGVMKDYFDYWGQGTL |
| | | | VTVSS |
| | 143 | Zalu VL | AIQLTQSPSSLSASVGDRVTITCRASQDISSALVWYQQKPGKA |
| | | | PKLLIYDASSLESGVPSRFSGSESGTDFTLTISSLQPEDFATYY |
| | | | CQQFNSYPLTFGGGTKVEIK |
| | 145 | 032 VH | EFQLQESGPGLVKPSETLRLTCTVSGGSISTSSYYWGWIRQPP |
| | | | GKGLEWIGTIYYSGTTYYNPSLKSRVTISVDTSKNQFSLKLKS |
| | | | VRAADTSIYYCARHWPKYNSWFDPWGQGILVTVSS |
| | 173 | arm 109- | EVQLVESGGEVVQPGESLRLSCAASGFTFSSHGMHWVRQAP |
| | | FE VH | GKGLEWVAVISYDESDKYYADSVKGRFTISRDHSKNTLYLE |
| | | | MNSLRAEDTAVYYCVKDRFEGYSGWLDYWGQGTLVTVSS |
| | 64 | VL | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIK |
| T187 (HE | 153 | HC-knob | AIQLTQSPSSLSASVGDRVTITCRASQDISSALVWYQQKPGKA |
| + wt | | (Zalu scFv + | PKLLIYDASSLESGVPSRFSGSESGTDFTLTISSLQPEDFATYY |
| IgG1) | | arm 032- | CQQFNSYPLTFGGGTKVEIKGGGGSGGGGSGGGGSGGGGSQ |
| BGX3 | | knob) | VQLVESGGGVVQPGRSLRLSCAASGFTFSTYGMHWVRQAPG |
| | | | KGLEWVAVIWDDGSYKYYGDSVKGRFTISRDNSKNTLYLQ |
| | | | MNSLRAEDTAVYYCARDGITMVRGVMKDYFDYWGQGTLV |
| | | | TVSSGGGGSGGGGSGGGGSGGGGSEFQLQESGPGLVKPSETL |
| | | | RLTCTVSGGSISTSSYYWGWIRQPPGKGLEWIGTIYYSGTTY |
| | | | YNPSLKSRVTISVDTSKNQFSLKLKSVRAADTSIYYCARHWP |
| | | | KYNSWFDPWGQGILVTVSSASTKGPSVFPLAPSSKSTSGGTA |
| | | | ALGCLVKDYFPEPVTVSWNSGALTSGVHTFPAVLQSSGLYSL |
| | | | SSVVTVPSSSLGTQTYICNVNHKPSNTKVDKKVEPKSCDKTH |
| | | | TCPPCPAPELLGGPSVFLFPPKPKDTLMISRTPEVTCVVVDVS |
| | | | HEDPEVKFNWYVDGVEVHNAKTKPREEQYNSTYRVVSVLT |
| | | | VLHQDWLNGKEYKCKVSNKALPAPIEKTISKAKGQPREPQV |
| | | | YTLPPSREEMTKNQVSLWCLVKGFYPSDIAVEWESNGQPEN |
| | | | NYKTTPPVLDSDGSFFLYSKLTVDKSRWQQGNVFSCSVMHE |
| | | | ALHNHYTQKSLSLSPGK |
| | 172 | HC-hole | EVQLVESGGEVVQPGESLRLSCAASGFTFSSHGMHWVRQAP |
| | | (arm 109- | GKGLEWVAVISYDESDKYYADSVKGRFTISRDHSKNTLYLE |
| | | hole, HE) | MNSLRAEDTAVYYCVKDRHEGYSGWLDYWGQGTLVTVSS |
| | | | ASTKGPSVFPLAPSSKSTSGGTAALGCLVKDYFPEPVTVSWN |
| | | | SGALTSGVHTFPAVLQSSGLYSLSSVVTVPSSSLGTQTYICNV |
| | | | NHKPSNTKVDKKVEPKSCDKTHTCPPCPAPELLGGPSVFLFP |
| | | | PKPKDTLMISRTPEVTCVVVDVSHEDPEVKFNWYVDGVEVH |
| | | | NAKTKPREEQYNSTYRVVSVLTVLHQDWLNGKEYKCKVSN |
| | | | KALPAPIEKTISKAKGQPREPQVYTLPPSREEMTKNQVSLSCA |
| | | | VKGFYPSDIAVEWESNGQPENNYKTTPPVLDSDGSFFLVSKL |
| | | | TVDKSRWQQGNVFSCSVMHEALHNHYTQKSLSLSPGK |
| | 148 | LC | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | 158 | ScFv- | RASQDISSALV |
| | | LCDR1 | |
| | | (Kabat) | |
| | 159 | ScFv- | DASSLES |
| | | LCDR2 | |
| | | (Kabat) | |
| | 160 | scFv- | QQFNSYPLT |
| | | LCDR3 | |
| | | (Kabat) | |
| | 155 | ScFv- | TYGMH |
| | | HCDR1 | |
| | | (Kabat) | |
| | 156 | ScFv- | VIWDDGSYKYYGDSVKG |
| | | HCDR2 | |
| | | (Kabat) | |
| | 157 | ScFv- | DGITMVRGVMKDYFDY |
| | | HCDR3 | |
| | | (Kabat) | |
| | 56 | arm 032- | TSSYYWG |
| | | HCDR1 | |
| | 57 | arm 032- | TIYYSGTTYYNPSLKS |
| | | HCDR2 | |
| | | (Kabat) | |
| | 58 | arm 032- | HWPKYNSWFDP |
| | | HCDR3 | |
| | | (Kabat) | |
| | 6 | arm 109- | SHGMH |
| | | HCDR1 | |
| | | (Kabat) | |
| | 7 | arm 109- | VISYDESDKYYADSVKG |
| | | HCDR2 | |
| | | (Kabat) | |
| | 88 | arm 109-HE- | DRHEGYSGWLDY |
| | | HCDR3 | |
| | | (Kabat) | |
| | 66 | LCDR1 | RASQSVSSYLA |
| | | (Kabat) | |
| | 67 | LCDR2 | DASNRAT |
| | | (Kabat) | |
| | 68 | LCDR3 | QQRSNWPPT |
| | | (Kabat) | |
| | 142 | Zalu VH | QVQLVESGGGVVQPGRSLRLSCAASGFTFSTYGMHWVRQAP |
| | | | GKGLEWVAVIWDDGSYKYYGDSVKGRFTISRDNSKNTLYL |
| | | | QMNSLRAEDTAVYYCARDGITMVRGVMKDYFDYWGQGTL |
| | | | VTVSS |
| | 143 | Zalu VL | AIQLTQSPSSLSASVGDRVTITCRASQDISSALVWYQQKPGKA |
| | | | PKLLIYDASSLESGVPSRFSGSESGTDFTLTISSLQPEDFATYY |
| | | | CQQFNSYPLTFGGGTKVEIK |
| | 145 | arm 032 VH | EFQLQESGPGLVKPSETLRLTCTVSGGSISTSSYYWGWIRQPP |
| | | | GKGLEWIGTIYYSGTTYYNPSLKSRVTISVDTSKNQFSLKLKS |
| | | | VRAADTSIYYCARHWPKYNSWFDPWGQGILVTVSS |
| | 174 | arm 109- | EVQLVESGGEVVQPGESLRLSCAASGFTFSSHGMHWVRQAP |
| | | HE VH | GKGLEWVAVISYDESDKYYADSVKGRFTISRDHSKNTLYLE |
| | | | MNSLRAEDTAVYYCVKDRHEGYSGWLDYWGQGTLVTVSS |
| | 64 | VL | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPPTFGQGTKVEIK |

TABLE 31
Nucleic Acid Sequences

| | SEQ ID | | |
|---|---|---|---|
| Antibody | NO: | Description | SEQUENCE |
| T187 (NE | 175 | Zalu VH | CAAGTGCAACTGGTCGAAAGCGGCGGGGGGGTGGTGCAG |
| + wt | | | CCTGGGAGAAGCCTGAGACTGAGCTGCGCCGCTAGCGGCT |
| IgG1) | | | TCACCTTCAGCACCTACGGCATGCACTGGGTGAGACAAGC |
| BGX1 | | | CCCCGGCAAAGGCCTGGAATGGGTGGCCGTGATCTGGGAC |
| | | | GACGGCAGCTACAAGTACTACGGCGACAGCGTGAAGGGC |
| | | | AGATTCACCATCAGCAGAGACAACAGCAAGAACACCCTG |
| | | | TACCTGCAGATGAACAGCCTGAGAGCCGAGGACACCGCC |
| | | | GTGTACTACTGCGCTAGAGACGGCATCACCATGGTGAGAG |
| | | | GCGTGATGAAGGACTACTTTGACTATTGGGGGCAAGGCAC |
| | | | CCTGGTGACCGTGAGCAG |
| | 176 | Zalu VL | GCCATTCAGCTGACACAGAGCCCTAGCAGCCTGAGCGCTA |
| | | | GCGTGGGCGACAGAGTGACAATCACCTGCAGAGCTAGCC |
| | | | AAGACATCAGCAGCGCCCTGGTGTGGTATCAGCAGAAGCC |
| | | | CGGCAAGGCCCCCAAGCTGCTGATCTACGACGCTAGCAGC |
| | | | CTCGAAAGCGGCGTGCCTAGCAGATTTAGCGGCAGCGAG |
| | | | AGCGGCACCGACTTCACCCTGACCATCAGCAGCCTGCAGC |
| | | | CCGAGGACTTCGCCACCTACTACTGTCAGCAGTTCAACAG |
| | | | CTACCCCCTGACCTTCGGCGGGGGCACAAAAGTGGAAATT |
| | | | AAG |
| | 177 | arm 032 VH | GAGTTTCAGCTGCAAGAGTCCGGCCCCGGCCTGGTGAAGC |
| | | | CTAGCGAGACCCTGAGACTGACCTGCACCGTGAGCGGCGG |
| | | | CAGCATCAGCACAAGCAGCTACTACTGGGGCTGGATCAGA |
| | | | CAGCCTCCCGGCAAGGGCCTGGAGTGGATCGGCACCATCT |
| | | | ACTACAGCGGCACCACCTACTACAACCCTAGCCTGAAGAG |
| | | | CAGAGTGACCATCAGCGTGGACACAAGCAAGAATCAGTT |
| | | | CAGCCTGAAGCTGAAGAGCGTGAGAGCCGCCGACACAAG |
| | | | CATCTATTACTGCGCCCGGCACTGGCCCAAGTACAACAGC |
| | | | TGGTTCGACCCCTGGGGCCAAGGCATCCTGGTGACCGTCA |
| | | | GCAGC |
| | 178 | arm 109- | GAGGTGCAGCTGGTCGAGAGCGGGGGCGAAGTGGTCCAA |
| | | NE VH | CCCGGCGAGAGCCTGAGACTGTCCTGCGCCGCTAGCGGCT |
| | | | TCACCTTCAGCAGCCACGGCATGCACTGGGTGAGACAAGC |
| | | | CCCCGGCAAGGGCCTGGAGTGGGTGGCCGTGATCAGCTAC |
| | | | GACGAGAGCGACAAGTACTACGCCGACAGCGTGAAGGGC |
| | | | AGATTCACCATCAGCAGAGACCACAGCAAGAACACCCTGT |
| | | | ACCTGGAGATGAACAGCCTGAGAGCCGAGGACACCGCCG |
| | | | TGTACTACTGCGTGAAGGACAGAAACGAGGGCTACAGCG |
| | | | GCTGGCTGGACTACTGGGGCCAAGGCACCCTGGTGACCGT |
| | | | GTCCTCC |
| | 179 | VL | GAGATTGTGCTGACACAGAGCCCCGCCACCCTGTCCCTGT |
| | | | CCCCCGGGGAGAGAGCCACCCTGAGCTGCAGAGCTAGCC |
| | | | AAAGCGTGAGCAGCTACCTGGCCTGGTATCAGCAGAAGCC |
| | | | CGGCCAAGCCCCTAGACTGCTGATCTACGACGCTAGCAAC |
| | | | AGAGCCACCGGCATCCCCGCTAGATTCAGCGGCAGCGGCA |
| | | | GCGGCACCGACTTCACCCTGACCATCAGCAGCCTGGAGCC |
| | | | CGAGGACTTCGCCGTGTACTACTGTCAGCAGAGAAGCAAC |
| | | | TGGCCCCCCACCTTCGGCCAAGGCACCAAGGTGGAGATCA |
| | | | AG |
| T187 (NE | 175 | Zalu VH | CAAGTGCAACTGGTCGAAAGCGGGGGGGGGTGGTGCAG |
| + IgG1- | | | CCTGGGAGAAGCCTGAGACTGAGCTGCGCCGCTAGCGGCT |
| YTE) | | | TCACCTTCAGCACCTACGGCATGCACTGGGTGAGACAAGC |
| BGX5 | | | CCCCGGCAAAGGCCTGGAATGGGTGGCCGTGATCTGGGAC |
| | | | GACGGCAGCTACAAGTACTACGGCGACAGCGTGAAGGGC |
| | | | AGATTCACCATCAGCAGAGACAACAGCAAGAACACCCTG |
| | | | TACCTGCAGATGAACAGCCTGAGAGCCGAGGACACCGCC |
| | | | GTGTACTACTGCGCTAGAGACGGCATCACCATGGTGAGAG |
| | | | GCGTGATGAAGGACTACTTTGACTATTGGGGGCAAGGCAC |
| | | | CCTGGTGACCGTGAGCAG |
| | 176 | Zalu VL | GCCATTCAGCTGACACAGAGCCCTAGCAGCCTGAGCGCTA |
| | | | GCGTGGGCGACAGAGTGACAATCACCTGCAGAGCTAGCC |
| | | | AAGACATCAGCAGCGCCCTGGTGTGGTATCAGCAGAAGCC |
| | | | CGGCAAGGCCCCCAAGCTGCTGATCTACGACGCTAGCAGC |
| | | | CTCGAAAGCGGCGTGCCTAGCAGATTTAGCGGCAGCGAG |
| | | | AGCGGCACCGACTTCACCCTGACCATCAGCAGCCTGCAGC |
| | | | CCGAGGACTTCGCCACCTACTACTGTCAGCAGTTCAACAG |
| | | | CTACCCCCTGACCTTCGGCGGGGGCACAAAAGTGGAAATT |
| | | | AAG |
| | 177 | arm 032 VH | GAGTTTCAGCTGCAAGAGTCCGGCCCCGGCCTGGTGAAGC |
| | | | CTAGCGAGACCCTGAGACTGACCTGCACCGTGAGCGGCGG |
| | | | CAGCATCAGCACAAGCAGCTACTACTGGGGCTGGATCAGA |
| | | | CAGCCTCCCGGCAAGGGCCTGGAGTGGATCGGCACCATCT |
| | | | ACTACAGCGGCACCACCTACTACAACCCTAGCCTGAAGAG |
| | | | CAGAGTGACCATCAGCGTGGACACAAGCAAGAATCAGTT |
| | | | CAGCCTGAAGCTGAAGAGCGTGAGAGCCGCCGACACAAG |
| | | | CATCTATTACTGCGCCCGGCACTGGCCCAAGTACAACAGC |
| | | | TGGTTCGACCCCTGGGGCCAAGGCATCCTGGTGACCGTCA |
| | | | GCAGC |
| | 178 | arm 109- | GAGGTGCAGCTGGTCGAGAGCGGGGGCGAAGTGGTCCAA |
| | | NE VH | CCCGGCGAGAGCCTGAGACTGTCCTGCGCCGCTAGCGGCT |
| | | | TCACCTTCAGCAGCCACGGCATGCACTGGGTGAGACAAGC |
| | | | CCCCGGCAAGGGCCTGGAGTGGGTGGCCGTGATCAGCTAC |
| | | | GACGAGAGCGACAAGTACTACGCCGACAGCGTGAAGGGC |
| | | | AGATTCACCATCAGCAGAGACCACAGCAAGAACACCCTGT |
| | | | ACCTGGAGATGAACAGCCTGAGAGCCGAGGACACCGCCG |
| | | | TGTACTACTGCGTGAAGGACAGAAACGAGGGCTACAGCG |
| | | | GCTGGCTGGACTACTGGGGCCAAGGCACCCTGGTGACCGT |
| | | | GTCCTCC |
| | 179 | VL | GAGATTGTGCTGACACAGAGCCCCGCCACCCTGTCCCTGT |
| | | | CCCCCGGGGAGAGAGCCACCCTGAGCTGCAGAGCTAGCC |
| | | | AAAGCGTGAGCAGCTACCTGGCCTGGTATCAGCAGAAGCC |
| | | | CGGCCAAGCCCCTAGACTGCTGATCTACGACGCTAGCAAC |
| | | | AGAGCCACCGGCATCCCCGCTAGATTCAGCGGCAGCGGCA |
| | | | GCGGCACCGACTTCACCCTGACCATCAGCAGCCTGGAGCC |
| | | | CGAGGACTTCGCCGTGTACTACTGTCAGCAGAGAAGCAAC |
| | | | TGGCCCCCCACCTTCGGCCAAGGCACCAAGGTGGAGATCA |
| | | | AG |
| T187 (FE | 175 | Zalu VH | CAAGTGCAACTGGTCGAAAGCGGCGGGGGGGTGGTGCAG |
| + wt | | | CCTGGGAGAAGCCTGAGACTGAGCTGCGCCGCTAGCGGCT |
| IgG1) | | | TCACCTTCAGCACCTACGGCATGCACTGGGTGAGACAAGC |
| BGX2 | | | CCCCGGCAAAGGCCTGGAATGGGTGGCCGTGATCTGGGAC |
| | | | GACGGCAGCTACAAGTACTACGGCGACAGCGTGAAGGGC |
| | | | AGATTCACCATCAGCAGAGACAACAGCAAGAACACCCTG |
| | | | TACCTGCAGATGAACAGCCTGAGAGCCGAGGACACCGCC |
| | | | GTGTACTACTGCGCTAGAGACGGCATCACCATGGTGAGAG |
| | | | GCGTGATGAAGGACTACTTTGACTATTGGGGGCAAGGCAC |
| | | | CCTGGTGACCGTGAGCAG |
| | 176 | Zalu VL | GCCATTCAGCTGACACAGAGCCCTAGCAGCCTGAGCGCTA |
| | | | GCGTGGGCGACAGAGTGACAATCACCTGCAGAGCTAGCC |
| | | | AAGACATCAGCAGCGCCCTGGTGTGGTATCAGCAGAAGCC |
| | | | CGGCAAGGCCCCCAAGCTGCTGATCTACGACGCTAGCAGC |
| | | | CTCGAAAGCGGCGTGCCTAGCAGATTTAGCGGCAGCGAG |
| | | | AGCGGCACCGACTTCACCCTGACCATCAGCAGCCTGCAGC |
| | | | CCGAGGACTTCGCCACCTACTACTGTCAGCAGTTCAACAG |
| | | | CTACCCCCTGACCTTCGGCGGGGGCACAAAAGTGGAAATT |
| | | | AAG |
| | 177 | arm 032 VH | GAGTTTCAGCTGCAAGAGTCCGGCCCCGGCCTGGTGAAGC |
| | | | CTAGCGAGACCCTGAGACTGACCTGCACCGTGAGCGGCGG |
| | | | CAGCATCAGCACAAGCAGCTACTACTGGGGCTGGATCAGA |
| | | | CAGCCTCCCGGCAAGGGCCTGGAGTGGATCGGCACCATCT |
| | | | ACTACAGCGGCACCACCTACTACAACCCTAGCCTGAAGAG |
| | | | CAGAGTGACCATCAGCGTGGACACAAGCAAGAATCAGTT |
| | | | CAGCCTGAAGCTGAAGAGCGTGAGAGCCGCCGACACAAG |
| | | | CATCTATTACTGCGCCCGGCACTGGCCCAAGTACAACAGC |
| | | | TGGTTCGACCCCTGGGGCCAAGGCATCCTGGTGACCGTCA |
| | | | GCAGC |
| | 180 | arm 109- | GAGGTGCAGCTGGTCGAGAGCGGGGGCGAAGTGGTCCAA |
| | | FE VH | CCCGGCGAGAGCCTGAGACTGTCCTGCGCCGCTAGCGGCT |
| | | | TCACCTTCAGCAGCCACGGCATGCACTGGGTGAGACAAGC |
| | | | CCCCGGCAAGGGCCTGGAGTGGGTGGCCGTGATCAGCTAC |
| | | | GACGAGAGCGACAAGTACTACGCCGACAGCGTGAAGGGC |
| | | | AGATTCACCATCAGCAGAGACCACAGCAAGAACACCCTGT |
| | | | ACCTGGAGATGAACAGCCTGAGAGCCGAGGACACCGCCG |
| | | | TGTACTACTGCGTGAAGGACAGATTCGAGGGCTACAGCGG |
| | | | CTGGCTGGACTACTGGGGCCAAGGCACCCTGGTGACCGTG |
| | | | TCCTCC |
| | 179 | VL | GAGATTGTGCTGACACAGAGCCCCGCCACCCTGTCCCTGT |
| | | | CCCCCGGGGAGAGAGCCACCCTGAGCTGCAGAGCTAGCC |
| | | | AAAGCGTGAGCAGCTACCTGGCCTGGTATCAGCAGAAGCC |
| | | | CGGCCAAGCCCCTAGACTGCTGATCTACGACGCTAGCAAC |
| | | | AGAGCCACCGGCATCCCCGCTAGATTCAGCGGCAGCGGCA |
| | | | GCGGCACCGACTTCACCCTGACCATCAGCAGCCTGGAGCC |
| | | | CGAGGACTTCGCCGTGTACTACTGTCAGCAGAGAAGCAAC |
| | | | TGGCCCCCCACCTTCGGCCAAGGCACCAAGGTGGAGATCA |
| | | | AG |
| T187 (FE | 175 | Zalu VH | CAAGTGCAACTGGTCGAAAGCGGCGGGGGGGTGGTGCAG |
| + IgG1- | | | CCTGGGAGAAGCCTGAGACTGAGCTGCGCCGCTAGCGGCT |
| YTE) | | | TCACCTTCAGCACCTACGGCATGCACTGGGTGAGACAAGC |
| BGX4 | | | CCCCGGCAAAGGCCTGGAATGGGTGGCCGTGATCTGGGAC |
| | | | GACGGCAGCTACAAGTACTACGGCGACAGCGTGAAGGGC |
| | | | AGATTCACCATCAGCAGAGACAACAGCAAGAACACCCTG |
| | | | TACCTGCAGATGAACAGCCTGAGAGCCGAGGACACCGCC |
| | | | GTGTACTACTGCGCTAGAGACGGCATCACCATGGTGAGAG |
| | | | GCGTGATGAAGGACTACTTTGACTATTGGGGGCAAGGCAC |
| | | | CCTGGTGACCGTGAGCAG |
| | 176 | Zalu VL | GCCATTCAGCTGACACAGAGCCCTAGCAGCCTGAGCGCTA |
| | | | GCGTGGGCGACAGAGTGACAATCACCTGCAGAGCTAGCC |
| | | | AAGACATCAGCAGCGCCCTGGTGTGGTATCAGCAGAAGCC |
| | | | CGGCAAGGCCCCCAAGCTGCTGATCTACGACGCTAGCAGC |
| | | | CTCGAAAGCGGCGTGCCTAGCAGATTTAGCGGCAGCGAG |
| | | | AGCGGCACCGACTTCACCCTGACCATCAGCAGCCTGCAGC |
| | | | CCGAGGACTTCGCCACCTACTACTGTCAGCAGTTCAACAG |
| | | | CTACCCCCTGACCTTCGGCGGGGGCACAAAAGTGGAAATT |
| | | | AAG |
| | 177 | arm 032 VH | GAGTTTCAGCTGCAAGAGTCCGGCCCCGGCCTGGTGAAGC |
| | | | CTAGCGAGACCCTGAGACTGACCTGCACCGTGAGCGGCGG |
| | | | CAGCATCAGCACAAGCAGCTACTACTGGGGCTGGATCAGA |
| | | | CAGCCTCCCGGCAAGGGCCTGGAGTGGATCGGCACCATCT |
| | | | ACTACAGCGGCACCACCTACTACAACCCTAGCCTGAAGAG |
| | | | CAGAGTGACCATCAGCGTGGACACAAGCAAGAATCAGTT |
| | | | CAGCCTGAAGCTGAAGAGCGTGAGAGCCGCCGACACAAG |
| | | | CATCTATTACTGCGCCCGGCACTGGCCCAAGTACAACAGC |
| | | | TGGTTCGACCCCTGGGGCCAAGGCATCCTGGTGACCGTCA |
| | | | GCAGC |
| | 180 | arm 109- | GAGGTGCAGCTGGTCGAGAGCGGGGGCGAAGTGGTCCAA |
| | | FE VH | CCCGGCGAGAGCCTGAGACTGTCCTGCGCCGCTAGCGGCT |
| | | | TCACCTTCAGCAGCCACGGCATGCACTGGGTGAGACAAGC |
| | | | CCCCGGCAAGGGCCTGGAGTGGGTGGCCGTGATCAGCTAC |
| | | | GACGAGAGCGACAAGTACTACGCCGACAGCGTGAAGGGC |
| | | | AGATTCACCATCAGCAGAGACCACAGCAAGAACACCCTGT |
| | | | ACCTGGAGATGAACAGCCTGAGAGCCGAGGACACCGCCG |
| | | | TGTACTACTGCGTGAAGGACAGATTCGAGGGCTACAGCGG |
| | | | CTGGCTGGACTACTGGGGCCAAGGCACCCTGGTGACCGTG |
| | | | TCCTCC |
| | 179 | VL | GAGATTGTGCTGACACAGAGCCCCGCCACCCTGTCCCTGT |
| | | | CCCCCGGGGAGAGAGCCACCCTGAGCTGCAGAGCTAGCC |
| | | | AAAGCGTGAGCAGCTACCTGGCCTGGTATCAGCAGAAGCC |
| | | | CGGCCAAGCCCCTAGACTGCTGATCTACGACGCTAGCAAC |
| | | | AGAGCCACCGGCATCCCCGCTAGATTCAGCGGCAGCGGCA |
| | | | GCGGCACCGACTTCACCCTGACCATCAGCAGCCTGGAGCC |
| | | | CGAGGACTTCGCCGTGTACTACTGTCAGCAGAGAAGCAAC |
| | | | TGGCCCCCCACCTTCGGCCAAGGCACCAAGGTGGAGATCA |
| | | | AG |
| T187 (HE | 175 | Zalu VH | CAAGTGCAACTGGTCGAAAGCGGCGGGGGGGTGGTGCAG |
| + wt | | | CCTGGGAGAAGCCTGAGACTGAGCTGCGCCGCTAGCGGCT |
| IgG1) | | | TCACCTTCAGCACCTACGGCATGCACTGGGTGAGACAAGC |
| BGX3 | | | CCCCGGCAAAGGCCTGGAATGGGTGGCCGTGATCTGGGAC |
| | | | GACGGCAGCTACAAGTACTACGGCGACAGCGTGAAGGGC |
| | | | AGATTCACCATCAGCAGAGACAACAGCAAGAACACCCTG |
| | | | TACCTGCAGATGAACAGCCTGAGAGCCGAGGACACCGCC |
| | | | GTGTACTACTGCGCTAGAGACGGCATCACCATGGTGAGAG |
| | | | GCGTGATGAAGGACTACTTTGACTATTGGGGGCAAGGCAC |
| | | | CCTGGTGACCGTGAGCAG |
| | 176 | Zalu VL | GCCATTCAGCTGACACAGAGCCCTAGCAGCCTGAGCGCTA |
| | | | GCGTGGGCGACAGAGTGACAATCACCTGCAGAGCTAGCC |
| | | | AAGACATCAGCAGCGCCCTGGTGTGGTATCAGCAGAAGCC |
| | | | CGGCAAGGCCCCCAAGCTGCTGATCTACGACGCTAGCAGC |
| | | | CTCGAAAGCGGCGTGCCTAGCAGATTTAGCGGCAGCGAG |
| | | | AGCGGCACCGACTTCACCCTGACCATCAGCAGCCTGCAGC |
| | | | CCGAGGACTTCGCCACCTACTACTGTCAGCAGTTCAACAG |
| | | | CTACCCCCTGACCTTCGGCGGGGGCACAAAAGTGGAAATT |
| | | | AAG |
| | 177 | arm 032 VH | GAGTTTCAGCTGCAAGAGTCCGGCCCCGGCCTGGTGAAGC |
| | | | CTAGCGAGACCCTGAGACTGACCTGCACCGTGAGCGGCGG |
| | | | CAGCATCAGCACAAGCAGCTACTACTGGGGCTGGATCAGA |
| | | | CAGCCTCCCGGCAAGGGCCTGGAGTGGATCGGCACCATCT |
| | | | ACTACAGCGGCACCACCTACTACAACCCTAGCCTGAAGAG |
| | | | CAGAGTGACCATCAGCGTGGACACAAGCAAGAATCAGTT |
| | | | CAGCCTGAAGCTGAAGAGCGTGAGAGCCGCCGACACAAG |
| | | | CATCTATTACTGCGCCCGGCACTGGCCCAAGTACAACAGC |
| | | | TGGTTCGACCCCTGGGGCCAAGGCATCCTGGTGACCGTCA |
| | | | GCAGC |
| | 181 | arm 109- | GAGGTGCAGCTGGTCGAGAGCGGGGGCGAAGTGGTCCAA |
| | | HE VH | CCCGGCGAGAGCCTGAGACTGTCCTGCGCCGCTAGCGGCT |
| | | | TCACCTTCAGCAGCCACGGCATGCACTGGGTGAGACAAGC |
| | | | CCCCGGCAAGGGCCTGGAGTGGGTGGCCGTGATCAGCTAC |
| | | | GACGAGAGCGACAAGTACTACGCCGACAGCGTGAAGGGC |
| | | | AGATTCACCATCAGCAGAGACCACAGCAAGAACACCCTGT |
| | | | ACCTGGAGATGAACAGCCTGAGAGCCGAGGACACCGCCG |
| | | | TGTACTACTGCGTGAAGGACAGACACGAGGGCTACAGCG |
| | | | GCTGGCTGGACTACTGGGGCCAAGGCACCCTGGTGACCGT |
| | | | GTCCTCC |
| | 179 | VL | GAGATTGTGCTGACACAGAGCCCCGCCACCCTGTCCCTGT |
| | | | CCCCCGGGGAGAGAGCCACCCTGAGCTGCAGAGCTAGCC |
| | | | AAAGCGTGAGCAGCTACCTGGCCTGGTATCAGCAGAAGCC |
| | | | CGGCCAAGCCCCTAGACTGCTGATCTACGACGCTAGCAAC |
| | | | AGAGCCACCGGCATCCCCGCTAGATTCAGCGGCAGCGGCA |
| | | | GCGGCACCGACTTCACCCTGACCATCAGCAGCCTGGAGCC |
| | | | CGAGGACTTCGCCGTGTACTACTGTCAGCAGAGAAGCAAC |
| | | | TGGCCCCCCACCTTCGGCCAAGGCACCAAGGTGGAGATCA |
| | | | AG |

### Example 24: Synthesis of Cytotoxic Agents (Payloads)

### UPLC Analysis Methods

Method A: Mobile phase A: 0.1% FA in water, B: MeCN; Gradient: 10% B maintain 0.2 min, 10%-95% B, 5.8 min 95% B maintain 0.5 min; Flow rate: 0.6 mL/min; Column: ACQUITY UPLC® BEH C18 1.7 μm.

Method B: Mobile phase A: 0.1% FA in water, B: MeCN; Gradient: 10% B maintain 0.5 min, 10%-90% B, 2.5 min 90% B maintain 0.2 min; Flow rate: 0.6 mL/min; Column: ACQUITY UPLC® BEH C18 1.7 μm.

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

Payload P-A (CAS No.: 2495742-21-5) (Table 32) was purchased from ShangHai HaoYuan MedChemExpress Co. Ltd.

### Payload P-1

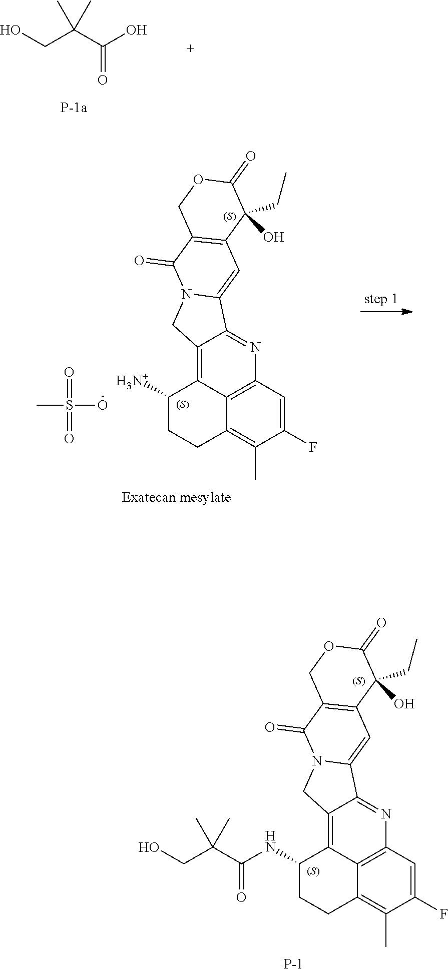

### Step 1: N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (P-1)

To a mixture of P-1a (5 mg, 0.042 mmol) and HATU (16 mg, 0.042 mmol) in DMF (1 mL) were added DIPEA (21 μL, 16 mg, 0.13 mmol) and exatecan mesylate (purchased from ShangHai HaoYuan MedChemExpress CO. LTD, 23 mg, 0.043 mmol). The resulting brown mixture was stirred at r.t. for 2 h. On completion of the reaction, the mixture was purified by prep-HPLC (TFA) (Method: column: XBridge Prep C18 OBD 5 um 19*150 mm; Mobile phase: A-water (0.1% TFA): B-acetonitrile; Flow rate: 20 mL/min). The fraction was lyophilized to give P-1 (15 mg, 65.5% yield) as a white powder.

¹H NMR (400 MHz, DMSO-d₆): δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J=7.2 Hz, 3H). MS (ESI) m/z: 536.4 [M+H]⁺

TABLE 32
Payload List

| Payload | |
|---|---|
| ID | Payload Structure |
| P-A | 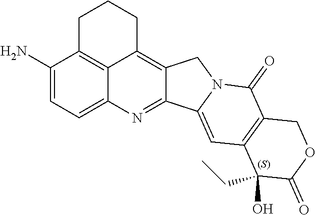 |
| P-1 | 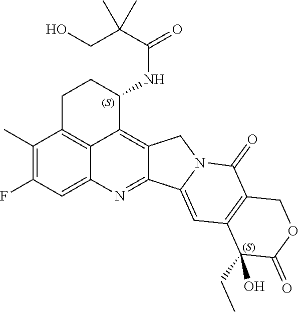 |

### Example 25: Synthesis of Linker-Payloads (“LD”)

Linker-payload LD-1-A (SG-3932; CAS No.: 2495742-34-0) (Table 33) was purchased from ShangHai HaoYuan MedChemExpress Co. Ltd.

### Linker-Payload LD-2-1

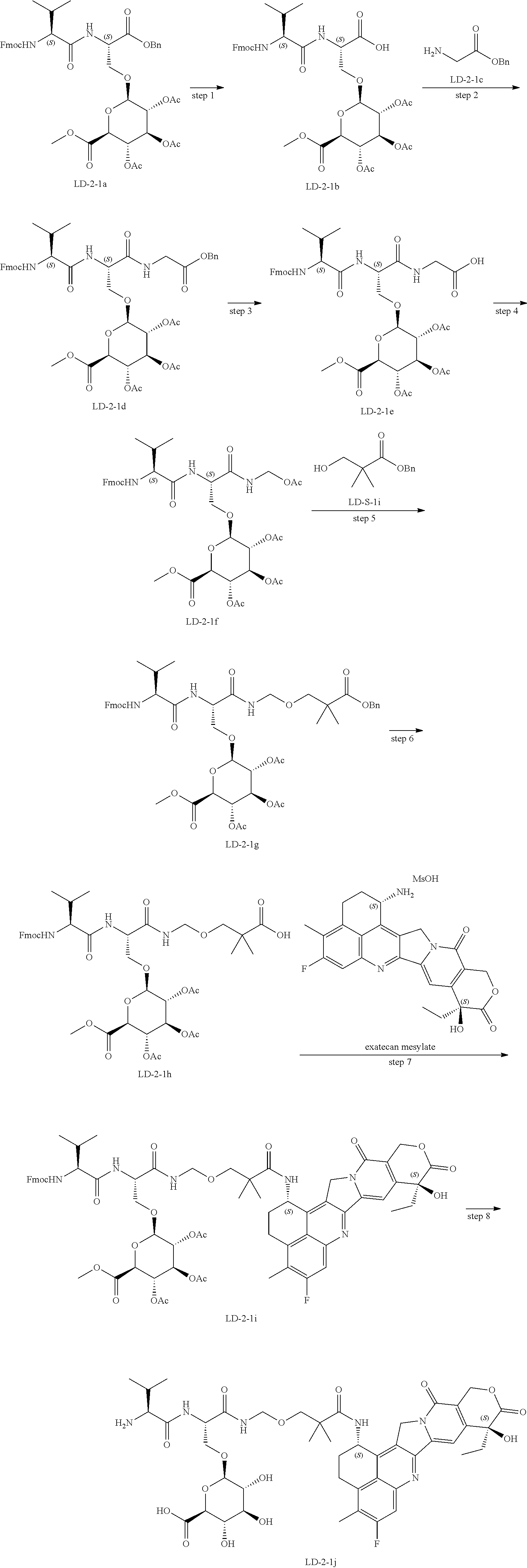

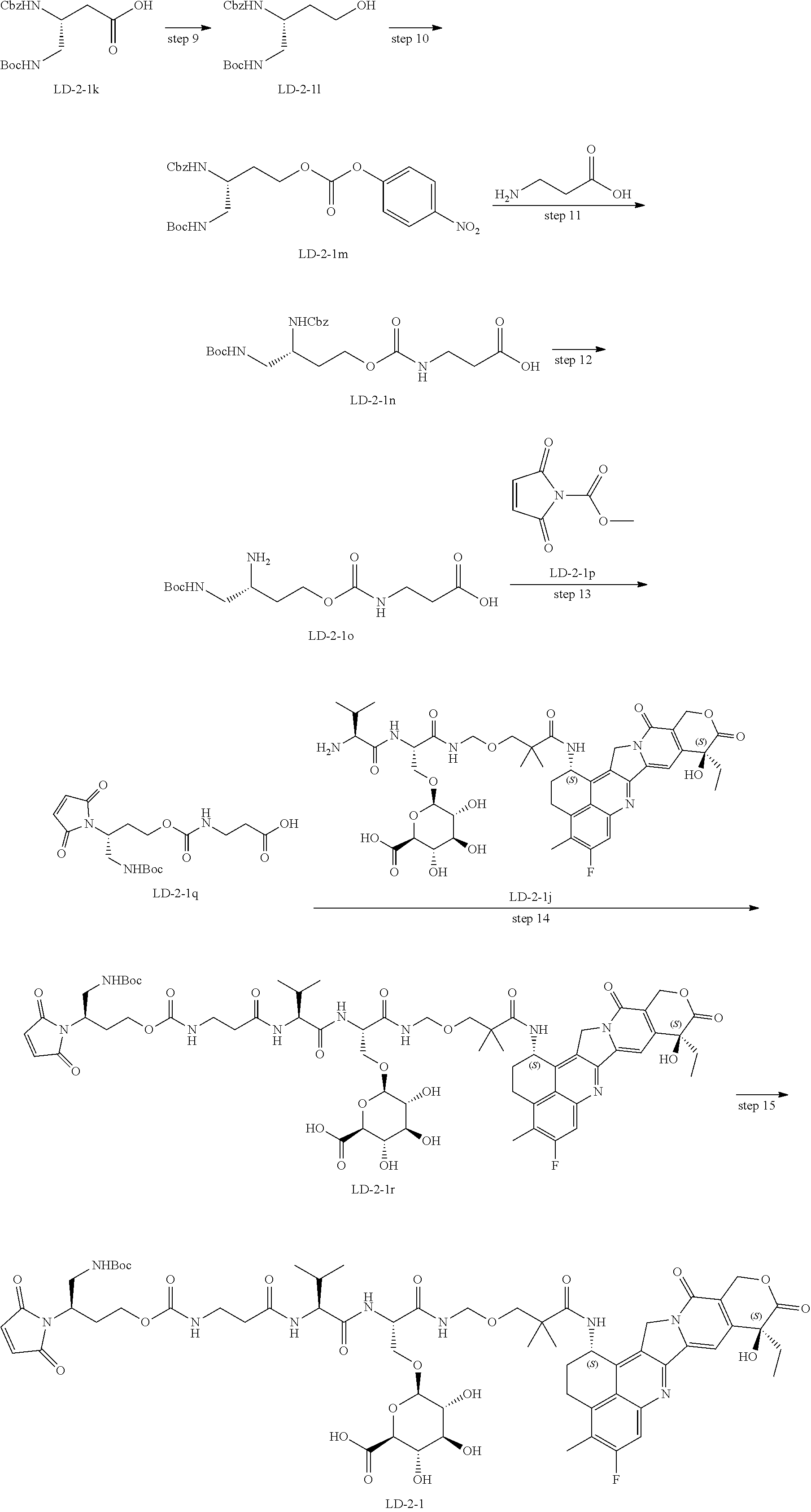

### Step 1: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (LD-2-1b)

To a mixture of LD-2-1a (4.1 g, 4.92 mmol, purchased from MCE) in MeOH (50 mL), THE (10 mL), and DCM (20 mL) was added wet Pd/C (400 mg, 10% purity). The black suspension was purged with H₂ balloon three times and then stirred at r.t. for 1 hr. The black suspension was filtered through a pad of celite and washed with MeOH (200 mL). Organic layers were combined and concentrated under vacuum to give LD-2-1b (3650 mg, 99.8% yield) as an off-white solid.

MS (ESI) m/z: 743.6 [M+H]⁺

### Step 2: (2R,3R,4S,5S,6S)-2-((S)-2-((S)-2-((((9H-fluoren-9-yl)methoxy)carbonyl)amino)-3-methylbutanamido)-3-((2-(benzyloxy)-2-oxoethyl)amino)-3-oxopropoxy)-6-(methoxycarbonyl)tetrahydro-2H-pyran-3,4,5-triyl triacetate (LD-2-1d)

To a solution of LD-2-1b (3.65 g, 4.92 mmol) and LD-2-1c (1.66 g, 4.92 mmol) in DMF (50 mL) were added HATU (1.87 g, 4.92 mmol) and DIPEA (1.59 g, 12.29 mmol). The mixture was stirred at r.t. for 30 min. The mixture was purified by flash column chromatography (MeOH/DCM=0-10%), and the fraction was concentrated under vacuum to give LD-2-1d (3.8 g, 86.9% yield) as an off-white foamed solid.

MS (ESI) m/z: 890.7 [M+H]⁺

### Step 3: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serylglycine (LD-2-1e)

To a mixture of LD-2-1d (3.8 g, 4.27 mmol) in MeOH (150 mL) and DCM (50 mL) was added wet Pd/C (400 mg, 10% purity). The black suspension was purged with H₂ balloon three times and then stirred at r.t. for 40 min. The black suspension was filtered through a pad of celite, and washed with MeOH (150 mL). Organic layers were combined and concentrated under vacuum to give LD-2-1e (3.3 g, 96.6% yield) as an off-white solid.

MS (ESI) m/z: 800.7 [M+H]⁺

### Step 4: (2R,3R,4S,5S,6S)-2-((S)-2-((S)-2-((((9H-fluoren-9-yl)methoxy)carbonyl)amino)-3-methylbutanamido)-3-((acetoxymethyl)amino)-3-oxopropoxy)-6-(methoxycarbonyl)tetrahydro-2H-pyran-3,4,5-triyl triacetate (LD-2-1f)

To a solution of LD-2-1e (3.3 g, 4.13 mmol) in DMF (30 mL) were added Pb(OAc)₄ (2.74 g, 6.19 mmol), Cu(OAc)₂ (74.9 mg, 0.41 mmol), and HOAc (247.8 mg, 4.13 mmol). The resulting dark-colored mixture was purged with N₂ balloon three times and then stirred at 65° C. for 40 min until the mixture turned deep blue. The mixture was diluted with EtOAc (300 mL), washed with brine (100 mL*3), dried over Na₂SO₄, filtered, and concentrated under vacuum to give a residue. It was purified by flash column chromatography (MeOH/DCM=0/100-10/90), and the fraction was concentrated under vacuum to give LD-2-1f (2.8 g, 83.4% yield) as a pale-yellow solid.

MS (ESI) m/z: 836.6 [M+Na]⁺

### Step 5: (2R,3R,4S,5S,6S)-2-((S)-2-((S)-2-((((9H-fluoren-9-yl)methoxy)carbonyl)amino)-3-methylbutanamido)-3-(((3-(benzyloxy)-2,2-dimethyl-3-oxopropoxy)methyl)amino)-3-oxopropoxy)-6-(methoxycarbonyl)tetrahydro-2H-pyran-3,4,5-triyl triacetate (LD-2-1g)

A white suspension mixture of LD-2-1f (300 mg, 0.37 mmol), LD-1-1i (154 mg, 0.74 mmol) and 4 Å molecular sieve (200 mg) in anhydro THE (10 mL) was stirred at r.t. for 10 min. Sc(OTf)₃ (217.9 mg, 0.44 mmol) was added and the resulting yellow suspension was stirred at r.t. for 4 hr. The yellow suspension mixture was filtered through a pad of celite and washed with EtOAc (30 mL). Combined organic layers were washed with sat.NaHCO₃ (30 mL) and brine (30 mL), dried over Na₂SO₄, filtered and the filtrate was concentrated under vacuum to give a residue. It was purified by silica gel column (MeOH/DCM=0/100-5/95), and the fraction was concentrated under vacuum to give LD-2-1g (275 mg, 77.5% yield) as a white foam solid.

MS (ESI) m/z: 984.8 [M+Na]⁺

### Step 6: (5S,8S)-1-(9H-fluoren-9-yl)-5-isopropyl-14,14-dimethyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazapentadecan-15-oic acid (LD-2-1h)

To a solution of LD-2-1g (275 mg, 0.29 mmol) in MeOH (10 mL) was added wet Pd/C (55 mg, 10% purity). The black suspension was purged with H₂ balloon three times then stirred at r.t. for 2 hr. The mixture was filtered through a syringe head and washed with MeOH (15 mL), and concentrated under vacuum to give LD-2-1h (230 mg, crude) as a white foam solid.

MS (ESI) m/z: 894.6 [M+Na]⁺

### Step 7: (2R,3R,4S,5S,6S)-2-((S)-2-((S)-2-((((9H-fluoren-9-yl)methoxy)carbonyl)amino)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl triacetate (LD-2-1i)

To a mixture of LD-2-1h (230 mg, crude), exatecan mesylate (140 mg, 0.26 mmol), and HATU (100.3 mg, 0.26 mmol) in DMF (5 mL) was added DIPEA (102 mg, 0.79 mmol). The resulting brown mixture was stirred at r.t. for 1 hr. The mixture was diluted with EtOAc (20 mL), washed with brine (20 mL*3), dried over Na2SO4, filtered, and concentrated under vacuum to give a residue. It was purified by flash column chromatography (MeOH/DCM=0%-3%), and concentrated under vacuum to give LD-2-1i (325 mg, 95.6% yield) as an off-white foam solid.

MS (ESI) m/z: 1289.9 [M+H]⁺

### Step 8: (2S,3S,4S,5R,6R)-6-((S)-2-((S)-2-amino-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (LD-2-1j)

To a solution of LD-2-1i (325 mg, 0.25 mmol) in DMF (5 mL) was added Et₂NH (523.2 mg, 5.06 mmol). The mixture was stirred at r.t. for 20 min. then concentrated under vacuum to give a crude product. The crude product was dissolved in MeOH (6 mL), K₂CO₃ (174.7 mg, 1.26 mmol) was added and stirred at r.t. for 10 min. Then H₂O (2 mL) was added to the mixture and stirred at r.t. for 30 min. The mixture was acidified with sat.KHSO₄ at 0° C. to pH=3. The mixture was filtered and purified by prep-HPLC (0.1% FA), and the fraction was lyophilized to give LD-2-1j (140 mg, 59.7% yield) as a pale yellow solid.

MS (ESI) m/z: 927.4 [M+H]⁺

¹H NMR (400 MHz, DMSO) δ 9.56 (s, 1H), 8.39 (s, 1H), 8.07 (d, J=8.4 Hz, 1H), 7.77 (d, J=11.2 Hz, 1H), 7.31 (s, 1H), 6.52 (s, 1H), 5.54 (dd, J=13.2, 7.2 Hz, 1H), 5.43 (s, 2H), 5.18 (dd, J=41.6, 18.8 Hz, 2H), 5.09-5.02 (m, 1H), 4.96 (s, 1H), 4.62 (dd, J=10.0, 6.8 Hz, 1H), 4.56-4.44 (m, 2H), 4.19 (d, J=7.6 Hz, 1H), 3.82 (dd, J=10.8, 6.8 Hz, 1H), 3.61 (dd, J=11.6, 6.4 Hz, 2H), 3.17-3.05 (m, 4H), 2.94 (t, J=8.0 Hz, 1H), 2.39 (s, 3H), 2.11 (dt, J=21.3, 7.6 Hz, 2H), 2.03-1.93 (m, 2H), 1.92-1.78 (m, 3H), 1.12 (d, J=8.0 Hz, 6H), 0.87 (dd, J=13.0, 6.6 Hz, 9H).

### Step 9: benzyl tert-butyl (4-hydroxybutane-1,2-diyl)(R)-dicarbamate (LD-2-1l)

LD-2-1k (2.00 g, 5.68 mmol) and K₂CO₃ (863 mg, 6.24 mmol) were added into DMF (10 mL) followed by the dropwise addition of CH₃I (1.61 g, 11.35 mmol) at 0° C. The resulting mixture was stirred at 0° C. for 20 min and allowed to warm to 25° C. and further stirred at 25° C. for 60 min. The reaction process was monitored by TLC (petroleum ether/EtOAc) and LCMS. After complete reaction, the reaction mixture was diluted with EtOAc (80 mL) and washed with brine (30 mL*3) and H₂O (30 mL*2). The organic layer was dried over anhydrous Na₂SO₄, filtered, and concentrated under reduced pressure to afford the methyl ester (2.08 g, quant.) as a light-yellow solid.

The methyl ester (1.00 g, 2.73 mmol) was dissolved in MeOH (15 mL) followed by the addition of LiBH₄ (2 M stock solution in THF, 6.80 mL) at 0° C. The resulting mixture was stirred at 25° C. for 2 h. Reaction process was monitored by LCMS and TLC. After complete reaction, saturated aqueous NH₄Cl (10 mL) was added to quench the reaction. The reaction mixture was diluted with H₂O (80 mL) and extracted with EtOAc (50 mL*3). The combined organic layers were washed with brine (40 mL*2) and water (40 mL*2), dried over anhydrous Na₂SO₄, filtered, and concentrated under reduced pressure and further purified by flash column chromatography (petroleum ether/EtOAc) to afford LD-2-1l (760 mg, 82.3% yield) as a white solid.

MS (ESI) m/z: 239.2 [M-Boc+H]⁺

### Step 10: benzyl tert-butyl (4-(((4-nitrophenoxy)carbonyl)oxy)butane-1,2-diyl)(R)-dicarbamate (LD-2-1m)

LD-2-1l (300 mg, 0.89 mmol) and bis(4-nitrophenyl) carbonate (405 mg, 1.33 mmol) were dissolved in DMF (5 mL) followed by the addition of DIEA (229 mg, 1.77 mmol). The resulting mixture was stirred at 25° C. for 3 h. After complete reaction, the reaction mixture was diluted with EtOAc (100 mL) and washed with brine (35 mL*3) and water (35 mL*3). The organic layer was dried over anhydrous Na₂SO₄, filtered, concentrated under reduced pressure, and further purified by flash column chromatography (petroleum ether/EtOAc) to afford LD-2-1m as a white solid (371 mg, 83.1% yield).

MS (ESI) m/z: 404.4 [M-Boc+H]⁺

### Step 11: (R)-7-(((benzyloxy)carbonyl)amino)-2,2-dimethyl-4,11-dioxo-3,10-dioxa-5,12-diazapentadecan-15-oic acid (LD-2-1n)

LD-2-1m (420 mg, 0.83 mmol) and 3-aminopropanoic acid (149 mg, 1.67 mmol) were dissolved in DMF (5 mL) followed by the addition of aqueous NaHCO₃ (1M, 5 mL). The resulting mixture was stirred at 25° C. for 2 h. Reaction was monitored by LCMS. After complete reaction, the reaction mixture was concentrated and purified by flash column chromatography (DCM:MeOH) to afford LD-2-1n as a pale-yellow solid (339 mg, 89.6% yield).

MS (ESI) m/z: 354.4 [M-Boc+H]⁺

### Step 12: (R)-7-amino-2,2-dimethyl-4,11-dioxo-3,10-dioxa-5,12-diazapentadecan-15-oic acid (LD-2-1o)

LD-2-1n (360 mg, 0.79 mmol) was dissolved in MeOH (18 mL) followed by the addition of Pd/C (wet base, 108 mg) and NH₃-MeOH (7 M, 2 drops). The resulting mixture was stirred at r.t. under H₂ (15 psi) for 2.5 h. After complete reaction, the reaction mixture was filtered and concentrated under reduced pressure to afford LD-2-1o as a clear syrup (252 mg, quant.). The crude product was used directly in the next step without purification.

MS (ESI) m/z: 320.3 [M+H]⁺

### Step 13: (R)-7-(2,5-dioxo-2,5-dihydro-1H-pyrrol-1-yl)-2,2-dimethyl-4,11-dioxo-3,10-dioxa-5,12-diazapentadecan-15-oic acid (LD-2-1q)

LD-2-1o (250 mg, 0.78 mmol) and LD-2-1p (243 mg, 1.57 mmol) were dissolved in a mixed solvent of ACN (8 mL) and aqueous NaHCO₃ (1 M, 16 mL) at 0° C. The resulting mixture was stirred at 0° C. for 0.5 h and further stirred at 25° C. until the reaction was complete. Then the reaction mixture was acidified with aq. KHSO₄ (1M) to pH˜3 and extracted with EtOAc (40 mL*3). The combined organic layers were dried over anhydrous Na₂SO₄, filtered, and concentrated under reduced pressure to afford a yellow oil as the crude product, which was purified by flash column chromatography (DCM:MeOH) to afford LD-2-1q (280 mg, 89.6% yield) as a white solid.

MS (ESI) m/z: 422.3 [M+Na]⁺

### Step 14: (2S,3S,4S,5R,6R)-6-(((7R,17S,20S)-7-(2,5-dioxo-2,5-dihydro-1H-pyrrol-1-yl)-20-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-17-isopropyl-2,2-dimethyl-4,11,15,18-tetraoxo-3,10-dioxa-5,12,16,19-tetraazahenicosan-21-yl)oxy)-3,4,5-trihydroxytetrahydro-2H-pyran-2-carboxylic acid (LD-2-1r)

To a solution of compound LD-2-1q (230 mg, 0.58 mmol) in DMF (6 mL) were added HATU (200 mg, 0.53 mmol) and DIEA (186 mg, 1.43 mmol). The mixture was stirred at r.t. for 10 min. Compound LD-2-1j (500 mg, 0.48 mmol) was added into the mixture and stirred at r.t. for 1 h. The reaction mixture was poured into MTBE (50 ml) and i-PrOH (5 mL), stirred for 10 min, filtered and dried. The crude product LD-2-1r (628 mg, theoretical yield) was used in the next step without further purification.

MS (ESI) m/z: 1331.0 [M+Na]⁺

### Step 15: (2S,3S,4S,5R,6R)-6-(((2S,5S,15R)-16-amino-15-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-5-isopropyl-4,7,11-trioxo-12-oxa-3,6,10-triazahexadecyl)oxy)-3,4,5-trihydroxytetrahydro-2H-pyran-2-carboxylic acid (LD-2-1)

To a solution of compound LD-2-1r (628 mg, 0.48 mmol) in DCM (10 mL) were added ZnBr₂ (5.4 g, 24 mmol). The mixture was stirred at 39-40° C. for 16 h. The mixture was concentrated and dissolved in H₂O (0.1% FA, 9 mL). The solution was purified by prep-HPLC (Method: column: XBridge Prep C18 OBD 5 μm 19*150 mm; Mobile phase: A-water (0.1% TFA): B-acetonitrile; Flow rate: 20 mL/min) to give compound LD-2-1 (TFA salt, 240 mg, 38.2% yield for 2 step) as a light-yellow solid.

MS (ESI) m/z: 1208.9 [M+H]⁺

### Linker-Payload LD-3-1

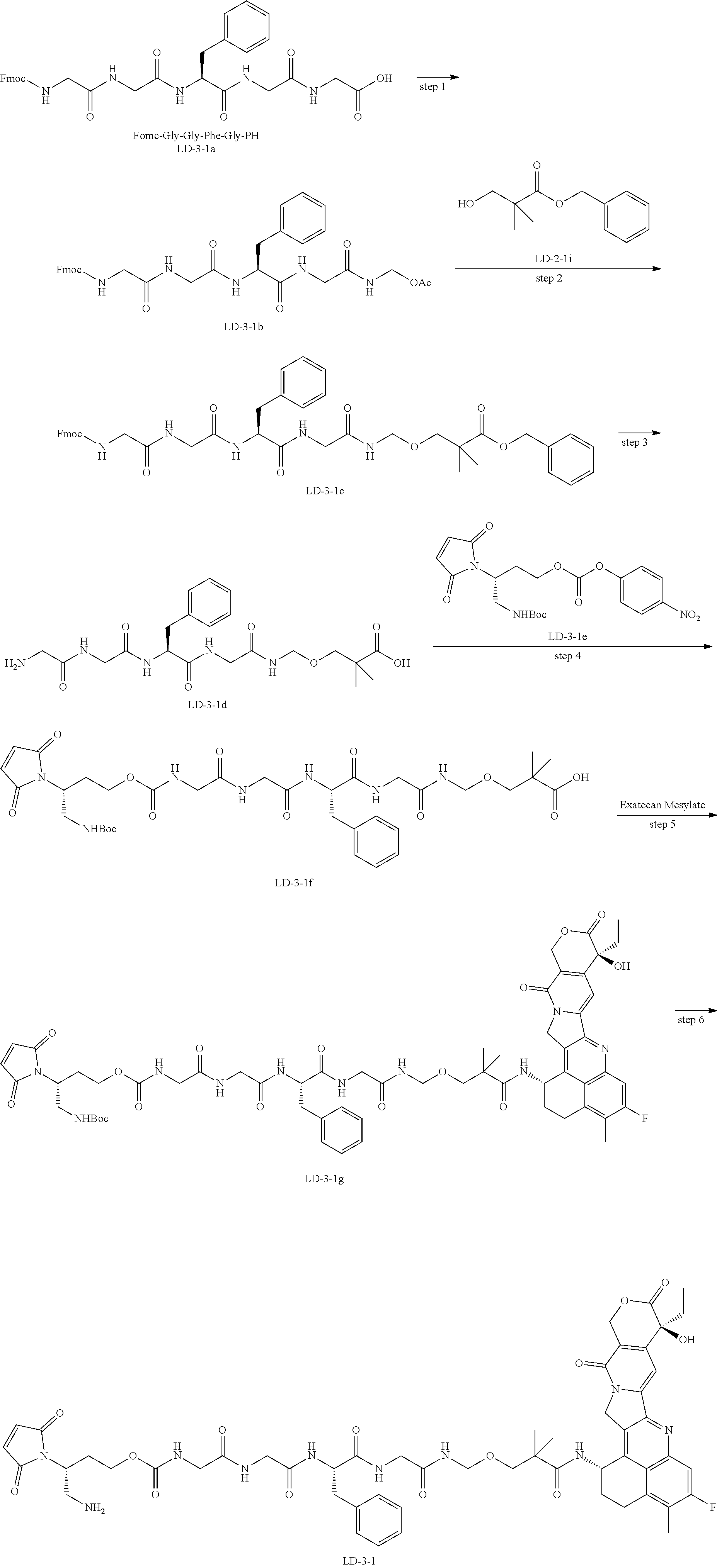

### Step 1: (S)-11-benzyl-1-(9H-fluoren-9-yl)-3,6,9,12,15-pentaoxo-2-oxa-4,7,10,13,16-pentaazaheptadecan-17-yl acetate (LD-3-1b)

LD-3-1a (30 g, 48.7 mmol, purchased from MedChemExpress) was dissolved in DMF (300 mL) followed by the addition of Pb(OAc)₄ (27.7 g, 73.1 mmol), Cu(OAc)₂ (4.86 g, 24.4 mmol) and HOAc (7.32 g, 36.3 mmol) at 25° C. After addition, the resulting mixture was stirred at 70° C. for 1 h under nitrogen atmosphere. LC-MS indicated the reaction was completed. After complete reaction, the reaction mixture was diluted with EtOAc (200 mL*3) and washed with brine (200 mL*3) and H₂O (100 mL*2). The organic phase was dried with anhydrous Na₂SO₄, filtrated, and concentrated under vacuum. The mixture was triturated with MTBE (200 mL) to obtain LD-3-1b (30 g, 92.9% yield) as a yellow solid.

MS (ESI) m/z: 652.8 [M+Na⁺]

### Step 2: benzyl (S)-11-benzyl-1-(9H-fluoren-9-yl)-20,20-dimethyl-3,6,9,12,15-pentaoxo-2,18-dioxa-4,7,10,13,16-pentaazahenicosan-21-oate (LD-3-1c)

LD-3-1b (10.1 g, 16.04 mmol), LD-2-1i (6.7 g, 32.08 mmol), and 4 Å molecular sieves (2.5 g) were weighed in an oven-dried flask and mixed with THE (130 mL). The mixture was stirred at room temperature for 30 min. Sc(OTf)₃ (11.8 g, 24.06 mmol) was added to the mixture, and then stirred for 1 h at 55° C. under nitrogen atmosphere. The reaction mixture was filtered through a pad of celite washed with EtOAc (200 mL) and quenched with sat. NaHCO₃ (100 mL). The aqueous layer was extracted with EtOAc (100 mL*2). Combined organic layers were washed with brine (200 mL), and dried over Na₂SO₄, filtered, and the filtrate was concentrated under reduced pressure to obtain the crude product, which was further purified by silica gel column chromatography (80 g, DCM/MeOH=0%-30%) to give LD-3-1c (12.2 g, 90% purity) as a white solid.

MS (ESI) m/z: 778.6 [M+H]⁺

### Step 3: (S)-1-amino-7-benzyl-16,16-dimethyl-2,5,8,11-tetraoxo-14-oxa-3,6,9,12-tetraazaheptadecan-17-oic acid (LD-3-1d)

To a solution of LD-3-1c (12.2 g, 15.69 mmol) in DMF (60 mL) was added DBU (1.64 mL, 10.98 mmol) in an ice-bath. The mixture was stirred at the same temperature for 30 min. The mixture was diluted with H₂O (60 mL), and extracted with MTBE (60 mL*3). To the aqueous phase was added wet Pd/C (2.0 mg, 10%) then purged with H₂ balloon three times, then stirred at r.t. overnight. Next it was filtered through a pad of celite and washed with H₂O (50 mL). The filtration was extracted with 2-methyl-THF (50 mL*3), then the aqueous phase was concentrated under high vacuum to give a yellow oil. The crude product was triturated with acetonitrile for 1 hr at r.t., filtered, and the cake was washed with acetonitrile (30 mL) and dried under vacuum to give LD-3-1d (3.0 g, pure) as a white solid.

MS (ESI) m/z: 466.5 [M+H]⁺

### Step 4: (7R,19S)-19-benzyl-7-(2,5-dioxo-2,5-dihydro-1H-pyrrol-1-yl)-2,2,28,28-tetramethyl-4,11,14,17,20,23-hexaoxo-3,10,26-trioxa-5,12,15,18,21,24-hexaazanonacosan-29-oic acid (LD-3-1f)

To a mixture of LD-3-1d (2.1 g, 4.5 mmol) and LD-3-1e (2.03 g, 4.5 mmol, synthesized according to a modified synthetic procedure from Tetrahedron Letters 54 (2013) 349-3495)) was added DIPEA (0.82 mL, 4.74 mmol). The resulting light-yellow mixture was stirred at r.t. for 1.5 hr. The mixture was concentrated under vacuum and purified by silica gel column chromatography (20 g, DCM/MeOH) to give LD-3-1f (2.66 g, 85% purity) as a white solid.

MS (ESI) m/z: 776.6 [M+H]⁺

### Step 5: (R)-4-((tert-butoxycarbonyl)amino)-3-(2,5-dioxo-2,5-dihydro-1H-pyrrol-1-yl)butyl ((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)carbamate (LD-3-1g)

To a mixture of LD-3-1f (2.66 g, 3.4 mmol), exatecan mesylate (purchased from MCE, 1.8 g, 3.43 mmol) and HATU (1.43 g, 4.5 mmol) in DMF (30 mL) was added DIPEA (1.2 mL, 6.8 mmol). The mixture was stirred at r.t. for 1 hr. The reaction solution was added dropwise to the MTBE solution. The formed precipitate was collected and centrifuged to obtain a brown oil, which was purified by reverse phase column (C18, 80 g, acetonitrile/H₂O (0.1% FA)). The fraction was concentrated under vacuum to give LD-3-1g (2.25 g, 97% purity) as an off-white solid.

MS (ESI) m/z: 1194.9 [M+H]⁺

### Step 6: (R)-4-amino-3-(2,5-dioxo-2,5-dihydro-1H-pyrrol-1-yl)butyl ((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)carbamate Trifluoroacetate (LD-3-1)

To a mixture of LD-3-1g (150 mg, 0.126 mmol) in DCM (3 mL) was added ZnBr₂ (1.13 g, 5.03 mmol). The resulting yellow suspension was stirred for 60 hr. The solvent was then removed by vacuum and the mixture was re-dissolved in acetonitrile/H₂O (0.1% TFA), and purified by prep-HPLC (0.1% TFA) (Method: column: XBridge Prep C18 OBD 5 μm 19*150 mm; Mobile phase: A-water (0.1% TFA): B-acetonitrile; Flow rate: 20 mL/min, gradient: B/A=10%-35%-36%-95%). The fraction was lyophilized to give LD-3-1 (85 mg, 99% purity) as a light-yellow powder.

MS (ESI) m/z: 1093.8 [M+H]⁺

### Linker-Payload LD-4-1

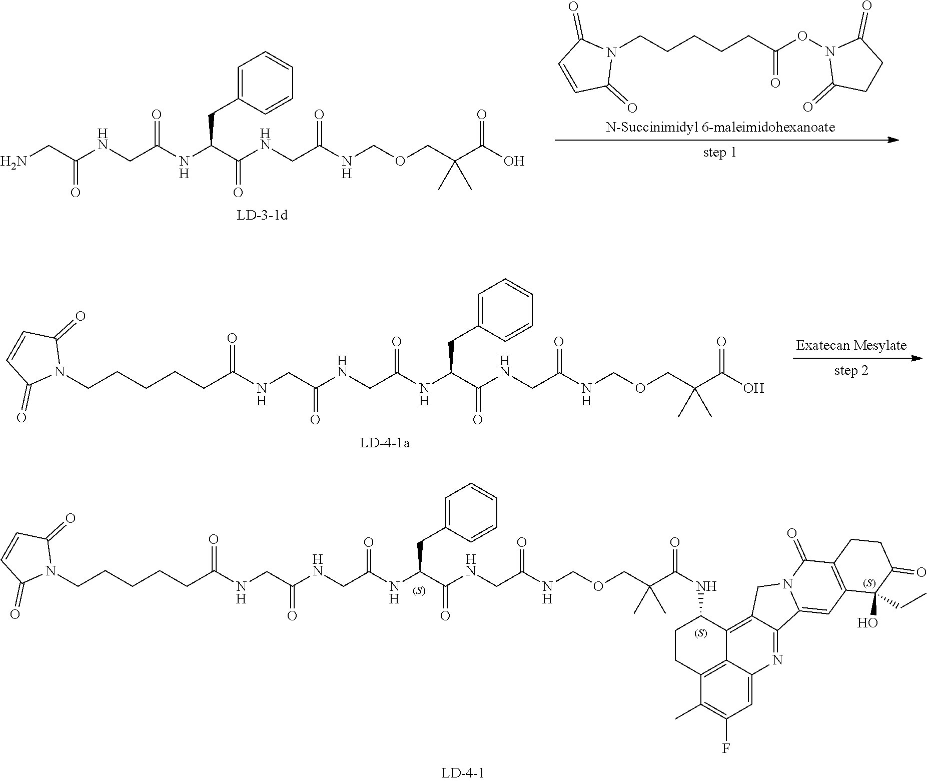

### Step 1: 3-({2-[(2S)-2-(2-{2-[16-(2,5-dioxo-2,5-dihydro-1H-pyrrol-1-yl)hexanamido]acetamido}acetamido)-3-phenylpropanamido]acetamido}methoxy)-2,2-dimethylpropanoic acid (LD-4-1a)

To a suspension of LD-3-1d (1.80 g, 3.87 mmol) and TEA (1.16 g, 11.6 mmol) in DMF (25 mL) was added N-succinimidyl 6-maleimidohexanoate (1.31 g, 4.25 mmol) at 25° C. The reaction solution was stirred at 25° C. for 3 hrs. After the reaction completed, the mixture was concentrated and the residue was purified by silica gel column chromatography (DCM/MeOH=95/5) to provide LD-4-1a (1.8 g, 70% yield) as a white solid.

MS (ESI) m/z: 659.7 [1M+H]⁺

Step 2: N—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-6-(2,5-dioxo-2,5-dihydro-1H-pyrrol-1-yl)hexanamide (LD-4-1)

To a mixture of LD-4-1a (300 mg, 0.46 mmol) and HATU (225 mg, 0.59 mmol) in DMF (3.0 mL) was added exatecan mesylate (293 mg, 0.55 mmol) and DIPEA (313 μL, 1.8 mmol) at 0° C. The mixture was stirred at 0° C. for 20 mins. The mixture was quenched with AcOH (80 μL, 1.35 mmol) and purified by prep-HPLC (Method: column XBridge Prep C18 OBD 5 μm 19*150 mm; Mobile phase: A-water (0.1% FA): B-acetonitrile; Flow rate: 20 mL/min). The fraction was lyophilized to give the product LD-4-1 (TEA salt) (270 mg, 55% yield) as a light-yellow solid.

MS (ESI) m/z: 1077.4 [M+H]⁺

TABLE 33
Linker-Payload List

| Linker- | |
|---|---|
| Payload | Linker-Payload Structure |
| LD-1-A | 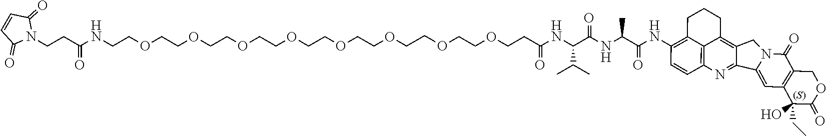 |
| LD-2-1 | 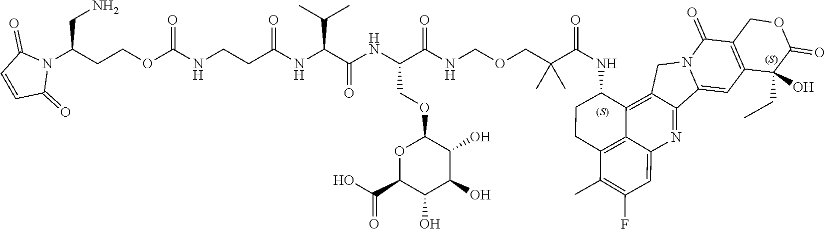 |
| LD-3-1 | 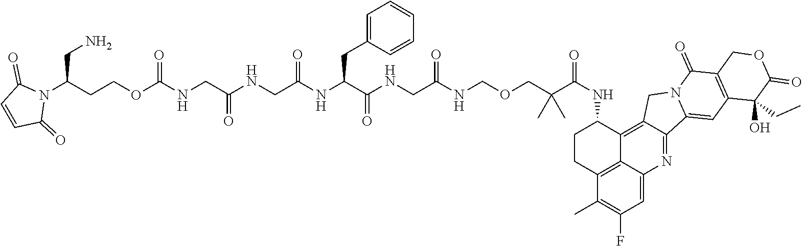 [SMILES: CC[C@@]1(O)C(=O)OCc2c1cc1n(c2=O)Cc2c-1nc1cc(F)c(C)c3c1c2[C@@H](NC(=O)C(C)(C)COCNC(=O)CNC(=O)[C@H](Cc1ccccc1)NC(=O)CNC(=O)CNC(=O)OCC[C@H](CN)N1C(=O)C=CC1=O)CC3] |
| LD-4-1 | 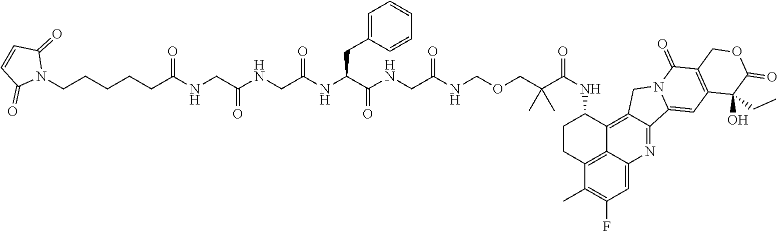 [SMILES: CC[C@@]1(O)C(=O)OCc2c1cc1n(c2=O)Cc2c-1nc1cc(F)c(C)c3c1c2[C@@H](NC(=O)C(C)(C)COCNC(=O)CNC(=O)[C@H](Cc1ccccc1)NC(=O)CNC(=O)CNC(=O)CCCCCN1C(=O)C=CC1=O)CC3] |

### Example 26: Antibody Drug Conjugate (ADC) Preparation and Characterization

Antibody information. In addition to the antibodies disclosed in Tables 30 and 31, QD6/B09-57 antibody was also prepared for conjugation. QD6/B09-57 (also referred to herein as BGX0) is the parental antibody of AZD9592 and it was generated following the procedure reported in Patent Application Publication No. US 2023/0183358 A1.

### QD6/B09-57 Sequence (Anti-EGFR×cMET Antibody)

Heavy chain 1 sequence
(SEQ ID NO: 182)
QVQLVQSGAEVKKPGSSVKVSCKASGGTFSDNDFSWVRQAPGQGLEWMGAI
VAVFRTETYAQKFQDRVKITADISTRTTYMELSSLRSEDTAVYYCARRLMSAISGPGA
PLLMWGQGTLVTVSSASTKGPSVCPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKS
VDKTHTCPPCPAPEFEGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPASIEK
TISKAKGQPREPQVYTLPPCREEMTKNQVSLWCLVKGFYPSDIAVEWESNGQPENNY
KTTPPVLDSDGSFFLYSKLTVDKSRWQQGNVFSCSVMHEALHNHYTQKSLSLSPGK
Heavy chain 2 sequence
(SEQ ID NO: 183)
QVQLVQSGAEVKKPGASVKVSCKASGYTFTDYYIHWVRQATGQGLEWMGW
MNPNSGNTGYAQKFQGRVTMTRDTSISTAYMELSSLRSEDTAVYYCARGQGYTHSW
GQGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKT
HTCPPCPAPEFEGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPASIEKTISK
AKGQPREPQVCTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSPGK
Light chain 1 sequence
(SEQ ID NO: 184)
QSALTQPRSVSGSPGQSVTISCTGTSSDVGGYNYVSWYQQHPGKAPKLMIYDV
SKRPSGVPDRFSGSKSGNTASLTISGLQAEDEADYYCSSYTSSDTLEIFGGGTKLTVLG
QPKAAPSVTLFPPCSEELQANKATLVCLISDFYPGAVTVAWKADSSPVKAGVETTTPS
KQSNNKYAASSYLSLTPEQWKSHRSYSCQVTHEGSTVEKTVAPTEVS
Light chain 2 sequence
(SEQ ID NO: 185)
DIQMTQSPSTLSASVGDRVTITCRASEGIYHWLAWYQQKPGKAPKLLIYKASSL
ASGVPSRFSGSGSGTEFTLTISSLQPDDFATYYCQQYSNYPPTFGGGTKLEIKRTVAAPS
VFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC

Reference ADC AZD9592 preparation. AZD9592 biosimilar ADC (ADC-A, Table 34) was prepared by following the conjugation procedure reported in Patent Application Publication No. US 2023/0183358 A1, with a drug-to-antibody ratio (DAR) of 6 and >90% SEC purity.

Antibody drug conjugate preparation. An antibody in a conjugation buffer (with concentration 0.5-25 mg/mL, PBS buffer pH 6.0-8.5) was incubated under reduction temperature (0-40° C.) for 10 min. 2-10 eq. TECP solution (5 mM stock in PBS buffer) was added to the reaction mixture and the reduction reaction was left for 1-24 hours at reduction temperature. Organic solvent (e.g., DMSO, DMF, DMA, PG, acetonitrile, 0-25% v/v) and the linker-payload stock prepared herein (5-25 eq, 10 mM stock in organic solvent) (see Table 33) were added stepwise after the reduction mixture was cooled to 0-25° C. Conjugation solution was left for 1-3 h at 0-25° C., and the reaction was quenched with N-acetyl cysteine (1 mM stock).

Maleimide hydrolysis after conjugation. After the conjugation step, the ADC was subjected to buffer exchange into ring opening buffer (pH 7.0-9.0, PBS, borate or tris buffer) and the solution was left at 22 or 37° C. for 5-48 h. The maleimide ring opening process was monitored via reduced LCMS. Once the conjugated maleimide hydrolysis was completed, the resulting ADCs were submitted to buffer exchange (spin desalting column, ultrafiltration, and dialysis) into storage buffer (e.g., pH 5.5-6.5 histidine acetate buffer, with optional additive such as sucrose, trehalose, tween 20, 60, 80).

ADC characterization. ADCs were characterized using the following analytical methods. Drug to antibody ratios (DAR) of the ADCs were determined by LCMS method or HIC method. SEC purity of ADCs made were all >95% purity and the free liker-payload residues in all ADC mixtures were all less than 2%.

LCMS method: LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of R=15 k and deconvolved using the ReSpect™ algorithm and Sliding Window integration in Thermo Scientific™ BioPharma Finder™ 4.0 software.

HIC method 1: HPLC analysis for DAR8 was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

HIC method 2: HPLC analysis for DAR6 was carried out under the following measurement conditions:
HPLC system: Waters ACQUITY ARC HPLC System
Detector: measurement wavelength: 280 nm
Column: Nanochrom BioCore HIC-Phenyl column, 5 μm, 4.6×100 mm
Column temperature: 30° C.
Mobile phase A: 1.2 M ammonium sulfate, 50 mM phosphate buffer, pH 6.0
Mobile phase B: 50 mM phosphate buffer, pH 6.0
Mobile phase C: isopropanol
Gradient program: 0% B-0% B and 2% C-2% C (0 min-5 min) 0% B-75% B and 2% C-15% C (5 min-40 min) 75% B-75% B and 15% C-15% C (40 min-44 min) 0% B-0% B and 2% C-2% C (44 min-50 min)
Injected sample amount: 20 μg

SEC method to determine ADC purity: HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM phosphate buffer, 250 mM potassium chloride, 15% isopropyl alcohol, pH 7.0
Gradient program: under 10 min isocratic elutions with the flow rate of 0.3 mL/min
Injected sample amount: 20 μg

TABLE 34
Antibody Drug Conjugates

| ADC No. | Antibody | ADC structure |
|---|---|---|
| ADC-A (Ref.) | QD6/ B09-57 (AZ bsAb) BGX0 | 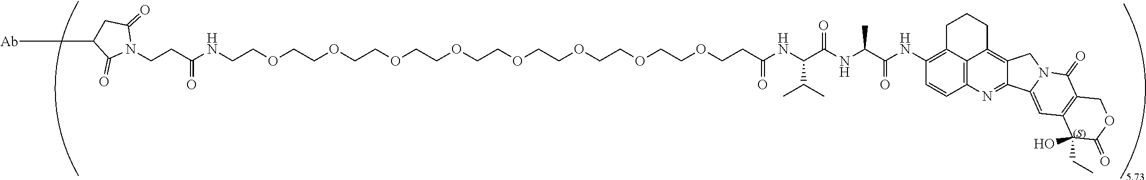 |
| ADC-1 | BG- T187 FE BGX2 | 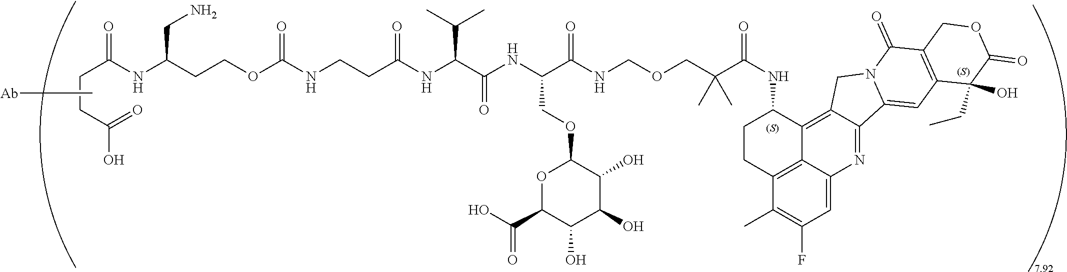 |
| ADC-2 | BG- T187 HE BGX3 | 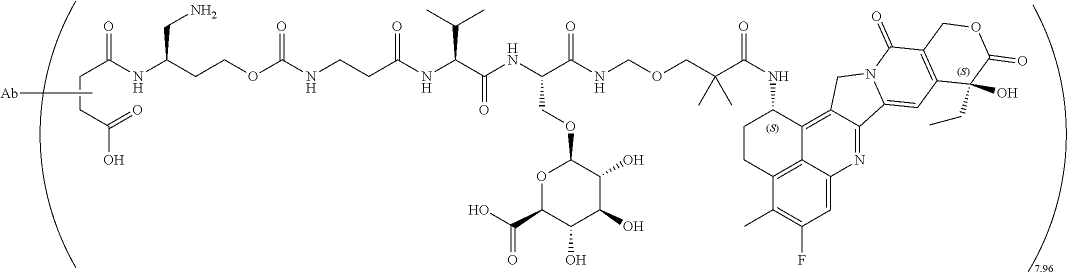 |
| ADC-3 | BG- T187 wt BGX1 | 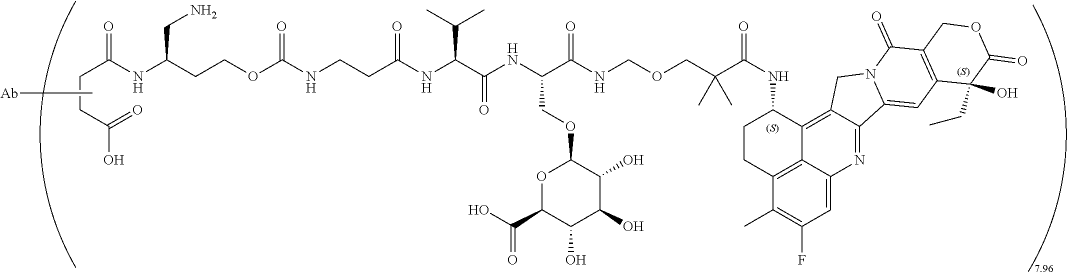 |
| ADC-4 | BG- T187 NE + YTE BGX5 | 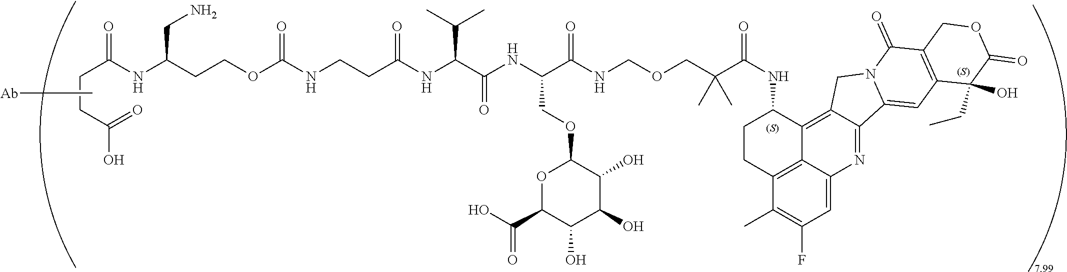 |
| ADC-5 | BG- T187 FE + YTE BGX4 | 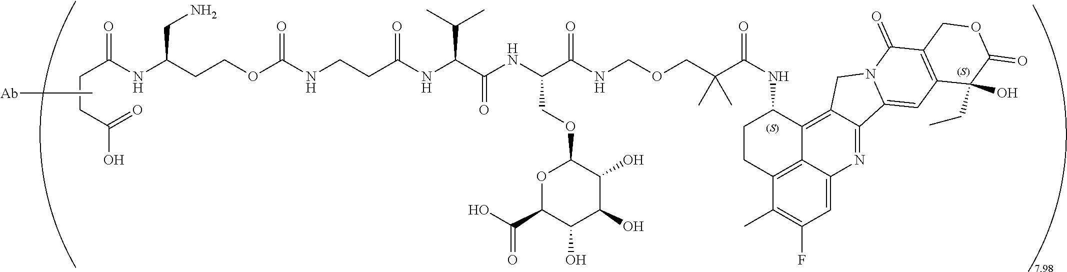 |
| ADC-6 | BG- T187 FE + YTE BGX4 | 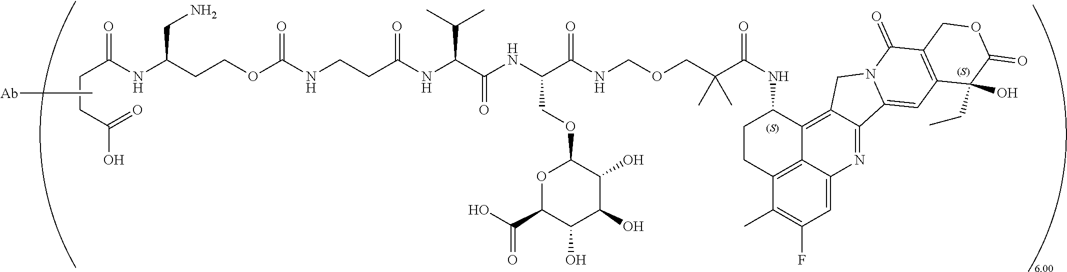 |

### Example 27: Cell Lines and Their cMET and EGFR Expression Levels

The following cell lines were obtained from ATCC and were used in the in vitro and in vivo experiments disclosed below. The complete growth medium for each cell line included fetal bovine serum (Gibco, 10099-141C) to a final concentration of 10% and 1% pen-strep (Gibco, 15140122). Each cell line was grown in a humidified 5% CO₂ atmosphere at 37° C.

NCI-H1975 (CRL-5908). H1975 is a cell line exhibiting epithelial morphology that was isolated from the lung tissue of a female patient with adenocarcinoma. The base medium for H1975 is RPMI-1640 (Gibco, 72400120).

HCC827 (CRL-2868). HCC827 is a cell line exhibiting epithelial morphology that was isolated from the lung tissue of a female patient with adenocarcinoma. The base medium for HCC827 is RPMI-1640.

NCI-H441 (CRM-HTB-174). H441 is an epithelial-like cell line that was isolated from the lung of a male with papillary adenocarcinoma. The base medium for H441 is RPMI-1640.

HT-29 (HTB-38). HT-29 is a cell line with epithelial morphology that was isolated from a primary tumor obtained from a 44-year-old, white, female patient with colorectal adenocarcinoma. The base medium for HT-29 is McCoy's 5A (Gibco, 36600021).

NCI-H520 (HTB-182). H520 was isolated from the lung tissue of a male squamous cell carcinoma patient. The base medium for H520 is RPMI-1640.

The cMET and EGFR expression levels of each cell line were determined by QSC kit and the levels are shown below in Table 35

TABLE 35
Cell Lines and Their cMET and EGFR Expression Levels

| Cell Line | cMET | EGFR |
|---|---|---|
| HCC827 | 157,257 | 111,516 |
| H1975 | 71,324 | 24,392 |
| H441 | 111,701 | 19,611 |
| HT29 | 37,627 | 21,626 |
| H520 | neg | neg |

### Example 28: ADC Internalization in EGFR- and cMET-Expressing Cell Lines HCC827 and H1975

ADC internalization into HCC827 and H1975 cells was studied using pHrodo dye (Invitrogen #p36011; 100 μg dissolved in 50 μL DMSO; ˜2 mM). ADCs to be labeled were prepared at a concentration of at least 1 mg/mL in 0.1 M sodium bicarbonate buffer (pH 8.3). An appropriate amount of the pHrodo reactive dye was added to the ADC solution and then mixed by pipetting up and down several times. The mixture was incubated for 15-60 minutes at room temperature while being protected from light. Then the ADCs were purified using Zeba Spin Desalting Columns (Invitrogen #87767).

HCC827 or H1975 cells were seeded at 90 μl/well into 96 well plates (Corning #3599) in complete cell culture medium with a target of -70% confluence at the time of ADC addition. ADCs were added at 10 μl/well with a final concentration of 20 μg/ml. The plates were transferred to an IncuCyte® Live Cell Analysis System at 37° C., 5% CO₂ and scanned every hour for 24 hours. Data was collected and analyzed by IncuCyte software.

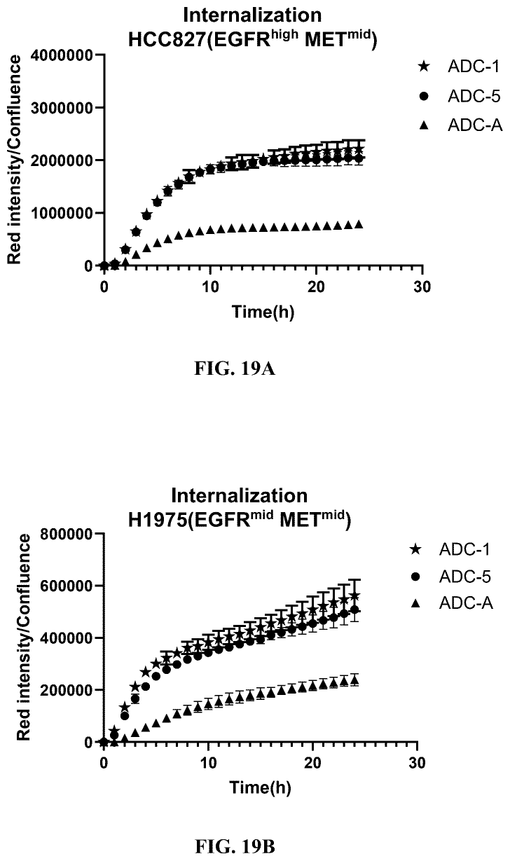

Results are shown in FIG. 19A (HCC827 cells) and FIG. 19B (H1975 cells). The results show that ADC-1 and ADC-5 were successfully internalized into cells, and were internalized with higher efficacy than was ADC-A.

### Example 29: Direct Killing in Cancer Cell Lines

Cells were seeded into 96-well plates (Corning #3903) at 90 μl/well and a concentration of 1E3 cells/well (HCC827 and HT-29) or 2E3 cells/well (H441 and H520). Plates were incubated at 37° C. and 5% C02 overnight. Then fresh complete culture medium containing varying concentrations of ADCs was added to the cells, and the cells were incubated at 37° C. and 5% CO₂ for 6 days. Cell viability was detected by adding 50 μl/well of CellTiter-Glo® reagent (Promega #G924B) and then shaking the plates at 400 rpm at room temperature for 30 minutes. Luminescence was read with a BMG PheraStar Reader.

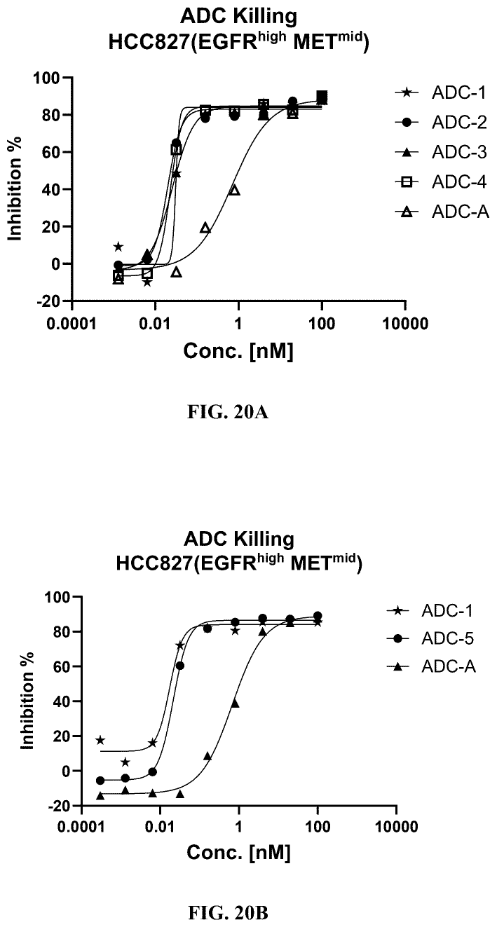

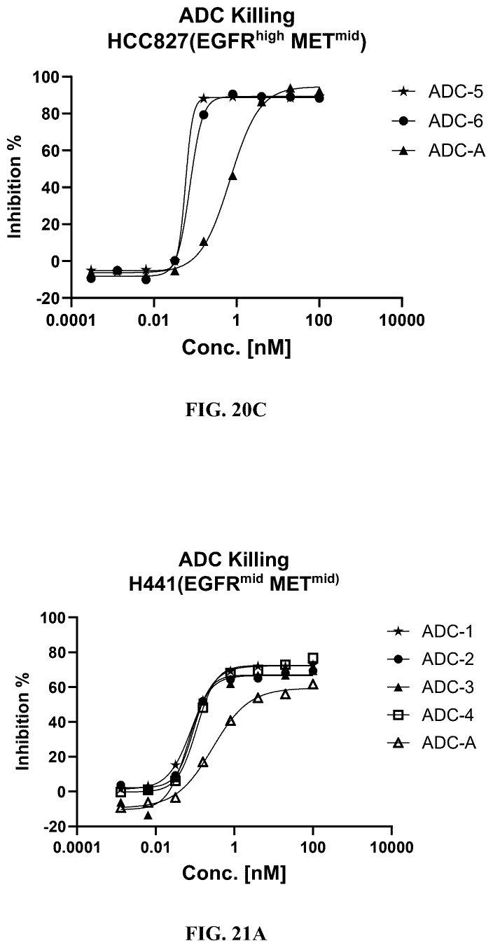

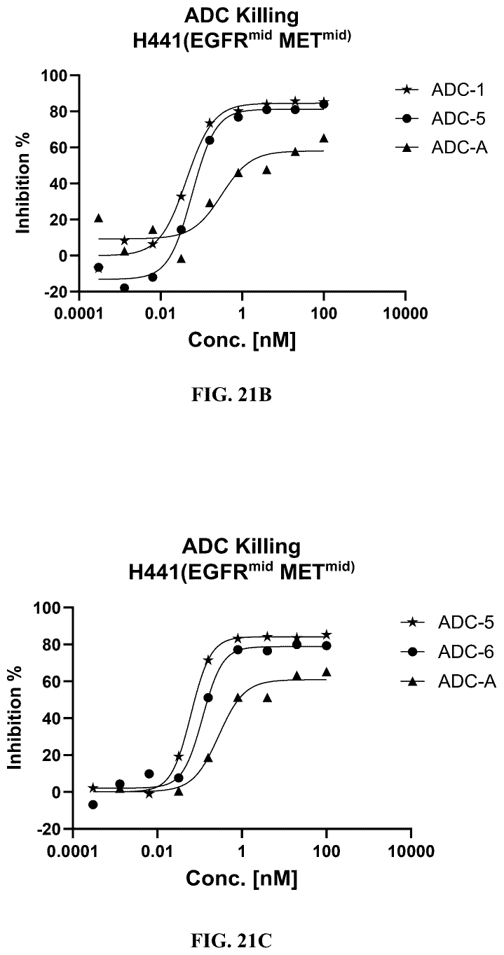

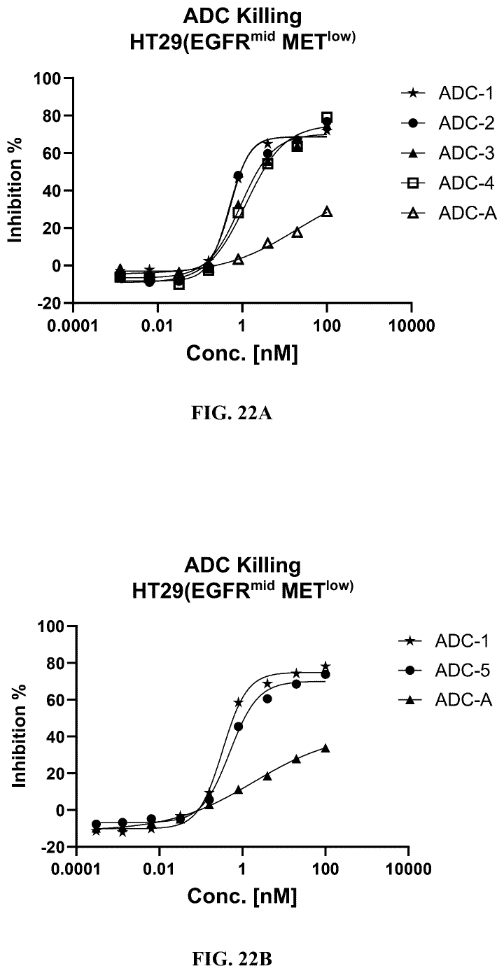

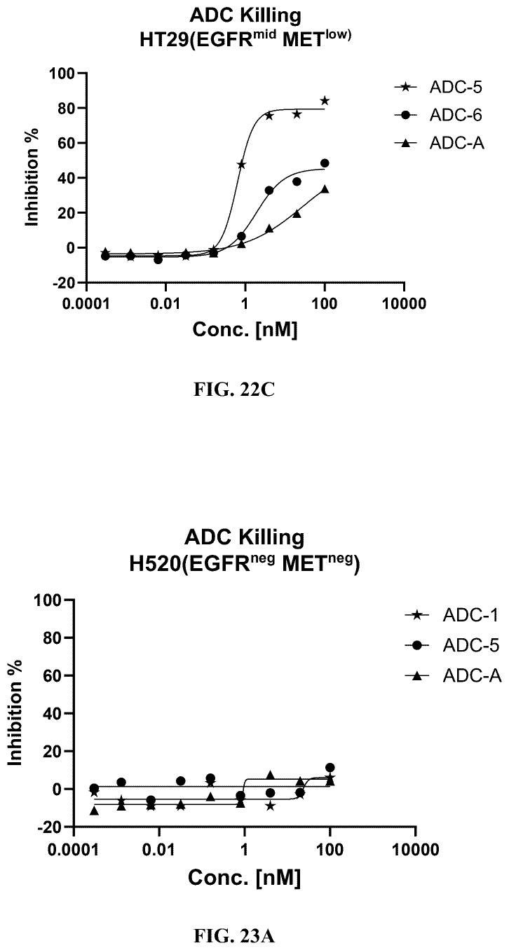

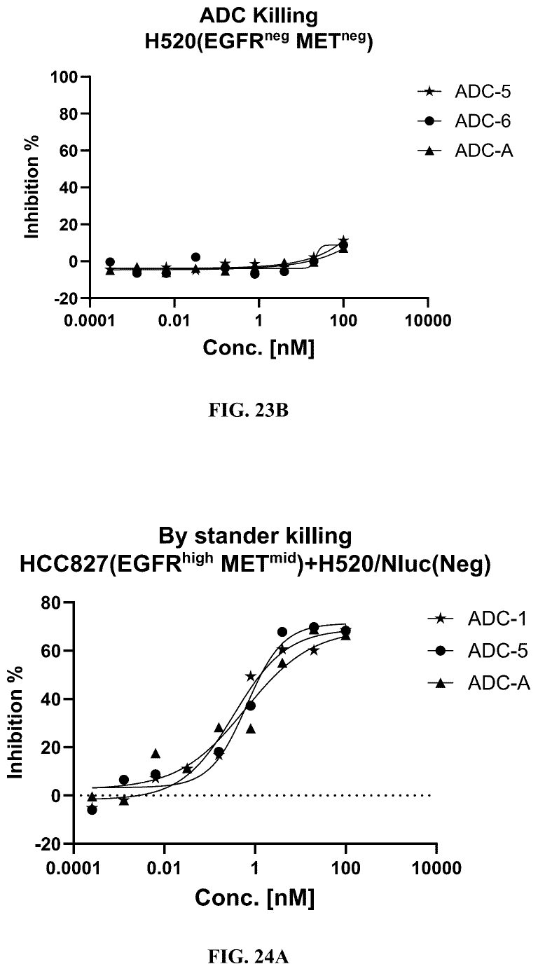

Results are shown in FIGS. 20A-23B and Tables 36 and 37A-B. The results show that the tested ADCs exhibited potent proliferation inhibition activity against HCC827 (FIGS. 20A-20C), H441 (FIGS. 21A-21C), and HT-29 (FIGS. 22A-22C) tumor cells, but not H520 (cMET^{neg} EGFR^{neg}) cells (FIGS. 23A-23B).

TABLE 36
ADC Anti-Proliferation Effects

| | HCC827 | | H441 | | HT29 | |
|---|---|---|---|---|---|---|
| | EGFR 111,516 | | EGFR 19,611 | | EGFR 21,626 | |
| | MET 157,257 | | MET 111,701 | | MET 37,627 | |
| ADC No. | IC50(nM) | Imax % | IC50(nM) | Imax % | IC50(nM) | Imax % |
| ADC-1 | 0.03 | 87.69 | 0.08 | 72.77 | 0.54 | 71.95 |
| ADC-2 | 0.02 | 90.33 | 0.09 | 69.39 | 0.48 | 76.82 |
| ADC-3 | 0.03 | 88.62 | 0.07 | 69.46 | 0.91 | 74.83 |
| ADC-4 | 0.02 | 90.12 | 0.11 | 76.79 | 1.23 | 79.03 |
| ADC-A | 0.71 | 88.30 | 0.28 | 61.61 | 18.65 | 28.81 |

TABLE 37A
ADC Anti-Proliferation Effects

| | HCC827 | | H441 | | HT29 | |
|---|---|---|---|---|---|---|
| | EGFR 111,516 | | EGFR 19,611 | | EGFR 21,626 | |
| | MET 157,257 | | MET 111,701 | | MET 37,627 | |
| ADCs | IC50(nM) | Imax % | IC50(nM) | Imax % | IC50(nM) | Imax % |
| ADC-1 | 0.02 | 84.51 | 0.04 | 85.56 | 0.33 | 78.17 |
| ADC-5 | 0.02 | 89.72 | 0.06 | 83.91 | 0.50 | 73.90 |
| ADC-A | 0.68 | 85.00 | 0.31 | 65.26 | 1.86 | 33.89 |

TABLE 37B
ADC Anti-Proliferation Effects

| | HCC827 | | H441 | | HT29 | |
|---|---|---|---|---|---|---|
| | EGFR 111,516 | | EGFR 19,611 | | EGFR 21,626 | |
| | MET 157,257 | | MET 111,701 | | MET 37,627 | |
| ADCs | IC50(nM) | Imax % | IC50(nM) | Imax % | IC50(nM) | Imax % |
| ADC-5 | 0.06 | 89.33 | 0.06 | 85.26 | 0.64 | 84.14 |
| ADC-6 | 0.07 | 90.58 | 0.12 | 79.90 | 1.90 | 48.47 |
| ADC-A | 0.69 | 93.82 | 0.28 | 65.23 | 7.76 | 33.75 |

### Example 30: ADC Bystander Killing in Co-Cultured HCC827 and H520-nanoLuc Cells

Method: H520-nanoLuc cell line construction. PT67-nanoLuc cells were cultured, then the cell-culture medium (containing the virus (nano-Luc gene)) was collected and filtered. H520 cells were seeded into 6-well plates at 1E5 cells/well, and incubated at 37° C., 5% CO₂, overnight. The PT67-nanoLuc cell medium and 8 μg/ml polybrene were added. The infection was repeated 3 times, every 24 hours. Then the H520-nanoLuc cells were cultured for selection with the addition of 1 mg/ml Geneticin for 5 days. The H520-nanoLuc cells were collected, and Nano-Glo reagent (Promega: N1120) was added to test the nano-Luc transfection efficiency.

Method: ADC bystander killing. HCC827 and H520-nanoLuc cells (2:1), or H520-nanoLuc cells alone were seeded into 96-well white plates (Corning #3903), and incubated at 37° C., 5% CO₂, overnight. Fresh cell culture medium containing the varying concentrations of ADCs was added at 10 μl/well. The cells were incubated at 37° C. and 5% CO₂ for 6 days. The supernatant was discarded.

The H520-nanoLuc cell viability was detected by Nano-Glo reagent (Promega #N1120). After addition, the plates were shaken at 400 rpm for 30 minutes at room temperature. Then the plates were read with a BMG PheraStar Reader.

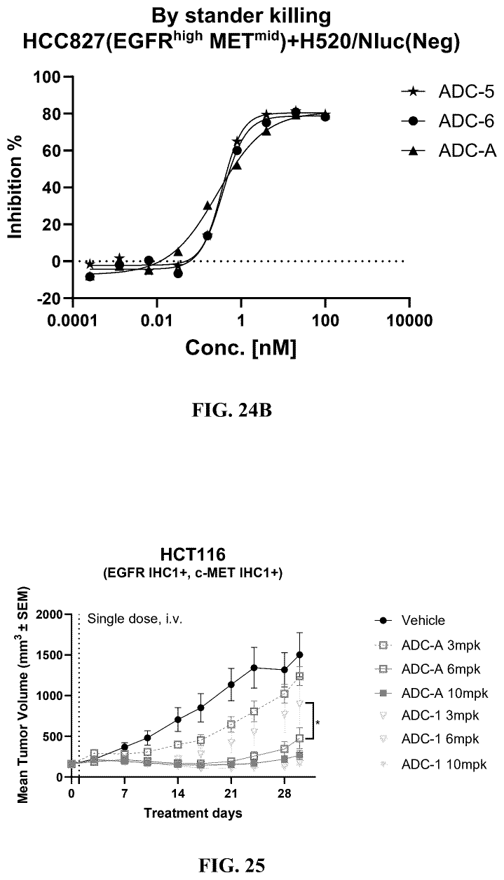

Results are shown in FIGS. 24A-24B and Table 38A-B.

TABLE 38A
ADC Bystander Killing

HCC827:H520-nanoLuc = 2:1

| ADC | Imax (%) | IC50 (nM) |
|---|---|---|
| ADC-1 | 0.33 | 68.53 |
| ADC-5 | 0.61 | 69.81 |
| ADC-A | 0.61 | 68.84 |

TABLE 38B
ADC Bystander Killing

HCC827:H520-nanoLuc = 2:1

| ADC | Imax (%) | IC50 (nM) |
|---|---|---|
| ADC-5 | 0.35 | 81.39 |
| ADC-6 | 0.37 | 80.85 |
| ADC-A | 0.28 | 79.37 |

### Example 31: In Vivo Efficacy of ADCs in Human Cancer Xenograft Models

The in vivo efficacies of ADC-1, ADC-5, and a reference ADC (ADC-A) were evaluated in mice transplanted with human colon cancer cells (HCT116 and HT-29) or human lung cancer cells (H1650, H1975, and H441).

Female BALB/c nude mice were subcutaneously implanted with 3×10⁶ to 5×10⁶ cells per 200 μL PBS with 50% Matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 150-250 mm³, mice with tumors were randomly allocated into 5-7 groups with 8 animals in each group, and were intravenously treated with single dose of vehicle, ADC-1, ADC-5, or ADC-A at 1, 3, 6, or 10 mg/kg. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

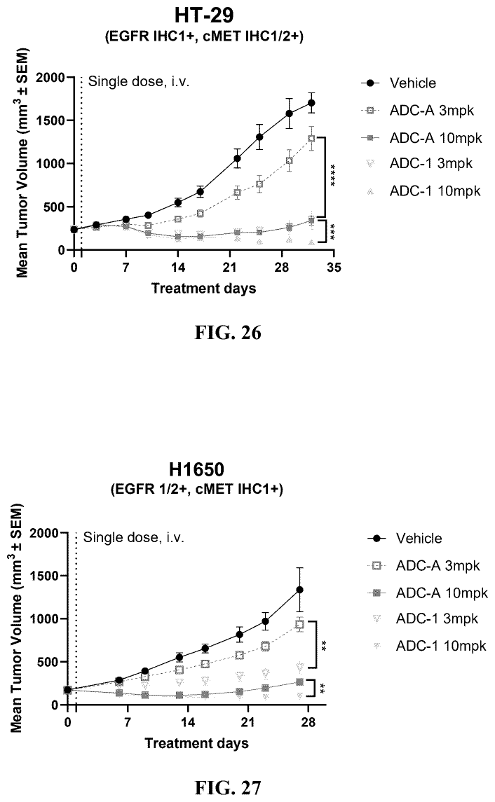

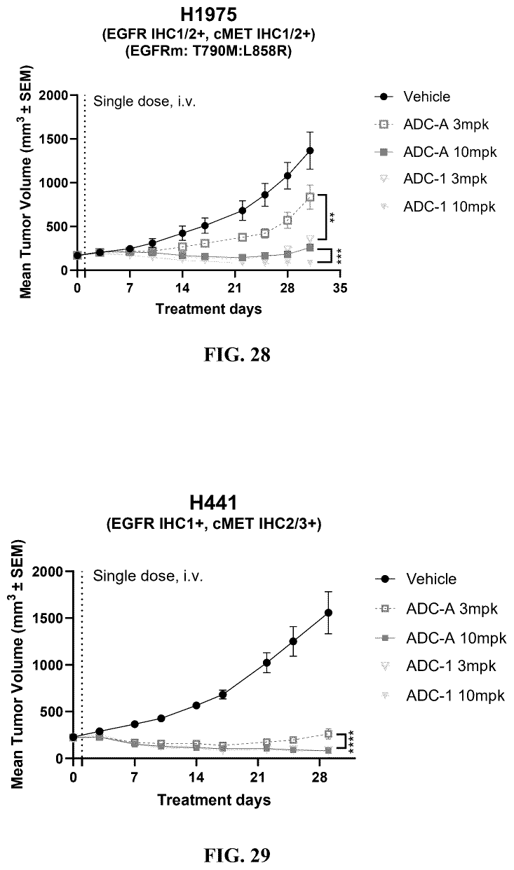

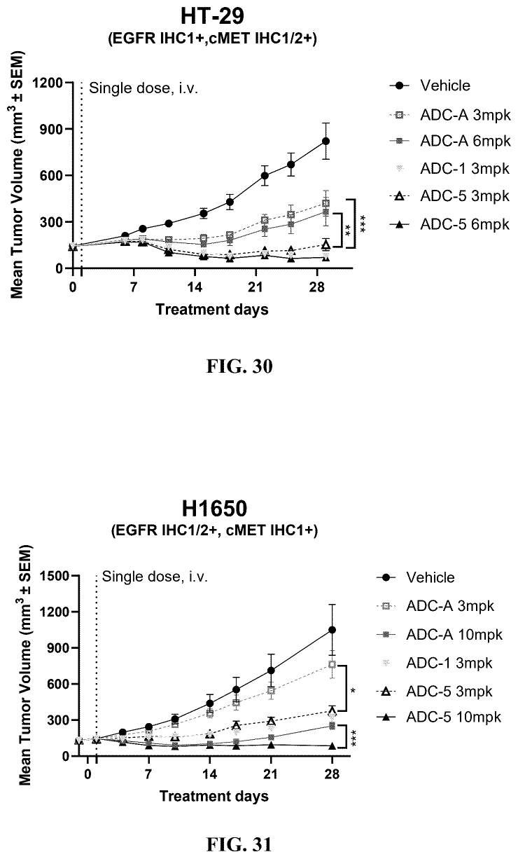

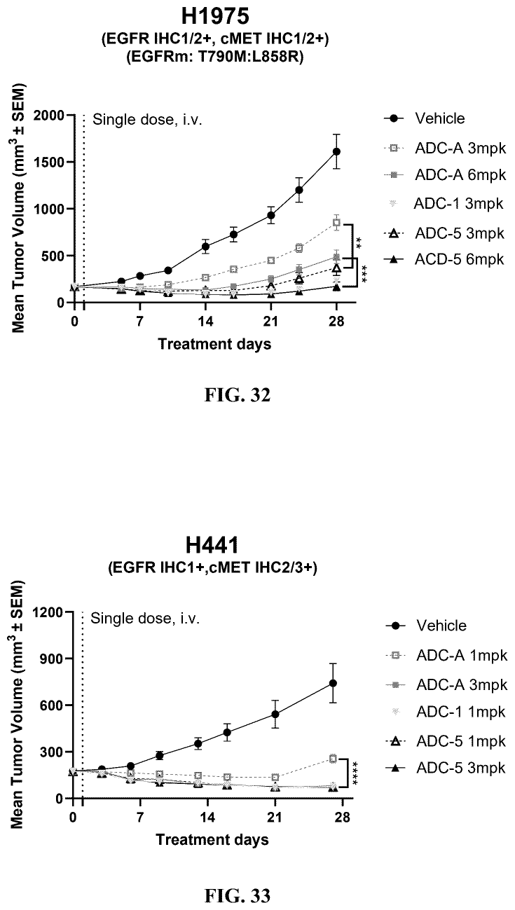

Data is presented as mean tumor volume±standard error of the mean (SEM). Results are presented in FIGS. 25-33 and Tables 39-47. The results demonstrate that each of ADC-1 and ADC-5 showed robust anti-tumor activity in human colon (ADC-1, FIGS. 25-26, Tables 39-40 and 44; ADC-5, FIG. 30, Table 44) and lung (ADC-1, FIGS. 27-29, Tables 41-43 and 45-47; ADC-5, FIGS. 31-33, Tables 45-47) tumor xenograft models with EGFR and cMET double low/medium expression. ADC-1 and ADC-5 demonstrated comparable efficacy (FIGS. 30-33). Each of ADC-1 and ADC-5 exhibited better anti-tumor activity than did reference ADC-A at the same dose (FIGS. 25-33). ADC-5 exhibited better anti-tumor activity than did reference ADC-A at a lower dose in HT-29 human colon cancer xenograft model (FIG. 30).

TABLE 39
ADC Efficacy in HCT116 Colon Cancer Xenograft Model

| Treatment | TGI on Day 30 | PR |
|---|---|---|
| ADC-A 3 mpk | 20 | N/A |
| ADC-A 6 mpk | 77 | N/A |
| ADC-A 10 mpk | 92 | N/A |
| ADC-1 3 mpk | 45 | N/A |
| ADC-1 6 mpk | 99 | 1/7 |
| ADC-1 10 mpk | 92 | 2/7 |

TABLE 40
ADC Efficacy in HT-29 Colon Cancer Xenograft Model

| Treatment | TGI on Day 32 | PR |
|---|---|---|
| ADC-A 3 mpk | 28 | N/A |
| ADC-A 10 mpk | 93 | N/A |
| ADC-1 3 mpk | 93 | 1/8 |
| ADC-1 10 mpk | 110 | 2/8 |

TABLE 41
ADC Efficacy in H1650 Lung Cancer Xenograft Model

| Treatment | TGI on Day 27 | PR |
|---|---|---|
| ADC-A 3 mpk | 34 | N/A |
| ADC-A 10 mpk | 92 | N/A |
| ADC-1 3 mpk | 78 | N/A |
| ADC-1 10 mpk | 106 | 3/8 |

TABLE 42
ADC Efficacy in H1975 Lung Cancer Xenograft Model

| Treatment | TGI on Day 31 | PR |
|---|---|---|
| ADC-A 3 mpk | 46 | N/A |
| ADC-A 10 mpk | 92 | N/A |
| ADC-1 3 mpk | 85 | N/A |
| ADC-1 10 mpk | 107 | 4/8 |

TABLE 43
ADC Efficacy in H441 Lung Cancer Xenograft Model

| Treatment | TGI on Day 29 | PR |
|---|---|---|
| ADC-A 3 mpk | 97 | N/A |
| ADC-A 10 mpk | 111 | 6/8 |
| ADC-1 3 mpk | 110 | 5/8 |
| ADC-1 10 mpk | 110 | 5/8 |

TABLE 44
ADC Efficacy in HT-29 Colon Cancer Xenograft Model

| Treatment | TGI on Day 29 | PR or CR |
|---|---|---|
| ADC-A 3 mpk | 59 | N/A |
| ADC-A 6 mpk | 67 | N/A |
| ADC-1 3 mpk | 110 | 1/8 |
| ADC-5 3 mpk | 98 | 2/8 |
| ADC-5 6 mpk | 111 | 2/8 PR, 1/8 CR |

TABLE 45
ADC Efficacy in H1650 Lung Cancer Xenograft Model

| Treatment | TGI on Day 28 |
|---|---|
| ADC-A 3 mpk | 32 |
| ADC-A 10 mpk | 88 |
| ADC-1 3 mpk | 81 |
| ADC-5 3 mpk | 74 |
| ADC-5 10 mpk | 107 |

TABLE 46
ADC Efficacy in H1975 Lung Cancer Xenograft Model

| Treatment | TGI on Day 28 | PR |
|---|---|---|
| ADC-A 3 mpk | 53 | N/A |
| ADC-A 6 mpk | 78 | N/A |
| ADC-1 3 mpk | 96 | N/A |
| ADC-5 3 mpk | 86 | 1/8 |
| ADC-5 6 mpk | 100 | 3/8 |

TABLE 47
ADC Efficacy in H441 Lung Cancer Xenograft Model

| Treatment | TGI on Day 27 | PR |
|---|---|---|
| ADC-A 1 mpk | 85 | N/A |
| ADC-A 3 mpk | 117 | 3/8 |
| ADC-1 1 mpk | 118 | 4/8 |
| ADC-5 1 mpk | 118 | 3/8 |
| ADC-5 3 mpk | 118 | 4/8 |

1-81. (canceled)

82. An antibody drug conjugate, comprising: a multispecific antibody or antigen-binding fragment thereof, comprising
a first antigen binding domain that specifically binds to a first epitope of human cMET;
a second antigen binding domain that specifically binds to a second epitope of human cMET; and
a third antigen binding domain that specifically binds to human EGFR;
wherein the first epitope is distinct from the second epitope, or wherein the first antigen binding domain does not compete with the second antigen binding domain for binding to cMET;
wherein the antibody drug conjugate has the Formula A-I: 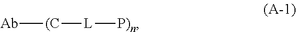
or a pharmaceutically acceptable salt, solvate, or hydrate thereof wherein:
Ab is the multispecific antibody or antigen-binding fragment thereof;
C is a conjugator;
L is a linker;
P is a payload; and
n is from 1 to 15.

83. The antibody drug conjugate of claim 82, wherein the first antigen binding domain that specifically binds to the first epitope of human cMET comprises:
(1) a heavy chain variable region that comprises: (a) a HCDR1 (Heavy Chain Complementarity Determining Region 1) of SEQ ID NO: 6, (b) a HCDR2 of SEQ ID NO: 7, (c) a HCDR3 of SEQ ID NO: 92 and a light chain variable region that comprises: (d) a LCDR1 (Light Chain Complementarity Determining Region 1) of SEQ ID NO: 66, (e) a LCDR2 of SEQ ID NO: 67, and (f) a LCDR3 of SEQ ID NO: 68;
(2) a heavy chain variable region that comprises: (a) a HCDR1 of SEQ ID NO: 6, (b) a HCDR2 of SEQ ID NO: 7, (c) a HCDR3 of SEQ ID NO: 90 and a light chain variable region that comprises: (d) a LCDR1 of SEQ ID NO: 66, (e) a LCDR2 of SEQ ID NO: 67, and (f) a LCDR3 of SEQ ID NO: 68; or
(3) a heavy chain variable region that comprises: (a) a HCDR1 of SEQ ID NO: 6, (b) a HCDR2 of SEQ ID NO: 7, (c) a HCDR3 of SEQ ID NO: 88 and a light chain variable region that comprises: (d) a LCDR1 of SEQ ID NO: 66, (e) a LCDR2 of SEQ ID NO: 67, and (f) a LCDR3 of SEQ ID NO: 68;

84. The antibody drug conjugate of claim 82, wherein the first antigen binding domain that specifically binds to the first epitope of human cMET comprises:
(1) a heavy chain variable region (VH) comprising an amino acid sequence at least 95% identical to SEQ ID NO: SEQ ID NO: 173, and a light chain variable region (VL) comprising an amino acid sequence at least 95% identical to SEQ ID NO: 64; or
(2) a heavy chain variable region (VH) comprising an amino acid sequence at least 95% identical to SEQ ID NO: 174, and a light chain variable region (VL) comprising an amino acid sequence at least 95% identical to SEQ ID NO: 64.

85. The antibody drug conjugate of claim 82, wherein the second antigen binding domain that specifically binds to the second epitope of human cMET comprises:
a heavy chain variable region that comprises: (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68.

86. The antibody drug conjugate of claim 82, wherein the second antigen binding domain that specifically binds to the second epitope of human cMET comprises:
a heavy chain variable region (VH) comprising an amino acid sequence at least 95% identical to SEQ ID NO: 145, and a light chain variable region (VL) comprising an amino acid sequence at least 95% identical to SEQ ID NO: 64.

87. The antibody drug conjugate of claim 82, wherein the third antigen binding domain that specifically binds to human EGFR comprises:
a heavy chain variable region that comprises: (a) a HCDR of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

88. The antibody drug conjugate of claim 82, wherein the third antigen binding domain that specifically binds to human EGFR comprises a heavy chain variable region (VH) comprising an amino acid sequence at least 95% identical to SEQ ID NO: 142, and/or a light chain variable region (VL) comprising an amino acid sequence at least 95% identical to SEQ ID NO: 143.

89. The antibody drug conjugate of claim 82, wherein
the first antigen binding domain that specifically binds to the first epitope of human cMET comprises: a heavy chain variable region that comprises: (a) a HCDR1 of SEQ ID NO: 6, (b) a HCDR2 of SEQ ID NO: 7, (c) a HCDR3 of SEQ ID NO: 90 and a light chain variable region that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises: a heavy chain variable region that comprises: (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and f a LCDR3 of SEQ ID NO: 68; and
the third antigen binding domain that specifically binds to human EGFR comprises: a heavy chain variable region that comprises: (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

90. The antibody drug conjugate of claim 82, wherein
the first antigen binding domain that specifically binds to the first epitope of human cMET comprises: a heavy chain variable region that comprises: (a) a HCDR1 of SEQ ID NO: 6, (b) a HCDR2 of SEQ ID NO: 7, (c) a HCDR3 of SEQ ID NO: 92 and a light chain variable region that comprises: (d) a LCDR1 of SEQ ID NO: 66, (e) a LCDR2 of SEQ ID NO: 67, and (f) a LCDR3 of SEQ ID NO: 68;
the second antigen binding domain that specifically binds to the second epitope of human cMET comprises: a heavy chain variable region that comprises: (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises: a heavy chain variable region that comprises: (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

91. The antibody drug conjugate of claim 82, wherein
the first antigen binding domain that specifically binds to the first epitope of human cMET comprises: a heavy chain variable region that comprises: (a) a HCDR1 of SEQ ID NO: 6, (b) a HCDR2 of SEQ ID NO: 7, (c) a HCDR3 of SEQ ID NO: 88 and a light chain variable region that comprises: (d) a LCDR1 of SEQ ID NO: 66, (e) a LCDR2 of SEQ ID NO: 67, and f a LCDR3 of SEQ ID NO: 68;
the second antigen binding domain that specifically binds to the second epitope of human cMET comprises: a heavy chain variable region that comprises: (a) a HCDR1 of SEQ ID NO: 56, (b) a HCDR2 of SEQ ID NO: 57, (c) a HCDR3 of SEQ ID NO: 58 and a light chain variable region that comprises: (d) a LCDR1 of SEQ ID NO: 66, (e) a LCDR2 of SEQ ID NO: 67, and (f) a LCDR3 of SEQ ID NO: 68; and
the third antigen binding domain that specifically binds to human EGFR comprises: a heavy chain variable region that comprises: (a) a HCDR1 of SEQ ID NO: 155, (b) a HCDR2 of SEQ ID NO: 156, (c) a HCDR3 of SEQ ID NO: 157 and a light chain variable region that comprises: (d) a LCDR1 of SEQ ID NO: 158, (e) a LCDR2 of SEQ ID NO: 159, and (f) a LCDR3 of SEQ ID NO: 160.

92. The antibody drug conjugate of claim 82, wherein
the first antigen binding domain that specifically binds to the first epitope of human cMET comprises: a heavy chain variable region (VH) comprising the amino acid sequence of SEQ ID NO: 173, and a light chain variable region (VL) comprising the amino acid sequence of SEQ ID NO: 64;
the second antigen binding domain that specifically binds to the second epitope of human cMET comprises: a heavy chain variable region (VH) comprising the amino acid sequence of SEQ ID NO: 145, and a light chain variable region (VL) comprising the amino acid sequence of SEQ ID NO: 64; and
the third antigen binding domain that specifically binds to human EGFR comprises: a heavy chain variable region (VH) comprising the amino acid sequence of SEQ ID NO: 142, and a light chain variable region (VL) comprising the amino acid sequence of SEQ ID NO: 143.

93. The antibody drug conjugate of claim 82, wherein
the first antigen binding domain that specifically binds to the first epitope of human cMET comprises: a heavy chain variable region (VH) comprising the amino acid sequence of SEQ ID NO: 174, and a light chain variable region (VL) comprising the amino acid sequence of SEQ ID NO: 64;
the second antigen binding domain that specifically binds to the second epitope of human cMET comprises: a heavy chain variable region (VH) comprising the amino acid sequence of SEQ ID NO: 145, and a light chain variable region (VL) comprising the amino acid sequence of SEQ ID NO: 64; and
the third antigen binding domain that specifically binds to human EGFR comprises: a heavy chain variable region (VH) comprising the amino acid sequence of SEQ ID NO: 142, and a light chain variable region (VL) comprising the amino acid sequence of SEQ ID NO: 143.

94. The antibody drug conjugate of claim 82, wherein
the first antigen binding domain that specifically binds to the first epitope of human cMET and the second antigen binding domain that specifically binds to the second epitope of human cMET comprise a bispecific IgG antibody, and wherein the third antigen binding domain that specifically binds to human EGFR comprises a single chain Fv (scFv),
wherein
(a) the scFv is attached to the C-terminal of a light chain of one or both of the first and second antigen binding domains;
(b) the scFv is attached to the N-terminal of a light chain of one or both of the first and second antigen binding domains;
(c) the scFv is attached to the N-terminal of a heavy chain of both of the first and second antigen binding domains;
(d) the scFv is attached to the N-terminal of a heavy chain of one of the first and second antigen binding domains;
(e) the scFv is attached to the C-terminal of a heavy chain constant region of one of the first and second antigen binding domains; or
(f) the scFv is attached to the C-terminal of a heavy chain constant region of both of the first and second antigen binding domains.

95. The antibody drug conjugate of claim 82, wherein
the third antigen binding domain that specifically binds to human EGFR comprises a scFv comprising a VH comprising the amino acid sequence of SEQ ID NO: 142 and a VL comprising the amino acid sequence of SEQ ID NO: 143;
wherein the VH and VL are connected via a first amino acid linker;
wherein the first amino acid linker comprises the amino acid sequence of any one of SEQ ID NOs: 97-139.

96. The antibody drug conjugate of claim 95, wherein the scFv comprises a first amino acid linker comprising the sequence of SEQ ID NO: 139 and wherein the third antigen binding domain that specifically binds to human EGFR comprises a scFv comprising the amino acid sequence of SEQ ID NO: 161.

97. The antibody drug conjugate of claim 82, wherein the first antigen binding domain that specifically binds to the first epitope of human cMET and the second antigen binding domain that specifically binds to the second epitope of human cMET collectively comprise a bispecific IgG antibody that comprises a human IgG1, IgG2, IgG3, or IgG4 heavy chain constant region, and/or a human kappa or lambda light chain constant region, wherein the heavy chain constant region optionally lacks a C-terminal lysine or a C-terminal glycine-lysine.

98. The antibody drug conjugate of claim 82, wherein the multispecific antibody or antigen-binding fragment thereof has antibody dependent cellular cytotoxicity (ADCC), antibody-dependent cellular phagocytosis (ADCP) or complement dependent cytotoxicity (CDC).

99. The antibody drug conjugate of claim 82, comprising a first polypeptide, a second polypeptide, and a third polypeptide, wherein
(1) the first polypeptide comprises the amino acid sequence of SEQ ID NO: 153, the second polypeptide comprises the amino acid sequence of SEQ ID NO: 150, and the third polypeptide comprises the amino acid sequence of SEQ ID NO: 148;
(2) the first polypeptide comprises the amino acid sequence of SEQ ID NO: 168, the second polypeptide comprises the amino acid sequence of SEQ ID NO: 169, and the third polypeptide comprises the amino acid sequence of SEQ ID NO: 148;
(3) the first polypeptide comprises the amino acid sequence of SEQ ID NO: 153, the second polypeptide comprises the amino acid sequence of SEQ ID NO: 170, and the third polypeptide comprises the amino acid sequence of SEQ ID NO: 148;
(4) the first polypeptide comprises the amino acid sequence of SEQ ID NO: 168, the second polypeptide comprises the amino acid sequence of SEQ ID NO: 171, and the third polypeptide comprises the amino acid sequence of SEQ ID NO: 148; or
(5) the first polypeptide comprises the amino acid sequence of SEQ ID NO: 153, the second polypeptide comprises the amino acid sequence of SEQ ID NO: 172, and the third polypeptide comprises the amino acid sequence of SEQ ID NO: 148.

100. The antibody drug conjugate of claim 82, wherein the antibody drug conjugate has Formula (I): 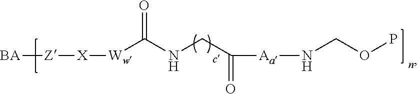
or a pharmaceutically acceptable salt, tautomer, solvate, or stereoisomer thereof, wherein:
BA is Ab;
Z′ is 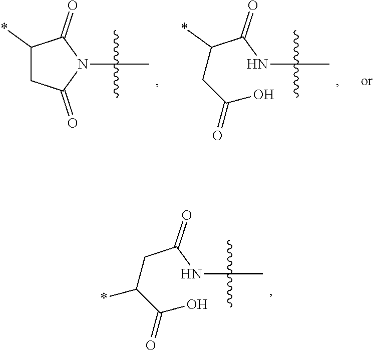 [SMILES: *C1CC(=O)N(*)C1=O ; *NC(=O)C(*)CC(=O)O ; *NC(=O)CC(*)C(=O)O]
wherein * indicates the bond where Z′ attaches to BA;
X is a substituted or unsubstituted C₅ alkyl;
W is oxygen;
w′ is 0 or 1;
c′ is 1 or 2;
a′ is 1, 2, or 3;
each A is, independently, an amino acid residue optionally substituted with a hydrophilic group;
P is a payload residue; and
n is from 1 to 15.

101. The antibody drug conjugate of claim 100, wherein X is —(CH₂)₃—, —(CH₂)₅—, —CH(CH₂NH₂)(CH₂)₂—, —CH(CH₂NH₂)(CH₂)₄—, —CH₂CH₁(NH₂)CH₂—, CH(CH₂NH₂)CH₂—, or —CH(CH₂NH₂)(CH₂)₂—.

102. The antibody drug conjugate of claim 101, wherein A is substituted with a hydrophilic group (HG) and HG is:

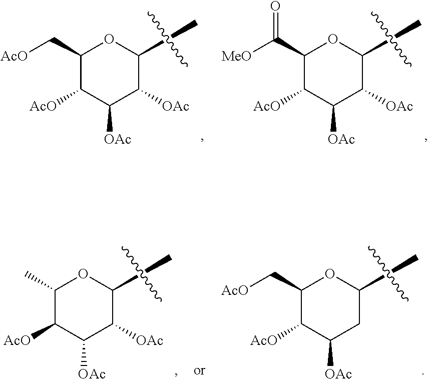

103. The antibody drug conjugate of claim 101, wherein A is 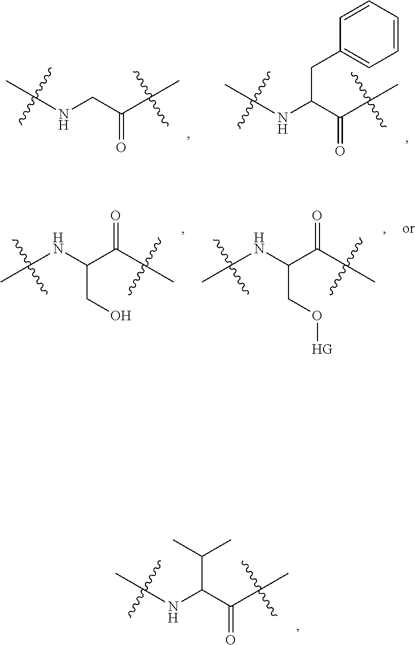
wherein HG is a hydrophilic group.

104. The antibody drug conjugate of claim 101, wherein A is 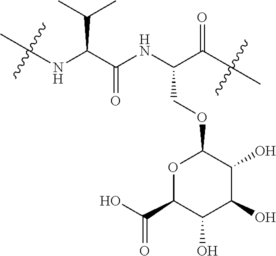 [SMILES: *N[C@H](C(=O)N[C@@H](CO[C@@H]1O[C@H](C(=O)O)[C@@H](O)[C@H](O)[C@H]1O)C(*)=O)C(C)C]
or wherein A is

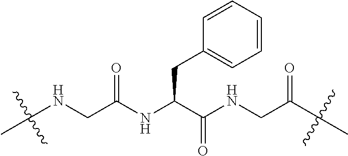 [SMILES: *NCC(=O)N[C@@H](Cc1ccccc1)C(=O)NCC(*)=O]

105. The antibody drug conjugate of claim 82, wherein P is:

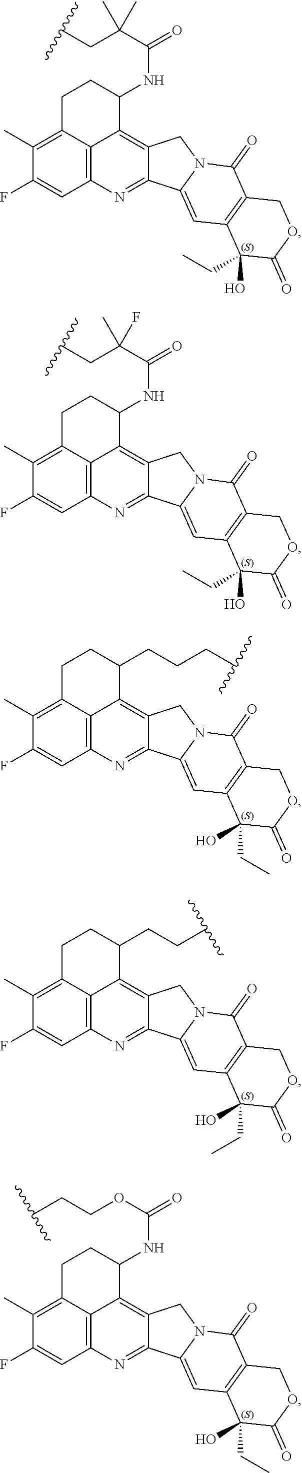

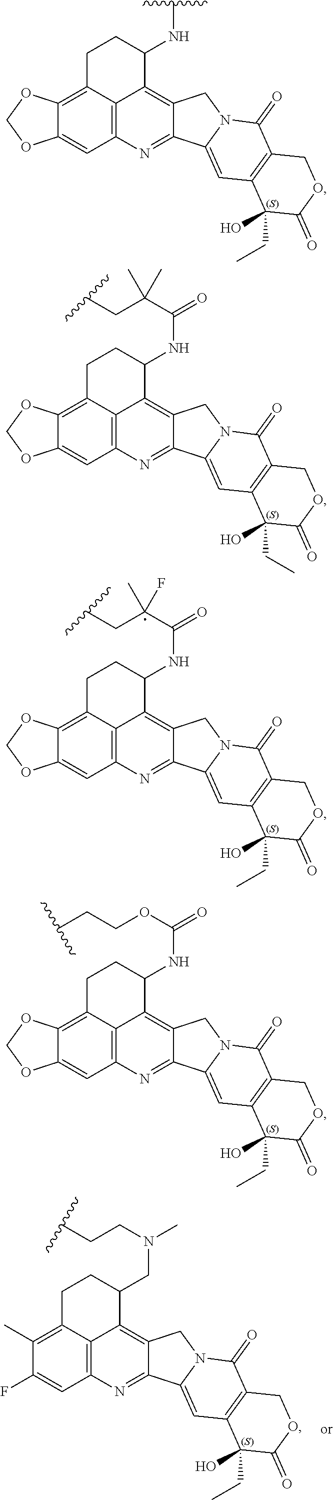

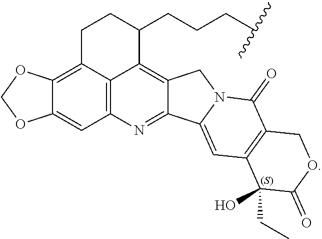

106. The antibody drug conjugate of claim 82, wherein the antibody drug conjugate is represented by one of the following formulas:
or a pharmaceutically acceptable salt, solvate, and/or stereoisomer thereof, wherein n is from 3 to 12.

107. The antibody drug conjugate of claim 82, wherein the antibody drug conjugate comprises one of the following formulas: 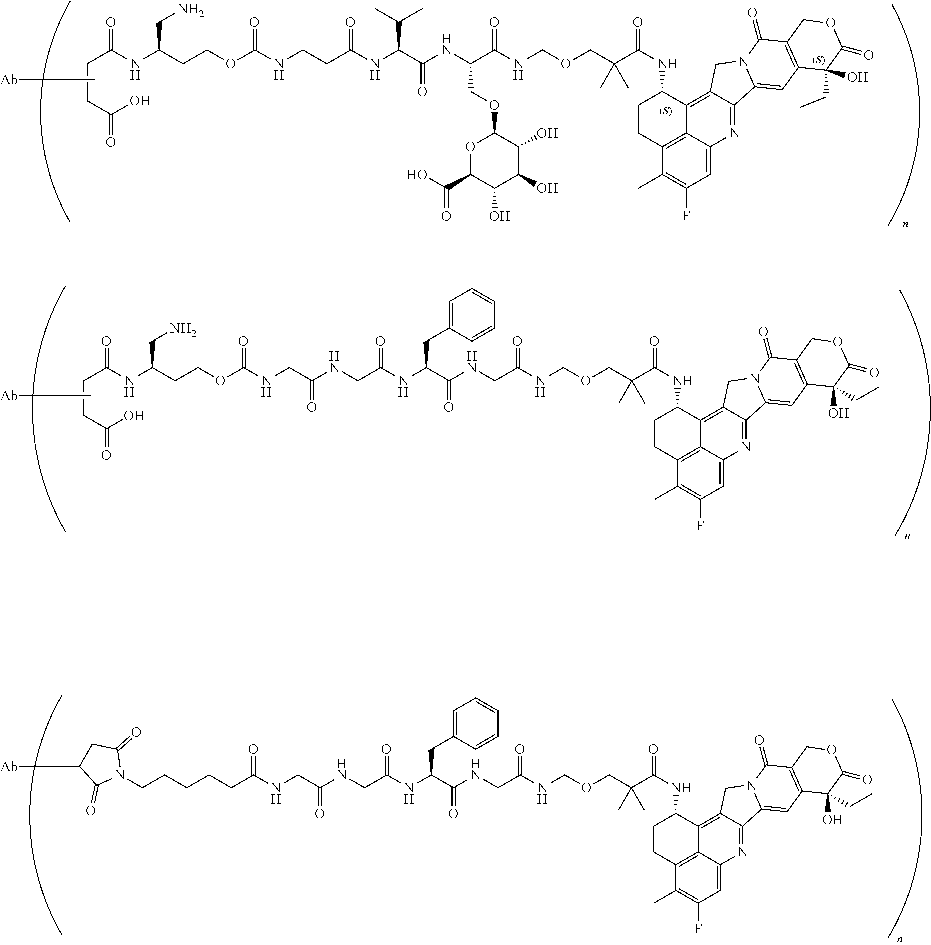 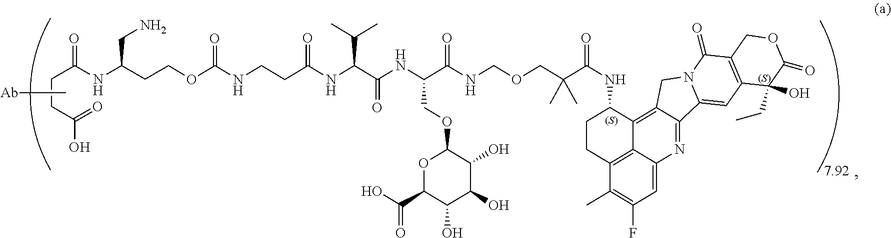
or a pharmaceutically acceptable salt, solvate, and/or stereoisomer thereof, wherein the Ab comprises a first polypeptide comprising the amino acid sequence of SEQ ID NO: 153, a second polypeptide comprising the amino acid sequence of SEQ ID NO: 170, and a third polypeptide comprising the amino acid sequence of SEQ ID NO: 148; 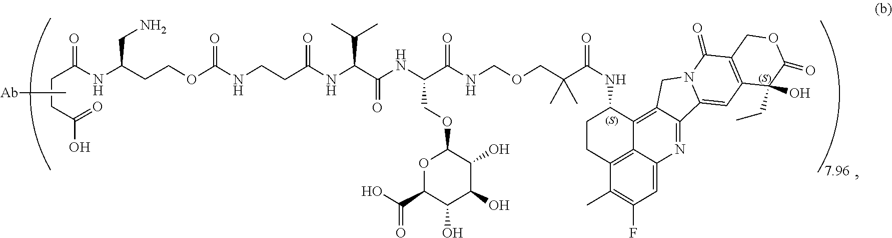
or a pharmaceutically acceptable salt, solvate, and/or stereoisomer thereof, wherein the Ab comprises a first polypeptide comprising the amino acid sequence of SEQ ID NO: 153, a second polypeptide comprising the amino acid sequence of SEQ ID NO: 172, and a third polypeptide comprising the amino acid sequence of SEQ ID NO: 148; 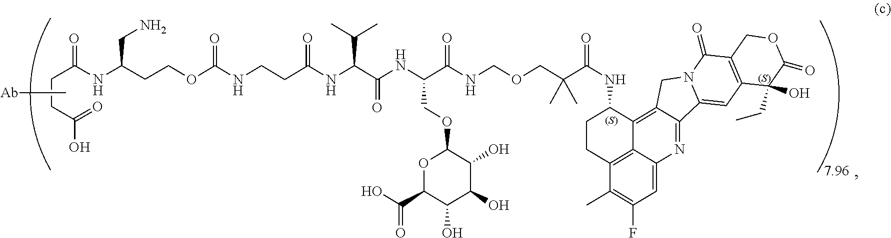
or a pharmaceutically acceptable salt, solvate, and/or stereoisomer thereof, wherein the Ab comprises a first polypeptide comprising the amino acid sequence of SEQ ID NO: 153, a second polypeptide comprising the amino acid sequence of SEQ ID NO: 150, and a third polypeptide comprising the amino acid sequence of SEQ ID NO: 148; 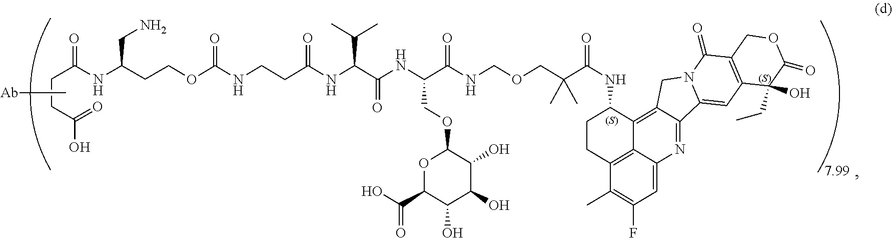
or a pharmaceutically acceptable salt, solvate, and/or stereoisomer thereof, wherein the Ab comprises a first polypeptide comprising the amino acid sequence of SEQ ID NO: 168, a second polypeptide comprising the amino acid sequence of SEQ ID NO: 169, and a third polypeptide comprising the amino acid sequence of SEQ ID NO: 148; 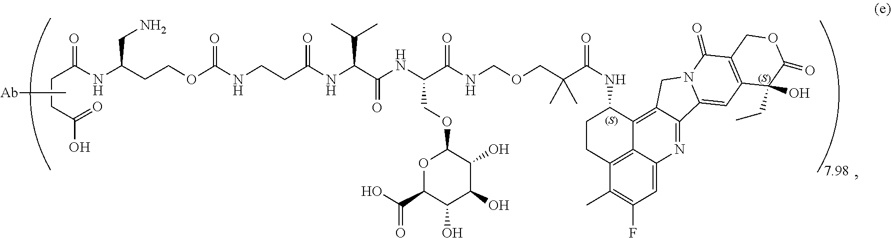
or a pharmaceutically acceptable salt, solvate, and/or stereoisomer thereof, wherein the Ab comprises a first polypeptide comprising the amino acid sequence of SEQ ID NO: 168, a second polypeptide comprising the amino acid sequence of SEQ ID NO: 171, and a third polypeptide comprising the amino acid sequence of SEQ ID NO: 148; or 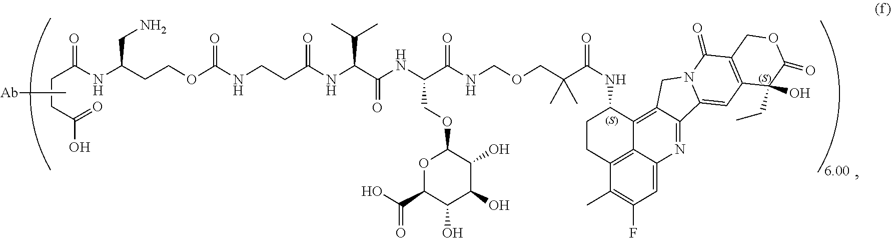
or a pharmaceutically acceptable salt, solvate, and/or stereoisomer thereof, wherein the Ab comprises a first polypeptide comprising the amino acid sequence of SEQ ID NO: 168, a second polypeptide comprising the amino acid sequence of SEQ ID NO: 171, and a third polypeptide comprising the amino acid sequence of SEQ ID NO: 148.

108. A pharmaceutical composition comprising the antibody drug conjugate of claim 82 and a pharmaceutically acceptable carrier.

109. A method of treating a cancer comprising administering to a patient in need a therapeutically effective amount of the pharmaceutical composition of claim 108.

110. The method of claim 109, wherein the cancer is non-small cell lung cancer (NSCLC) or small cell lung cancer (SCLC).

111. A process for producing the antibody drug conjugate of claim 82 comprising:
(i) culturing a host cell that has been transformed by an isolated nucleic acid comprising one or more sequences encoding the multispecific antibody or antigen-binding fragment thereof;
(ii) expressing the multispecific antibody or antigen-binding fragment thereof;
(iii) recovering the expressed multispecific antibody or antigen-binding fragment thereof; and
(iv) conjugating the payload (P) to the multispecific antibody or fragment thereof using the linker (L) and conjugator (C), such that the antibody drug conjugate is formed.
